SEQUENCE LISTING (162 sequences)

SEQ ID NO: 1 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
  LIPID 14: note = Lipidated Lysine
XNTXXXXXXENXXK

SEQ ID NO: 2 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 3 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
  LIPID 14: note = Lipidated Lysine
XNTXXXXXXENXXK

SEQ ID NO: 4 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  LIPID 11: note = Lipidated Lysine
  MOD_RES 13: note = 3-pyridinylalanine
  MOD_RES 14: note = MeGly
  LIPID 15: note = Lipidated Lysine
RXNTXXXXXXKNXXK

SEQ ID NO: 5 - protein, 14 aa, synthetic construct
  MOD_RES 2: note = Abu
  MOD_RES 6: note = N-acetyllysine
  MOD_RES 7: note = Alpha-methylcysteine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTWXXXXXENXX

SEQ ID NO: 6 - protein, 14 aa, synthetic construct
  MOD_RES 2: note = Abu
  MOD_RES 5: note = 7-methyltryptophan
  MOD_RES 6: note = N-acetyllysine
  MOD_RES 7: note = Alpha-methylcysteine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 7-8 - skipped (2 entries)

SEQ ID NO: 9 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid-N-LIPID
  MOD_RES 9: note = 2-amino-3-(naphthalen-2-yl)propanoic acid
  MOD_RES 10: note = 4-aminotetrahydro-2H-pyran-4-carboxylic acid
  MOD_RES 11: note = (2R)-2-(4-((5-amino-5-carboxypentyl)amino)-4-oxobut anamido)-3-carboxy-N,N,N-trimethylpropan-1-aminium
  MOD_RES 13: note = 3-pyridinylalanine
  MOD_RES 14: note = MeGly
RXNTXXXXXXXNXX

SEQ ID NO: 10 - protein, 14 aa, synthetic construct
  MOD_RES 2: note = Penicillamine
  MOD_RES 3: note = (2R)-2-(4-((5-amino-5-carboxypentyl)amino)-4-oxobut anamido)-3-carboxy-N,N,N-trimethylpropan-1-aminium
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid-N-LIPID
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXXTXXXXXXENXX

SEQ ID NO: 11 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid-N-LIPID
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 12 - protein, 14 aa, synthetic construct
  MOD_RES 2: note = Penicillamine
  LIPID 3: note = Lipidated Lysine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 11: note = (2R)-2-(4-((5-amino-5-carboxypentyl)amino)-4-oxobut anamido)-3-carboxy-N,N,N-trimethylpropan-1-aminium
  MOD_RES 13: note = 3-pyridinylalanine
  MOD_RES 14: note = MeGly
RXKTXXXXXXXNXX

SEQ ID NO: 13 - protein, 14 aa, synthetic construct
  MOD_RES 2: note = Penicillamine
  MOD_RES 5: note = 7-methyltryptophan
  MOD_RES 6: note = (2R)-2-(4-((5-amino-5-carboxypentyl)amino)-4-oxobut anamido)-3-carboxy-N,N,N-trimethylpropan-1-aminium
  MOD_RES 7: note = Penicillamine
  MOD_RES 8: note = 2-amino-3-(4-(2-aminoethoxy)phenyl)propanoic acid-N-LIPID
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 14 - skipped

SEQ ID NO: 15 - protein, 14 aa, synthetic construct
  MOD_RES 2: note = Penicillamine
  MOD_RES 5: note = 7-methyltryptophan
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 11: note = (2R)-2-(4-((5-amino-5-carboxypentyl)amino)-4-oxobut anamido)-3-carboxy-N,N,N-trimethylpropan-1-aminium
  MOD_RES 13: note = 3-pyridinylalanine
  MOD_RES 14: note = MeGly
RXNTXKXXXXXNXX

SEQ ID NO: 16 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = (2R)-2-(4-((2-(4-(2-amino-2-carboxyethyl)phenoxy)et hyl)amino)-4-oxobutanamido)-3-carboxy-N,N,N-trimethylpropa n-1-aminium
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = N-Methyl-Lysine(LIPID)
  LIPID 14: note = Lipidated N-Methyl-Lysine
RXNTXXXXXXENXX

SEQ ID NO: 17 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = (2R)-2-(4-((2-(4-(2-amino-2-carboxyethyl)phenoxy)et hyl)amino)-4-oxobutanamido)-3-carboxy-N,N,N-trimethylpropa n-1-aminium
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
KXNTXXXXXXENXX

SEQ ID NO: 18 - protein, 14 aa, synthetic construct
  MOD_RES 2: note = Penicillamine
  LIPID 3: note = Lipidated Lysine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXKTXXXXXXENXX

SEQ ID NO: 19 - protein, 14 aa, synthetic construct
  MOD_RES 2: note = Penicillamine
  MOD_RES 5: note = 7-methyltryptophan
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = (2R)-3-carboxy-2-(4-((5-carboxy-5-(methylamino)pent yl)amino)-4-oxobutanamido)-N,N,N-trimethylpropan-1-aminium
RXNTXKXXXXENXX

SEQ ID NO: 20 - protein, 14 aa, synthetic construct
  MOD_RES 2: note = Penicillamine
  MOD_RES 5: note = 7-methyltryptophan
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXKXXXXENXX

SEQ ID NO: 21 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  LIPID 11: note = Lipidated Lysine
  MOD_RES 13: note = 3-pyridinylalanine
  MOD_RES 14: note = (2R)-3-carboxy-2-(4-((5-carboxy-5-(methylamino)pent yl)amino)-4-oxobutanamido)-N,N,N-trimethylpropan-1-aminium
RXNTXXXXXXKNXX

SEQ ID NO: 22 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = (2R)-2-(4-((2-(4-(2-amino-2-carboxyethyl)phenoxy)et hyl)amino)-4-oxobutanamido)-3-carboxy-N,N,N-trimethylpropa n-1-aminium
  MOD_RES 9: note = 2-amino-3-(naphthalen-2-yl)propanoic acid
  MOD_RES 10: note = 4-aminotetrahydro-2H-pyran-4-carboxylic acid
  LIPID 11: note = Lipidated Lysine
  MOD_RES 13: note = 3-pyridinylalanine
  MOD_RES 14: note = MeGly
RXNTXXXXXXKNXX

SEQ ID NO: 23 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = (2R)-2-(4-((2-(4-(2-amino-2-carboxyethyl)phenoxy)et hyl)amino)-4-oxobutanamido)-3-carboxy-N,N,N-trimethylpropa n-1-aminium
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 24 - skipped

SEQ ID NO: 25 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 26 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
KXNTXXXXXXENXX

SEQ ID NO: 27 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
  LIPID 14: note = Lipidated Lysine
XNTXXXXXXENXXK

SEQ ID NO: 28 - skipped

SEQ ID NO: 29 - protein, 14 aa, synthetic construct
  MOD_RES 2: note = Penicillamine
  MOD_RES 5: note = 7-methyltryptophan
  MOD_RES 6: note = (2R)-2-(4-((5-amino-5-carboxypentyl)amino)-4-oxobut anamido)-3-carboxy-N,N,N-trimethylpropan-1-aminium
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  LIPID 14: note = Lipidated N-Methyl-Lysine
  MOD_RES 14: note = N-Methyl-Lysine(LIPID)
RXNTXXXXXXENXX

SEQ ID NO: 30 - protein, 14 aa, synthetic construct
  MOD_RES 2: note = Penicillamine
  MOD_RES 3: note = (2R)-2-(4-((5-amino-5-carboxypentyl)amino)-4-oxobut anamido)-3-carboxy-N,N,N-trimethylpropan-1-aminium
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = N-Methyl-Lysine(LIPID)
  LIPID 14: note = Lipidated N-Methyl-Lysine
RXXTXXXXXXENXX

SEQ ID NO: 31 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = N-Methyl-Lysine(LIPID)
  LIPID 14: note = Lipidated N-Methyl-Lysine
RXNTXXXXXXENXX

SEQ ID NO: 32 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = (2R)-2-(4-((2-(4-(2-amino-2-carboxyethyl)phenoxy)et hyl)amino)-4-oxobutanamido)-3-carboxy-N,N,N-trimethylpropa n-1-aminium
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 33 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 11: note = (2R)-2-(4-((5-amino-5-carboxypentyl)amino)-4-oxobut anamido)-3-carboxy-N,N,N-trimethylpropan-1-aminium
  MOD_RES 13: note = 3-pyridinylalanine
  MOD_RES 14: note = MeGly
KXNTXXXXXXXNXX

SEQ ID NO: 34 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = (2R)-2-(4-((5-amino-5-carboxypentyl)amino)-4-oxobut anamido)-3-carboxy-N,N,N-trimethylpropan-1-aminium
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
KXNTXXXXXXENXX

SEQ ID NO: 35 - protein, 14 aa, synthetic construct
  LIPID 1: note = Lipidated Lysine
  MOD_RES 2: note = Penicillamine
  MOD_RES 3: note = (2R)-2-(4-((5-amino-5-carboxypentyl)amino)-4-oxobut anamido)-3-carboxy-N,N,N-trimethylpropan-1-aminium
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
KXXTXXXXXXENXX

SEQ ID NO: 36 - protein, 14 aa, synthetic construct
  MOD_RES 2: note = Penicillamine
  MOD_RES 5: note = 7-methyltryptophan
  LIPID 6: note = Lipidated Lysine
  MOD_RES 7: note = Penicillamine
  MOD_RES 8: note = (2R)-2-(4-((2-(4-(2-amino-2-carboxyethyl)phenoxy)et hyl)amino)-4-oxobutanamido)-3-carboxy-N,N,N-trimethylpropa n-1-aminium
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXKXXXXENXX

SEQ ID NO: 37 - protein, 14 aa, synthetic construct
  MOD_RES 2: note = Penicillamine
  MOD_RES 3: note = (2R)-2-(4-((5-amino-5-carboxypentyl)amino)-4-oxobut anamido)-3-carboxy-N,N,N-trimethylpropan-1-aminium
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  LIPID 11: note = Lipidated Lysine
  MOD_RES 13: note = 3-pyridinylalanine
  MOD_RES 14: note = MeGly
RXXTXXXXXXKNXX

SEQ ID NO: 38 - protein, 15 aa, synthetic construct
  MOD_RES 2: note = Penicillamine
  MOD_RES 5: note = 7-methyltryptophan
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXQXXXXENXXK

SEQ ID NO: 39 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
KXNTXXXXXXXNXX

SEQ ID NO: 40 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 41 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 11: note = (2R)-2-(4-((5-amino-5-carboxypentyl)amino)-4-oxobut anamido)-3-carboxy-N,N,N-trimethylpropan-1-aminium
  MOD_RES 13: note = 3-pyridinylalanine
  MOD_RES 14: note = N-Methyl-Lysine(LIPID)
  LIPID 14: note = Lipidated N-Methyl-Lysine
RXNTXXXXXXXNXX

SEQ ID NO: 42 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = (2R)-3-carboxy-2-(4-((5-carboxy-5-(methylamino)pent yl)amino)-4-oxobutanamido)-N,N,N-trimethylpropan-1-aminium
KXNTXXXXXXENXX

SEQ ID NO: 43 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = (2R)-3-carboxy-2-(4-((5-carboxy-5-(methylamino)pent yl)amino)-4-oxobutanamido)-N,N,N-trimethylpropan-1-aminium
RXNTXXXXXXENXX

SEQ ID NO: 44 - skipped

SEQ ID NO: 45 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 11: note = (2R)-2-(4-((5-amino-5-carboxypentyl)amino)-4-oxobut anamido)-3-carboxy-N,N,N-trimethylpropan-1-aminium
  MOD_RES 13: note = 3-pyridinylalanine
  MOD_RES 14: note = MeGly
RXNTXXXXXXXNXX

SEQ ID NO: 46 - protein, 14 aa, synthetic construct
  MOD_RES 2: note = Penicillamine
  MOD_RES 5: note = 7-methyltryptophan
  MOD_RES 6: note = (2R)-2-(4-((5-amino-5-carboxypentyl)amino)-4-oxobut anamido)-3-carboxy-N,N,N-trimethylpropan-1-aminium
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 47 - protein, 14 aa, synthetic construct
  MOD_RES 2: note = Penicillamine
  MOD_RES 3: note = (2R)-2-(4-((5-amino-5-carboxypentyl)amino)-4-oxobut anamido)-3-carboxy-N,N,N-trimethylpropan-1-aminium
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXXTXXXXXXENXX

SEQ ID NO: 48 - protein, 14 aa, synthetic construct
  MOD_RES 2: note = Penicillamine
  MOD_RES 5: note = 7-methyltryptophan
  MOD_RES 6: note = (2R)-2-(4-((5-amino-5-carboxypentyl)amino)-4-oxobut anamido)-3-carboxy-N,N,N-trimethylpropan-1-aminium
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  LIPID 11: note = Lipidated Lysine
  MOD_RES 13: note = 3-pyridinylalanine
  MOD_RES 14: note = MeGly
RXNTXXXXXXKNXX

SEQ ID NO: 49 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  LIPID 11: note = Lipidated Lysine
  MOD_RES 13: note = 3-pyridinylalanine
  MOD_RES 14: note = MeGly
RXNTXXXXXXKNXX

SEQ ID NO: 50 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  MOD_RES 10: note = (2R)-2-(4-((5-amino-5-carboxypentyl)amino)-4-oxobut anamido)-3-carboxy-N,N,N-trimethylpropan-1-aminium
  MOD_RES 12: note = 3-pyridinylalanine
  MOD_RES 13: note = MeGly
  LIPID 14: note = Lipidated Lysine
XNTXXXXXXXNXXK

SEQ ID NO: 51 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 11: note = (2R)-2-(4-((5-amino-5-carboxypentyl)amino)-4-oxobut anamido)-3-carboxy-N,N,N-trimethylpropan-1-aminium
  MOD_RES 13: note = 3-pyridinylalanine
  MOD_RES 14: note = MeGly
  LIPID 15: note = Lipidated Lysine
RXNTXXXXXXXNXXK

SEQ ID NO: 52 - protein, 15 aa, synthetic construct
  MOD_RES 2: note = Penicillamine
  MOD_RES 5: note = 7-methyltryptophan
  MOD_RES 6: note = (2R)-2-(4-((5-amino-5-carboxypentyl)amino)-4-oxobut anamido)-3-carboxy-N,N,N-trimethylpropan-1-aminium
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 53 - protein, 15 aa, synthetic construct
  MOD_RES 2: note = Penicillamine
  MOD_RES 3: note = (2R)-2-(4-((5-amino-5-carboxypentyl)amino)-4-oxobut anamido)-3-carboxy-N,N,N-trimethylpropan-1-aminium
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXXTXXXXXXENXXK

SEQ ID NO: 54 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 55 - protein, 14 aa, synthetic construct
  MOD_RES 2: note = Penicillamine
  LIPID 3: note = Lipidated Lysine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXKTXXXXXXENXX

SEQ ID NO: 56 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 11: note = N-biotinylated lysine
  MOD_RES 13: note = 3-pyridinylalanine
  MOD_RES 14: note = MeGly
  LIPID 15: note = Lipidated Lysine
RXNTXXXXXXXNXXK

SEQ ID NO: 57 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 58 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
KXNTXXXXXXENXX

SEQ ID NO: 59 - protein, 14 aa, synthetic construct
  MOD_RES 2: note = Penicillamine
  MOD_RES 5: note = 7-methyltryptophan
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXKXXXXENXX

SEQ ID NO: 60 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 61 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 62 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 5-(4-(2-amino-2-carboxyethyl)phenoxy)-N,N,N-trimeth ylpentan-1-aminium
  MOD_RES 9: note = 2-amino-3-(naphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
KXNTXXXXXXXNXX

SEQ ID NO: 63 - skipped

SEQ ID NO: 64 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXXXXXXKNXX

SEQ ID NO: 65 - protein, 14 aa, synthetic construct
  MOD_RES 2: note = Penicillamine
  MOD_RES 5: note = 7-methyltryptophan
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXKXXXXENXX

SEQ ID NO: 66 - protein, 14 aa, synthetic construct
  MOD_RES 2: note = Penicillamine
  LIPID 3: note = Lipidated Lysine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXKTXXXXXXENXX

SEQ ID NO: 67 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 68 - skipped

SEQ ID NO: 69 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 5-(4-(2-amino-2-carboxyethyl)phenoxy)-N,N,N-trimeth ylpentan-1-aminium
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 70 - protein, 15 aa, synthetic construct
  MOD_RES 2: note = Penicillamine
  MOD_RES 5: note = 7-methyltryptophan
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXKXXXXENXXK

SEQ ID NO: 71 - protein, 14 aa, synthetic construct
  MOD_RES 2: note = Penicillamine
  MOD_RES 5: note = 7-methyltryptophan
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXKXXXXENXX

SEQ ID NO: 72 - protein, 14 aa, synthetic construct
  MOD_RES 2: note = Penicillamine
  MOD_RES 5: note = 7-methyltryptophan
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXKXXXXENXX

SEQ ID NO: 73 - protein, 14 aa, synthetic construct
  MOD_RES 2: note = Penicillamine
  LIPID 3: note = Lipidated Lysine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXKTXXXXXXENXX

SEQ ID NO: 74 - protein, 14 aa, synthetic construct
  MOD_RES 2: note = Penicillamine
  LIPID 3: note = Lipidated Lysine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  LIPID 11: note = Lipidated Lysine
  MOD_RES 13: note = 3-pyridinylalanine
  MOD_RES 14: note = MeGly
RXKTXXXXXXKNXX

SEQ ID NO: 75 - protein, 14 aa, synthetic construct
  MOD_RES 2: note = Penicillamine
  LIPID 3: note = Lipidated Lysine
  MOD_RES 5: note = 7-methyltryptophan
  MOD_RES 6: note = N-acetyllysine
  MOD_RES 7: note = Penicillamine
  MOD_RES 8: note = 5-(4-(2-amino-2-carboxyethyl)phenoxy)-N,N,N-trimeth ylpentan-1-aminium
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXKTXXXXXXENXX

SEQ ID NO: 76 - protein, 14 aa, synthetic construct
  MOD_RES 2: note = Penicillamine
  LIPID 3: note = Lipidated Lysine
  MOD_RES 5: note = 7-methyltryptophan
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXKTXKXXXXENXX

SEQ ID NO: 77 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXXXXXXKNXX

SEQ ID NO: 78 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXXXXXXKNXX

SEQ ID NO: 79 - protein, 14 aa, synthetic construct
  MOD_RES 2: note = Penicillamine
  MOD_RES 5: note = 7-methyltryptophan
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  LIPID 11: note = Lipidated Lysine
  MOD_RES 13: note = 3-pyridinylalanine
  MOD_RES 14: note = MeGly
RXNTXKXXXXKNXX

SEQ ID NO: 80 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = N-Methyl-Lysine(LIPID)
  LIPID 14: note = Lipidated N-Methyl-Lysine
RXNTXXXXXXENXX

SEQ ID NO: 81 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 82 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  LIPID 11: note = Lipidated Lysine
  MOD_RES 13: note = 3-pyridinylalanine
  MOD_RES 14: note = MeGly
RXNTXXXXXXKNXX

SEQ ID NO: 83 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 5-(4-(2-amino-2-carboxyethyl)phenoxy)-N,N,N-trimeth ylpentan-1-aminium
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXXXXXXENXX

SEQ ID NO: 84 - protein, 14 aa, synthetic construct
  MOD_RES 2: note = Penicillamine
  MOD_RES 5: note = 7-methyltryptophan
  LIPID 6: note = Lipidated Lysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXKXXXXENXX

SEQ ID NO: 85 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 5-(4-(2-amino-2-carboxyethyl)phenoxy)-N,N,N-trimeth ylpentan-1-aminium
  MOD_RES 8: note = 2-amino-3-(naphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXXXXXXKNXX

SEQ ID NO: 86 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXKXXXXKNXX

SEQ ID NO: 87-91 - skipped (5 entries)

SEQ ID NO: 92 - protein, 14 aa, synthetic construct
  MOD_RES 2: note = Penicillamine
  MOD_RES 5: note = 7-methyltryptophan
  LIPID 6: note = Lipidated Lysine
  MOD_RES 7: note = Penicillamine
  MOD_RES 8: note = 5-(4-(2-amino-2-carboxyethyl)phenoxy)-N,N,N-trimeth ylpentan-1-aminium
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXNTXKXXXXENXX

SEQ ID NO: 93 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 94-95 - skipped (2 entries)

SEQ ID NO: 96 - protein, 13 aa, synthetic construct
  MOD_RES 1: note = Penicillamine
  MOD_RES 4: note = 7-methyltryptophan
  LIPID 5: note = Lipidated Lysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXKXXXXENXX

SEQ ID NO: 97 - protein, 13 aa, synthetic construct
  MOD_RES 1: note = Penicillamine
  LIPID 2: note = Lipidated Lysine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XKTXXXXXXENXX

SEQ ID NO: 98 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
KXNTXXXXXXXNXX

SEQ ID NO: 99 - protein, 14 aa, synthetic construct
  MOD_RES 2: note = Penicillamine
  LIPID 3: note = Lipidated Lysine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXKTXXXXXXENXX

SEQ ID NO: 100 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXXXXXXKNXX

SEQ ID NO: 101 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 102 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  MOD_RES 10: note = N-acetyllysine
  MOD_RES 12: note = 3-pyridinylalanine
  MOD_RES 13: note = MeGly
  LIPID 14: note = Lipidated Lysine
XNTXXXXXXXNXXK

SEQ ID NO: 103 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
  LIPID 14: note = Lipidated Lysine
XNTXXXXXXENXXK

SEQ ID NO: 104 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 11: note = N-acetyllysine
  MOD_RES 13: note = 3-pyridinylalanine
  MOD_RES 14: note = MeGly
KXNTXXXXXXXNXX

SEQ ID NO: 105 - protein, 14 aa, synthetic construct
  MOD_RES 2: note = Penicillamine
  LIPID 3: note = Lipidated Lysine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
RXKTXXXXXXENXX

SEQ ID NO: 106-115 - skipped (10 entries)

SEQ ID NO: 116 - protein, 12 aa, synthetic construct
  MOD_RES 1: note = Penicillamine
  MOD_RES 4: note = N-acetyllysine
  MOD_RES 5: note = Penicillamine
  MOD_RES 6: note = 2-amino-3-(4-(2-aminoethoxy)phenyl)propanoic acid
  MOD_RES 7: note = 2-amino-3-(naphthalen-2-yl)propanoic acid
  MOD_RES 8: note = 4-aminotetrahydro-2H-pyran-4-carboxylic acid
  MOD_RES 11: note = 3-pyridinylalanine
  MOD_RES 12: note = MeGly
XNTXXXXXENXX

SEQ ID NO: 117 - protein, 14 aa, synthetic construct
  LIPID 1: note = Lipidated Lysine
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
KXNTXXXXXXENXX

SEQ ID NO: 118-132 - skipped (15 entries)

SEQ ID NO: 133 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 134 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 135 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 136-145 - skipped (10 entries)

SEQ ID NO: 146 - protein, 14 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  LIPID 11: note = Lipidated Lysine
  MOD_RES 13: note = 3-pyridinylalanine
  MOD_RES 14: note = MeGly
RXNTXXXXXXKNXX

SEQ ID NO: 147 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
XNTXXXXXXENXX

SEQ ID NO: 148 - skipped

SEQ ID NO: 149 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
  LIPID 14: note = Lipidated Lysine
XNTXXXXXXENXXK

SEQ ID NO: 150 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
  LIPID 14: note = Lipidated Lysine
XNTXXXXXXENXXK

SEQ ID NO: 151-159 - skipped (9 entries)

SEQ ID NO: 160 - protein, 15 aa, synthetic construct
  MOD_RES 2: note = Penicillamine
  MOD_RES 5: note = 7-methyltryptophan
  MOD_RES 6: note = N-acetyllysine
  MOD_RES 7: note = Penicillamine
  MOD_RES 8: note = 2-amino-3-(4-(2-aminoethoxy)phenyl)propanoic acid
  MOD_RES 9: note = 2-amino-3-(naphthalen-2-yl)propanoic acid
  MOD_RES 10: note = 4-aminotetrahydro-2H-pyran-4-carboxylic acid
  MOD_RES 13: note = 3-pyridinylalanine
  MOD_RES 14: note = MeGly
  LIPID 15: note = Lipidated Lysine
RXNTXXXXXXENXXK

SEQ ID NO: 161 - protein, 14 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  MOD_RES 12: note = 3-pyridinylalanine
  MOD_RES 13: note = MeGly
  LIPID 14: note = Lipidated Lysine
XNTXXXXXXENXXK

SEQ ID NO: 162 - protein, 13 aa, synthetic construct
  MOD_RES 1: note = Penicillamine
  MOD_RES 4: note = 7-methyltryptophan
  MOD_RES 5: note = N-acetyllysine
  MOD_RES 6: note = Penicillamine
  MOD_RES 7: note = 2-amino-3-(4-(2-aminoethoxy)phenyl)propanoic acid
  MOD_RES 8: note = 2-amino-3-(naphthalen-2-yl)propanoic acid
  MOD_RES 9: note = 4-aminotetrahydro-2H-pyran-4-carboxylic acid
  LIPID 10: note = Lipidated Lysine
  MOD_RES 12: note = 3-pyridinylalanine
  MOD_RES 13: note = MeGly
XNTXXXXXXKNXX

(19) United States
(12) Patent Application Publication
(10) Pub. No.: US 2026/0250320 A1
(43) Pub. Date: Aug. 27, 2026

(54) LIPIDATED PEPTIDE INHIBITORS OF INTERLEUKIN-23 RECEPTOR

(71) Applicant: Janssen Pharmaceutica NV, Beerse (BE)

(72) Inventors: Stephanie A. Barros, Chalfont, PA (US); Boying Guo, Warrington, PA (US); David Marcoux, Newton Centre, MA (US); Santhosh Francis Neelamkavil, Edison, NJ (US); Sandeep Somani, Princeton, NJ (US); Chengzao Sun, Warrington, PA (US); Tho Thieu, North Wales, PA (US); Jing Zhang, Wilmington, DE (US); Danila Branca, Pomezia (IT); Roberto Costante, Pomezia (IT)

(21) Appl. No.: 19/148,240

(22) PCT Filed: Jan. 15, 2024
(86) PCT No.: PCT/US2024/011550

§ 371 Date: Jul. 15, 2025

Related U.S. Application Data
(60) Provisional application No. 63/480,040, filed on Jan. 16, 2023.

(51) Int. Cl. C07K 7/56; A61K 38/00

(52) U.S. Cl. CPC C07K 7/56; A61K 38/00

(57) ABSTRACT

The present disclosure relates to peptide inhibitors of the interleukin-23 receptor (IL-23R) or pharmaceutically acceptable salts thereof, corresponding pharmaceutical compositions, methods and/or uses for treatment of autoimmune inflammation and related diseases and disorders.

### CROSS-REFERENCE TO RELATED APPLICATIONS

This application claims the benefit of U.S. Provisional Application No. 63/480,040 filed Jan. 16, 2023, which is herein incorporated by reference in its entirety.

### INCORPORATION OF SEQUENCE LISTING

The sequence listing in ST.26 XML format entitled 739654_NTT-4250PC_SL.xml, created on Jan. 15, 2024, comprising 491,164 bytes, prepared according to 37 CFR 1.822 to 1.824, submitted concurrently with the filing of this application, is incorporated herein by reference in its entirety.

### BACKGROUND

The interleukin-23 (IL-23) cytokine is a heterodimer composed of a unique p19 subunit and the p40 subunit shared with IL-12, which is a cytokine involved in the development of interferon-γ (IFN-γ)-producing T helper 1 (T_{H}1) cells. Although IL-23 and IL-12 both contain the p40 subunit, they have different phenotypic properties. For example, animals deficient in IL-12 are susceptible to inflammatory autoimmune diseases, whereas IL-23 deficient animals are resistant to these diseases, presumably due to a reduced number of CD4⁺ T cells producing IL-6, IL-17, and TNF in the CNS of IL-23-deficient animals. IL-23 binds to IL-23R, which is a heterodimeric receptor composed of IL-12Rβ1 and IL-23R subunits. Binding of IL-23 to IL-23R activates the Jak-Stat signaling molecules Jak2, Tyk2, Stat1, Stat 3, Stat 4, and Stat 5, although Stat4 activation is substantially weaker and different DNA-binding Stat complexes form in response to IL-23 as compared with IL-12. IL-23R associates constitutively with Jak2 and in a ligand-dependent manner with Stat3. In contrast to IL-12, which acts mainly on naive CD4(+) T cells, IL-23 preferentially acts on memory CD4(+) T cells.

IL-23 has been implicated as playing a crucial role in the pathogenesis of autoimmune inflammation and related diseases and disorders, such as multiple sclerosis, asthma, rheumatoid arthritis, psoriasis, and inflammatory bowel diseases (IBDs) such as ulcerative colitis and Crohn's disease. Studies in acute and chronic mouse models of IBDs revealed a primary role of interleukin-23 receptor (IL-23R) and downstream effector cytokines in disease pathogenesis. IL-23R is expressed on various adaptive and innate immune cells including Th17 cells, γδ T cells, natural killer (NK) cells, dendritic cells, macrophages, and innate lymphoid cells, which are found abundantly in the intestine. At the intestine mucosal surface, the gene expression and protein levels of IL-23R are found to be elevated in IBD patients. It is believed that IL-23 mediates this effect by promoting the development of a pathogenic CD4⁺ T cell population that produces IL-6, IL-17, and tumor necrosis factor (TNF).

Accordingly, there remains a need for compositions that bind IL-23R to inhibit IL-23 binding and signaling in a patient.

### BRIEF SUMMARY

Provided herein are peptide inhibitors of the interleukin-23 receptor (IL-23R) or pharmaceutically acceptable salts thereof, corresponding pharmaceutical compositions, and methods and/or uses of the IL-23R inhibitors for the treatment of inflammatory diseases, autoimmune diseases, and/or related disorders.

In particular, the present disclosure provides a peptide of Formula (I), comprising the amino acid sequence:

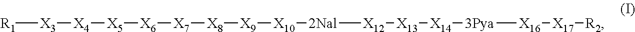

or a pharmaceutically acceptable salt thereof.

The present disclosure further provides a pharmaceutical composition comprising a peptide described herein, or a pharmaceutically acceptable salt thereof, and a pharmaceutically acceptable carrier.

The present disclosure still further provides a method for treating a disease or disorder associated with Interleukin 23 (IL-23)/Interleukin 23 Receptor (IL-23R), comprising administering to a subject in need thereof a therapeutically effective amount of a peptide or a pharmaceutical composition described herein. In some embodiments, the disease or disorder is selected from ulcerative colitis (UC), Crohn's disease (CD), psoriasis (PsO), and psoriatic arthritis (PsA).

### DETAILED DESCRIPTION

Provided herein are peptide inhibitors of IL-23R, and pharmaceutically acceptable salts thereof, corresponding pharmaceutical compositions, and methods and/or uses for the treatment of inflammatory diseases, autoimmune diseases, and/or related disorders.

### Definitions

Unless otherwise defined herein, scientific and technical terms used in this application shall have the meanings that are commonly understood by those of ordinary skill in the art.

As used in the specification and in the claims, the “comprise(s),” “comprising,” “include(s),” “having,” “has,” “can,” “contain(s),” and variants thereof, as used herein, are intended to be open-ended transitional phrases, terms, or words that require the presence of the named features, groups, ingredients, or steps and does not exclude the presence of additional features, groups, ingredients, or steps. For example, the language “a peptide of Formula (I), comprising the amino acid sequence: X₃—X₄—X₅—X₆—X₇—X₈—X₉—X₁₀—X₁₁-2Nal-X₁₃—X₁₄-3Pya-X₁₆—X₁₇ (I),” means that in addition to amino acids X₃ through X₁₇, the peptide may include but is not limited to additional amino acids attached to the N-terminus, additional amino acids attached to the C-terminus, N-terminal or C-terminal capping groups, chemical or biological moieties (including but not limited to, for example, lipophilic substituents, antibodies, imaging agents, etc.) conjugated to the peptide at any location, and the like. The term “comprise(s),” “comprising,” “include(s),” “having,” “has,” “can,” or “contain(s),” can include embodiments encompassed by the term “consisting essentially of” or “consisting of.”

The terms “peptide,” “polypeptide,” and “protein” are used interchangeably herein and typically refer to a molecule comprising a chain of two or more amino acids (e.g., L-amino acids, D-amino acids, modified amino acids, amino acid analogs, amino acid mimetics, etc.).

Unless otherwise indicated, naturally-occurring L-amino acids and D-amino acids are both represented by either conventional three-letter, or capitalized one-letter, amino acid designations of Table 1. In some embodiments, naturally-occurring L-amino acids are represented by either conventional three-letter, or capitalized one-letter, amino acid designations of Table 1. In some embodiments, D-amino acids, are represented by lower-case one-letter amino acid designations corresponding to one-letter designations of Table 1, i.e., g, a, 1, m, f, w, k, q, e, s, p, v, i, c, y, h, r, n, d, and t.

TABLE 1
Naturally-occurring amino acids

| | | | | | |
|---|---|---|---|---|---|
| G | Glycine | Gly | P | Proline | Pro |
| A | Alanine | Ala | V | Valine | Val |
| L | Leucine | Leu | I | Isoleucine | Ile |
| M | Methionine | Met | C | Cysteine | Cys |
| F | Phenylalanine | Phe | Y | Tyrosine | Tyr |
| W | Tryptophan | Trp | H | Histidine | His |
| K | Lysine | Lys | R | Arginine | Arg |
| Q | Glutamine | Gln | N | Asparagine | Asn |
| E | Glutamic Acid | Glu | D | Aspartic Acid | Asp |
| S | Serine | Ser | T | Threonine | Thr |

The term “L-amino acid,” as used herein, refers to the “L” isomeric form of an amino acid, and conversely the term “D-amino acid” refers to the “D” isomeric form of an amino acid (e.g., (D)Asp or D-Asp; (D)Phe or D-Phe). Amino acid residues in the D isomeric form can be substituted for any L-amino acid residue, as long as the desired function is retained by the peptide. D-amino acids may be indicated as customary in lower case when referred to using single-letter abbreviations. For example, D-arginine can be represented as “arg” or “r.” Alternatively, a lower case “d” in front of an amino acid can be used to indicate that it is of the D isomeric form, for example D-lysine can be represented by dK.

In the case of less common or non-naturally occurring amino acids, unless they are referred to by their full name (e.g., sarcosine, ornithine, etc.), frequently employed three- or four-character codes are employed for residues thereof, including, Sar or Sarc (sarcosine, i.e., N-methylglycine), Aib (α-aminoisobutyric acid), Dab (2,4-diaminobutanoic acid), Dapa (2,3-diaminopropanoic acid), γ-Glu (γ-glutamic acid), Gaba (γ-aminobutanoic acid), β-Pro (pyrrolidine-3-carboxylic acid), and Abu (2-aminobutyric acid).

Amino acids of the D-isomeric form may be located at any of the positions in the IL-23R inhibitors set forth herein (e.g., any of X₃—X₁₇ appearing in the molecule). In some embodiments, amino acids of the D-isomeric form may be located only at any one or more of X₃, X₅, X₆, X₈, X₁₃, and optionally one additional position. In other embodiments, amino acids of the D-isomeric form may be located only at any one or more of X₃, X₈, X₁₃, and optionally one additional position. In other embodiments, amino acids of the D-isomeric form may be located only at any one or more of X₈, X₁₃, and optionally one additional position. In other embodiments, amino acids of the D-isomeric form may be located only at X₃ and optionally one additional position. In other embodiments, amino acids of the D-isomeric form may be located only at X₃, and optionally two or three additional positions. In other embodiments, amino acids of the D-isomeric form may be located at only one or two of positions X₃ to X₁₇ appearing in the IL-23R inhibitors set forth herein. In other embodiments, amino acids of the D-isomeric form may be located at only three or four of positions X₃ to X₁₇ appearing in the IL-23R inhibitors set forth herein. For example, an IL-23R inhibitor set forth herein having only positions X₃ to X₁₅ present may have amino acids of the D-form present in three or four of those positions. In other embodiments, amino acids of the D-isomeric form may be located at only five or six of positions X₃ to X₁₇ appearing in the IL-23R inhibitors set forth herein.

Peptides may be naturally occurring, synthetically produced, or recombinantly expressed. Peptides may also comprise additional groups modifying the amino acid chain, for example, functional groups added via post-translational modification. Examples of post-translation modifications include, but are not limited to, acetylation, alkylation (including, methylation), biotinylation, glutamylation, glycylation, glycosylation, isoprenylation, lipoylation, phosphopantetheinylation, phosphorylation, selenation, and C-terminal amidation. The term peptide also includes peptides comprising modifications of the amino terminus and/or the carboxy terminus. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C1-C4 alkyl). The term peptide also includes modifications, such as but not limited to those described above, of amino acids falling between the amino and carboxy termini.

As is clear to the skilled artisan, the peptide sequences disclosed herein are shown proceeding from left to right, with the left end of the sequence being the N-terminus of the peptide and the right end of the sequence being the C-terminus of the peptide. Among sequences disclosed herein are sequences incorporating either an “—OH” moiety or an “—NH₂” moiety at the carboxy terminus (C-terminus) of the sequence. In such cases, and unless otherwise indicated, an “—OH” or an “—NH₂” moiety at the C-terminus of the sequence indicates a hydroxy group or an amino group, corresponding to the presence of a carboxylic acid (COOH) or an amido (CONH₂) group at the C-terminus, respectively. In each sequence of the disclosure, a C-terminal “—OH” moiety may be substituted for a C-terminal “—NH₂” moiety, and vice-versa.

The phrase “amino acid,” “amino acid residue,” or “residue” as used herein refers to an amino acid, a modified amino acid, an amino acid analog, or an amino acid mimetic that is incorporated into a peptide by an amide bond or an amide bond mimetic.

Unless indicated otherwise the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader. In sequences of amino acids that represent IL-23 inhibitors the individual amino acids are separated by a hyphen “-” or brackets e.g, lysine is shown as [K].

One of skill in the art will appreciate that certain amino acids and other chemical moieties are modified when bound to another molecule. For example, an amino acid side chain may be modified when it forms an intramolecular bridge with another amino acid side chain, e.g., one or more hydrogens may be removed or replaced by the bond.

The term “therapeutically effective amount” or “pharmaceutically effective amount” means that amount of active peptide or pharmaceutical agent that elicits the biological or medicinal response in a tissue system, animal or human, that is being sought by a researcher, veterinarian, medical doctor, or other clinician, which includes preventing, treating or ameliorating the symptoms of a syndrome, disorder or disease being treated.

The term “pharmaceutically acceptable” means approved or approvable by a regulatory agency of Federal or a state government or the corresponding agency in countries other than the United States, or that is listed in the U. S. Pharmacopoeia or other generally recognized pharmacopoeia for use in animals, and more particularly, in humans.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Composition” or “pharmaceutical composition” as used herein is intended to encompass a product comprising the specified active pharmaceutical ingredient (API) (i.e., a peptide of the present disclosure), which may include pharmaceutically acceptable excipients, carriers or diluents as described herein, such as in specified amounts defined throughout the disclosure.

Compositions or pharmaceutical compositions of the present disclosure may be in different pharmaceutically acceptable forms, which may include, but are not limited to a liquid composition, a tablet or matrix composition, a capsule composition, etc. When the composition is a tablet composition, the tablet may include, but is not limited to different layers two or more different phases, including an internal phase and an external phase that can comprise a core. The tablet composition can also include, but is not limited to one or more coatings.

Provided are also pharmaceutically acceptable salts and tautomeric forms of the peptides described herein.

A “pharmaceutically acceptable salt” is intended to mean a salt of a free acid or base of peptides represented by Formula (I) that are non-toxic, biologically tolerable, or otherwise biologically suitable for administration to the subject. It should possess the desired pharmacological activity of the parent compound. See, generally, G. S. Paulekuhn, et al., “Trends in Active Pharmaceutical Ingredient Salt Selection based on Analysis of the Orange Book Database”, J. Med. Chem., 2007, 50:6665-72, S. M. Berge, et al., “Pharmaceutical Salts”, J Pharm Sci., 1977, 66:1-19, and Handbook of Pharmaceutical Salts, Properties, Selection, and Use, Stahl and Wermuth, Eds., Wiley-VCH and VHCA, Zurich, 2002. Examples of pharmaceutically acceptable salts are those that are pharmacologically effective and suitable for contact with the tissues of patients without undue toxicity, irritation, or allergic response. A peptide of Formula (I) may possess a sufficiently acidic group, a sufficiently basic group, or both types of functional groups, and accordingly react with a number of inorganic or organic bases, and inorganic and organic acids, to form a pharmaceutically acceptable salt.

The IL-23R inhibitors of the present disclosure, pharmaceutically acceptable salts, and/or other forms thereof may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms of the IL-23R inhibitors of the present disclosure. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the aspect encompasses, but is not limited to, the specific diastereomerically or enantiomerically enriched form. Where chirality is not specified but is present, it is understood that the aspect is directed to either the specific diastereomerically or enantiomerically enriched form; or a racemic or scalemic mixture of such compound(s).

“Racemates” refers to a mixture of enantiomers. The mixture can include equal or unequal amounts of each enantiomer.

“Stereoisomer” and “stereoisomers” refer to compounds that differ in the chirality of one or more stereo centers. Stereoisomers include enantiomers and diastereomers. The compounds may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of Advanced Organic Chemistry, 4th ed., J. March, John Wiley and Sons, New York, 1992).

“Diastereoisomers” are stereoisomers that have at least two asymmetric atoms, but which are not mirror images of each other.

“Enantiomers” are a pair of stereoisomers that are non-superimposable mirror images of each other. A “racemic” mixture is a 1:1 mixture of a pair of enantiomers. A “scalemic” mixture of enantiomers is mixture of enantiomers at a ratio other than 1:1.

“Tautomer” refers to alternate forms of a compound that differ in the position of a proton, such as enol-keto and imine-enamine tautomers, or the tautomeric forms of heteroaryl groups containing a ring atom attached to both a ring —NH— and a ring ═N— such as pyrazoles, imidazoles, benzimidazoles, triazoles, and tetrazoles.

The term “administering” with respect to the methods of the present disclosure, means a method for therapeutically or prophylactically preventing, treating or ameliorating a syndrome, disorder or disease as described herein by using a compound of the disclosure, or pharmaceutically acceptable salt thereof, composition thereof, or medicament thereof. Such methods include administering a therapeutically effective amount of a peptide of the disclosure, or pharmaceutically acceptable salt thereof, composition thereof, or medicament thereof, at different times during the course of a therapy or concurrently or sequentially as a combination therapy.

“Patient” or “subject”, which are used interchangeably, refer to a living organism, preferably a mammal, most preferably a human, whom will be or has been treated by a method according to an embodiment of the application. Examples of mammals include, but are not limited to, cows, horses, sheep, pigs, cats, dogs, mice, rats, rabbits, guinea pigs, non-human primates (NHPs) such as monkeys or apes, humans, etc., more preferably a human.

As used herein, the term “treatment” or “treating,” is defined as the application or administration of a therapeutic agent, i.e., a compound of the present disclosure (alone or in combination with another pharmaceutical agent), to a patient, or application or administration of a therapeutic agent to an isolated tissue or cell line from a patient (e.g., for diagnosis or ex vivo applications), who has a disorder or disease as described herein, a symptom thereof, or the potential to develop such disorder or disease, where the purpose of the application or administration is to cure, heal, alleviate, relieve, alter, remedy, ameliorate, improve or affect the disorder or disease, its symptoms, or the potential to develop said disorder or disease. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

Unless defined otherwise, all technical and scientific terms used herein have the same meaning as commonly or conventionally understood by one of ordinary skill in the art. In the chemical arts a dash at the front or end of a chemical group is a matter of convenience; chemical groups may be depicted with or without one or more dashes without losing their ordinary meaning. A wavy line drawn through a line in a structure indicates a point of attachment of a group. A dashed line indicates an optional bond. Unless chemically or structurally required, no directionality is indicated or implied by the order in which a chemical group is written or the point at which it is attached to the remainder of the molecule. For instance, the group “—SO₂CH₂-” is equivalent to “—CH₂SO₂—” and both may be connected in either direction. Similarly, an “arylalkyl” group, for example, may be attached to the remainder of the molecule at either an aryl or an alkyl portion of the group. A prefix such as “Cᵤ₋ᵥ” or (Cᵤ-Cᵥ) indicates that the following group has from u to v carbon atoms. For example, “C₁₋₆alkyl” and “C₁-C₆ alkyl” both indicate that the alkyl group has from 1 to 6 carbon atoms.

The term “alkyl” is a straight or branched saturated hydrocarbon. For example, an alkyl group can have 1 to 10 carbon atoms (i.e., (C₁-C₁₀)alkyl), 1 to 5 carbon atoms (i.e., (C₁-C₅)alkyl), 1 to 4 carbon atoms (i.e., (C₁-C₄)alkyl), or 1 to 3 carbon atoms (i.e., (C₁-C₃)alkyl). Examples of alkyl groups include, but are not limited to, methyl (Me, —CH₃), ethyl (Et, —CH₂CH₃), 1-propyl (n-Pr, n-propyl, —CH₂CH₂CH₃), isopropyl (i-Pr, i-propyl, —CH(CH₃)₂), 1-butyl (n-bu, n-butyl, —CH₂CH₂CH₂CH₃), 2-butyl (s-bu, s-butyl, —CH(CH₃)CH₂CH₃), tert-butyl (t-bu, t-butyl, —CH(CH₃)₃), 1-pentyl (n-pentyl, —CH₂CH₂CH₂CH₂CH₃), 2-pentyl (—CH(CH₃) CH₂CH₂CH₃), neopentyl (—CH₂C(CH₃)₃), 1-hexyl (—CH₂CH₂CH₂CH₂CH₂CH₃), 2-hexyl (—CH(CH₃)CH₂CH₂CH₂CH₃), heptyl (—(CH₂)₆CH₃), octyl (—(CH₂)₇CH₃), 2,2,4-trimethylpentyl (—CH₂C(CH₃)₂CH₂CH(CH₃)₂), nonyl (—(CH₂)₈CH₃), decyl (—(CH₂)₉CH₃), undecyl (—(CH₂)₁₀CH₃), and dodecyl (—(CH₂)₁₁CH₃). In an embodiment, alkyl refers to C₍₁₋₆₎alkyl. In another embodiment, alkyl refers to C₍₁₋₄₎alkyl. In another embodiment, alkyl refers to C₍₁₋₃₎alkyl.

Furthermore, it is intended that within the scope of the present invention, any element, in particular when mentioned in relation to a peptide of the disclosure, or pharmaceutically acceptable salt thereof, shall comprise all isotopes and isotopic mixtures of said element, either naturally occurring or synthetically produced, either with natural abundance or in an isotopically enriched form. For example, a reference to hydrogen includes within its scope ¹H, ²H (i.e., deuterium or D), and ³H (i.e., tritium or T). In some embodiments, the compounds described herein include a ²H (i.e., deuterium) isotope. By way of example, the group denoted —C₍₁₋₆₎alkyl includes not only —CH₃, but also CD₃; not only CH₂CH₃, but also CD₂CD₃, etc. Similarly, references to carbon and oxygen include within their scope respectively ¹²C, ¹³C and ¹⁴C and ¹⁵O and ¹⁶O and ¹⁷O and ¹⁸O. The isotopes may be radioactive or non-radioactive. Radiolabelled compounds of the disclosure may include a radioactive isotope selected from the group comprising ³H, ¹¹C, ¹⁸F, ³⁵S, ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ³H, ¹¹C and ¹⁸F.

Abbreviation, “(V/V)” refers to the phrase “volume for volume”, i.e., the proportion of a particular substance within a mixture, as measured by volume or a volume amount of a component of the composition disclosed herein relative to the total volume amount of the composition. Accordingly, the quantity is unit less and represents a volume percentage amount of a component relative to the total volume of the composition. For example, a 2% (V/V) solvent mixture can indicate 2 mL of one solvent is present in 100 mL of the solvent mixture.

“Bioavailability” refers to the extent and rate at which the active moiety (drug or metabolite) enters systemic circulation, thereby accessing the site of action. Bioavailability of a drug could be impacted by the factors such as properties of the dosage form and properties of the drug.

“Digestive tract tissue” as used herein refers to all the tissues that comprise the organs of the alimentary canal. For example only, and without limitation, “digestive tract tissue” includes tissues of the mouth, esophagus, stomach, small intestine, large intestine, duodenum, and anus.

### Compounds

The present disclosure provides a peptide inhibitor of interleukin-23 receptor. In particular, the present disclosure provides a peptide of Formula (I), comprising the amino acid sequence:

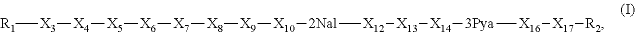

or a pharmaceutically acceptable salt thereof, wherein:
R₁ is MeCO, Z_{peg}, Z_{lipid}, succiniccarn, 5cpaCO, 8Aoc, 7Ahp, 6Ahx, 5Ava, or AEEP-Z_{lipid};
X₃ is R, K—Z_{lipid}, Dab-Z_{lipid}, NMeK—Z_{lipid}, a ring-forming amino acid, or absent;
X₄ is any amino acid;
X₅ is N, N(NMe₂), K(d), K—Z_{lipid}, Dab-Z_{lipid}, NMeK—Z_{lipid}, or a ring-forming amino acid;
X₆ is T, K—Z_{lipid}, NMeK—Z_{lipid}, Dab-Z_{lipid}, or a ring-forming amino acid;
X₇ is 7MeW, W, or absent;
X₈ is K(Ac), K(d), K(NMeAc), Q, K—Z_{lipid}, K—Z_{peg}, NMeK—Z_{lipid}, Dab-Z_{lipid}, Dab(NMecarn), Dab-Z_{peg}, or a ring-forming amino acid;
X₉ is any amino acid;
X₁₀ is AEF, TMAPF, AEF(d), TMAPF-Z_{lipid}, APEG3F, AEF-Z_{lipid}, or a ring-forming amino acid;
X₁₂ is THP, K—Z_{lipid}, Dab-Z_{lipid}, NMeK—Z_{lipid}, or a ring-forming amino acid;
X₁₃ is E, K(Ac), K(d), K—Z_{peg}, K(NMeAc), Dab(NMecarn), E(OAll), K—Z_{lipid}, Dab-Z_{lipid}, NMeK—Z_{lipid}, absent, or a ring-forming amino acid;
X₁₄ is N, K—Z_{lipid}, NMeK—Z_{lipid}, Dab-Z_{lipid}, or a ring-forming amino acid;
X₁₆ is Sar, NMeK(d), K—Z_{lipid}, Dab-Z_{lipid}, NMeK—Z_{lipid}, or a ring-forming amino acid;
X₁₇ is K—Z_{lipid}, Dab-Z_{lipid}, NMeK—Z_{lipid}, a ring-forming amino acid, or absent;
R₂ is CONH2, CONMe2, CONH—Z_{peg}, or CO—Z_{lipid};
Z_{peg}, independently for each occurrence, is a polyethylene glycol chain;
Z_{lipid}, independently for each occurrence, is a lipophilic substituent;
wherein the polypeptide comprises a first ring-forming amino acid that is linked to a second ring-forming amino acid to form a ring comprising 4-11 or 14 amino acids; or
wherein the polypeptide comprises a ring-forming amino acid that is linked to the C-terminus of the polypeptide to form a ring comprising 4-11 or 14 amino acids; and
wherein the polypeptide comprises at least one lipophilic substituent; provided that the polypeptide is not selected from the group consisting of:
MeCO-r-Pen-N-T-7MeW-K (Ac)-Pen-AEF-2Nal-THP-E-N-3Pya-Sar-K (PEG2PEG2gEC18OH)-CONH2,
MeCO-r-Pen-N-T-7MeW-K (Ac)-Pen-AEF-2Nal-THP-E-N-3Pya-NMeK (PEG2PEG2gEC18OH)-CONH2,
MeCO-Pen-N-T-7MeW-K (Ac)-Pen-AEF-2Nal-THP-K (PEG2PEG2gEC18OH)-N-3Pya-Sar-CONH2,
MeCO-k (PEG2PEG2gEC18OH)-Pen-N-T-7MeW-K (Ac)-Pen-AEF-2Nal-THP-K (Ac)-N -3Pya-Sar-CONH2,
MeCO-r-Pen-N-T-7MeW-K (Ac)-Pen-AEF (PEG2PEG2gEC18OH)-2Nal-THP-K (Ac)-N -3Pya-Sar-CONH2,
MeCO-k (PEG2PEG2SP6gEC18OH)-Pen-N-T-7MeW-K (Ac)-Pen-AEF-2Nal-THP-K (Ac)-N-3Pya-Sar-CONH2,
MeCO-k (PEG2PEG2gEmXOH)-Pen-N-T-7MeW-K (Ac)-Pen-AEF-2Nal-THP-K (PEG2PEG2gEmXOH)-N-3Pya-Sar-CONH2,
MeCO-k (PEG2PEG2gEC16OH)-Pen-N-T-7MeW-K (Ac)-Pen-AEF-2Nal-THP-K (gEC16)-N-3Pya-Sar-CONH2,
HOC18gEPEG2PEG2-r-Pen-N-T-7MeW-K (Ac)-Pen-AEF-2Nal-THP-K (Ac)-N-3Pya-Sar -CONH2,
HOC18gEPEG2PEG2-r-Pen-N-T-7MeW-K (Ac)-Pen-AEF-2Nal-THP-K (PEG2PEG2gEC18OH)-N-3Pya-Sar-CONH2,
MeCO-k (PEG2PEG2gEDab (mXOH) 2)-Pen-N-T-7MeW-K (Ac)-Pen-AEF-2Nal-THP-K (Ac)-N-3Pya-Sar-CONH2,
MeCO-k (PEG2PEG2gE (c) C18OH)-Pen-N-T-7MeW-K (Ac)-Pen-AEF-2Nal-THP-K (Ac)-N-3Pya-Sar-CONH2,
MeCO-r-Pen-N-T-7MeW-K (Ac)-Pen-AEF-2Nal-THP-E-N-3Pya-Sar-K (PEG2PEG2gEC16OH)-CONH2,
MeCO-r-Pen-N-T-7MeW-K (Ac)-Pen-AEF-2Nal-THP-E-N-3Pya-K (PEG2PEG2gEC18OH)-CONH2,
MeCO-Pen-N-T-7MeW-K (Ac)-Pen-AEF-2Nal-THP-K (PEG2PEG2gEC16OH)-N-3Pya-Sar -CONH2,
MeCO-Pen-K (PEG2PEG2gEC18OH)-T-7MeW-K (Ac)-Pen-AEF-2Nal-THP-E-N-3Pya-Sar -CONH2,
MeCO-r-Pen-K (PEG2PEG2gEC16OH)-T-7MeW-K (Ac)-Pen-AEF-2Nal-THP-E-N-3Pya-Sar-CONH2,
MeCO-r-Pen-N-T-7MeW-K (Ac)-Pen-AEF-2Nal-THP-E-N-3Pya-NMeK (PEG2PEG2gEC16OH)-CONH2,
MeCO-r-Pen-N-T-7MeW-K (Ac)-Pen-AEF-2Nal-THP-E-N-3Pya-K (PEG2PEG2gEC16OH)-CONH2,
MeCO-k (PEG2PEG2gEC16OH)-Pen-N-T-7MeW-K (Ac)-Pen-AEF-2Nal-THP-K (Ac)-N -3Pya-Sar-CONH2,
MeCO-r-Pen-N-T-7MeW-K (Ac)-Pen-AEF (PEG2PEG2gEC16OH)-2Nal-THP-K (Ac)-N -3Pya-Sar-CONH2,
MeCO-r-Pen-N-T-7MeW-K (Ac)-Pen-AEF-2Nal-THP-K (PEG2PEG2gEC18OH)-N-3Pya-Sar-CONH2,
MeCO-r-Pen-N-T-7MeW-K (Ac)-Pen-AEF-2Nal-THP-K (PEG2PEG2gEC16OH)-N-3Pya-Sar-CONH2,
MeCO-Pen-N-T-7MeW-K (Ac)-Pen-AEF-2Nal-THP-K (gEC16OH)-N-3Pya-Sar-CONH2,
MeCO-Pen-N-T-7MeW-K (Ac)-Pen-AEF-2Nal-THP-K (gEC18OH)-N-3Pya-Sar-CONH2,
MeCO-Pen-N-T-7MeW-K (Ac)-Pen-AEF-2Nal-THP-K (gEC18)-N-3Pya-Sar-CONH2,
MeCO-Pen-N-T-7MeW-K (Ac)-Pen-AEF-2Nal-THP-K (PEG6gEC16OH)-N-3Pya-Sar-CONH2,
MeCO-Pen-N-T-7MeW-K (Ac)-Pen-AEF-2Nal-THP-K (PEG6gEC18OH)-N-3Pya-Sar-CONH2,
MeCO-Pen-N-T-7MeW-K (Ac)-Pen-AEF-2Nal-THP-K (PEG2gEC16OH)-N-3Pya-Sar-CONH2,
MeCO-Pen-N-T-7MeW-K (Ac)-Pen-AEF-2Nal-THP-K (PEG2gEC18OH)-N-3Pya-Sar- CONH2,
MeCO-Pen-N-T-7MeW-K (Ac)-Pen-AEF-2Nal-THP-K (PEG2PEG2gEC20OH)-N-3Pya-Sar-CONH2,
HOC16gEPEG2PEG2-r-Pen-N-T-7MeW-K (Ac)-Pen-AEF-2Nal-THP-K (PEG2PEG2gEC16OH)-N-3Pya-Sar-CONH2,
MeCO-Pen-N-T-7MeW-K (Ac)-Pen-AEF-2Nal-THP-K (PEG2PEG6gEC16OH)-N-3Pya-Sar-CONH2,
MeCO-Pen-N-T-7MeW-K (Ac)-Pen-AEF-2Nal-THP-K (PEG2PEG6gEC18OH)-N-3Pya-Sar-CONH2,
MeCO-Pen-N-T-7MeW-K (Ac)-Pen-AEF-2Nal-THP-K (PEG2PEG2C16OH)-N-3Pya-Sar-CONH2,
MeCO-Pen-N-T-7MeW-K (Ac)-Pen-AEF-2Nal-THP-K (PEG2PEG2C18OH)-N-3Pya-Sar-CONH2,
MeCO-Pen-N-T-7MeW-K (Ac)-Pen-AEF-2Nal-THP-K (PEG6PEG6gEC16OH)-N-3Pya-Sar-CONH2,
MeCO-Pen-N-T-7MeW-K (Ac)-Pen-AEF-2Nal-THP-K (PEG6PEG6gEC18OH)-N-3Pya-Sar-CONH2,
MeCO-Pen-N-T-7MeW-K (Ac)-Pen-AEF-2Nal-THP-K (PEG24-gEC16OH)-N-3Pya-Sar-CONH2,
MeCO-Pen-N-T-7MeW-K (Ac)-Pen-AEF-2Nal-THP-K (PEG24-gEC18OH)-N-3Pya-Sar-CONH2,
MeCO-Pen-N-T-7MeW-K (Ac)-Pen-AEF-2Nal-THP-K (PEG24-C18OH)-N-3Pya-Sar-CONH2,
MeCO-k (gEC18OH)-Pen-N-T-7MeW-K (Ac)-Pen-AEF-2Nal-THP-K (Ac)-N-3Pya-Sar-CONH2,
MeCO-k (PEG2gEC18OH)-Pen-N-T-7MeW-K (Ac)-Pen-AEF-2Nal-THP-K (Ac)-N-3Pya-Sar-CONH2,
MeCO-r-Pen-K (PEG2PEG2gEC18OH)-T-7MeW-K (Ac)-Pen-AEF-2Nal-THP-K (Ac)-N -3Pya-Sar-CONH2,
MeCO-r-Pen-K (gEC18OH)-T-7MeW-K (Ac)-Pen-AEF-2Nal-THP-K (Ac)-N-3Pya-Sar-CONH2,
MeCO-r-Pen-K (PEG2gEC18OH)-T-7MeW-K (Ac)-Pen-AEF-2Nal-THP-K (Ac)-N-3Pya-Sar-CONH2,
MeCO-r-Pen-K (PEG2PEG2C18OH)-T-7MeW-K (Ac)-Pen-AEF-2Nal-THP-K (Ac)-N-3Pya-Sar-CONH2,
MeCO-r-Pen-N-T-7MeW-K (Ac)-Pen-AEF-2Nal-THP-K (Ac)-N-3Pya-NMeK (PEG2PEG2gEC18OH)-CONH2,
MeCO-r-Pen-N-T-7MeW-K (Ac)-Pen-AEF-2Nal-THP-K (PEG2PEG2-DgEC18OH)-N-3Pya-Sar-CONH2,
MeCO-r-Pen-N-T-7MeW-K (Ac)-Pen-AEF-2Nal-THP-K (PEG2PEG2-pC18OH)-N-3Pya-Sar-CONH2,
MeCO-r-Pen-N-T-7MeW-K (Ac)-Pen-AEF-2Nal-THP-K (PEG2PEG2gETrxC18OH)-N-3Pya-Sar-CONH2,
MeCO-r-Pen-N-T-7MeW-K (Ac)-Pen-AEF-2Nal-THP-K (PEG2PEG2gETrxC20OH)-N-3Pya-Sar-CONH2,
MeCO-k (PEG6gEC18OH)-Pen-N-T-7MeW-K (Ac)-Pen-AEF-2Nal-THP-K (Ac)-N-3Pya-Sar-CONH2,
MeCO-k (PEG2PEG6gEC18OH)-Pen-N-T-7MeW-K (Ac)-Pen-AEF-2Nal-THP-K (Ac)-N-3Pya-Sar-CONH2,
MeCO-k (PEG2PEG2C18OH)-Pen-N-T-7MeW-K (Ac)-Pen-AEF-2Nal-THP-K (Ac)-N-3Pya-Sar-CONH2,
MeCO-r-Pen-K (PEG6gEC18OH)-T-7MeW-K (Ac)-Pen-AEF-2Nal-THP-K (Ac)-N-3Pya-Sar-CONH2,
MeCO-r-Pen-K (PEG2PEG6gEC18OH)-T-7MeW-K (Ac)-Pen-AEF-2Nal-THP-K (Ac)-N -3Pya-Sar-CONH2,
MeCO-r-Pen-N-T-7MeW-K (Ac)-Pen-AEF-2Nal-THP-K (PEG2PEG2-pppC18OH)-N-3Pya-Sar-CONH2,
MeCO-k (gEC16)-Pen-N-T-7MeW-K (Ac)-Pen-AEF-2Nal-THP-K (Ac)-N-3Pya-Sar-CONH2,
MeCO-k (gEC18)-Pen-N-T-7MeW-K (Ac)-Pen-AEF-2Nal-THP-K (Ac)-N-3Pya-Sar-CONH2,
MeCO-r-Pen-K (gEC16)-T-7MeW-K (Ac)-Pen-AEF-2Nal-THP-K (Ac)-N-3Pya-Sar-CONH2,
MeCO-r-Pen-K (gEC18)-T-7MeW-K (Ac)-Pen-AEF-2Nal-THP-K (Ac)-N-3Pya-Sar-CONH2,
MeCO-k (PEG2PEG2gETrxC18OH)-Pen-N-T-7MeW-K (Ac)-Pen-AEF-2Nal-THP-K (Ac)-N-3Pya-Sar-CONH2,
MeCO-k (PEG2PEG2gETrxC20OH)-Pen-N-T-7MeW-K (Ac)-Pen-AEF-2Nal-THP-K (Ac)-N-3Pya-Sar-CONH2,
MeCO-Pen-K (PEG2PEG2gETrxC18OH)-T-7MeW-K (Ac)-Pen-AEF-2Nal-THP-K (Ac)-N -3Pya-Sar-CONH2,
MeCO-Pen-K (PEG2PEG2gETrxC20OH)-T-7MeW-K (Ac)-Pen-AEF-2Nal-THP-K (Ac)-N -3Pya-Sar-CONH2,
MeCO-Pen-N-T-7MeW-K (Ac)-Pen-AEF-2Nal-THP-K (gEC16)-N-3Pya-Sar-CONH2,
MeCO-k (PEG2PEG2gEC16OH)-Pen-K (PEG2PEG2gEC16OH)-T-7MeW-K (Ac)-Pen-AEF -2Nal-THP-K (Ac)-N-3Pya-Sar-CONH2,
MeCO-k (PEG2PEG2gEC18OH)-Pen-K (PEG2PEG2gEC18OH)-T-7MeW-K (Ac)-Pen-AEF -2Nal-THP-K (Ac)-N-3Pya-Sar-CONH2,
MeCO-k (PEG2PEG2gEC20OH)-Pen-K (PEG2PEG2gEC20OH)-T-7MeW-K (Ac)-Pen-AEF -2Nal-THP-K (Ac)-N-3Pya-Sar-CONH2,
MeCO-k (gEC16)-Pen-K (gEC16)-T-7MeW-K (Ac)-Pen-AEF-2Nal-THP-K (Ac)-N-3Pya-Sar-CONH2,
MeCO-k (gEC18)-Pen-K (gEC18)-T-7MeW-K (Ac)-Pen-AEF-2Nal-THP-K (Ac)-N-3Pya-Sar-CONH2,
MeCO-k (PEG2PEG2gEC16)-Pen-N-T-7MeW-K (Ac)-Pen-AEF-2Nal-THP-K (Ac)-N-3Pya-Sar-CONH2,
MeCO-k (PEG2PEG2gEC18)-Pen-N-T-7MeW-K (Ac)-Pen-AEF-2Nal-THP-K (Ac)-N-3Pya-Sar-CONH2,
MeCO-Pen-K (PEG2PEG2gEC16)-T-7MeW-K (Ac)-Pen-AEF-2Nal-THP-K (Ac)-N-3Pya-Sar-CONH2,
MeCO-Pen-K (PEG2PEG2gEC18)-T-7MeW-K (Ac)-Pen-AEF-2Nal-THP-K (Ac)-N-3Pya-Sar-CONH2,
MeCO-Pen-K (1PEG21PEG2IsoGluC16Diacid)-T-7MeW-K (Ac)-Pen-AEF-2Nal-THP-K (Ac)-N-3Pya-Sar-CONH2,
MeCO-r-Pen-N-T-7MeW-K (PEG2PEG2gEC18OH)-Pen-AEF-2Nal-THP-K (Ac)-N-3Pya-Sar-CONH2,
MeCO-Pen-N-T-7MeW-K (Ac)-Pen-AEF-2Nal-THP-K (gEC20OH)-N-3Pya-Sar-CONH2,
MeCO-Pen-N-T-7MeW-K (Ac)-Pen-AEF-2Nal-THP-K (PEG2PEG2-TrxgEC18OH)-N-3Pya-Sar-CONH2,
MeCO-Pen-N-T-7MeW-K (Ac)-Pen-AEF-2Nal-THP-K (PEG2PEG2-TrxgETrxC20OH)-N-3Pya-Sar-CONH2,
MeCO-Pen-N-T-7MeW-K (Ac)-Pen-AEF-2Nal-THP-K (PEG2PEG2-TrxgEC20OH)-N-3Pya-Sar-CONH2,
MeCO-k (PEG2PEG2gEC10OH)-Pen-N-T-7MeW-K (Ac)-Pen-AEF-2Nal-THP-K (gEC16)-N-3Pya-Sar-CONH2,
MeCO-k (PEG2PEG2gEC18OH)-Pen-K (gEC16)-T-7MeW-K (Ac)-Pen-AEF-2Nal-THP-K (Ac)-N-3Pya-Sar-CONH2,
MeCO-r-Pen-K (PEG2PEG2pgEC18OH)-T-7MeW-K (Ac)-Pen-AEF-2Nal-THP-K (Ac)-N -3Pya-Sar-CONH2,
MeCO-Pen-N-T-7MeW-K (Ac)-Pen-AEF-2Nal-THP-K (PEG2PEG2gEmXOH)-N-3Pya-Sar-CONH2,
MeCO-k (PEG2PEG2gEC18OH)-Pen-N-T-7MeW-K (Ac)-Pen-AEF-2Nal-THP-K (gEC16)-N-3Pya-Sar-CONH2,
MeCO-r-Pen-K (PEG2PEG2pppgEC18OH)-T-7MeW-K (Ac)-Pen-AEF-2Nal-THP-K (Ac)-N-3Pya-Sar-CONH2,
MeCO-k (PEG2PEG2PgEC18OH)-Pen-N-T-7MeW-K (Ac)-Pen-AEF-2Nal-THP-K (Ac)-N -3Pya-Sar-CONH2,
MeCO-k (PEG2PEG2PPPgEC18OH)-Pen-N-T-7MeW-K (Ac)-Pen-AEF-2Nal-THP-K (Ac)-N-3Pya-Sar-CONH2,
MeCO-Pen-N-T-7MeW-K (Ac)-Pen-AEF-2Nal-THP-K (PEG2PEG2PgEC18OH)-N -3Pya-Sar-CONH2,
MeCO-Pen-N-T-7MeW-K (Ac)-Pen-AEF-2Nal-THP-K (PEG2PEG2PPPgEC18OH)-N -3Pya-Sar-CONH2,
MeCO-k (PEG2PEG2-TrxgEC18OH)-Pen-N-T-7MeW-K (Ac)-Pen-AEF-2Nal-THP-K (Ac)-N-3Pya-Sar-CONH2,
MeCO-k (PEG2PEG2gEmXOH)-Pen-N-T-7MeW-K (Ac)-Pen-AEF-2Nal-THP-K (Ac)-N -3Pya-Sar-CONH2,
MeCO-Pen-N-T-7MeW-K (Ac)-Pen-AEF-2Nal-THP-K (PEG2PEG2gEpXOH)-N-3Pya-Sar-CONH2,
MeCO-k (PEG2PEG2gEpXOH)-Pen-N-T-7MeW-K (Ac)-Pen-AEF-2Nal-THP-K (PEG2PEG2gEpXOH)-N-3Pya-Sar-CONH2,
MeCO-k (PEG2PEG2gEmXOH)-Pen-N-T-7MeW-K (Ac)-Pen-AEF-2Nal-THP-K (gEC16)-N-3Pya-Sar-CONH2,
MeCO-Pen-N-T-7MeW-K (Ac)-Pen-AEF-2Nal-THP-K (DAP (C16OH) 2)-N-3Pya-Sar-CONH2,
MeCO-k (DAP (C16OH) 2)-Pen-N-T-7MeW-K (Ac)-Pen-AEF-2Nal-THP-K (Ac)-N-3Pya-Sar-CONH2,
MeCO-Pen-N-T-7MeW-K (Ac)-Pen-AEF-2Nal-THP-K (PEG2PEG2gEDAP (C16OH) 2)-N -3Pya-Sar-CONH2,
MeCO-k (PEG2PEG2gEDAP (C16OH) 2)-Pen-N-T-7MeW-K (Ac)-Pen-AEF-2Nal-THP-K (Ac)-N-3Pya-Sar-CONH2,
MeCO-Pen-N-T-7MeW-K (Ac)-Pen-AEF-2Nal-THP-K (PEG2PEG2Sp6gEC18OH)-N -3Pya-Sar-CONH2,
MeCO-Pen-N-T-7MeW-K (Ac)-Pen-AEF-2Nal-THP-K (PEG2Sp6PEG2gEC18OH)-N -3Pya-Sar-CONH2,
MeCO-k (PEG2PEG2gEC16OH)-Pen-N-T-7MeW-K (Ac)-Pen-AEF-2Nal-THP-K (gEC18)-N-3Pya-Sar-CONH2,
MeCO-k (PEG2PEG2gEC18OH)-Pen-N-T-7MeW-K (Ac)-Pen-AEF-2Nal-THP-K (gEC18)-N-3Pya-Sar-CONH2,
MeCO-Pen-K (1PEG21PEG2IsoGluC18Diacid)-T-7MeW-K (Ac)-Pen-AEF-2Nal-THP-K (Ac)-N-3Pya-Sar-CONH2,
MeCO-Pen-K (PEG2PEG2gEC18OH)-T-7MeW-K (Ac)-Pen-AEF-2Nal-THP-K (PEG2PEG2gESp6C18OH)-N-3Pya-Sar-CONH2,
MeCO-Pen-K (PEG2PEG2gEC18OH)-T-7MeW-K (Ac)-Pen-AEF-2Nal-THP-K (Sp6PEG2PEG2gEC18OH)-N-3Pya-Sar-CONH2,
MeCO-k (gEC16)-Pen-N-T-7MeW-K (Ac)-Pen-AEF-2Nal-THP-K (gEC18)-N-3Pya-Sar-CONH2,
MeCO-k (gEC18)-Pen-N-T-7MeW-K (Ac)-Pen-AEF-2Nal-THP-K (gEC18)-N-3Pya-Sar-CONH2,
MeCO-k (PEG2PEG2gEC10OH)-Pen-N-T-7MeW-K (Ac)-Pen-AEF-2Nal-THP-K (gEC18)-N-3Pya-Sar-CONH2,
MeCO-Pen-K (PEG2PEG2gEC18OH)-T-7MeW-K (Ac)-Pen-AEF-2Nal-THP-K (PEG2PEG2gETrxC20OH)-N-3Pya-Sar-CONH2,
MeCO-k (PEG2PEG2gESp6C18OH)-Pen-N-T-7MeW-K (Ac)-Pen-AEF-2Nal-THP-K (Ac)-N-3Pya-Sar-CONH2,
MeCO-k (PEG2Sp6PEG2gEC18OH)-Pen-N-T-7MeW-K (Ac)-Pen-AEF-2Nal-THP-K (Ac)-N-3Pya-Sar-CONH2,
MeCO-k (Sp6PEG2PEG2gEC18OH)-Pen-N-T-7MeW-K (Ac)-Pen-AEF-2Nal-THP-K (Ac)-N-3Pya-Sar-CONH2,
MeCO-K [PEG2PEG2-gEDAP (pXOH) 2] -Pen-N-T-7MeW-K (Ac)-Pen-AEF-2Nal-THP-K (Ac)-N-3Pya-Sar-CONH2,
MeCO-Pen-N-T-7MeW-K (Ac)-Pen-AEF-2Nal-THP-K [PEG2PEG2-gEDAP-(mXOH) 2] -N-3Pya-Sar-CONH2,
MeCO-k (GolAC16)-Pen-N-T-7MeW-K (Ac)-Pen-AEF-2Nal-THP-K (Ac)-N-3Pya-Sar-CONH2,
MeCO-k (GolAC16OH)-Pen-N-T-7MeW-K (Ac)-Pen-AEF-2Nal-THP-K (Ac)-N-3Pya-Sar-CONH2,
MeCO-k (GolAC18OH)-Pen-N-T-7MeW-K (Ac)-Pen-AEF-2Nal-THP-K (Ac)-N-3Pya-Sar-CONH2,
MeCO-k (PEG2PEG2 GolAC18OH)-Pen-N-T-7MeW-K (Ac)-Pen-AEF-2Nal-THP-K (Ac)-N-3Pya-Sar-CONH2,
MeCO-k (PEG2PEG2-C18GolB)-Pen-N-T-7MeW-K (Ac)-Pen-AEF-2Nal-THP-K (Ac)-N -3Pya-Sar-CONH2,
MeCO-k (PEG2PEG2gE (C) C18OH)-Pen-N-T-7MeW-K (Ac)-Pen-AEF-2Nal-THP-K (Ac)-N-3Pya-Sar-CONH2,
MeCO-k (PEG2PEG2gEC18OH (C))-Pen-N-T-7MeW-K (Ac)-Pen-AEF-2Nal-THP-K (Ac)-N-3Pya-Sar-CONH2,
MeCO-Pen-N-T-7MeW-K (Ac)-Pen-AEF-2Nal-THP-E-N-3Pya-Sar-k (PEG12_IsoGlu_C18_Diacid)-CONH2,
MeCO-Pen-N-T-7MeW-K (Ac)-Pen-AEF-2Nal-THP-E-N-3Pya-Sar-k (Peg4_IsoGlu_Palm)-CONH2,
MeCO-Pen-N-T-7MeW-K (Ac)-Pen-AEF-2Nal-THP-E-N-3Pya-Sar-k (IsoGlu_Palm)-CONH2,
MeCO-Pen-N-T-7MeW-K (Ac)-Pen-AEF-2Nal-THP-E-N-3Pya-Sar-k (PEG12_C18_Diacid)-CONH2,
MeCO-Pen-N-T-7MeW-K (Ac)-Pen-AEF-2Nal-THP-E-N-3Pya-Sar-k (Peg4_IsoGlu_C18_Diacid)-CONH2,
MeCO-Pen-N-T-7MeW-K (Ac)-Pen-AEF-2Nal-THP-E-N-3Pya-Sar-k (PEG12_IsoGlu_Palm)-CONH2,
MeCO-Pen-N-T-7MeW-K (Ac)-Pen-AEF-2Nal-THP-E-N-3Pya-Sar-k (Peg4_C18_Diacid)-CONH2,
MeCO-Pen-N-T-7MeW-K (Ac)-Pen-AEF-2Nal-THP-E-N-3Pya-Sar-k (IsoGlu_C18_Diacid)-CONH2,
MeCO-r-Pen-K (1PEG21PEG2IsoGluC16Diacid)-T-7MeW-K (Ac)-Pen-AEF-2Nal-THP-E -N-3Pya-Sar-CONH2,
MeCO-r-Pen-K (1PEG21PEG2IsoGluC18Diacid)-T-7MeW-K (Ac)-Pen-AEF-2Nal-THP-E -N-3Pya-Sar-CONH2,
MeCO-Pen-N-T-7MeW-K (Ac)-Pen-AEF-2Nal-THP-K (1PEG21PEG2IsoGluC16Diacid)-N-3Pya-Sar-CONH2,
MeCO-Pen-N-T-7MeW-K (Ac)-Pen-AEF-2Nal-THP-K (1PEG21PEG2IsoGluC18Diacid)-N-3Pya-Sar-CONH2,
MeCO-r-Pen-N-T-7MeW-K (Ac)-Pen-AEF-2Nal-THP-E-N-3Pya-K (1PEG21PEG2IsoGluC18Diacid)-CONH2,
MeCO-r-Pen-N-T-7MeW-K (Ac)-Pen-AEF-2Nal-THP-E-N-3Pya-Sar-K (1PEG21PEG2IsoGluC16Diacid)-CONH2,
MeCO-r-Pen-N-T-7MeW-K (Ac)-Pen-AEF-2Nal-THP-E-N-3Pya-Sar-K (1PEG21PEG2IsoGluC18Diacid)-CONH2,
HOC10gEPEG2COPEG2CO-r-Pen-N-T-7MeW-K (Ac)-Pen-AEF-2Nal-THP-K (PEG2PEG2gEC10OH)-N-3Pya-Sar-CONH2,
MeCO-Pen-N-T-7MeW-K (Ac)-Pen-AEF-2Nal-THP-K (PEG2PEG2gEC10OH)-N-3Pya-Sar-CONH2,
MeCO-k (PEG2PEG2gEC10OH)-Pen-N-T-7MeW-K (Ac)-Pen-AEF-2Nal-THP-K (Ac)-N -3Pya-Sar-CONH2,
MeCO-Pen-K (PEG2PEG2gEC10OH)-T-7MeW-K (Ac)-Pen-AEF-2Nal-THP-K (Ac)-N -3Pya-Sar-CONH2,
MeCO-Pen-N-T-7MeW-K (Ac)-Pen-AEF-2Nal-THP-K (gEC10OH)-N-3Pya-Sar-CONH2,
MeCO-k (PEG2PEG2gEC10OH)-Pen-K (PEG2PEG2gEC10OH)-T-7MeW-K (Ac)-Pen-AEF -2Nal-THP-K (Ac)-N-3Pya-Sar-CONH2,
HOC16gEPEG2PEG2orn (2)-r-Pen-N-T-7MeW-K (Ac)-Pen-AEF-2Nal-THP-E (2)-N -3Pya-Sar-CONH2,
MeCO-k (PEG2PEG2gEC12OH (c))-Pen-N-T-7MeW-K (Ac)-Pen-AEF-2Nal-THP-K (Ac)-N-3Pya-Sar-CONH2,
MeCO-k (PEG2PEG2gE (C) C12)-Pen-N-T-7MeW-K (Ac)-Pen-AEF-2Nal-THP-K (Ac)-N -3Pya-Sar-CONH2,
MeCO-r-Pen-N-T-7MeW-K (Ac)-Pen-AEF-2Nal-THP-K (FITCPEG4)-N-3Pya-Sar-K (PEG2PEG2gEC18OH)-CONH2,
succiniccarn-k (PEG2PEG2gEC18OH)-Pen-N-T-7MeW-K (Ac)-Pen-AEF-2Nal-THP-E-N -3Pya-Sar-CONH2,
MeCO-r-Pen-K (PEG2PEG2gEC18OH)-T-7MeW-K (Ac)-Pen-dAEF-2Nal-THP-E-N-3Pya-Sar-CONH2,
MeCO-Pen-N-T-W-K (PEG2PEG2gEC20OH)-Pen-AEF-2Nal-THP-K (Ac)-N-3Pya-Sar-CONH2,
MeCO-Pen-K (PEG2PEG2gEC20OH)-T-W-K (Ac)-Pen-AEF-2Nal-THP-K (Ac)-N-3Pya-Sar-CONH2,
MeCO-K (PEG2PEG2gEC20OH)-Pen-N-T-W-K (Ac)-Pen-AEF-2Nal-THP-K (Ac)-N-3Pya-Sar-CONH2,
MeCO-Pen-N-T-W-K (Ac)-Pen-AEF-2Nal-THP-K (Ac)-N-3Pya-Sar-K (PEG2PEG2gEC20OH)-CONH2,
MeCO-r-Pen-N-T-7MeW-K (PEG2PEG2gEC20OH)-Pen-AEF-2Nal-THP-E-N-3Pya-Sar-CONH2,
cPEG3aCO-r-Pen-N-T-7MeW-K (PEG2PEG2gEC20OH)-Pen-AEF-2Nal-THP-E-N-3Pya-Sar-CONH2,
cPEG3aCO-r-Pen-K (PEG2PEG2gEC20OH)-T-7MeW-K (Ac)-Pen-AEF-2Nal-THP-E-N-3Pya-Sar-CONH2,
MeCO-r-Pen-N-T-7MeW-K (Ac)-Pen-AEF-2Nal-THP-E-N-3Pya-Sar-K (PEG2PEG2gEC20OH)-CONH2,
MeCO-r-Abu-N-T-7MeW-K (Ac)-aMeC-AEF-2Nal-THP-E-N-3Pya-Sar-K (PEG2PEG2gEC18OH)-CONH2,
MeCO-r-Abu-N-T-7MeW-K (Ac)-Pen-AEF-2Nal-THP-E-N-3Pya-Sar-K (PEG2PEG2gEC18OH)-CONH2,
MeCO-Pen-N-T-7MeW-K (PEG2PEG2gEC12)-Pen-AEF-2Nal-THP-K (PEG2PEG2gEC12)-N-3Pya-Sar-CONH2,
MeCO-Pen-N-T-7MeW-K (PEG2PEG2gEC14)-Pen-AEF-2Nal-THP-K (PEG2PEG2gEC14)-N-3Pya-Sar-CONH2,
MeCO-k (PEG2PEG2gEC12)-Pen-N-T-7MeW-K (Ac)-Pen-AEF-2Nal-THP-E-N-3Pya-Sar-CONH2,
MeCO-k (PEG2PEG2gEC14)-Pen-N-T-7MeW-K (Ac)-Pen-AEF-2Nal-THP-E-N-3Pya-Sar-CONH2,
MeCO-r-Pen-N-T-7MeW-K (PEG2PEG2gEC12)-Pen-AEF-2Nal-THP-E-N-3Pya-Sar-K (PEG2PEG2gEC12)-CONH2,
MeCO-r-Pen-N-T-7MeW-K (PEG2PEG2gEC12)-Pen-AEF-2Nal-THP-K (PEG2PEG2gEC12)-N-3Pya-Sar-CONH2,
MeCO-r-Pen-N-T-7MeW-K (Ac)-Pen-AEF-2Nal-THP-K (PEG2PEG2gEC12)-N-3Pya-Sar-CONH2,
MeCO-r-Pen-N-T-7MeW-K (PEG2PEG2gEC12)-Pen-AEF-2Nal-THP-E-N-3Pya-Sar-CONH2,
MeCO-r-Pen-N-T-7MeW-K (Ac)-Pen-AEF-2Nal-THP-E-N-3Pya-Sar-K (PEG2PEG2gEC12)-CONH2,
cPEG3aCO-k (PEG2PEG2PEG2PEG2gEC12)-Pen-N (N (Me) 2)-T-7MeW-K (Ac)-Pen-TMAPF -2Nal-THP-K (NMeAc)-N-3Pya-NMeK (PEG2PEG2PEG2PEG2gEC12)-CONH2,
MeCO-Pen-N-T-7MeW-K (PEG2PEG2gEC14)-Pen-AEF-2Nal-THP-E-N-3Pya-Sar-CONH2,
MeCO-Pen-K (PEG2PEG2gEC14)-T-7MeW-K (Ac)-Pen-AEF-2Nal-THP-K (Ac)-N-3Pya-Sar-CONH2,
MeCO-Pen-N-T-7MeW-K (PEG2PEG2gEC12)-Pen-AEF-2Nal-THP-K (Ac)-N-3Pya-Sar-CONH2,
MeCO-Pen-N-T-7MeW-K (Ac)-Pen-AEF-2Nal-THP-K (PEG2PEG2gEC14)-N-3Pya-Sar-CONH2,
MeCO-Pen-K (PEG2PEG2gEC12)-T-7MeW-K (Ac)-Pen-AEF-2Nal-THP-K (Ac)-N-3Pya-Sar-CONH2,
MeCO-Pen-N-T-7MeW-K (PEG2PEG2gEC14)-Pen-AEF-2Nal-THP-K (Ac)-N-3Pya-Sar-CONH2,
MeCO-Pen-N-T-7MeW-K (Ac)-Pen-AEF-2Nal-THP-K (PEG2PEG2gEC12)-N-3Pya-Sar-CONH2,
MeCO-r-Abu-N-T-7MeW-K (Ac)-aMeC-AEF-2Nal-THP-K (PEG2PEG2gEC12)-N-3Pya-Sar-CONH2,
MeCO-Pen-K (PEG2PEG2gEC14)-T-7MeW-K (Ac)-Pen-AEF-2Nal-THP-E-N-3Pya-Sar-CONH2,
MeCO-Pen-N-T-7MeW-K (PEG2PEG2gEC12)-Pen-AEF-2Nal-THP-E-N-3Pya-Sar-CONH2,
MeCO-r-Abu-N-T-W-K (Ac)-aMeC-AEF-2Nal-THP-K (PEG2PEG2gEC12)-N-3Pya-Sar-CONH2,
MeCO-r-Abu-N-T-W-K (Ac)-aMeC-AEF-2Nal-THP-E-N-3Pya-Sar-K (PEG2PEG2gEC12)-CONH2,
MeCO-r-Abu-N-T-7MeW-K (PEG2PEG2gEC12)-aMeC-AEF-2Nal-THP-E-N-3Pya-Sar-CONH2,
MeCO-r-Abu-N-T-7MeW-K (Ac)-aMeC-AEF-2Nal-THP-E-N-3Pya-Sar-K (PEG2PEG2gEC12)-CONH2,
MeCO-r-Abu-N-T-W-K (PEG2PEG2gEC12)-aMeC-AEF-2Nal-THP-E-N-3Pya-Sar-CONH2,
MeCO-Pen-N-T-7MeW-K (Ac)-Pen-AEF (G)-2Nal-THP-K (PEG2PEG2gEC14)-N-3Pya-Sar-CONH2,
MeCO-Pen-N-T-7MeW-K (Ac)-Pen-AEF-2Nal-THP-E-N-3Pya-Sar-K (PEG2PEG2gEC12)-CONH2,
MeCO-Pen-N-T-7MeW-K (Ac)-Pen-AEF-2Nal-THP-E-N-3Pya-Sar-K (PEG2PEG2gEC14)-CONH2,
MeCO-Pen-K (PEG2PEG2gEC12)-T-7MeW-K (Ac)-Pen-AEF-2Nal-THP-E-N-3Pya-Sar-CONH2,
MeCO-r-Pen-N-T-7MeW-K (Ac)-Pen-AEF-2Nal-THP-E-N-3Pya-Sar-K (PEG2NMePEG2NMegENMeC18Tetrazole)-CONH2,
MeCO-Pen-K (C14)-T-7MeW-K (Ac)-Pen-AEF-2Nal-THP-E-N-3Pya-Sar-CONH2,
MeCO-Pen-N-T-7MeW-K (C14)-Pen-AEF-2Nal-THP-E-N-3Pya-Sar-CONH2,
MeCO-Pen-N-T-7MeW-K (Ac)-Pen-AEF-2Nal-THP-K (C14)-N-3Pya-Sar-CONH2,
MeCO-Pen-K (gEC14)-T-7MeW-K (Ac)-Pen-AEF-2Nal-THP-E-N-3Pya-Sar-CONH2,
MeCO-Pen-N-T-7MeW-K (gEC14)-Pen-AEF-2Nal-THP-E-N-3Pya-Sar-CONH2,
MeCO-Pen-N-T-7MeW-K (Ac)-Pen-AEF-2Nal-THP-K (gEC14)-N-3Pya-Sar-CONH2,
MeCO-Pen-N-T-7MeW-K (Ac)-Pen-AEF-2Nal-THP-K (Ac)-N-3Pya-Sar-K (PEG2PEG2gEC14)-CONH2,
MeCO-Pen-N-T-7MeW-K (Ac)-Pen-AEF-2Nal-THP-E-N-3Pya-Sar-K (gEC14)-CONH2,
MeCO-Pen-N-T-7MeW-K (Ac)-Pen-AEF-2Nal-THP-E-N-3Pya-Sar-K (gEC12)-CONH2,
MeCO-k (PEG2PEG2gEC12)-Pen-N-T-7MeW-K (Ac)-Pen-AEF-2Nal-THP-K (Ac)-N -3Pya-Sar-CONH2,
MeCO-k (PEG2PEG2gEC14)-Pen-N-T-7MeW-K (Ac)-Pen-AEF-2Nal-THP-K (Ac)-N -3Pya-Sar-CONH2,
MeCO-Pen-N-T-7MeW-K (Ac)-Pen-AEF-2Nal-THP-E-N-3Pya-Sar-K (C14)-CONH2,
MeCO-Pen-N-T-7MeW-K (Ac)-Pen-AEF-2Nal-THP-K (Ac)-N-3Pya-Sar-K (PEG2PEG2gEC12)-CONH2,
MeCO-Pen-N-T-7MeW-K (Ac)-Pen-AEF-2Nal-THP-E-N-3Pya-Sar-K (PEG2PEG2gEDProC14)-CONH2,
MeCO-Pen-N-T-7MeW-K (Ac)-Pen-AEF-2Nal-THP-E-N-3Pya-Sar-K (PEG2PEG2C14)-CONH2,
MeCO-Pen-N-T-7MeW-K (Ac)-Pen-AEF-2Nal-THP-E-N-3Pya-Sar-K (GSGSGSGC14)-CONH2,
MeCO-Pen-N-T-7MeW-K (Ac)-Pen-AEF-2Nal-THP-E-N-3Pya-Sar-K (PEG2PEG2SP6C14)-CONH2,
MeCO-Pen-N-T-7MeW-K (Ac)-Pen-AEF-2Nal-THP-E-N-3Pya-Sar-K (PEG2C14)-CONH2,
MeCO-Pen-N-T-7MeW-K (Ac)-Pen-AEF-2Nal-THP-E-N-3Pya-Sar-K (PEG2PEG2gESarC14)-CONH2, and
MeCO-Pen-N-T-7MeW-K (Ac)-Pen-AEF-2Nal-THP-E-N-3Pya-Sar-K (PEG2PEG2gEProC14)-CONH2,
PEG2PEG2-gEC16OH-r-Pen-N-T-7MeW-K (Ac)-Pen-AEF-2Nal-THP-K (Ac)-N-3Pya-Sar-CONH2,
PEG2 Palm-Pen-N-T-7MeW-K (Ac)-Pen-AEF-2Nal-THP-E-N-3Pya-Sar-CONH2,
PEG2_Lauryl-Pen-N-T-7MeW-K (Ac)-Pen-AEF-2Nal-THP-E-N-3Pya-Sar-CONH2,
PEG4 Palm-Pen-N-T-7MeW-K (Ac)-Pen-AEF-2Nal-THP-E-N-3Pya-Sar-CONH2,
PEG4_Lauryl-Pen-N-T-7MeW-K (Ac)-Pen-AEF-2Nal-THP-E-N-3Pya-Sar-CONH2,
PEG4_Capryl-Pen-N-T-7MeW-K (Ac)-Pen-AEF-2Nal-THP-E-N-3Pya-Sar-CONH2,
PEG4_Hexyl-Pen-N-T-7MeW-K (Ac)-Pen-AEF-2Nal-THP-E-N-3Pya-Sar-CONH2,
C12gEPEG2PEG2CO-Pen-N-T-7MeW-K (PEG2PEG2gEC12)-Pen-AEF-2Nal-THP-E-N -3Pya-Sar-CONH2,
C14gEPEG2PEG2CO-Pen-N-T-7MeW-K (PEG2PEG2gEC14)-Pen-AEF-2Nal-THP-E-N -3Pya-Sar-CONH2,
C14gEPEG2PEG2CO-k (PEG2PEG2gEC14)-Pen-N-T-7MeW-K (Ac)-Pen-AEF-2Nal-THP -E-N-3Pya-Sar-CONH2,
C12gEPEG2PEG2CO-k (PEG2PEG2gEC12)-Pen-N-T-7MeW-K (Ac)-Pen-AEF-2Nal-THP -E-N-3Pya-Sar-CONH2,
C14gEPEG2PEG2CO-Pen-N-T-7MeW-K (Ac)-Pen-AEF-2Nal-THP-E-N-3Pya-Sar-K (PEG2PEG2gEC14)-CONH2,
C12gEPEG2PEG2CO-Pen-N-T-7MeW-K (Ac)-Pen-AEF-2Nal-THP-E-N-3Pya-Sar-CONH2,
C14gEPEG2PEG2CO-Pen-N-T-7MeW-K (Ac)-Pen-AEF-2Nal-THP-E-N-3Pya-Sar-CONH2,
C12gEPEG2PEG2CO-Pen-K (PEG2PEG2gEC12)-T-7MeW-K (Ac)-Pen-AEF-2Nal-THP -E-N-3Pya-Sar-CONH2,
C14gEPEG2PEG2CO-Pen-K (PEG2PEG2gEC14)-T-7MeW-K (Ac)-Pen-AEF-2Nal-THP -E-N-3Pya-Sar-CONH2,
C12gEPEG2PEG2CO-Pen-N-T-7MeW-K (Ac)-Pen-AEF-2Nal-THP-K (PEG2PEG2gEC12)-N-3Pya-Sar-CONH2,
C14gEPEG2PEG2CO-Pen-N-T-7MeW-K (Ac)-Pen-AEF-2Nal-THP-K (PEG2PEG2gEC14)-N-3Pya-Sar-CONH2,
C12gEPEG2PEG2CO-r-Pen-N-T-7MeW-K (Ac)-Pen-AEF-2Nal-THP-E-N-3Pya-Sar-CONH2,
C12gEPEG2PEG2CO-r-Pen-N-T-7MeW-K (Ac)-Pen-AEF-2Nal-THP-E-N-3Pya-Sar-K (PEG2PEG2gEC12)-CONH2,
C12gEPEG2PEG2CO-r-Pen-N-T-7MeW-K (Ac)-Pen-AEF-2Nal-THP-K (PEG2PEG2gEC12)-N-3Pya-Sar-CONH2,
C12gEPEG2PEG2CO-Pen-N-T-7MeW-K (Ac)-Pen-AEF (G)-2Nal-THP-E-N-3Pya-Sar-CONH2,
C12gEPEG2PEG2CO-r-Pen-N-T-7MeW-K (PEG2PEG2gEC12)-Pen-AEF-2Nal-THP-E-N -3Pya-Sar-CONH2,
C12gEPEG2PEG2CO-Pen-N-T-7MeW-K (Ac)-Pen-AEF-2Nal-THP-K (Ac)-N-3Pya-Sar-CONH2, and
C14gEPEG2PEG2CO-Pen-N-T-7MeW-K (Ac)-Pen-AEF-2Nal-THP-K (Ac)-N-3Pya-Sar-CONH2.

In some embodiments, the present disclosure provides a peptide of Formula (I), comprising the amino acid sequence:

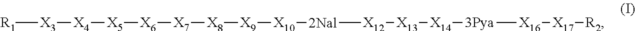

or a pharmaceutically acceptable salt thereof, wherein:
R₁ is an N-terminal capping group (e.g., MeCO), Z_{peg}, or Z_{lipid};
X₃ is any amino acid or absent;
X₄ is 4AminoPro, Abu, aG, aMeC, C, Dap, Pen, Pen(oXyl), Pen(mXyl), Pen(pXyl), or Pra;
X₅ is any amino acid;
X₆ is any amino acid;
X₇ is 7MeW, W, or absent;
X₈ is any amino acid;
X₉ is aMeC, aG, C, D, E, hE, Pen, Dap, or Dap(N3)
X₁₀ is AEF, TMAPF, AEF(d), TMAPF-Z_{lipid}, APEG3F, or AEF-Z_{lipid};
X₁₂ is any amino acid;
X₁₃ is any amino acid;
X₁₄ is any amino acid;
X₁₆ is any amino acid;
X₁₇ is any amino acid or absent;
R₂ is a C-terminal capping group (e.g., CONH2), CONH—Z_{peg}, or CO—Z_{lipid};
Z_{peg}, independently for each occurrence, is a polyethylene glycol chain;
Z_{lipid}, independently for each occurrence, is a lipophilic substituent;
wherein the peptide comprises a linkage between the residues at X₄ and X₉; and
wherein the polypeptide comprises at least one lipophilic substituent at a position selected from R₁, X₃, X₅, X₆, X₈, X₁₀, X₁₂, X₁₃, X₁₄, X₁₆, X₁₇, or R₂;
provided that the polypeptide is not selected from the group consisting of:
MeCO-r-Pen-N-T-7MeW-K (Ac)-Pen-AEF-2Nal-THP-E-N-3Pya-Sar-K (PEG2PEG2gEC18OH)-CONH2,
MeCO-r-Pen-N-T-7MeW-K (Ac)-Pen-AEF-2Nal-THP-E-N-3Pya-NMeK (PEG2PEG2gEC18OH)-CONH2,
MeCO-Pen-N-T-7MeW-K (Ac)-Pen-AEF-2Nal-THP-K (PEG2PEG2gEC18OH)-N-3Pya-Sar-CONH2,
MeCO-k (PEG2PEG2gEC18OH)-Pen-N-T-7MeW-K (Ac)-Pen-AEF-2Nal-THP-K (Ac)-N -3Pya-Sar-CONH2,
MeCO-r-Pen-N-T-7MeW-K (Ac)-Pen-AEF (PEG2PEG2gEC18OH)-2Nal-THP-K (Ac)-N -3Pya-Sar-CONH2,
MeCO-k (PEG2PEG2SP6gEC18OH)-Pen-N-T-7MeW-K (Ac)-Pen-AEF-2Nal-THP-K (Ac)-N-3Pya-Sar-CONH2,
MeCO-Pen-N-T-7MeW-K (Ac)-Pen-AEF-2Nal-THP-K (Ac)-N-3Pya-Sar-PEG2-PEG2,
MeCO-k (PEG2PEG2gEmXOH)-Pen-N-T-7MeW-K (Ac)-Pen-AEF-2Nal-THP-K (PEG2PEG2gEmXOH)-N-3Pya-Sar-CONH2,
MeCO-k (PEG2PEG2gEC16OH)-Pen-N-T-7MeW-K (Ac)-Pen-AEF-2Nal-THP-K (gEC16)-N-3Pya-Sar-CONH2,
HOC18gEPEG2PEG2-r-Pen-N-T-7MeW-K (Ac)-Pen-AEF-2Nal-THP-K (Ac)-N-3Pya-Sar-CONH2,
HOC18gEPEG2PEG2-r-Pen-N-T-7MeW-K (Ac)-Pen-AEF-2Nal-THP-K (PEG2PEG2gEC18OH)-N-3Pya-Sar-CONH2,
MeCO-k (PEG2PEG2gEC18OH)-Pen-N-T-7MeW-K (Ac)-Pen-AEF-2Nal-THP-K (Ac)-N -3Pya-N (4AmBenzyl) Gly-CONH2,
MeCO-r-Pen-N-T-7MeW-K (Ac)-Pen-AEF-2Nal-THP-K (Ac)-N-3Pya-N (PEG2PEG2gEC18OH) Gly-CONH2,
MeCO-k (PEG2PEG2gEDab (mXOH) 2)-Pen-N-T-7MeW-K (Ac)-Pen-AEF-2Nal-THP-K (Ac)-N-3Pya-Sar-CONH2,
MeCO-k (PEG2PEG2gE (c) C18OH)-Pen-N-T-7MeW-K (Ac)-Pen-AEF-2Nal-THP-K (Ac)-N-3Pya-Sar-CONH2,
MeCO-r-Pen-N-T-7MeW-K (Ac)-Pen-AEF-2Nal-THP-E-N-3Pya-Sar-K (PEG2PEG2gEC16OH)-CONH2,
MeCO-r-Pen-N-T-7MeW-K (Ac)-Pen-AEF-2Nal-THP-E-N-3Pya-K (PEG2PEG2gEC18OH)-CONH2,
MeCO-Pen-N-T-7MeW-K (Ac)-Pen-AEF-2Nal-THP-K (PEG2PEG2gEC16OH)-N-3Pya-Sar-CONH2,
MeCO-Pen-K (PEG2PEG2gEC18OH)-T-7MeW-K (Ac)-Pen-AEF-2Nal-THP-E-N-3Pya-Sar-CONH2,
MeCO-r-Pen-K (PEG2PEG2gEC16OH)-T-7MeW-K (Ac)-Pen-AEF-2Nal-THP-E-N -3Pya-Sar-CONH2,
MeCO-r-Pen-N-T-7MeW-K (Ac)-Pen-AEF-2Nal-THP-E-N-3Pya-NMeK (PEG2PEG2gEC16OH)-CONH2,
MeCO-r-Pen-N-T-7MeW-K (Ac)-Pen-AEF-2Nal-THP-E-N-3Pya-K (PEG2PEG2gEC16OH)-CONH2,
PEG2PEG2gEC16OH-r-Pen-N-T-7MeW-K (Ac)-Pen-AEF-2Nal-THP-K (Ac)-N-3Pya-Sar-CONH2,
MeCO-k (PEG2PEG2gEC16OH)-Pen-N-T-7MeW-K (Ac)-Pen-AEF-2Nal-THP-K (Ac)-N -3Pya-Sar-CONH2,
MeCO-r-Pen-N-T-7MeW-K (Ac)-Pen-AEF (PEG2PEG2gEC16OH)-2Nal-THP-K (Ac)-N -3Pya-Sar-CONH2,
MeCO-r-Pen-N-T-7MeW-K (Ac)-Pen-AEF-2Nal-THP-K (PEG2PEG2gEC18OH)-N -3Pya-Sar-CONH2,
MeCO-r-Pen-N-T-7MeW-K (Ac)-Pen-AEF-2Nal-THP-K (PEG2PEG2gEC16OH)-N -3Pya-Sar-CONH2,
MeCO-Pen-N-T-7MeW-K (Ac)-Pen-AEF-2Nal-THP-K (gEC16OH)-N-3Pya-Sar-CONH2,
MeCO-Pen-N-T-7MeW-K (Ac)-Pen-AEF-2Nal-THP-K (gEC18OH)-N-3Pya-Sar-CONH2,
MeCO-Pen-N-T-7MeW-K (Ac)-Pen-AEF-2Nal-THP-K (gEC18)-N-3Pya-Sar-CONH2, MeCO-Pen-N-T-7MeW-K (Ac)-Pen-AEF-2Nal-THP-K (PEG6gEC16OH)-N-3Pya-Sar-CONH2,
MeCO-Pen-N-T-7MeW-K (Ac)-Pen-AEF-2Nal-THP-K (PEG6gEC18OH)-N-3Pya-Sar-CONH2,
MeCO-Pen-N-T-7MeW-K (Ac)-Pen-AEF-2Nal-THP-K (PEG2gEC16OH)-N-3Pya-Sar-CONH2,
MeCO-Pen-N-T-7MeW-K (Ac)-Pen-AEF-2Nal-THP-K (PEG2gEC18OH)-N-3Pya-Sar-CONH2,
MeCO-Pen-N-T-7MeW-K (Ac)-Pen-AEF-2Nal-THP-K (PEG2PEG2gEC20OH)-N-3Pya-Sar-CONH2,
HOC16gEPEG2PEG2-r-Pen-N-T-7MeW-K (Ac)-Pen-AEF-2Nal-THP-K (PEG2PEG2gEC16OH)-N-3Pya-Sar-CONH2,
MeCO-Pen-N-T-7MeW-K (Ac)-Pen-AEF-2Nal-THP-K (PEG2PEG6gEC16OH)-N-3Pya-Sar-CONH2,
MeCO-Pen-N-T-7MeW-K (Ac)-Pen-AEF-2Nal-THP-K (PEG2PEG6gEC18OH)-N-3Pya-Sar-CONH2,
MeCO-Pen-N-T-7MeW-K (Ac)-Pen-AEF-2Nal-THP-K (PEG2PEG2C16OH)-N-3Pya-Sar-CONH2,
MeCO-Pen-N-T-7MeW-K (Ac)-Pen-AEF-2Nal-THP-K (PEG2PEG2C18OH)-N-3Pya-Sar-CONH2,
MeCO-Pen-N-T-7MeW-K (Ac)-Pen-AEF-2Nal-THP-K (PEG6PEG6gEC16OH)-N-3Pya-Sar-CONH2,
MeCO-Pen-N-T-7MeW-K (Ac)-Pen-AEF-2Nal-THP-K (PEG6PEG6gEC18OH)-N-3Pya-Sar-CONH2,
MeCO-Pen-N-T-7MeW-K (Ac)-Pen-AEF-2Nal-THP-K (PEG24-gEC16OH)-N-3Pya-Sar-CONH2,
MeCO-Pen-N-T-7MeW-K (Ac)-Pen-AEF-2Nal-THP-K (PEG24-gEC18OH)-N-3Pya-Sar-CONH2,
MeCO-Pen-N-T-7MeW-K (Ac)-Pen-AEF-2Nal-THP-K (Ac)-N-3Pya-Sar-PEG2PEG2-eKC18OH-OH,
MeCO-Pen-N-T-7MeW-K (Ac)-Pen-AEF-2Nal-THP-K (PEG24-C18OH)-N-3Pya-Sar-CONH2,
MeCO-k (gEC18OH)-Pen-N-T-7MeW-K (Ac)-Pen-AEF-2Nal-THP-K (Ac)-N-3Pya-Sar-CONH2,
MeCO-k (PEG2gEC18OH)-Pen-N-T-7MeW-K (Ac)-Pen-AEF-2Nal-THP-K (Ac)-N-3Pya-Sar-CONH2,
MeCO-r-Pen-K (PEG2PEG2gEC18OH)-T-7MeW-K (Ac)-Pen-AEF-2Nal-THP-K (Ac)-N -3Pya-Sar-CONH2,
MeCO-r-Pen-K (gEC18OH)-T-7MeW-K (Ac)-Pen-AEF-2Nal-THP-K (Ac)-N-3Pya-Sar-CONH2,
MeCO-r-Pen-K (PEG2gEC18OH)-T-7MeW-K (Ac)-Pen-AEF-2Nal-THP-K (Ac)-N-3Pya-Sar-CONH2,
MeCO-r-Pen-K (PEG2PEG2C18OH)-T-7MeW-K (Ac)-Pen-AEF-2Nal-THP-K (Ac)-N -3Pya-Sar-CONH2,
MeCO-r-Pen-N-T-7MeW-K (Ac)-Pen-AEF-2Nal-THP-K (Ac)-N-3Pya -NMeK (PEG2PEG2gEC18OH)-CONH2,
MeCO-r-Pen-N-T-7MeW-K (Ac)-Pen-AEF-2Nal-THP-K (PEG2PEG2-DgEC18OH)-N -3Pya-Sar-CONH2,
MeCO-r-Pen-N-T-7MeW-K (Ac)-Pen-AEF-2Nal-THP-K (PEG2PEG2-PC18OH)-N -3Pya-Sar-CONH2,
MeCO-r-Pen-N-T-7MeW-K (Ac)-Pen-AEF-2Nal-THP-K (PEG2PEG2gETrxC18OH)-N -3Pya-Sar-CONH2,
MeCO-r-Pen-N-T-7MeW-K (Ac)-Pen-AEF-2Nal-THP-K (PEG2PEG2gETrxC20OH)-N -3Pya-Sar-CONH2,
MeCO-k (PEG6gEC18OH)-Pen-N-T-7MeW-K (Ac)-Pen-AEF-2Nal-THP-K (Ac)-N-3Pya-Sar-CONH2,
MeCO-k (PEG2PEG6gEC18OH)-Pen-N-T-7MeW-K (Ac)-Pen-AEF-2Nal-THP-K (Ac) -N-3Pya-Sar-CONH2,
MeCO-k (PEG2PEG2C18OH)-Pen-N-T-7MeW-K (Ac)-Pen-AEF-2Nal-THP-K (Ac)-N -3Pya-Sar-CONH2,
MeCO-r-Pen-K (PEG6gEC18OH)-T-7MeW-K (Ac)-Pen-AEF-2Nal-THP-K (Ac)-N-3Pya-Sar-CONH2,
MeCO-r-Pen-K (PEG2PEG6gEC18OH)-T-7MeW-K (Ac)-Pen-AEF-2Nal-THP-K (Ac)-N -3Pya-Sar-CONH2,
MeCO-r-Pen-N-T-7MeW-K (Ac)-Pen-AEF-2Nal-THP-K (PEG2PEG2-PPPC18OH)-N -3Pya-Sar-CONH2,
MeCO-k (gEC16)-Pen-N-T-7MeW-K (Ac)-Pen-AEF-2Nal-THP-K (Ac)-N-3Pya-Sar-CONH2,
MeCO-k (gEC18)-Pen-N-T-7MeW-K (Ac)-Pen-AEF-2Nal-THP-K (Ac)-N-3Pya-Sar-CONH2,
MeCO-r-Pen-K (gEC16)-T-7MeW-K (Ac)-Pen-AEF-2Nal-THP-K (Ac)-N-3Pya-Sar-CONH2,
MeCO-r-Pen-K (gEC18)-T-7MeW-K (Ac)-Pen-AEF-2Nal-THP-K (Ac)-N-3Pya-Sar-CONH2,
MeCO-k (PEG2PEG2gETrxC18OH)-Pen-N-T-7MeW-K (Ac)-Pen-AEF-2Nal-THP -K (Ac)-N-3Pya-Sar-CONH2,
MeCO-k (PEG2PEG2gETrxC20OH)-Pen-N-T-7MeW-K (Ac)-Pen-AEF-2Nal-THP -K (Ac)-N-3Pya-Sar-CONH2,
MeCO-Pen-K (PEG2PEG2gETrxC18OH)-T-7MeW-K (Ac)-Pen-AEF-2Nal-THP-K (Ac) -N-3Pya-Sar-CONH2,
MeCO-Pen-K (PEG2PEG2gETrxC20OH)-T-7MeW-K (Ac)-Pen-AEF-2Nal-THP-K (Ac) -N-3Pya-Sar-CONH2,
MeCO-Pen-N-T-7MeW-K (Ac)-Pen-AEF-2Nal-THP-K (gEC16)-N-3Pya-Sar-CONH2,
MeCO-k (PEG2PEG2gEC16OH)-Pen-K (PEG2PEG2gEC16OH)-T-7MeW-K (Ac)-Pen -AEF-2Nal-THP-K (Ac)-N-3Pya-Sar-CONH2,
MeCO-k (PEG2PEG2gEC18OH)-Pen-K (PEG2PEG2gEC18OH)-T-7MeW-K (Ac)-Pen -AEF-2Nal-THP-K (Ac)-N-3Pya-Sar-CONH2,
MeCO-k (PEG2PEG2gEC20OH)-Pen-K (PEG2PEG2gEC20OH)-T-7MeW-K (Ac)-Pen -AEF-2Nal-THP-K (Ac)-N-3Pya-Sar-CONH2,
MeCO-k (gEC16)-Pen-K (gEC16)-T-7MeW-K (Ac)-Pen-AEF-2Nal-THP-K (Ac)-N-3Pya-Sar-CONH2,
MeCO-k (gEC18)-Pen-K (gEC18)-T-7MeW-K (Ac)-Pen-AEF-2Nal-THP-K (Ac)-N-3Pya-Sar-CONH2,
MeCO-k (PEG2PEG2gEC16)-Pen-N-T-7MeW-K (Ac)-Pen-AEF-2Nal-THP-K (Ac)-N -3Pya-Sar-CONH2,
MeCO-k (PEG2PEG2gEC18)-Pen-N-T-7MeW-K (Ac)-Pen-AEF-2Nal-THP-K (Ac)-N -3Pya-Sar-CONH2,
MeCO-Pen-K (PEG2PEG2gEC16)-T-7MeW-K (Ac)-Pen-AEF-2Nal-THP-K (Ac)-N -3Pya-Sar-CONH2,
MeCO-Pen-K (PEG2PEG2gEC18)-T-7MeW-K (Ac)-Pen-AEF-2Nal-THP-K (Ac)-N -3Pya-Sar-CONH2,
MeCO-Pen-K (1PEG21PEG2IsoGluC16Diacid)-T-7MeW-K (Ac)-Pen-AEF-2Nal-THP -K (Ac)-N-3Pya-Sar-CONH2,
MeCO-k (PEG2gEC18OH)-Pen-N-T-7MeW-K (Ac)-Pen-AEF-2Nal-THP-K (Ac)-N-3Pya -N (4Am-Benzyl)-Gly-CONH2,
MeCO-k (gEC18OH)-Pen-N-T-7MeW-K (Ac)-Pen-AEF-2Nal-THP-K (Ac)-N-3Pya -N (4Am-Benzyl)-Gly-CONH2,
MeCO-r-Pen-N-T-7MeW-K (PEG2PEG2gEC18OH)-Pen-AEF-2Nal-THP-K (Ac)-N -3Pya-Sar-CONH2,
MeCO-Pen-N-T-7MeW-K (Ac)-Pen-AEF-2Nal-THP-K (gEC20OH)-N-3Pya-Sar-CONH2,
MeCO-Pen-N-T-7MeW-K (Ac)-Pen-AEF-2Nal-THP-K (PEG2PEG2-TrxgEC18OH)-N -3Pya-Sar-CONH2,
MeCO-Pen-N-T-7MeW-K (Ac)-Pen-AEF-2Nal-THP-K (PEG2PEG2-TrxgETrxC20OH) -N-3Pya-Sar-CONH2,
MeCO-Pen-N-T-7MeW-K (Ac)-Pen-AEF-2Nal-THP-K (PEG2PEG2-TrxgEC20OH)-N -3Pya-Sar-CONH2,
MeCO-k (PEG2PEG2gEC10OH)-Pen-N-T-7MeW-K (Ac)-Pen-AEF-2Nal-THP -K (gEC16)-N-3Pya-Sar-CONH2,
MeCO-k (PEG2PEG2gEC18OH)-Pen-K (gEC16)-T-7MeW-K (Ac)-Pen-AEF-2Nal-THP -K (Ac)-N-3Pya-Sar-CONH2,
MeCO-r-Pen-K (PEG2PEG2PgEC18OH)-T-7MeW-K (Ac)-Pen-AEF-2Nal-THP-K (Ac) -N-3Pya-Sar-CONH2,
MeCO-Pen-N-T-7MeW-K (Ac)-Pen-AEF-2Nal-THP-K (PEG2PEG2gEmXOH)-N-3Pya-Sar-CONH2,
MeCO-k (PEG2PEG2gEC18OH)-Pen-N-T-7MeW-K (Ac)-Pen-AEF-2Nal-THP -K (gEC16)-N-3Pya-Sar-CONH2,
MeCO-r-Pen-K (PEG2PEG2PPPgEC18OH)-T-7MeW-K (Ac)-Pen-AEF-2Nal-THP -K (Ac)-N-3Pya-Sar-CONH2,
MeCO-k (PEG2PEG2PgEC18OH)-Pen-N-T-7MeW-K (Ac)-Pen-AEF-2Nal-THP-K (Ac)-N-3Pya-Sar-CONH2,
MeCO-k (PEG2PEG2PPPgEC18OH)-Pen-N-T-7MeW-K (Ac)-Pen-AEF-2Nal-THP -K (Ac)-N-3Pya-Sar-CONH2,
MeCO-Pen-N-T-7MeW-K (Ac)-Pen-AEF-2Nal-THP-K (PEG2PEG2PgEC18OH)-N -3Pya-Sar-CONH2,
MeCO-Pen-N-T-7MeW-K (Ac)-Pen-AEF-2Nal-THP-K (PEG2PEG2PPPgEC18OH)-N -3Pya-Sar-CONH2,
MeCO-k (PEG2PEG2-TrxgEC18OH)-Pen-N-T-7MeW-K (Ac)-Pen-AEF-2Nal-THP -K (Ac)-N-3Pya-Sar-CONH2,
MeCO-k (PEG2PEG2gEmXOH)-Pen-N-T-7MeW-K (Ac)-Pen-AEF-2Nal-THP-K (Ac)-N -3Pya-Sar-CONH2,
MeCO-Pen-N-T-7MeW-K (Ac)-Pen-AEF-2Nal-THP-K (PEG2PEG2gEpXOH)-N-3Pya-Sar-CONH2,
MeCO-k (PEG2PEG2gEpXOH)-Pen-N-T-7MeW-K (Ac)-Pen-AEF-2Nal-THP -K (PEG2PEG2gEpXOH)-N-3Pya-Sar-CONH2,
MeCO-k (PEG2PEG2gEmXOH)-Pen-N-T-7MeW-K (Ac)-Pen-AEF-2Nal-THP -K (gEC16)-N-3Pya-Sar-CONH2,
MeCO-Pen-N-T-7MeW-K (Ac)-Pen-AEF-2Nal-THP-K (DAP (C16OH) 2)-N-3Pya-Sar-CONH2,
MeCO-k (DAP (C16OH) 2)-Pen-N-T-7MeW-K (Ac)-Pen-AEF-2Nal-THP-K (Ac)-N-3Pya-Sar-CONH2,
MeCO-Pen-N-T-7MeW-K (Ac)-Pen-AEF-2Nal-THP-K (PEG2PEG2gEDAP (C16OH) 2) -N-3Pya-Sar-CONH2,
MeCO-k (PEG2PEG2gEDAP (C16OH) 2)-Pen-N-T-7MeW-K (Ac)-Pen-AEF-2Nal-THP -K (Ac)-N-3Pya-Sar-CONH2,
MeCO-Pen-N-T-7MeW-K (Ac)-Pen-AEF-2Nal-THP-K (PEG2PEG2Sp6gEC18OH)-N -3Pya-Sar-CONH2,
MeCO-Pen-N-T-7MeW-K (Ac)-Pen-AEF-2Nal-THP-K (PEG2Sp6PEG2gEC18OH)-N -3Pya-Sar-CONH2,
MeCO-k (PEG2PEG2gEC16OH)-Pen-N-T-7MeW-K (Ac)-Pen-AEF-2Nal-THP -K (gEC18)-N-3Pya-Sar-CONH2,
MeCO-k (PEG2PEG2gEC18OH)-Pen-N-T-7MeW-K (Ac)-Pen-AEF-2Nal-THP -K (gEC18)-N-3Pya-Sar-CONH2,
MeCO-Pen-K (1PEG21PEG2IsoGluC18Diacid)-T-7MeW-K (Ac)-Pen-AEF-2Nal-THP -K (Ac)-N-3Pya-Sar-CONH2,
MeCO-Pen-K (PEG2PEG2gEC18OH)-T-7MeW-K (Ac)-Pen-AEF-2Nal-THP -K (PEG2PEG2gESp6C18OH)-N-3Pya-Sar-CONH2,
MeCO-Pen-K (PEG2PEG2gEC18OH)-T-7MeW-K (Ac)-Pen-AEF-2Nal-THP -K (Sp6PEG2PEG2gEC18OH)-N-3Pya-Sar-CONH2,
MeCO-k (gEC16)-Pen-N-T-7MeW-K (Ac)-Pen-AEF-2Nal-THP-K (gEC18)-N-3Pya-Sar-CONH2,
MeCO-k (gEC18)-Pen-N-T-7MeW-K (Ac)-Pen-AEF-2Nal-THP-K (gEC18)-N-3Pya-Sar-CONH2,
MeCO-k (PEG2PEG2gEC10OH)-Pen-N-T-7MeW-K (Ac)-Pen-AEF-2Nal-THP -K (gEC18)-N-3Pya-Sar-CONH2,
MeCO-Pen-K (PEG2PEG2gEC18OH)-T-7MeW-K (Ac)-Pen-AEF-2Nal-THP -K (PEG2PEG2gETrxC20OH)-N-3Pya-Sar-CONH2,
MeCO-r-Pen-N-T-7MeW-K (Ac)-Pen-AEF-2Nal-THP-K (Ac)-N-3Pya-Sar-PEG2PEG2-gDabC18OH-OH,
MeCO-k (PEG2PEG2gESp6C18OH)-Pen-N-T-7MeW-K (Ac)-Pen-AEF-2Nal-THP -K (Ac)-N-3Pya-Sar-CONH2,
MeCO-k (PEG2Sp6PEG2gEC18OH)-Pen-N-T-7MeW-K (Ac)-Pen-AEF-2Nal-THP -K (Ac)-N-3Pya-Sar-CONH2,
MeCO-k (Sp6PEG2PEG2gEC18OH)-Pen-N-T-7MeW-K (Ac)-Pen-AEF-2Nal-THP -K (Ac)-N-3Pya-Sar-CONH2,
MeCO-K [PEG2PEG2-gEDAP (pXOH) 2] -Pen-N-T-7MeW-K (Ac)-Pen-AEF-2Nal-THP -K (Ac)-N-3Pya-Sar-CONH2,
MeCO-Pen-N-T-7MeW-K (Ac)-Pen-AEF-2Nal-THP-K [PEG2PEG2-gEDAP-(mXOH) 2]-N-3Pya-Sar-CONH2,
MeCO-k (GolAC16)-Pen-N-T-7MeW-K (Ac)-Pen-AEF-2Nal-THP-K (Ac)-N-3Pya-Sar-CONH2,
MeCO-k (GolAC16OH)-Pen-N-T-7MeW-K (Ac)-Pen-AEF-2Nal-THP-K (Ac)-N-3Pya-Sar-CONH2,
MeCO-k (GolAC18OH)-Pen-N-T-7MeW-K (Ac)-Pen-AEF-2Nal-THP-K (Ac)-N-3Pya-Sar-CONH2,
MeCO-k (PEG2PEG2 GolAC18OH)-Pen-N-T-7MeW-K (Ac)-Pen-AEF-2Nal-THP -K (Ac)-N-3Pya-Sar-CONH2,
MeCO-k (PEG2PEG2gE C18OH (c)-Pen-N-T-7MeW-K (Ac)-Pen-AEF-2Nal-THP -K (Ac)-N-3Pya-Sar-CONH2,
MeCO-k (PEG2PEG2-C18GolB)-Pen-N-T-7MeW-K (Ac)-Pen-AEF-2Nal-THP-K (Ac)-N -3Pya-Sar-CONH2,
MeCO-k (PEG2PEG2gE (C) C18OH)-Pen-N-T-7MeW-K (Ac)-Pen-AEF-2Nal-THP -K (Ac)-N-3Pya-Sar-CONH2,
MeCO-Pen-L-T-7MeW-K (PEG12_OMe)-Pen-F (4-OMe)-2Nal-aMeLeu-L-N-3Pya-Sar-CONH2,
MeCO-Pen-N-T-7MeW-K (PEG12_OMe)-Pen-F (4-OMe)-2Nal-aMeLeu-L-N-3Pya-Sar-CONH2,
MeCO-K (PEG12_OMe)-Pen-L-T-7MeW-K (Ac)-Pen-F (4-OMe)-2Nal-aMeLeu-L-N-3Pya-Sar-CONH2,
MeCO-K (PEG12_OMe)-Pen-N-T-7MeW-K (Ac)-Pen-F (4-OMe)-2Nal-aMeLeu-L-N-3Pya-Sar-CONH2,
MeCO-K (PEG12_OMe)-Pen-N-T-7MeW-K (Ac)-Pen-F-2Nal-aMeLeu-L-N-3Pya-Sar-CONH2,
MeCO-K (PEG12_OMe)-Pen-L-T-7MeW-K (Ac)-Pen-F-2Nal-aMeLeu-L-N-3Pya-Sar-CONH2,
PEG12_OMe-Pen-N-T-7MeW-K (Ac)-Pen-F-2Nal-aMeLeu-L-N-3Pya-Sar-CONH2,
MeCO-Pen-N-T-7MeW-K (PEG12_OMe)-Pen-F-2Nal-aMeLeu-L-N-3Pya-Sar-CONH2,
PEG12 OMe-Pen-L-T-7MeW-K (Ac)-Pen-F-2Nal-aMeLeu-L-N-3Pya-Sar-CONH2,
MeCO-Pen-L-T-7MeW-K (PEG12_OMe)-Pen-F-2Nal-aMeLeu-L-N-3Pya-Sar-CONH2,
PEG4 OMe-Pen-N-T-7MeW-K (Ac)-Pen-F-2Nal-aMeLeu-L-N-3Pya-Sar-CONH2,
MeCO-Pen-N-T-7MeW-K (PEG4)-Pen-F-2Nal-aMeLeu-L-N-3Pya-Sar-CONH2,
PEG4 OMe-Pen-L-T-7MeW-K (Ac)-Pen-F-2Nal-aMeLeu-L-N-3Pya-Sar-CONH2,
MeCO-Pen-L-T-7MeW-K (PEG4)-Pen-F-2Nal-aMeLeu-L-N-3Pya-Sar-CONH2,
MeCO-K (PEG4)-Pen-N-T-7MeW-K (Ac)-Pen-F-2Nal-aMeLeu-L-N-3Pya-Sar-CONH2,
MeCO-K (PEG4)-Pen-L-T-7MeW-K (Ac)-Pen-F-2Nal-aMeLeu-L-N-3Pya-Sar-CONH2,
MeCO-Pen-N-T-7MeW-K (Ac)-Pen-F (4-OMe)-2Nal-aMeK (PEG12_IsoGlu_Palm)-E-L-3Pya-Sar-CONH2,
MeCO-Pen-N-T-7MeW-K (Ac)-Pen-F (4-OMe)-2Nal-aMeK (PEG12_IsoGlu_Palm)-K (Ac)-L-3Pya-Sar-CONH2,
MeCO-Pen-N-T-7MeW-K (Ac)-Pen-2Nal-2Nal-aMeK (PEG12_IsoGlu_Palm)-E-L-3Pya-Sar-CONH2,
MeCO-Pen-N-T-7MeW-K (Ac)-Pen-2Nal-2Nal-aMeK (PEG12_IsoGlu_Palm)-K (Ac)-L-3Pya-Sar-CONH2,
MeCO-Pen-N-T-7MeW-K (Ac)-Pen-F-2Nal-aMeK (PEG12_IsoGlu_Palm)-L-L-3Pya-Sar-CONH2,
MeCO-Pen-N-T-7MeW-K (Ac)-Pen-F-2Nal-aMeK (PEG12_IsoGlu_Palm)-E-L-3Pya-Sar-CONH2,
MeCO-Pen-A-T-7MeW-K (Ac)-Pen-F (4-OMe)-2Nal-aMeK (PEG12_IsoGlu_Palm)-A-A-3Pya-Sar-CONH2,
MeCO-Pen-A-T-7MeW-K (Ac)-Pen-F (4-OMe)-2Nal-aMeK (PEG12_IsoGlu_C18_Diacid)-A-A-3Pya-Sar-CONH2,
MeCO-Pen-N-T-7MeW-K (Ac)-Pen-F (4-OMe)-2Nal-aMeK (PEG12_IsoGlu_Palm)-A-A-3Pya-Sar-CONH2,
MeCO-Pen-A-T-7MeW-K (Ac)-Pen-F (4-OMe)-2Nal-aMeK (PEG12_IsoGlu_Palm)-K (Ac)-N-3Pya-Sar-CONH2,
MeCO-Pen-A-T-7MeW-K (Ac)-Pen-F (4-OMe)-2Nal-aMeK (PEG12_IsoGlu_C18_Diacid)-K (Ac)-N-3Pya-Sar-CONH2,
MeCO-Pen-N-T-7MeW-K (Ac)-Pen-F (4-(2-(1PEG2_1PEG2_IsoGlu_Palm) aminoethoxy))-2Nal-THP-K (Ac)-L-3Pya-Sar-CONH2,
MeCO-Pen-N-T-7MeW-K (Ac)-Pen-F (4-(2-(1PEG21PEG2IsoGluC18Diacid) aminoethoxy))-2Nal-THP-K (Ac)-L-3Pya-Sar-CONH2,
MeCO-Pen-N-T-7MeW-K (Ac)-Pen-F (4-(2-(PEG4_PEG4_IsoGlu_Palm) aminoethoxy))-2Nal-THP-K (Ac)-L-3Pya-Sar-CONH2,
MeCO-Pen-N-T-7MeW-K (Ac)-Pen-F (4-(2-(PEG12_IsoGlu_Palm) aminoethoxy))-2Nal-THP-K (Ac)-L-3Pya-Sar-CONH2,
MeCO-Pen-N-T-7MeW-K (Ac)-Pen-F (4-OMe)-2Nal-K (PEG12_IsoGlu_Palm)-K (Ac)-N-3Pya-Sar-CONH2,
MeCO-Pen-N-T-7MeW-K (Ac)-Pen-F-2Nal-Spiral_Pip_PEG12_IsoGlu_Palm-K (Ac)-N-3Pya-Sar-CONH2,
MeCO-Pen-N-T-7MeW-K (Ac)-Pen-F-2Nal-aMeK (PEG12_IsoGlu_Palm)-K (Ac)-L-3Pya-Sar-CONH2,
MeCO-Pen-N-T-7MeW-K (Ac)-Pen-F (4-OMe)-2Nal-aMeK (PEG12_IsoGlu_C18_Diacid)-A-A-3Pya-Sar-CONH2,
MeCO-Pen-L-T-7MeW-K (Ac)-Pen-F (4-OMe)-2Nal-THP-K (Ac)-L-3Pya-k (PEG12_C18_Diacid)-CONH2,
MeCO-Pen-L-T-7MeW-K (Ac)-Pen-F (4-OMe)-2Nal-THP-K (Ac)-L-3Pya-k (PEG12_IsoGlu_Palm)-CONH2,
MeCO-Pen-L-T-7MeW-K (Ac)-Pen-F (4-OMe)-2Nal-THP-K (Ac)-L-3Pya-k (PEG12_IsoGlu_C18_Diacid)-CONH2,
MeCO-Pen-L-T-7MeW-K (Ac)-Pen-F (4-OMe)-2Nal-THP-K (Ac)-K (PEG12_C18_Diacid-3Pya-Sar-CONH2,
MeCO-Pen-A-T-7MeW-K (Ac)-Pen-F (4-OMe)-2Nal-aMeK (PEG12_IsoGlu_Palm)-K (Ac)-A-3Pya-Sar-CONH2,
MeCO-Pen-A-T-7MeW-K (Ac)-Pen-F (4-OMe)-2Nal-aMeK (PEG12_IsoGlu_C18_Diacid)-K (Ac)-A-3Pya-Sar-CONH2,
MeCO-Pen-L-T-7MeW-K (Ac)-Pen-F (4-OMe)-2Nal-THP-K (PEG12_C18 Diacid-L-3Pya-Sar-CONH2,
MeCO-Pen-L-T-7MeW-K (Ac)-Pen-F (4-OMe)-2Nal-THP-K (PEG12_IsoGlu_Palm)-L-3Pya-Sar-CONH2,
MeCO-Pen-L-T-7MeW-K (Ac)-Pen-F (4-OMe)-2Nal-THP-K (PEG12_IsoGlu_C18_Diacid-L-3Pya-Sar-CONH2,
MeCO-Pen-L-K (PEG12_C18_Diacid-7MeW-K (Ac)-Pen-F (4-OMe)-2Nal-THP-K (Ac)-L -3Pya-Sar-CONH2,
MeCO-Pen-L-K (PEG12_IsoGlu_Palm)-7MeW-K (Ac)-Pen-F (4-OMe)-2Nal-THP-K (Ac)-L-3Pya-Sar-CONH2,
MeCO-Pen-L-K (PEG12_IsoGlu_C18_Diacid-7MeW-K (Ac)-Pen-F (4-OMe)-2Nal-THP-K (Ac)-L-3Pya-Sar-CONH2,
MeCO-Pen-N-T-7MeW-K (Ac)-Pen-F (4-OMe)-2Nal-aMeK (PEG12_IsoGlu_Palm)-K (Ac)-A-3Pya-Sar-CONH2,
MeCO-Pen-K (PEG12_IsoGlu_Palm)-T-7MeW-K (Ac)-Pen-F (4-OMe)-2Nal-THP-K (Ac)-L-3Pya-Sar-CONH2,
MeCO-Pen-K (PEG12_IsoGlu_C18_Diacid-T-7MeW-K (Ac)-Pen-F (4-OMe)-2Nal-THP-K (Ac)-L-3Pya-Sar-CONH2,
-Pen (PEG4_Ahx_C18_Diacid)-N-T-7MeW-K (Ac)-Pen-F (4-OMe)-2Nal-THP-E-L-3Pya-Sar-CONH2,
-Pen (PEG4_IsoGlu_C18_Diacid)-N-T-7MeW-K (Ac)-Pen-F (4-OMe)-2Nal-THP-E-L-3Pya-Sar-CONH2,
MeCO-k (PEG12_IsoGlu_Palm)-Pen-L-T-7MeW-K (Ac)-Pen-F (4-OMe)-2Nal-THP-L-L-3Pya-Sar-CONH2,
MeCO-k (PEG12_IsoGlu_C18_Diacid)-Pen-L-T-7MeW-K (Ac)-Pen-F (4-OMe)-2Nal-THP -L-L-3Pya-Sar-CONH2,
MeCO-k (PEG12_C18_Diacid)-Pen-L-T-7MeW-K (Ac)-Pen-F (4-OMe)-2Nal-THP-K (Ac)-L-3Pya-Sar-CONH2,
MeCO-k (Peg4_C18_Diacid)-Pen-L-T-7MeW-K (Ac)-Pen-F (4-OMe)-2Nal-THP-K (Ac)-L -3Pya-Sar-CONH2,
MeCO-k (IsoGlu_C18_Diacid)-Pen-L-T-7MeW-K (Ac)-Pen-F (4-OMe)-2Nal-THP-K (Ac)-L-3Pya-Sar-CONH2,
MeCO-k (Peg4_IsoGlu_C18_Diacid)-Pen-L-T-7MeW-K (Ac)-Pen-F (4-OMe)-2Nal-THP-K (Ac)-L-3Pya-Sar-CONH2,
MeCO-k (PEG12_IsoGlu_Palm)-Pen-L-T-7MeW-K (Ac)-Pen-F (4-OMe)-2Nal-THP-K (Ac)-L-3Pya-Sar-CONH2,
MeCO-k (PEG12_IsoGlu_C18_Diacid)-Pen-L-T-7MeW-K (Ac)-Pen-F (4-OMe)-2Nal-THP -K (Ac)-L-3Pya-Sar-CONH2,
MeCO-Pen-L-T-7MeW-K (PEG12_C18_Diacid-Pen-F (4-OMe)-2Nal-THP-L-L-3Pya-Sar-CONH2,
MeCO-Pen-L-T-7MeW-K (PEG4_C18_Diacid)-Pen-F (4-OMe)-2Nal-THP-L-L-3Pya-Sar-CONH2,
MeCO-Pen-L-T-7MeW-K (IsoGlu_C18_Diacid-Pen-F (4-OMe)-2Nal-THP-L-L-3Pya-Sar-CONH2,
MeCO-Pen-L-T-7MeW-K (IsoGlu_Palm)-Pen-F (4-OMe)-2Nal-THP-L-L-3Pya-Sar-CONH2,
MeCO-Pen-L-T-7MeW-K (PEG4_IsoGlu_Palm)-Pen-F (4-OMe)-2Nal-THP-L-L-3Pya-Sar-CONH2,
MeCO-Pen-L-T-7MeW-K (PEG4_IsoGlu_C18_Diacid-Pen-F (4-OMe)-2Nal-THP-L-L-3Pya-Sar-CONH2,
MeCO-Pen-L-T-7MeW-K (PEG12_IsoGlu_Palm)-Pen-F (4-OMe)-2Nal-THP-L-L-3Pya-Sar-CONH2,
MeCO-Pen-L-T-7MeW-K (PEG12_IsoGlu_C18_Diacid-Pen-F (4-OMe)-2Nal-THP-L-L-3Pya-Sar-CONH2,
MeCO-Pen-L-T-7MeW-K (PEG12_C18_Diacid-Pen-F (4-OMe)-2Nal-THP-K (Ac)-L-3Pya-Sar-CONH2,
MeCO-Pen-L-T-7MeW-K (PEG4_C18_Diacid)-Pen-F (4-OMe)-2Nal-THP-K (Ac)-L-3Pya-Sar-CONH2,
MeCO-Pen-L-T-7MeW-K (IsoGlu_C18_Diacid-Pen-F (4-OMe)-2Nal-THP-K (Ac)-L-3Pya-Sar-CONH2,
MeCO-Pen-L-T-7MeW-K (IsoGlu_Palm)-Pen-F (4-OMe)-2Nal-THP-K (Ac)-L-3Pya-Sar-CONH2,
MeCO-Pen-L-T-7MeW-K (PEG4_IsoGlu_Palm)-Pen-F (4-OMe)-2Nal-THP-K (Ac)-L-3Pya-Sar-CONH2,
MeCO-Pen-L-T-7MeW-K (PEG4_IsoGlu_C18_Diacid-Pen-F (4-OMe)-2Nal-THP-K (Ac) -L-3Pya-Sar-CONH2,
MeCO-Pen-L-T-7MeW-K (PEG12_IsoGlu_Palm)-Pen-F (4-OMe)-2Nal-THP-K (Ac)-L-3Pya-Sar-CONH2,
MeCO-Pen-L-T-7MeW-K (PEG12_IsoGlu_C18_Diacid-Pen-F (4-OMe)-2Nal-THP-K (Ac)-L-3Pya-Sar-CONH2,
MeCO-Pen-N-T-7MeW-K (Ac)-Pen-F (4-OMe)-2Nal-aMeK (PEG12_IsoGlu_C18_Diacid)-K (Ac)-A-3Pya-Sar-CONH2,
MeCO-Pen-N-T-7MeW-K (Ac)-Pen-F (4-(2-(PEG4_PEG4_IsoGlu_C18_Diacid) aminoethoxy)) -2Nal-THP-K (Ac)-L-3Pya-Sar-CONH2,
MeCO-Pen-N-T-7MeW-K (Ac)-Pen-F (4-(2-(PEG12_IsoGlu_C18_Diacid) aminoethoxy)) -2Nal-THP-K (Ac)-L-3Pya-Sar-CONH2,
MeCO-Pen-N-T-7MeW-K (Ac)-Pen-F (4-OMe)-2Nal-K (PEG12_IsoGlu_C18_Diacid-K (Ac)-N-3Pya-Sar-CONH2,
MeCO-Pen-L-T-7MeW-K (Ac)-Pen-F (4-OMe)-2Nal-THP-K (Ac)-K (PEG12_IsoGlu_C18_Diacid-3Pya-Sar-CONH2,
MeCO-Pen-K (PEG12_C18_Diacid-T-7MeW-K (Ac)-Pen-F (4-OMe)-2Nal-THP-K (Ac)-L -3Pya-Sar-CONH2,
PEG4_Decyl-Pen-N-T-7MeW-K (Ac)-Pen-F (4-OMe)-2Nal-THP-L-N-3Pya-Sar-CONH2,
PEG4_Lauryl-Pen-N-T-7MeW-K (Ac)-Pen-F (4-OMe)-2Nal-THP-L-N-3Pya-Sar-CONH2,
MeCO-Pen-N-T-7MeW-K (Ac)-Pen-AEF-2Nal-THP-E-N-3Pya-Sar-k (PEG12_IsoGlu_C18_Diacid)-CONH2,
PEG4_Capryl-Pen-N-T-7MeW-K (Ac)-Pen-F (4-OMe)-2Nal-THP-L-N-3Pya-Sar-CONH2,
PEG4_Hexyl-Pen-N-T-7MeW-K (Ac)-Pen-F (4-OMe)-2Nal-THP-L-N-3Pya-Sar-CONH2,
PEG2 Palm-Pen-N-T-7MeW-K (Ac)-Pen-F (4-OMe)-2Nal-THP-L-N-3Pya-Sar-CONH2,
PEG2_Myristyl-Pen-N-T-7MeW-K (Ac)-Pen-F (4-OMe)-2Nal-THP-L-N-3Pya-Sar-CONH2,
PEG2_Lauryl-Pen-N-T-7MeW-K (Ac)-Pen-F (4-OMe)-2Nal-THP-L-N-3Pya-Sar-CONH2,
Hexyl-Pen-N-T-7MeW-K (Ac)-Pen-F (4-OMe)-2Nal-THP-L-N-3Pya-Sar-CONH2,
Decyl-Pen-N-T-7MeW-K (Ac)-Pen-F (4-OMe)-2Nal-THP-L-N-3Pya-Sar-CONH2,
PEG2_Decyl-Pen-N-T-7MeW-K (Ac)-Pen-F (4-OMe)-2Nal-THP-L-N-3Pya-Sar-CONH2,
PEG2_Capryl-Pen-N-T-7MeW-K (Ac)-Pen-F (4-OMe)-2Nal-THP-L-N-3Pya-Sar-CONH2,
Oct-Pen-N-T-7MeW-K (Ac)-Pen-F (4-OMe)-2Nal-THP-L-N-3Pya-Sar-CONH2,
MeCO-Pen-N-T-7MeW-K (Ac)-Pen-AEF-2Nal-THP-E-N-3Pya-Sar-k (Peg4_IsoGlu_Palm)-CONH2,
MeCO-Pen-N-T-7MeW-K (Ac)-Pen-AEF-2Nal-THP-E-N-3Pya-Sar-k (IsoGlu_Palm)- CONH2,
MeCO-Pen-N-T-7MeW-K (Ac)-Pen-AEF-2Nal-THP-E-N-3Pya-Sar-K (PEG12_C18_Diacid)-CONH2,
MeCO-Pen-N-T-7MeW-K (Ac)-Pen-F (4-CONH2,)-2Nal-aMeK (Peg4_IsoGlu_C18_Diacid)-K (Ac)-N-3Pya-Sar-CONH2,
MeCO-Pen-N-T-7MeW-K (Ac)-Pen-F (4-CONH2)-2Nal-aMeK (PEG12_C18_Diacid)-K (Ac)-N-3Pya-Sar-CONH2,
MeCO-Pen-L-T-7MeW-K (Ac)-Pen-F (4-OMe)-2Nal-THP-K (Ac)-K (PEG12_IsoGlu_Palm)-3Pya-Sar-CONH2,
PEG2 Palm-Pen-N-T-7MeW-K (Ac)-Pen-AEF-2Nal-THP-E-N-3Pya-Sar-CONH2,
PEG2_Lauryl-Pen-N-T-7MeW-K (Ac)-Pen-AEF-2Nal-THP-E-N-3Pya-Sar-CONH2,
MeCO-Pen-N-T-7MeW-K (Ac)-Pen-AEF-2Nal-THP-E-N-3Pya-Sar-K (Peg4_IsoGlu_C18_Diacid)-CONH2,
MeCO-Pen-N-T-7MeW-K (Ac)-Pen-AEF-2Nal-THP-E-N-3Pya-Sar-K (PEG12_IsoGlu_Palm)-CONH2,
MeCO-Pen-N-T-7MeW-K (Ac)-Pen-AEF-2Nal-THP-E-N-3Pya-Sar-K (Peg4_C18_Diacid)-CONH2,
MeCO-Pen-N-T-7MeW-K (Ac)-Pen-AEF-2Nal-THP-E-N-3Pya-Sar-K (IsoGlu_C18_Diacid)-CONH2,
PEG4 Palm-Pen-N-T-7MeW-K (Ac)-Pen-AEF-2Nal-THP-E-N-3Pya-Sar-CONH2,
Palm-Pen-N-T-7MeW-K (Ac)-Pen-AEF-2Nal-THP-E-N-3Pya-Sar-CONH2,
Lauryl-Pen-N-T-7MeW-K (Ac)-Pen-AEF-2Nal-THP-E-N-3Pya-Sar-CONH2,
Oct-Pen-N-T-7MeW-K (Ac)-Pen-AEF-2Nal-THP-E-N-3Pya-Sar-CONH2,
PEG4_Lauryl-Pen-N-T-7MeW-K (Ac)-Pen-AEF-2Nal-THP-E-N-3Pya-Sar-CONH2,
PEG4_Capryl-Pen-N-T-7MeW-K (Ac)-Pen-AEF-2Nal-THP-E-N-3Pya-Sar-CONH2,
PEG4_Hexyl-Pen-N-T-7MeW-K (Ac)-Pen-AEF-2Nal-THP-E-N-3Pya-Sar-CONH2,
MeCO-Pen-N-T-7MeW-K (Ac)-Pen-F (4-CONH2)-2Nal-aMeK (PEG12_IsoGlu_C18_Diacid)-K (Ac)-N-3Pya-Sar-CONH2,
MeCO-Pen-N-T-7MeW-K (Ac)-Pen-F (4-CONH2)-2Nal-aMeK (PEG12_IsoGlu_Palm)- K (Ac)-N-3Pya-Sar-CONH2,
MeCO-Pen-N-T-7MeW-K (Ac)-Pen-F (4-CONH2)-2Nal-aMeK (Peg4_IsoGlu_Palm)-K (Ac)-N-3Pya-Sar-CONH2,
MeCO-Pen-N-T-7MeW-K (Ac)-Pen-F (4-CONH2)-2Nal-aMeK (IsoGlu_Palm)-K (Ac)-N-3Pya-Sar-CONH2,
MeCO-Pen-N-T-7MeW-K (Ac)-Pen-F (4-CONH2)-2Nal-aMeK (IsoGlu_C18_Diacid)-K (Ac)-N-3Pya-Sar-CONH2,
MeCO-Pen-N-T-7MeW-K (Ac)-Pen-F (4-CONH2)-2Nal-aMeK (Peg4_C18_Diacid)-K (Ac)-N-3Pya-Sar-CONH2,
1PEG2 1PEG2_IsoGlu_C16_DiMeCOid-r-Pen-N-T-7MeW-K (Ac)-Pen-AEF-2Nal-THP-K (Ac)-N-3Pya-Sar-CONH2,
1PEG2 1PEG2 IsoGlu_C18_DiMeCOid-r-Pen-N-T-7MeW-K (Ac)-Pen-AEF-2Nal-THP-K (Ac)-N-3Pya-Sar-CONH2,
MeCO-r-Pen-N-T-7MeW-K (Ac)-Pen-AEF-2Nal-THP-E-N-3Pya-K (1PEG21PEG2IsoGluC16Diacid)-CONH2,
MeCO-r-Pen-K (1PEG21PEG2IsoGluC16Diacid)-T-7MeW-K (Ac)-Pen-AEF-2Nal-THP-E -N-3Pya-Sar-CONH2,
MeCO-r-Pen-K (1PEG21PEG2IsoGluC18Diacid)-T-7MeW-K (Ac)-Pen-AEF-2Nal-THP-E -N-3Pya-Sar-CONH2,
MeCO-Pen-N-T-7MeW-K (Ac)-Pen-AEF-2Nal-THP-K (1PEG21PEG2IsoGluC16Diacid)-N-3Pya-Sar-CONH2,
MeCO-Pen-N-T-7MeW-K (Ac)-Pen-AEF-2Nal-THP-K (1PEG21PEG2IsoGluC18Diacid)-N-3Pya-Sar-CONH2,
MeCO-r-Pen-N-T-7MeW-K (Ac)-Pen-AEF-2Nal-THP-E-N-3Pya-K (1PEG21PEG2IsoGluC18Diacid)-CONH2,
MeCO-r-Pen-N-T-7MeW-K (Ac)-Pen-AEF-2Nal-THP-E-N-3Pya-Sar-K (1PEG21PEG2IsoGluC16Diacid)-CONH2,
MeCO-r-Pen-N-T-7MeW-K (Ac)-Pen-AEF-2Nal-THP-E-N-3Pya-Sar-K (1PEG21PEG2IsoGluC18Diacid)-CONH2,
1PEG2 1PEG2 IsoGlu C18-r-Pen-N-T-7MeW-K (Ac)-Pen-AEF-2Nal-Acvc-E-N-THP-CONH2,
1PEG2 1PEG2 IsoGlu_C18_DiMeCOid-r-Pen-N-T-7MeW-K (Ac)-Pen-AEF-2Nal-Acvc -E-N-THP-CONH2,
MeCO-r-Pen-N-T-7MeW-K (Ac)-Pen-AEF-2Nal-Acvc-E-N-THP-K (1PEG21PEG2IsoGluC18Diacid)-CONH2,
MeCO-k (1PEG21PEG2IsoGluC18Diacid)-Pen-N-T-7MeW-K (Ac)-Pen-AEF-2Nal-Acvc -E-N-THP-CONH2,
MeCO-k (1PEG21PEG2IsoGluC16Diacid)-Pen-N-T-7MeW-K (Ac)-Pen-AEF-2Nal-Acvc -E-N-THP-CONH2,
MeCO-r-Pen-N-T-7MeW-K (Ac)-Pen-AEF-2Nal-Acvc-E-N-THP-K (1PEG2_1PEG2_IsoGlu_C18)-CONH2,
MeCO-Pen-N-T-7MeW-K (Ac)-Pen-AEF-3Quin-THP-E-N-H-Sar-CONH2,
MeCO-Pen-N-T-7MeW-K (Ac)-Pen-F (4-OMe)-2Nal-aMeK (PEG12_IsoGlu_C18_Diacid)-K (Ac)-N-3Pya-Sar-CONH2,
MeCO-Pen-N-T-7MeW-K (Ac)-Pen-F (4-OMe)-2Nal-aMeK (PEG12_IsoGlu_Palm)-K (Ac)-N-3Pya-Sar-CONH2,
PEG12 OMe-Pen-L-T-7MeW-K (Ac)-Pen-F-2Nal-aMeLeu-L-N-NH (2-(pyridin-3-yl) ethyl),
MeCO-Pen-L-T-7MeW-K (PEG12_OMe)-Pen-F-2Nal-aMeLeu-L-N-NH (2-(pyridin-3-yl) ethyl),
PEG12 OMe-Pen-L-T-7MeW-K (Ac)-Pen-F (4-OMe)-2Nal-aMeLeu-L-N-NH (2-(pyridin-3-yl) ethyl),
MeCO-Pen-L-T-7MeW-K (PEG12_OMe)-Pen-F (4-OMe)-2Nal-aMeLeu-L-N-NH (2-(pyridin-3-yl) ethyl),
PEG12 OMe-Pen-aMeAsn-T-7MeW-K (Ac)-Pen-F-2Nal-aMeLeu-L-N-NH (2-(pyridin-3-yl) ethyl),
MeCO-Pen-aMeAsn-T-7MeW-K (PEG12_OMe)-Pen-F-2Nal-aMeLeu-L-N-NH (2-(pyridin-3-yl) ethyl),
PEG12 OMe-Pen-aMeAsn-T-7MeW-K (Ac)-Pen-F (4-OMe)-2Nal-aMeLeu-L-N-NH (2-(pyridin-3-yl) ethyl),
MeCO-Pen-aMeAsn-T-7MeW-K (PEG12_OMe)-Pen-F (4-OMe)-2Nal-aMeLeu-L-N-NH (2-(pyridin-3-yl) ethyl),
PentCO-r-Pen-Q-T-W-Q-Pen-AEF-2Nal-THP-E-N-bAla-CONH2,
MeCO-r-Pen-Q-T-W-Q-Pen-AEF-2Nal-THP-K (COPent)-N-bAla-CONH2,
PentCO-r-Pen-Q-T-W-Q-Pen-AEF-2Nal-THP-E-N-THP-CONH2,
MeCO-r-Pen-Q-T-W-Q-Pen-AEF-2Nal-THP-K (COPent)-N-F-CONH2,
PentCO-r-Pen-N-T-7MeW-K (Ac)-Pen-AEF-2Nal-THP-E-N-aMePhe-CONH2,
MeCO-r-Pen-N-T-7MeW-K (Ac)-Pen-AEF-2Nal-Acpx-E-N-THP-K (PEG2PEG2gEC18)-CONH2,
MeCO-k (PEG2PEG2gEC18)-Pen-N-T-7MeW-K (Ac)-Pen-AEF-2Nal-Acpx-E-N-THP-CONH2,
MeCO-k (PEG2PEG2gEC18OH)-Pen-N-T-7MeW-K (Ac)-Pen-AEF-2Nal-Acpx-E-N-THP-CONH2,
MeCO-r-Pen-N-T-7MeW-K (Ac)-Pen-AEF-2Nal-Acpx-E-N-THP-K (PEG2PEG2gEC18OH)-CONH2,
HOC18gEPEG2PEG2-r-Pen-N-T-7MeW-K (Ac)-Pen-AEF-2Nal-Acpx-E-N-THP-CONH2,
C18gEPEG2PEG2CO-r-Pen-N-T-7MeW-K (Ac)-Pen-AEF-2Nal-Acpx-E-N-THP-CONH2,
MeCO-Pen-N-T-7MeW-K (Ac)-Pen-AEF-2Nal-aMeK (PEG2PEG2gEC18OH)-K (Ac)-N-3Pya-Sar-CONH2,
HOC10gEPEG2COPEG2CO-r-Pen-N-T-7MeW-K (Ac)-Pen-AEF-2Nal-THP-K (PEG2PEG2gEC10OH)-N-3Pya-Sar-CONH2,
MeCO-Pen-N-T-7MeW-K (Ac)-Pen-AEF-2Nal-THP-K (PEG2PEG2gEC10OH)-N-3Pya-Sar-CONH2,
MeCO-Pen-N-T-7MeW-K (Ac)-Pen-AEF-2Nal-aMeK (PEG2PEG2gEC16OH)-K (Ac)-N-3Pya-Sar-CONH2,
MeCO-T-7MeW-K (Ac)-S5H-AEF-2Nal-THP-S5H-N-3Pya-Sar-K (PEG2PEG2gEC18OH)-CONH2,
MeCO-T-7MeW-K (Ac)-S5H-AEF-2Nal-THP-S5H-N-3Pya-Sar-K (PEG2PEG2gEC160H)-CONH2,
MeCO-k (PEG2PEG2gEC10OH)-Pen-N-T-7MeW-K (Ac)-Pen-AEF-2Nal-THP-K (Ac)- N-3Pya-Sar-CONH2,
MeCO-Pen-K (PEG2PEG2gEC10OH)-T-7MeW-K (Ac)-Pen-AEF-2Nal-THP-K (Ac)-N-3Pya-Sar-CONH2,
MeCO-Pen-N-T-7MeW-K (Ac)-Pen-AEF-2Nal-THP-K (gEC10OH)-N-3Pya-Sar-CONH2,
MeCO-k (PEG2PEG2gEC18OH)-Pen-N-T-7MeW-K (Ac)-Pen-AEF-2Nal-THP-K (Ac)-N -3Pya-4diFPro-CONH2,
7Ahp (2)-Pen-K (1PEG21PEG2IsoGluC18Diacid)-T-7MeW-K (Ac)-Pen-AEF-2Nal-THP-E (2)-N-3Pya-Sar-CONH2,
GABA (2)-k (PEG2PEG2gEC18OH)-Pen-N-T-7MeW-K (Ac)-Pen-AEF-2Nal-THP-E (2)-N -3Pya-Sar-CONH2,
MeCO-k (PEG2PEG2gEC10OH)-Pen-K (PEG2PEG2gEC10OH)-T-7MeW-K (Ac)-Pen-AEF -2Nal-THP-K (Ac)-N-3Pya-Sar-CONH2,
MeCO-k (PEG2PEG2gEC18OH)-Pen-E (2)-T-7MeW-K (Ac)-Pen-AEF-2Nal-THP-K (Ac)-N-3Pya-Sar-CONH2,
MeCO-k (gEC18OH)-Pen-E (2)-T-7MeW-K (Ac)-Pen-AEF-2Nal-THP-K (Ac)-N-3Pya-Sar-CONH2,
MeCO-k (PEG2PEG2C18OH)-Pen-E (2)-T-7MeW-K (Ac)-Pen-AEF-2Nal-THP-K (Ac)-N-3Pya-Sar-CONH2,
6Ahx (2)-Abu-N-T-W-Q-C-AEF-2Nal-THP-E (2)-N-3Pya-NMeK (PEG2PEG2gEC18OH)-CONH2,
6Ahx (2)-Abu-K (1PEG21PEG2IsoGluC18Diacid)-T-W-Q-C-AEF-2Nal-THP-E (2)-N-3Pya-Sar-CONH2,
5Ava (2)-Abu-K (1PEG21PEG2IsoGluC18Diacid)-T-W-Q-C-AEF-2Nal-THP-E (2)-N-3Pya-Sar-CONH2,
MeCO-k (gEC18)-Pen-E (2)-T-7MeW-K (Ac)-Pen-AEF-2Nal-THP-K (Ac)-N-3Pya-Sar-CONH2,
HOC16gEPEG2PEG2orn (2)-r-Pen-N-T-7MeW-K (Ac)-Pen-AEF-2Nal-THP-E (2)-N-3Pya-Sar-CONH2,
CF3CO-k (PEG2PEG2gEC18OH)-Pen-E (2)-T-7MeW-K (Me) 3-Pen-AEF-2Nal-THP-K (Me) 3-N-3Pya-Sar-CON (Me) 2
MeCO-k (PEG2PEG2gEC12OH (C))-Pen-N-T-7MeW-K (Ac)-Pen-AEF-2Nal-THP-K (Ac)-N-3Pya-Sar-CONH2,
MeCO-k (PEG2PEG2gE (C) C12)-Pen-N-T-7MeW-K (Ac)-Pen-AEF-2Nal-THP-K (Ac)-N-3Pya-Sar-CONH2,
MeCO-Pen-N-T-7MeW-K (Ac)-Pen-4AmF-2Nal-aMeK (PEG12gEC16)-K (Ac)-N-3Pya-Sar-CONH2,
MeCO-Pen-N-T-7MeW-K (Ac)-Pen-40MeF-2Nal-aMeK (PEG12gEC16)-K (Ac)-N-3Pya-Sar-CONH2,
MeCO-r-Pen-N-T-7MeW-K (Ac)-Pen-AEF-2Nal-THP-K (FITCPEG4)-N-3Pya-Sar-K (PEG2PEG2gEC18OH)-CONH2,
5Ava-Abu-N-T-W-K (PEG2PEG2gEC18OH)-C-AEF-2Nal-THP-E-N-3Pya-Sar-CONH2,
5Ava-Abu-N-T-W-Q-C-AEF-2Nal-THP-E-N-3Pya-Sar-K (PEG2PEG2gEC18OH)-CONH2,
AEEP-Pen-N-T-7MeW-K (Ac)-Pen-AEF-2Nal-THP-E-N-3Pya-Sar-K (PEG2PEG2gEC18OH)-CONH2,
HOC18gEPEG2PEG2-r-Pen-E (2)-T-7MeW-K (Ac)-Pen-AEF-2Nal-THP-E-N-3Pya-Sar-CONH2,
MeCO-k (PEG2PEG2gEC18OH)-Pen-E (2)-T-7MeW-K (Ac)-Pen-AEF-2Nal-THP-E-N-3Pya-Sar-CONH2,
MeCO-r-Pen-E (2)-T-7MeW-K (PEG2PEG2gEC18OH)-Pen-AEF-2Nal-THP-E-N-3Pya-Sar-CONH2,
MeCO-r-Pen-E (2)-T-7MeW-K (Ac)-Pen-AEF-2Nal-THP-E-N-3Pya-Sar-K (PEG2PEG2gEC18OH)-CONH2,
AEEP-Pen-N-T-7MeW-K (PEG2PEG2gEC18OH)-Pen-AEF-2Nal-THP-E-N-3Pya-Sar-CONH2,
AEEP (2)-Pen-K (1PEG21PEG2IsoGluC18Diacid)-T-7MeW-K (Ac)-Pen-AEF-2Nal-THP-E (2)-N-3Pya-Sar-CONH2,
MeCO-r-Pen-N-T-7MeW-K (Ac)-Pen-AEF (PEG2PEG2gEC18OH)-2Nal-THP-E-N-3Pya-NMeKdCAR-CONH2,
MeCO-kdCar-Pen-N-T-7MeW-K (PEG2PEG2gEC18OH)-Pen-AEF-2Nal-THP-E-N-3Pya-Sar-CONH2,
MeCO-r-Pen-KdCAR-T-7MeW-K (PEG2PEG2gEC18OH)-Pen-AEF-2Nal-THP-E-N-3Pya-Sar-CONH2,
MeCO-Pen-N-T-7MeW-K (PEG2PEG2gEC18OH)-Pen-AEF-2Nal-THP-k-N-3Pya-Sar-CONH2,
MeCO-r-Pen-K (PEG2PEG2gEC18OH)-T-7MeW-k-Pen-AEF-2Nal-THP-E-N-3Pya-Sar-CONH2,
MeCO-r-Pen-K (PEG2PEG2gEC18OH)-T-7MeW-K (Ac)-Pen-AEF-2Nal-THP-E-N-3Pya-dMeG-CONH2,
succiniccarn-k (PEG2PEG2gEC18OH)-Pen-N-T-7MeW-K (Ac)-Pen-AEF-2Nal-THP-E-N-3Pya-Sar-CONH2,
MeCO-r-Pen-K (PEG2PEG2gEC18OH)-T-7MeW-K (Ac)-Pen-dAEF-2Nal-THP-E-N-3Pya-Sar-CONH2,
MeCO-Pen-N-T-W-K (PEG2PEG2gEC20OH)-Pen-AEF-2Nal-THP-K (Ac)-N-3Pya-Sar-CONH2,
MeCO-Pen-K (PEG2PEG2gEC20OH)-T-W-K (Ac)-Pen-AEF-2Nal-THP-K (Ac)-N-3Pya-Sar-CONH2,
MeCO-K (PEG2PEG2gEC20OH)-Pen-N-T-W-K (Ac)-Pen-AEF-2Nal-THP-K (Ac)-N-3Pya-Sar-CONH2,
MeCO-Pen-N-T-W-K (Ac)-Pen-AEF-2Nal-THP-K (Ac)-N-3Pya-Sar-K (PEG2PEG2gEC20OH)-CONH2,
MeCO-k (SP6)-Pen-N-T-7MeW-K (Ac)-Pen-AEF-2Nal-THP-E-N-3Pya-Sar-K (PEG2PEG2gEC18OH)-CONH2,
MeCO-r-Pen-N-T-7MeW-K (Ac)-Pen-AEF-2Nal-THP-k-N-3Pya-Sar-K (PEG2PEG2gEC20OH)-CONH2,
MeCO-Pen-N-T-7MeW-K (Ac)-Pen-AEF-2Nal-THP-KdCAR-N-3Pya-Sar-K (PEG2PEG2gEC20OH)-CONH2,
MeCO-r-Pen-N-T-7MeW-K (PEG2PEG2gEC20OH)-Pen-AEF-2Nal-THP-E-N-3Pya-Sar-CONH2,
cPEG3MeCOO-r-Pen-N-T-7MeW-K (PEG2PEG2gEC20OH)-Pen-AEF-2Nal-THP-E-N-3Pya-Sar-CONH2,
cPEG3MeCOO-r-Pen-K (PEG2PEG2gEC20OH)-T-7MeW-K (Ac)-Pen-AEF-2Nal-THP-E -N-3Pya-Sar-CONH2,
MeCO-r-Pen-N-T-7MeW-K (Ac)-Pen-AEF-2Nal-THP-E-N-3Pya-Sar-K (PEG2PEG2gEC20OH)-CONH2,
MeCO-r-Abu-N-T-7MeW-K (Ac)-C-AEF-2Nal-THP-E-N-3Pya-Sar-K (PEG2PEG2gEC18OH)-CONH2,
MeCO-C-N-T-7MeW-K (Ac)-aMeC-AEF-2Nal-THP-E-N-3Pya-Sar-K (PEG2PEG2gEC18OH)-CONH2,
MeCO-r-Abu-N-T-7MeW-K (Ac)-aMeC-AEF-2Nal-THP-E-N-3Pya-Sar-K (PEG2PEG2gEC18OH)-CONH2,
MeCO-r-Abu-N-T-7MeW-K (Ac)-Pen-AEF-2Nal-THP-E-N-3Pya-Sar-K (PEG2PEG2gEC18OH)-CONH2,
C12gEPEG2PEG2CO-Pen-N-T-7MeW-K (PEG2PEG2gEC12)-Pen-AEF-2Nal-THP-E-N -3Pya-Sar-CONH2,
C14gEPEG2PEG2CO-Pen-N-T-7MeW-K (PEG2PEG2gEC14)-Pen-AEF-2Nal-THP-E-N -3Pya-Sar-CONH2,
MeCO-Pen-N-T-7MeW-K (PEG2PEG2gEC12)-Pen-AEF-2Nal-THP-K (PEG2PEG2gEC12)-N-3Pya-Sar-CONH2,
MeCO-r-Pen-N15-T-7MeW-K (Ac)-Pen-AEF-2Nal-THP-E-N-3Pya-Sar-K (PEG2PEG2gEC18OH)-CONH2,
MeCO-Pen-N-T-7MeW-K (PEG2PEG2gEC14)-Pen-AEF-2Nal-THP-K (PEG2PEG2gEC14)-N-3Pya-Sar-CONH2,
C14gEPEG2PEG2CO-k (PEG2PEG2gEC14)-Pen-N-T-7MeW-K (Ac)-Pen-AEF-2Nal-THP -E-N-3Pya-Sar-CONH2,
C12gEPEG2PEG2CO-k (PEG2PEG2gEC12)-Pen-N-T-7MeW-K (Ac)-Pen-AEF-2Nal-THP -E-N-3Pya-Sar-CONH2,
C14gEPEG2PEG2CO-Pen-N-T-7MeW-K (Ac)-Pen-AEF-2Nal-THP-E-N-3Pya-Sar-K (PEG2PEG2gEC14)-CONH2,
C12gEPEG2PEG2CO-Pen-N-T-7MeW-K (Ac)-Pen-AEF-2Nal-THP-E-N-3Pya-Sar-CONH2,
C14gEPEG2PEG2CO-Pen-N-T-7MeW-K (Ac)-Pen-AEF-2Nal-THP-E-N-3Pya-Sar-CONH2,
C12gEPEG2PEG2CO-Pen-K (PEG2PEG2gEC12)-T-7MeW-K (Ac)-Pen-AEF-2Nal-THP-E-N-3Pya-Sar-CONH2,
C14gEPEG2PEG2CO-Pen-K (PEG2PEG2gEC14)-T-7MeW-K (Ac)-Pen-AEF-2Nal-THP-E-N-3Pya-Sar-CONH2,
C12gEPEG2PEG2CO-Pen-N-T-7MeW-K (Ac)-Pen-AEF-2Nal-THP-K (PEG2PEG2gEC12)-N-3Pya-Sar-CONH2,
C14gEPEG2PEG2CO-Pen-N-T-7MeW-K (Ac)-Pen-AEF-2Nal-THP-K (PEG2PEG2gEC14)-N-3Pya-Sar-CONH2,
MeCO-k (PEG2PEG2gEC12)-Pen-N-T-7MeW-K (Ac)-Pen-AEF-2Nal-THP-E-N-3Pya-Sar-CONH2,
MeCO-k (PEG2PEG2gEC14)-Pen-N-T-7MeW-K (Ac)-Pen-AEF-2Nal-THP-E-N-3Pya-Sar-CONH2,
MeCO-r-Pen-N-T-7MeW-K (PEG2PEG2gEC12)-Pen-AEF-2Nal-THP-E-N-3Pya-Sar-K (PEG2PEG2gEC12)-CONH2,
MeCO-r-Pen-N-T-7MeW-K (PEG2PEG2gEC12)-Pen-AEF-2Nal-THP-K (PEG2PEG2gEC12)-N-3Pya-Sar-CONH2,
C12gEPEG2PEG2CO-r-Pen-N-T-7MeW-K (Ac)-Pen-AEF-2Nal-THP-E-N-3Pya-Sar-CONH2,
C12gEPEG2PEG2CO-r-Pen-N-T-7MeW-K (PEG2PEG2gEC12)-Pen-AEF-2Nal-THP-E-N -3Pya-Sar-CONH2,
C12gEPEG2PEG2CO-r-Pen-N-T-7MeW-K (Ac)-Pen-AEF-2Nal-THP-E-N-3Pya-Sar-K (PEG2PEG2gEC12)-CONH2,
MeCO-r-Pen-N-T-7MeW-K (Ac)-Pen-AEF-2Nal-THP-K (PEG2PEG2gEC12)-N-3Pya-Sar-CONH2,
C12gEPEG2PEG2CO-r-Pen-N-T-7MeW-K (Ac)-Pen-AEF-2Nal-THP-K (PEG2PEG2gEC12)-N-3Pya-Sar-CONH2,
MeCO-r-Pen-N-T-7MeW-K (PEG2PEG2gEC12)-Pen-AEF-2Nal-THP-E-N-3Pya-Sar-CONH2,
MeCO-r-Pen-N-T-7MeW-K (Ac)-Pen-AEF-2Nal-THP-E-N-3Pya-Sar-K (PEG2PEG2gEC12)-CONH2,
C12gEPEG2PEG2CO-Pen-N-T-7MeW-K (Ac)-Pen-AEF (G)-2Nal-THP-E-N-3Pya-Sar-CONH2,
cPEG3MeCOO-k (PEG2PEG2PEG2PEG2gEC12)-Pen-N (N (Me) 2)-T-7MeW-K (Ac)-Pen -TMAPF-2Nal-THP-K (NMeAc)-N-3Pya-NMeK (PEG2PEG2PEG2PEG2gEC12)-CONH2,
MeCO-Pen-N-T-7MeW-K (PEG2PEG2gEC14)-Pen-AEF-2Nal-THP-E-N-3Pya-Sar-CONH2,
MeCO-Pen-K (PEG2PEG2gEC14)-T-7MeW-K (Ac)-Pen-AEF-2Nal-THP-K (Ac)-N-3Pya-Sar-CONH2,
MeCO-Pen-N-T-7MeW-K (PEG2PEG2gEC12)-Pen-AEF-2Nal-THP-K (Ac)-N-3Pya-Sar-CONH2,
MeCO-Pen-N-T-7MeW-Pen-AEF-2Nal-THP-K (PEG2PEG2gEC12)-N-3Pya-Sar-CONH2,
MeCO-Pen-N-T-7MeW-K (Ac)-Pen-AEF-2Nal-THP-K (PEG2PEG2gEC14)-N-3Pya-Sar-CONH2,
MeCO-Pen-K (PEG2PEG2gEC12)-T-7MeW-K (Ac)-Pen-AEF-2Nal-THP-K (Ac)-N-3Pya-Sar-CONH2,
MeCO-Pen-N-T-7MeW-K (PEG2PEG2gEC14)-Pen-AEF-2Nal-THP-K (Ac)-N-3Pya-Sar-CONH2,
MeCO-Pen-N-T-7MeW-K (Ac)-Pen-AEF-2Nal-THP-K (PEG2PEG2gEC12)-N-3Pya-Sar-CONH2,
MeCO-r-Abu-N-T-7MeW-K (Ac)-aMeC-AEF-2Nal-THP-K (PEG2PEG2gEC12)-N-3Pya-Sar-CONH2,
MeCO-Pen-K (PEG2PEG2gEC14)-T-7MeW-K (Ac)-Pen-AEF-2Nal-THP-E-N-3Pya-Sar-CONH2,
MeCO-Pen-N-T-7MeW-K (PEG2PEG2gEC12)-Pen-AEF-2Nal-THP-E-N-3Pya-Sar-CONH2,
MeCO-r-Abu-N-T-W-K (PEG2PEG2gEC12)-aMeC-AEF-2Nal-THP-E-N-3Pya-Sar-CONH2,
MeCO-r-Abu-N-T-W-K (Ac)-aMeC-AEF-2Nal-THP-K (PEG2PEG2gEC12)-N-3Pya-Sar-CONH2,
MeCO-r-Abu-N-T-W-K (Ac)-aMeC-AEF-2Nal-THP-E-N-3Pya-Sar-K (PEG2PEG2gEC12)-CONH2,
MeCO-r-Abu-N-T-7MeW-K (PEG2PEG2gEC12)-aMeC-AEF-2Nal-THP-E-N-3Pya-Sar- CONH2,

MeCO-r-Abu-N-T-7MeW-K (Ac)-aMeC-AEF-2Nal-THP-E-N-3Pya-Sar- K (PEG2PEG2gEC12)-CONH2,

MeCO-Pen-N-T-7MeW-K (Ac)-Pen-AEF (G)-2Nal-THP-K (PEG2PEG2gEC14)-N-3Pya- Sar-CONH2,

C12gEPEG2PEG2CO-Pen-N-T-7MeW-K (Ac)-Pen-AEF-2Nal-THP-K (Ac)-N-3Pya-Sar- CONH2,

C14gEPEG2PEG2CO-Pen-N-T-7MeW-K (Ac)-Pen-AEF-2Nal-THP-K (Ac)-N-3Pya-Sar- CONH2,

MeCO-Pen-N-T-7MeW-K (Ac)-Pen-AEF-2Nal-THP-E-N-3Pya-Sar- K (PEG2PEG2gEC12)-CONH2,

MeCO-Pen-N-T-7MeW-K (Ac)-Pen-AEF-2Nal-THP-E-N-3Pya-Sar- K (PEG2PEG2gEC14)-CONH2,

MeCO-Pen-K (PEG2PEG2gEC12)-T-7MeW-K (Ac)-Pen-AEF-2Nal-THP-E-N-3Pya-Sar- CONH2,

MeCO-r-Pen-N-T-7MeW-K (Ac)-Pen-AEF-2Nal-THP-E-N-3Pya-Sar- K (PEG2NMePEG2NMegENMeC18Tetrazole)-CONH2,

MeCO-Pen-K (C14)-T-7MeW-K (Ac)-Pen-AEF-2Nal-THP-E-N-3Pya-Sar-CONH2, MeCO-Pen-N-T-7MeW-K (C14)-Pen-AEF-2Nal-THP-E-N-3Pya-Sar-CONH2, MeCO-Pen-N-T-7MeW-K (Ac)-Pen-AEF-2Nal-THP-K (C14)-N-3Pya-Sar-CONH2, MeCO-Pen-K (gEC14)-T-7MeW-K (Ac)-Pen-AEF-2Nal-THP-E-N-3Pya-Sar-CONH2, MeCO-Pen-N-T-7MeW-K (gEC14)-Pen-AEF-2Nal-THP-E-N-3Pya-Sar-CONH2, MeCO-Pen-N-T-7MeW-K (Ac)-Pen-AEF-2Nal-THP-K (gEC14)-N-3Pya-Sar-CONH2, MeCO-Pen-N-T-7MeW-K (Ac)-Pen-AEF-2Nal-THP-K (Ac)-N-3Pya-Sar- K (PEG2PEG2gEC14)-CONH2, MeCO-Pen-N-T-7MeW-K (Ac)-Pen-AEF-2Nal-THP-E-N-3Pya-Sar-K (gEC14)-CONH2, MeCO-Pen-N-T-7MeW-K (Ac)-Pen-AEF-2Nal-THP-E-N-3Pya-Sar-K (C14)-CONH2, MeCO-Pen-N-T-7MeW-K (Ac)-Pen-AEF-2Nal-THP-E-N-3Pya-Sar-K (gEC12)-CONH2, MeCO-k (PEG2PEG2gEC12)-Pen-N-T-7MeW-K (Ac)-Pen-AEF-2Nal-THP-K (Ac)-N- 3Pya-Sar-CONH2,

MeCO-k (PEG2PEG2gEC14)-Pen-N-T-7MeW-K (Ac)-Pen-AEF-2Nal-THP-K (Ac)-N- 3Pya-Sar-CONH2,

MeCO-Pen-N-T-7MeW-K (Ac)-Pen-AEF-2Nal-THP-K (Ac)-N-3Pya-Sar-K (PEG2PEG2gEC12)-CONH2,
MeCO-Pen-N-T-7MeW-K (Ac)-Pen-AEF-2Nal-THP-E-N-3Pya-Sar-K (PEG2PEG2gEDProC14)-CONH2,
MeCO-Pen-N-T-7MeW-K (Ac)-Pen-AEF-2Nal-THP-E-N-3Pya-Sar-K (PEG2PEG2C14)-CONH2,
MeCO-Pen-N-T-7MeW-K (Ac)-Pen-AEF-2Nal-THP-E-N-3Pya-Sar-K (GSGSGSGC14)-CONH2,
MeCO-Pen-N-T-7MeW-K (Ac)-Pen-AEF-2Nal-THP-E-N-3Pya-Sar-K (PEG2PEG2SP6C14)-CONH2,
MeCO-Pen-N-T-7MeW-K (Ac)-Pen-AEF-2Nal-THP-E-N-3Pya-Sar-K (PEG2C14)-CONH2,
MeCO-Pen-N-T-7MeW-K (Ac)-Pen-AEF-2Nal-THP-E-N-3Pya-Sar-K (PEG2PEG2gESarC14)-CONH2,
MeCO-Pen-N-T-7MeW-K (Ac)-Pen-AEF-2Nal-THP-E-N-3Pya-Sar-K (PEG2PEG2gEProC14)-CONH2,
ClAcPEG4CO-r-Pen-N-T-7MeW-K (Ac)-Pen-AEF-2Nal-THP-E-N-3Pya-Sar-K (PEG2PEG2gEC18OH)-CONH2,
MeCO-hkMe3-Pen-N-T-7MeW-K (PEG2PEG2gEC18OH)-Pen-AEF-2Nal-THP-E-N-3Pya-Sar-CONH2,
HOC18gEPEG2PEG2CO-hkMe3-Pen-N-T-7MeW-K (Ac)-Pen-AEF-2Nal-THP-E-N-3Pya-Sar-CONH2,
MeCO-hkMe3-Pen-N-T-7MeW-K (Ac)-Pen-AEF-2Nal-THP-K (PEG2PEG2gEC18OH)-N -3Pya-Sar-CONH2,
MeCO-hkMe3-Pen-K (PEG2PEG2gEC18OH)-T-7MeW-K (Ac)-Pen-AEF-2Nal-THP-E-N -3Pya-Sar-CONH2,
MeCO-hkMe3-Pen-N-T-7MeW-K (Ac)-Pen-AEF-2Nal-THP-E-N-3Pya-Sar-K (PEG2PEG2gEC18OH)-CONH2,
MeCO-kdCar-Pen-N-T-7MeW-K (Ac)-Pen-AEF-2Nal-THP-E-N-3Pya-Sar-K (PEG2PEG2gEC18OH)-CONH2,
MeCO-kdCar-Pen-N-T-7MeW-K (Ac)-Pen-AEF-2Nal-THP-K (PEG2PEG2gEC18OH)-N -3Pya-Sar-CONH2,
MeCO-kdCar-Pen-N-T-7MeW-K (Ac)-Pen-AEF-2Nal-THP-E-N-3Pya-NMeK (PEG2PEG2gEC18OH)-CONH2,
HOC18gEPEG2PEG2CO-kdCar-Pen-N-T-7MeW-K (Ac)-Pen-AEF-2Nal-THP-E-N-3Pya-Sar-CONH2,
HOC20gEPEG2PEG2CO-kdCar-Pen-N-T-7MeW-K (Ac)-Pen-AEF-2Nal-THP-E-N-3Pya-Sar-CONH2,
HOC20gEPEG2PEG2CO-kdCar-Pen-N-T-7MeW-K (Ac)-Pen-AEF-2Nal-THP-K (NMeAc)-N-3Pya-Sar-CONH2,
MeCO-kSP6-Pen-N-T-7MeW-K (Ac)-Pen-AEF-2Nal-THP-E-N-3Pya-Sar-K (PEG2PEG2gEC20OH)-CONH2,
MeCO-dOrn-Pen-N-T-7MeW-K (Ac)-Pen-AEF-2Nal-THP-E-N-3Pya-Sar-K (PEG2PEG2gEC18OH)-CONH2,
MeCO-kCar-Pen-N-T-7MeW-K (Ac)-Pen-AEF-2Nal-THP-E-N-3Pya-Sar-K (PEG2PEG2gEC18OH)-CONH2,
MeCO-dOrn-Pen-N-T-7MeW-K (Ac)-Pen-AEF-2Nal-THP-E-N-3Pya-Sar-K (PEG2PEG2gEC20OH)-CONH2,
MeCO-kCar-Pen-N-T-7MeW-K (Ac)-Pen-AEF-2Nal-THP-E-N-3Pya-Sar-K (PEG2PEG2gEC20OH)-CONH2,
MeCO-kdCar-Pen-K (PEG2PEG2gEC18OH)-T-7MeW-K (Ac)-Pen-AEF-2Nal-THP-E -N-3Pya-Sar-CONH2,
HOC18gEPEG2PEG2CO-kSP6-Pen-N-T-7MeW-K (Ac)-Pen-AEF-2Nal-THP-E-N -3Pya-Sar-CONH2,
MeCO-kdCar-Pen-N-T-7MeW-K (Ac)-Pen-AEF (PEG2PEG2gEC18OH)-2Nal-THP-E-N -3Pya-Sar-CONH2.

In some embodiments, the present disclosure provides a peptide of Formula (I), comprising the amino acid sequence:

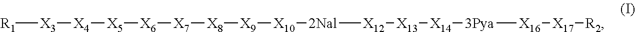

or a pharmaceutically acceptable salt thereof, wherein:
R₁ is MeCO, Z_{peg}, Z_{lipid}, succiniccarn, 5cpaCO, or AEEP-Z_{lipid};
X₃ is R, K—Z_{lipid}, Dab-Z_{lipid}, NMeK—Z_{lipid}, or absent;
X₄ is 4AminoPro, Abu, aG, aMeC, C, Dap, Pen, Pen(oXyl), Pen(mXyl), Pen(pXyl), or Pra;
X₅ is N, N(NMe₂), K(d), K—Z_{lipid}, Dab-Z_{lipid}, or NMeK—Z_{lipid};
X₆ is T, K—Z_{lipid}, NMeK—Z_{lipid}, or Dab-Z_{lipid};
X₇ is 7MeW, W, or absent;
X₈ is K(Ac), K(d), K(NMeAc), Q, K—Z_{lipid}, K—Z_{peg}, NMeK—Z_{lipid}, Dab-Z_{lipid}, Dab(NMecarn), or Dab-Z_{peg};
X₉ is aMeC, aG, C, D, E, hE, Pen, Dap, or Dap(N3);
X₁₀ is AEF, TMAPF, AEF(d), TMAPF-Z_{lipid}, APEG3F, or AEF-Z_{lipid};
X₁₂ is THP, K—Z_{lipid}, Dab-Z_{lipid}, or NMeK—Z_{lipid};
X₁₃ is E, K(Ac), K(d), K—Z_{peg}, K(NMeAc), Dab(NMecarn), E(OAll), K—Z_{lipid}, Dab-Z_{lipid}, NMeK—Z_{lipid}, or absent;
X₁₄ is N, K—Z_{lipid}, NMeK—Z_{lipid}, or Dab-Z_{lipid}
X₁₆ is Sar, NMeK(d), K—Z_{lipid}, Dab-Z_{lipid}, or NMeK—Z_{lipid};
X₁₇ is K—Z_{lipid}, Dab-Z_{lipid}, NMeK—Z_{lipid}, or absent;
R₂ is CONH2, CONMe2, CONH—Z_{peg}, or CO—Z_{lipid};
Z_{peg}, independently for each occurrence, is a polyethylene glycol chain;
Z_{lipid}, independently for each occurrence, is a lipophilic substituent;
wherein the peptide comprises a linkage between the residues at X₄ and X₉; and
wherein the polypeptide comprises at least one lipophilic substituent;
provided that the polypeptide is not selected from the group consisting of:
MeCO-r-Pen-N-T-7MeW—K(Ac)-Pen-AEF-2Nal-THP-E-N-3Pya-Sar-K(PEG2PEG2gEC18OH)—CONH2,
MeCO-r-Pen-N-T-7MeW—K(Ac)-Pen-AEF-2Nal-THP-E-N-3Pya-NmeK(PEG2PEG2gEC18OH)—CONH2,
MeCO-Pen-N-T-7MeW—K(Ac)-Pen-AEF-2Nal-THP-K(PEG2PEG2gEC18OH)—N-3Pya-Sar-CONH2,
MeCO-k(PEG2PEG2gEC18OH)-Pen-N-T-7MeW—K(Ac)-Pen-AEF-2Nal-THP-K(Ac)—N-3Pya-Sar-CONH2,
MeCO-r-Pen-N-T-7MeW—K(Ac)-Pen-AEF(PEG2PEG2gEC18OH)-2Nal-THP-K(Ac)—N-3Pya-Sar-CONH2,
MeCO-k(PEG2PEG2SP6gEC18OH)-Pen-N-T-7MeW—K(Ac)-Pen-AEF-2Nal-THP-K(Ac)—N-3Pya-Sar-CONH2,
MeCO-k(PEG2PEG2gEmXOH)-Pen-N-T-7MeW—K(Ac)-Pen-AEF-2Nal-THP-K(PEG2PEG2gEmXOH)—N-3Pya-Sar-CONH2,
MeCO-k(PEG2PEG2gEC16OH)-Pen-N-T-7MeW—K(Ac)-Pen-AEF-2Nal-THP-K(gEC16)-N-3Pya-Sar-CONH2,
HOC18gEPEG2PEG2-r-Pen-N-T-7MeW—K(Ac)-Pen-AEF-2Nal-THP-K(Ac)—N-3Pya-Sar-CONH2,
HOC18gEPEG2PEG2-r-Pen-N-T-7MeW—K(Ac)-Pen-AEF-2Nal-THP-K(PEG2PEG2gEC18OH)—N-3Pya-Sar-CONH2,
MeCO-k(PEG2PEG2gEDab(mXOH)2)-Pen-N-T-7MeW—K(Ac)-Pen-AEF-2Nal-THP-K(Ac)—N-3Pya-Sar-CONH2,
MeCO-k(PEG2PEG2gE(c)C18OH)-Pen-N-T-7MeW—K(Ac)-Pen-AEF-2Nal-THP-K(Ac)—N-3Pya-Sar-CONH2,
MeCO-r-Pen-N-T-7MeW—K(Ac)-Pen-AEF-2Nal-THP-E-N-3Pya-Sar-K(PEG2PEG2gEC16OH)—CONH2,
MeCO-r-Pen-N-T-7MeW—K(Ac)-Pen-AEF-2Nal-THP-E-N-3Pya-K(PEG2PEG2gEC18OH)—CONH2,
MeCO-Pen-N-T-7MeW—K(Ac)-Pen-AEF-2Nal-THP-K(PEG2PEG2gEC16OH)—N-3Pya-Sar-CONH2,
MeCO-Pen-K(PEG2PEG2gEC18OH)-T-7MeW—K(Ac)-Pen-AEF-2Nal-THP-E-N-3Pya-Sar-CONH2,
MeCO-r-Pen-K(PEG2PEG2gEC16OH)-T-7MeW—K(Ac)-Pen-AEF-2Nal-THP-E-N-3Pya-Sar-CONH2,
MeCO-r-Pen-N-T-7MeW—K(Ac)-Pen-AEF-2Nal-THP-E-N-3Pya-NMeK(PEG2PEG2gEC16OH)—CONH2,
MeCO-r-Pen-N-T-7MeW—K(Ac)-Pen-AEF-2Nal-THP-E-N-3Pya-K(PEG2PEG2gEC16OH)—CONH2,
MeCO-k(PEG2PEG2gEC16OH)-Pen-N-T-7MeW—K(Ac)-Pen-AEF-2Nal-THP-K(Ac)—N-3Pya-Sar-CONH2,
MeCO-r-Pen-N-T-7MeW—K(Ac)-Pen-AEF(PEG2PEG2gEC16OH)-2Nal-THP-K(Ac)—N-3Pya-Sar-CONH2,
MeCO-r-Pen-N-T-7MeW—K(Ac)-Pen-AEF-2Nal-THP-K(PEG2PEG2gEC18OH)—N-3Pya-Sar-CONH2,
MeCO-r-Pen-N-T-7MeW—K(Ac)-Pen-AEF-2Nal-THP-K(PEG2PEG2gEC16OH)—N-3Pya-Sar-CONH2,
MeCO-Pen-N-T-7MeW—K(Ac)-Pen-AEF-2Nal-THP-K(gEC16OH)—N-3Pya-Sar-CONH2,
MeCO-Pen-N-T-7MeW—K(Ac)-Pen-AEF-2Nal-THP-K(gEC18OH)—N-3Pya-Sar-CONH2,
MeCO-Pen-N-T-7MeW—K(Ac)-Pen-AEF-2Nal-THP-K(gEC18)-N-3Pya-Sar-CONH2,
MeCO-Pen-N-T-7MeW—K(Ac)-Pen-AEF-2Nal-THP-K(PEG6gEC16OH)—N-3Pya-Sar-CONH2,
MeCO-Pen-N-T-7MeW—K(Ac)-Pen-AEF-2Nal-THP-K(PEG6gEC18OH)—N-3Pya-Sar-CONH2,
MeCO-Pen-N-T-7MeW—K(Ac)-Pen-AEF-2Nal-THP-K(PEG2gEC16OH)—N-3Pya-Sar-CONH2,
MeCO-Pen-N-T-7MeW—K(Ac)-Pen-AEF-2Nal-THP-K(PEG2gEC18OH)—N-3Pya-Sar-CONH2,
MeCO-Pen-N-T-7MeW—K(Ac)-Pen-AEF-2Nal-THP-K(PEG2PEG2gEC20OH)—N-3Pya-Sar-CONH2,
HOC16gEPEG2PEG2-r-Pen-N-T-7MeW—K(Ac)-Pen-AEF-2Nal-THP-K(PEG2PEG2gEC16OH)—N-3Pya-Sar-CONH2,
MeCO-Pen-N-T-7MeW—K(Ac)-Pen-AEF-2Nal-THP-K(PEG2PEG6gEC16OH)—N-3Pya-Sar-CONH2,
MeCO-Pen-N-T-7MeW—K(Ac)-Pen-AEF-2Nal-THP-K(PEG2PEG6gEC18OH)—N-3Pya-Sar-CONH2,
MeCO-Pen-N-T-7MeW—K(Ac)-Pen-AEF-2Nal-THP-K(PEG2PEG2C16OH)—N-3Pya-Sar-CONH2,
MeCO-Pen-N-T-7MeW—K(Ac)-Pen-AEF-2Nal-THP-K(PEG2PEG2C18OH)—N-3Pya-Sar-CONH2,
MeCO-Pen-N-T-7MeW—K(Ac)-Pen-AEF-2Nal-THP-K(PEG6PEG6gEC16OH)—N-3Pya-Sar-CONH2,
MeCO-Pen-N-T-7MeW—K(Ac)-Pen-AEF-2Nal-THP-K(PEG6PEG6gEC18OH)—N-3Pya-Sar-CONH2,
MeCO-Pen-N-T-7MeW—K(Ac)-Pen-AEF-2Nal-THP-K(PEG24-gEC16OH)—N-3Pya-Sar-CONH2,
MeCO-Pen-N-T-7MeW—K(Ac)-Pen-AEF-2Nal-THP-K(PEG24-gEC18OH)—N-3Pya-Sar-CONH2,
MeCO-Pen-N-T-7MeW—K(Ac)-Pen-AEF-2Nal-THP-K(PEG24-C18OH)—N-3Pya-Sar-CONH2,
MeCO-k(gEC18OH)-Pen-N-T-7MeW—K(Ac)-Pen-AEF-2Nal-THP-K(Ac)—N-3Pya-Sar-CONH2,
MeCO-k(PEG2gEC18OH)-Pen-N-T-7MeW—K(Ac)-Pen-AEF-2Nal-THP-K(Ac)—N-3Pya-Sar-CONH2,
MeCO-r-Pen-K(PEG2PEG2gEC18OH)-T-7MeW—K(Ac)-Pen-AEF-2Nal-THP-K(Ac)—N-3Pya-Sar-CONH2,
MeCO-r-Pen-K(gEC18OH)-T-7MeW—K(Ac)-Pen-AEF-2Nal-THP-K(Ac)—N-3Pya-Sar-CONH2,
MeCO-r-Pen-K(PEG2gEC18OH)-T-7MeW—K(Ac)-Pen-AEF-2Nal-THP-K(Ac)—N-3Pya-Sar-CONH2,
MeCO-r-Pen-K(PEG2PEG2C18OH)-T-7MeW—K(Ac)-Pen-AEF-2Nal-THP-K(Ac)—N-3Pya-Sar-CONH2,
MeCO-r-Pen-N-T-7MeW—K(Ac)-Pen-AEF-2Nal-THP-K(Ac)—N-3Pya-NMeK(PEG2PEG2gEC18OH)—CONH2,
MeCO-r-Pen-N-T-7MeW—K(Ac)-Pen-AEF-2Nal-THP-K(PEG2PEG2-DgEC18OH)—N-3Pya-Sar-CONH2,
MeCO-r-Pen-N-T-7MeW—K(Ac)-Pen-AEF-2Nal-THP-K(PEG2PEG2-pC18OH)—N-3Pya-Sar-CONH2,
MeCO-r-Pen-N-T-7MeW—K(Ac)-Pen-AEF-2Nal-THP-K(PEG2PEG2gETrxC18OH)—N-3Pya-Sar-CONH2,
MeCO-r-Pen-N-T-7MeW—K(Ac)-Pen-AEF-2Nal-THP-K(PEG2PEG2gETrxC20OH)—N-MeCO-k(PEG6gEC18OH)-Pen-N-T-7MeW—K(Ac)-Pen-AEF-2Nal-THP-K(Ac)—N-3Pya-Sar-CONH2,
MeCO-k(PEG2PEG6gEC18OH)-Pen-N-T-7MeW—K(Ac)-Pen-AEF-2Nal-THP-K(Ac)—N-3Pya-Sar-CONH2,
MeCO-k(PEG2PEG2C18OH)-Pen-N-T-7MeW—K(Ac)-Pen-AEF-2Nal-THP-K(Ac)—N-3Pya-Sar-CONH2,
MeCO-r-Pen-K(PEG6gEC18OH)-T-7MeW—K(Ac)-Pen-AEF-2Nal-THP-K(Ac)—N-3Pya-Sar-CONH2,
MeCO-r-Pen-K(PEG2PEG6gEC18OH)-T-7MeW—K(Ac)-Pen-AEF-2Nal-THP-K(Ac)—N-3Pya-Sar-CONH2,
MeCO-r-Pen-N-T-7MeW—K(Ac)-Pen-AEF-2Nal-THP-K(PEG2PEG2-pppC18OH)—N-3Pya-Sar-CONH2,
MeCO-k(gEC16)-Pen-N-T-7MeW—K(Ac)-Pen-AEF-2Nal-THP-K(Ac)—N-3Pya-Sar-CONH2,
MeCO-k(gEC18)-Pen-N-T-7MeW—K(Ac)-Pen-AEF-2Nal-THP-K(Ac)—N-3Pya-Sar-CONH2,
MeCO-r-Pen-K(gEC16)-T-7MeW—K(Ac)-Pen-AEF-2Nal-THP-K(Ac)—N-3Pya-Sar-CONH2,
MeCO-r-Pen-K(gEC18)-T-7MeW—K(Ac)-Pen-AEF-2Nal-THP-K(Ac)—N-3Pya-Sar-CONH2,
MeCO-k(PEG2PEG2gETrxC18OH)-Pen-N-T-7MeW—K(Ac)-Pen-AEF-2Nal-THP-K(Ac)—N-3Pya-Sar-CONH2,
MeCO-k(PEG2PEG2gETrxC20OH)-Pen-N-T-7MeW—K(Ac)-Pen-AEF-2Nal-THP-K(Ac)—N-3Pya-Sar-CONH2,
MeCO-Pen-K(PEG2PEG2gETrxC18OH)-T-7MeW—K(Ac)-Pen-AEF-2Nal-THP-K(Ac)—N-3Pya-Sar-CONH2,
MeCO-Pen-K(PEG2PEG2gETrxC20OH)-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-K(gEC16)-N-3Pya-Sar-CONH2,
MeCO-k(PEG2PEG2gEC16OH)-Pen-K(PEG2PEG2gEC16OH)-T-7MeW—K(Ac)-Pen-AEF-2Nal-THP-K(Ac)—N-3Pya-Sar-CONH2,
MeCO-k(PEG2PEG2gEC18OH)-Pen-K(PEG2PEG2gEC18OH)-T-7MeW—K(Ac)-Pen-AEF-2Nal-THP-K(Ac)—N-3Pya-Sar-CONH2,
MeCO-k(PEG2PEG2gEC20OH)-Pen-K(PEG2PEG2gEC20OH)-T-7MeW—K(Ac)-Pen-AEF-2Nal-THP-K(Ac)—N-3Pya-Sar-CONH2,
MeCO-k(gEC16)-Pen-K(gEC16)-T-7MeW—K(Ac)-Pen-AEF-2Nal-THP-K(Ac)—N-3Pya-Sar-CONH2,
MeCO-k(gEC18)-Pen-K(gEC18)-T-7MeW—K(Ac)-Pen-AEF-2Nal-THP-K(Ac)—N-3Pya-Sar-CONH2,
MeCO-k(PEG2PEG2gEC16)-Pen-N-T-7MeW—K(Ac)-Pen-AEF-2Nal-THP-K(Ac)—N-3Pya-Sar-CONH2,
MeCO-k(PEG2PEG2gEC18)-Pen-N-T-7MeW—K(Ac)-Pen-AEF-2Nal-THP-K(Ac)—N-3Pya-Sar-CONH2,
MeCO-Pen-K(PEG2PEG2gEC16)-T-7MeW—K(Ac)-Pen-AEF-2Nal-THP-K(Ac)—N-3Pya-Sar-CONH2,
MeCO-Pen-K(PEG2PEG2gEC18)-T-7MeW—K(Ac)-Pen-AEF-2Nal-THP-K(Ac)—N-3Pya-Sar-CONH2,
MeCO-Pen-K(1PEG21PEG2IsoGluC16Diacid)-T-7MeW—K(Ac)-Pen-AEF-2Nal-TIP—K(Ac)—N-3Pya-Sar-CONH2,
MeCO-r-Pen-N-T-7MeW—K(PEG2PEG2gEC18OH)-Pen-AEF-2Nal-THP-K(Ac)—N-3Pya-Sar-CONH2,
MeCO-Pen-N-T-7MeW—K(Ac)-Pen-AEF-2Nal-THP-K(gEC20OH)—N-3Pya-Sar-CONH2,
MeCO-Pen-N-T-7MeW—K(Ac)-Pen-AEF-2Nal-THP-K(PEG2PEG2-TrxgEC18OH)—N-3Pya-Sar-CONH2,
MeCO-Pen-N-T-7MeW—K(Ac)-Pen-AEF-2Nal-THP-K(PEG2PEG2-TrxgETrxC20OH)—N-3Pya-Sar-CONH2,
MeCO-Pen-N-T-7MeW—K(Ac)-Pen-AEF-2Nal-THP-K(PEG2PEG2-TrxgEC20OH)—N-3Pya-Sar-CONH2,
MeCO-k(PEG2PEG2gEC10OH)-Pen-N-T-7MeW—K(Ac)-Pen-AEF-2Nal-THP-K(gEC16)-N-3Pya-Sar-CONH2,
MeCO-k(PEG2PEG2gEC18OH)-Pen-K(gEC16)-T-7MeW—K(Ac)-Pen-AEF-2Nal-THP-K(Ac)—N-3Pya-Sar-CONH2,
MeCO-r-Pen-K(PEG2PEG2pgEC18OH)-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-K(PEG2PEG2gEmXOH)—N-3Pya-Sar-CONH2,
MeCO-k(PEG2PEG2gEC18OH)-Pen-N-T-7MeW—K(Ac)-Pen-AEF-2Nal-THP-K(gEC16)-N-3Pya-Sar-CONH2,
MeCO-r-Pen-K(PEG2PEG2pppgEC18OH)-T-7MeW—K(Ac)-Pen-AEF-2Nal-THP-K(Ac)—N-3Pya-Sar-CONH2,
MeCO-k(PEG2PEG2PgEC18OH)-Pen-N-T-7MeW—K(Ac)-Pen-AEF-2Nal-THP-K(Ac)—N-3Pya-Sar-CONH2,
MeCO-k(PEG2PEG2PPPgEC18OH)-Pen-N-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-K(PEG2PEG2PgEC18OH)—N-3Pya-Sar-CONH2,
MeCO-Pen-N-T-7MeW—K(Ac)-Pen-AEF-2Nal-THP-K(PEG2PEG2PPPgEC18OH)—N-3Pya-Sar-CONH2,
MeCO-k(PEG2PEG2-TrxgEC18OH)-Pen-N-T-7MeW—K(Ac)-Pen-AEF-2Nal-THP-K(Ac)—N-3Pya-Sar-CONH2,
MeCO-k(PEG2PEG2gEmXOH)-Pen-N-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-K(PEG2PEG2gEpXOH)—N-3Pya-Sar-CONH2,
MeCO-k(PEG2PEG2gEpXOH)-Pen-N-T-7MeW—K(Ac)-Pen-AEF-2Nal-THP-K(PEG2PEG2gEpXOH)—N-3Pya-Sar-CONH2,
MeCO-k(PEG2PEG2gEmXOH)-Pen-N-T-7MeW—K(Ac)-Pen-AEF-2Nal-THP-K(gEC16)-N-3Pya-Sar-CONH2,
MeCO-Pen-N-T-7MeW—K(Ac)-Pen-AEF-2Nal-THP-K(DAP(C16OH)2)-N-3Pya-Sar-CONH2,
MeCO-k(DAP(C16OH)2)-Pen-N-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-K(PEG2PEG2gEDAP(C16OH)2)-N-3Pya-Sar-CONH2,
MeCO-k(PEG2PEG2gEDAP(C16OH)2)-Pen-N-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-K(PEG2PEG2Sp6gEC18OH)—N-3Pya-Sar-CONH2,
MeCO-Pen-N-T-7MeW—K(Ac)-Pen-AEF-2Nal-THP-K(PEG2Sp6PEG2gEC18OH)—N-3Pya-Sar-CONH2,
MeCO-k(PEG2PEG2gEC16OH)-Pen-N-T-7MeW—K(Ac)-Pen-AEF-2Nal-THP-K(gEC18)-N-3Pya-Sar-CONH2,
MeCO-k(PEG2PEG2gEC18OH)-Pen-N-T-7MeW—K(Ac)-Pen-AEF-2Nal-THP-K(gEC18)-N-3Pya-Sar-CONH2,
MeCO-Pen-K(1PEG21PEG2IsoGluC18Diacid)-T-7MeW—K(Ac)-Pen-AEF-2Nal-THP-K(Ac)—N-3Pya-Sar-CONH2,
MeCO-Pen-K(PEG2PEG2gEC18OH)-T-7MeW—K(Ac)-Pen-AEF-2Nal-THP-K(PEG2PEG2gESp6C18OH)—N-3Pya-Sar-CONH2,
MeCO-Pen-K(PEG2PEG2gEC18OH)-T-7MeW—K(Ac)-Pen-AEF-2Nal-THP-K(Sp6PEG2PEG2gEC18OH)—N-3Pya-Sar-CONH2,
MeCO-k(gEC16)-Pen-N-T-7MeW—K(Ac)-Pen-AEF-2Nal-THP-K(gEC18)-N-3Pya-Sar-CONH2,
MeCO-k(gEC18)-Pen-N-T-7MeW—K(Ac)-Pen-AEF-2Nal-THP-K(gEC18)-N-3Pya-Sar-CONH2,
MeCO-k(PEG2PEG2gEC10OH)-Pen-N-T-7MeW—K(Ac)-Pen-AEF-2Nal-THP-K(gEC18)-N-3Pya-Sar-CONH2,
MeCO-Pen-K(PEG2PEG2gEC18OH)-T-7MeW—K(Ac)-Pen-AEF-2Nal-THP-K(PEG2PEG2gETrxC20OH)—N-3Pya-Sar-CONH2,
MeCO-k(PEG2PEG2gESp6C18OH)-Pen-N-T-7MeW—K(Ac)-Pen-AEF-2Nal-THP-K(Ac)—N-3Pya-Sar-CONH2,
MeCO-k(PEG2Sp6PEG2gEC18OH)-Pen-N-T-7MeW—K(Ac)-Pen-AEF-2Nal-THP-K(Ac)—N-3Pya-Sar-CONH2,
MeCO-k(Sp6PEG2PEG2gEC18OH)-Pen-N-T-7MeW—K(Ac)-Pen-AEF-2Nal-THP-K(Ac)—N-3Pya-Sar-CONH2,
MeCO—K[PEG2PEG2-gEDAP(pXOH)2]-Pen-N-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-K[PEG2PEG2-gEDAP-(mXOH)2]—N-3Pya-Sar-CONH2,
MeCO-k(GolAC16)-Pen-N-T-7MeW—K(Ac)-Pen-AEF-2Nal-THP-K(Ac)—N-3Pya-Sar-CONH2,
MeCO-k(GolAC16OH)-Pen-N-T-7MeW—K(Ac)-Pen-AEF-2Nal-THP-K(Ac)—N-3Pya-Sar-CONH2,
MeCO-k(GolAC18OH)-Pen-N-T-7MeW—K(Ac)-Pen-AEF-2Nal-THP-K(Ac)—N-3Pya-Sar-CONH2,
MeCO-k(PEG2PEG2 GolAC18OH)-Pen-N-T-7MeW—K(Ac)-Pen-AEF-2Nal-THP-K(Ac)—N-3Pya-Sar-CONH2,
MeCO-k(PEG2PEG2-C18GolB)-Pen-N-T-7MeW—K(Ac)-Pen-AEF-2Nal-THP-K(Ac)—N-3Pya-Sar-CONH2,
MeCO-k(PEG2PEG2gE(C)C18OH)-Pen-N-T-7MeW—K(Ac)-Pen-AEF-2Nal-THP-K(Ac)—N-3Pya-Sar-CONH2,
MeCO-k(PEG2PEG2gEC18OH(C))-Pen-N-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-E-N-3Pya-Sar-k(PEG12_IsoGlu_C18_Diacid)-CONH2,
MeCO-Pen-N-T-7MeW—K(Ac)-Pen-AEF-2Nal-THP-E-N-3Pya-Sar-k(Peg4_IsoGlu_Palm)-CONH2,
MeCO-Pen-N-T-7MeW—K(Ac)-Pen-AEF-2Nal-THP-E-N-3Pya-Sar-k(IsoGlu_Palm)-CONH2,
MeCO-Pen-N-T-7MeW—K(Ac)-Pen-AEF-2Nal-TIP-E-N-3Pya-Sar-k(PEG12_C18_Diacid)-CONH2,
MeCO-Pen-N-T-7MeW—K(Ac)-Pen-AEF-2Nal-THP-E-N-3Pya-Sar-k(Peg4_IsoGlu_C18_Diacid)-CONH2,
MeCO-Pen-N-T-7MeW—K(Ac)-Pen-AEF-2Nal-THP-E-N-3Pya-Sar-k(PEG12_IsoGlu_Palm)-CONH2,
MeCO-Pen-N-T-7MeW—K(Ac)-Pen-AEF-2Nal-THP-E-N-3Pya-Sar-k(Peg4_C18_Diacid)-CONH2,
MeCO-Pen-N-T-7MeW—K(Ac)-Pen-AEF-2Nal-THP-E-N-3Pya-Sar-k(IsoGlu_C18_Diacid)-CONH2,
MeCO-r-Pen-K(1PEG21PEG2IsoGluC16Diacid)-T-7MeW—K(Ac)-Pen-AEF-2Nal-THP-E-N-3Pya-Sar-CONH2,
MeCO-r-Pen-K(1PEG21PEG2IsoGluC18Diacid)-T-7MeW—K(Ac)-Pen-AEF-2Nal-TIP-E-N-3Pya-Sar-CONH2,
MeCO-Pen-N-T-7MeW—K(Ac)-Pen-AEF-2Nal-TIP-K(1PEG21PEG2IsoGluC16Diacid)-N-3Pya-Sar-CONH2,
MeCO-Pen-N-T-7MeW—K(Ac)-Pen-AEF-2Nal-TIP-K(1PEG21PEG2IsoGluC18Diacid)-N-3Pya-Sar-CONH2,
MeCO-r-Pen-N-T-7MeW—K(Ac)-Pen-AEF-2Nal-THP-E-N-3Pya-K(1PEG21PEG2IsoGluC18Diacid)-CONH2,
MeCO-r-Pen-N-T-7MeW—K(Ac)-Pen-AEF-2Nal-THP-E-N-3Pya-Sar-K(1PEG21PEG2IsoGluC16Diacid)-CONH2,
MeCO-r-Pen-N-T-7MeW—K(Ac)-Pen-AEF-2Nal-THP-E-N-3Pya-Sar-K(1PEG21PEG2IsoGluC18Diacid)-CONH2,
HOC10gEPEG2COPEG2CO-r-Pen-N-T-7MeW—K(Ac)-Pen-AEF-2Nal-THP-K(PEG2PEG2gEC10OH)—N-3Pya-Sar-CONH2,
MeCO-Pen-N-T-7MeW—K(Ac)-Pen-AEF-2Nal-TIP-K(PEG2PEG2gEC10OH)—N-3Pya-Sar-CONH2,
MeCO-k(PEG2PEG2gEC10OH)-Pen-N-T-7MeW—K(Ac)-Pen-AEF-2Nal-THP-K(Ac)—N-3Pya-Sar-CONH2,
MeCO-Pen-K(PEG2PEG2gEC10OH)-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-K(gEC10OH)—N-3Pya-Sar-CONH2,
MeCO-k(PEG2PEG2gEC10OH)-Pen-K(PEG2PEG2gEC10OH)-T-7MeW—K(Ac)-Pen-AEF-2Nal-THP-K(Ac)—N-3Pya-Sar-CONH2,
HOC16gEPEG2PEG2orn(2)-r-Pen-N-T-7MeW—K(Ac)-Pen-AEF-2Nal-THP-E(2)-N-3Pya-Sar-CONH2,
MeCO-k(PEG2PEG2gEC12OH(c))-Pen-N-T-7MeW—K(Ac)-Pen-AEF-2Nal-THP-K(Ac)—N-3Pya-Sar-CONH2,
MeCO-k(PEG2PEG2gE(C)C₁₂)-Pen-N-T-7MeW—K(Ac)-Pen-AEF-2Nal-THP-K(Ac)—N-3Pya-Sar-CONH2,
MeCO-r-Pen-N-T-7MeW—K(Ac)-Pen-AEF-2Nal-THP-K(FITCPEG4)-N-3Pya-Sar-K(PEG2PEG2gEC18OH)—CONH2,
succiniccarn-k(PEG2PEG2gEC18OH)-Pen-N-T-7MeW—K(Ac)-Pen-AEF-2Nal-THP-E-N-3Pya-Sar-CONH2,
MeCO-r-Pen-K(PEG2PEG2gEC18OH)-T-7MeW—K(Ac)-Pen-dAEF-2Nal-THP-E-N-3Pya-Sar-CONH2,
MeCO-Pen-N-T-W—K(PEG2PEG2gEC20OH)-Pen-AEF-2Nal-THP-K(Ac)—N-3Pya-Sar-CONH2,
MeCO-Pen-K(PEG2PEG2gEC20OH)-T-W—K(Ac)-Pen-AEF-2Nal-THP-K(Ac)—N-3Pya-Sar-CONH2,
MeCO—K(PEG2PEG2gEC20OH)-Pen-N-T-W—K(Ac)-Pen-AEF-2Nal-THP-K(Ac)—N-3Pya-Sar-CONH2,
MeCO-Pen-N-T-W—K(Ac)-Pen-AEF-2Nal-THP-K(Ac)—N-3Pya-Sar-K(PEG2PEG2gEC20OH)—CONH2,
MeCO-r-Pen-N-T-7MeW—K(PEG2PEG2gEC20OH)-Pen-AEF-2Nal-THP-E-N-3Pya-Sar-CONH2,
cPEG3aCO-r-Pen-N-T-7MeW—K(PEG2PEG2gEC20OH)-Pen-AEF-2Nal-THP-E-N-3Pya-Sar-CONH2,
cPEG3aCO-r-Pen-K(PEG2PEG2gEC20OH)-T-7MeW—K(Ac)-Pen-AEF-2Nal-THP-E-N-3Pya-Sar-CONH2,
MeCO-r-Pen-N-T-7MeW—K(Ac)-Pen-AEF-2Nal-THP-E-N-3Pya-Sar-K(PEG2PEG2gEC20OH)—CONH2,
MeCO-r-Abu-N-T-7MeW—K(Ac)-aMeC-AEF-2Nal-THP-E-N-3Pya-Sar-K(PEG2PEG2gEC18OH)—CONH2,
MeCO-r-Abu-N-T-7MeW—K(Ac)-Pen-AEF-2Nal-THP-E-N-3Pya-Sar-K(PEG2PEG2gEC18OH)—CONH2,
MeCO-Pen-N-T-7MeW—K(PEG2PEG2gEC12)-Pen-AEF-2Nal-THP-K(PEG2PEG2gEC12)-N-3Pya-Sar-CONH2,
MeCO-Pen-N-T-7MeW—K(PEG2PEG2gEC14)-Pen-AEF-2Nal-THP-K(PEG2PEG2gEC14)-N-3Pya-Sar-CONH2,
MeCO-k(PEG2PEG2gEC12)-Pen-N-T-7MeW—K(Ac)-Pen-AEF-2Nal-THP-E-N-3Pya-Sar-CONH2,
MeCO-k(PEG2PEG2gEC14)-Pen-N-T-7MeW—K(Ac)-Pen-AEF-2Nal-THP-E-N-3Pya-Sar-CONH2,
MeCO-r-Pen-N-T-7MeW—K(PEG2PEG2gEC12)-Pen-AEF-2Nal-THP-E-N-3Pya-Sar-K(PEG2PEG2gEC12)-CONH2,
MeCO-r-Pen-N-T-7MeW—K(PEG2PEG2gEC12)-Pen-AEF-2Nal-THP-K(PEG2PEG2gEC12)-N-3Pya-Sar-CONH2,
MeCO-r-Pen-N-T-7MeW—K(Ac)-Pen-AEF-2Nal-THP-K(PEG2PEG2gEC12)-N-3Pya-Sar-CONH2,
MeCO-r-Pen-N-T-7MeW—K(PEG2PEG2gEC12)-Pen-AEF-2Nal-THP-E-N-3Pya-Sar-CONH2,
MeCO-r-Pen-N-T-7MeW—K(Ac)-Pen-AEF-2Nal-THP-E-N-3Pya-Sar-K(PEG2PEG2gEC12)-CONH2,
cPEG3aCO-k(PEG2PEG2PEG2PEG2gEC12)-Pen-N(N(Me)₂)-T-7MeW—K(Ac)-Pen-TMApF-2Nal-THP-K(nMeAc)—N-3Pya-NMeK(PEG2PEG2PEG2PEG2gEC12)-CONH2,
MeCO-Pen-N-T-7MeW—K(PEG2PEG2gEC14)-Pen-AEF-2Nal-THP-E-N-3Pya-Sar-MeCO-Pen-K(PEG2PEG2gEC14)-T-7MeW—K(Ac)-Pen-AEF-2Nal-THP-K(Ac)—N-3Pya-Sar-CONH2,
MeCO-Pen-N-T-7MeW—K(PEG2PEG2gEC12)-Pen-AEF-2Nal-THP-K(Ac)—N-3Pya-Sar-CONH2,
MeCO-Pen-N-T-7MeW—K(Ac)-Pen-AEF-2Nal-THP-K(PEG2PEG2gEC14)-N-3Pya-Sar-CONH2,
MeCO-Pen-K(PEG2PEG2gEC12)-T-7MeW—K(Ac)-Pen-AEF-2Nal-THP-K(Ac)—N-3Pya-Sar-CONH2,
MeCO-Pen-N-T-7MeW—K(PEG2PEG2gEC14)-Pen-AEF-2Nal-THP-K(Ac)—N-3Pya-Sar-CONH2,
MeCO-Pen-N-T-7MeW—K(Ac)-Pen-AEF-2Nal-THP-K(PEG2PEG2gEC12)-N-3Pya-Sar-CONH2,
MeCO-r-Abu-N-T-7MeW—K(Ac)-aMeC-AEF-2Nal-THP-K(PEG2PEG2gEC12)-N-3Pya-Sar-CONH2,
MeCO-Pen-K(PEG2PEG2gEC14)-T-7MeW—K(Ac)-Pen-AEF-2Nal-THP-E-N-3Pya-Sar-CONH2,
MeCO-Pen-N-T-7MeW—K(PEG2PEG2gEC12)-Pen-AEF-2Nal-THP-E-N-3Pya-Sar-CONH2,
MeCO-r-Abu-N-T-W—K(Ac)-aMeC-AEF-2Nal-THP-K(PEG2PEG2gEC12)-N-3Pya-Sar-CONH2,
MeCO-r-Abu-N-T-W—K(Ac)-aMeC-AEF-2Nal-THP-E-N-3Pya-Sar-K(PEG2PEG2gEC12)-CONH2,
MeCO-r-Abu-N-T-7MeW—K(PEG2PEG2gEC12)-aMeC-AEF-2Nal-THP-E-N-3Pya-Sar-CONH2,
MeCO-r-Abu-N-T-7MeW—K(Ac)-aMeC-AEF-2Nal-THP-E-N-3Pya-Sar-K(PEG2PEG2gEC12)-CONH2,
MeCO-r-Abu-N-T-W—K(PEG2PEG2gEC12)-aMeC-AEF-2Nal-THP-E-N-3Pya-Sar-CONH2,
MeCO-Pen-N-T-7MeW—K(Ac)-Pen-AEF(G)-2Nal-THP-K(PEG2PEG2gEC14)-N-3Pya-Sar-CONH2,
MeCO-Pen-N-T-7MeW—K(Ac)-Pen-AEF-2Nal-THP-E-N-3Pya-Sar-K(PEG2PEG2gEC12)-CONH2,
MeCO-Pen-N-T-7MeW—K(Ac)-Pen-AEF-2Nal-THP-E-N-3Pya-Sar-K(PEG2PEG2gEC14)-CONH2,
MeCO-Pen-K(PEG2PEG2gEC12)-T-7MeW—K(Ac)-Pen-AEF-2Nal-THP-E-N-3Pya-Sar-CONH2,
MeCO-r-Pen-N-T-7MeW—K(Ac)-Pen-AEF-2Nal-THP-E-N-3Pya-Sar-K(PEG2NMePEG2NMegENMeC18Tetrazole)-CONH2,
MeCO-Pen-K(C14)-T-7MeW—K(Ac)-Pen-AEF-2Nal-THP-E-N-3Pya-Sar-CONH2,
MeCO-Pen-N-T-7MeW—K(C14)-Pen-AEF-2Nal-THP-E-N-3Pya-Sar-CONH2,
MeCO-Pen-N-T-7MeW—K(Ac)-Pen-AEF-2Nal-THP-K(C14)-N-3Pya-Sar-CONH2,
MeCO-Pen-K(gEC14)-T-7MeW—K(Ac)-Pen-AEF-2Nal-THP-E-N-3Pya-Sar-CONH2,
MeCO-Pen-N-T-7MeW—K(gEC14)-Pen-AEF-2Nal-THP-E-N-3Pya-Sar-CONH2,
MeCO-Pen-N-T-7MeW—K(Ac)-Pen-AEF-2Nal-THP-K(gEC14)-N-3Pya-Sar-CONH2,
MeCO-Pen-N-T-7MeW—K(Ac)-Pen-AEF-2Nal-THP-K(Ac)—N-3Pya-Sar-K(PEG2PEG2gEC14)-CONH2,
MeCO-Pen-N-T-7MeW—K(Ac)-Pen-AEF-2Nal-THP-E-N-3Pya-Sar-K(gEC14)-CONH2,
MeCO-Pen-N-T-7MeW—K(Ac)-Pen-AEF-2Nal-THP-E-N-3Pya-Sar-K(gEC12)-CONH2,
MeCO-k(PEG2PEG2gEC12)-Pen-N-T-7MeW—K(Ac)-Pen-AEF-2Nal-THP-K(Ac)—N-3Pya-Sar-CONH2,
MeCO-k(PEG2PEG2gEC14)-Pen-N-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-E-N-3Pya-Sar-K(C14)-CONH2,
MeCO-Pen-N-T-7MeW—K(Ac)-Pen-AEF-2Nal-THP-K(Ac)—N-3Pya-Sar-K(PEG2PEG2gEC12)-CONH2,
MeCO-Pen-N-T-7MeW—K(Ac)-Pen-AEF-2Nal-THP-E-N-3Pya-Sar-K(PEG2PEG2gEDProC14)-CONH2,
MeCO-Pen-N-T-7MeW—K(Ac)-Pen-AEF-2Nal-THP-E-N-3Pya-Sar-K(PEG2PEG2C14)-CONH2,
MeCO-Pen-N-T-7MeW—K(Ac)-Pen-AEF-2Nal-THP-E-N-3Pya-Sar-K(GSGSGSGC14)-CONH2,
MeCO-Pen-N-T-7MeW—K(Ac)-Pen-AEF-2Nal-THP-E-N-3Pya-Sar-K(PEG2PEG2SP6C14)-CONH2,
MeCO-Pen-N-T-7MeW—K(Ac)-Pen-AEF-2Nal-THP-E-N-3Pya-Sar-K(PEG2C14)-CONH2,
MeCO-Pen-N-T-7MeW—K(Ac)-Pen-AEF-2Nal-THP-E-N-3Pya-Sar-K(PEG2PEG2gESarC14)-CONH2, and
MeCO-Pen-N-T-7MeW—K(Ac)-Pen-AEF-2Nal-THP-E-N-3Pya-Sar-K(PEG2PEG2gEProC14)-CONH2,
PEG2PEG2-gEC16OH-r-Pen-N-T-7MeW—K(Ac)-Pen-AEF-2Nal-THP-K(Ac)—N-3Pya-Sar-CONH2,
PEG2_Palm-Pen-N-T-7MeW—K(Ac)-Pen-AEF-2Nal-THP-E-N-3Pya-Sar-CONH2,
PEG2_Lauryl-Pen-N-T-7MeW—K(Ac)-Pen-AEF-2Nal-THP-E-N-3Pya-Sar-CONH2,
PEG4_Palm-Pen-N-T-7MeW—K(Ac)-Pen-AEF-2Nal-THP-E-N-3Pya-Sar-CONH2,
PEG4_Lauryl-Pen-N-T-7MeW—K(Ac)-Pen-AEF-2Nal-THP-E-N-3Pya-Sar-CONH2,
PEG4_Capryl-Pen-N-T-7MeW—K(Ac)-Pen-AEF-2Nal-THP-E-N-3Pya-Sar-CONH2,
PEG4_Hexyl-Pen-N-T-7MeW—K(Ac)-Pen-AEF-2Nal-THP-E-N-3Pya-Sar-CONH2,
C12gEPEG2PEG2CO-Pen-N-T-7MeW—K(PEG2PEG2gEC12)-Pen-AEF-2Nal-THP-E-N-3Pya-Sar-CONH2,
C14gEPEG2PEG2CO-Pen-N-T-7MeW—K(PEG2PEG2gEC14)-Pen-AEF-2Nal-THP-E-N-3Pya-Sar-CONH2,
C14gEPEG2PEG2CO-k(PEG2PEG2gEC14)-Pen-N-T-7MeW—K(Ac)-Pen-AEF-2Nal-THP-E-N-3Pya-Sar-CONH2,
C12gEPEG2PEG2CO-k(PEG2PEG2gEC12)-Pen-N-T-7MeW—K(Ac)-Pen-AEF-2Nal-THP-E-N-3Pya-Sar-CONH2,
C14gEPEG2PEG2CO-Pen-N-T-7MeW—K(Ac)-Pen-AEF-2Nal-THP-E-N-3Pya-Sar-K(PEG2PEG2gEC14)-CONH2,
C12gEPEG2PEG2CO-Pen-N-T-7MeW—K(Ac)-Pen-AEF-2Nal-THP-E-N-3Pya-Sar-CONH2,
C14gEPEG2PEG2CO-Pen-N-T-7MeW—K(Ac)-Pen-AEF-2Nal-THP-E-N-3Pya-Sar-CONH2,
C12gEPEG2PEG2CO-Pen-K(PEG2PEG2gEC12)-T-7MeW—K(Ac)-Pen-AEF-2Nal-THP-E-N-3Pya-Sar-CONH2,
C14gEPEG2PEG2CO-Pen-K(PEG2PEG2gEC14)-T-7MeW—K(Ac)-Pen-AEF-2Nal-THP-E-N-3Pya-Sar-CONH2,
C12gEPEG2PEG2CO-Pen-N-T-7MeW—K(Ac)-Pen-AEF-2Nal-THP-K(PEG2PEG2gEC12)-N-3Pya-Sar-CONH2,
C14gEPEG2PEG2CO-Pen-N-T-7MeW—K(Ac)-Pen-AEF-2Nal-THP-K(PEG2PEG2gEC14)-N-3Pya-Sar-CONH2,
C12gEPEG2PEG2CO-r-Pen-N-T-7MeW—K(Ac)-Pen-AEF-2Nal-THP-E-N-3Pya-Sar-CONH2,
C12gEPEG2PEG2CO-r-Pen-N-T-7MeW—K(Ac)-Pen-AEF-2Nal-THP-E-N-3Pya-Sar-K(PEG2PEG2gEC12)-CONH2,
C12gEPEG2PEG2CO-r-Pen-N-T-7MeW—K(Ac)-Pen-AEF-2Nal-THP-K(PEG2PEG2gEC12)-N-3Pya-Sar-CONH2,
C12gEPEG2PEG2CO-Pen-N-T-7MeW—K(Ac)-Pen-AEF(G)-2Nal-THP-E-N-3Pya-Sar-CONH2,
C12gEPEG2PEG2CO-r-Pen-N-T-7MeW—K(PEG2PEG2gEC12)-Pen-AEF-2Nal-THP-E-N-3Pya-Sar-CONH2,
C12gEPEG2PEG2CO-Pen-N-T-7MeW—K(Ac)-Pen-AEF-2Nal-THP-K(Ac)—N-3Pya-Sar-CONH2, and
C14gEPEG2PEG2CO-Pen-N-T-7MeW—K(Ac)-Pen-AEF-2Nal-TIP-K(Ac)—N-3Pya-Sar-CONH2.

In some embodiments, the present disclosure provides a peptide of Formula (I-A), comprising the amino acid sequence:

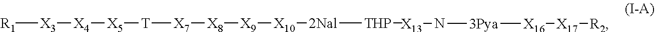

or a pharmaceutically acceptable salt thereof, wherein:
R₁ is MeCO, Z_{lipid}, Z_{peg}, succiniccarn, 5cpaCO, or AEEP-Z_{lipid};
X₃ is r, K—Z_{lipid}, or absent;
X₄ is Pen or Abu;
X₅ is N, N(NMe2), K(d), or K—Z_{lipid};
X₇ is 7MeW, W, or absent;
X₈ is K(Ac), K(d), K(NMeAc), Q, K—Z_{lipid}, Dab(NMecarn), Dab-Z_{peg};
X₉ is Pen or aMeC;
X₁₀ is AEF, TMAPF, AEF(d), APEG3F, TMAPF-Z_{lipid}, or AEF-Z_{lipid};
X₁₃ is E, K(Ac), K(d), K—Z_{peg}, K(NMeAc), Dab(NMecarn), E(OAll), K—Z_{lipid} or absent;
X₁₆ is Sar, NMeK(d), or NMeK—Z_{lipid};
X₁₇ is K—Z_{lipid} or absent; and
R₂ is CONH2, CONMe₂, CONH—Z_{peg}, or CO—Z_{lipid};
wherein the peptide is cyclized via a disulfide or thioether linkage between the residues at X₄ and X₉; and
wherein the polypeptide comprises at least one lipid-containing amino acid.

In some embodiments, the present disclosure provides a peptide of Formula (I-B), comprising the amino acid sequence:

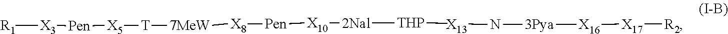

wherein:
R₁ is MeCO, Z_{lipid}, Z_{peg}, succiniccarn, or 5cpaCO;
X₃ is r, K—Z_{lipid}, Dab-Z_{lipid}, NMeK—Z_{lipid}, or absent;
X₅ is N, N(NMe₂), or K—Z_{lipid};
X₈ is K(Ac), K(d), K(NMeAc), K—Z_{lipid}, Dab-Z_{peg};
X₁₀ is AEF, TMAPF, APEG3F, or AEF-Z_{lipid};
X₁₃ is E, K(Ac), K(d), K—Z_{peg}, K(NMeAc), or K—Z_{lipid};
X₁₆ is Sar, or NMeK—Z_{lipid};
X₁₇ is K—Z_{lipid} or absent; and
R₂ is CONH2, CONMe₂, or CONH—Z_{peg}.
wherein the peptide is cyclized via a disulfide or thioether linkage between the residues at X₄ and X₉; and
wherein the polypeptide comprises at least one lipid-containing amino acid.

In some embodiments, the present disclosure provides a peptide of Formula (I-B), comprising the amino acid sequence:

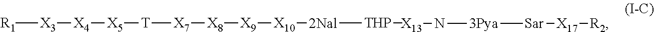

wherein:
R₁ is MeCO, Z_{lipid}, succiniccarn, 5cpaCO, or AEEP-Z_{lipid};
X₃ is R, K—Z_{lipid}, Dab-Z_{lipid}, NMeK—Z_{lipid}, or absent;
X₄ is 4AminoPro, Abu, aG, aMeC, C, Dap, Pen, Pen(oXyl), Pen(mXyl), Pen(pXyl), or Pra;
X₅ is N, N(NMe₂), K(d), K—Z_{lipid}, Dab-Z_{lipid}, or NMeK—Z_{lipid};
X₇ is 7MeW, W, or absent;
X₈ is K(Ac), K(d), K(NMeAc), K—Z_{lipid}, Dab(NMecarn);
X₉ is aMeC, aG, C, D, E, hE, Pen, Dap, or Dap(N3);
X₁₀ is AEF, TMAPF, TMAPF-Z_{lipid}, or AEF-Z_{lipid};
X₁₃ is E, K(Ac), K(d), K—Z_{peg}, K(NMeAc), or K—Z_{lipid};
X₁₇ is absent; and
R₂ is CONH2, CONMe₂, or CONH—Z_{peg}.
wherein the peptide is cyclized via a disulfide or thioether linkage between the residues at X₄ and X₉; and
wherein the polypeptide comprises at least one lipid-containing amino acid.

In some embodiments, R₁ is MeCO, Z_{peg} (i.e., a polyethylene glycol chain), Z_{lipid}(i.e., a lipophilic substituent), succiniccarn, 5cpaCO, or AEEP-Z_{lipid}. In some embodiments, R₁ is MeCO, Z_{peg}, Z_{lipid}, succiniccarn, or 5cpaCO. In some embodiments, R₁ is MeCO, Z_{peg}, Z_{lipid}, or succiniccarn. In some embodiments, R₁ is MeCO, Z_{peg} or Z_{lipid}. In some embodiments, R₁ is MeCO, cPEG3aCO, or Z_{lipid} In some embodiments, R₁ is MeCO or Z_{peg}. In some embodiments, R₁ is MeCO or cPEG3aCO.

In some embodiments, R₁ is an alkyl or polyethylene glycol chain. In some embodiments, R₁ is 5cpaCO. In some embodiments, R₁ is AEEP-Z_{lipid}. In some embodiments, R₁ is succiniccarn. In some embodiments, R₁ is MeCO. In some embodiments, R₁ is Z_{peg}, i.e., a polyethylene glycol chain. In some embodiments, R₁ is a polyethylene glycol chain terminating in an ammonium, biotin, or FITC. In some embodiments, R₁ is a polyethylene glycol chain terminating in an ammonium group. In some embodiments, R₁ is cPEG3aCO or cPEG5aCO. In some embodiments, R₁ is cPEG3aCO.

In some embodiments, R₁ is Z_{lipid}, i.e., a lipophilic substituent. In some embodiments, R₁ is a lipophilic substituent terminating in a carboxylic acid or phenoxy group. In some embodiments, R₁ is a lipophilic substituent terminating in a carboxylic acid group. In some embodiments, R₁ is HOC18gEPEG2PEG2CO.

In some embodiments, X₃ is K—Z_{lipid}, Dab-Z_{lipid}, NMeK—Z_{lipid}, R, or absent, wherein the K—Z_{lipid}, Dab-Z_{lipid}, NMeK—Z_{lipid}, and R are L amino acids. In some embodiments, X₃ is k-Z_{lipid}, dDab-Z_{lipid}, NMek-Z_{lipid}, r, or absent.

In some embodiments, X₃ is K—Z_{lipid}, Dab-Z_{lipid}, NMeK—Z_{lipid}, R, or absent. In some embodiments, X₃ is K—Z_{lipid}, Dab-Z_{lipid}, R, or absent. In some embodiments, X₃ is K—Z_{lipid}, NMeK—Z_{lipid}, R, or absent. In some embodiments, X₃ is K—Z_{lipid}, R, or absent.

In some embodiments, X₃ is K—Z_{lipid}, Dab-Z_{lipid}, NMeK—Z_{lipid}, R or absent, wherein when X₃ is K—Z_{lipid}, Dab-Z_{lipid}, or NMeK—Z_{lipid}, then the lipophilic substituent is selected from PEG12gEC18OH, PEG12gEC20OH, PEG2PEG2gEC18OH, and PEG2PEG2gEC20OH. In some embodiments, X₃ is K—Z_{lipid}, Dab-Z_{lipid}, R or absent, wherein when X₃ is K—Z_{lipid} or Dab-Z _{lipid}, then the lipophilic substituent is selected from PEG12gEC18OH, PEG12gEC20OH, PEG2PEG2gEC18OH, and PEG2PEG2gEC20OH. In some embodiments, X₃ is K—Z_{lipid}, NMeK—Z_{lipid}, R or absent, wherein when X₃ is K—Z_{lipid}, or NMeK—Z_{lipid}, then the lipophilic substituent is selected from PEG12gEC18OH, PEG12gEC20OH, PEG2PEG2gEC18OH, and PEG2PEG2gEC20OH.

In some embodiments, X₃ is K—Z_{lipid}, R or absent, wherein when X₃ is K—Z_{lipid}, then the lipophilic substituent is selected from PEG12gEC18OH, PEG12gEC20OH, PEG2PEG2gEC18OH, and PEG2PEG2gEC20OH. In some embodiments, X₃ is K—Z_{lipid}, R or absent, wherein when X₃ is K—Z_{lipid}, then the lipophilic substituent is selected from PEG12gEC18OH, PEG12gEC20OH, and PEG2PEG2gEC18OH. In some embodiments, X₃ is K—Z_{lipid}, R or absent, wherein when X₃ is K—Z_{lipid}, then the lipophilic substituent is selected from PEG12gEC18OH and PEG2PEG2gEC18OH. In some embodiments, X₃ is K—Z_{lipid}, R or absent, wherein when X₃ is K—Z_{lipid}, then the lipophilic substituent is selected from PEG12gEC20OH and PEG2PEG2gEC18OH. In some embodiments, X₃ is K—Z_{lipid}, R or absent, wherein when X₃ is K—Z_{lipid}, then the lipophilic substituent is PEG2PEG2gEC18OH.

In some embodiments, X₃ is K—Z_{lipid}, Dab-Z_{lipid}, NMeK—Z_{lipid}, wherein lipophilic substituent is selected from PEG12gEC18OH, PEG12gEC20OH, PEG2PEG2gEC18OH, and PEG2PEG2gEC20OH. In some embodiments, X₃ is K—Z_{lipid}, Dab-Z_{lipid}, NMeK—Z_{lipid}, wherein lipophilic substituent is selected from PEG12gEC18OH, PEG12gEC20OH, and PEG2PEG2gEC18OH. In some embodiments, X₃ is K—Z_{lipid}, Dab-Z_{lipid}, NMeK—Z_{lipid}, wherein lipophilic substituent is PEG12gEC18OH or PEG12gEC20OH. In some embodiments, X₃ is K—Z_{lipid}, Dab-Z_{lipid}, NMeK—Z_{lipid}, wherein lipophilic substituent is PEG12gEC18OH or PEG2PEG2gEC18OH. In some embodiments, X₃ is K—Z_{lipid}, Dab-Z_{lipid}, NMeK—Z_{lipid}, wherein lipophilic substituent is PEG12gEC20OH or PEG2PEG2gEC18OH.

In some embodiments, X₃ is K—Z_{lipid} or NMeK—Z_{lipid}, wherein lipophilic substituent is selected from PEG12gEC18OH, PEG12gEC20OH, PEG2PEG2gEC18OH, and PEG2PEG2gEC20OH. In some embodiments, X₃ is K—Z_{lipid} or Dab-Z_{lipid}, wherein lipophilic substituent is selected from PEG12gEC18OH, PEG12gEC20OH, PEG2PEG2gEC18OH, and PEG2PEG2gEC20OH. In some embodiments, X₃ is K—Z_{lipid} wherein lipophilic substituent is selected from PEG12gEC18OH, PEG12gEC20OH, PEG2PEG2gEC18OH, and PEG2PEG2gEC20OH. In some embodiments, X₃ is K—Z_{lipid} wherein lipophilic substituent is selected from PEG12gEC18OH, PEG12gEC20OH, and PEG2PEG2gEC18OH. In some embodiments, X₃ is K—Z_{lipid}, wherein lipophilic substituent is PEG12gEC18OH or PEG2PEG2gEC18OH. In some embodiments, X₃ is K—Z_{lipid}, wherein lipophilic substituent is PEG12gEC20OH or PEG2PEG2gEC18OH. In some embodiments, X₃ is K—Z_{lipid}, wherein lipophilic substituent is PEG2PEG2gEC18OH.

In some embodiments, X₃ is K—Z_{lipid}, NMeK—Z_{lipid}, or Dab-Z_{lipid}. In some embodiments, X₃ is k-Z_{lipid}, NMek-Z_{lipid}, or dDab-Z_{lipid}. In some embodiments, X₃ is K—Z_{lipid}. In some embodiments, X₃ is NMeK—Z_{lipid}. In some embodiments, X₃ is Dab-Z_{lipid}. In some embodiments, X₃ is NMek-Z_{lipid}. In some embodiments, X₃ is dDab-Z_{lipid}.

In some embodiments, X₃ is k-Z_{lipid}, r, or absent. In some embodiments, X₃ is k-Z_{lipid}, wherein lipophilic substituent is PEG2PEG2gEC18OH. In some embodiments, X₃ is k-Z_{lipid}. In some embodiments, X₃ is R or absent. In some embodiments, X₃ is R. In some embodiments, X₃ is r or absent. In some embodiments, X₃ is absent. In some embodiments, X₃ is r.

In some embodiments, X₄ is 4AminoPro, Abu, aG, aMeC, C, Dap, Pen, Pen(oXyl), Pen(mXyl), Pen(pXyl), or Pra. In some embodiments, X₄ is 4AminoPro, Abu, aG, aMeC, C, Dap, Pen, Pen(oXyl), Pen(mXyl), Pen(pXyl), or Pra, each of which is an L amino acid. In some embodiments, X₄ is 4Amino-d-Pro, dAbu, d-aG, aMe-d-C, c, dDap, dPen, dPen(oXyl), dPen(mXyl), dPen(pXyl), or dPra.

In some embodiments, X₄ is 4AminoPro, aG, Dap, Pen(oXyl), Pen(mXyl), Pen(pXyl), or Pra. In some embodiments, X₄ is Abu, aMeC, C, Pen, Pen(oXyl), Pen(mXyl), or Pen(pXyl). In some embodiments, X₄ is Abu, aMeC, C, or Pen. In some embodiments, X₄ is Abu, C, or Pen. In some embodiments, X₄ is Abu or Pen.

In some embodiments, X₄ is 4AminoPro. In some embodiments, X₄ is 4RAminoPro. In some embodiments, X₄ is 4SAminoPro. In some embodiments, X₄ is Abu. In some embodiments, X₄ is aG. In some embodiments, X₄ is aMeC. In some embodiments, X₄ is C. In some embodiments, X₄ is Dap. In some embodiments, X₄ is Pen. In some embodiments, X₄ is Pen(oXyl). In some embodiments, X₄ is Pen(mXyl). In some embodiments, X₄ is Pen(pXyl). In some embodiments, X₄ is Pra.

In some embodiments, X₄ is 4Amino-d-Pro. In some embodiments, X₄ is 4RAmino-d-Pro. In some embodiments, X₄ is 4SAmino-d-Pro. In some embodiments, X₄ is dAbu. In some embodiments, X₄ is d-aG. In some embodiments, X₄ is aMe-d-C. In some embodiments, X₄ is c. In some embodiments, X₄ is dDap. In some embodiments, X₄ is dPen. In some embodiments, X₄ is dPen(oXyl). In some embodiments, X₄ is dPen(mXyl). In some embodiments, X₄ is dPen(pXyl). In some embodiments, X₄ is dPra.

In some embodiments, X₅ is K(d), K—Z_{lipid}, NMeK—Z_{lipid}, Dab-Z_{lipid}, N, or N(NMe2), each of which is an L amino acid. In some embodiments, X₅ is k(d), k-Z_{lipid}, NMek-Z_{lipid}, dDab-Z_{lipid}, n, or n(NMe2).

In some embodiments, X₅ is K(d), K—Z_{lipid}, NMeK—Z_{lipid}, Dab-Z_{lipid}, N, or N(NMe2). In some embodiments, X₅ is K(d), K—Z_{lipid}, NMeK—Z_{lipid}, N, or N(NMe2). In some embodiments, X₅ is K(d), K—Z_{lipid}, Dab-Z_{lipid}, N, or N(NMe2). In some embodiments, X₅ is K(d), K—Z_{lipid}, N, or N(NMe2). In some embodiments, X₅ is K—Z_{lipid}, N, or N(NMe2).

In some embodiments, X₅ is K(d), K—Z_{lipid}, Dab-Z_{lipid}, NMeK—Z_{lipid}, N or N(NMe2), wherein when X₅ is K—Z_{lipid}, Dab-Z_{lipid}, or NMeK—Z_{lipid}, then the lipophilic substituent is selected from PEG12gEC18OH, PEG12gEC20OH, PEG2PEG2gEC18OH, and PEG2PEG2gEC20OH. In some embodiments, X₅ is K(d), K—Z_{lipid}, NMeK—Z_{lipid}, N or N(NMe2), wherein when X₅ is K—Z_{lipid} or NMeK—Z_{lipid}, then the lipophilic substituent is selected from PEG12gEC18OH, PEG12gEC20OH, PEG2PEG2gEC18OH, and PEG2PEG2gEC20OH. In some embodiments, X₅ is K(d), K—Z_{lipid}, Dab-Z_{lipid}, N or N(NMe2), wherein when X₅ is K—Z_{lipid} or Dab-Z_{lipid}, then the lipophilic substituent is selected from PEG12gEC18OH, PEG12gEC20OH, PEG2PEG2gEC18OH, and PEG2PEG2gEC20OH.

In some embodiments, X₅ is K(d), K—Z_{lipid}, N or N(NMe2), wherein when X₅ is K—Z_{lipid}, then the lipophilic substituent is selected from PEG12gEC18OH, PEG12gEC20OH, PEG2PEG2gEC18OH, and PEG2PEG2gEC20OH. In some embodiments, X₅ is K(d), K—Z_{lipid}, N or N(NMe2), wherein when X₅ is K—Z_{lipid}, then the lipophilic substituent is selected from PEG12gEC18OH, PEG12gEC20OH, and PEG2PEG2gEC18OH. In some embodiments, X₅ is K(d), K—Z_{lipid}, N or N(NMe2), wherein when X₅ is K—Z_{lipid}, then the lipophilic substituent is selected from PEG12gEC18OH and PEG2PEG2gEC18OH. In some embodiments, X₅ is K(d), K—Z_{lipid}, N or N(NMe2), wherein when X₅ is K—Z_{lipid}, then the lipophilic substituent is selected from PEG12gEC20OH and PEG2PEG2gEC18OH. In some embodiments, X₅ is K(d), K—Z_{lipid}, N or N(NMe2), wherein when X₅ is K—Z_{lipid}, then the lipophilic substituent is PEG2PEG2gEC18OH.

In some embodiments, X₅ is K—Z_{lipid}, Dab-Z_{lipid}, NMeK—Z_{lipid}, wherein lipophilic substituent is selected from PEG12gEC18OH, PEG12gEC20OH, PEG2PEG2gEC18OH, and PEG2PEG2gEC20OH. In some embodiments, X₅ is K—Z_{lipid}, Dab-Z_{lipid}, NMeK—Z_{lipid}, wherein lipophilic substituent is selected from PEG12gEC18OH, PEG12gEC20OH, and PEG2PEG2gEC18OH. In some embodiments, X₅ is K—Z_{lipid}, Dab-Z_{lipid}, NMeK—Z_{lipid}, wherein lipophilic substituent is PEG12gEC18OH or PEG12gEC20OH. In some embodiments, X₅ is K—Z_{lipid}, Dab-Z_{lipid}, NMeK—Z_{lipid}, wherein lipophilic substituent is PEG12gEC18OH or PEG2PEG2gEC18OH. In some embodiments, X₅ is K—Z_{lipid}, Dab-Z_{lipid}, NMeK—Z_{lipid}, wherein lipophilic substituent is PEG12gEC20OH or PEG2PEG2gEC18OH.

In some embodiments, X₅ is K—Z_{lipid} wherein lipophilic substituent is selected from PEG12gEC18OH, PEG12gEC20OH, PEG2PEG2gEC18OH, and PEG2PEG2gEC20OH. In some embodiments, X₅ is K—Z_{lipid} wherein lipophilic substituent is selected from PEG12gEC18OH, PEG12gEC20OH, and PEG2PEG2gEC18OH. In some embodiments, X₅ is K—Z_{lipid}, wherein lipophilic substituent is PEG12gEC18OH or PEG2PEG2gEC18OH. In some embodiments, X₅ is K—Z_{lipid}, wherein lipophilic substituent is PEG12gEC20OH or PEG2PEG2gEC18OH. In some embodiments, X₅ is K—Z_{lipid}, wherein lipophilic substituent is PEG2PEG2gEC18OH.

In some embodiments, X₅ is K—Z_{lipid}, NMeK—Z_{lipid}, or Dab-Z_{lipid}. In some embodiments, X₅ is k-Z_{lipid}, NMek-Z_{lipid}, or dDab-Z_{lipid}. In some embodiments, X₅ is K—Z_{lipid}. In some embodiments, X₅ is NMeK—Z_{lipid}. In some embodiments, X₅ is Dab-Z_{lipid}. In some embodiments, X₅ is NMek-Z_{lipid}. In some embodiments, X₅ is dDab-Z_{lipid}.

In some embodiments, X₅ is k(d) k-Z_{lipid}, n, or n(NMe2). In some embodiments, X₅ is K(d). In some embodiments, X₅ is k(d). In some embodiments, X₅ is k-Z_{lipid}. In some embodiments, X₅ is N or N(NMe2). In some embodiments, X₅ is N. In some embodiments, X₅ is N(NMe2). In some embodiments, X₅ is n or n(NMe2). In some embodiments, X₅ is n. In some embodiments, X₅ is n.

In some embodiments, X₆ is T, K—Z_{lipid}, NMeK—Z_{lipid}, or Dab-Z_{lipid}, each of which is an L amino acid. In some embodiments, X₆ is t, k-Z_{lipid}, NMek-Z_{lipid}, or dDab-Z_{lipid}.

In some embodiments, X₆ is T, K—Z_{lipid}, NMeK—Z_{lipid}, or Dab-Z_{lipid}. In some embodiments, X₆ is T, K—Z_{lipid} or NMeK—Z_{lipid}. In some embodiments, X₆ is T, K—Z_{lipid}, or Dab-Z_{lipid}. In some embodiments, X₆ is T or K—Z_{lipid}.

In some embodiments, X₆ is T, K—Z_{lipid}, NMeK—Z_{lipid}, or Dab-Z_{lipid}, wherein when X₆ is K—Z_{lipid}, Dab-Z_{lipid}, or NMeK—Z_{lipid}, then the lipophilic substituent is selected from PEG12gEC18OH, PEG12gEC20OH, PEG2PEG2gEC18OH, and PEG2PEG2gEC20OH. In some embodiments, X₆ is T, K—Z_{lipid} or NMeK—Z_{lipid}, wherein when X₆ is K—Z_{lipid} or NMeK—Z_{lipid}, then the lipophilic substituent is selected from PEG12gEC18OH, PEG12gEC20OH, PEG2PEG2gEC18OH. In some embodiments, X₆ is T, K—Z_{lipid}, or Dab-Z_{lipid}, wherein when X₆ is K—Z_{lipid} or Dab-Z_{lipid}, then the lipophilic substituent is selected from PEG12gEC18OH, PEG12gEC20OH, and PEG2PEG2gEC18OH. In some embodiments, X₆ is T or K—Z_{lipid}, wherein when X₆ is K—Z_{lipid}, then the lipophilic substituent is selected from PEG12gEC18OH, PEG12gEC20OH, and PEG2PEG2gEC18OH.

In some embodiments, X₆ is K—Z_{lipid}, Dab-Z_{lipid}, NMeK—Z_{lipid}, wherein lipophilic substituent is selected from PEG12gEC18OH, PEG12gEC20OH, PEG2PEG2gEC18OH, and PEG2PEG2gEC20OH. In some embodiments, X₆ is K—Z_{lipid} or NMeK—Z_{lipid}, wherein lipophilic substituent is selected from PEG12gEC18OH, PEG12gEC20OH, PEG2PEG2gEC18OH, and PEG2PEG2gEC20OH. In some embodiments, X₆ is K—Z_{lipid} or Dab-Z_{lipid}, wherein lipophilic substituent is selected from PEG12gEC18OH, PEG12gEC20OH, PEG2PEG2gEC18OH, and PEG2PEG2gEC20OH. In some embodiments, X₆ is K—Z_{lipid} wherein lipophilic substituent is selected from PEG12gEC18OH, PEG12gEC20OH, PEG2PEG2gEC18OH, and PEG2PEG2gEC20OH.

In some embodiments, X₆ is K—Z_{lipid}, NMeK—Z_{lipid}, or Dab-Z_{lipid}. In some embodiments, X₆ is k-Z_{lipid}, NMek-Z_{lipid}, or dDab-Z_{lipid}. In some embodiments, X₆ is T or K—Z_{lipid}. In some embodiments, X₆ is K—Z_{lipid}. In some embodiments, X₆ is NMeK—Z_{lipid}. In some embodiments, X₆ is Dab-Z_{lipid}. In some embodiments, X₆ is T. In some embodiments, X₆ is NMek-Z_{lipid}. In some embodiments, X₆ is dDab-Z_{lipid}. In some embodiments, X₆ is k-Z_{lipid}. In some embodiments, X₆ is t.

In some embodiments, X₇ is 7MeW, W, or absent. In some embodiments, X₇ is 7MeW, W, or absent, wherein the 7MeW and W are L amino acids. In some embodiments, X₇ is 7Mew, w, or absent.

In some embodiments, X₇ is 7MeW or W. In some embodiments, X₇ is 7MeW. In some embodiments, X₇ is W. In some embodiments, X₇ is 7Mew. In some embodiments, X₇ is w. In some embodiments, X₇ is absent.

In some embodiments, X₈ is K(Ac), K(d), K(NMeAc), Q, K—Z_{lipid}, K—Z_{peg}, NMeK—Z_{lipid}, Dab-Z_{lipid}, Dab(NMecarn), or Dab-Z_{peg}. In some embodiments, X₈ is K(Ac), K(d), K(NMeAc), Q, K—Z_{lipid}, K—Z_{peg}, NMeK—Z_{lipid}, Dab-Z_{lipid}, Dab(NMecarn), or Dab-Z_{peg}, each of which is an L-amino acid. In some embodiments, X₈ is k(Ac), k(d), k(NMeAc), q, k-Z_{lipid}, k-Z_{peg}, NMek-Z_{lipid}, dDab-Z_{lipid}, dDab(NMecarn), or dDab-Z_{peg}.

In some embodiments, X₈ is K(Ac), K(d), K(NMeAc), Q, K—Z_{lipid}, K—Z_{peg}, NMeK—Z_{lipid}, or Dab(NMecarn). In some embodiments, X₈ is K(Ac), K(d), K(NMeAc), K—Z_{lipid}, K—Z_{peg}, NMeK—Z_{lipid}, or Dab(NMecarn). In some embodiments, X₈ is K(Ac), K(d), K(NMeAc), K—Z_{lipid}, or K—Z_{peg}. In some embodiments, X₈ is K(Ac), K(NMeAc), or K—Z_{lipid}. In some embodiments, X₈ is K(Ac) or K(NMeAc).

In some embodiments, X₈ is K(Ac), K(d), K(NMeAc), Q, K—Z_{lipid}, K—Z_{peg}, NMeK—Z_{lipid}, or Dab-Z_{lipid}, Dab(NMecarn), or Dab-Z_{peg}, wherein when X₈ is K—Z_{lipid}, Dab-Z_{lipid}, or NMeK—Z_{lipid}, then the lipophilic substituent is selected from PEG12gEC18OH, PEG2PEG2C18OH, PEG2PEG2gEC18OH, and PEG2PEG2gEC20OH. In some embodiments, X₈ is K(Ac), K(d), K(NMeAc), Q, K—Z_{lipid}, K—Z_{peg}, NMeK—Z_{lipid}, Or Dab-Z_{lipid}, Dab(NMecarn), or Dab-Z_{peg}, wherein when X₈ is K—Z_{lipid}, Dab-Z_{lipid}, or NMeK—Z_{lipid}, then the lipophilic substituent is selected from PEG12gEC18OH, PEG2PEG2C18OH, and PEG2PEG2gEC18OH. In some embodiments, X₈ is K(Ac), K(d), K(NMeAc), Q, K—Z_{lipid}, K—Z_{peg}, NMeK—Z_{lipid}, or Dab-Z_{lipid}, Dab(NMecarn), or Dab-Z_{peg}, wherein when X₈ is k-Z_{lipid}, dDab-Z_{lipid}, or NMek-Z_{lipid}, then the lipophilic substituent is selected from PEG12gEC18OH or PEG2PEG2gEC18OH. In some embodiments, X₈ is K(Ac), K(d), K(NMeAc), Q, K—Z_{lipid}, K—Z_{peg}, NMeK—Z_{lipid}, or Dab-Z_{lipid}, Dab(NMecarn), or Dab-Z_{peg}, wherein when X₈ is k-Z_{lipid}, dDab-Z_{lipid}, or NMek-Z_{lipid}, then the lipophilic substituent is selected from PEG2PEG2C18OH or PEG2PEG2gEC18OH. In some embodiments, X₈ is K(Ac), K(d), K(NMeAc), Q, K—Z_{lipid}, K—Z_{peg}, NMeK—Z_{lipid}, or Dab-Z_{lipid}, Dab(NMecarn), or Dab-Z_{peg}, wherein when X₈ is k-Z_{lipid}, dDab-Z_{lipid}, or NMek-Z_{lipid}, then the lipophilic substituent is PEG2PEG2gEC18OH.

In some embodiments, X₈ is K(Ac), K(d), K(NMeAc), K—Z_{lipid}, or Dab(NMecarn), wherein lipophilic substituent is selected from PEG12gEC18OH, PEG2PEG2gEC18OH, PEG2PEG2gEC18OH, and PEG2PEG2gEC20OH. In some embodiments, X₈ is K(Ac), K(d), K(NMeAc), K—Z_{lipid}, or Dab(NMecarn), wherein lipophilic substituent is selected from PEG12gEC18OH, PEG2PEG2gEC18OH, and PEG2PEG2gEC18OH. In some embodiments, X₈ is K(Ac), K(d), K(NMeAc), K—Z_{lipid}, or Dab(NMecarn), wherein lipophilic substituent is selected from PEG12gEC18OH, and PEG2PEG2gEC18OH. In some embodiments, X₈ is K(Ac), K(d), K(NMeAc), K—Z_{lipid}, or Dab(NMecarn), wherein lipophilic substituent is selected from PEG2PEG2C18OH, and PEG2PEG2gEC18OH. In some embodiments, X₈ is K(Ac), K(d), K(NMeAc), K—Z_{lipid}, or Dab(NMecarn), wherein lipophilic substituent is PEG2PEG2gEC18OH.

I In some embodiments, X₈ is K(Ac). In some embodiments, X₈ is Dab(NMecarn). In some embodiments, X₈ is Dab-Z_{peg}. In some embodiments, X₈ is Dab-Z_{lipid}. In some embodiments, X₈ is K(d). In some embodiments, X₈ is K(NMeAc). In some embodiments, X₈ is K—Z_{peg}. In some embodiments, X₈ is K—Z_{lipid}. In some embodiments, X₈ is K—Z_{lipid} wherein lipophilic substituent is selected from PEG12gEC18OH, PEG2PEG2C18OH, PEG2PEG2gEC18OH, and PEG2PEG2gEC20OH. In some embodiments, X₈ is NMeK—Z_{lipid}. In some embodiments, X₈ is Q.

In some embodiments, X₈ is k(Ac). In some embodiments, X₈ is dDab(NMecarn). In some embodiments, X₈ is dDab-Z_{peg}. In some embodiments, X₈ is dDab-Z_{lipid}. In some embodiments, X₈ is k(d). In some embodiments, X₈ is k(NMeAc). In some embodiments, X₈ is k-Z_{peg}. In some embodiments, X₈ is k-Z_{lipid}. In some embodiments, X₈ is k-Z_{lipid} wherein lipophilic substituent is selected from PEG12gEC18OH, PEG2PEG2C18OH, PEG2PEG2gEC18OH, and PEG2PEG2gEC20OH. In some embodiments, X₈ is NMek-Z_{lipid}. In some embodiments, X₈ is q.

In some embodiments, X₉ is aMeC, aG, C, D, E, hE, Pen, or Dap(N3). In some embodiments, X₉ is aMeC, aG, C, D, E, hE, Pen, or Dap(N3), each of which is an L-amino acid. In some embodiments, X₉ is aMe-d-C, d-aG, c, d, e, he, dPen, or dDap(N3).

In some embodiments, X₉ is aG, D, E, hE, or Dap(N3). In some embodiments, X₉ is aMeC, C, or Pen. In some embodiments, X₉ is aMeC or Pen.

In some embodiments, X₉ is aMeC. In some embodiments, X₉ is aG. In some embodiments, X₉ is C. In some embodiments, X₉ is D. In some embodiments, X₉ is E. In some embodiments, X₉ is hE. In some embodiments, X₉ is Pen. In some embodiments, X₉ is Dap(N3).

In some embodiments, X₉ is aMe-d-C. In some embodiments, X₉ is d-aG. In some embodiments, X₉ is c. In some embodiments, X₉ is d. In some embodiments, X₉ is e. In some embodiments, X₉ is he. In some embodiments, X₉ is dPen. In some embodiments, X₉ is dDap(N3).

In some embodiments, X₁₀ is AEF, TMAPF, AEF(d), TMAPF-Z_{lipid}, APEG3F, or AEF-Z_{lipid}. In some embodiments, X₁₀ is AEF, TMAPF, AEF(d), TMAPF-Z_{lipid}, APEG3F, or AEF-Z_{lipid}, each of which is an L-amino acid. In some embodiments, X₁₀ is dAEF, dTMAPF, dAEF(d), dTMAPF-Z_{lipid}, dAPEG3F, or dAEF-Z_{lipid}.

In some embodiments, X₁₀ is AEF, TMAPF, AEF(d), APEG3F, or AEF-Z_{lipid}. In some embodiments, X₁₀ is AEF, TMAPF, APEG3F, or AEF-Z_{lipid}. In some embodiments, X₁₀ is AEF, TMAPF or APEG3F. In some embodiments, X₁₀ is AEF or TMAPF.

In some embodiments, X₁₀ is AEF, TMAPF, TMAPF-Z_{lipid}, AEF(d), APEG3F, or AEF-Z_{lipid}, wherein when X₁₀ is TMAPF-Z_{lipid} or AEF-Z_{lipid}, then the lipophilic substituent is selected from SP6PEG12gEC18OH, SP6PEG12gEC20OH, SP6PEG2PEG2gEC18OH, SP6PEG2PEG2gEC20OH, SP6PEG6gEC18OH, SP6PEG6gEC20OH, and PEG2PEG2gEC18OH. In some embodiments, X₁₀ is AEF, TMAPF, TMAPF-Z_{lipid}, AEF(d), APEG3F, or AEF-Z_{lipid}, wherein when X₁₀ is TMAPF-Z_{lipid} or AEF-Z_{lipid}, then the lipophilic substituent is selected from SP6PEG2PEG2gEC18OH, SP6PEG2PEG2gEC20OH, SP6PEG6gEC18OH, SP6PEG6gEC20OH, and PEG2PEG2gEC18OH. In some embodiments, X₁₀ is AEF, TMAPF, TMAPF-Z_{lipid}, AEF(d), APEG3F, or AEF-Z_{lipid}, wherein when X₁₀ is TMAPF-Z_{lipid} or AEF-Z_{lipid}, then the lipophilic substituent is selected from SP6PEG12gEC18OH, SP6PEG12gEC20OH, SP6PEG2PEG2gEC18OH, SP6PEG2PEG2gEC20OH, and PEG2PEG2gEC18OH. In some embodiments, X₁₀ is AEF, TMAPF, TMAPF-Z_{lipid}, AEF(d), APEG3F, or AEF-Z_{lipid}, wherein when X₁₀ is TMAPF-Z_{lipid} or AEF-Z_{lipid}, then the lipophilic substituent is selected from SP6PEG12gEC18OH, SP6PEG12gEC20OH, SP6PEG6gEC18OH, SP6PEG6gEC20OH, and PEG2PEG2gEC18OH.

In some embodiments, X₁₀ is AEF, TMAPF, TMAPF-Z_{lipid}, AEF(d), APEG3F, or AEF-Z_{lipid}, wherein when X₁₀ is TMAPF-Z_{lipid} or AEF-Z_{lipid}, then the lipophilic substituent is selected from SP6PEG12gEC18OH, SP6PEG12gEC20OH, and PEG2PEG2gEC18OH. In some embodiments, X₁₀ is AEF, TMAPF, TMAPF-Z_{lipid}, AEF(d), APEG3F, or AEF-Z_{lipid}, wherein when X₁₀ is TMAPF-Z_{lipid} or AEF-Z_{lipid}, the lipophilic substituent is selected from SP6PEG2PEG2gEC18OH, SP6PEG2PEG2gEC20OH, and PEG2PEG2gEC18OH. In some embodiments, X₁₀ is AEF, TMAPF, TMAPF-Z_{lipid}, AEF(d), APEG3F, or AEF-Z_{lipid}, wherein when X₁₀ is TMAPF-Z_{lipid} or AEF-Z_{lipid}, the lipophilic substituent is selected from SP6PEG6gEC18OH, SP6PEG6gEC20OH, and PEG2PEG2gEC18OH. In some embodiments, X₁₀ is AEF, TMAPF, TMAPF-Z_{lipid}, AEF(d), APEG3F, or AEF-Z_{lipid}, wherein when X₁₀ is TMAPF-Z_{lipid} or AEF-Z_{lipid}, the lipophilic substituent is PEG2PEG2gEC18OH.

In some embodiments, X₁₀ is AEF. In some embodiments, X₁₀ is TMAPF. In some embodiments, X₁₀ is TMAPF-Z_{lipid}. In some embodiments, X₁₀ is TMAPF-Z_{lipid}, wherein the lipophilic substituent is PEG2PEG2gEC18OH. In some embodiments, X₁₀ is AEF(d). In some embodiments, X₁₀ is APEG3F. In some embodiments, X₁₀ is AEF-Z_{lipid}. In some embodiments, X₁₀ is AEF-Z_{lipid}, wherein the lipophilic substituent is selected from SP6PEG12gEC18OH, SP6PEG12gEC20OH, SP6PEG2PEG2gEC18OH, SP6PEG2PEG2gEC20OH, SP6PEG6gEC18OH, SP6PEG6gEC20OH, and PEG2PEG2gEC18OH. In some embodiments, X₁₀ is AEF-Z_{lipid}, wherein the lipophilic substituent is PEG2PEG2gEC18OH.

In some embodiments, X₁₀ is dAEF. In some embodiments, X₁₀ is dTMAPF. In some embodiments, X₁₀ is dTMAPF-Z_{lipid}. In some embodiments, X₁₀ is dTMAPF-Z_{lipid}, wherein the lipophilic substituent is PEG2PEG2gEC18OH. In some embodiments, X₁₀ is dAEF(d). In some embodiments, X₁₀ is dAPEG3F. In some embodiments, X₁₀ is dAEF-Z_{lipid}. In some embodiments, X₁₀ is dAEF-Z_{lipid}, wherein the lipophilic substituent is selected from SP6PEG12gEC18OH, SP6PEG12gEC20OH, SP6PEG2PEG2gEC18OH, SP6PEG2PEG2gEC20OH, SP6PEG6gEC18OH, SP6PEG6gEC20OH, and PEG2PEG2gEC18OH. In some embodiments, X₁₀ is dAEF-Z_{lipid}, wherein the lipophilic substituent is PEG2PEG2gEC18OH.

In some embodiments, X₁₁ is 2Nal. In some embodiments, X₁₁ is 2Nal, wherein the 2Nal is an L-amino acid. In some embodiments, X₁₁ is d-2Nal.

In some embodiments, X₁₂ is THP, K—Z_{lipid}, Dab-Z_{lipid}, or NMeK—Z_{lipid}. In some embodiments, X₁₂ is THP, K—Z_{lipid}, Dab-Z_{lipid}, or NMeK—Z_{lipid}, wherein the K—Z_{lipid}, Dab-Z_{lipid}, and NMeK—Z_{lipid} are L amino acids. In some embodiments, X₁₂ is THP, k-Z_{lipid}, dDab-Z_{lipid}, or NMek-Z_{lipid}.

In some embodiments, X₁₂ is THP, K—Z_{lipid}, or NMeK—Z_{lipid}. In some embodiments, X₁₂ is THP, K—Z_{lipid}, or Dab-Z_{lipid}. In some embodiments, X₁₂ is THP or K—Z_{lipid}. In some embodiments, X₁₂ is THP. In some embodiments, X₁₂ is K—Z_{lipid}. In some embodiments, X₁₂ is k-Z_{lipid}. In some embodiments, X₁₂ is NMeK—Z_{lipid}. In some embodiments, X₁₂ is NMek-Z_{lipid}. In some embodiments, X₁₂ is Dab-Z_{lipid}. In some embodiments, X₁₂ is dDab-Z_{lipid}.

In some embodiments, X₁₃ is Dab(NMecarn), Dab-Z_{lipid}, E, E(OAll), K(Ac), K(d), K(NMeAc), K—Z_{peg}, K—Z_{lipid}, NMeK—Z_{lipid}, or absent. In some embodiments, X₁₃ is Dab(NMecarn), Dab-Z_{lipid}, E, E(OAll), K(Ac), K(d), K(NMeAc), K—Z_{peg}, K—Z_{lipid}, NMeK—Z_{lipid}, or absent, wherein the Dab(NMecarn), Dab-Z_{lipid}, E, E(OAll), K(Ac), K(d), K(NMeAc), K—Z_{peg}, K—Z_{lipid}, and NMeK—Z_{lipid} are L amino acids. In some embodiments, X₁₃ is dDab(NMecarn), dDab-Z_{lipid}, e, e(OAll), k(Ac), k(d), k(NMeAc), k-Z_{peg}, k-Z_{lipid}, NMek-Z_{lipid}, or absent.

In some embodiments, X₁₃ is Dab(NMecarn), Dab-Z_{lipid}, E, E(OAll), K(Ac), K(d), K(NMeAc), K—Z_{peg}, K—Z_{lipid}, or NMeK—Z_{lipid}. In some embodiments, X₁₃ is Dab-Z_{lipid}, E, K(Ac), K(d), K(NMeAc), K—Z_{peg}, K—Z_{lipid}, or NMeK—Z_{lipid}. In some embodiments, X₁₃ is E, K(Ac), K(d), K(NMeAc), K—Z_{peg}, K—Z_{lipid}, or NMeK—Z_{lipid}. In some embodiments, X₁₃ is E, K(d), K(NMeAc), K—Z_{peg}, or K—Z_{lipid}. In some embodiments, X₁₃ is E, K(NMeAc), or K—Z_{lipid}. In some embodiments, X₁₃ is E or K(NMeAc).

In some embodiments, X₁₃ is Dab(NMecarn), Dab-Z_{lipid}, E, E(OAll), K(Ac), K(d), K(NMeAc), K—Z_{peg}, K—Z_{lipid}, NMeK—Z_{lipid} or absent, wherein when X₁₃ is K—Z_{lipid}, Dab-Z_{lipid}, or NMeK—Z_{lipid}, then the lipophilic substituent is selected from PEG12gEC18OH, PEG12gEC20OH, PEG2PEG2gEC18OH, and PEG2PEG2gEC20OH. In some embodiments, X₁₃ is Dab(NMecarn), E, E(OAll), K(Ac), K(d), K(NMeAc), K—Z_{peg}, K—Z_{lipid}, NMeK—Z_{lipid} or absent, wherein when X₁₃ is K—Z_{lipid} or NMeK—Z_{lipid}, then the lipophilic substituent is selected from PEG12gEC18OH, PEG12gEC20OH, PEG2PEG2gEC18OH. In some embodiments, X₁₃ is Dab(NMecarn), Dab-Z_{lipid}, E, E(OAll), K(Ac), K(d), K(NMeAc), K—Z_{peg}, K—Z_{lipid}, or absent, wherein when X₁₃ is K—Z_{lipid} or Dab-Z_{lipid}, then the lipophilic substituent is selected from PEG12gEC18OH, PEG12gEC20OH, and PEG2PEG2gEC18OH. In some embodiments, X₁₃ is Dab(NMecarn), E, E(OAll), K(Ac), K(d), K(NMeAc), K—Z_{peg}, K—Z_{lipid} or absent, wherein when X₆ is K—Z_{lipid}, then the lipophilic substituent is selected from PEG12gEC18OH, PEG12gEC20OH, and PEG2PEG2gEC18OH.

In some embodiments, X₁₃ is Dab(NMecarn), Dab-Z_{lipid}, E, E(OAll), K(Ac), K(d), K(NMeAc), K—Z_{peg}, K—Z_{lipid} or NMeK—Z_{lipid}, wherein when X₁₃ is K—Z_{lipid}, Dab-Z_{lipid}, or NMeK—Z_{lipid}, then the lipophilic substituent is selected from PEG12gEC18OH, PEG12gEC20OH, PEG2PEG2gEC18OH, and PEG2PEG2gEC20OH. In some embodiments, X₁₃ is Dab(NMecarn), Dab-Z_{lipid}, E, E(OAll), K(Ac), K(d), K(NMeAc), K—Z_{peg}, K—Z_{lipid} or NMeK—Z_{lipid}, wherein when X₁₃ is K—Z_{lipid}, Dab-Z_{lipid}, or NMeK—Z_{lipid}, then the lipophilic substituent is selected from PEG12gEC18OH, PEG12gEC20OH, and PEG2PEG2gEC18OH. In some embodiments, X₁₃ is Dab(NMecarn), Dab-Z_{lipid}, E, E(OAll), K(Ac), K(d), K(NMeAc), K—Z_{peg}, K—Z_{lipid} or NMeK—Z_{lipid}, wherein when X₁₃ is K—Z_{lipid}, Dab-Z_{lipid}, or NMeK—Z_{lipid}, then the lipophilic substituent is selected from PEG12gEC18OH and PEG2PEG2gEC18OH. In some embodiments, X₁₃ is Dab(NMecarn), Dab-Z_{lipid}, E, E(OAll), K(Ac), K(d), K(NMeAc), K—Z_{peg}, K—Z_{lipid} or NMeK—Z_{lipid}, wherein when X₁₃ is K—Z_{lipid}, Dab-Z_{lipid}, or NMeK—Z_{lipid}, then the lipophilic substituent is selected from PEG12gEC20OH and PEG2PEG2gEC18OH. In some embodiments, X₁₃ is Dab(NMecarn), Dab-Z_{lipid}, E, E(OAll), K(Ac), K(d), K(NMeAc), K—Z_{peg}, K—Z_{lipid} or NMeK—Z_{lipid}, wherein when X₁₃ is K—Z_{lipid}, Dab-Z_{lipid}, or NMeK—Z_{lipid}, then the lipophilic substituent is PEG2PEG2gEC18OH.

In some embodiments, X₁₃ is E, K(NMeAc), or K—Z_{lipid}, wherein when X₁₃ is K—Z_{lipid}, then the lipophilic substituent is selected from PEG12gEC18OH, PEG12gEC20OH, PEG2PEG2gEC18OH, and PEG2PEG2gEC20OH. In some embodiments, X₁₃ is E, K(NMeAc), or K—Z_{lipid}, wherein when X₁₃ is K—Z_{lipid}, then the lipophilic substituent is selected from PEG12gEC18OH, PEG12gEC20OH, and PEG2PEG2gEC18OH. In some embodiments, X₁₃ is E, K(NMeAc), or K—Z_{lipid}, wherein when X₁₃ is K—Z_{lipid}, then the lipophilic substituent is selected from PEG12gEC18OH and PEG2PEG2gEC18OH. In some embodiments, X₁₃ is E, K(NMeAc), or K—Z_{lipid}, wherein when X₁₃ is K—Z_{lipid}, then the lipophilic substituent is PEG2PEG2gEC18OH.

In some embodiments, X₁₃ is Dab(NMecarn). In some embodiments, X₁₃ is Dab-Z_{lipid}. In some embodiments, X₁₃ is Dab-Z_{lipid}, wherein the lipophilic substituent is selected from PEG12gEC18OH, PEG12gEC20OH, PEG2PEG2gEC18OH, and PEG2PEG2gEC20OH. In some embodiments, X₁₃ is E. In some embodiments, X₁₃ is E(OAll). In some embodiments, X₁₃ is K(Ac). In some embodiments, X₁₃ is K(d). In some embodiments, X₁₃ is K(NMeAc). In some embodiments, X₁₃ is K—Z_{peg}. In some embodiments, X₁₃ is K—Z_{lipid}. In some embodiments, X₁₃ is K—Z_{lipid}, wherein the lipophilic substituent is selected from PEG12gEC18OH, PEG12gEC20OH, PEG2PEG2gEC18OH, and PEG2PEG2gEC20OH. In some embodiments, X₁₃ is NMeK—Z_{lipid}. In some embodiments, X₁₃ is NMeK—Z_{lipid}, wherein the lipophilic substituent is selected from PEG12gEC18OH, PEG12gEC20OH, PEG2PEG2gEC18OH, and PEG2PEG2gEC20OH.

In some embodiments, X₁₃ is dDab(NMecarn). In some embodiments, X₁₃ is dDab-Z_{lipid}. In some embodiments, X₁₃ is dDab-Z_{lipid}, wherein the lipophilic substituent is selected from PEG12gEC18OH, PEG12gEC20OH, PEG2PEG2gEC18OH, and PEG2PEG2gEC20OH. In some embodiments, X₁₃ is e. In some embodiments, X₁₃ is e(OAll). In some embodiments, X₁₃ is k(Ac). In some embodiments, X₁₃ is k(d). In some embodiments, X₁₃ is k(NMeAc). In some embodiments, X₁₃ is k-Z_{peg}. In some embodiments, X₁₃ is k-Z_{lipid}. In some embodiments, X₁₃ is k-Z_{lipid}, wherein the lipophilic substituent is selected from PEG12gEC18OH, PEG12gEC20OH, PEG2PEG2gEC18OH, and PEG2PEG2gEC20OH. In some embodiments, X₁₃ is NMek-Z_{lipid}. In some embodiments, X₁₃ is NMek-Z_{lipid}, wherein the lipophilic substituent is selected from PEG12gEC18OH, PEG12gEC20OH, PEG2PEG2gEC18OH, and PEG2PEG2gEC20OH.

In some embodiments, X₁₄ is N, K—Z_{lipid}, NMeK—Z_{lipid}, or Dab-Z_{lipid}. In some embodiments, X₁₄ is N, K—Z_{lipid}, NMeK—Z_{lipid}, or Dab-Z_{lipid}, each of which is an L-amino acid. In some embodiments, X₁₄ is n, k-Z_{lipid}, NMek-Z_{lipid}, or dDab-Z_{lipid}.

In some embodiments, X₁₄ is N, K—Z_{lipid} or NMeK—Z_{lipid}. In some embodiments, X₁₄ is N, K—Z_{lipid}, or Dab-Z_{lipid}. In some embodiments, X₁₄ is N or K—Z_{lipid}.

In some embodiments, X₁₄ is N. In some embodiments, X₁₄ is K—Z_{lipid}. In some embodiments, X₁₄ is NMeK—Z_{lipid}. In some embodiments, X₁₄ is Dab-Z_{lipid}. In some embodiments, X₁₄ is n. In some embodiments, X₁₄ is k-Z_{lipid}. In some embodiments, X₁₄ is NMek-Z_{lipid}. In some embodiments, X₁₄ is dDab-Z_{lipid}.

In some embodiments, X₁₅ is 3Pya. In some embodiments, X₁₅ is 3Pya, wherein the 3Pya is an L-amino acid. In some embodiments, X₁₅ is d-3Pya.

In some embodiments, X₁₆ is Sar, NMeK(d), K—Z_{lipid}, Dab-Z_{lipid}, or NMeK—Z_{lipid}. In some embodiments, X₁₆ is Sar, NMeK(d), K—Z_{lipid}, Dab-Z_{lipid}, or NMeK—Z_{lipid}, wherein the NMeK(d), K—Z_{lipid}, Dab-Z_{lipid}, and NMeK—Z_{lipid} are L-amino acids. In some embodiments, X₁₆ is Sar, NMek(d), k-Z_{lipid}, dDab-Z_{lipid}, or NMek-Z_{lipid}.

In some embodiments, X₁₆ is Sar, NMeK(d), K—Z_{lipid}, or NMeK—Z_{lipid}. In some embodiments, X₁₆ is Sar, NMeK(d), or NMeK—Z_{lipid}. In some embodiments, X₁₆ is Sar or NMeK—Z_{lipid}.

In some embodiments, X₁₆ is Sar, NMeK(d), K—Z_{lipid}, Dab-Z_{lipid}, or NMeK—Z_{lipid}, wherein when X₁₆ is K—Z_{lipid}, Dab-Z_{lipid}, or NMeK—Z_{lipid}, then the lipophilic substituent is selected from gEC18OH, PEG12C18OH, PEG6C18OH, PEG12gEC18OH, PEG6gEC18OH, PEG2PEG2gEC18OH, PEG2PEG2gEC20OH, PEG2NMePEG2NMegENMeC18OH, and PEG2PEG2gEC6X. In some embodiments, X₁₆ is Sar, NMeK(d), or NMeK—Z_{lipid}, wherein when X₁₆ is NMeK—Z_{lipid}, then the lipophilic substituent is selected from gEC18OH, PEG12C18OH, PEG6C18OH, PEG12gEC18OH, PEG6gEC18OH, PEG2PEG2gEC18OH, PEG2PEG2gEC20OH, PEG2NMePEG2NMegENMeC18OH, and PEG2PEG2gEC6X.

In some embodiments, X₁₆ is Sar, NMeK(d), K—Z_{lipid}, Dab-Z_{lipid}, or NMeK—Z_{lipid}, wherein when X₁₆ is K—Z_{lipid}, Dab-Z_{lipid}, or NMeK—Z_{lipid}, then the lipophilic substituent is selected from gEC18OH, PEG12gEC18OH, PEG6gEC18OH, PEG2PEG2gEC18OH, PEG2PEG2gEC20OH, and PEG2PEG2gEC6X. In some embodiments, X₁₆ is Sar, NMeK(d), or NMeK—Z_{lipid}, wherein when X₁₆ is NMeK—Z_{lipid}, then the lipophilic substituent is selected from gEC18OH, PEG12gEC18OH, PEG6gEC18OH, PEG2PEG2gEC18OH, PEG2PEG2gEC20OH, and PEG2PEG2gEC6X.

In some embodiments, X₁₆ is Sar, NMeK(d), K—Z_{lipid}, Dab-Z_{lipid}, or NMeK—Z_{lipid}, wherein when X₁₆ is K—Z_{lipid}, Dab-Z_{lipid}, or NMeK—Z_{lipid}, then the lipophilic substituent is selected from PEG12gEC18OH, PEG6gEC18OH, PEG2PEG2gEC18OH, and PEG2PEG2gEC20OH. In some embodiments, X₁₆ is Sar, NMeK(d), or NMeK—Z_{lipid}, wherein when X₁₆ is NMeK—Z_{lipid}, then the lipophilic substituent is selected from PEG12gEC18OH, PEG6gEC18OH, PEG2PEG2gEC18OH, and PEG2PEG2gEC20OH. In some embodiments, X₁₆ is Sar, NMeK(d), K—Z_{lipid}, Dab-Z_{lipid}, or NMeK—Z_{lipid}, wherein when X₁₆ is K—Z_{lipid}, Dab-Z_{lipid}, or NMeK—Z_{lipid}, then the lipophilic substituent is PEG2PEG2gEC18OH. In some embodiments, X₁₆ is Sar, NMeK(d), or NMeK—Z_{lipid}, wherein when X₁₆ is NMeK—Z_{lipid}, then the lipophilic substituent is PEG2PEG2gEC18OH.

In some embodiments, X₁₆ is Sar. In some embodiments, X₁₆ is Dab-Z_{lipid}. In some embodiments, X₁₆ is K—Z_{lipid}. In some embodiments, X₁₆ is NMeK(d). In some embodiments, X₁₆ is NMeK—Z_{lipid}. In some embodiments, X₁₆ is dDab-Z_{lipid}. In some embodiments, X₁₆ is k-Z_{lipid}. In some embodiments, X₁₆ is NMek(d). In some embodiments, X₁₆ is NMek-Z_{lipid}.

In some embodiments, X₁₇ is Dab-Z_{lipid}, K—Z_{lipid}, NMeK—Z_{lipid}, or absent. In some embodiments, X₁₇ is Dab-Z_{lipid}, K—Z_{lipid}, NMeK—Z_{lipid}, or absent, wherein the Dab-Z_{lipid}, K—Z_{lipid}, and NMeK—Z_{lipid} are L-amino acids. In some embodiments, X₁₇ is dDab-Z_{lipid}, k-Z_{lipid}, NMek-Z_{lipid}, or absent.

In some embodiments, X₁₇ is Dab-Z_{lipid}, K—Z_{lipid}, or NMeK—Z_{lipid}. In some embodiments, X₁₇ is K—Z_{lipid}, NMeK—Z_{lipid} or absent. In some embodiments, X₁₇ is K—Z_{lipid}, or absent.

In some embodiments, X₁₇ is Dab-Z_{lipid}, K—Z_{lipid}, NMeK—Z_{lipid} or absent, wherein when X₁₇ is Dab-Z_{lipid}, K—Z_{lipid}, or NMeK—Z_{lipid}, then the lipophilic substituent is selected from PEG12gEC18OH, PEG12gEC20OH, PEG2PEG2gEC18OH, PEG2PEG2gEC20OH, Sar-S-Sar-S-Sar-S-Sar-C₁₄, and PEG2NMePEG2NMegENMeC18Tetrazole. In some embodiments, X₁₇ is K—Z_{lipid}, or absent, wherein when X₁₇ is K—Z_{lipid}, or then the lipophilic substituent is selected from PEG12gEC18OH, PEG12gEC20OH, PEG2PEG2gEC18OH, PEG2PEG2gEC20OH, Sar-S-Sar-S-Sar-S-Sar-C₁₄, and PEG2NMePEG2NMegENMeC18Tetrazole.

In some embodiments, X₁₇ is Dab-Z_{lipid}, K—Z_{lipid}, NMeK—Z_{lipid} or absent, wherein when X₁₇ is Dab-Z_{lipid}, K—Z_{lipid}, or NMeK—Z_{lipid}, then the lipophilic substituent is selected from PEG12gEC18OH, PEG12gEC20OH, PEG2PEG2gEC18OH, and PEG2PEG2gEC20OH. In some embodiments, X₁₇ is K—Z_{lipid}, or absent, wherein when X₁₇ is K—Z_{lipid}, or then the lipophilic substituent is selected from PEG12gEC18OH, PEG12gEC20OH, PEG2PEG2gEC18OH, and PEG2PEG2gEC20OH.

In some embodiments, X₁₇ is Dab-Z_{lipid}, K—Z_{lipid}, NMeK—Z_{lipid} or absent, wherein when X₁₇ is Dab-Z_{lipid}, K—Z_{lipid}, or NMeK—Z_{lipid}, then the lipophilic substituent is selected from PEG12gEC18OH, PEG12gEC20OH, and PEG2PEG2gEC18OH. In some embodiments, X₁₇ is K—Z_{lipid}, or absent, wherein when X₁₇ is K—Z_{lipid}, or then the lipophilic substituent is selected from PEG12gEC18OH, PEG12gEC20OH and PEG2PEG2gEC18OH.

In some embodiments, X₁₇ is Dab-Z_{lipid}, K—Z_{lipid}, NMeK—Z_{lipid} or absent, wherein when X₁₇ is Dab-Z_{lipid}, K—Z_{lipid}, or NMeK—Z_{lipid}, then the lipophilic substituent is selected from PEG12gEC18OH and PEG2PEG2gEC18OH. In some embodiments, X₁₇ is Dab-Z_{lipid}, K—Z_{lipid}, NMeK—Z_{lipid} or absent, wherein when X₁₇ is Dab-Z_{lipid}, K—Z_{lipid}, or NMeK—Z_{lipid}, then the lipophilic substituent is selected from PEG12gEC20OH and PEG2PEG2gEC18OH. In some embodiments, X₁₇ is K—Z_{lipid}, or absent, wherein when X₁₇ is K—Z_{lipid}, or then the lipophilic substituent is selected from PEG12gEC20OH and PEG2PEG2gEC18OH.

In some embodiments, X₁₇ is Dab-Z_{lipid}. In some embodiments, X₁₇ is K—Z_{lipid}. In some embodiments, X₁₇ is K—Z_{lipid}, wherein the lipophilic substituent is selected from PEG12gEC18OH, PEG12gEC20OH, PEG2PEG2gEC18OH, PEG2PEG2gEC20OH, and PEG2NMePEG2NMegENMeC18Tetrazole. In some embodiments, X₁₇ is K—Z_{lipid}, wherein the lipophilic substituent is PEG2PEG2gEC18OH. In some embodiments, X₁₇ is NMeK—Z_{lipid}. In some embodiments, X₁₇ is absent.

In some embodiments, X₁₇ is dDab-Z_{lipid}. In some embodiments, X₁₇ is k-Z_{lipid}. In some embodiments, X₁₇ is k-Z_{lipid}, wherein the lipophilic substituent is selected from PEG12gEC18OH, PEG12gEC20OH, PEG2PEG2gEC18OH, PEG2PEG2gEC20OH, and PEG2NMePEG2NMegENMeC18Tetrazole. In some embodiments, X₁₇ is k-Z_{lipid}, wherein the lipophilic substituent is PEG2PEG2gEC18OH. In some embodiments, X₁₇ is NMek-Z_{lipid}.

In some embodiments, R₂ is CONH2, CON(Me)₂, a polyethylene glycol chain terminating in an ammonium group, or a lipophilic substituent.

In some embodiments, R₂ is CONH2, CON(Me)₂, or Z_{peg}. In some embodiments, R₂ is CONH2, CON(Me)₂, or Z_{lipid}. In some embodiments, R₂ is CONH2, CON(Me)₂, or Z_{lipid}, wherein the lipophilic substituent is selected from PEG12gEC18OH and PEG2PEG2gEC18OH.

In some embodiments, R₂ is CONH2 or CON(Me)₂.

In some embodiments, R₂ is CONH2. In some embodiments, R₂ is CON(Me)₂. In some embodiments, R₂ is Z_{peg}. In some embodiments, R₂ is Z_{lipid}.

In some embodiments, X₄ is Abu and X₉ is C. In some embodiments, X₄ is Abu and X₉ is aMeC. In some embodiments, X₄ is Abu and X₉ is Pen.

In some embodiments, X₄ is C and X₉ is C. In some embodiments, X₄ is C and X₉ is aMeC. In some embodiments, X₄ is C and X₉ is Pen.

In some embodiments, X₄ is Pen and X₉ is C. In some embodiments, X₄ is Pen and X₉ is aMeC. In some embodiments, X₄ is Pen and X₉ is Pen.

In some embodiments, X₄ is aMeC and X₉ is C. In some embodiments, X₄ is aMeC and X₉ is aMeC. In some embodiments, X₄ is aMeC and X₉ is Pen.

In some embodiments, X₄ is Pen(oXyl) and X₉ is C. In some embodiments, X₄ is Pen(oXyl) and X₉ is aMeC. In some embodiments, X₄ is Pen(oXyl) and X₉ is Pen.

In some embodiments, X₄ is Pen(mXyl) and X₉ is C. In some embodiments, X₄ is Pen(mXyl) and X₉ is aMeC. In some embodiments, X₄ is Pen(mXyl) and X₉ is Pen.

In some embodiments, X₄ is Pen(pXyl) and X₉ is C. In some embodiments, X₄ is Pen(pXyl) and X₉ is aMeC. In some embodiments, X₄ is Pen(pXyl) and X₉ is Pen.

In some embodiments, X₄ is 4AminoPro and X₉ is D. In some embodiments, X₄ is 4AminoPro and X₉ is E. In some embodiments, X₄ is 4AminoPro and X₉ is hE.

In some embodiments, X₄ is Dap and X₉ is D. In some embodiments, X₄ is Dap and X₉ is E. In some embodiments, X₄ is Dap and X₉ is hE.

In some embodiments, X₄ is Pra and X₉ is Dap(N3). In some embodiments, X₄ is aG and X₉ is aG.

In some embodiments, the peptide is cyclized via a linkage between two amino acid residues (e.g., at X₄ and X₉) having a structure selected from the following:

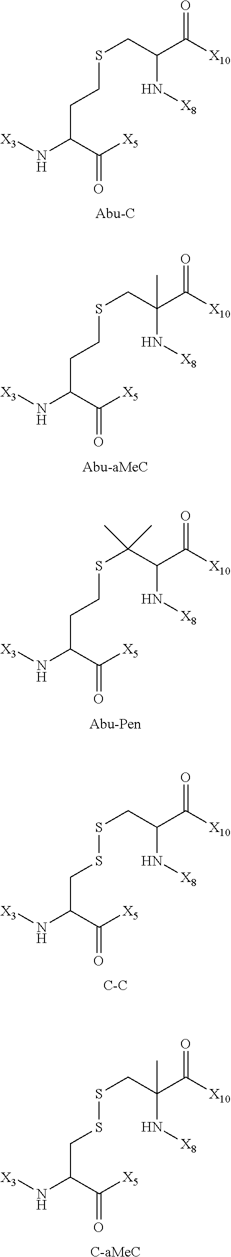

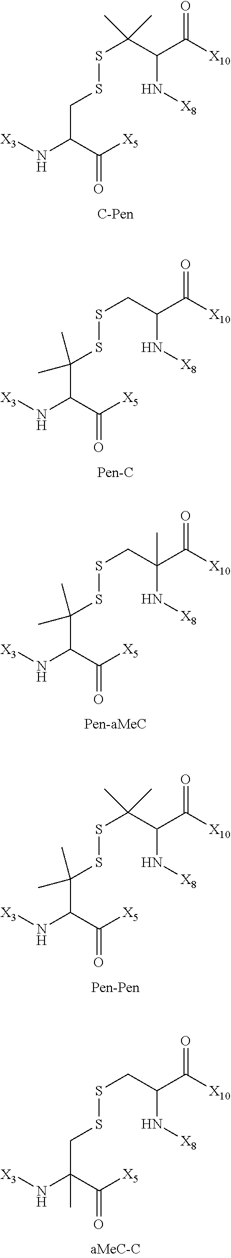

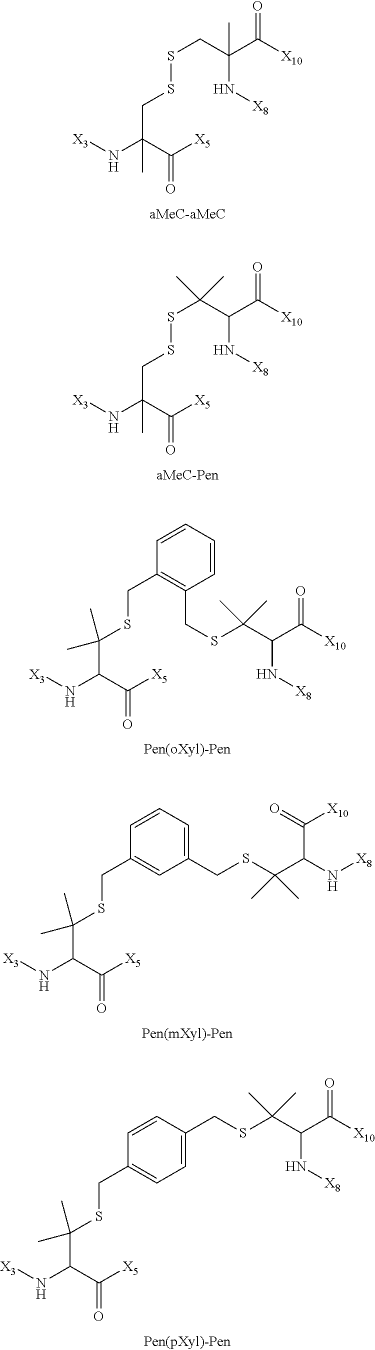

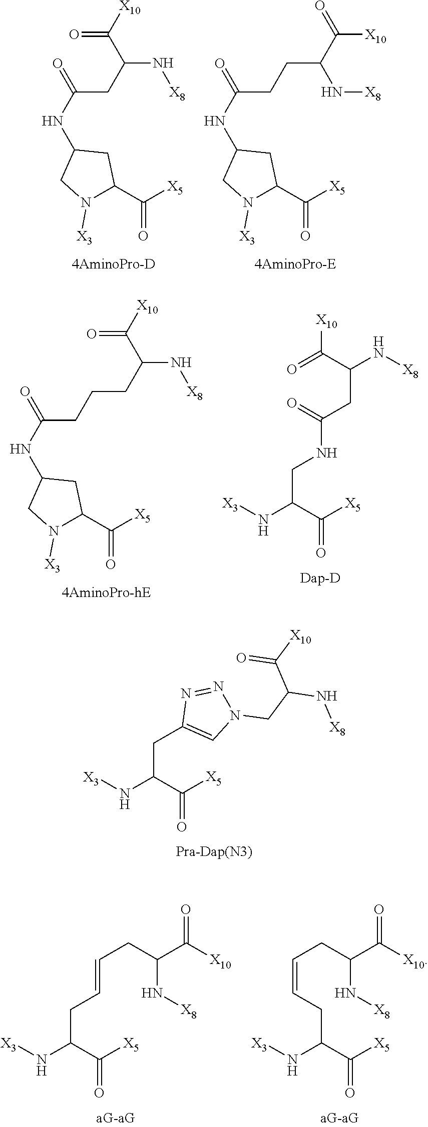

In some embodiments, the peptide is cyclized via a linkage between the residues at X₄ and X₉ having a structure selected from the following:

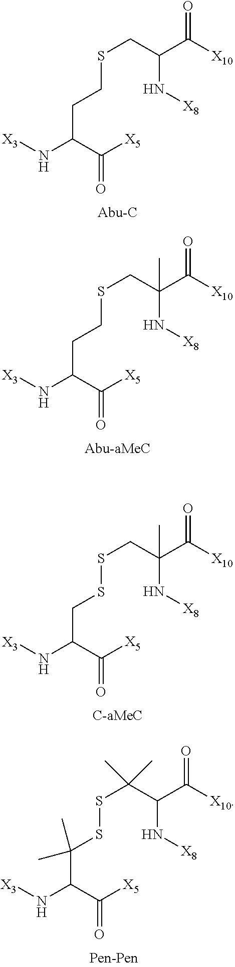

In some embodiments, the peptide is cyclized via a linkage between the residues at X₄ and X₉ having the following structure:

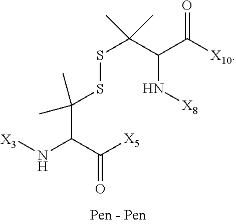

Non-limiting examples of ring-forming amino acids include 4AminoPro, Abu, aG, aMeC, Api, C, D, Dap, Dap(N3), Dab, E, hA, hE, hK, K, Orn, Pen, Pen(oXyl), Pen(mXyl), Pen(pXyl), Pra, R5H, R7H, R5Me, S5H, S7H, or S5Me.

In some embodiments, the ring comprises 4-9 or 11 amino acids. In some embodiments, the ring comprises 4, 6, or 10 amino acids. In some embodiments, the ring comprises 4 amino acids. In some embodiments, the ring comprises 5 amino acids. In some embodiments, the ring comprises 6 amino acids. In some embodiments, the ring comprises 7 amino acids. In some embodiments, the ring comprises 8 amino acids. In some embodiments, the ring comprises 9 amino acids. In some embodiments, the ring comprises 10 amino acids. In some embodiments, the ring comprises 11 amino acids. In some embodiments, the ring comprises 14 amino acids.

In some embodiments, the ring comprises a linkage between two ring-forming amino acids having a structure selected from the following:

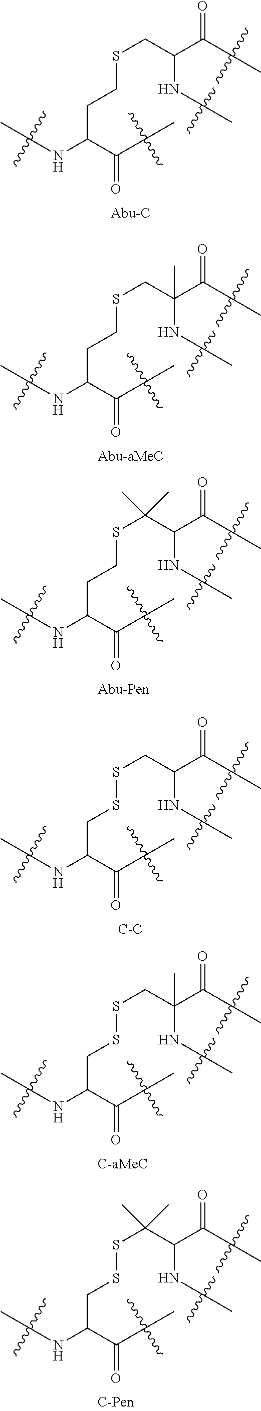

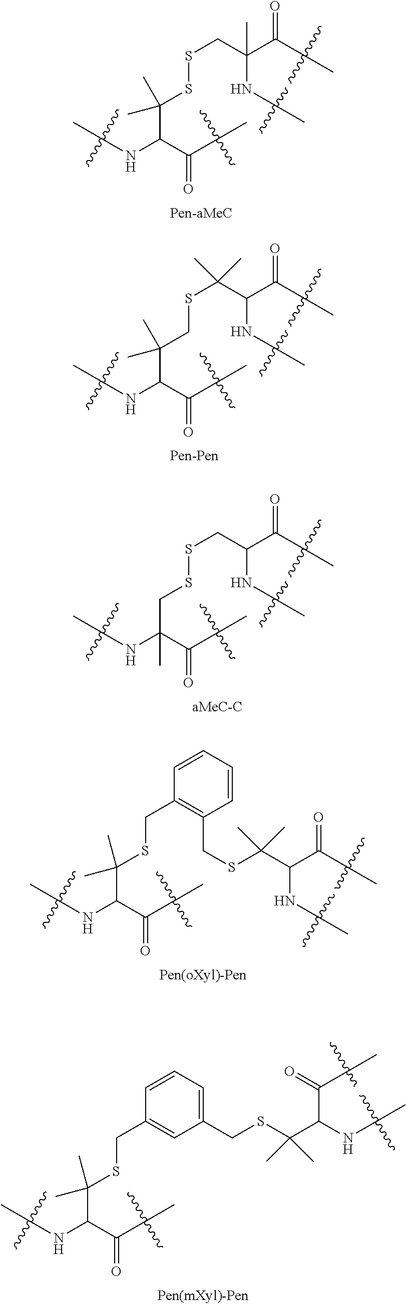

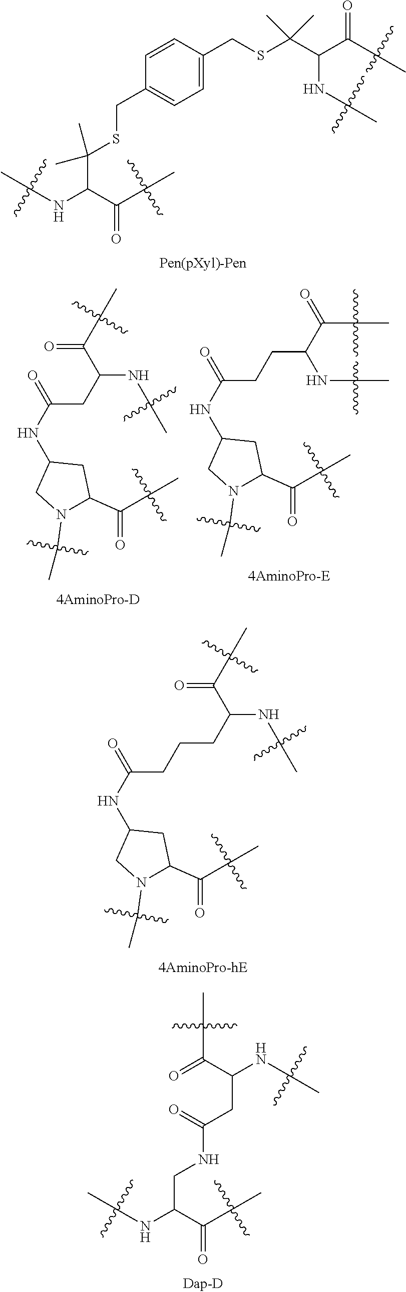 [SMILES: *NC(C(*)=O)C(C)(C)SCc1ccc(CSC(C)(C)C(N*)C(*)=O)cc1 ; *NC(CC(=O)NC1CC(C(*)=O)N(*)C1)C(*)=O ; *NC(CCC(=O)NC1CC(C(*)=O)N(*)C1)C(*)=O ; *NC(CCCC(=O)NC1CC(C(*)=O)N(*)C1)C(*)=O ; *NC(CNC(=O)CC(N*)C(*)=O)C(*)=O]

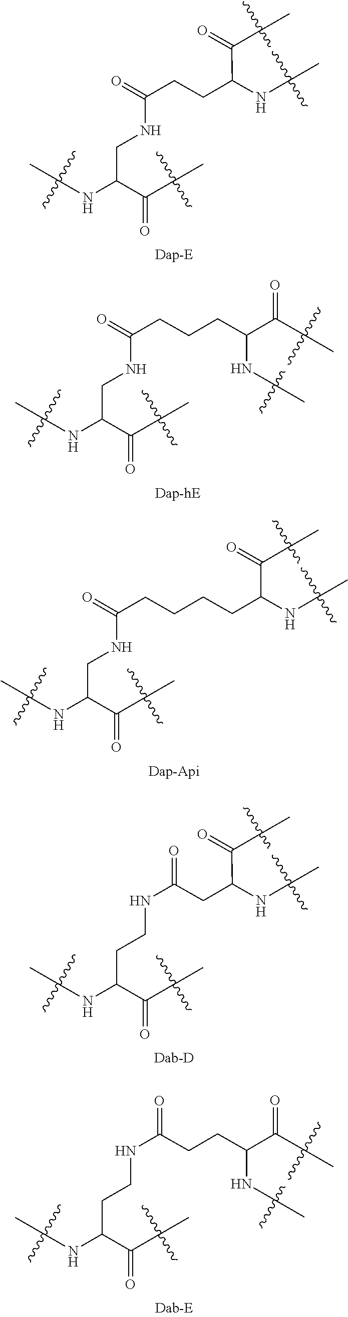 [SMILES: *NC(CCC(=O)NCC(N*)C(*)=O)C(*)=O ; *NC(CCCC(=O)NCC(N*)C(*)=O)C(*)=O ; *NC(CCCCC(=O)NCC(N*)C(*)=O)C(*)=O ; *NC(CCNC(=O)CC(N*)C(*)=O)C(*)=O ; *NC(CCNC(=O)CCC(N*)C(*)=O)C(*)=O]

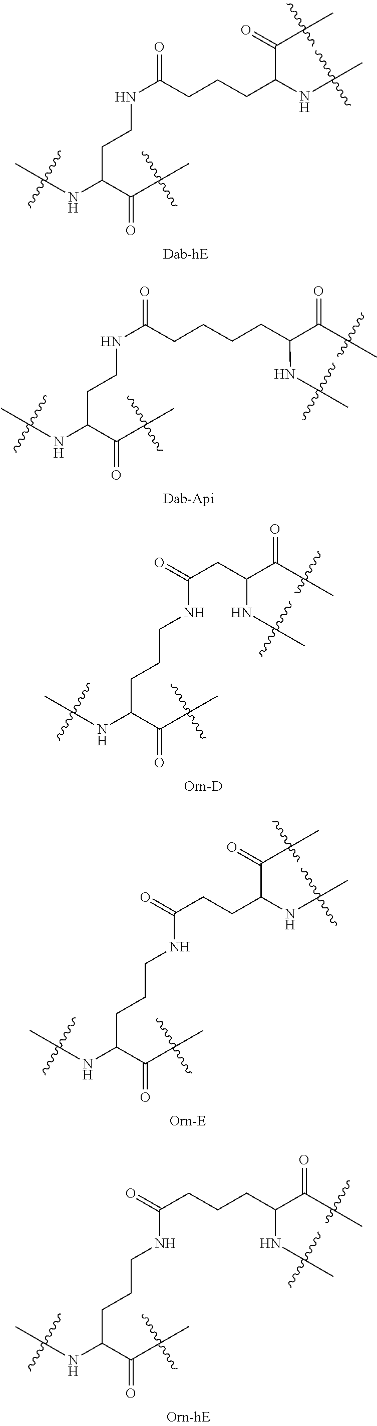 [SMILES: *NC(CCCC(=O)NCCC(N*)C(*)=O)C(*)=O ; *NC(CCCCC(=O)NCCC(N*)C(*)=O)C(*)=O ; *NC(CCCNC(=O)CC(N*)C(*)=O)C(*)=O ; *NC(CCCNC(=O)CCC(N*)C(*)=O)C(*)=O ; *NC(CCCNC(=O)CCCC(N*)C(*)=O)C(*)=O]

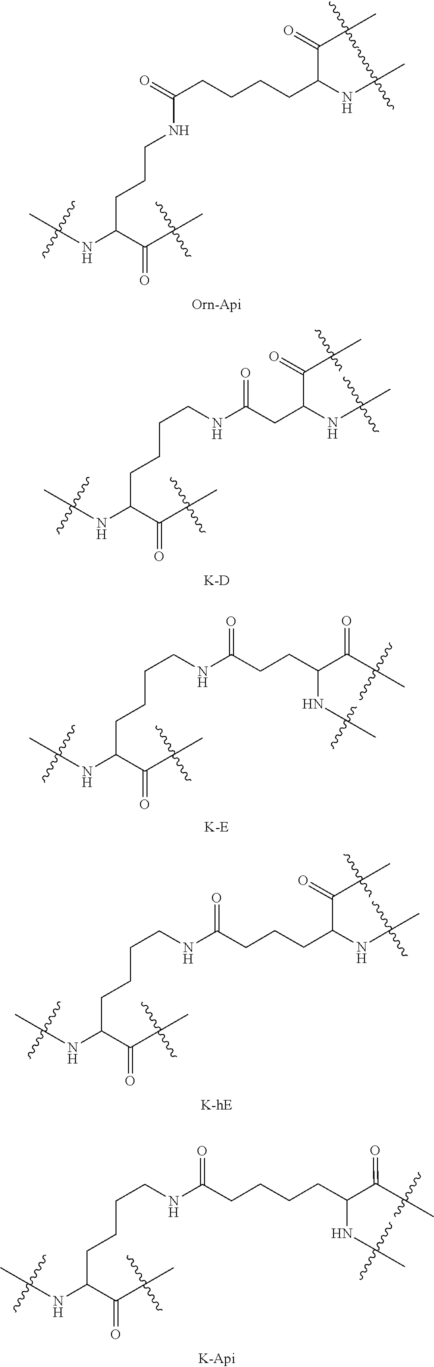

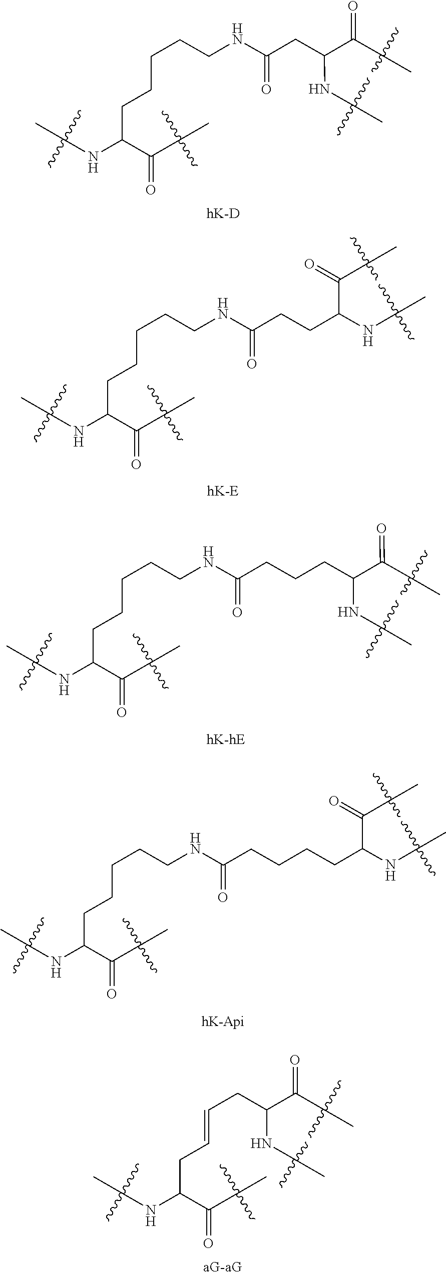 [SMILES: *NC(CCCCCNC(=O)CC(N*)C(*)=O)C(*)=O ; *NC(CCCCCNC(=O)CCC(N*)C(*)=O)C(*)=O ; *NC(CCCCCNC(=O)CCCC(N*)C(*)=O)C(*)=O ; *NC(CCCCCNC(=O)CCCCC(N*)C(*)=O)C(*)=O ; *NC(C/C=C/CC(N*)C(*)=O)C(*)=O]

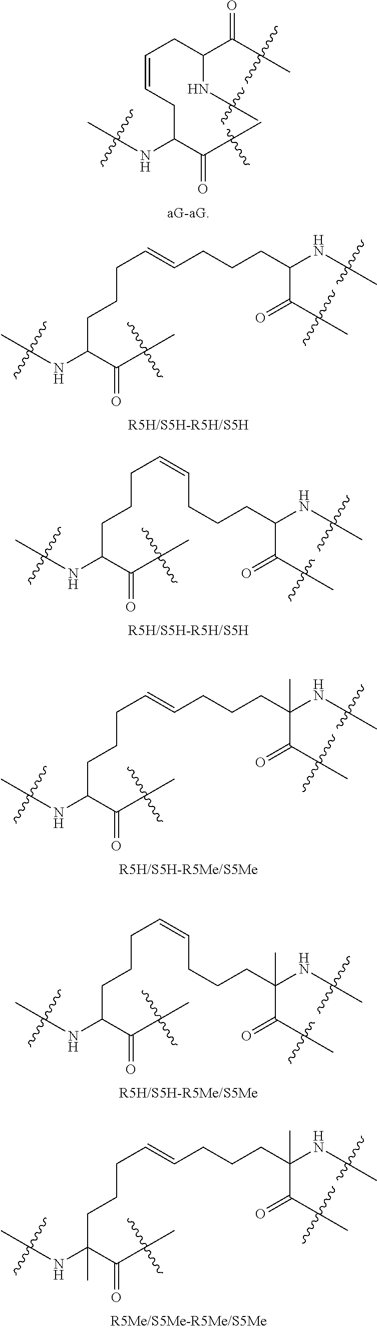 [SMILES: *NC(C/C=C\CC(N*)C(*)=O)C(*)=O ; *NC(CCC/C=C/CCCC(N*)C(*)=O)C(*)=O ; *NC(CCC/C=C\CCCC(N*)C(*)=O)C(*)=O ; *NC(CCC/C=C/CCCC(C)(N*)C(*)=O)C(*)=O ; *NC(CCC/C=C\CCCC(C)(N*)C(*)=O)C(*)=O ; *NC(C)(CCC/C=C/CCCC(C)(N*)C(*)=O)C(*)=O]

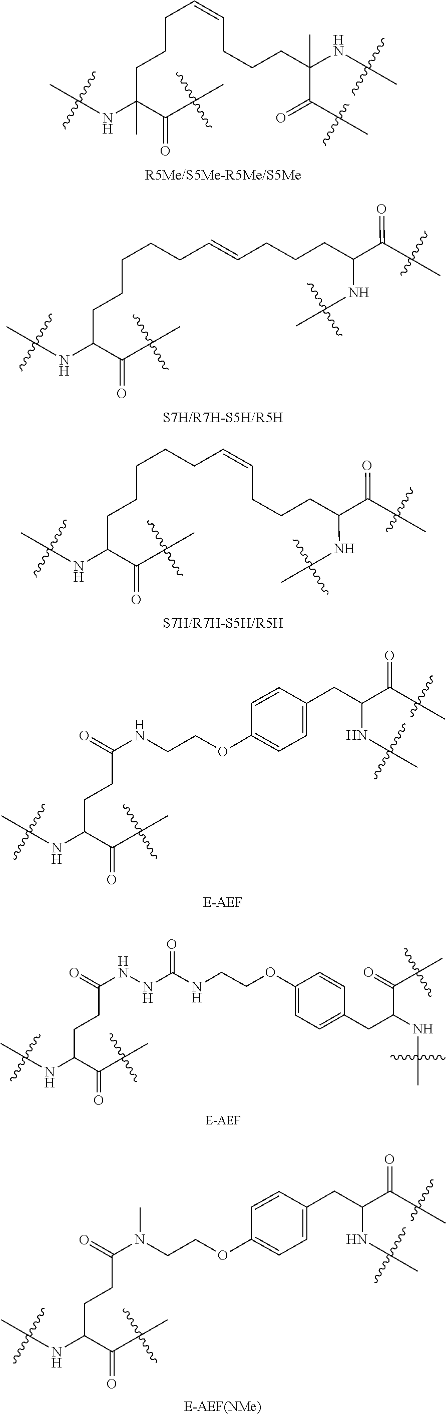 [SMILES: *NC(C)(CCC/C=C\CCCC(C)(N*)C(*)=O)C(*)=O ; *NC(CCC/C=C/CCCCCC(N*)C(*)=O)C(*)=O ; *NC(CCC/C=C\CCCCCC(N*)C(*)=O)C(*)=O ; *NC(CCC(=O)NCCOc1ccc(CC(N*)C(*)=O)cc1)C(*)=O ; *NC(CCC(=O)NNC(=O)NCCOc1ccc(CC(N*)C(*)=O)cc1)C(*)=O ; *NC(CCC(=O)N(C)CCOc1ccc(CC(N*)C(*)=O)cc1)C(*)=O]

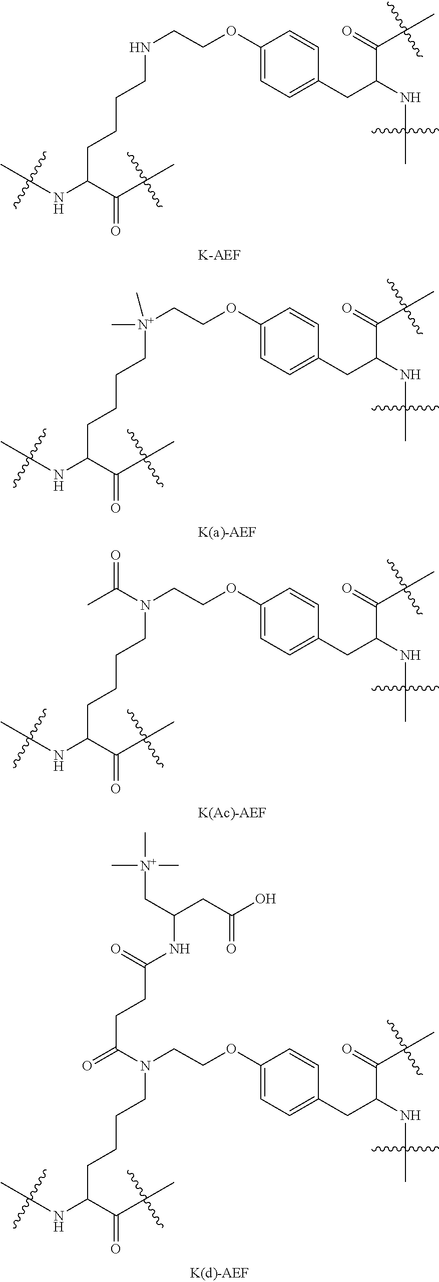 [SMILES: *NC(CCCCNCCOc1ccc(CC(N*)C(*)=O)cc1)C(*)=O ; *NC(CCCC[N+](C)(C)CCOc1ccc(CC(N*)C(*)=O)cc1)C(*)=O ; *NC(CCCCN(CCOc1ccc(CC(N*)C(*)=O)cc1)C(C)=O)C(*)=O ; *NC(CCCCN(CCOc1ccc(CC(N*)C(*)=O)cc1)C(=O)CCC(=O)NC(CC(=O)O)C[N+](C)(C)C)C(*)=O]

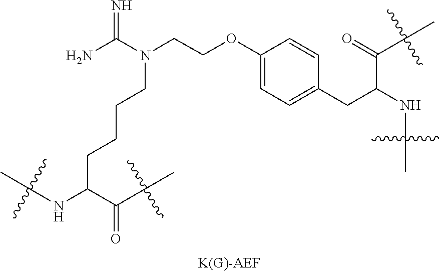 [SMILES: *NC(CCCCN(CCOc1ccc(CC(N*)C(*)=O)cc1)C(=N)N)C(*)=O]

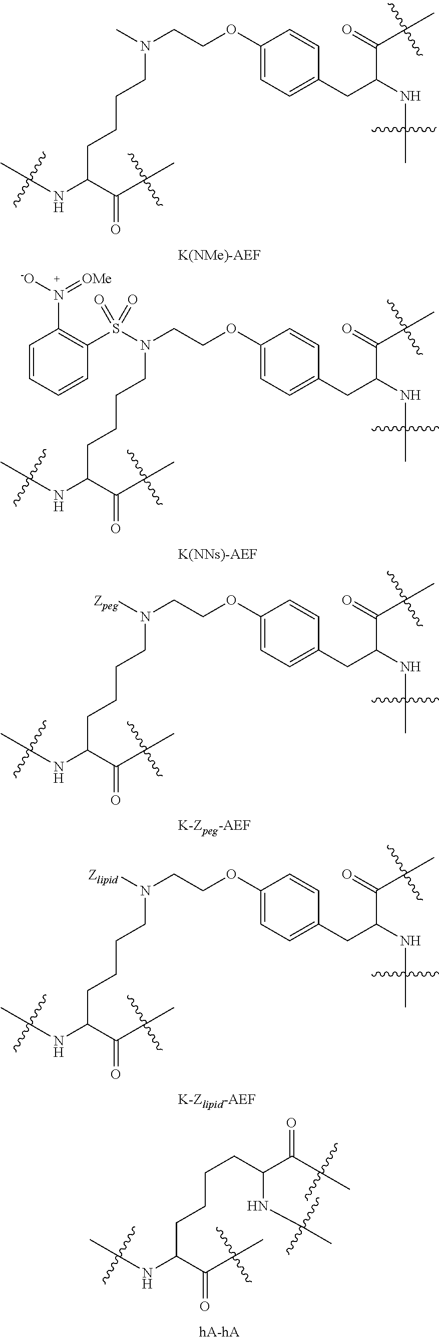

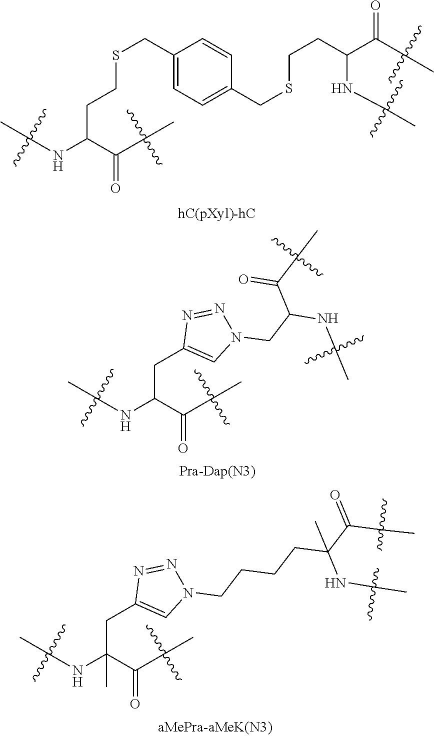 [SMILES: *NC(CCSCc1ccc(CSCCC(N*)C(*)=O)cc1)C(*)=O ; *NC(Cc1cn(CC(N*)C(*)=O)nn1)C(*)=O ; *NC(C)(CCCCn1cc(CC(C)(N*)C(*)=O)nn1)C(*)=O]

In some embodiments, the ring comprises a linkage between the N-terminus of the peptide and a ring-forming amino acid and has a structure selected from the following:

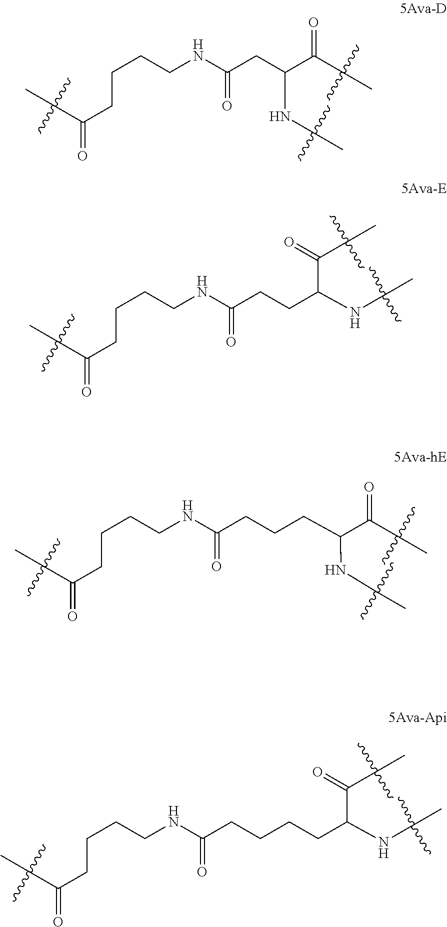 [SMILES: *NC(CC(=O)NCCCCC(*)=O)C(*)=O ; *NC(CCC(=O)NCCCCC(*)=O)C(*)=O ; *NC(CCCC(=O)NCCCCC(*)=O)C(*)=O ; *NC(CCCCC(=O)NCCCCC(*)=O)C(*)=O]

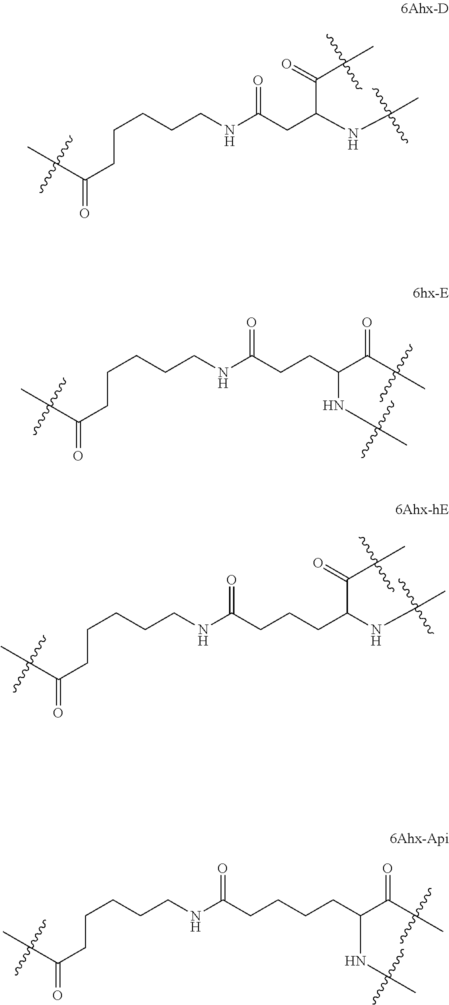 [SMILES: *NC(CC(=O)NCCCCCC(*)=O)C(*)=O ; *NC(CCC(=O)NCCCCCC(*)=O)C(*)=O ; *NC(CCCC(=O)NCCCCCC(*)=O)C(*)=O ; *NC(CCCCC(=O)NCCCCCC(*)=O)C(*)=O]

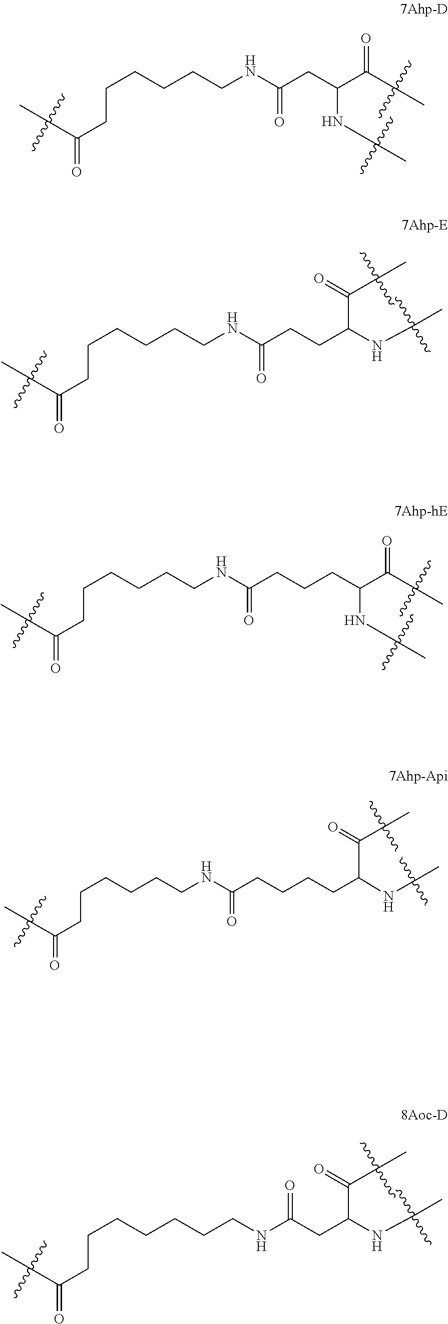 [SMILES: *NC(CC(=O)NCCCCCCC(*)=O)C(*)=O ; *NC(CCC(=O)NCCCCCCC(*)=O)C(*)=O ; *NC(CCCC(=O)NCCCCCCC(*)=O)C(*)=O ; *NC(CCCCC(=O)NCCCCCCC(*)=O)C(*)=O ; *NC(CC(=O)NCCCCCCCC(*)=O)C(*)=O]

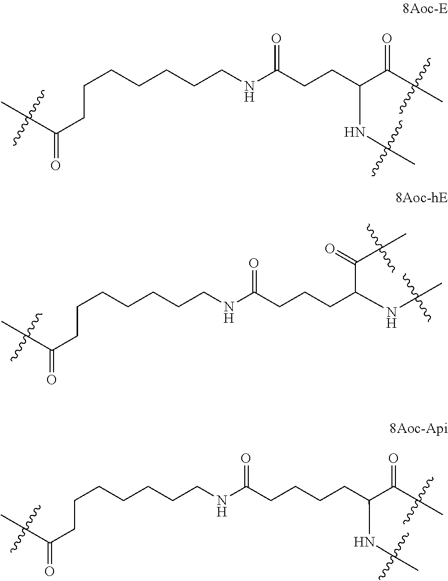 [SMILES: *NC(CCC(=O)NCCCCCCCC(*)=O)C(*)=O ; *NC(CCCC(=O)NCCCCCCCC(*)=O)C(*)=O ; *NC(CCCCC(=O)NCCCCCCCC(*)=O)C(*)=O]

In some embodiments, the ring is formed between X₄ and X₉, X₄ and X₁₃, X₅ and X₁₀, X₃ and X₁₃, or X₆ and X₉. In some embodiments, the ring is formed between X₄ and X₉, X₄ and X₁₃, X₅ and X₁₀, X₃ and X₁₃, or X₆ and X₉ via a linker having one or more groups selected from the group consisting of a disulfide, thioether, amide, olefin, ether, alkylene, and triazole. In some embodiments, the ring is formed between X₄ and X₉, X₄ and X₁₃, X₅ and X₁₀, X₃ and X₁₃, or X₆ and X₉ via a linker selected from the group consisting of a disulfide, thioether, amide, olefin, and triazole.

In some embodiments, the ring is formed between X₄ and X₉, X₄ and X₁₃, or X₆ and X₉. In some embodiments, the ring is formed between X₄ and X₉, X₄ and X₁₃, or X₆ and X₉ via a linker having one or more groups selected from the group consisting of a disulfide, thioether, amide, olefin, ether, alkylene, and triazole. In some embodiments, the ring is formed between X₄ and X₉, X₄ and X₁₃, or X₆ and X₉ via a linker selected from the group consisting of a disulfide, thioether, amide, olefin, and triazole.

In some embodiments, the ring is formed between X₄ and X₉. In some embodiments, the ring is formed between X₄ and X₉ via a linker having one or more groups selected from the group consisting of a disulfide, thioether, amide, olefin, ether, alkylene, and triazole. In some embodiments, the ring is formed between X₄ and X₉ via a linker selected from the group consisting of a disulfide, thioether, amide, olefin, and triazole.

In some embodiments, the ring is formed between X₄ and X₁₃. In some embodiments, the ring is formed between X₄ and X₁₃ via a linker having one or more groups selected from the group consisting of a disulfide, thioether, amide, olefin, ether, alkylene, and triazole. In some embodiments, the ring is formed between X₄ and X₁₃ via a linker selected from the group consisting of a disulfide, thioether, amide, olefin, and triazole.

In some embodiments, the ring is formed between X₅ and X₁₀. In some embodiments, the ring is formed between X₅ and X₁₀ via a linker having one or more groups selected from the group consisting of a disulfide, thioether, amide, olefin, ether, alkylene, and triazole. In some embodiments, the ring is formed between X₅ and X₁₀ via a linker selected from the group consisting of a disulfide, thioether, amide, olefin, and triazole.

In some embodiments, the ring is formed between X₃ and X₁₃. In some embodiments, the ring is formed between X₃ and X₁₃ via a linker having one or more groups selected from the group consisting of a disulfide, thioether, amide, olefin, ether, alkylene, and triazole. In some embodiments, the ring is formed between X₃ and X₁₃ via a linker selected from the group consisting of a disulfide, thioether, amide, olefin, and triazole.

In some embodiments, the ring is formed between X₆ and X₉. In some embodiments, the ring is formed between X₆ and X₉ via a linker having one or more groups selected from the group consisting of a disulfide, thioether, amide, olefin, ether, alkylene, and triazole. In some embodiments, the ring is formed between X₆ and X₉ via a linker selected from the group consisting of a disulfide, thioether, amide, olefin, and triazole.

In some embodiments, the polypeptide comprises a second ring comprising 4-11 or 14 amino acids wherein the second ring is formed via a linkage between a third ring-forming amino acid and a fourth ring-forming amino acid or via a linkage between a third amino acid and the C-terminus of the polypeptide. In some embodiments, the second ring comprises 4-9 or 11 amino acids. In some embodiments, the second ring comprises 4, 6, 10 or 11 amino acids. In some embodiments, the second ring comprises 4 amino acids. In some embodiments, the second ring comprises 5 amino acids. In some embodiments, the second ring comprises 6 amino acids. In some embodiments, the second ring comprises 7 amino acids. In some embodiments, the second ring comprises 8 amino acids. In some embodiments, the second ring comprises 9 amino acids. In some embodiments, the second ring comprises 10 amino acids. In some embodiments, the second ring comprises 11 amino acids. In some embodiments, the second ring comprises 14 amino acids.

In some embodiments, the second ring comprises a linkage between two ring-forming amino acids having a structure selected from the following:

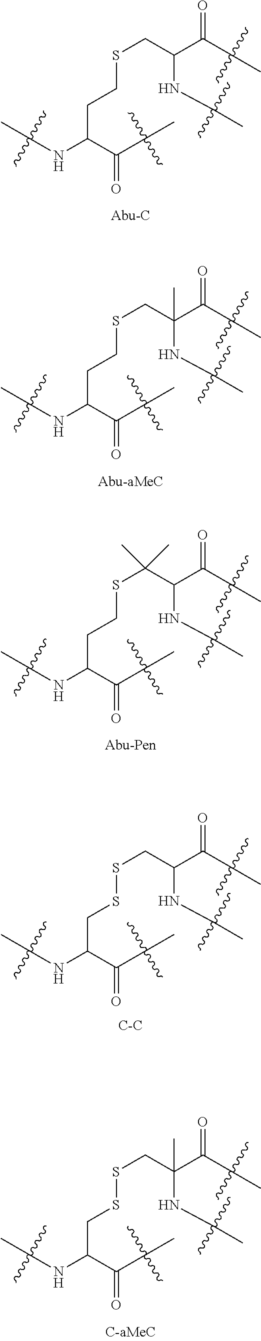

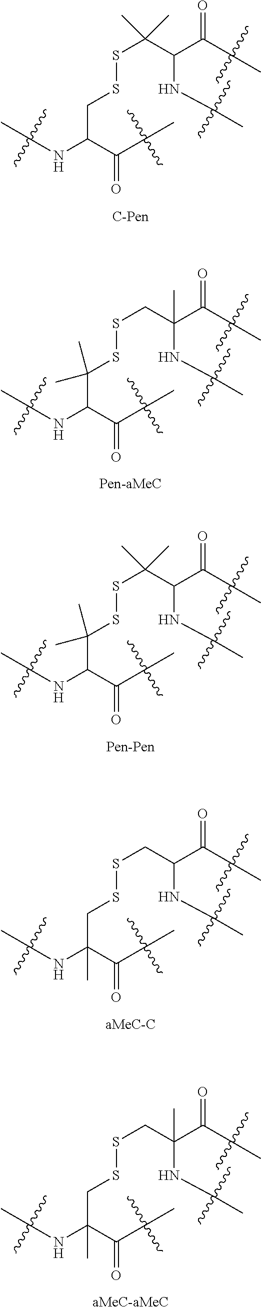 [SMILES: *NC(CSSC(C)(C)C(N*)C(*)=O)C(*)=O ; *NC(C(*)=O)C(C)(C)SSCC(C)(N*)C(*)=O ; *NC(C(*)=O)C(C)(C)SSC(C)(C)C(N*)C(*)=O ; *NC(CSSCC(C)(N*)C(*)=O)C(*)=O ; *NC(C)(CSSCC(C)(N*)C(*)=O)C(*)=O]

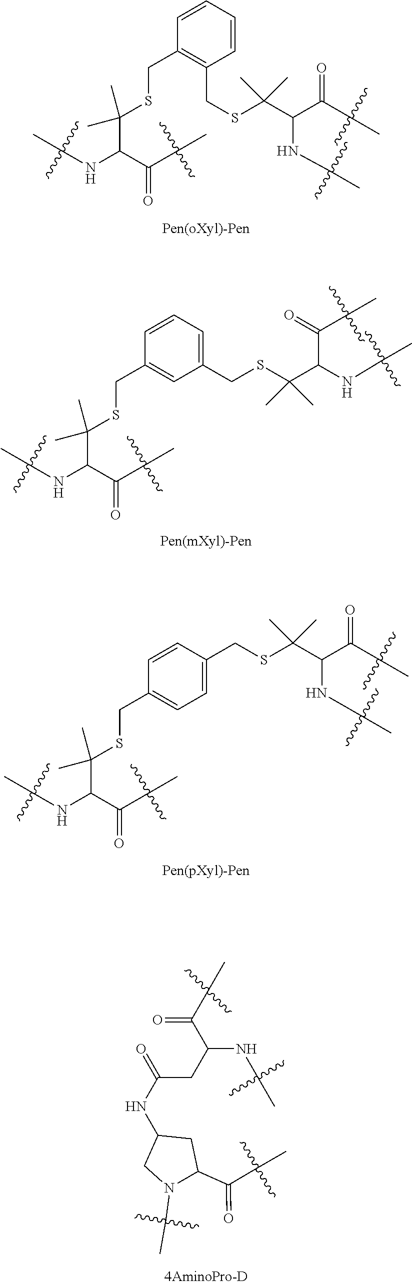 [SMILES: *NC(C(*)=O)C(C)(C)SCc1ccccc1CSC(C)(C)C(N*)C(*)=O ; *NC(C(*)=O)C(C)(C)SCc1cccc(CSC(C)(C)C(N*)C(*)=O)c1 ; *NC(C(*)=O)C(C)(C)SCc1ccc(CSC(C)(C)C(N*)C(*)=O)cc1 ; *NC(CC(=O)NC1CC(C(*)=O)N(*)C1)C(*)=O]

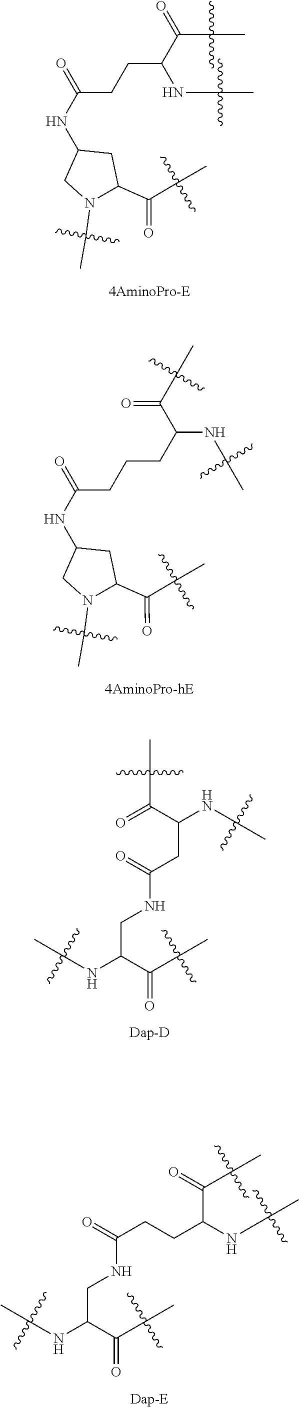 [SMILES: *NC(CCC(=O)NC1CC(C(*)=O)N(*)C1)C(*)=O ; *NC(CCCC(=O)NC1CC(C(*)=O)N(*)C1)C(*)=O ; *NC(CNC(=O)CC(N*)C(*)=O)C(*)=O ; *NC(CCC(=O)NCC(N*)C(*)=O)C(*)=O]

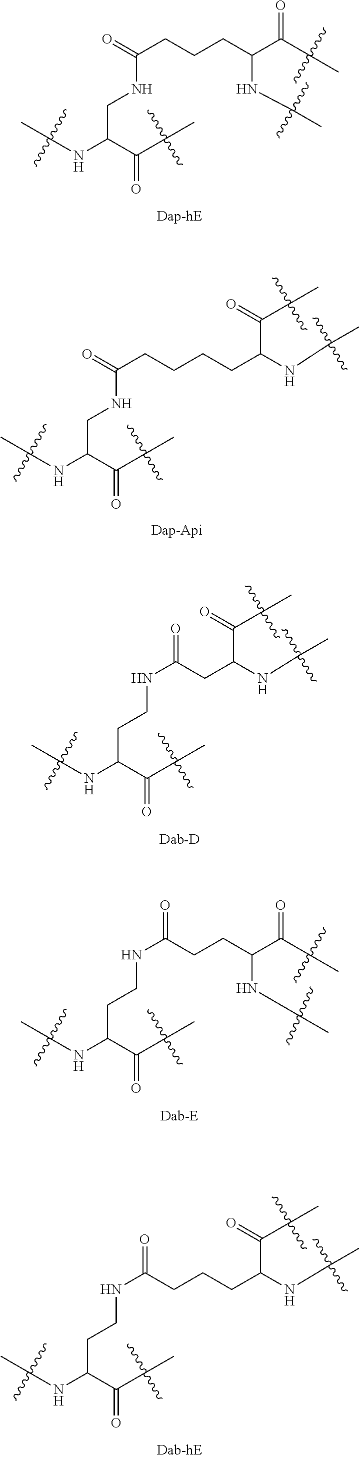 [SMILES: *NC(CCCC(=O)NCC(N*)C(*)=O)C(*)=O ; *NC(CCCCC(=O)NCC(N*)C(*)=O)C(*)=O ; *NC(CCNC(=O)CC(N*)C(*)=O)C(*)=O ; *NC(CCNC(=O)CCC(N*)C(*)=O)C(*)=O ; *NC(CCCC(=O)NCCC(N*)C(*)=O)C(*)=O]

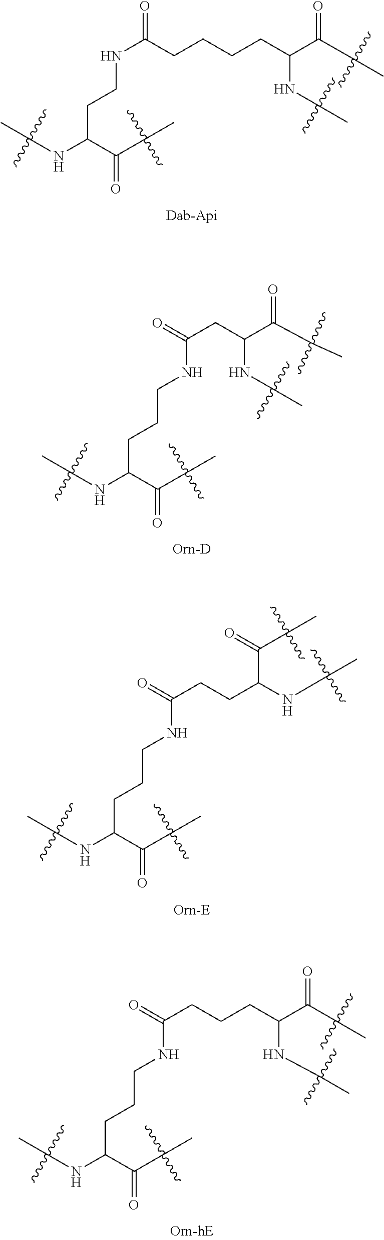 [SMILES: *NC(CCCCC(=O)NCCC(N*)C(*)=O)C(*)=O ; *NC(CCCNC(=O)CC(N*)C(*)=O)C(*)=O ; *NC(CCCNC(=O)CCC(N*)C(*)=O)C(*)=O ; *NC(CCCNC(=O)CCCC(N*)C(*)=O)C(*)=O]

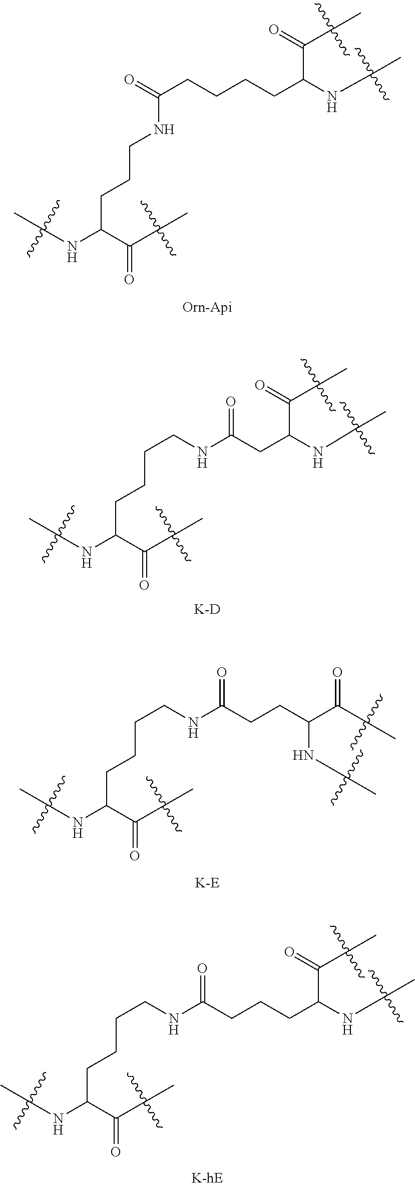

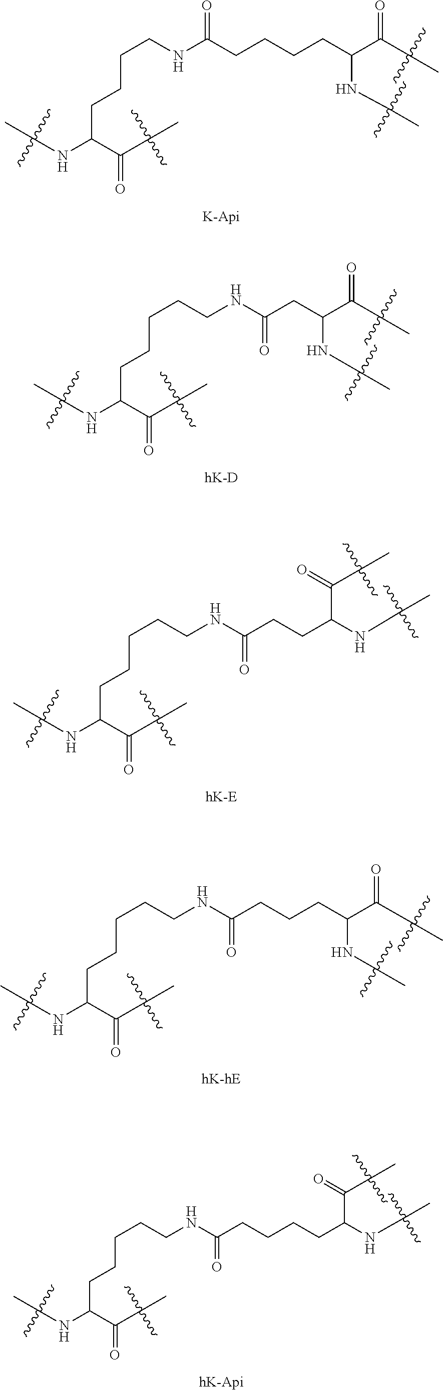 [SMILES: *NC(CCCCNC(=O)CCCCC(N*)C(*)=O)C(*)=O ; *NC(CCCCCNC(=O)CC(N*)C(*)=O)C(*)=O ; *NC(CCCCCNC(=O)CCC(N*)C(*)=O)C(*)=O ; *NC(CCCCCNC(=O)CCCC(N*)C(*)=O)C(*)=O ; *NC(CCCCCNC(=O)CCCCC(N*)C(*)=O)C(*)=O]

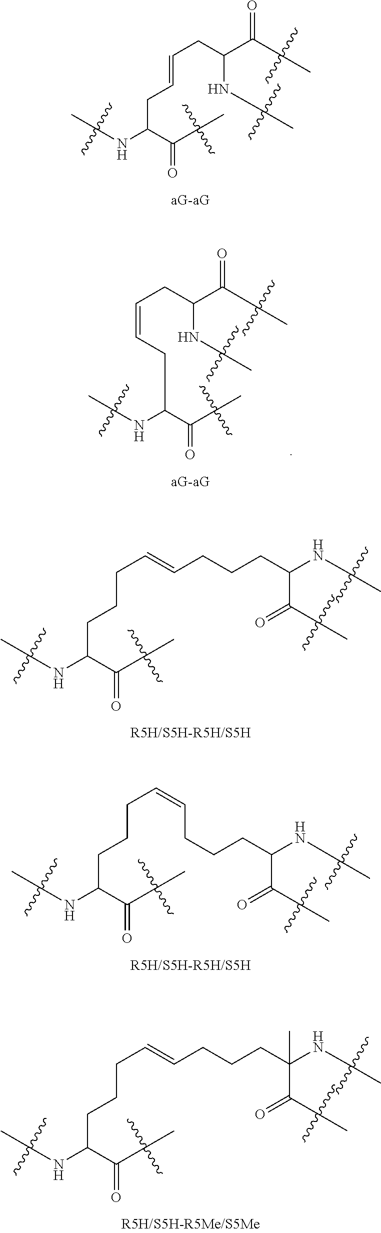 [SMILES: *NC(C/C=C/CC(N*)C(*)=O)C(*)=O ; *NC(C/C=C\CC(N*)C(*)=O)C(*)=O ; *NC(CCC/C=C/CCCC(N*)C(*)=O)C(*)=O ; *NC(CCC/C=C\CCCC(N*)C(*)=O)C(*)=O ; *NC(CCC/C=C/CCCC(C)(N*)C(*)=O)C(*)=O]

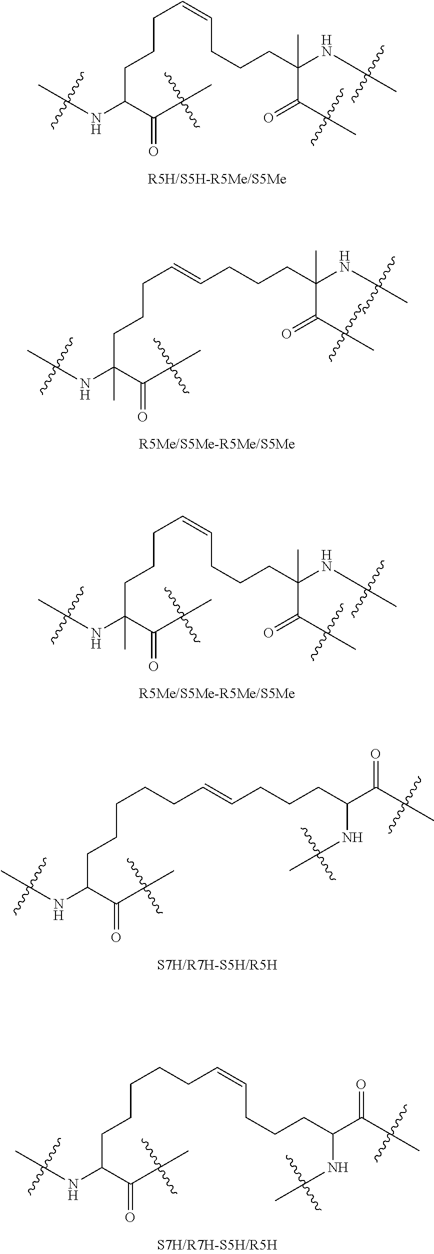 [SMILES: *NC(CCC/C=C\CCCC(C)(N*)C(*)=O)C(*)=O ; *NC(C)(CCC/C=C/CCCC(C)(N*)C(*)=O)C(*)=O ; *NC(C)(CCC/C=C\CCCC(C)(N*)C(*)=O)C(*)=O ; *NC(CCC/C=C/CCCCCC(N*)C(*)=O)C(*)=O ; *NC(CCC/C=C\CCCCCC(N*)C(*)=O)C(*)=O]

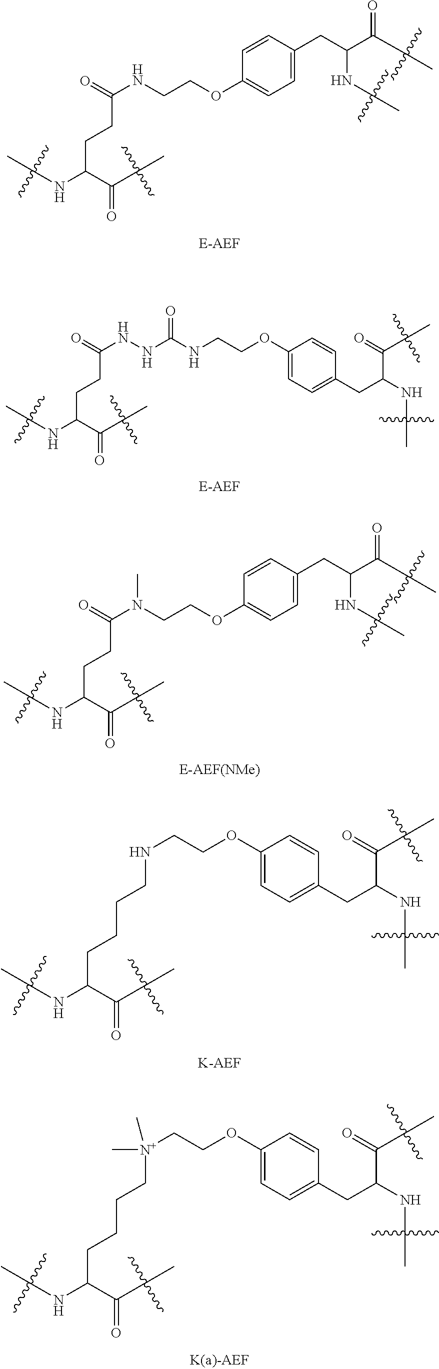 [SMILES: *NC(CCC(=O)NCCOc1ccc(CC(N*)C(*)=O)cc1)C(*)=O ; *NC(CCC(=O)NNC(=O)NCCOc1ccc(CC(N*)C(*)=O)cc1)C(*)=O ; *NC(CCC(=O)N(C)CCOc1ccc(CC(N*)C(*)=O)cc1)C(*)=O ; *NC(CCCCNCCOc1ccc(CC(N*)C(*)=O)cc1)C(*)=O ; *NC(CCCC[N+](C)(C)CCOc1ccc(CC(N*)C(*)=O)cc1)C(*)=O]

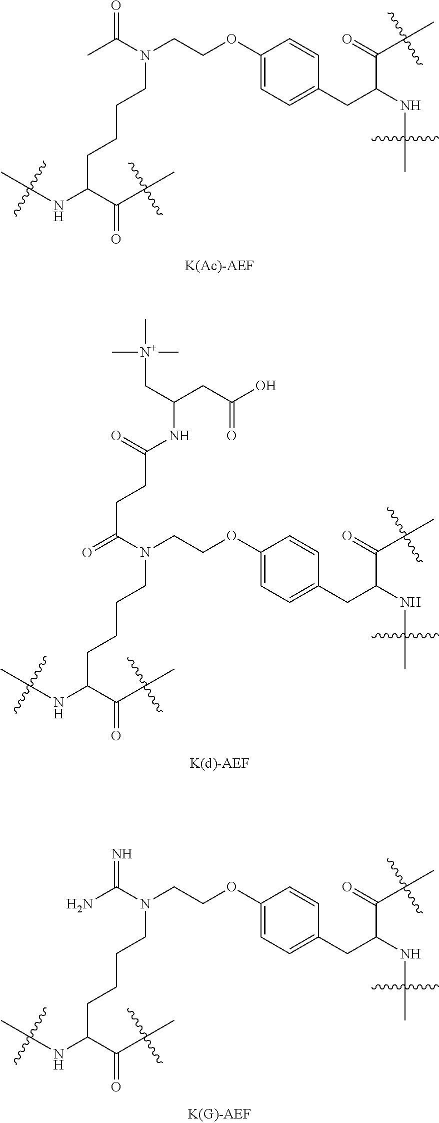 [SMILES: *NC(CCCCN(CCOc1ccc(CC(N*)C(*)=O)cc1)C(C)=O)C(*)=O ; *NC(CCCCN(CCOc1ccc(CC(N*)C(*)=O)cc1)C(=O)CCC(=O)NC(CC(=O)O)C[N+](C)(C)C)C(*)=O ; *NC(CCCCN(CCOc1ccc(CC(N*)C(*)=O)cc1)C(=N)N)C(*)=O]

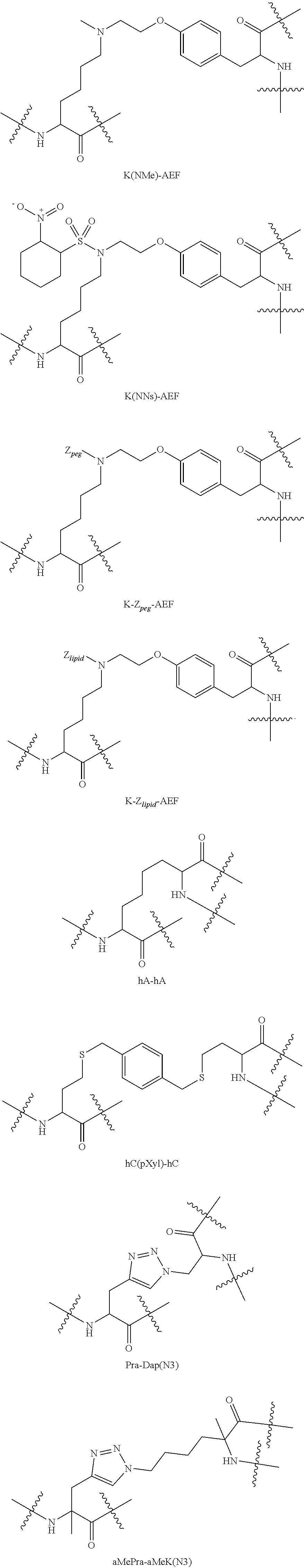

In some embodiments, the second ring comprises a linkage between the N-terminus of the peptide and a ring-forming amino acid and has a structure selected from the following:

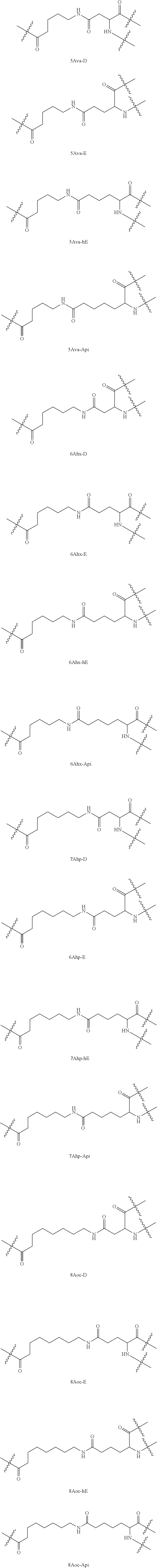

In some embodiments, the second ring is formed between X₄ and X₉, X₄ and X₁₃, X₅ and X₁₀, X₃ and X₁₃, or X₆ and X₉. In some embodiments, the second ring is formed between X₄ and X₉, X₄ and X₁₃, X₅ and X₁₀, X₃ and X₁₃, or X₆ and X₉ via a linker having one or more groups selected from the group consisting of a disulfide, thioether, amide, olefin, ether, alkylene, and triazole. In some embodiments, the second ring is formed between X₄ and X₉, X₄ and X₁₃, X₅ and X₁₀, X₃ and X₁₃, or X₆ and X₉ via a linker selected from the group consisting of a disulfide, thioether, amide, olefin, and triazole.

In some embodiments, the second ring is formed between X₅ and X₁₀ or X₃ and X₁₃. In some embodiments, the second ring is formed between X₅ and X₁₀ or X₃ and X₁₃ via a linker having one or more groups selected from the group consisting of a disulfide, thioether, amide, olefin, ether, alkylene, and triazole. In some embodiments, the second ring is formed between X₅ and X₁₀ or X₃ and X₁₃ via a linker having one or more groups selected from the group consisting of a disulfide, thioether, amide, olefin, and triazole. In some embodiments, the second ring is formed between X₅ and X₁₀ or X₃ and X₁₃ via a linker selected from the group consisting of a disulfide, thioether, amide, olefin, and triazole.

In some embodiments, the second ring is formed between X₄ and X₉. In some embodiments, the second ring is formed between X₄ and X₉ via a linker having one or more groups selected from the group consisting of a disulfide, thioether, amide, olefin, ether, alkylene, and triazole. In some embodiments, the second ring is formed between X₄ and X₉ via a linker selected from the group consisting of a disulfide, thioether, amide, olefin, and triazole.

In some embodiments, the second ring is formed between X₄ and X₁₃. In some embodiments, the second ring is formed between X₄ and X₁₃ via a linker having one or more groups selected from the group consisting of a disulfide, thioether, amide, olefin, ether, alkylene, and triazole. In some embodiments, the second ring is formed between X₄ and X₁₃ via a linker selected from the group consisting of a disulfide, thioether, amide, olefin, and triazole.

In some embodiments, the second ring is formed between X₅ and X₁₀. In some embodiments, the second ring is formed between X₅ and X₁₀ via a linker having one or more groups selected from the group consisting of a disulfide, thioether, amide, olefin, ether, alkylene, and triazole. In some embodiments, the second ring is formed between X₅ and X₁₀ via a linker selected from the group consisting of a disulfide, thioether, amide, olefin, and triazole.

In some embodiments, the second ring is formed between X₃ and X₁₃. In some embodiments, the second ring is formed between X₃ and X₁₃ via a linker having one or more groups selected from the group consisting of a disulfide, thioether, amide, olefin, ether, alkylene, and triazole. In some embodiments, the second ring is formed between X₃ and X₁₃ via a linker selected from the group consisting of a disulfide, thioether, amide, olefin, and triazole.

In some embodiments, the second ring is formed between X₆ and X₉. In some embodiments, the second ring is formed between X₆ and X₉ via a linker having one or more groups selected from the group consisting of a disulfide, thioether, amide, olefin, ether, alkylene, and triazole. In some embodiments, the second ring is formed between X₆ and X₉ via a linker selected from the group consisting of a disulfide, thioether, amide, olefin, and triazole.

In some embodiments, the second ring is formed between X₁₃ and the N-terminus of the peptide. In some embodiments, the second ring is formed between X₁₃ and the N-terminus of the peptide via a linker having one or more groups selected from the group consisting of a disulfide, thioether, amide, olefin, ether, alkylene, and triazole. In some embodiments, the second ring is formed between X₁₃ and the N-terminus of the peptide via a linker selected from the group consisting of a disulfide, thioether, amide, olefin, and triazole.

In some embodiments, the ring is formed between X₄ and X₉ via a linker having one or more groups selected from the group consisting of a disulfide, thioether, amide, olefin, ether, alkylene, and triazole; and the second ring is formed between X₃ and X₁₃, between X₅ and X₁₀, between X₁₀ and X₁₃, or between X₁₃ and the N-terminus of the peptide via a linker having one or more groups selected from the group consisting of a disulfide, thioether, amide, olefin, ether, alkylene, and triazole.

In some embodiments, the ring is formed between X₄ and X₉ via a linker selected from the group consisting of a disulfide, thioether, amide, olefin, and triazole; and the second ring is formed between X₃ and X₁₃, between X₅ and X₁₀, between X₁₀ and X₁₃, or between X₁₃ and the N-terminus of the peptide via a linker selected from the group consisting of a disulfide, thioether, amide, olefin, and triazole.

In some embodiments, the ring is formed between X₄ and X₁₃ via a linker having one or more groups selected from the group consisting of a disulfide, thioether, amide, olefin, ether, alkylene, and triazole; and the second ring is formed between X₅ and X₁₀ or between X₆ and X₉ via a linker having one or more groups selected from the group consisting of a disulfide, thioether, amide, olefin, ether, alkylene, and triazole.

In some embodiments, the ring is formed between X₄ and X₁₃ via a linker selected from the group consisting of a disulfide, thioether, amide, olefin, and triazole; and the second ring is formed between X₅ and X₁₀ or between X₆ and X₉ via a linker selected from the group consisting of a disulfide, thioether, amide, olefin, and triazole.

In some embodiments, the ring is formed between X₆ and X₉ via a linker having one or more groups selected from the group consisting of a disulfide, thioether, amide, olefin, ether, alkylene, and triazole; and the second ring is formed between X₃ and X₁₃, between X₄ and X₁₃, between X₅ and X₁₀, between X₁₀ and X₁₃, or between X₁₃ and the N-terminus of the peptide via a linker having one or more groups selected from the group consisting of a disulfide, thioether, amide, olefin, ether, alkylene, and triazole.

In some embodiments, the ring is formed between X₆ and X₉ via a linker selected from the group consisting of a disulfide, thioether, amide, olefin, and triazole; and the second ring is formed between X₃ and X₁₃, between X₄ and X₁₃, between X₅ and X₁₀, between X₁₀ and X₁₃, or between X₁₃ and the N-terminus of the peptide via a linker selected from the group consisting of a disulfide, thioether, amide, olefin, and triazole.

In some embodiments, the peptide comprises at least one polyethylene glycol chain. In some embodiments, the peptide comprises no more than five, no more than four, no more than three, no more than two, or no more than one polyethylene glycol chain.

In some embodiments, each polyethylene glycol chain, independently, terminates in an ammonium group of a methyl group. In some embodiments, the polyethylene glycol chain terminates in an ammonium group. In some embodiments, the polyethylene glycol chain terminates in a methyl group.

In some embodiments, the peptide comprises a polyethylene glycol chain having the following structure:

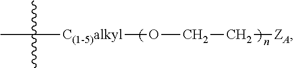

wherein:
Z_{A} is —OCH₃, —N⁺(CH₃)₃,

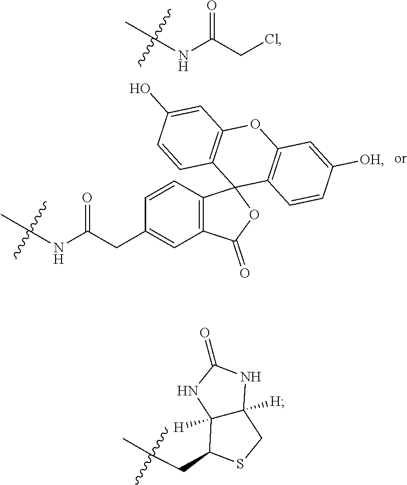

and
n is an integer from 2 to 24.

In some embodiments, the peptide comprises a polyethylene glycol chain having the following structure:

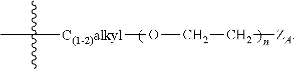

In some embodiments, the peptide comprises a polyethylene glycol chain having the following structure:

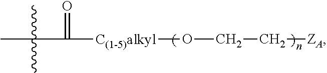

wherein:
Z_{A} is —N⁺(CH₃)₃; and
n is an integer from 2 to 24.

In some embodiments, n is an integer from 2 to 15. In some embodiments, n is an integer from 2 to 5. 2. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 11. In some embodiments, n is 12. In some embodiments, n is 14. In some embodiments, n is 14. In some embodiments, n is 15.

In some embodiments, R₁, R₂, or any amino acid of the peptide is conjugated to a polyethylene glycol chain.

Non-limiting examples of polyethylene glycol chains are provided in Table 2.

TABLE 2
Example polyethylene glycol chains

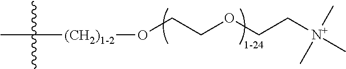

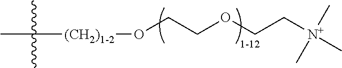

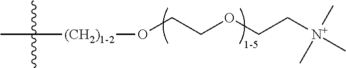

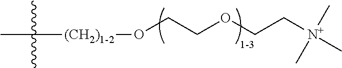

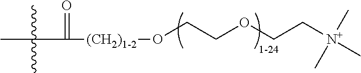

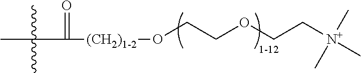

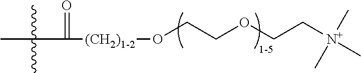

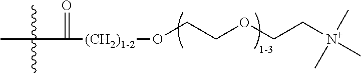

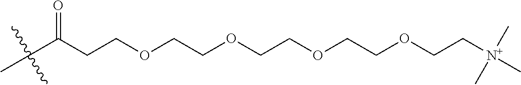 [SMILES: *C(=O)CCOCCOCCOCCOCC[N+](C)(C)C]

cPEG5a, cPEG5aCO

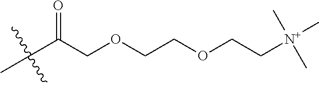 [SMILES: *C(=O)COCCOCC[N+](C)(C)C]

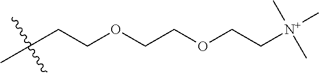 [SMILES: *CCOCCOCC[N+](C)(C)C]

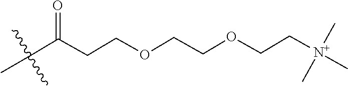 [SMILES: *C(=O)CCOCCOCC[N+](C)(C)C]

cPEG3a, cPEG3aCO

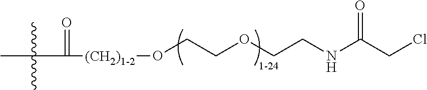

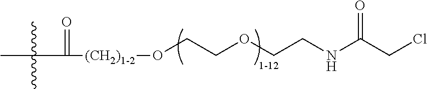

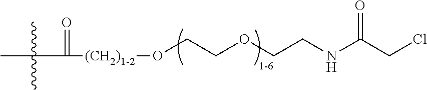

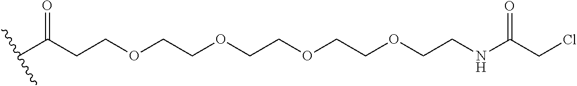 [SMILES: *C(=O)CCOCCOCCOCCOCCNC(=O)CCl]

PEG4AcCl

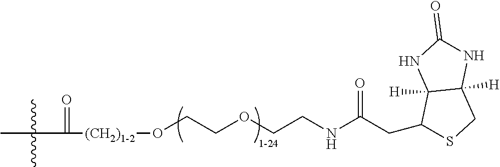

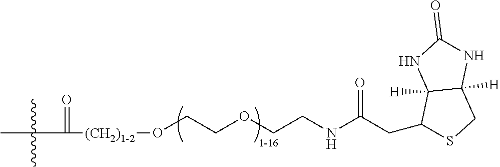

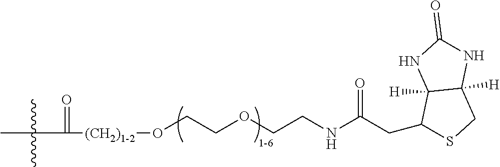

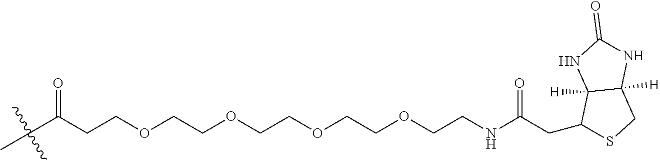 [SMILES: *C(=O)CCOCCOCCOCCOCCNC(=O)CC1SC[C@@H]2NC(=O)N[C@H]12]

BiotinPEG4

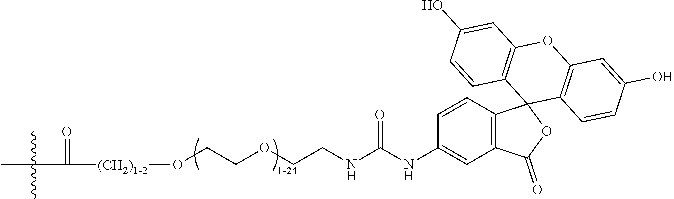

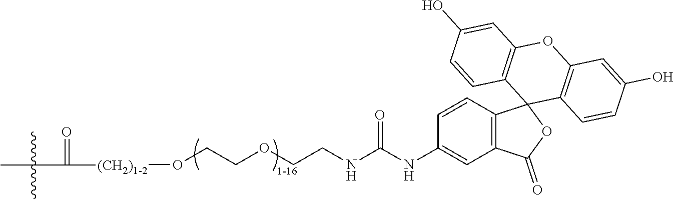

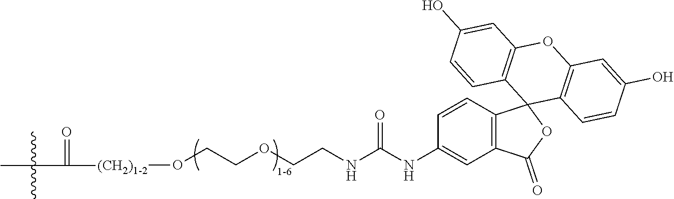

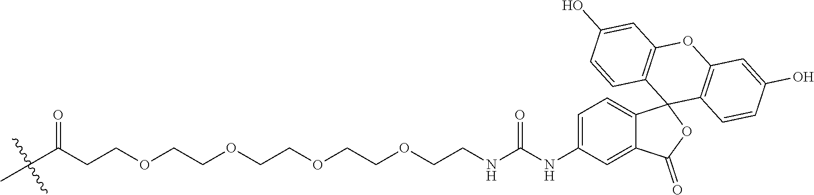 [SMILES: *C(=O)CCOCCOCCOCCOCCNC(=O)Nc1ccc2c(c1)C(=O)OC21c2ccc(O)cc2Oc2cc(O)ccc21]

FITCPEG4

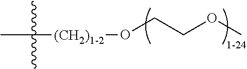

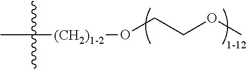

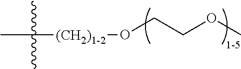

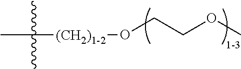

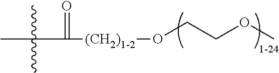

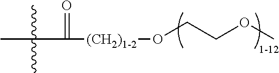

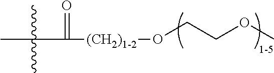

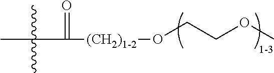

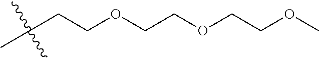 [SMILES: *CCOCCOCCOC]

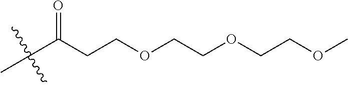 [SMILES: *C(=O)CCOCCOCCOC]

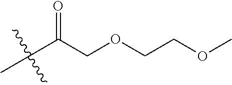 [SMILES: *C(=O)COCCOC]

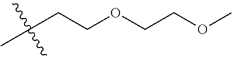 [SMILES: *CCOCCOC]

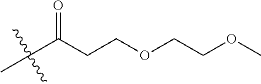 [SMILES: *C(=O)CCOCCOC]

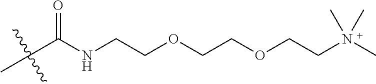

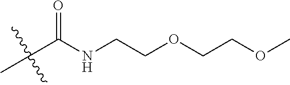 [SMILES: *C(=O)NCCOCCOC]

In some embodiments, the peptide comprises at least one lipophilic substituent. In some embodiments, the peptide comprises one lipophilic substituent. In some embodiments, the peptide comprises no more than three, no more than two, or no more than one lipophilic substituent. In some embodiments, the peptide comprises no more than one lipophilic substituent.

In some embodiments, the peptide comprises a lipophilic substituent having the following structure:

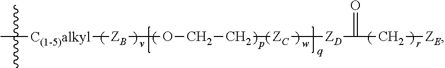

wherein:
Z_{B} is

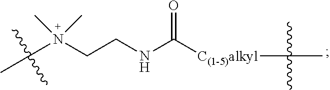

Z_{C} is Z_{C1}, Z_{C2}, or Z_{C3};
Z_{C1} is

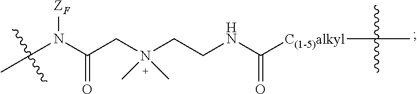

Z_{C2} is

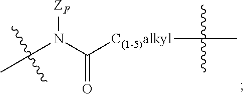

Z_{C3} is

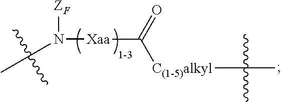

Z_{D} is

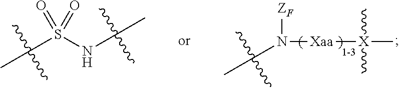

Z_{E} is —H, —COOH, or tetrazolyl;
Z_{F} is —H or —CH₃;
Xaa is, independently for each occurrence,

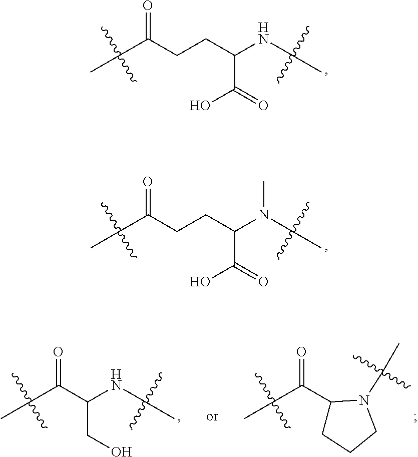 [SMILES: *NC(CCC(*)=O)C(=O)O ; *C(=O)CCC(C(=O)O)N(*)C ; *NC(CO)C(*)=O ; *C(=O)C1CCCN1*]

p, independently for each occurrence, is 0, 1, 2, 3, 4, 5, or 6;
q is 1, 2, 3, 4, 5, or 6;
r is an integer from 6 to 24;
v is 0 or 1; and
w, independently for each occurrence, is 0 or 1.

In some embodiments, the peptide comprises a lipophilic substituent having the following structure:

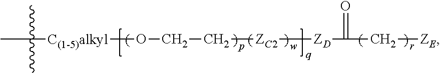

wherein:
Z_{C2} is

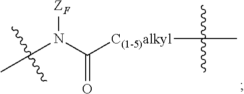

Z_{D} is

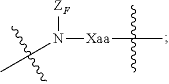

Z_{E} is —H, —COOH, or tetrazolyl;
Z_{F} is —H or —CH₃;
Xaa is

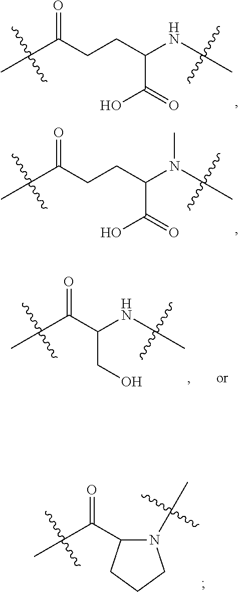

p, independently for each occurrence, is 1, 2, 3, 4, 5, or 6;
q is 1, 2, 3, 4, 5, or 6;
r is an integer from 6 to 24; and
w, independently for each occurrence, is 0 or 1.

In some embodiments, the peptide comprises a lipophilic substituent having the following structure:

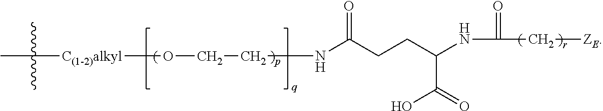

In some embodiments, the peptide comprises a lipophilic substituent having the following structure:

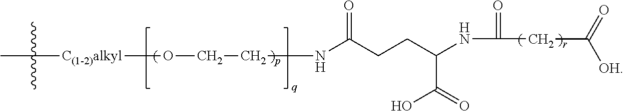

In some embodiments, the peptide comprises a lipophilic substituent having the following structure:

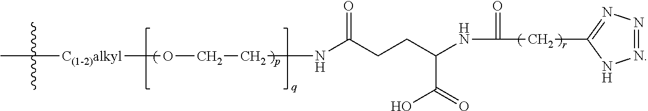

In some embodiments, the peptide comprises a lipophilic substituent having the following structure:

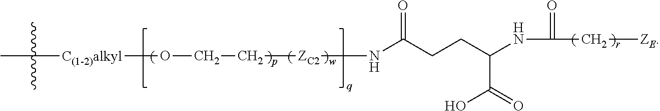

In some embodiments, the peptide comprises a lipophilic substituent having the following structure:

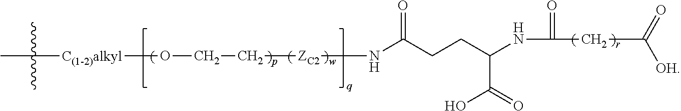

In some embodiments, the peptide comprises a lipophilic substituent having the following structure:

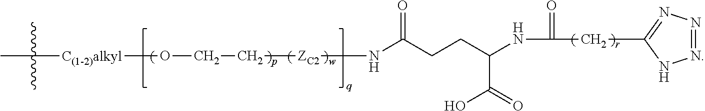

In some embodiments, the peptide comprises a lipophilic substituent having the following structure:

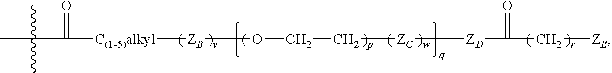

wherein:
Z_{B} is

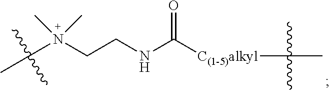

Z_{C} is Z_{C1}, Z_{C2}, or Z_{C3};
Z_{C1} is

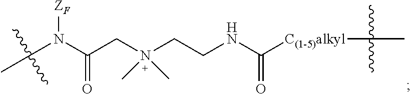

Z_{C2} is

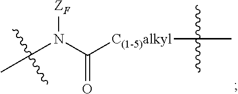

Z_{C3} is

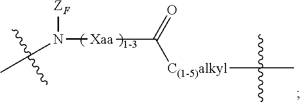

Z_{D} is

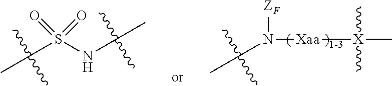

Z_{E} is —H, —COOH, or tetrazolyl;
Z_{F} is —H or —CH₃;
Xaa is, independently for each occurrence,

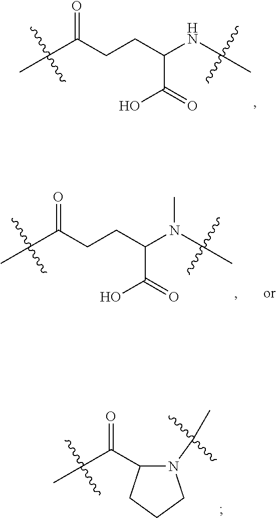 [SMILES: *NC(CCC(*)=O)C(=O)O ; *C(=O)CCC(C(=O)O)N(*)C ; *C(=O)C1CCCN1*]

p, independently for each occurrence, is 1, 2, 3, 4, 5, or 6;
q is 1, 2, 3, 4, 5, or 6;
r is an integer from 6 to 24;
v is 0 or 1; and
w, independently for each occurrence, is 0 or 1.

In some embodiments, the peptide comprises a lipophilic substituent having the following structure:

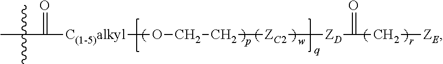

wherein:
Z_{C2} is

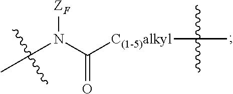

Z_{D} is

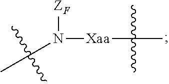

Z_{E} is —H, —COOH, or tetrazolyl;
Z_{F} is —H or —CH₃;
Xaa is

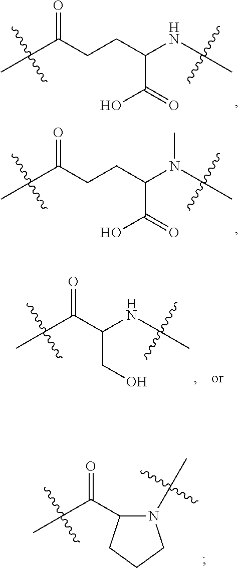

p, independently for each occurrence, is 1, 2, 3, 4, 5, or 6;
q is 1, 2, 3, 4, 5, or 6;
r is an integer from 6 to 24; and
w, independently for each occurrence, is 0 or 1.

In some embodiments, the peptide comprises a lipophilic substituent having the following structure:

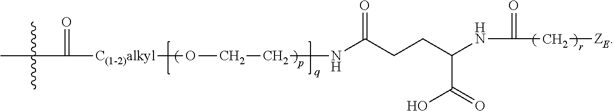

In some embodiments, the peptide comprises a lipophilic substituent having the following structure:

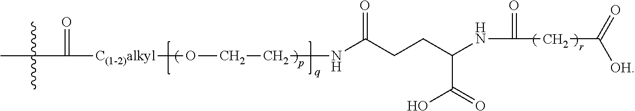

In some embodiments, the peptide comprises a lipophilic substituent having the following structure:

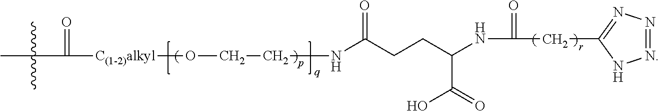

In some embodiments, the peptide comprises a lipophilic substituent having the following structure:

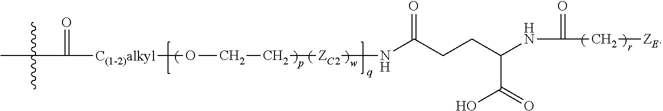

In some embodiments, the peptide comprises a lipophilic substituent having the following structure:

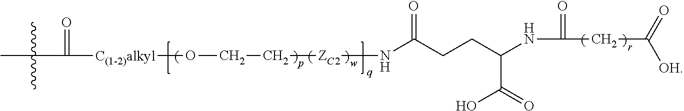

In some embodiments, the peptide comprises a lipophilic substituent having the following structure:

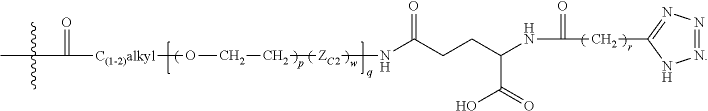

In some embodiments, Z_{c} is

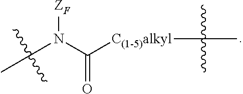

In some embodiments, Z_{D} is

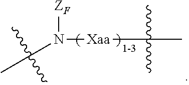

In some embodiments, Z_{D} is

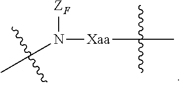

In some embodiments, Z_{E} is —H. In some embodiments, Z_{E} is —COOH. In some embodiments, Z_{E} is tetrazolyl.

In some embodiments, Z_{F} is —H. In some embodiments, Z_{F} is —CH₃.

In some embodiments, Xaa is, independently for each occurrence,

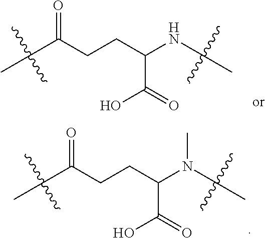

In some embodiments, Xaa is

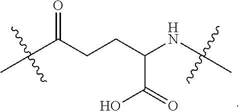 [SMILES: *NC(CCC(*)=O)C(=O)O]

In some embodiments, p is 1, 2, 3, or 4. In some embodiments, p is 1. In some embodiments, p is 2. In some embodiments, p is 3. In some embodiments, p is 4. In some embodiments, p is 5. In some embodiments, p is 6.

In some embodiments, q is 1, 2, 3, or 4. In some embodiments, q is 1. In some embodiments, q is 2. In some embodiments, q is 3. In some embodiments, q is 4. In some embodiments, q is 5. In some embodiments, q is 6.

In some embodiments, r is an integer between 10 and 20. In some embodiments, r is 10. In some embodiments, r is 11. In some embodiments, r is 12. In some embodiments, r is 13. In some embodiments, r is 14. In some embodiments, r is 15. In some embodiments, r is 16. In some embodiments, r is 17. In some embodiments, r is 18. In some embodiments, r is 19. In some embodiments, r is 20.

In some embodiments, w is 0. In some embodiments, w is 1.

In some embodiments, R₁, R₂, or any amino acid of the peptide is conjugated to a lipophilic substituent.

Non-limiting examples of lipophilic substituents are provided in Table 3A.

TABLE 3A
Example lipophilic substituents

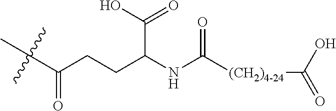 [SMILES: *C(=O)CCC(NC(=O)CC(=O)O)C(=O)O]

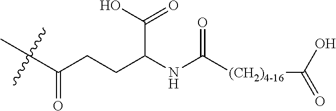 [SMILES: *C(=O)CCC(NC(=O)CC(=O)O)C(=O)O]

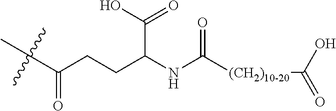 [SMILES: *C(=O)CCC(NC(=O)CC(=O)O)C(=O)O]

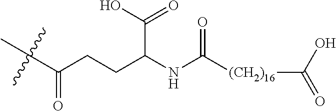

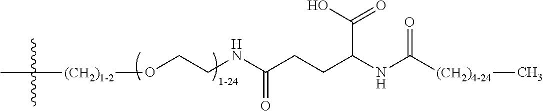

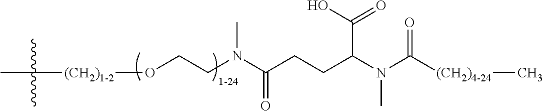

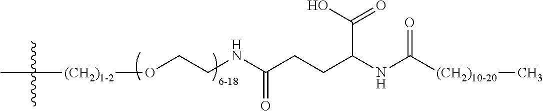

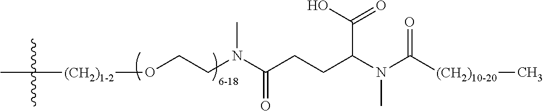

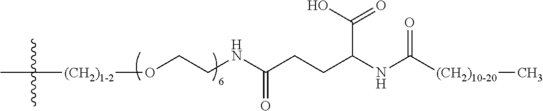

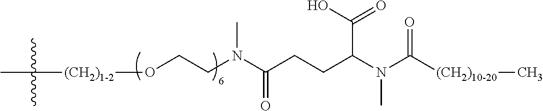

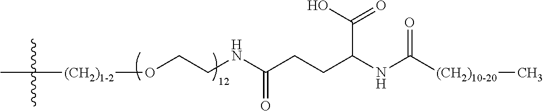

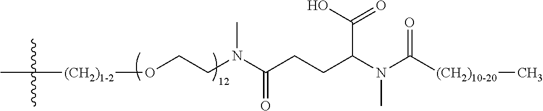

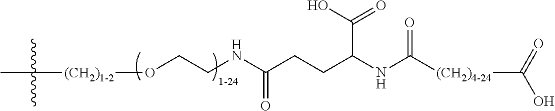

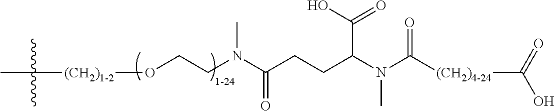

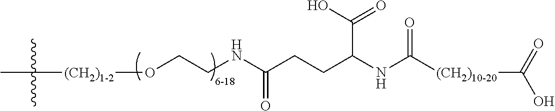

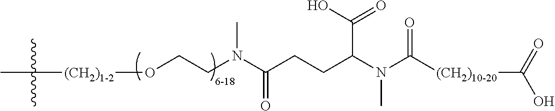

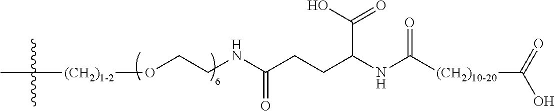

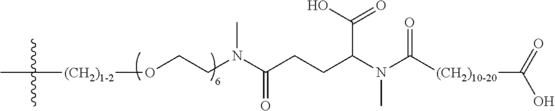

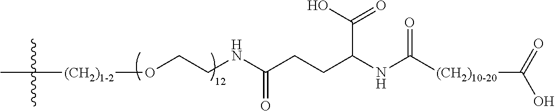

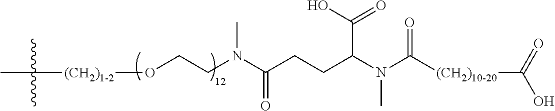

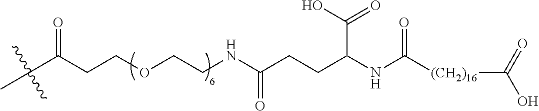

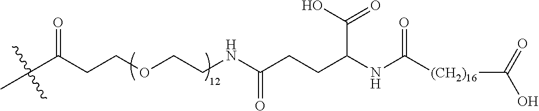

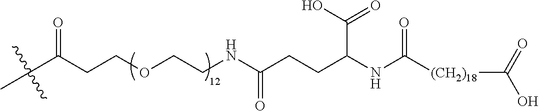

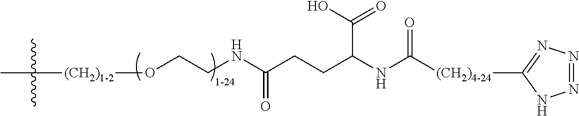

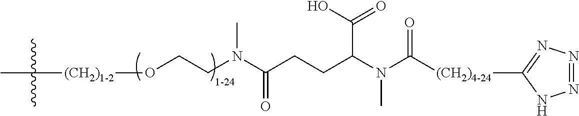

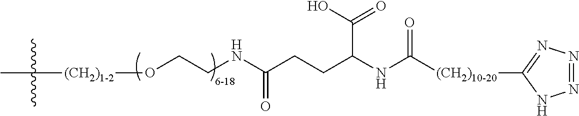

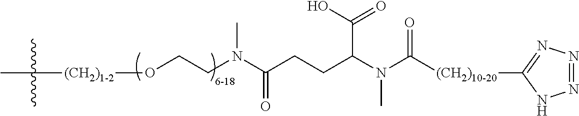

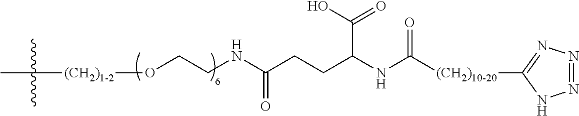

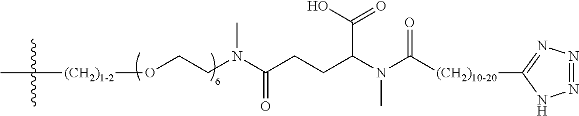

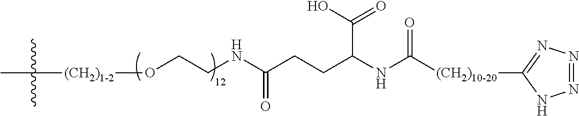

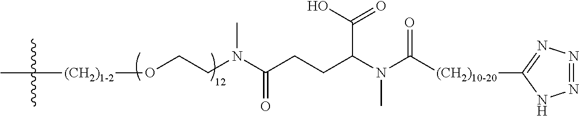

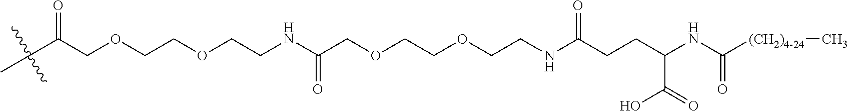 [SMILES: *C(=O)COCCOCCNC(=O)COCCOCCNC(=O)CCC(NC(=O)CC)C(=O)O]

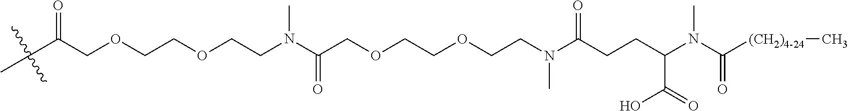 [SMILES: *C(=O)COCCOCCN(C)C(=O)COCCOCCN(C)C(=O)CCC(C(=O)O)N(C)C(=O)CC]

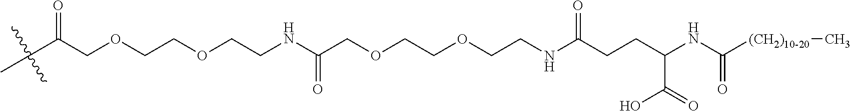 [SMILES: *C(=O)COCCOCCNC(=O)COCCOCCNC(=O)CCC(NC(=O)CC)C(=O)O]

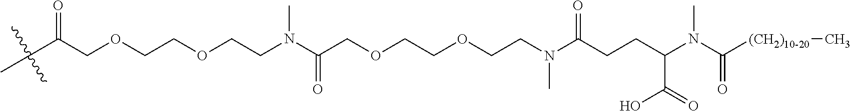 [SMILES: *C(=O)COCCOCCN(C)C(=O)COCCOCCN(C)C(=O)CCC(C(=O)O)N(C)C(=O)CC]

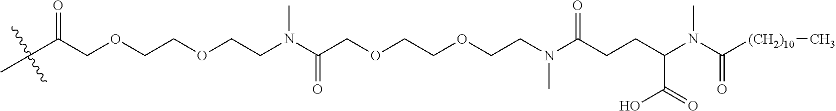

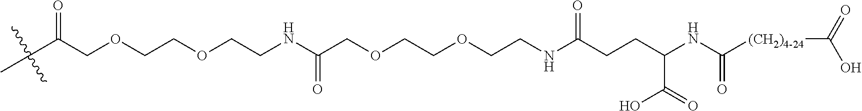 [SMILES: *C(=O)COCCOCCNC(=O)COCCOCCNC(=O)CCC(NC(=O)CC(=O)O)C(=O)O]

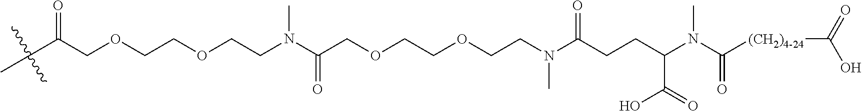 [SMILES: *C(=O)COCCOCCN(C)C(=O)COCCOCCN(C)C(=O)CCC(C(=O)O)N(C)C(=O)CC(=O)O]

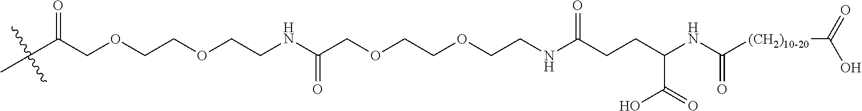 [SMILES: *C(=O)COCCOCCNC(=O)COCCOCCNC(=O)CCC(NC(=O)CC(=O)O)C(=O)O]

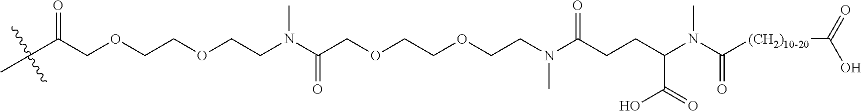 [SMILES: *C(=O)COCCOCCN(C)C(=O)COCCOCCN(C)C(=O)CCC(C(=O)O)N(C)C(=O)CC(=O)O]

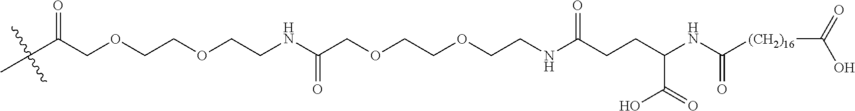

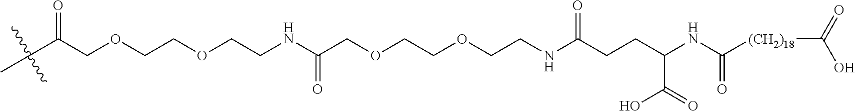

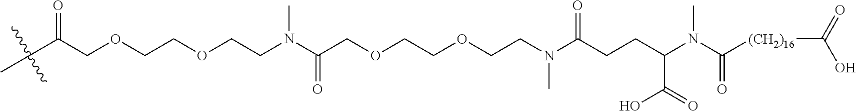

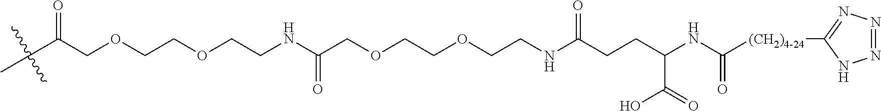 [SMILES: *C(=O)COCCOCCNC(=O)COCCOCCNC(=O)CCC(NC(=O)Cc1nnn[nH]1)C(=O)O]

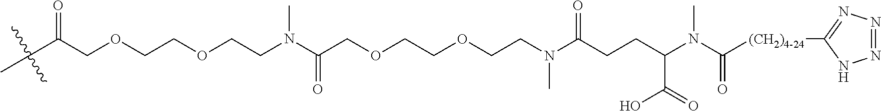 [SMILES: *C(=O)COCCOCCN(C)C(=O)COCCOCCN(C)C(=O)CCC(C(=O)O)N(C)C(=O)Cc1nnn[nH]1]

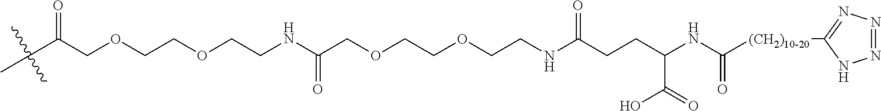 [SMILES: *C(=O)COCCOCCNC(=O)COCCOCCNC(=O)CCC(NC(=O)Cc1nnn[nH]1)C(=O)O]

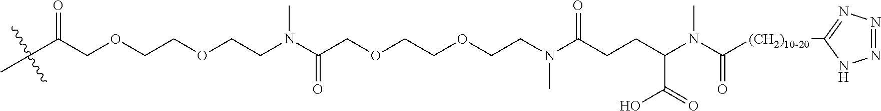 [SMILES: *C(=O)COCCOCCN(C)C(=O)COCCOCCN(C)C(=O)CCC(C(=O)O)N(C)C(=O)Cc1nnn[nH]1]

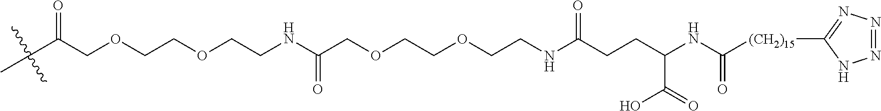

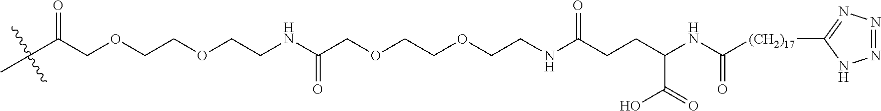

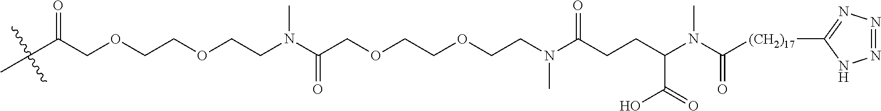

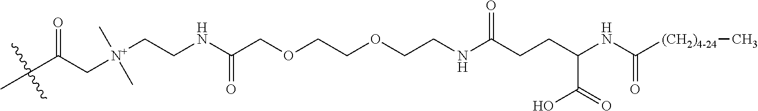 [SMILES: *C(=O)C[N+](C)(C)CCNC(=O)COCCOCCNC(=O)CCC(NC(=O)CC)C(=O)O]

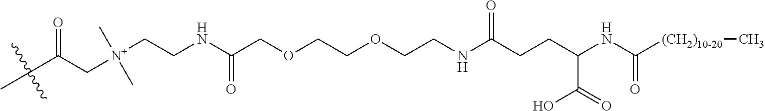 [SMILES: *C(=O)C[N+](C)(C)CCNC(=O)COCCOCCNC(=O)CCC(NC(=O)CC)C(=O)O]

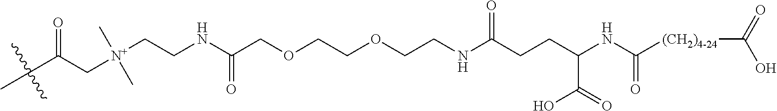 [SMILES: *C(=O)C[N+](C)(C)CCNC(=O)COCCOCCNC(=O)CCC(NC(=O)CC(=O)O)C(=O)O]

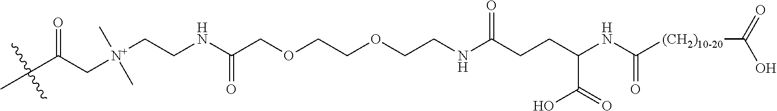 [SMILES: *C(=O)C[N+](C)(C)CCNC(=O)COCCOCCNC(=O)CCC(NC(=O)CC(=O)O)C(=O)O]

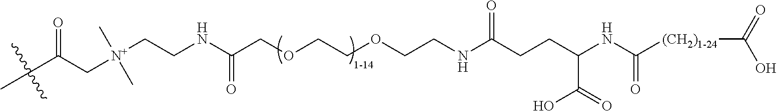

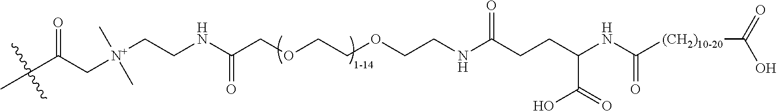

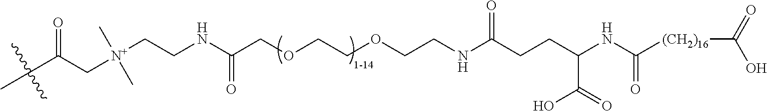

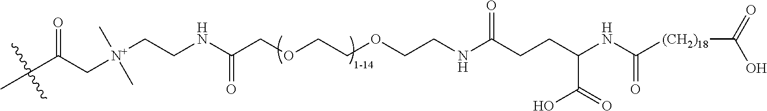

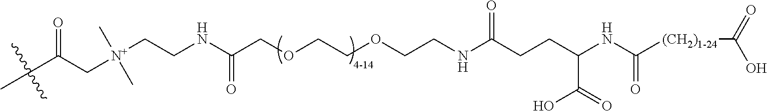

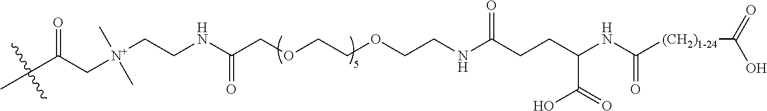

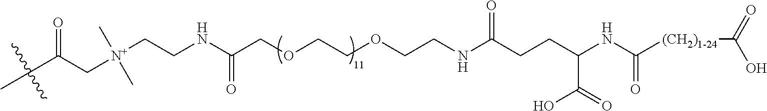

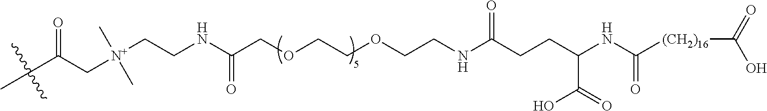

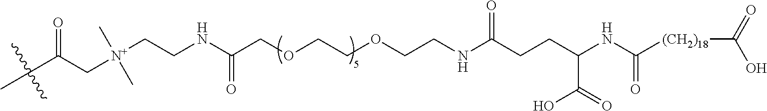

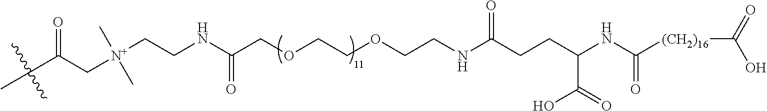

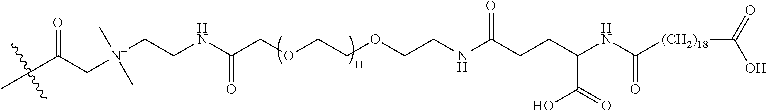

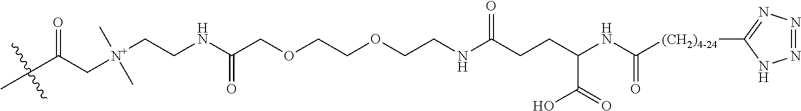 [SMILES: *C(=O)C[N+](C)(C)CCNC(=O)COCCOCCNC(=O)CCC(NC(=O)Cc1nnn[nH]1)C(=O)O]

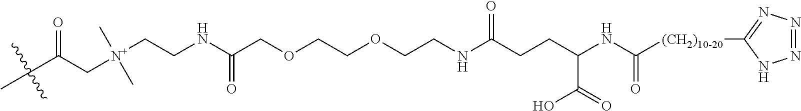 [SMILES: *C(=O)C[N+](C)(C)CCNC(=O)COCCOCCNC(=O)CCC(NC(=O)Cc1nnn[nH]1)C(=O)O]

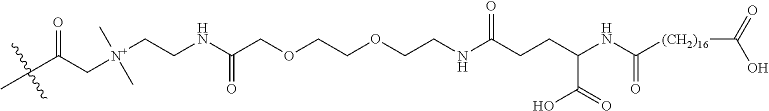

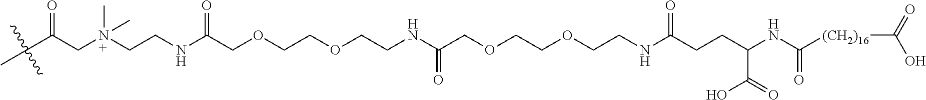

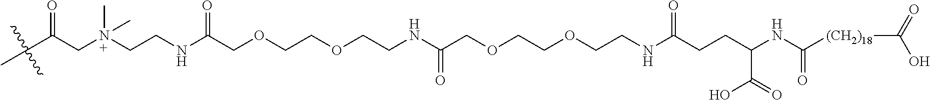

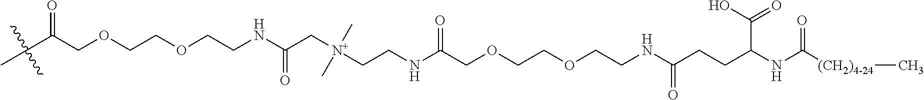 [SMILES: *C(=O)COCCOCCNC(=O)C[N+](C)(C)CCNC(=O)COCCOCCNC(=O)CCC(NC(=O)CC)C(=O)O]

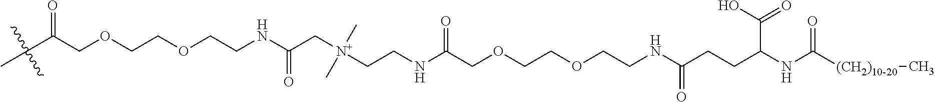 [SMILES: *C(=O)COCCOCCNC(=O)C[N+](C)(C)CCNC(=O)COCCOCCNC(=O)CCC(NC(=O)CC)C(=O)O]

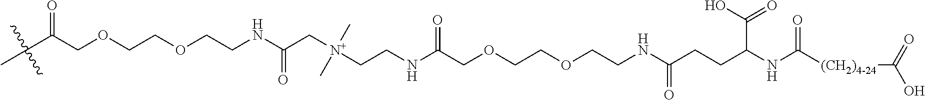 [SMILES: *C(=O)COCCOCCNC(=O)C[N+](C)(C)CCNC(=O)COCCOCCNC(=O)CCC(NC(=O)CC(=O)O)C(=O)O]

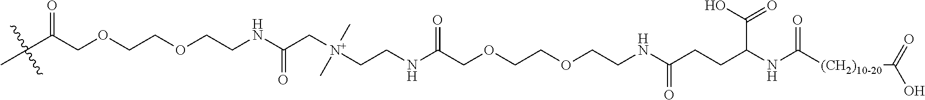 [SMILES: *C(=O)COCCOCCNC(=O)C[N+](C)(C)CCNC(=O)COCCOCCNC(=O)CCC(NC(=O)CC(=O)O)C(=O)O]

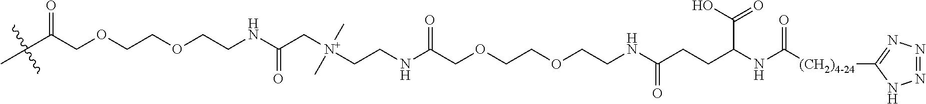 [SMILES: *C(=O)COCCOCCNC(=O)C[N+](C)(C)CCNC(=O)COCCOCCNC(=O)CCC(NC(=O)Cc1nnn[nH]1)C(=O)O]

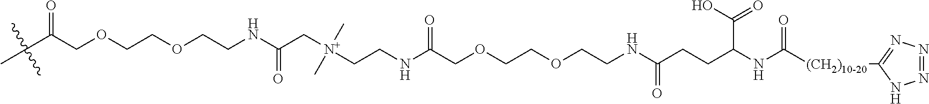 [SMILES: *C(=O)COCCOCCNC(=O)C[N+](C)(C)CCNC(=O)COCCOCCNC(=O)CCC(NC(=O)Cc1nnn[nH]1)C(=O)O]

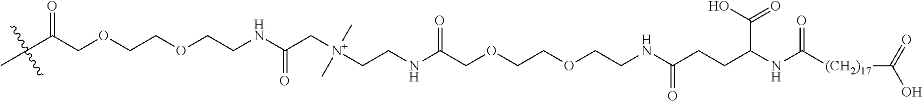

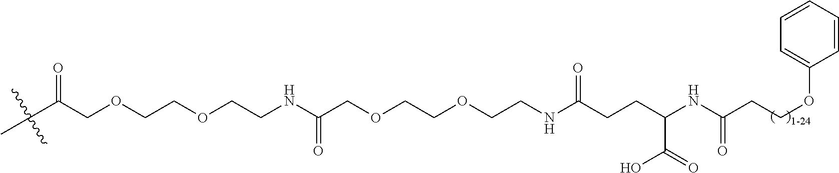

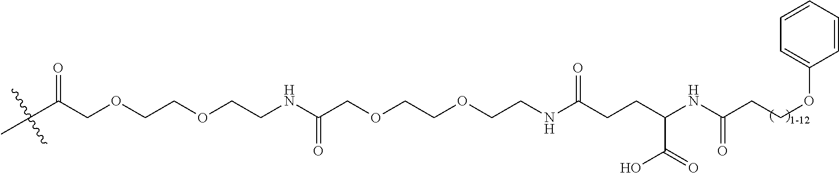

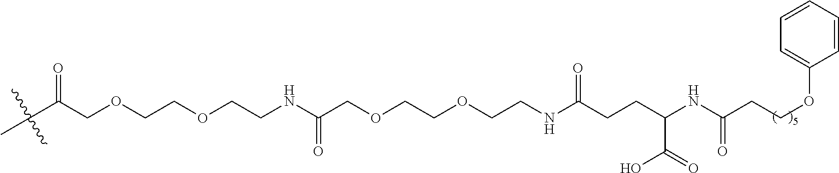

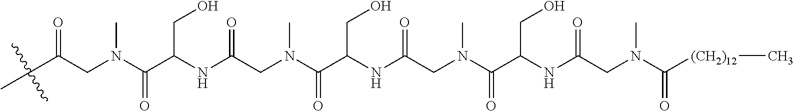

Further non-limiting examples of lipophilic substituents are provided in Table 3B.

TABLE 3B
Example lipophilic substituents

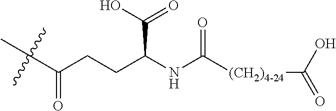 [SMILES: *C(=O)CC[C@H](NC(=O)CC(=O)O)C(=O)O]

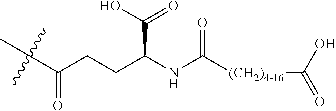 [SMILES: *C(=O)CC[C@H](NC(=O)CC(=O)O)C(=O)O]

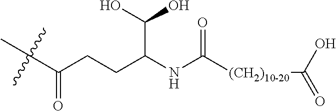

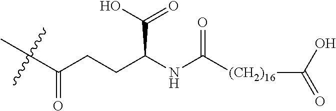

gEC18OH

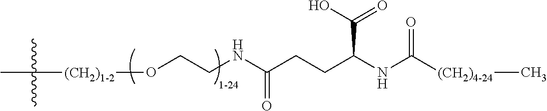

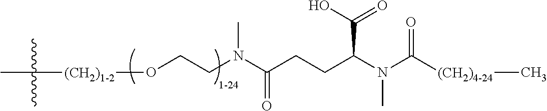

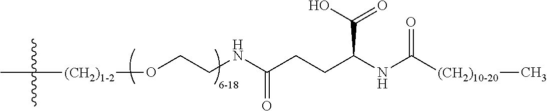

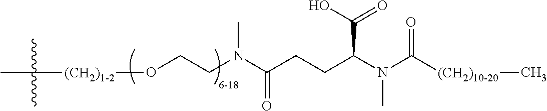

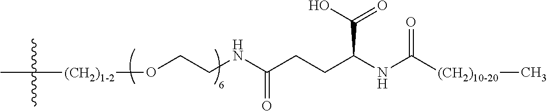

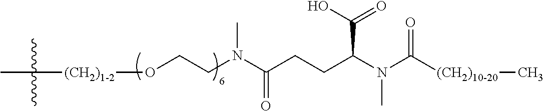

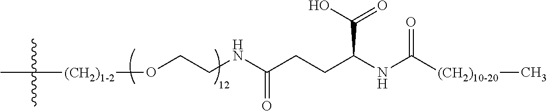

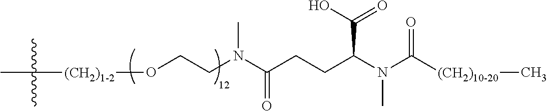

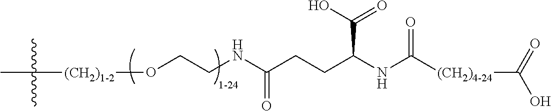

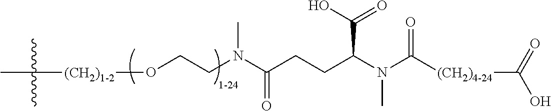

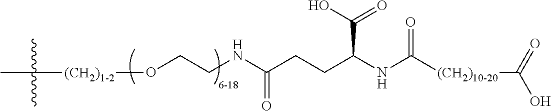

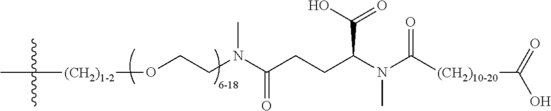

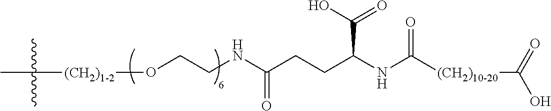

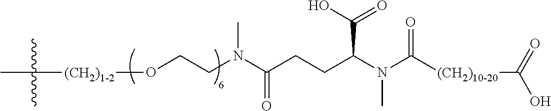

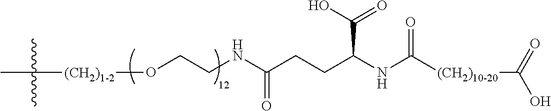

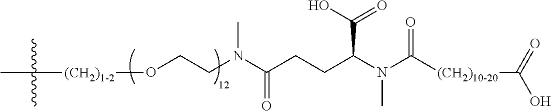

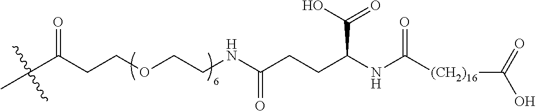

PEG6gEC18OH, HOC18gEPEG6CO

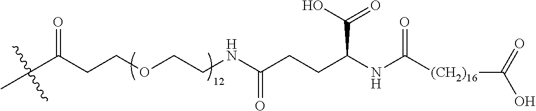

PEG12gEC18OH

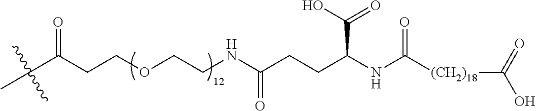

PEG12gEC20OH

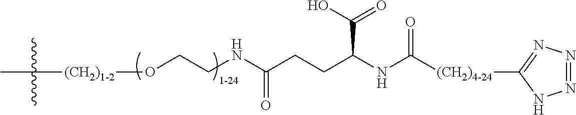

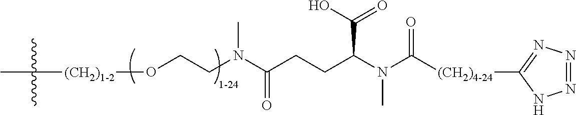

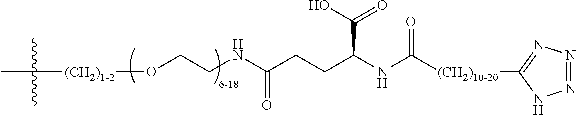

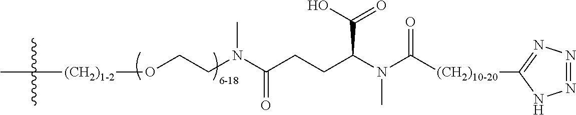

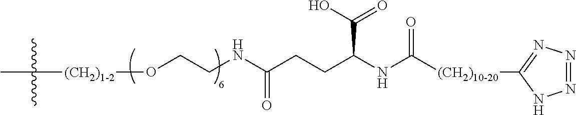

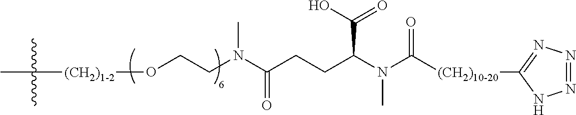

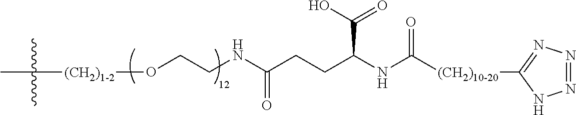

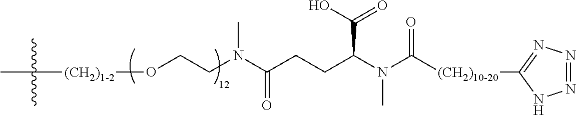

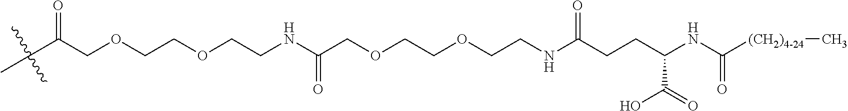 [SMILES: *C(=O)COCCOCCNC(=O)COCCOCCNC(=O)CC[C@H](NC(=O)CC)C(=O)O]

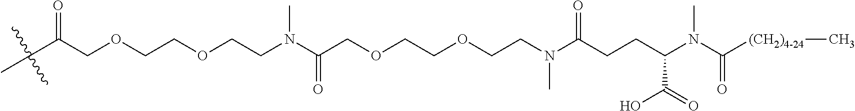 [SMILES: *C(=O)COCCOCCN(C)C(=O)COCCOCCN(C)C(=O)CC[C@@H](C(=O)O)N(C)C(=O)CC]

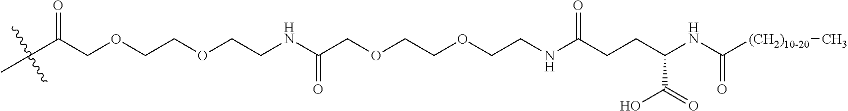 [SMILES: *C(=O)COCCOCCNC(=O)COCCOCCNC(=O)CC[C@H](NC(=O)CC)C(=O)O]

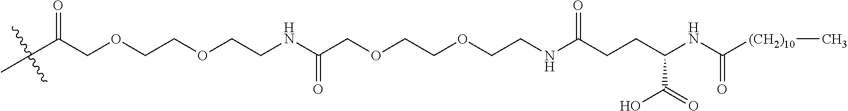

C12gEPEG2PEG2CO

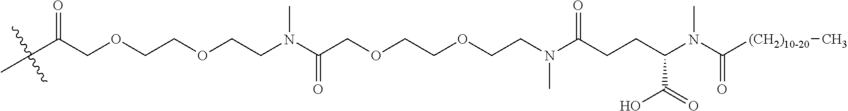 [SMILES: *C(=O)COCCOCCN(C)C(=O)COCCOCCN(C)C(=O)CC[C@@H](C(=O)O)N(C)C(=O)CC]

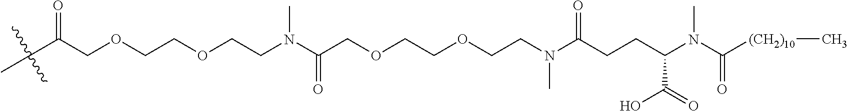

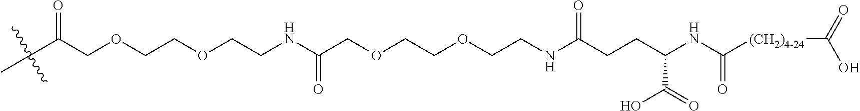 [SMILES: *C(=O)COCCOCCNC(=O)COCCOCCNC(=O)CC[C@H](NC(=O)CC(=O)O)C(=O)O]

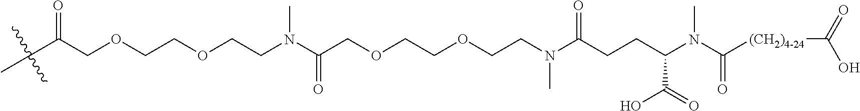 [SMILES: *C(=O)COCCOCCN(C)C(=O)COCCOCCN(C)C(=O)CC[C@@H](C(=O)O)N(C)C(=O)CC(=O)O]

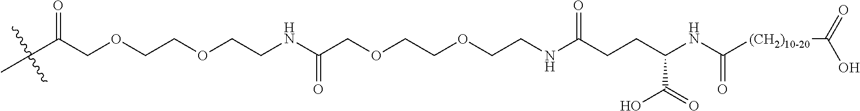 [SMILES: *C(=O)COCCOCCNC(=O)COCCOCCNC(=O)CC[C@H](NC(=O)CC(=O)O)C(=O)O]

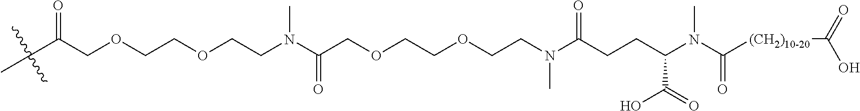 [SMILES: *C(=O)COCCOCCN(C)C(=O)COCCOCCN(C)C(=O)CC[C@@H](C(=O)O)N(C)C(=O)CC(=O)O]

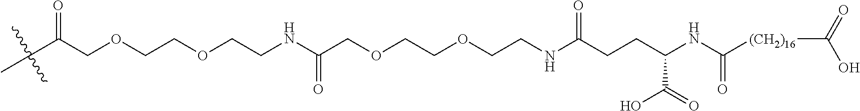

HOC18gEPEG2PEG2CO, PEG2PEG2gEC18OH

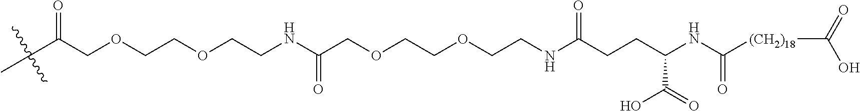

HOC20gEPEG2PEG2CO, PEG2PEG2gEC20OH

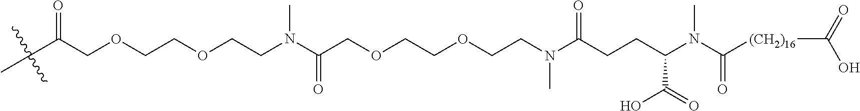

PEG2NMePEG2NMegENMeC18OH

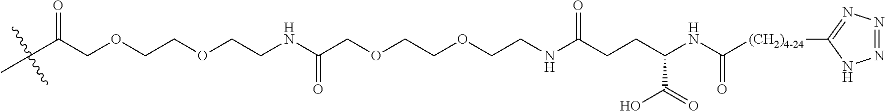 [SMILES: *C(=O)COCCOCCNC(=O)COCCOCCNC(=O)CC[C@H](NC(=O)Cc1nnn[nH]1)C(=O)O]

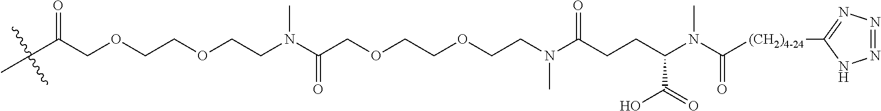 [SMILES: *C(=O)COCCOCCN(C)C(=O)COCCOCCN(C)C(=O)CC[C@@H](C(=O)O)N(C)C(=O)Cc1nnn[nH]1]

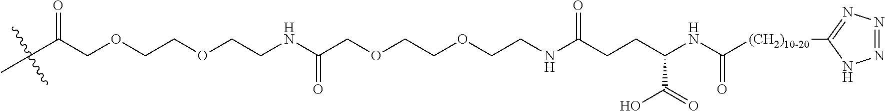 [SMILES: *C(=O)COCCOCCNC(=O)COCCOCCNC(=O)CC[C@H](NC(=O)Cc1nnn[nH]1)C(=O)O]

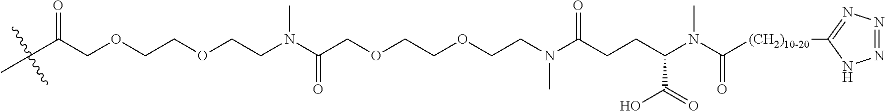 [SMILES: *C(=O)COCCOCCN(C)C(=O)COCCOCCN(C)C(=O)CC[C@@H](C(=O)O)N(C)C(=O)Cc1nnn[nH]1]

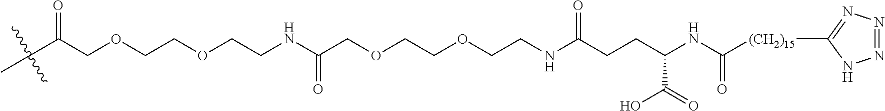

PEG2PEG2gEC16tetrazole

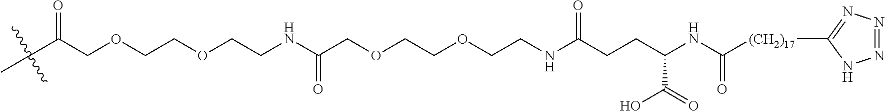

PEG2PEG2gEC18tetrazole

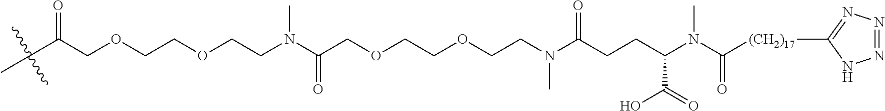

PEG2NMePEG2NMegENMeC18Tetrazole

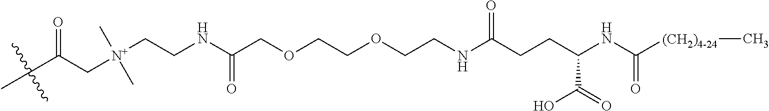 [SMILES: *C(=O)C[N+](C)(C)CCNC(=O)COCCOCCNC(=O)CC[C@H](NC(=O)CC)C(=O)O]

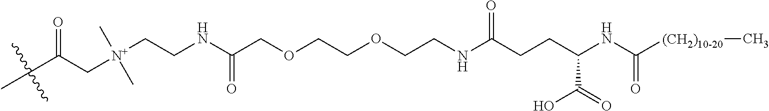 [SMILES: *C(=O)C[N+](C)(C)CCNC(=O)COCCOCCNC(=O)CC[C@H](NC(=O)CC)C(=O)O]

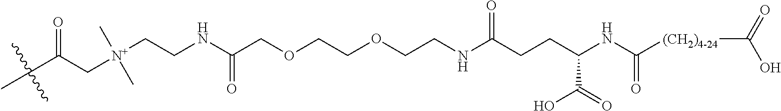 [SMILES: *C(=O)C[N+](C)(C)CCNC(=O)COCCOCCNC(=O)CC[C@H](NC(=O)CC(=O)O)C(=O)O]

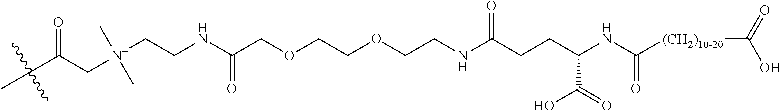 [SMILES: *C(=O)C[N+](C)(C)CCNC(=O)COCCOCCNC(=O)CC[C@H](NC(=O)CC(=O)O)C(=O)O]

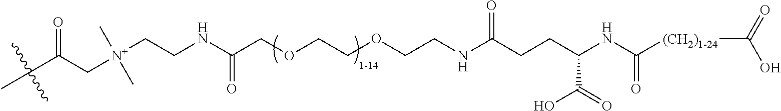

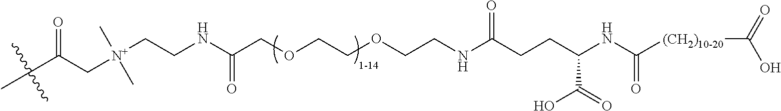

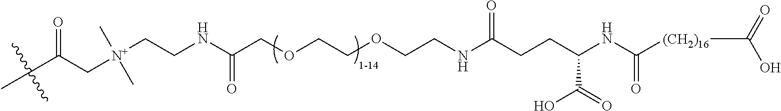

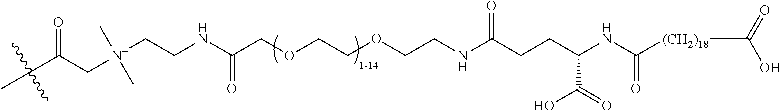

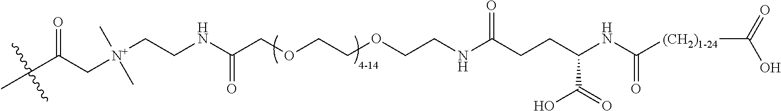

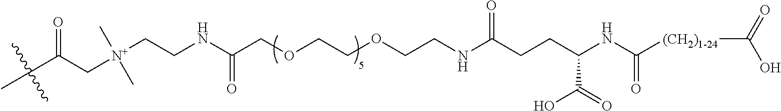

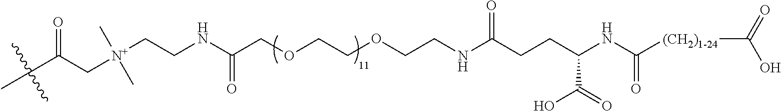

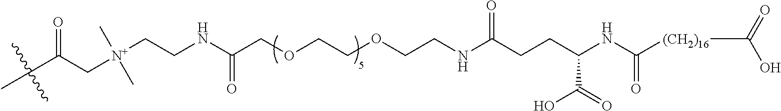

SP6PEG6gEC18OH

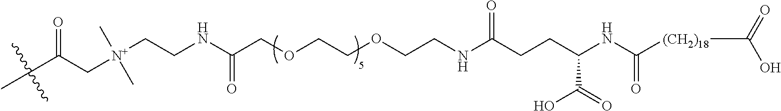

SP6PEG6gEC20OH

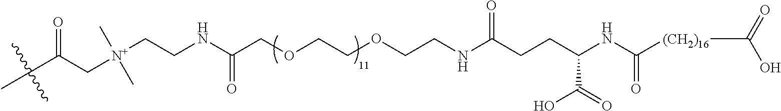

SP6PEG12gEC18OH

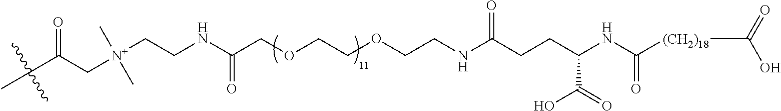

SP6PEG12gEC20OH

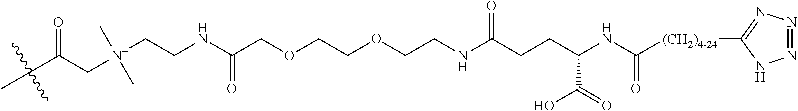 [SMILES: *C(=O)C[N+](C)(C)CCNC(=O)COCCOCCNC(=O)CC[C@H](NC(=O)Cc1nnn[nH]1)C(=O)O]

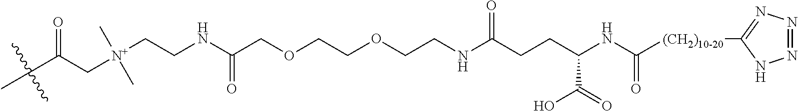 [SMILES: *C(=O)C[N+](C)(C)CCNC(=O)COCCOCCNC(=O)CC[C@H](NC(=O)Cc1nnn[nH]1)C(=O)O]

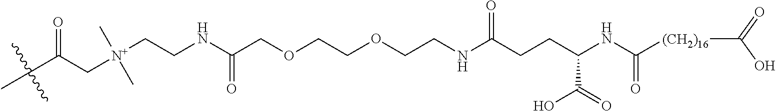

SP6PEG2gEC18OH

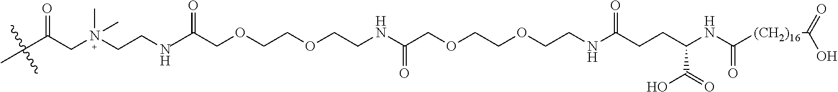

HOC18gEPEG2PEG2COSP6

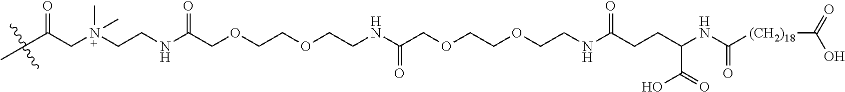

HOC20gEPEG2PEG2COSP6

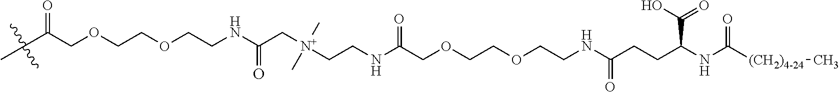 [SMILES: *C(=O)COCCOCCNC(=O)C[N+](C)(C)CCNC(=O)COCCOCCNC(=O)CC[C@H](NC(=O)CC)C(=O)O]

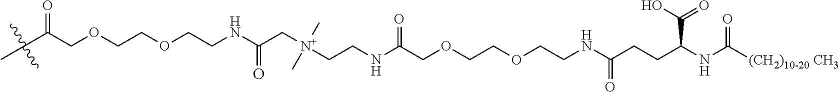 [SMILES: *C(=O)COCCOCCNC(=O)C[N+](C)(C)CCNC(=O)COCCOCCNC(=O)CC[C@H](NC(=O)CC)C(=O)O]

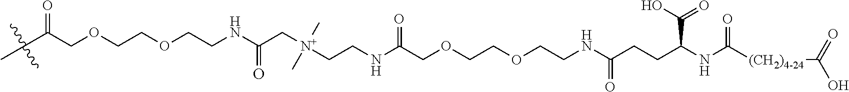 [SMILES: *C(=O)COCCOCCNC(=O)C[N+](C)(C)CCNC(=O)COCCOCCNC(=O)CC[C@H](NC(=O)CC(=O)O)C(=O)O]

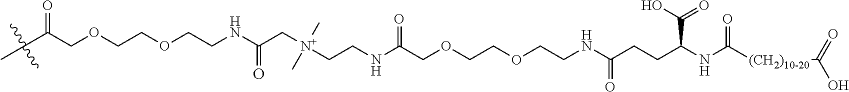 [SMILES: *C(=O)COCCOCCNC(=O)C[N+](C)(C)CCNC(=O)COCCOCCNC(=O)CC[C@H](NC(=O)CC(=O)O)C(=O)O]

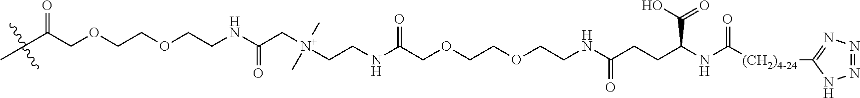 [SMILES: *C(=O)COCCOCCNC(=O)C[N+](C)(C)CCNC(=O)COCCOCCNC(=O)CC[C@H](NC(=O)Cc1nnn[nH]1)C(=O)O]

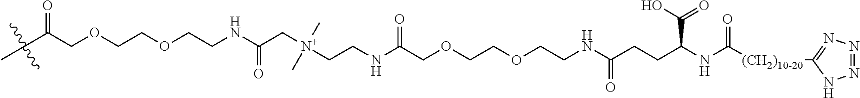 [SMILES: *C(=O)COCCOCCNC(=O)C[N+](C)(C)CCNC(=O)COCCOCCNC(=O)CC[C@H](NC(=O)Cc1nnn[nH]1)C(=O)O]

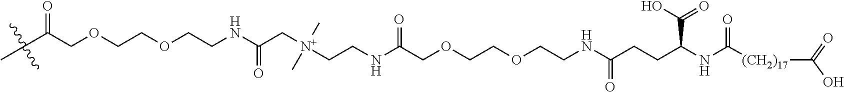

HOC18gEPEG2SP6PEG2CO

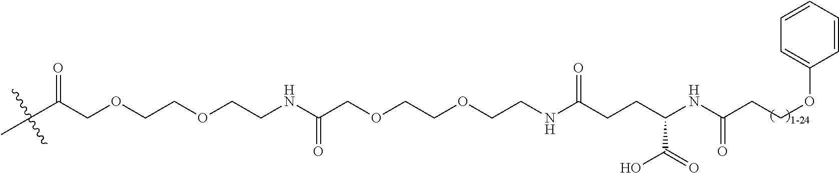

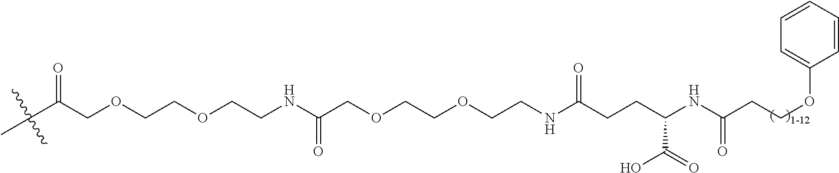

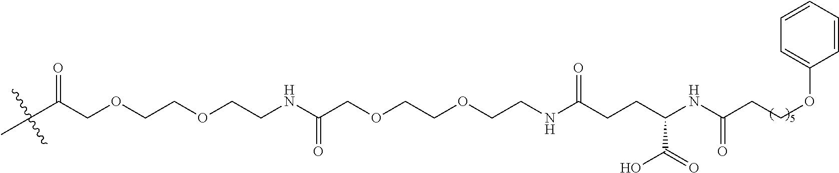

C6XgEPEG2PEG2CO, PEG2PEG2gEC6X

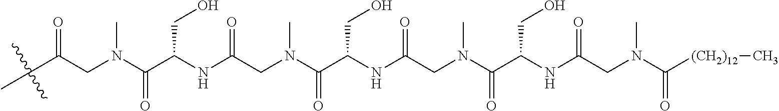

Sar-S-Sar-S-Sar-S-Sar-C14

Further examples of lipophilic substituents are described in WO2023/288019.

In some embodiments, the peptide comprises a sequence according to any one of the following Formulas:

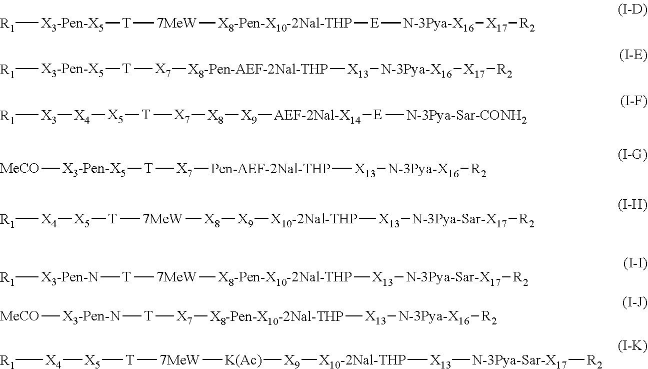

In some embodiments, the amino acid at X₃ is an L-amino acid. In some embodiments, the amino acid at X₃ is a D-amino acid.

In some embodiments, the amino acid at X₄ is an L-amino acid. In some embodiments, the amino acid at X₄ is a D-amino acid.

In some embodiments, the amino acid at X₅ is an L-amino acid. In some embodiments, the amino acid at X₅ is a D-amino acid.

In some embodiments, the amino acid at X₆ is an L-amino acid. In some embodiments, the amino acid at X₆ is a D-amino acid.

In some embodiments, the amino acid at X₇ is an L-amino acid. In some embodiments, the amino acid at X₇ is a D-amino acid.

In some embodiments, the amino acid at X₈ is an L-amino acid. In some embodiments, the amino acid at X₈ is a D-amino acid.

In some embodiments, the amino acid at X₉ is an L-amino acid. In some embodiments, the amino acid at X₉ is a D-amino acid.

In some embodiments, the amino acid at X₁₀ is an L-amino acid. In some embodiments, the amino acid at X₁₀ is a D-amino acid.

In some embodiments, the amino acid at X₁₁ is an L-amino acid. In some embodiments, the amino acid at X₁₁ is a D-amino acid.

In some embodiments, the amino acid at X₁₂ is an L-amino acid. In some embodiments, the amino acid at X₁₂ is a D-amino acid.

In some embodiments, the amino acid at X₁₃ is an L-amino acid. In some embodiments, the amino acid at X₁₃ is a D-amino acid.

In some embodiments, the amino acid at X₁₄ is an L-amino acid. In some embodiments, the amino acid at X₁₄ is a D-amino acid.

In some embodiments, the amino acid at X₁₅ is an L-amino acid. In some embodiments, the amino acid at X₁₅ is a D-amino acid.

In some embodiments, the amino acid at X₁₆ is an L-amino acid. In some embodiments, the amino acid at X₁₆ is a D-amino acid.

In some embodiments, the amino acid at X₁₇ is an L-amino acid. In some embodiments, the amino acid at X₁₇ is a D-amino acid.

In some embodiments, the present disclosure provides a peptide described herein provided the peptide retains activity as an inhibitor of interleukin-23 receptor.

The present disclosure further provides a peptide of any one of SEQ ID NOS: 1-170, as shown in Table 4, or a pharmaceutically acceptable salt thereof.

TABLE 4
Example Peptides

| SEQ | |
|---|---|
| ID | |
| NO | Structure |
| 1 | 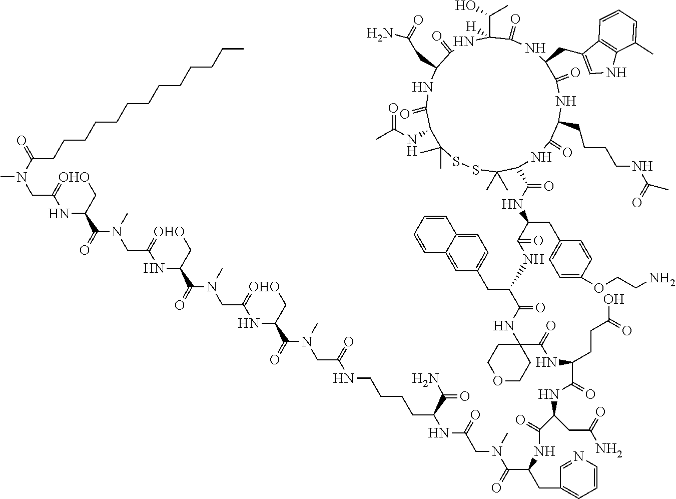 |
| | MeCO-Pen(3)-N-T-7MeW-K(Ac)-Pen(3)-AEF-2Nal-THP-E-N-3Pya-Sar-K(Sar-S-Sar-S- |
| | Sar-S-Sar-C14)-CONH2 |
| 2 | 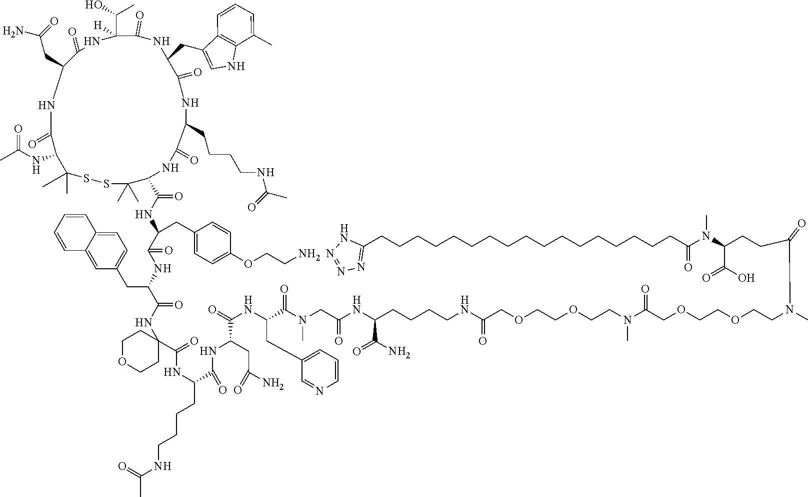 [SMILES: CC(=O)NCCCC[C@H](NC(=O)C1(NC(=O)[C@H](Cc2ccc3ccccc3c2)NC(=O)[C@H](Cc2ccc(OCCN)cc2)NC(=O)[C@@H](NC(=O)[C@@H](N)CCCCNC(C)=O)C(C)(C)SSC(C)(C)[C@H](NC(C)=O)C(=O)N[C@@H](CC(N)=O)C(=O)N[C@H](C(=O)N[C@H](Cc2c[nH]c3c(C)cccc23)C(C)=O)[C@@H](C)O)CCOCC1)C(=O)N[C@@H](CC(N)=O)C(=O)N[C@@H](Cc1cccnc1)C(=O)N(C)CC(=O)N[C@@H](CCCCNC(=O)COCCOCCN(C)C(=O)COCCOCCN(C)C(=O)CC[C@@H](C(=O)O)N(C)C(=O)CCCCCCCCCCCCCCCCCc1nnn[nH]1)C(N)=O] |
| | MeCO-Pen(3)-N-T-7MeW-K(Ac)-Pen(3)-AEF-2Nal-THP-K(Ac)-N-3Pya-Sar- |
| | K(PEG2NMePEG2NMegENMeC18Tetrazole)-CONH2 |
| 3 | 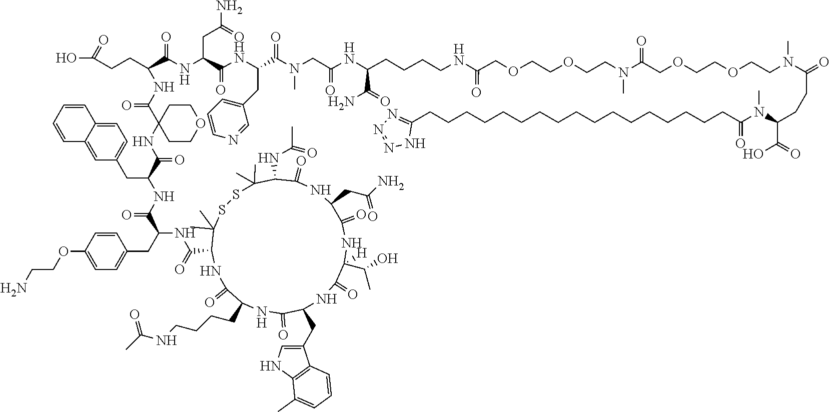 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCOCCN(C)C(=O)COCCOCCN(C)C(=O)CC[C@@H](C(=O)O)N(C)C(=O)CCCCCCCCCCCCCCCCCc3nnn[nH]3)C(N)=O)CCOCC2)NC1=O] |
| | MeCO-Pen(3)-N-T-7MeW-K(Ac)-Pen(3)-AEF-2Nal-THP-E-N-3Pya-Sar- |
| | K(PEG2NMePEG2NMegENMeC18Tetrazole)-CONH2 |
| 4 | 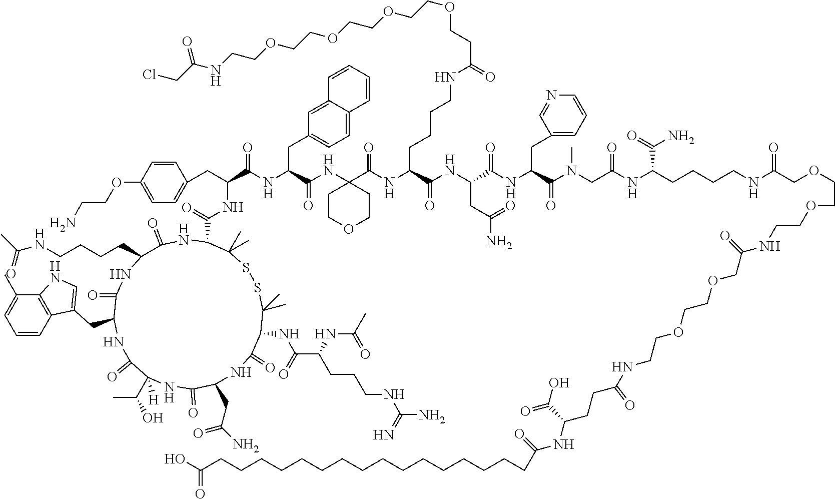 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(=O)CCOCCOCCOCCOCCNC(=O)CCl)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)NC1=O] |
| | MeCO-r-Pen(3)-N-T-7MeW-K(Ac)-Pen(3)-AEF-2Nal-THP-K(PEG4AcCl)-N-3Pya-Sar- |
| | K(PEG2PEG2gEC18OH)-CONH2 |
| 5 | 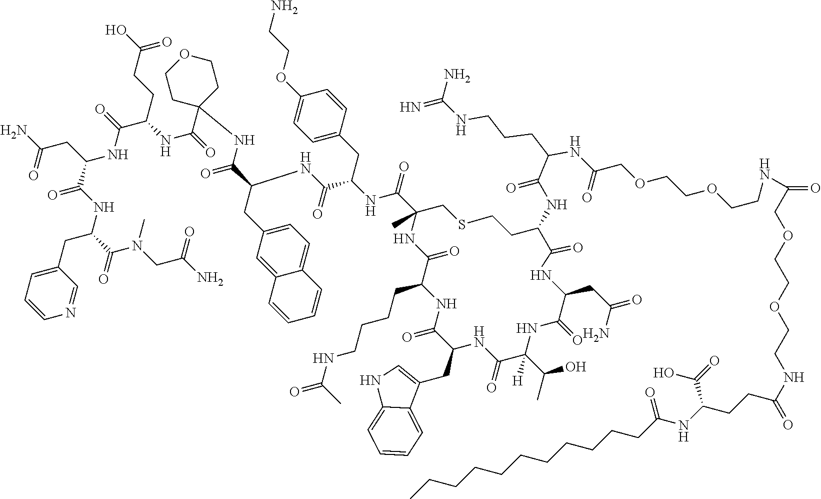 [SMILES: CCCCCCCCCCCC(=O)N[C@@H](CCC(=O)NCCOCCOCC(=O)NCCOCCOCC(=O)NC(CCCNC(=N)N)C(=O)N[C@H]1CCSC[C@@](C)(C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC(=O)[C@H](CCCCNC(C)=O)NC(=O)[C@H](Cc2c[nH]c3ccccc23)NC(=O)[C@H]([C@H](C)O)NC(=O)[C@H](CC(N)=O)NC1=O)C(=O)O] |
| | C12gEPEG2PEG2CO-r-Abu(1)-N-T-W-K(Ac)-aMeC(1)-AEF-2Nal-THP-E-N-3Pya-Sar- |
| | CONH2 |
| 6 | 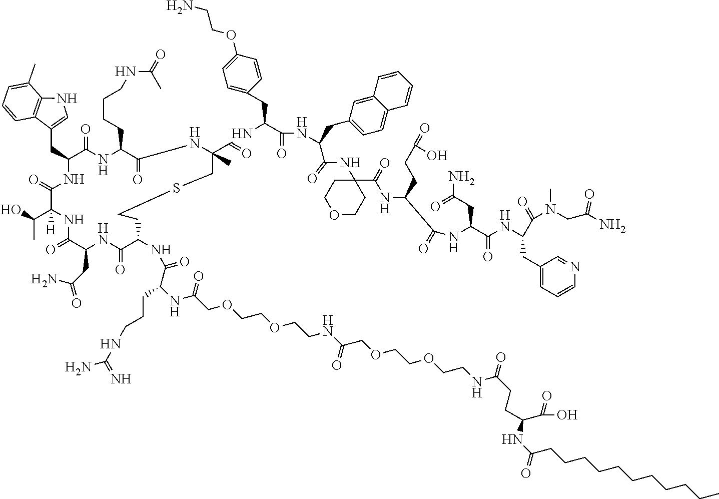 [SMILES: CCCCCCCCCCCC(=O)N[C@@H](CCC(=O)NCCOCCOCC(=O)NCCOCCOCC(=O)N[C@H](CCCNC(=N)N)C(=O)N[C@H]1CCSC[C@@](C)(C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC(=O)[C@H](CCCCNC(C)=O)NC(=O)[C@H](Cc2c[nH]c3c(C)cccc23)NC(=O)[C@H]([C@@H](C)O)NC(=O)[C@H](CC(N)=O)NC1=O)C(=O)O] |
| | C12gEPEG2PEG2CO-r-Abu(1)-N-T-7MeW-K(Ac)-aMeC(1)-AEF-2Nal-THP-E-N-ePya-Sar |
| | CONH2 |
| 7 | 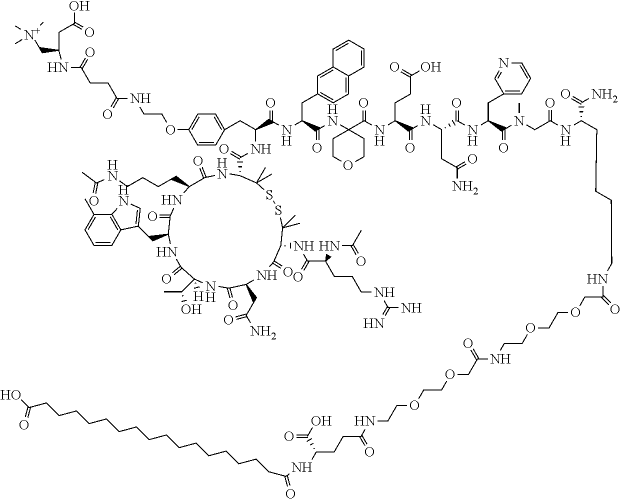 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@H](CCCNC(=N)N)NC(C)=O)C(C)(C)SSC(C)(C)[C@@H](C(=O)N[C@@H](Cc2ccc(OCCNC(=O)CCC(=O)N[C@H](CC(=O)O)C[N+](C)(C)C)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)NC1=O] |
| | C6XgEPEG2PEG2CO-Pen(3)-N(N(Me)2)-T-7MeW-K(Ac)-Pen(3)-TMAPF-2Nal-THP- |
| | K(NMeAc)-N-3Pya-NMeK(PEG2PEG2gEC6X)-CONH2 |
| 8 | 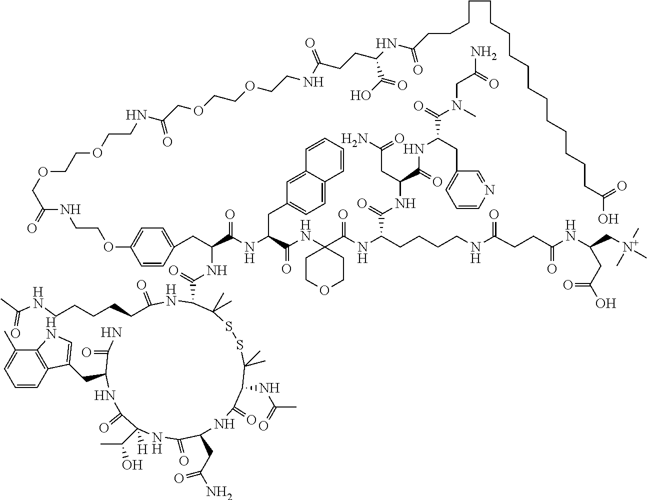 |
| | MeCO-Pen(3)-N-T-7MeW-K(Ac)-Pen(3)-AEF(PEG2PEG2gEC18OH)-2Nal-THP-K(d)-N- |
| | 3Pya-Sar-CONH2 |
| 9 | 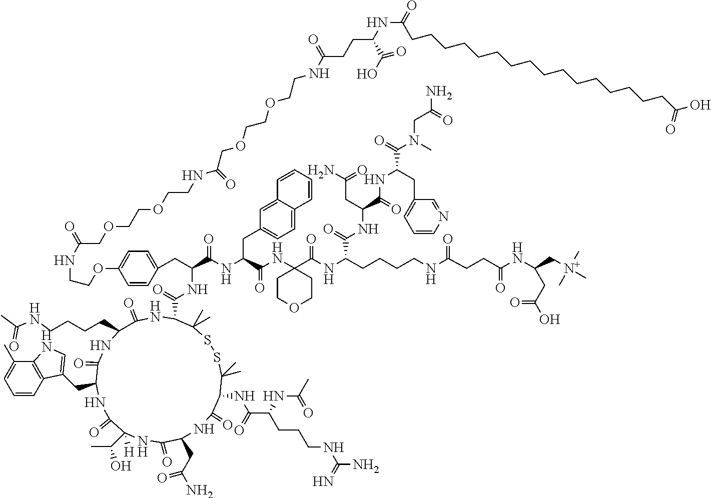 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCNC(=O)COCCOCCNC(=O)COCCOCCNC(=O)CC[C@H](NC(=O)CCCCCCCCCCCCCCCCC(=O)O)C(=O)O)cc2)C(=O)N[C@@H](Cc2ccc3ccccc3c2)C(=O)NC2(C(=O)N[C@@H](CCCCNC(=O)CCC(=O)N[C@H](CC(=O)O)C[N+](C)(C)C)C(=O)N[C@@H](CC(N)=O)C(=O)N[C@@H](Cc3cccnc3)C(=O)N(C)CC(N)=O)CCOCC2)NC1=O] |
| | MeCO-r-Pen(3)-N-T-7MeW-K(Ac)-Pen(3)-AEF(PEG2PEG2gEC18OH)-2Nal-THP-K(d)-N- |
| | 3Pya-Sar-CONH2 |
| 10 | 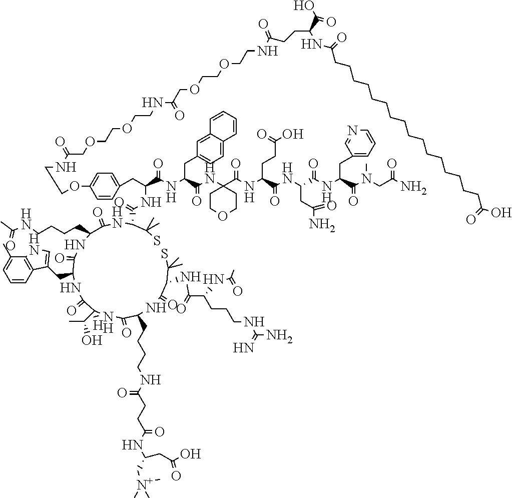 [SMILES: CC(=O)NCCCC[C@@H]1NC(=O)[C@H](Cc2c[nH]c3c(C)cccc23)NC(=O)[C@H]([C@@H](C)O)NC(=O)[C@H](CCCCNC(=O)CCC(=O)N[C@H](CC(=O)O)C[N+](C)(C)C)NC(=O)[C@@H](NC(=O)[C@@H](CCCNC(=N)N)NC(C)=O)C(C)(C)SSC(C)(C)[C@@H](C(=O)N[C@@H](Cc2ccc(OCCNC(=O)COCCOCCNC(=O)COCCOCCNC(=O)CC[C@H](NC(=O)CCCCCCCCCCCCCCCCC(=O)O)C(=O)O)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O] |
| | MeCO-r-Pen(3)-K(d)-T-7MeW-K(Ac)-Pen(3)-AEF(PEG2PEG2gEC18OH)-2Nal-THP-E-N- |
| | 3Pya-Sar-CONH2 |
| 11 | 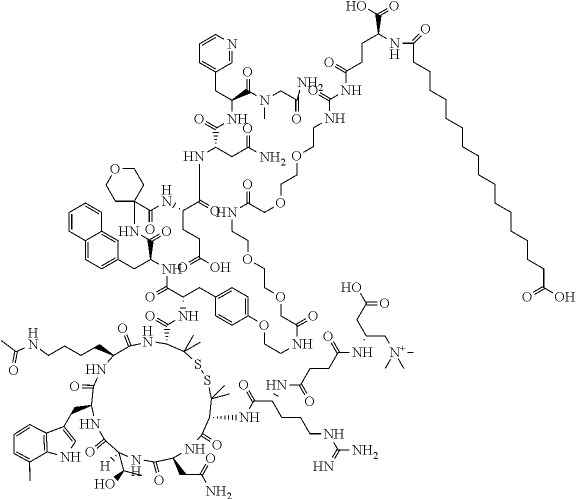 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(=O)CCC(=O)N[C@H](CC(=O)O)C[N+](C)(C)C)C(C)(C)SSC(C)(C)[C@@H](C(=O)N[C@@H](Cc2ccc(OCCNC(=O)COCCOCCNC(=O)COCCOCCNC(=O)NC(=O)CC[C@H](NC(=O)CCCCCCCCCCCCCCCCC(=O)O)C(=O)O)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O] |
| | succiniccarn-r-Pen(3)-N-T-7MeW-K(Ac)-Pen(3)-AEF(PEG2PEG2gEC18OH)-2Nal-THP-E- |
| | N-3Pya-Sar-CONH2 |
| 12 | 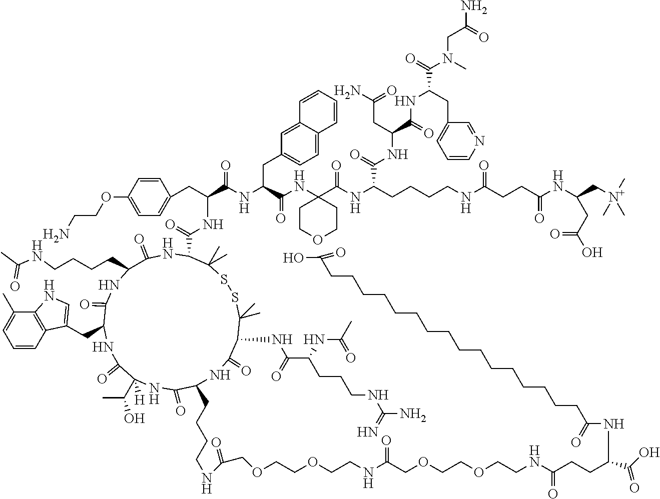 [SMILES: CC(=O)NCCCC[C@@H]1NC(=O)[C@H](Cc2c[nH]c3c(C)cccc23)NC(=O)[C@H]([C@@H](C)O)NC(=O)[C@H](CCCCNC(=O)COCCOCCNC(=O)COCCOCCNC(=O)CC[C@H](NC(=O)CCCCCCCCCCCCCCCCC(=O)O)C(=O)O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(=O)CCC(=O)N[C@H](CC(=O)O)C[N+](C)(C)C)C(=O)N[C@@H](CC(N)=O)C(=O)N[C@@H](Cc3cccnc3)C(=O)N(C)CC(N)=O)CCOCC2)NC1=O] |
| | MeCO-r-Pen(3)-K(PEG2PEG2gEC18OH)-T-7MeW-K(Ac)-Pen(3)-AEF-2Nal-THP-K(d)-N- |
| | 3Pya-Sar-CONH2 |
| 13 | 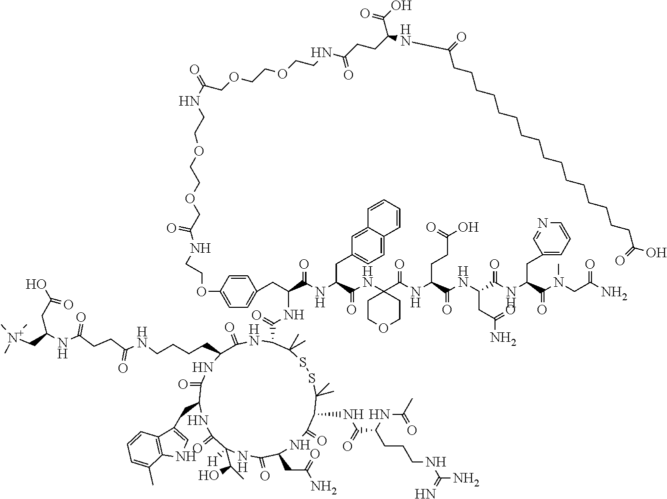 [SMILES: CC(=O)N[C@H](CCCNC(=N)N)C(=O)N[C@@H]1C(=O)N[C@@H](CC(N)=O)C(=O)N[C@@H]([C@@H](C)O)C(=O)N[C@@H](Cc2c[nH]c3c(C)cccc23)C(=O)N[C@@H](CCCCNC(=O)CCC(=O)N[C@H](CC(=O)O)C[N+](C)(C)C)C(=O)N[C@H](C(=O)N[C@@H](Cc2ccc(OCCNC(=O)COCCOCCNC(=O)COCCOCCNC(=O)CC[C@H](NC(=O)CCCCCCCCCCCCCCCCC(=O)O)C(=O)O)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)C(C)(C)SSC1(C)C] |
| | MeCO-r-Pen(3)-N-T-7MeW-K(d)-Pen(3)-AEF(PEG2gEC18OH)-2Nal-THP-E-N-3Pya- |
| | Sar-CONH2 |
| 14 | 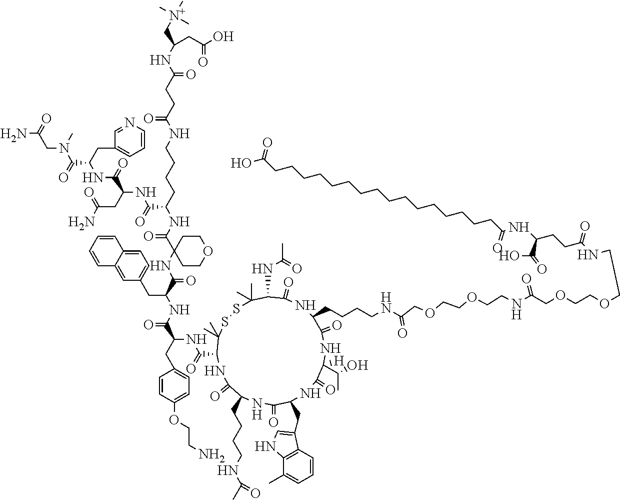 [SMILES: CC(=O)NCCCC[C@@H]1NC(=O)[C@H](Cc2c[nH]c3c(C)cccc23)NC(=O)[C@H]([C@@H](C)O)NC(=O)[C@H](CCCCNC(=O)COCCOCCNC(=O)COCCOCCNC(=O)CC[C@H](NC(=O)CCCCCCCCCCCCCCCCC(=O)O)C(=O)O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(=O)CCC(=O)N[C@H](CC(=O)O)C[N+](C)(C)C)C(=O)N[C@@H](CC(N)=O)C(=O)N[C@@H](Cc3cccnc3)C(=O)N(C)CC(N)=O)CCOCC2)NC1=O] |
| | MeCO-Pen(3)-K(PEG2PEG2gEC18OH)-T-7MeW-K(Ac)-Pen(3)-AEF-2Nal-THP-K(d)-N- |
| | 3Pya-Sar-CONH2 |
| 15 | 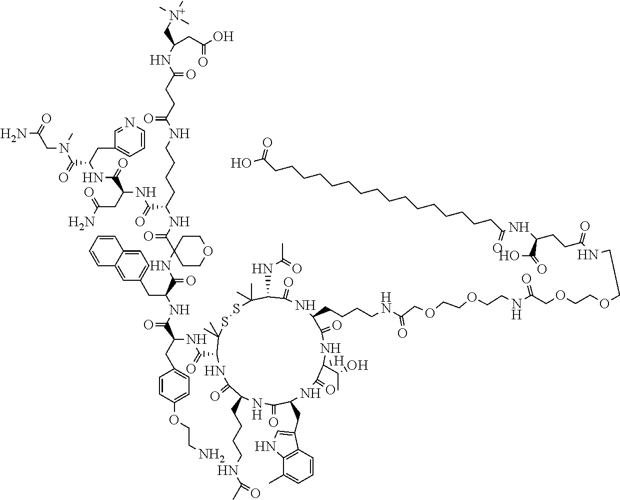 [SMILES: CC(=O)NCCCC[C@@H]1NC(=O)[C@H](Cc2c[nH]c3c(C)cccc23)NC(=O)[C@H]([C@@H](C)O)NC(=O)[C@H](CCCCNC(=O)COCCOCCNC(=O)COCCOCCNC(=O)CC[C@H](NC(=O)CCCCCCCCCCCCCCCCC(=O)O)C(=O)O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(=O)CCC(=O)N[C@H](CC(=O)O)C[N+](C)(C)C)C(=O)N[C@@H](CC(N)=O)C(=O)N[C@@H](Cc3cccnc3)C(=O)N(C)CC(N)=O)CCOCC2)NC1=O] |
| | MeCO-r-Pen(3)-N-T-7MeW-K(PEG2PEG2gEC18OH)-Pen(3)-AEF-2Nal-THP-K(d)-N-3Pya- |
| | Sar-CONH2 |
| 16 | 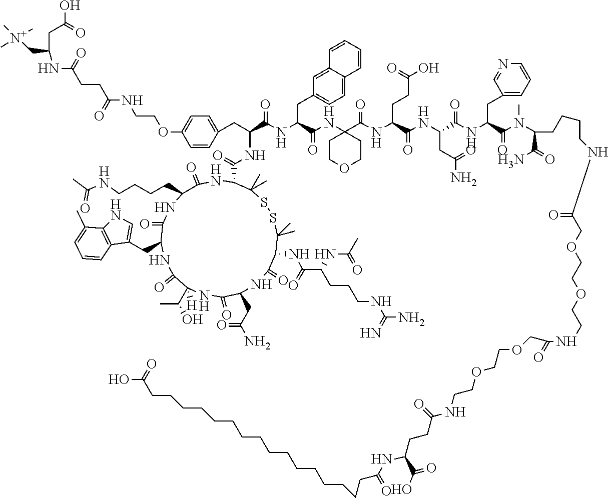 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCNC(=O)CCC(=O)N[C@H](CC(=O)O)C[N+](C)(C)C)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)NC1=O] |
| | MeCO-r-Pen(3)-N-T-7MeW-K(Ac)-Pen(3)-AEF(d)-2Nal-THP-E-N-3Pya- |
| | NMeK(PEG2PEG2gEC18OH)-CONH2 |
| 17 | 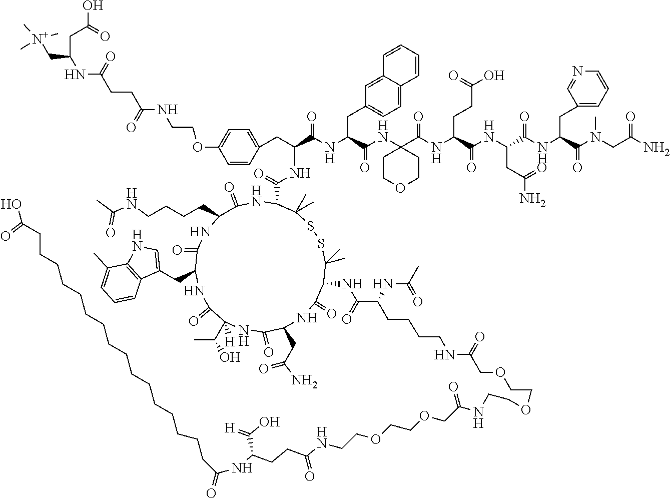 |
| | MeCO-k(PEG2PEG2gEC18OH)-Pen(3)-N-T-7MeW-K(Ac)-Pen(3)-AEF(d)-2Nal-THP-E-N- |
| | 3Pya-Sar-CONH2 |
| 18 | 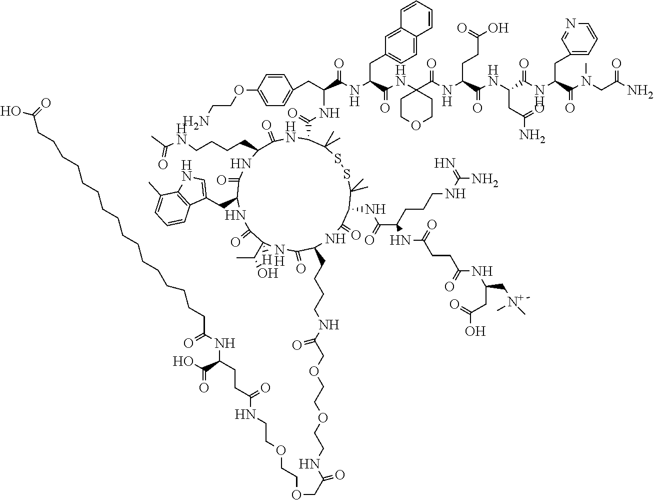 [SMILES: CC(=O)NCCCC[C@@H]1NC(=O)[C@H](Cc2c[nH]c3c(C)cccc23)NC(=O)[C@H]([C@@H](C)O)NC(=O)[C@H](CCCCNC(=O)COCCOCCNC(=O)COCCOCCNC(=O)CC[C@H](NC(=O)CCCCCCCCCCCCCCCCC(=O)O)C(=O)O)NC(=O)[C@@H](NC(=O)[C@@H](CCCNC(=N)N)NC(=O)CCC(=O)N[C@H](CC(=O)O)C[N+](C)(C)C)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O] |
| | succiniccarn-r-Pen(3)-K(PEG2PEG2gEC18OH)-T-7MeW-K(Ac)-Pen(3)-AEF-2Nal-THP-E- |
| | N-3Pya-Sar-CONH2 |
| 19 | 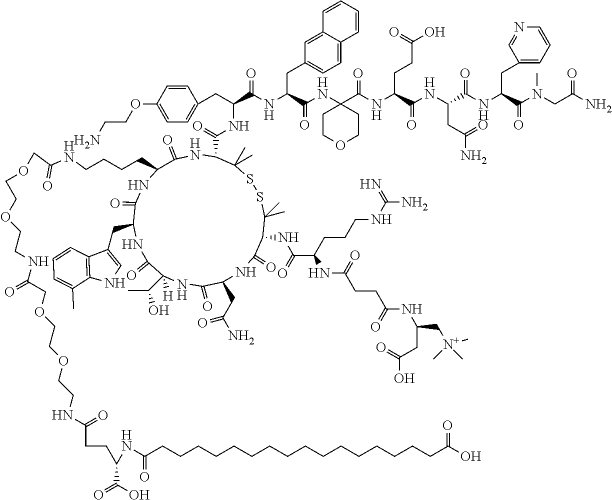 [SMILES: Cc1cccc2c(C[C@@H]3NC(=O)[C@H]([C@@H](C)O)NC(=O)[C@H](CC(N)=O)NC(=O)[C@@H](NC(=O)[C@@H](CCCNC(=N)N)NC(=O)CCC(=O)N[C@H](CC(=O)O)C[N+](C)(C)C)C(C)(C)SSC(C)(C)[C@@H](C(=O)N[C@@H](Cc4ccc(OCCN)cc4)C(=O)N[C@@H](Cc4ccc5ccccc5c4)C(=O)NC4(C(=O)N[C@@H](CCC(=O)O)C(=O)N[C@@H](CC(N)=O)C(=O)N[C@@H](Cc5cccnc5)C(=O)N(C)CC(N)=O)CCOCC4)NC(=O)[C@H](CCCCNC(=O)COCCOCCNC(=O)COCCOCCNC(=O)CC[C@H](NC(=O)CCCCCCCCCCCCCCCCC(=O)O)C(=O)O)NC3=O)c[nH]c12] |
| | MeCO-r-Pen(3)-N-T-7MeW-K(PEG2PEG2gEC18OH)-Pen(3)-AEF-2Nal-THP-E-N-3Pya- |
| | NMeK(d)-CONH2 |
| 20 | 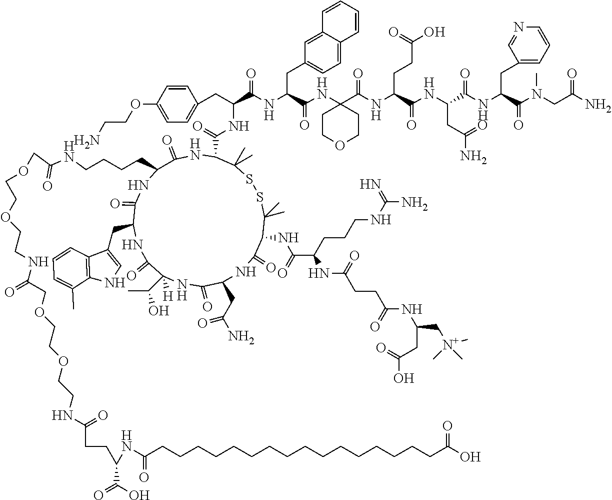 [SMILES: Cc1cccc2c(C[C@@H]3NC(=O)[C@H]([C@@H](C)O)NC(=O)[C@H](CC(N)=O)NC(=O)[C@@H](NC(=O)[C@@H](CCCNC(=N)N)NC(=O)CCC(=O)N[C@H](CC(=O)O)C[N+](C)(C)C)C(C)(C)SSC(C)(C)[C@@H](C(=O)N[C@@H](Cc4ccc(OCCN)cc4)C(=O)N[C@@H](Cc4ccc5ccccc5c4)C(=O)NC4(C(=O)N[C@@H](CCC(=O)O)C(=O)N[C@@H](CC(N)=O)C(=O)N[C@@H](Cc5cccnc5)C(=O)N(C)CC(N)=O)CCOCC4)NC(=O)[C@H](CCCCNC(=O)COCCOCCNC(=O)COCCOCCNC(=O)CC[C@H](NC(=O)CCCCCCCCCCCCCCCCC(=O)O)C(=O)O)NC3=O)c[nH]c12] |
| | succiniccarn-r-Pen(3)-N-T-7MeW-K(PEG2PEG2gEC18OH)-Pen(3)-AEF-2Nal-THP-E-N- |
| | 3Pya-Sar-CONH2 |
| 21 | 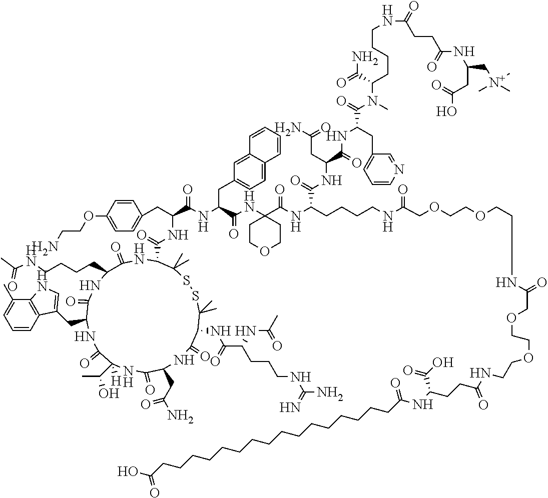 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(=O)N[C@@H](CC(N)=O)C(=O)N[C@@H](Cc3cccnc3)C(=O)N(C)[C@@H](CCCCNC(=O)CCC(=O)N[C@H](CC(=O)O)C[N+](C)(C)C)C(N)=O)CCOCC2)NC1=O] |
| | MeCO-r-Pen(3)-N-T-7MeW-K(Ac)-Pen(3)-AEF-2Nal-THP-K(PEG2PEG2gEC18OH)-N- |
| | 3Pya-NMeK(d)-CONH2 |
| 22 | 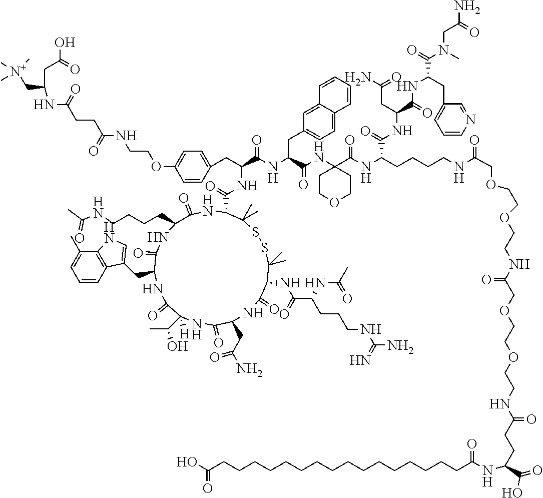 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCNC(=O)CCC(=O)N[C@H](CC(=O)O)C[N+](C)(C)C)cc2)C(=O)N[C@@H](Cc2ccc3ccccc3c2)C(=O)NC2(C(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(=O)N[C@@H](CC(N)=O)C(=O)N[C@@H](Cc3cccnc3)C(=O)N(C)CC(N)=O)CCOCC2)NC1=O] |
| | MeCO-r-Pen(3)-N-T-7MeW-K(Ac)-Pen(3)-AEF(d)-2Nal-THP-K(PEG2PEG2gEC18OH)-N- |
| | 3Pya-Sar-CONH2 |
| 23 | 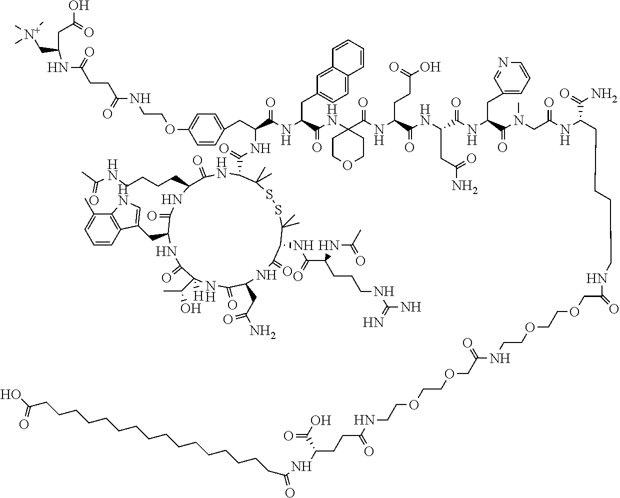 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@H](CCCNC(=N)N)NC(C)=O)C(C)(C)SSC(C)(C)[C@@H](C(=O)N[C@@H](Cc2ccc(OCCNC(=O)CCC(=O)N[C@H](CC(=O)O)C[N+](C)(C)C)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)NC1=O] |
| | MeCO-r-Pen(3)-N-T-7MeW-K(Ac)-Pen(3)-AEF(d)-2Nal-THP-E-N-3Pya-Sar- |
| | K(PEG2PEG2gEC18OH)-CONH2 |
| 24 | 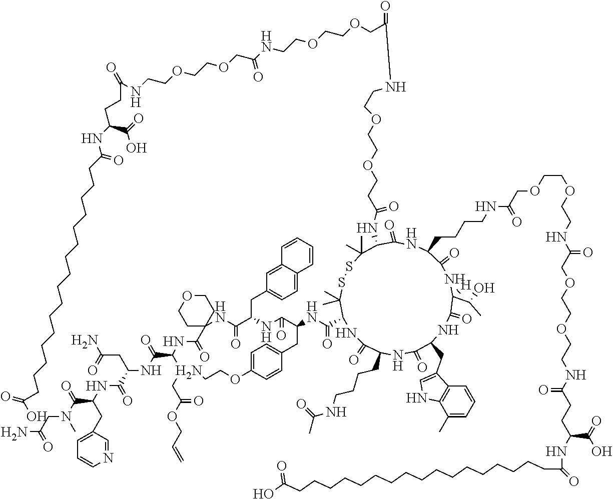 [SMILES: C=CCOC(=O)CN1CCOc2ccc(cc2)C[C@H](NC(=O)[C@H]2NC(=O)[C@H](CCCCNC(C)=O)NC(=O)[C@H](Cc3c[nH]c4c(C)cccc34)NC(=O)[C@H]([C@@H](C)O)NC(=O)[C@H](CCCCNC(=O)COCCOCCNC(=O)COCCOCCNC(=O)CC[C@H](NC(=O)CCCCCCCCCCCCCCCCCC(=O)O)C(=O)O)NC(=O)[C@@H](NC(=O)CCOCCOCCNC(=O)COCCOCCNC(=O)COCCOCCNC(=O)CC[C@H](NC(=O)CCCCCCCCCCCCCCCCC(=O)O)C(=O)O)C(C)(C)SSC2(C)C)C(=O)N[C@@H](Cc2ccc3ccccc3c2)C(=O)NC2(CCOCC2)C(=O)N[C@@H]1C(=O)N[C@@H](CC(N)=O)C(=O)N[C@@H](Cc1cccnc1)C(=O)N(C)CC(N)=O] |
| | AEEP(PEG2PEG2gEC18OH)-Pen(3)-K(PEG2PEG2gEC18OH)-T-7MeW-K(Ac)-Pen(3)- |
| | AEF-2Nal-THP-E(OAll)-N-3Pya-Sar-CONH2 |
| 25 | 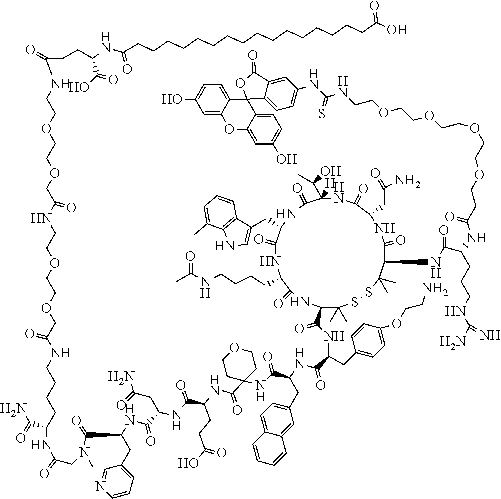 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(=O)CCOCCOCCOCCOCCNC(=S)Nc2ccc3c(c2)C(=O)OC32c3ccc(O)cc3Oc3cc(O)ccc32)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)NC1=O] |
| | FITCPEG4CO-r-Pen(3)-N-T-7MeW-K(Ac)-Pen(3)-AEF-2Nal-THP-E-N-3Pya-Sar- |
| | K(PEG2PEG2gEC18OH)-CONH2 |
| 26 | 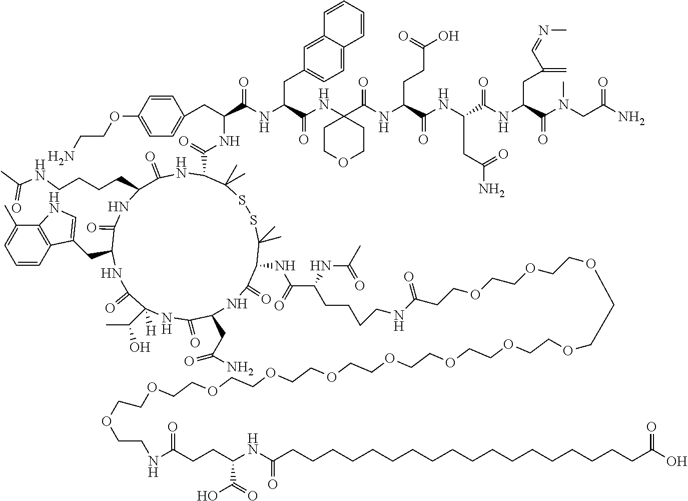 [SMILES: C=C(/C=N\C)C[C@H](NC(=O)[C@H](CC(N)=O)NC(=O)[C@H](CCC(=O)O)NC(=O)C1(NC(=O)[C@H](Cc2ccc3ccccc3c2)NC(=O)[C@H](Cc2ccc(OCCN)cc2)NC(=O)[C@H]2NC(=O)[C@H](CCCCNC(C)=O)NC(=O)[C@H](Cc3c[nH]c4c(C)cccc34)NC(=O)[C@H]([C@@H](C)O)NC(=O)[C@H](CC(N)=O)NC(=O)[C@@H](NC(=O)[C@@H](CCCCNC(=O)CCOCCOCCOCCOCCOCCOCCOCCOCCOCCOCCOCCOCCNC(=O)CC[C@H](NC(=O)CCCCCCCCCCCCCCCCCCC(=O)O)C(=O)O)NC(C)=O)C(C)(C)SSC2(C)C)CCOCC1)C(=O)N(C)CC(N)=O] |
| | MeCO-k(PEG12gEC20OH)-Pen(3)-N-T-7MeW-K(Ac)-Pen(3)-AEF-2Nal-THP-E-N-3Pya- |
| | Sar-CONH2 |
| 27 | 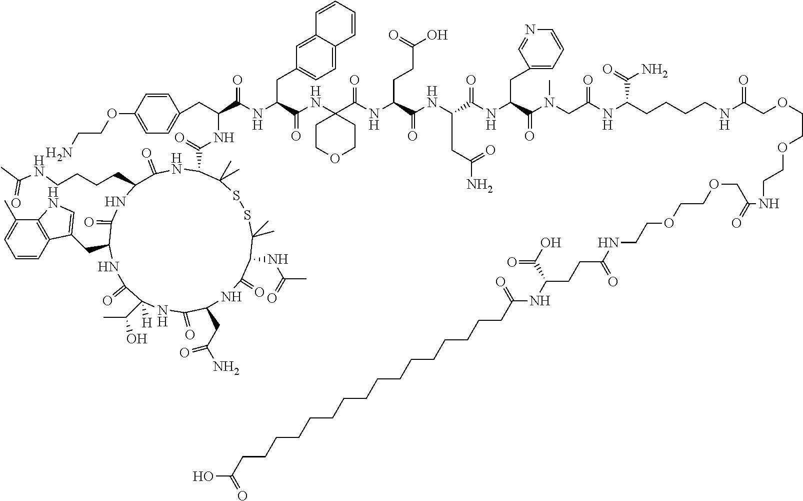 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)NC1=O] |
| | MeCO-Pen(3)-N-T-7MeW-K(Ac)-Pen(3)-AEF-2Nal-THP-E-N-3Pya-Sar- |
| | K(PEG2PEG2gEC18OH)-CONH2 |
| 28 | 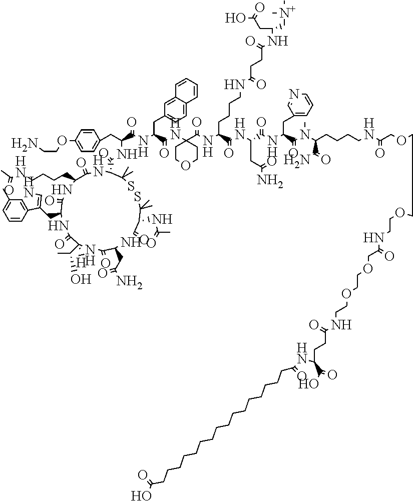 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(=O)CCC(=O)N[C@H](CC(=O)O)C[N+](C)(C)C)C(=O)N[C@@H](CC(N)=O)C(=O)N[C@@H](Cc3cccnc3)C(=O)N(C)[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)NC1=O] |
| | MeCO-Pen(3)-N-T-7MeW-K(Ac)-Pen(3)-AEF-2Nal-THP-K(d)-N-3Pya- |
| | NMeK(PEG2PEG2gEC18OH)-CONH2 |
| 29 | 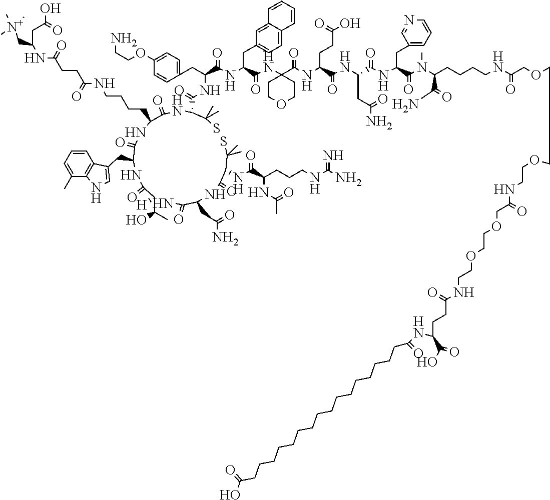 [SMILES: CC(=O)N[C@H](CCCNC(=N)N)C(=O)N[C@@H]1C(=O)N[C@@H](CC(N)=O)C(=O)N[C@@H]([C@@H](C)O)C(=O)N[C@@H](Cc2c[nH]c3c(C)cccc23)C(=O)N[C@@H](CCCCNC(=O)CCC(=O)N[C@H](CC(=O)O)C[N+](C)(C)C)C(=O)N[C@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)C(C)(C)SSC1(C)C] |
| | MeCO-r-Pen(3)-N-T-7MeW-K(D)-Pen(3)-AEF-2Nal-THP-E-N-3Pya- |
| | NMeK(PEG2PEG2gEC18OH)-CONH2 |
| 30 | 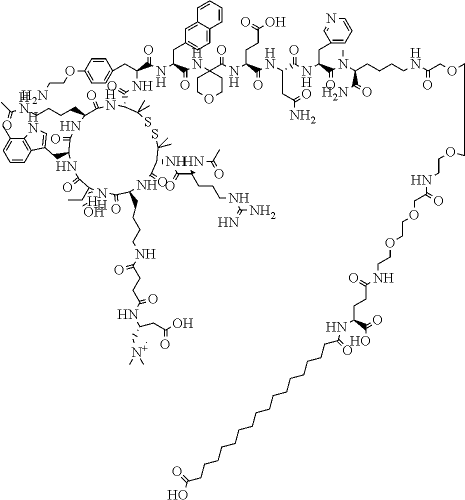 [SMILES: CC(=O)NCCCC[C@@H]1NC(=O)[C@H](Cc2c[nH]c3c(C)cccc23)NC(=O)[C@H]([C@@H](C)O)NC(=O)[C@H](CCCCNC(=O)CCC(=O)N[C@H](CC(=O)O)C[N+](C)(C)C)NC(=O)[C@@H](NC(=O)[C@H](CCCNC(=N)N)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)NC1=O] |
| | MeCO-r-Pen(3)-K(d)-T-7MeW-K(Ac)-Pen(3)-AEF-2Nal-THP-E-N-3Pya- |
| | NMeK(PEG2PEG2gEC18OH)-CONH2 |
| 31 | 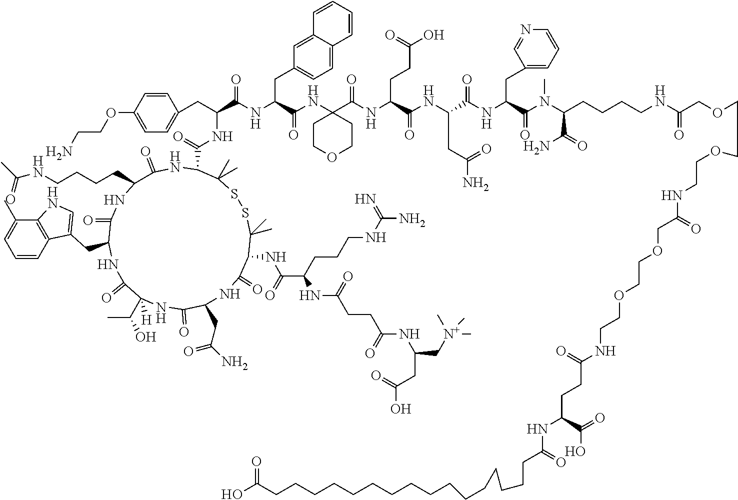 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(=O)CCC(=O)N[C@H](CC(=O)O)C[N+](C)(C)C)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)NC1=O] |
| | succiniccarn-r-Pen(3)-N-T-7MeW-K(Ac)-Pen(3)-AEF-2Nal-THP-E-N-3Pya- |
| | NMeK(PEG2PEG2gEC18OH)-CONH2 |
| 32 | 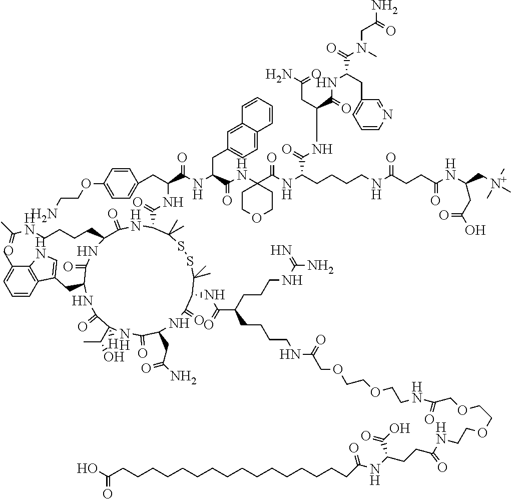 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@H](CCCCNC(=O)COCCOCCNC(=O)COCCOCCNC(=O)CC[C@H](NC(=O)CCCCCCCCCCCCCCCCC(=O)O)C(=O)O)CCCNC(=N)N)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(=O)CCC(=O)N[C@H](CC(=O)O)C[N+](C)(C)C)C(=O)N[C@@H](CC(N)=O)C(=O)N[C@@H](Cc3cccnc3)C(=O)N(C)CC(N)=O)CCOCC2)NC1=O] |
| | HOC18gEPEG2PEG2CO-r-Pen(3)-N-T-7MeW-K(Ac)-Pen(3)-AEF(d)-2Nal-THP-E-N-3Pya- |
| | Sar-CONH2 |
| 33 | 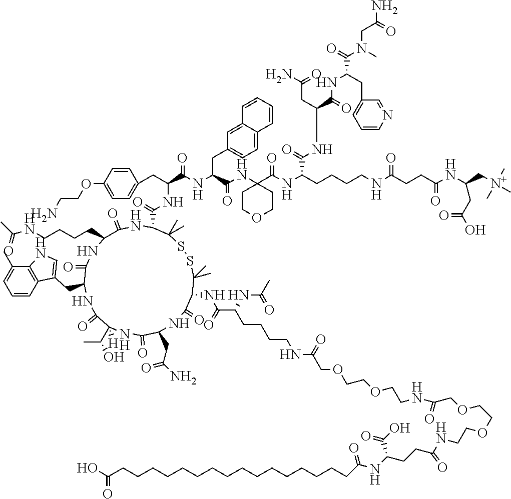 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCCNC(=O)COCCOCCNC(=O)COCCOCCNC(=O)CC[C@H](NC(=O)CCCCCCCCCCCCCCCCC(=O)O)C(=O)O)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(=O)CCC(=O)N[C@H](CC(=O)O)C[N+](C)(C)C)C(=O)N[C@@H](CC(N)=O)C(=O)N[C@@H](Cc3cccnc3)C(=O)N(C)CC(N)=O)CCOCC2)NC1=O] |
| | MeCO-k(PEG2PEG2gEC18OH)-Pen(3)-N-T-7MeW-K(Ac)-Pen(3)-AEF-2Nal-THP-K(d)-N- |
| | 3Pya-Sar-CONH2 |
| 34 | 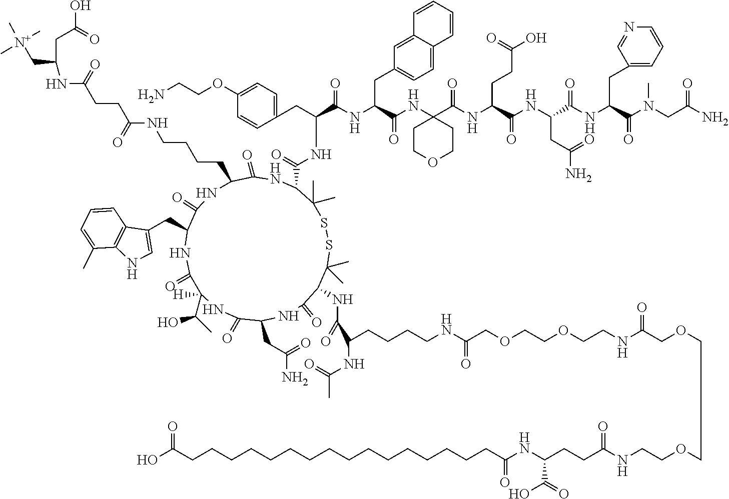 |
| | MeCO-k(PEG2PEG2gEC18OH)-Pen(3)-N-T-7MeW-K(d)-Pen(3)-AEF-2Nal-THP-E-N- |
| | 3Pya-Sar-CONH2 |
| 35 | 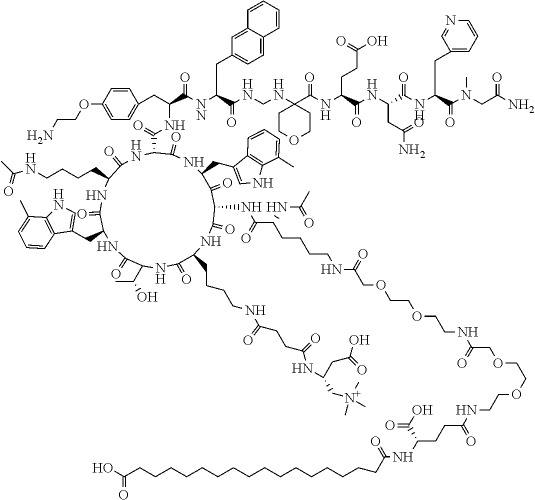 |
| | MeCO-k(PEG2PEG2gEC18OH)-Pen(3)-K(d)-T-7MeW-K(Ac)-Pen(3)-AEF-2Nal-THP-E-N- |
| | 3Pya-Sar-CONH2 |
| 36 | 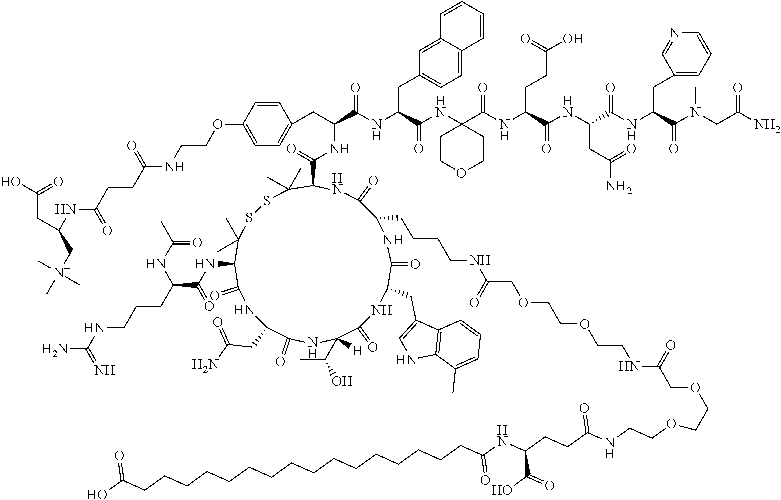 [SMILES: CC(=O)N[C@H](CCCNC(=N)N)C(=O)N[C@@H]1C(=O)N[C@@H](CC(N)=O)C(=O)N[C@@H]([C@@H](C)O)C(=O)N[C@@H](Cc2c[nH]c3c(C)cccc23)C(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(=O)N[C@H](C(=O)N[C@@H](Cc2ccc(OCCNC(=O)CCC(=O)N[C@H](CC(=O)O)C[N+](C)(C)C)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)C(C)(C)SSC1(C)C] |
| | MeCO-r-Pen(3)-N-T-7MeW-K(PEG2PEG2gEC18OH)-Pen(3)-AEF(d)-2Nal-THP-E-N-3Pya- |
| | Sar-CONH2 |
| 37 | 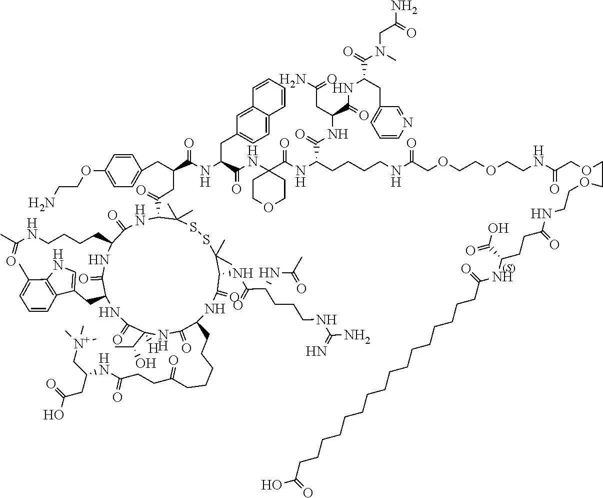 |
| | MeCO-r-Pen(3)-K(d)-T-7MeW-K(Ac)-Pen(3)-AEF-2Nal-THP-K(PEG2PEG2gEC18OH)-N- |
| | 3Pya-Sar-CONH2 |
| 38 | 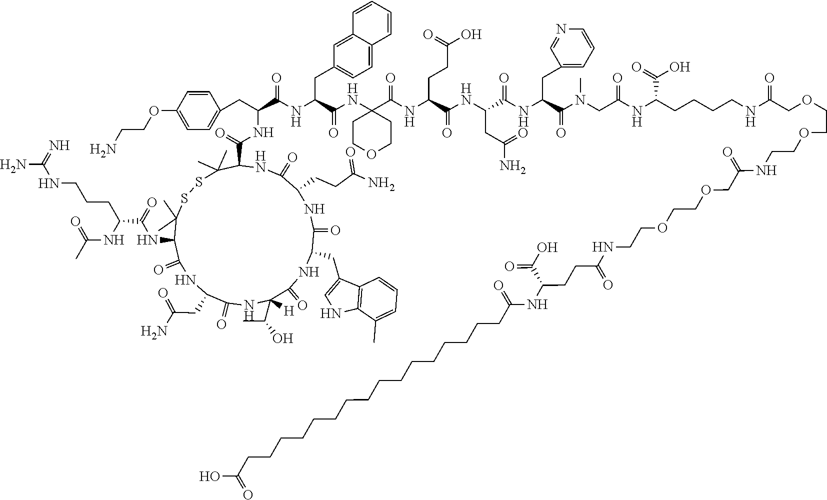 [SMILES: CC(=O)N[C@H](CCCNC(=N)N)C(=O)N[C@@H]1C(=O)N[C@@H](CC(N)=O)C(=O)N[C@@H]([C@@H](C)O)C(=O)N[C@@H](Cc2c[nH]c3c(C)cccc23)C(=O)N[C@@H](CCC(N)=O)C(=O)N[C@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(=O)O)CCOCC2)C(C)(C)SSC1(C)C] |
| | MeCO-r-Pen(3)-N-T-7MeW-Q-Pen(3)-AEF-2Nal-THP-E-N-3Pya-Sar- |
| | K(PEG2PEG2gEC18OH)-CONH2 |
| 39 | 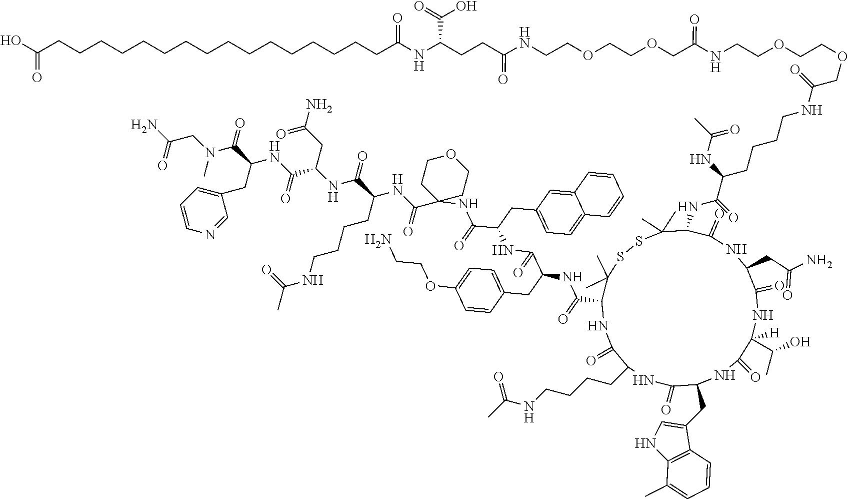 [SMILES: CC(=O)NCCCCC1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@H](CCCCNC(=O)COCCOCCNC(=O)COCCOCCNC(=O)CC[C@H](NC(=O)CCCCCCCCCCCCCCCCC(=O)O)C(=O)O)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC1=O] |
| | MeCO-K(PEG2PEG2gEC18OH)-Pen(3)-N-T-7MeW-K(Ac)-Pen(3)-AEF-2Nal-THP-K(Ac)- |
| | N-3Pya-Sar-CONH2 |
| 40 | 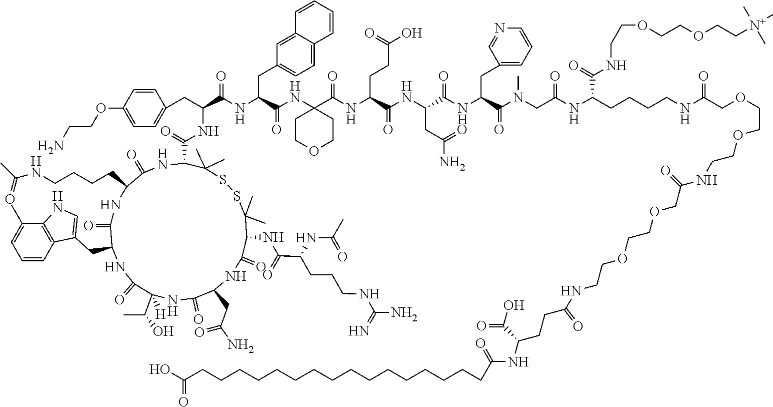 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(=O)NCCOCCOCC[N+](C)(C)C)CCOCC2)NC1=O] |
| | MeCO-r-Pen(3)-N-T-7MeW-K(Ac)-Pen(3)-AEF-2Nal-THP-E-N-3Pya-Sar- |
| | K(PEG2PEG2gEC18OH)-CONH(PEG3a) |
| 41 | 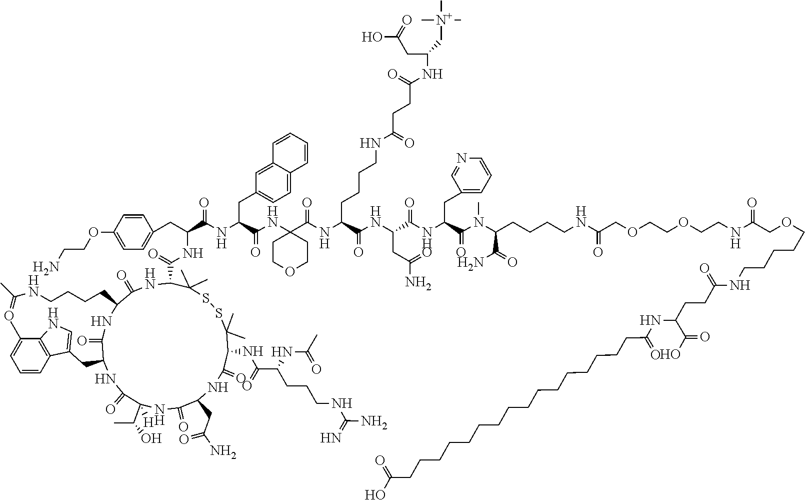 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(=O)CCC(=O)N[C@H](CC(=O)O)C[N+](C)(C)C)C(=O)N[C@@H](CC(N)=O)C(=O)N[C@@H](Cc3cccnc3)C(=O)N(C)[C@@H](CCCCNC(=O)COCCOCCNC(=O)COCCCCCNC(=O)CCC(NC(=O)CCCCCCCCCCCCCCCCC(=O)O)C(=O)O)C(N)=O)CCOCC2)NC1=O] |
| | MeCO-r-Pen(3)-N-T-7MeW-K(Ac)-Pen(3)-AEF-2Nal-THP-K(d)-N-3Pya- |
| | NMeK(PEG2PEG2gEC18OH)-CONH2 |
| 42 | 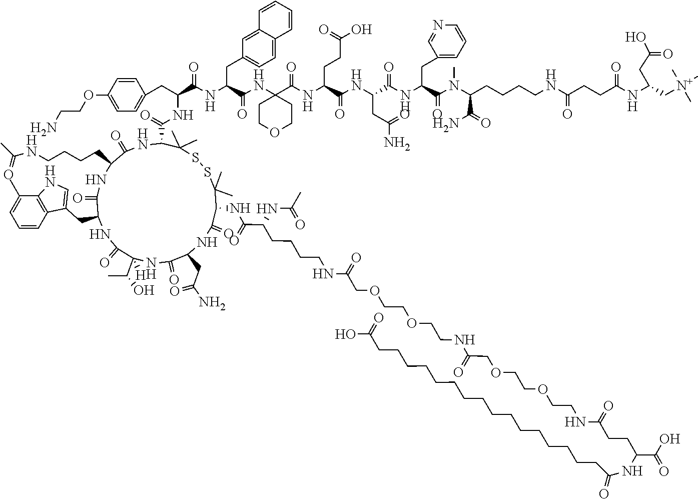 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCCNC(=O)COCCOCCNC(=O)COCCOCCNC(=O)CCC(NC(=O)CCCCCCCCCCCCCCCCC(=O)O)C(=O)O)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)[C@@H](CCCCNC(=O)CCC(=O)N[C@H](CC(=O)O)C[N+](C)(C)C)C(N)=O)CCOCC2)NC1=O] |
| | MeCO-k(PEG2PEG2gEC18OH)-Pen(3)-N-T-7MeW-K(Ac)-Pen(3)-AEF-2Nal-THP-E-N- |
| | 3Pya-NMeK(d)-CONH2 |
| 43 | 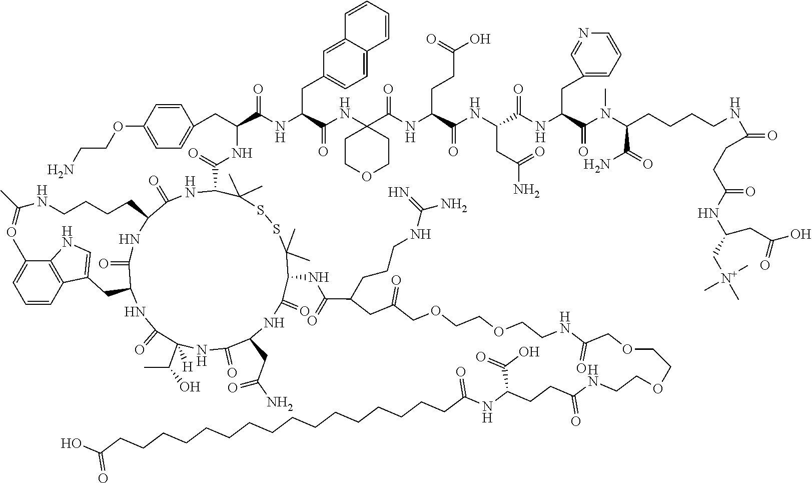 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)C(CCCNC(=N)N)CC(=O)COCCOCCNC(=O)COCCOCCNC(=O)CC[C@H](NC(=O)CCCCCCCCCCCCCCCCC(=O)O)C(=O)O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)[C@@H](CCCCNC(=O)CCC(=O)N[C@H](CC(=O)O)C[N+](C)(C)C)C(N)=O)CCOCC2)NC1=O] |
| | HOC18gEPEG2PEG2CO-r-Pen(3)-N-T-7MeW-K(Ac)-Pen(3)-AEF-2Nal-THP-E-N-3Pya- |
| | NMeK(d)-CONH2 |
| 44 | 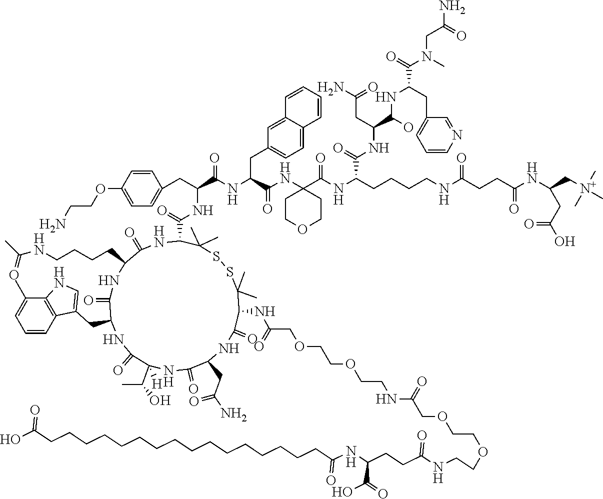 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)COCCOCCNC(=O)COCCOCCNC(=O)CC[C@H](NC(=O)CCCCCCCCCCCCCCCCC(=O)O)C(=O)O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(=O)CCC(=O)N[C@H](CC(=O)O)C[N+](C)(C)C)C(=O)N[C@@H](CC(N)=O)C(O)N[C@@H](Cc3cccnc3)C(=O)N(C)CC(N)=O)CCOCC2)NC1=O] |
| | HOC18gEPEG2PEG2CO-Pen(3)-N-T-7MeW-K(Ac)-Pen(3)-AEF-2Nal-THP-K(d)-N-3Pya- |
| | Sar-CONH2 |
| 45 | 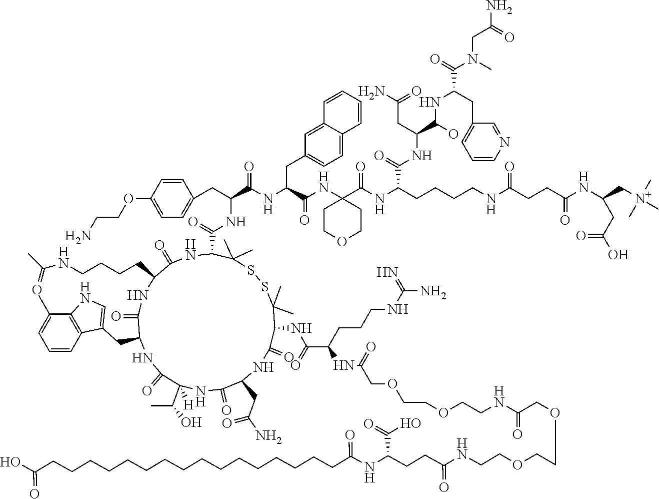 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(=O)COCCOCCNC(=O)COCCOCCNC(=O)CC[C@H](NC(=O)CCCCCCCCCCCCCCCCC(=O)O)C(=O)O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(=O)CCC(=O)N[C@H](CC(=O)O)C[N+](C)(C)C)C(=O)N[C@@H](CC(N)=O)C(O)N[C@@H](Cc3cccnc3)C(=O)N(C)CC(N)=O)CCOCC2)NC1=O] |
| | HOC18gEPEG2PEG2CO-r-Pen(3)-N-T-7MeW-K(Ac)-Pen(3)-AEF-2Nal-THP-K(d)-N-3Pya- |
| | Sar-CONH2 |
| 46 | 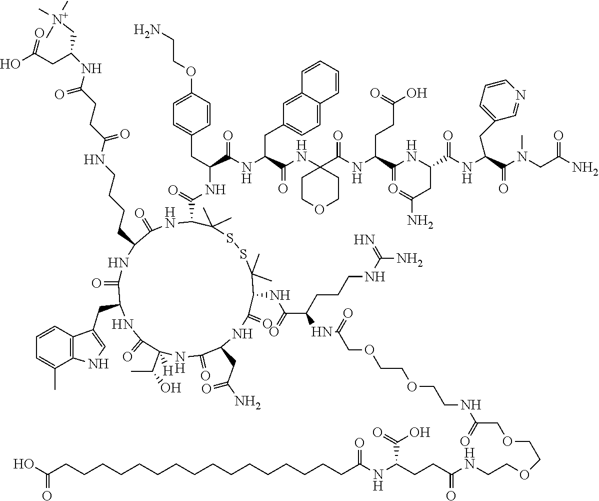 |
| | HOC18gEPEG2PEG2CO-r-Pen(3)-N-T-7MeW-K(d)-Pen(3)-AEF-2Nal-THP-E-N-3Pya-Sar- |
| | CONH2 |
| 47 | 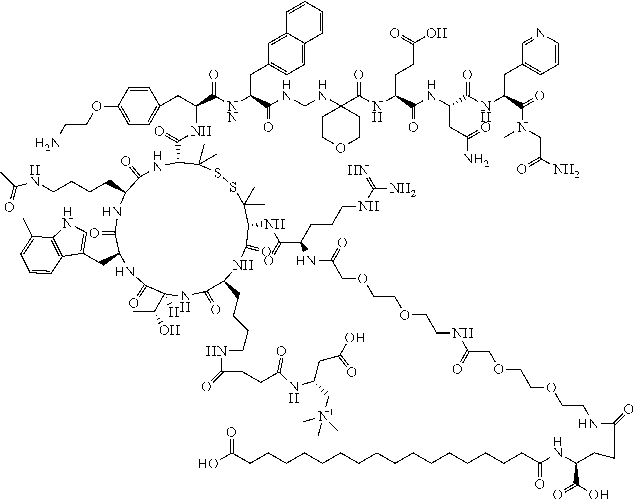 |
| | HOC18gEPEG2PEG2CO-r-Pen(3)-K(d)-T-7MeW-K(Ac)-Pen(3)-AEF-2Nal-THP-E-N-3Pya- |
| | Sar-CONH2 |
| 48 | 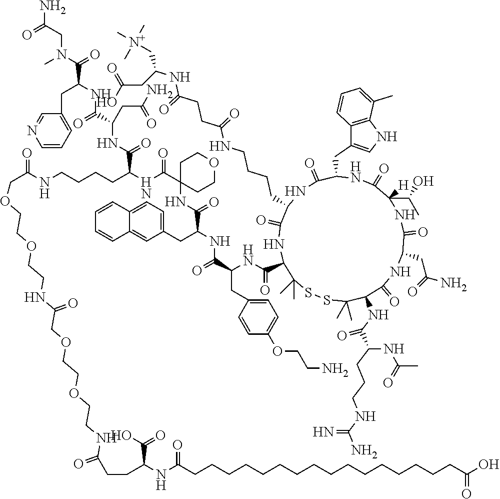 [SMILES: CC(=O)N[C@H](CCCNC(=N)N)C(=O)N[C@@H]1C(=O)N[C@@H](CC(N)=O)C(=O)N[C@@H]([C@@H](C)O)C(=O)N[C@@H](Cc2c[nH]c3c(C)cccc23)C(=O)N[C@@H](CCCCNC(=O)CCC(=O)N[C@H](CC(=O)O)C[N+](C)(C)C)C(=O)N[C@H](C(=O)N[C@@H](Cc2ccc(OCCN)cc2)C(=O)N[C@@H](Cc2ccc3ccccc3c2)C(=O)NC2(C(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(=O)N[C@@H](CC(N)=O)C(=O)N[C@@H](Cc3cccnc3)C(=O)N(C)CC(N)=O)CCOCC2)C(C)(C)SSC1(C)C] |
| | MeCO-r-Pen(3)-N-T-7MeW-K(d)-Pen(3)-AEF-2Nal-THP-K(PEG2PEG2gEC18OH)-N-3Pya- |
| | Sar-CONH2 |
| 49 | 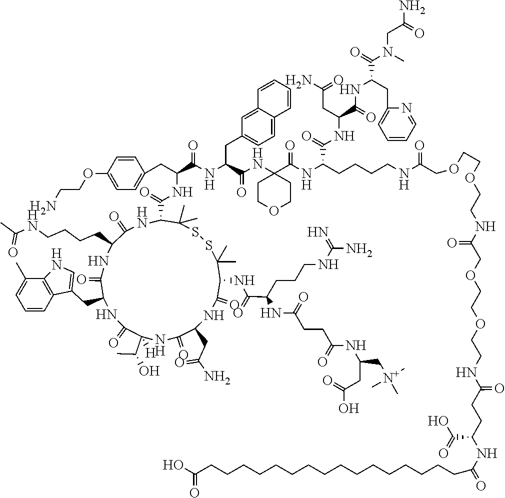 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(=O)CCC(=O)N[C@H](CC(=O)O)C[N+](C)(C)C)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(=O)N[C@@H](CC(N)=O)C(=O)N[C@@H](Cc3ccccn3)C(=O)N(C)CC(N)=O)CCOCC2)NC1=O] |
| | succiniccarn-r-Pen(3)-N-T-7MeW-K(Ac)-Pen(3)-AEF-2Nal-THP-K(PEG2PEG2gEC18OH)- |
| | N-3Pya-Sar-CONH2 |
| 50 | 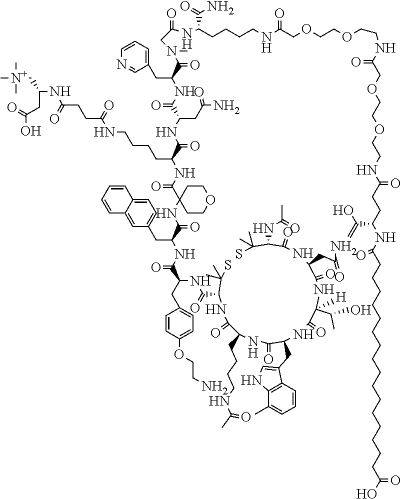 [SMILES: C=C(O)[C@H](CCC(=O)NCCOCCOCC(=O)NCCOCCOCC(=O)NCCCC[C@H](NC(=O)CN(C)C(=O)[C@H](Cc1cccnc1)NC(=O)[C@H](CC(N)=O)NC(=O)[C@H](CCCCNC(=O)CCC(=O)N[C@H](CC(=O)O)C[N+](C)(C)C)NC(=O)C1(NC(=O)[C@H](Cc2ccc3ccccc3c2)NC(=O)[C@H](Cc2ccc(OCCN)cc2)NC(=O)[C@H]2NC(=O)[C@H](CCCCNC(C)=O)NC(=O)[C@H](Cc3c[nH]c4c(C)cccc34)NC(=O)[C@H]([C@@H](C)O)NC(=O)[C@H](CC(N)=O)NC(=O)[C@@H](NC(C)=O)C(C)(C)SSC2(C)C)CCOCC1)C(N)=O)NC(=O)CCCCCCCCCCCCCCCCC(=O)O] |
| | MeCO-Pen(3)-N-T-7MeW-K(Ac)-Pen(3)-AEF-2Nal-THP-K(d)-N-3Pya-Sar- |
| | K(PEG2PEG2gEC18OH)-CONH2 |
| 51 | 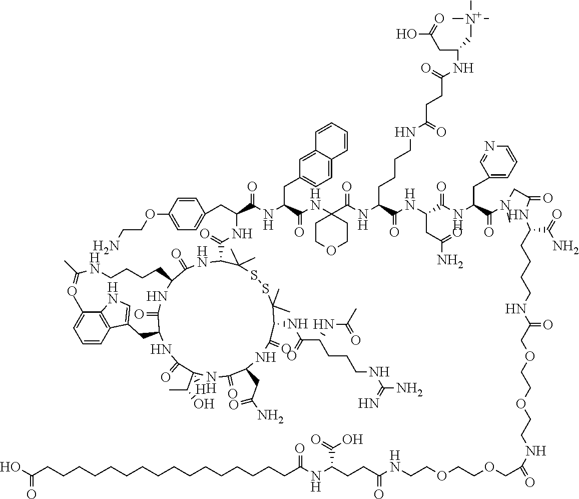 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(=O)CCC(=O)N[C@H](CC(=O)O)C[N+](C)(C)C)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)NC1=O] |
| | MeCO-r-Pen(3)-N-T-7MeW-K(Ac)-Pen(3)-AEF-2Nal-THP-K(d)-N-3Pya-Sar- |
| | K(PEG2PEG2gEC18OH)-CONH2 |
| 52 | 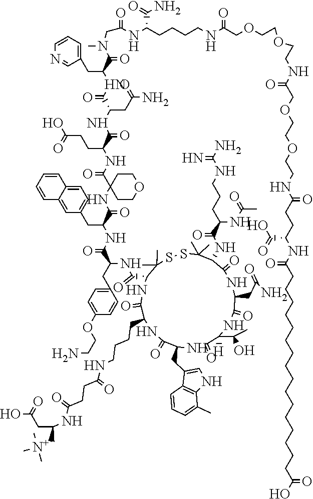 [SMILES: CC(=O)N[C@H](CCCNC(=N)N)C(=O)N[C@@H]1C(=O)N[C@@H](CC(N)=O)C(=O)N[C@@H]([C@@H](C)O)C(=O)N[C@@H](Cc2c[nH]c3c(C)cccc23)C(=O)N[C@@H](CCCCNC(=O)CCC(=O)N[C@H](CC(=O)O)C[N+](C)(C)C)C(=O)N[C@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)C(C)(C)SSC1(C)C] |
| | MeCO-r-Pen(3)-N-T-7MeW-K(d)-Pen(3)-AEF-2Nal-THP-E-N-3Pya-Sar- |
| | K(PEG2PEG2gEC18OH)-CONH2 |
| 53 | 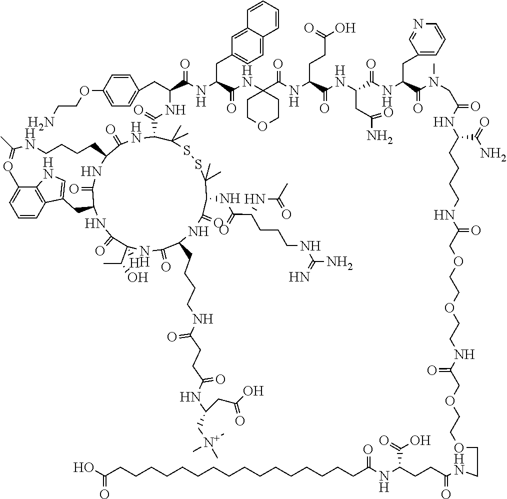 [SMILES: CC(=O)NCCCC[C@@H]1NC(=O)[C@H](Cc2c[nH]c3c(C)cccc23)NC(=O)[C@H]([C@@H](C)O)NC(=O)[C@H](CCCCNC(=O)CCC(=O)N[C@H](CC(=O)O)C[N+](C)(C)C)NC(=O)[C@@H](NC(=O)[C@@H](CCCNC(=N)N)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)NC1=O] |
| | MeCO-r-Pen(3)-K(d)-T-7MeW-K(Ac)-Pen(3)-AEF-2Nal-THP-E-N-3Pya-Sar- |
| | K(PEG2PEG2gEC18OH)-CONH2 |
| 54 | 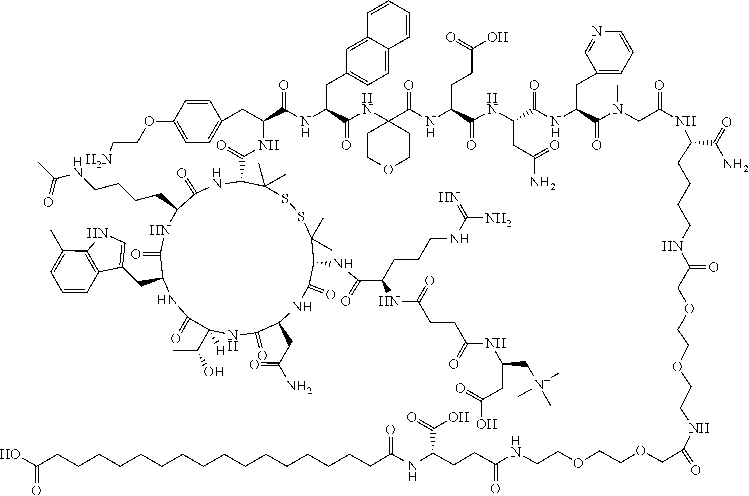 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(=O)CCC(=O)N[C@H](CC(=O)O)C[N+](C)(C)C)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)NC1=O] |
| | succiniccarn-r-Pen(3)-N-T-7MeW-K(Ac)-Pen(3)-AEF-2Nal-THP-E-N-3Pya-Sar- |
| | K(PEG2PEG2gEC18OH)-CONH2 |
| 55 | 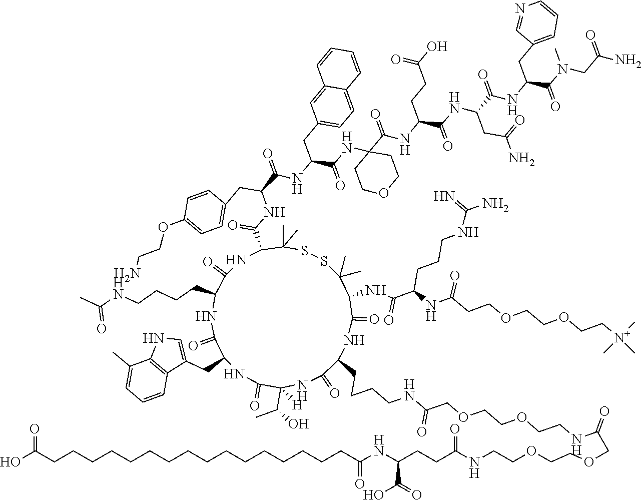 [SMILES: CC(=O)NCCCC[C@@H]1NC(=O)[C@H](Cc2c[nH]c3c(C)cccc23)NC(=O)[C@H]([C@@H](C)O)NC(=O)[C@H](CCCCNC(=O)COCCOCCNC(=O)COCCOCCNC(=O)CC[C@H](NC(=O)CCCCCCCCCCCCCCCCC(=O)O)C(=O)O)NC(=O)[C@@H](NC(=O)[C@@H](CCCNC(=N)N)NC(=O)CCOCCOCC[N+](C)(C)C)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O] |
| | cPEG3aCO-r-Pen(3)-K(PEG2PEG2gEC18OH)-T-7MeW-K(Ac)-Pen(3)-AEF-2Nal-THP-E- |
| | N-3Pya-Sar-CONH2 |
| 56 | 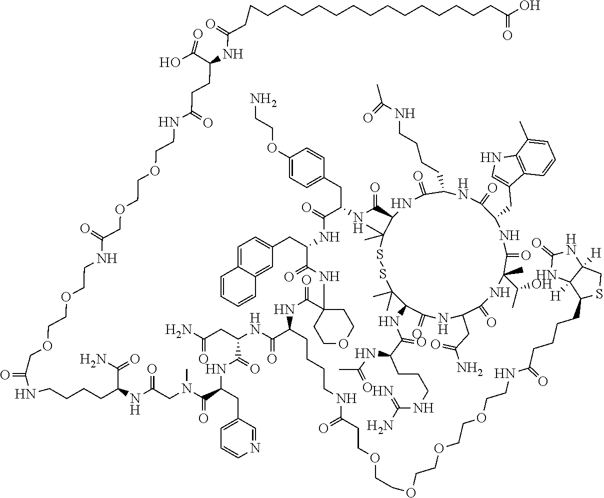 [SMILES: CC(=O)NCCCC[C@@H]1NC(=O)[C@H](Cc2c[nH]c3c(C)cccc23)NC(=O)[C@](C)([C@@H](C)O)NC(=O)C(CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(=O)CCOCCOCCOCCOCCNC(=O)CCCC[C@@H]3SC[C@@H]4NC(=O)N[C@@H]43)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)NC1=O] |
| | MeCO-r-Pen(3)-N-T-7MeW-K(Ac)-Pen(3)-AEF-2Nal-THP-K(BiotinPEG4)-N-3Pya-Sar- |
| | K(PEG2PEG2gEC18OH)-CONH2 |
| 57 | 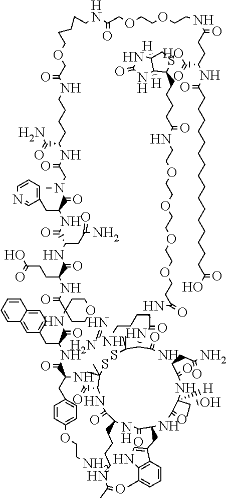 [SMILES: CC(=O)NCCCC[C@@H]1NC(=O)[C@@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(=O)CCOCCOCCOCCOCCNC(=O)CCCC[C@@H]2SC[C@@H]3NC(=O)N[C@@H]32)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCCCCNC(=O)COCCOCCNC(=O)CC[C@H](NC(=O)CCCCCCCCCCCCCCCCC(=O)O)C(=O)O)C(N)=O)CCOCC2)NC1=O] |
| | BiotinPEG4CO-r-Pen(3)-N-T-7MeW-K(Ac)-Pen(3)-AEF-2Nal-THP-E-N-3Pya-Sar- |
| | K(PEG2PEG2gEC18OH)-CONH2 |
| 58 | 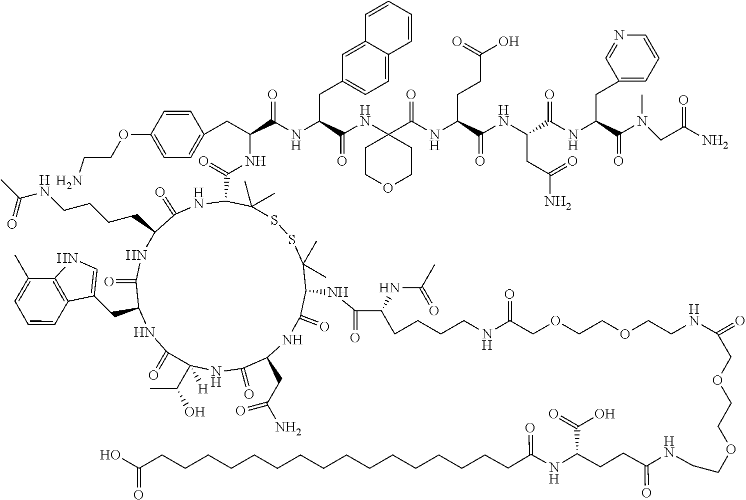 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCCNC(=O)COCCOCCNC(=O)COCCOCCNC(=O)CC[C@H](NC(=O)CCCCCCCCCCCCCCCCC(=O)O)C(=O)O)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O] |
| | MeCO-k(PEG2PEG2gEC18OH)-Pen(3)-N-T-7MeW-K(Ac)-Pen(3)-AEF-2Nal-THP-E-N- |
| | 3Pya-Sar-CONH2 |
| 59 | 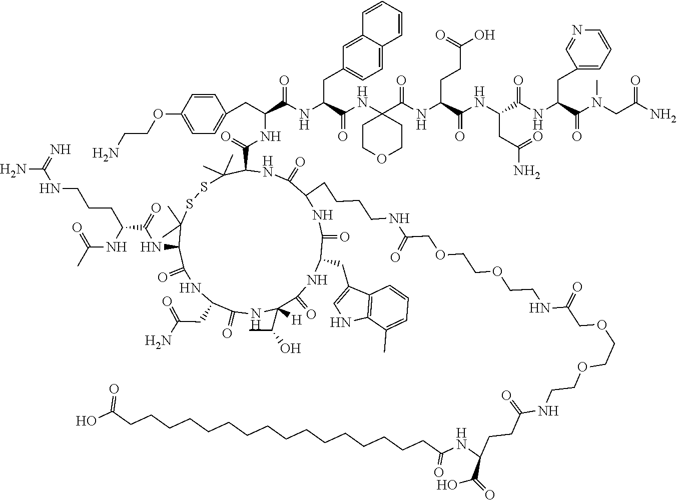 [SMILES: CC(=O)N[C@H](CCCNC(=N)N)C(=O)N[C@@H]1C(=O)N[C@@H](CC(N)=O)C(=O)N[C@@H]([C@@H](C)O)C(=O)N[C@@H](Cc2c[nH]c3c(C)cccc23)C(=O)NC(CCCCNC(=O)COCCOCCNC(=O)COCCOCCNC(=O)CC[C@H](NC(=O)CCCCCCCCCCCCCCCCC(=O)O)C(=O)O)C(=O)N[C@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)C(C)(C)SSC1(C)C] |
| | MeCO-r-Pen(3)-N-T-7MeW-K(PEG2PEG2gEC18OH)-Pen(3)-AEF-2Nal-THP-E-N-3Pya- |
| | Sar-CONH2 |
| 60 | 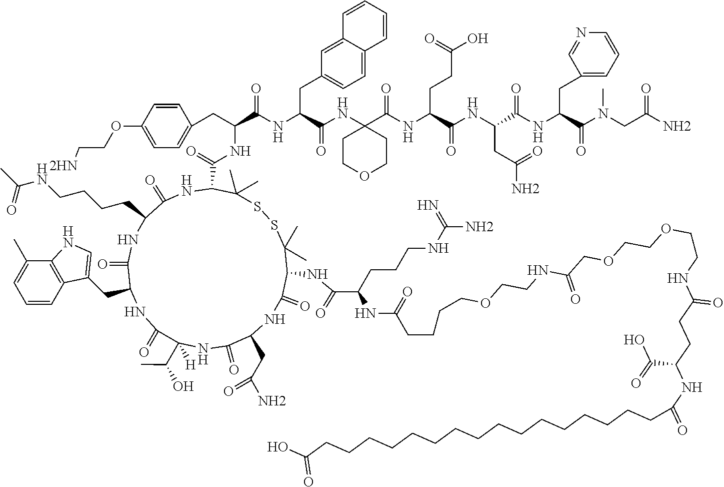 |
| | HOC18gEPEG2PEG2CO-r-Pen(3)-N-T-7MeW-K(Ac)-Pen(3)-AEF-2Nal-THP-E-N-3Pya- |
| | Sar-CONH2 |
| 61 | 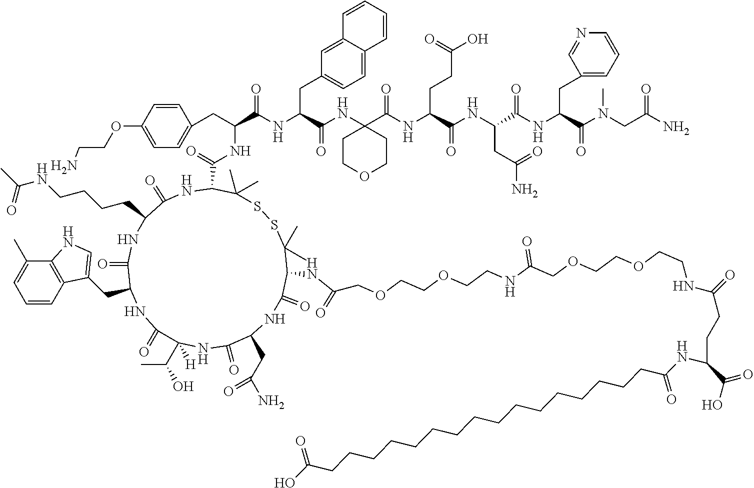 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)COCCOCCNC(=O)COCCOCCNC(=O)CC[C@H](NC(=O)CCCCCCCCCCCCCCCCC(=O)O)C(=O)O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O] |
| | HOC18gEPEG2PEG2CO-Pen(3)-N-T-7MeW-K(Ac)-Pen(3)-AEF-2Nal-THP-E-N-3Pya-Sar- |
| | CONH2 |
| 62 | 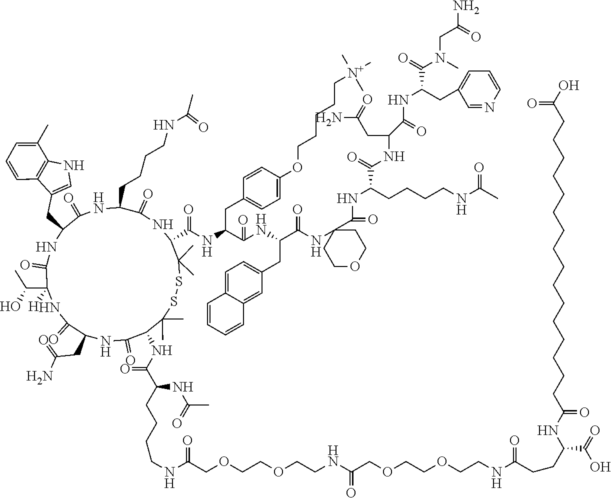 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@H](CCCCNC(=O)COCCOCCNC(=O)COCCOCCNC(=O)CC[C@H](NC(=O)CCCCCCCCCCCCCCCCC(=O)O)C(=O)O)NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(C)=O)C(=O)NC(CC(N)=O)C(=O)N[C@@H](Cc3cccnc3)C(=O)N(C)CC(N)=O)CCOCC2)NC1=O] |
| | MeCO-K(PEG2PEG2gEC18OH)-Pen(3)-N-T-7MeW-K(Ac)-Pen(3)-TMAPF-2Nal-THP- |
| | K(Ac)-3-Pya-Sar-CONH2 |
| 63 | 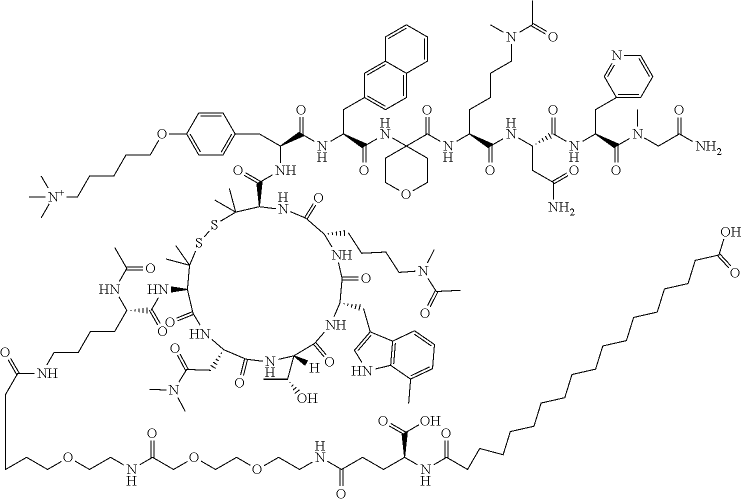 [SMILES: CC(=O)N[C@@H](CCCCNC(=O)CCCCOCCNC(=O)COCCOCCNC(=O)CC[C@H](NC(=O)CCCCCCCCCCCCCCCCC(=O)O)C(=O)O)C(=O)N[C@@H]1C(=O)N[C@@H](CC(=O)N(C)C)C(=O)N[C@@H]([C@@H](C)O)C(=O)N[C@@H](Cc2c[nH]c3c(C)cccc23)C(=O)N[C@@H](CCCCN(C)C(C)=O)C(=O)N[C@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(N)=O)CCOCC2)C(C)(C)SSC1(C)C] |
| | MeCO-K(PEG2PEG2gEC18OH)-Pen(3)-N(N(Me)2)-T-7MeW-K(NMeAc)-Pen(3)-TMAPF- |
| | 2Nal-THP-K(NMeAc)-N-3Pya-Sar-CONH2 |
| 64 | 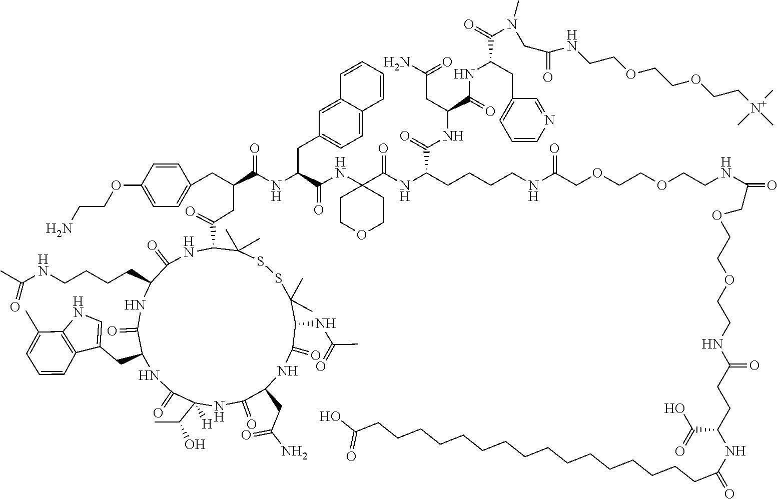 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)C[C@@H](Cc2ccc(OCCN)cc2)C(=O)N[C@@H](Cc2ccc3ccccc3c2)C(=O)NC2(C(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(=O)N[C@@H](CC(N)=O)C(=O)N[C@@H](Cc3cccnc3)C(=O)N(C)CC(=O)NCCOCCOCC[N+](C)(C)C)CCOCC2)NC1=O] |
| | MeCO-Pen(3)-N-T-7MeW-K(Ac)-Pen(3)-AEF-2Nal-THP-K(PEG2PEG2gEC18OH)-N-3Pya- |
| | Sar-CONH(PEG3a) |
| 65 | 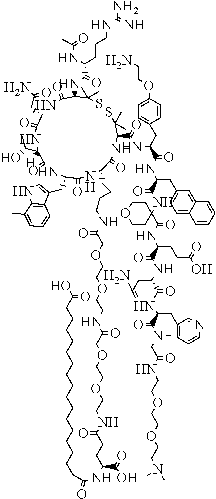 |
| | MeCO-r-Pen(3)-N-T-7MeW-K(PEG2PEG2gEC18OH)-Pen(3)-AEF-2Nal-THP-E-N-3Pya- |
| | Sar-CONH(PEG3a) |
| 66 | 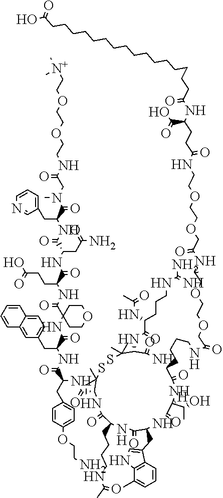 [SMILES: CC(=O)NCCCC[C@@H]1NC(=O)[C@H](Cc2c[nH]c3c(C)cccc23)NC(=O)[C@H]([C@@H](C)O)NC(=O)[C@H](CCCCNC(=O)COCCOCCNC(=O)COCCOCCNC(=O)CC[C@H](NC(=O)CCCCCCCCCCCCCCCCC(=O)O)C(=O)O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)NCCOCCOCC[N+](C)(C)C)CCOCC2)NC1=O] |
| | MeCO-r-Pen(3)-K(PEG2PEG2gEC18OH)-T-7MeW-K(Ac)-Pen(3)-AEF-2Nal-THP-E-N- |
| | 3Pya-Sar-CONH(PEG3a) |
| 67 | 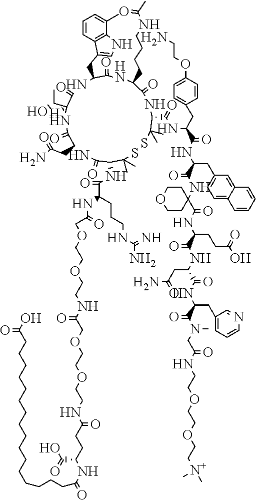 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(=O)COCCOCCNC(=O)COCCOCCNC(=O)CC[C@H](NC(=O)CCCCCCCCCCCCCCCCC(=O)O)C(=O)O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)NCCOCCOCC[N+](C)(C)C)CCOCC2)NC1=O] |
| | HOC18gEPEG2PEG2CO-r-Pen(3)-N-T-7MeW-K(Ac)-Pen(3)-AEF-2Nal-THP-E-N-3Pya- |
| | Sar-CONH(PEG3a) |
| 68 | 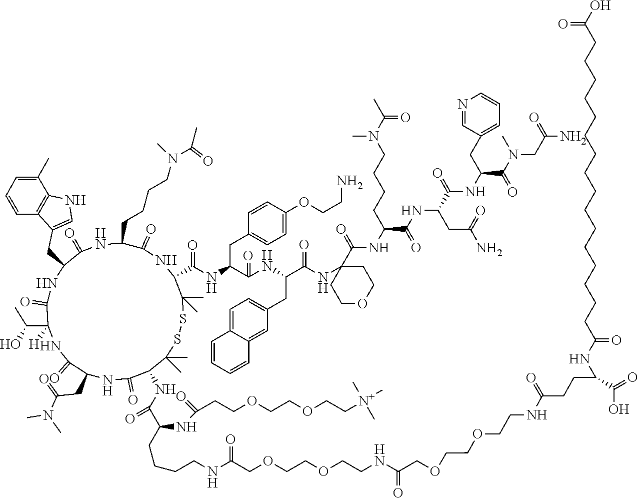 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)[C@H](CCCCNC(=O)COCCOCCNC(=O)COCCOCCNC(=O)CC[C@H](NC(=O)CCCCCCCCCCCCCCCCC(=O)O)C(=O)O)NC(=O)CCOCCOCC[N+](C)(C)C)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(N)=O)CCOCC2)NC1=O] |
| | cPEG3aCO-K(PEG2PEG2gEC18OH)-Pen(3)-N(N(Me)2)-T-7MeW-K(NMeAc)-Pen(3)-AEF- |
| | 2Nal-THP-K(NMeAc)-N-3Pya-Sar-CONH2 |
| 69 | 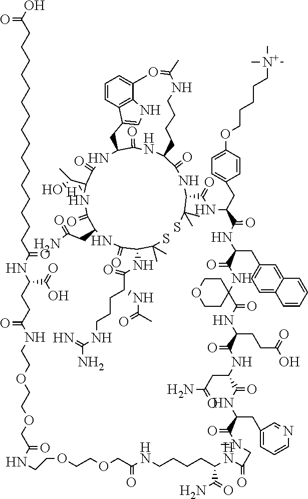 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)NC1=O] |
| | MeCO-r-Pen(3)-N-T-7MeW-K(Ac)-Pen(3)-TMAPF-2Nal-THP-E-N-3Pya-Sar- |
| | K(PEG2PEG2C18OH)-CONH2 |
| 70 | 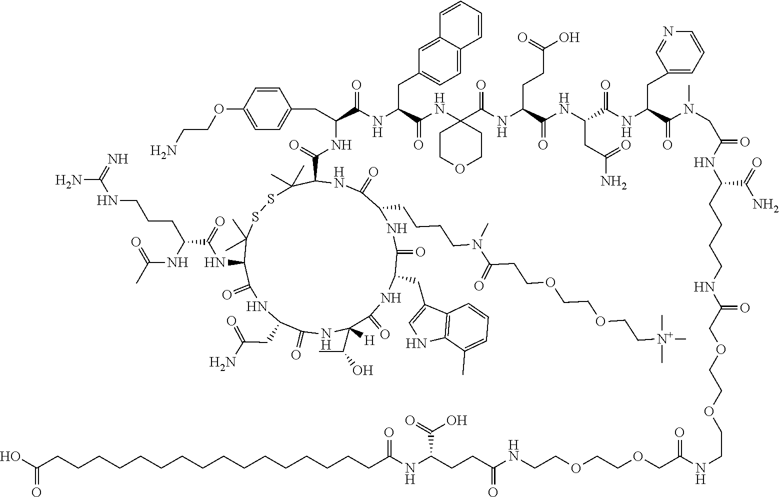 [SMILES: CC(=O)N[C@H](CCCNC(=N)N)C(=O)N[C@@H]1C(=O)N[C@@H](CC(N)=O)C(=O)N[C@@H]([C@@H](C)O)C(=O)N[C@@H](Cc2c[nH]c3c(C)cccc23)C(=O)N[C@@H](CCCCN(C)C(=O)CCOCCOCC[N+](C)(C)C)C(=O)N[C@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)C(C)(C)SSC1(C)C] |
| | MeCO-r-Pen(3)-N-T-7MeW-K(NMecPEG3a)-Pen(3)-AEF-2Nal-THP-E-N-3Pya-Sar- |
| | K(PEG2PEG2C18OH)-CONH2 |
| 71 | 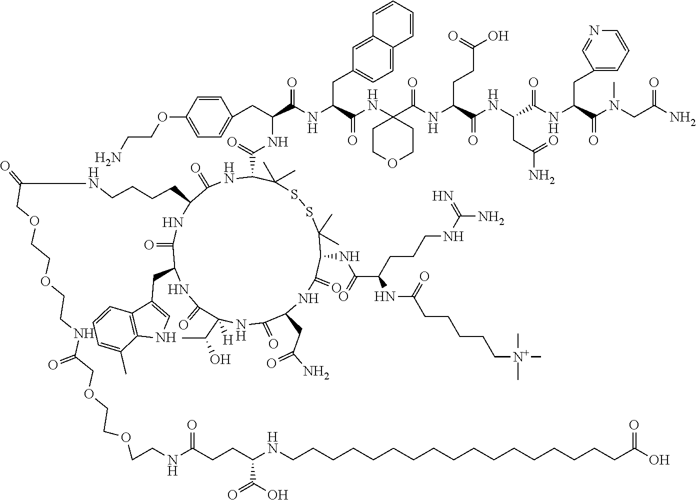 [SMILES: Cc1cccc2c(C[C@@H]3NC(=O)[C@H]([C@@H](C)O)NC(=O)[C@H](CC(N)=O)NC(=O)[C@@H](NC(=O)[C@@H](CCCNC(=N)N)NC(=O)CCCCC[N+](C)(C)C)C(C)(C)SSC(C)(C)[C@@H](C(=O)N[C@@H](Cc4ccc(OCCN)cc4)C(=O)N[C@@H](Cc4ccc5ccccc5c4)C(=O)NC4(C(=O)N[C@@H](CCC(=O)O)C(=O)N[C@@H](CC(N)=O)C(=O)N[C@@H](Cc5cccnc5)C(=O)N(C)CC(N)=O)CCOCC4)NC(=O)[C@H](CCCCNC(=O)COCCOCCNC(=O)COCCOCCNC(=O)CC[C@H](NCCCCCCCCCCCCCCCCCC(=O)O)C(=O)O)NC3=O)c[nH]c12] |
| | 5cpaCO-r-Pen(3)-N-T-7MeW-K(PEG2PEG2C18OH)-Pen(3)-AEF-2Nal-THP-E-N-3Pya- |
| | Sar-CONH2 |
| 72 | 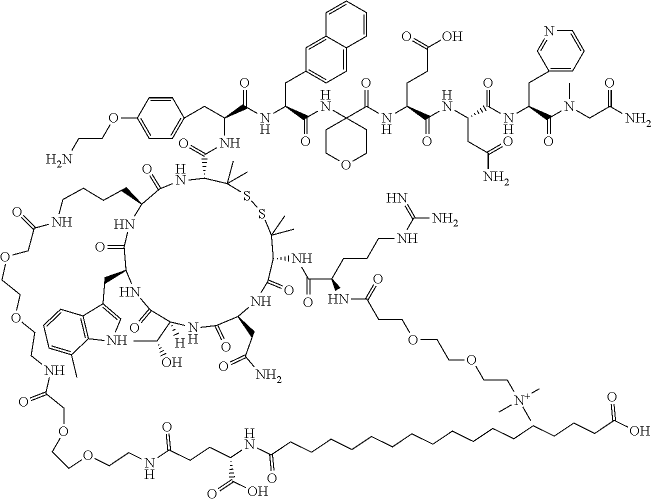 [SMILES: Cc1cccc2c(C[C@@H]3NC(=O)[C@H]([C@@H](C)O)NC(=O)[C@H](CC(N)=O)NC(=O)[C@@H](NC(=O)[C@@H](CCCNC(=N)N)NC(=O)CCOCCOCC[N+](C)(C)C)C(C)(C)SSC(C)(C)[C@@H](C(=O)N[C@@H](Cc4ccc(OCCN)cc4)C(=O)N[C@@H](Cc4ccc5ccccc5c4)C(=O)NC4(C(=O)N[C@@H](CCC(=O)O)C(=O)N[C@@H](CC(N)=O)C(=O)N[C@@H](Cc5cccnc5)C(=O)N(C)CC(N)=O)CCOCC4)NC(=O)[C@H](CCCCNC(=O)COCCOCCNC(=O)COCCOCCNC(=O)CC[C@H](NC(=O)CCCCCCCCCCCCCCCCC(=O)O)C(=O)O)NC3=O)c[nH]c12] |
| | cPEG3aCO-r-Pen(3)-N-T-7MeW-K(PEG2PEG2C18OH)-Pen(3)-AEF-2Nal-THP-E-N-3Pya- |
| | Sar-CONH2 |
| 73 | 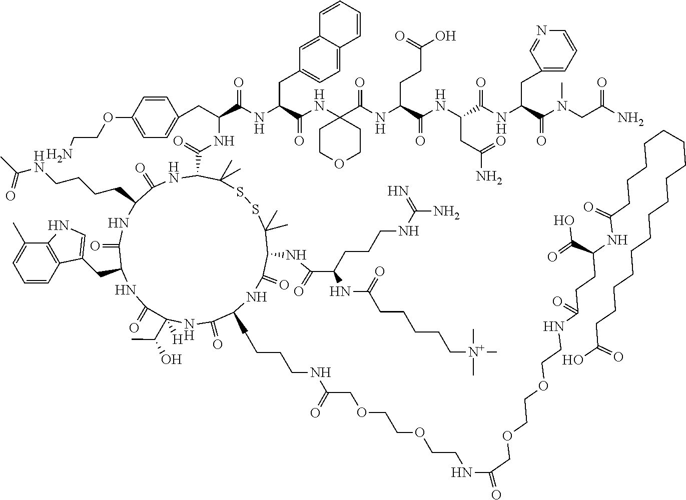 [SMILES: CC(=O)NCCCC[C@@H]1NC(=O)[C@H](Cc2c[nH]c3c(C)cccc23)NC(=O)[C@H]([C@@H](C)O)NC(=O)[C@H](CCCCNC(=O)COCCOCCNC(=O)COCCOCCNC(=O)CC[C@H](NC(=O)CCCCCCCCCCCCCCCCC(=O)O)C(=O)O)NC(=O)[C@@H](NC(=O)[C@@H](CCCNC(=N)N)NC(=O)CCCCC[N+](C)(C)C)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O] |
| | 5cpaCO-r-Pen(3)-K(PEG2PEG2gEC18OH)-T-7MeW-K(Ac)-Pen(3)-AEF-2Nal-THP-E-N- |
| | 3Pya-Sar-CONH2 |
| 74 | 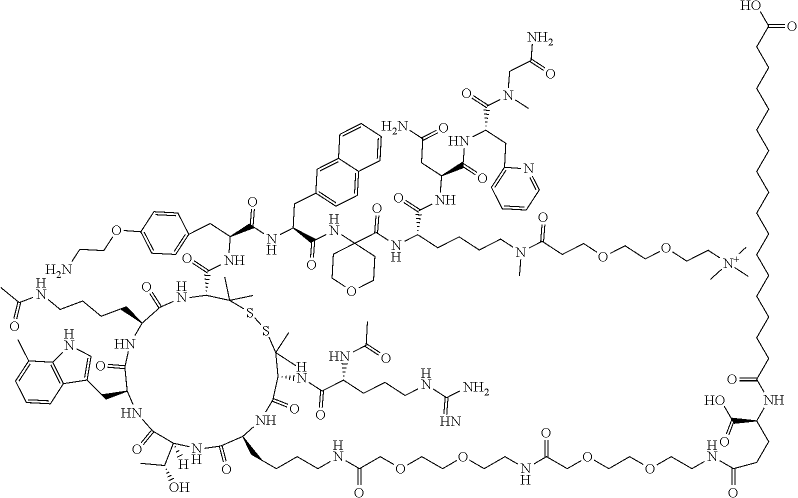 [SMILES: CC(=O)NCCCC[C@@H]1NC(=O)[C@H](Cc2c[nH]c3c(C)cccc23)NC(=O)[C@H]([C@@H](C)O)NC(=O)[C@H](CCCCNC(=O)COCCOCCNC(=O)COCCOCCNC(=O)CC[C@H](NC(=O)CCCCCCCCCCCCCCCCC(=O)O)C(=O)O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCN(C)C(=O)CCOCCOCC[N+](C)(C)C)C(=O)N[C@@H](CC(N)=O)C(=O)N[C@@H](Cc3ccccn3)C(=O)N(C)CC(N)=O)CCOCC2)NC1=O] |
| | MeCO-r-Pen(3)-K(PEG2PEG2gEC18OH)-T-7MeW-K(Ac)-Pen(3)-AEF-2Nal-THP- |
| | K(NMecPEG3a)-N-3Pya-Sar-CONH2Â |
| 75 | 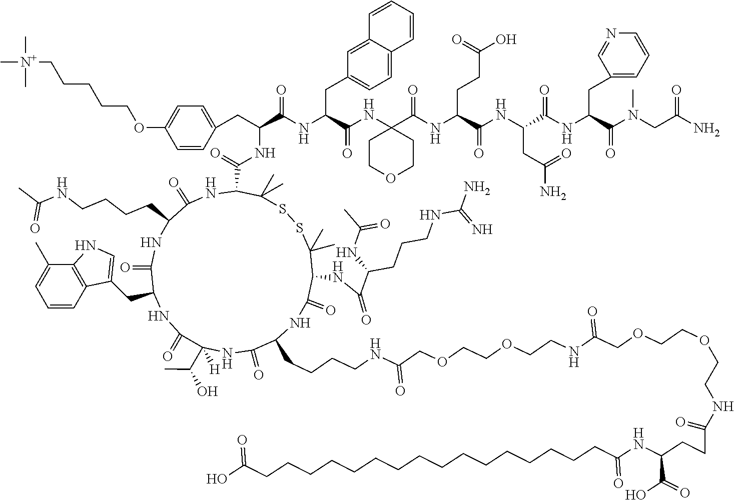 [SMILES: CC(=O)NCCCC[C@@H]1NC(=O)[C@H](Cc2c[nH]c3c(C)cccc23)NC(=O)[C@H]([C@@H](C)O)NC(=O)[C@H](CCCCNC(=O)COCCOCCNC(=O)COCCOCCNC(=O)CC[C@H](NC(=O)CCCCCCCCCCCCCCCCC(=O)O)C(=O)O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O] |
| | MeCO-r-Pen(3)-K(PEG2PEG2gEC18OH)-T-7MeW-K(Ac)-Pen(3)-TMAPF-2Nal-THP-E-N- |
| | 3Pya-Sar-CONH2 |
| 76 | 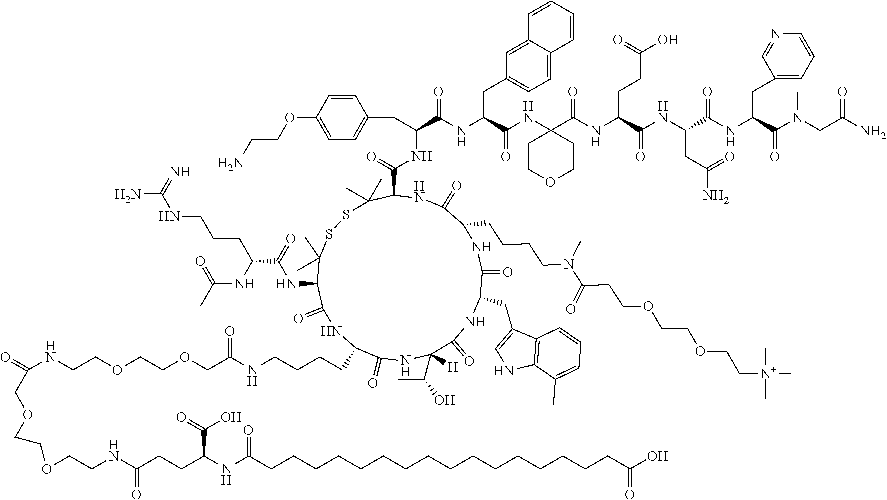 [SMILES: CC(=O)N[C@H](CCCNC(=N)N)C(=O)N[C@@H]1C(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(=O)N[C@@H]([C@@H](C)O)C(=O)N[C@@H](Cc2c[nH]c3c(C)cccc23)C(=O)N[C@@H](CCCCN(C)C(=O)CCOCCOCC[N+](C)(C)C)C(=O)N[C@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)C(C)(C)SSC1(C)C] |
| | MeCO-r-Pen(3)-K(PEG2PEG2gEC18OH)-T-7MeW-K(NMecPEG3a)-Pen(3)-AEF-2Nal- |
| | THP-E-N-3Pya-Sar-CONH2Â |
| 77 | 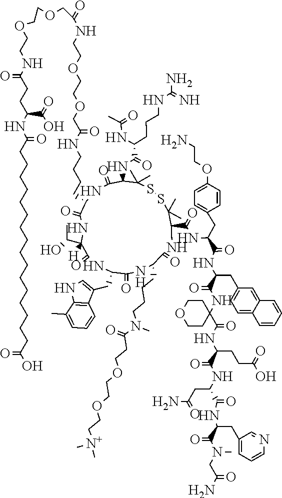 [SMILES: CC(=O)N[C@H](CCCNC(=N)N)C(=O)N[C@@H]1C(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(=O)N[C@@H]([C@@H](C)O)C(=O)N[C@@H](Cc2c[nH]c3c(C)cccc23)C(=O)N[C@@H](CCCCN(C)C(=O)CCOCCOCC[N+](C)(C)C)C(=O)N[C@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)C(C)(C)SSC1(C)C] |
| | 5cpaCO-Pen(3)-N-T-7MeW-K(Ac)-Pen(3)-AEF-2Nal-THP-K(PEG2PEG2gEC18OH)-N- |
| | 3Pya-Sar-CONH2 |
| 78 | 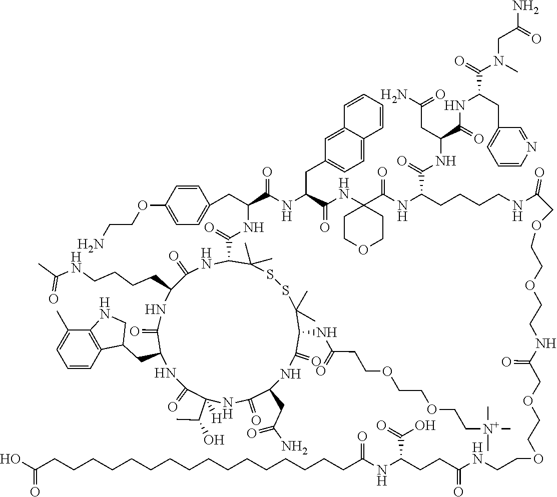 [SMILES: CC(=O)NCCCC[C@@H]1NC(=O)[C@H](CC2CNc3c(C)cccc32)NC(=O)[C@H]([C@@H](C)O)NC(=O)[C@H](CC(N)=O)NC(=O)[C@@H](NC(=O)CCOCCOCC[N+](C)(C)C)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(=O)N[C@@H](CC(N)=O)C(=O)N[C@@H](Cc3cccnc3)C(=O)N(C)CC(N)=O)CCOCC2)NC1=O] |
| | cPEG3aCO-Pen(3)-N-T-7MeW-K(Ac)-Pen(3)-AEF-2Nal-THP-K(PEG2PEG2gEC18OH)-N- |
| | 3Pya-Sar-CONH2 |
| 79 | 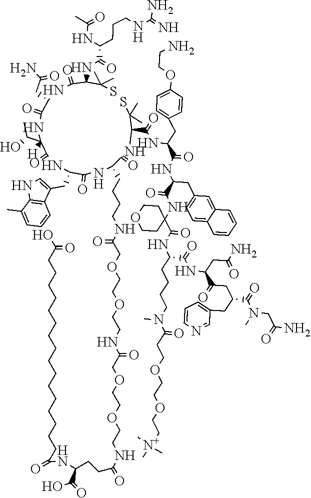 [SMILES: CC(=O)N[C@H](CCCNC(=N)N)C(=O)N[C@@H]1C(=O)N[C@@H](CC(N)=O)C(=O)N[C@@H]([C@@H](C)O)C(=O)N[C@@H](Cc2c[nH]c3c(C)cccc23)C(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(=O)N[C@H](C(=O)N[C@@H](Cc2ccc(OCCN)cc2)C(=O)N[C@@H](Cc2ccc3ccccc3c2)C(=O)NC2(C(=O)N[C@@H](CCCCN(C)C(=O)CCOCCOCC[N+](C)(C)C)C(=O)N[C@@H](CC(N)=O)C(=O)C[C@@H](Cc3cccnc3)C(=O)N(C)CC(N)=O)CCOCC2)C(C)(C)SSC1(C)C] |
| | MeCO-r-Pen(3)-N-T-7MeW-K(PEG2PEG2gEC18OH)-Pen(3)-AEF-2Nal-THP- |
| | K(NMecPEG3a)-N-3Pya-Sar-CONH2 |
| 80 | 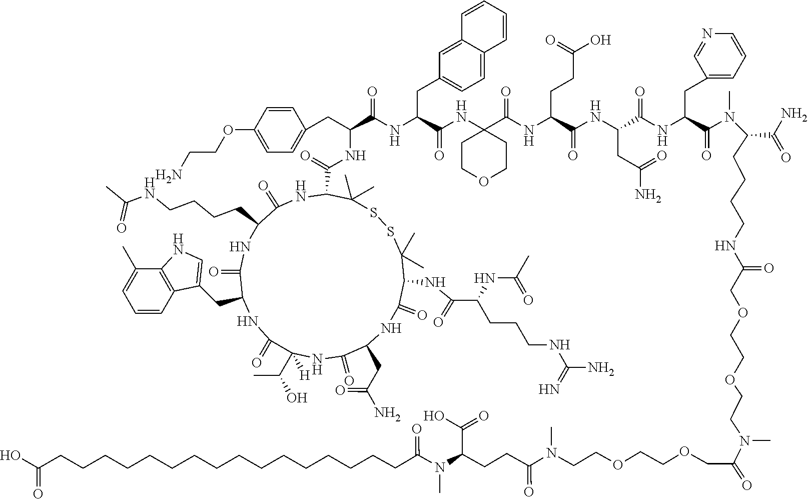 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)[C@@H](CCCCNC(=O)COCCOCCN(C)C(=O)COCCOCCN(C)C(=O)CC[C@H](C(=O)O)N(C)C(=O)CCCCCCCCCCCCCCCCC(=O)O)C(N)=O)CCOCC2)NC1=O] |
| | MeCO-r-Pen(3)-N-T-7MeW-K(Ac)-Pen(3)-AEF-2Nal-THP-E-N-3Pya- |
| | NMeK(PEG2NMePEG2NMegENMeC18OH)-CONH2 |
| 81 | 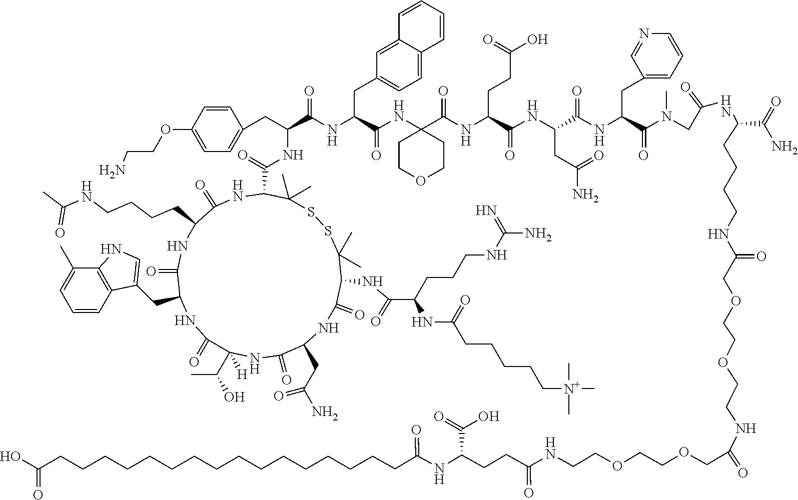 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(=O)CCCCC[N+](C)(C)C)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)NC1=O] |
| | 5cpaCO-r-Pen(3)-N-T-7MeW-K(Ac)-Pen(3)-AEF-2Nal-THP-E-N-3Pya-Sar- |
| | K(PEG2PEG2gEC18OH)-CONH2 |
| 82 | 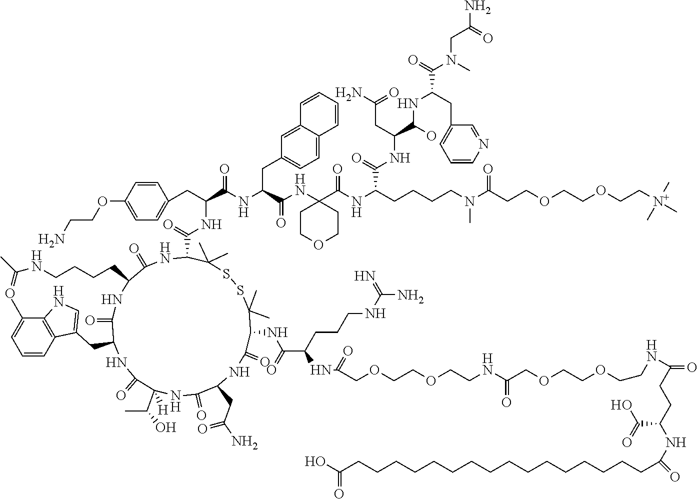 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(=O)COCCOCCNC(=O)COCCOCCNC(=O)CC[C@H](NC(=O)CCCCCCCCCCCCCCCCC(=O)O)C(=O)O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCN(C)C(=O)CCOCCOCC[N+](C)(C)C)C(=O)N[C@@H](CC(N)=O)C(O)N[C@@H](Cc3cccnc3)C(=O)N(C)CC(N)=O)CCOCC2)NC1=O] |
| | HOC18gEPEG2PEG2CO-r-Pen(3)-N-T-7MeW-K(Ac)-Pen(3)-AEF-2Nal-THP- |
| | K(NMecPEG3a)-N-3Pya-Sar-CONH2 |
| 83 | 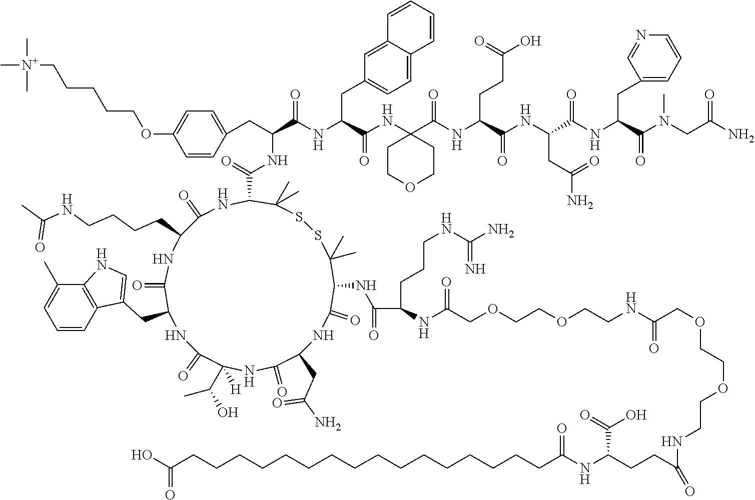 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(=O)COCCOCCNC(=O)COCCOCCNC(=O)CC[C@H](NC(=O)CCCCCCCCCCCCCCCCC(=O)O)C(=O)O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O] |
| | HOC18gEPEG2PEG2CO-r-Pen(3)-N-T-7MeW-K(Ac)-Pen(3)-TMAPF-2Nal-THP-E-N-3Pya- |
| | Sar-CONH2 |
| 84 | 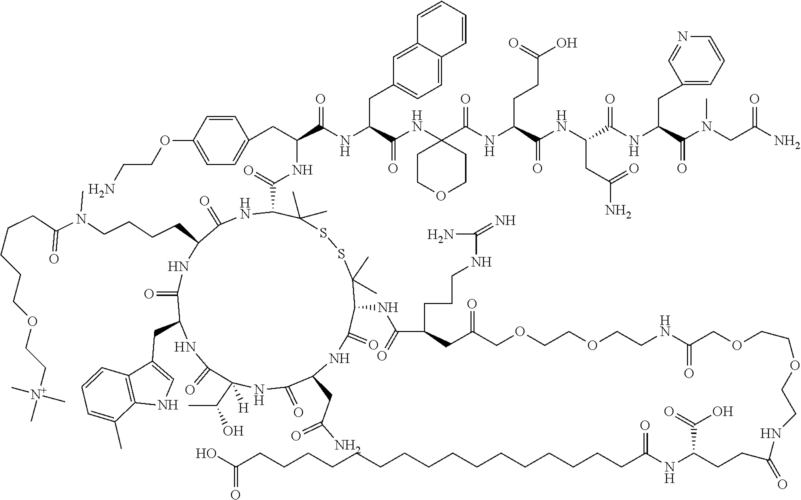 [SMILES: Cc1cccc2c(C[C@@H]3NC(=O)[C@H]([C@@H](C)O)NC(=O)[C@H](CC(N)=O)NC(=O)[C@@H](NC(=O)[C@@H](CCCNC(=N)N)CC(=O)COCCOCCNC(=O)COCCOCCNC(=O)CC[C@H](NC(=O)CCCCCCCCCCCCCCCCC(=O)O)C(=O)O)C(C)(C)SSC(C)(C)[C@@H](C(=O)N[C@@H](Cc4ccc(OCCN)cc4)C(=O)N[C@@H](Cc4ccc5ccccc5c4)C(=O)NC4(C(=O)N[C@@H](CCC(=O)O)C(=O)N[C@@H](CC(N)=O)C(=O)N[C@@H](Cc5cccnc5)C(=O)N(C)CC(N)=O)CCOCC4)NC(=O)[C@H](CCCCN(C)C(=O)CCCCCOCC[N+](C)(C)C)NC3=O)c[nH]c12] |
| | HOC18gEPEG2PEG2CO-r-Pen(3)-N-T-7MeW-K(NMecPEG3a)-Pen(3)-AEF-2Nal-THP-E- |
| | N-3Pya-Sar-CONH2 |
| 85 | 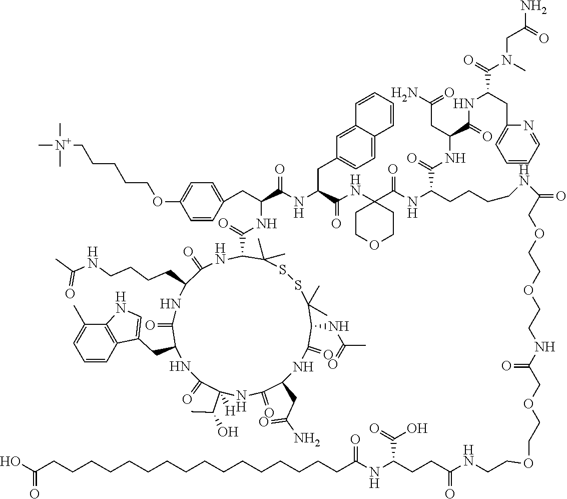 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(=O)N[C@@H](CC(N)=O)C(=O)N[C@@H](Cc3ccccn3)C(=O)N(C)CC(N)=O)CCOCC2)NC1=O] |
| | MeCO-Pen(3)-N-T-7MeW-K(Ac)-Pen(3)-TMAPF-2Nal-THP-K(PEG2PEG2gEC18OH)-N- |
| | 3Pya-Sar-CONH2 |
| 86 | 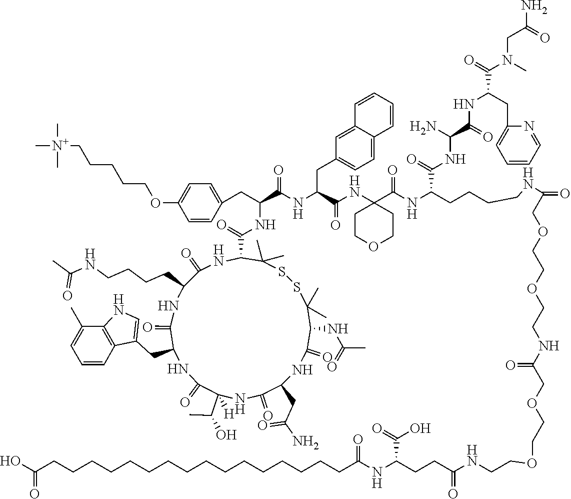 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(=O)N[C@@H](N)C(=O)N[C@@H](Cc3ccccn3)C(=O)N(C)CC(N)=O)CCOCC2)NC1=O] |
| | MeCO-Pen(3)-N-T-7MeW-K(NMecPEG3a)-Pen(3)-AEF-2Nal-THP- |
| | K(PEG2PEG2gEC18OH)-N-3Pya-Sar-CONH2 |
| 87 | 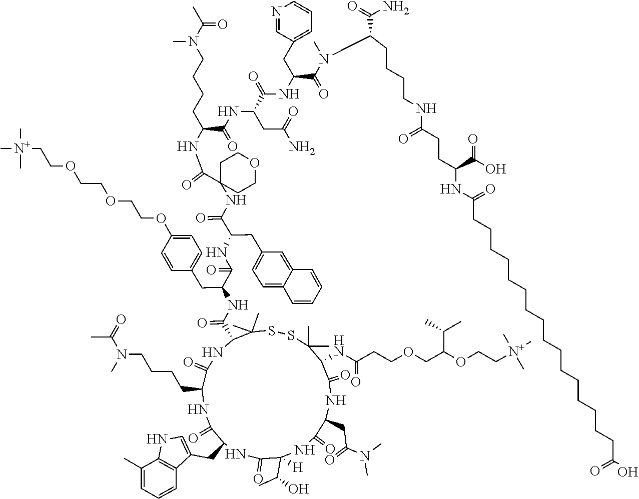 |
| | cPEG3aCO-Pen(3)-(N(N(Me)2)-T-7MeW-K(NMeAc)-Pen(3)-APEG3F-2Nal-THP- |
| | K(NMeAc)-N-3Pya-NMeK(gEC18OH)-CONH2 |
| 88 | 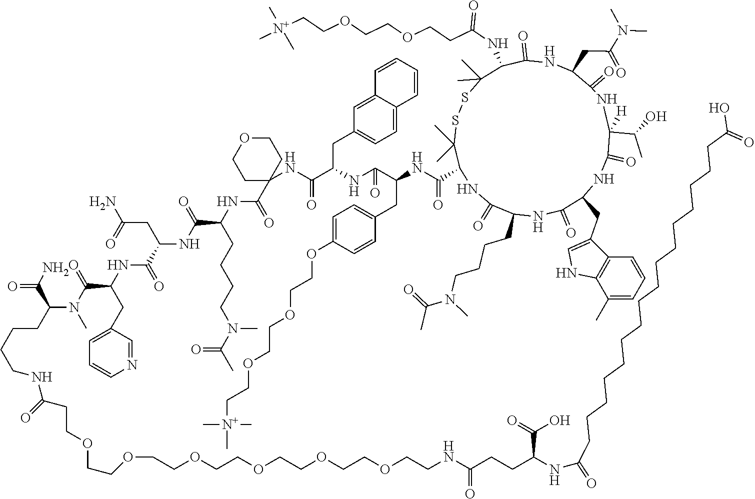 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)CCOCCOCC[N+](C)(C)C)C(C)(C)SSC(C)(C)[C@@H](C(=O)N[C@@H](Cc2ccc(OCCOCCOCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)[C@@H](CCCCNC(=O)CCOCCOCCOCCOCCOCCOCCNC(=O)CC[C@H](NC(=O)CCCCCCCCCCCCCCCCC(=O)O)C(=O)O)C(N)=O)CCOCC2)NC1=O] |
| | cPEG3aCO-Pen(3)-N(N(Me)2)-T-7MeW-K(NMeAc)-Pen(3)-APEG3F-2Nal-THP- |
| | K(NMeAc)-N-3Pya-NMeK(PEG6gEC18OH)-CONH2 |
| 89 | 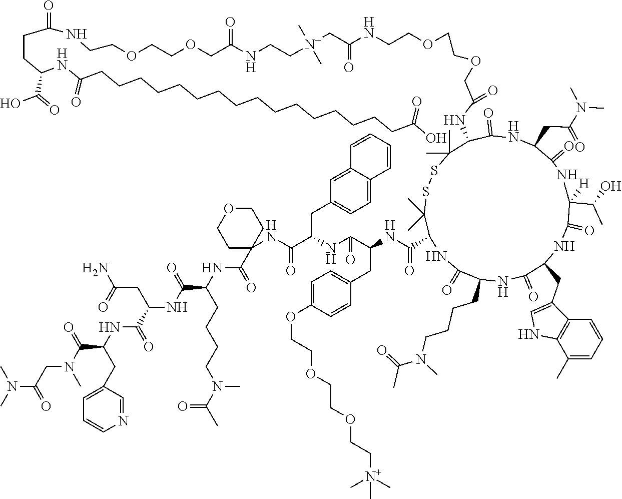 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)COCCOCCNC(=O)C[N+](C)(C)CCNC(=O)COCCOCCNC(=O)CC[C@H](NC(=O)CCCCCCCCCCCCCCCCC(=O)O)C(=O)O)C(C)(C)SSC(C)(C)[C@@H](C(=O)N[C@@H](Cc2ccc(OCCOCCOCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] |
| | HOC18gEPEG2SP6PEG2CO-Pen(3)-N(N(Me)2)-T-7MeW-K(NMeAc)-Pen(3)-APEG3F- |
| | 2Nal-THP-K(NMeAc)-N-3Pya-Sar-CON(Me)2 |
| 90 | 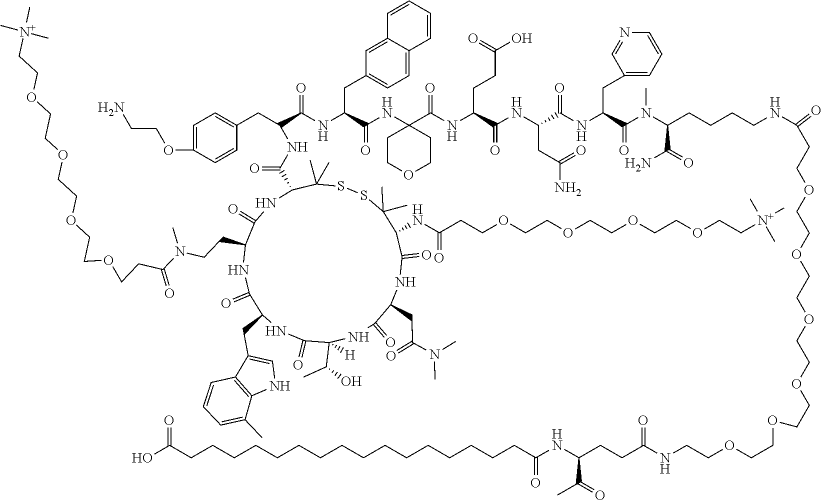 |
| | cPEG5aCO-Pen(3)-N(N(Me)2)-T-7MeW-Dab(NMecPEG5a)-Pen(3)-AEF-2Nal-THP-E-N- |
| | 3Pya-NMeK(PEG6gEC18OH)-CONH2 |
| 91 | 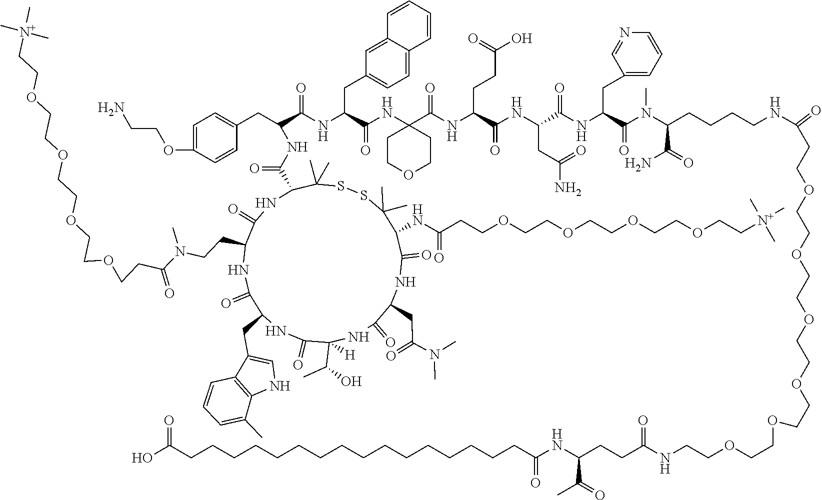 |
| | HOC18gEPEG2PEG2COSP6-Pen(3)-N(N(Me)2)-T-7MeW-K(NMeAc)-Pen(3)-APEG3F- |
| | 2Nal-THP-K(NMeAc)-N-3Pya-Sar-CON(Me)2 |
| 92 | 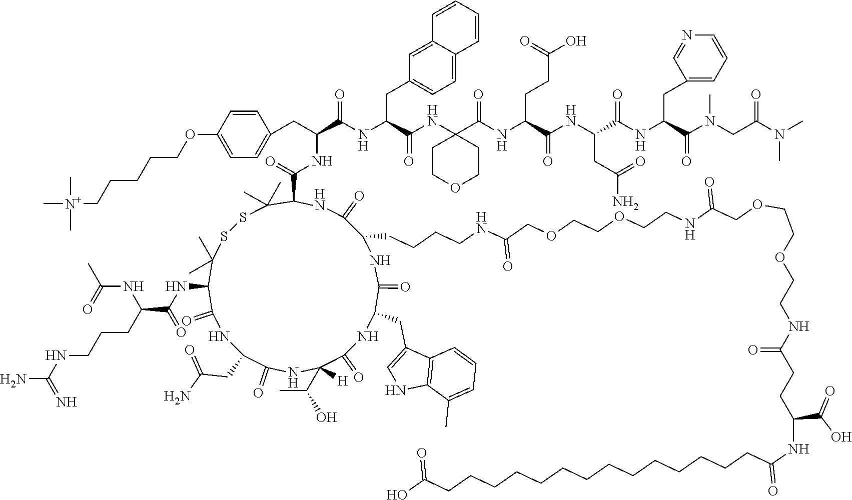 [SMILES: CC(=O)N[C@H](CCCNC(=N)N)C(=O)N[C@@H]1C(=O)N[C@@H](CC(N)=O)C(=O)N[C@@H]([C@@H](C)O)C(=O)N[C@@H](Cc2c[nH]c3c(C)cccc23)C(=O)N[C@@H](CCCCNC(=O)COCCOCCNC(=O)COCCOCCNC(=O)CC[C@H](NC(=O)CCCCCCCCCCCCCCC(=O)O)C(=O)O)C(=O)N[C@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N(C)C)CCOCC2)C(C)(C)SSC1(C)C] |
| | MeCO-r-Pen(3)-N-T-7MeW-K(PEG2PEG2gEC18OH)-Pen(3)-TMAPF-2Nal-THF-E-N- |
| | 3Pya-Sar-CONH2 |
| 93 | 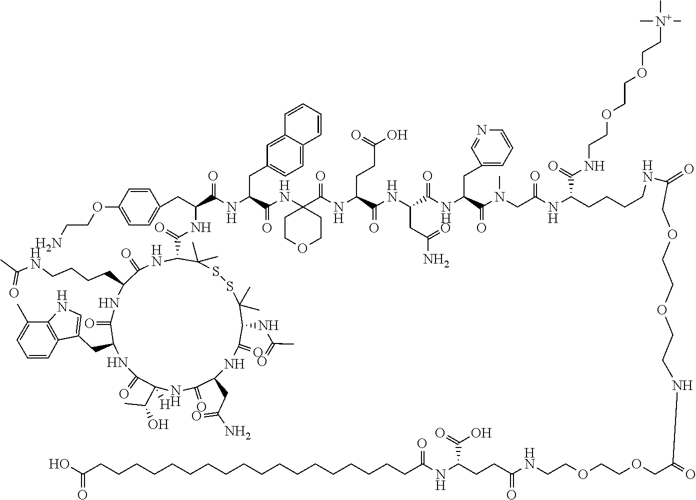 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCCCC(=O)O)C(=O)O)C(=O)NCCOCCOCC[N+](C)(C)C)CCOCC2)NC1=O] |
| | MeCO-Pen(3)-N-T-7MeW-K(Ac)-Pen(3)-AEF-2Nal-THP-K(Ac)-N-3Pya-Sar- |
| | K(PEG2PEG2gEC20OH)-CONH(PEG3a) |
| 94 | 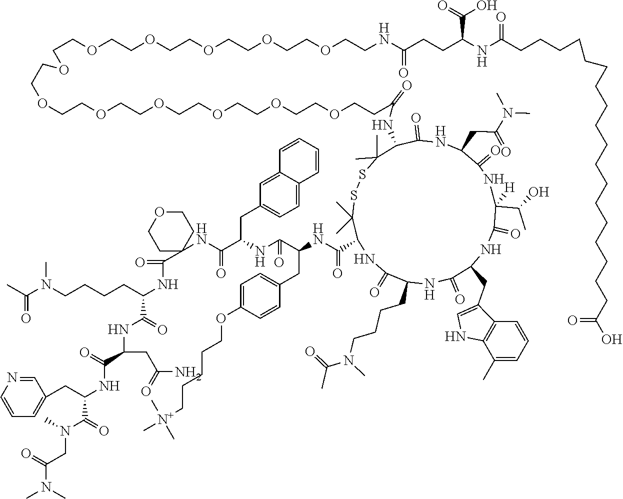 |
| | HOC20gEPEG12CO-Pen(3)-N(N(Me)2)-T-7MeW-K(NMeAc)-Pen(3)-TMAPF-2Nal-THP- |
| | K(NMeAc)-N-3Pya-Sar-CON(Me)2 |
| 95 | 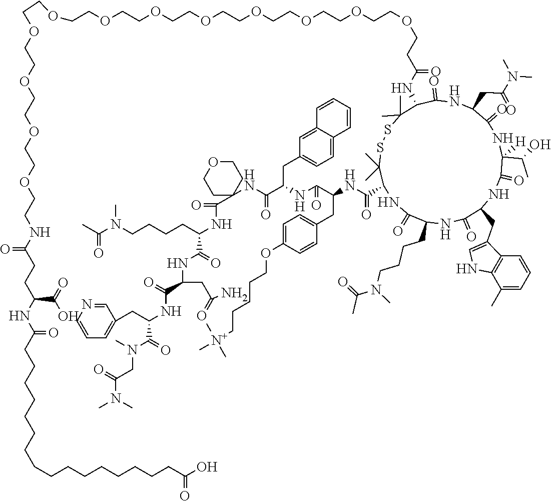 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)CCOCCOCCOCCOCCOCCOCCOCCOCCOCCOCCOCCOCCNC(=O)CC[C@H](NC(=O)CCCCCCCCCCCCCCCCC(=O)O)C(=O)O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] |
| | HOC18gEPEG12CO-Pen(3)-N(N(Me)2)-T-7MeW-K(NMeAc)-Pen(3)-TMAPF-2Nal-THP- |
| | K(NMeAc)-N-3Pya-Sar-CON(Me)2 |
| 96 | 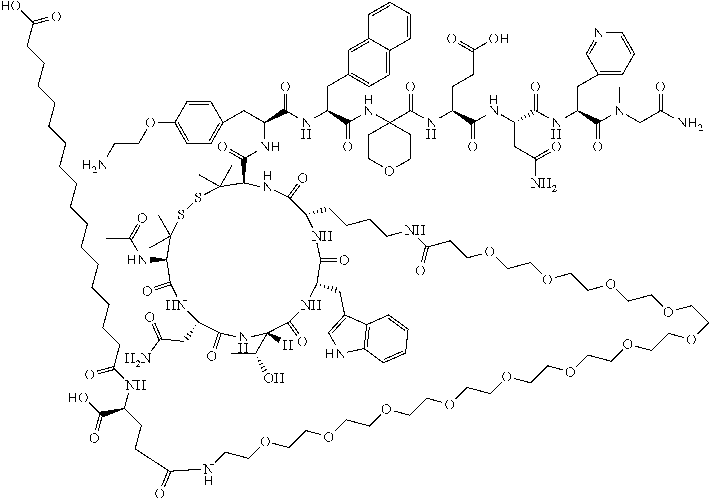 [SMILES: CC(=O)N[C@@H]1C(=O)N[C@@H](CC(N)=O)C(=O)N[C@@H]([C@@H](C)O)C(=O)N[C@@H](Cc2c[nH]c3ccccc23)C(=O)N[C@@H](CCCCNC(=O)CCOCCOCCOCCOCCOCCOCCOCCOCCOCCOCCOCCOCCNC(=O)CC[C@H](NC(=O)CCCCCCCCCCCCCCCCC(=O)O)C(=O)O)C(=O)N[C@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)C(C)(C)SSC1(C)C] |
| | MeCO-Pen(3)-N-T-7MeW-K(PEG12gEC18OH)-Pen(3)-AEF-2Nal-THP-E-N-3Pya-Sar- |
| | CONH2 |
| 97 | 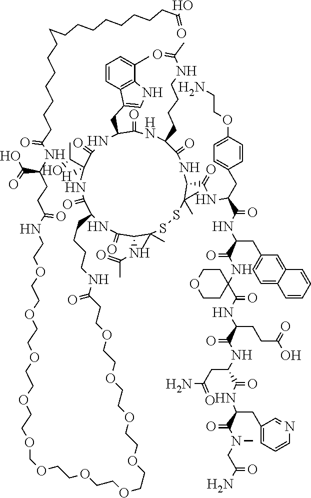 [SMILES: CC(=O)NCCCC[C@@H]1NC(=O)[C@H](Cc2c[nH]c3c(C)cccc23)NC(=O)[C@H]([C@@H](C)O)NC(=O)[C@H](CCCCNC(=O)CCOCCOCCOCCOCCOCCOCCOCOCCOCCOCCOCCOCCNC(=O)CC[C@H](NC(=O)CCCCCCCCCCCCCCCCC(=O)O)C(=O)O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O] |
| | MeCO-Pen(3)-K(PEG12gEC18OH)-T-7MeW-K(Ac)-Pen(3)-AEF-2Nal-THP-E-N-3Pya-Sar- |
| | CONH2 |
| 98 | 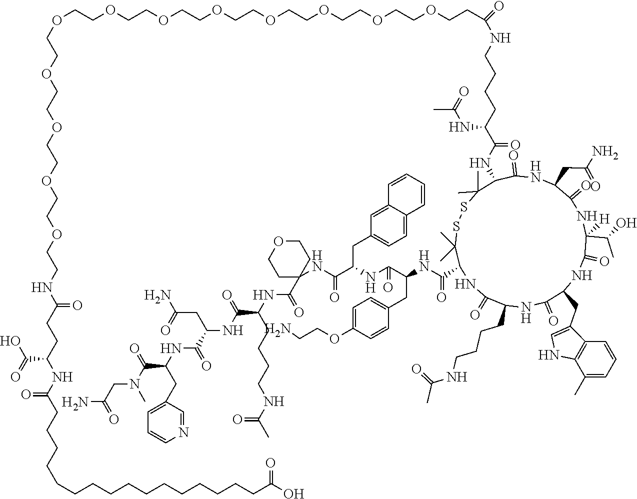 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCCNC(=O)CCOCCOCCOCCOCCOCCOCCOCCOCCOCCOCCOCCOCCNC(=O)CC[C@H](NC(=O)CCCCCCCCCCCCCCCCC(=O)O)C(=O)O)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC1=O] |
| | MeCO-k(PEG12gEC18OH)-Pen(3)-N-T-7MeW-K(Ac)-Pen(3)-AEF-2Nal-THP-K(Ac)-N- |
| | 3Pya-Sar-CONH2 |
| 99 | 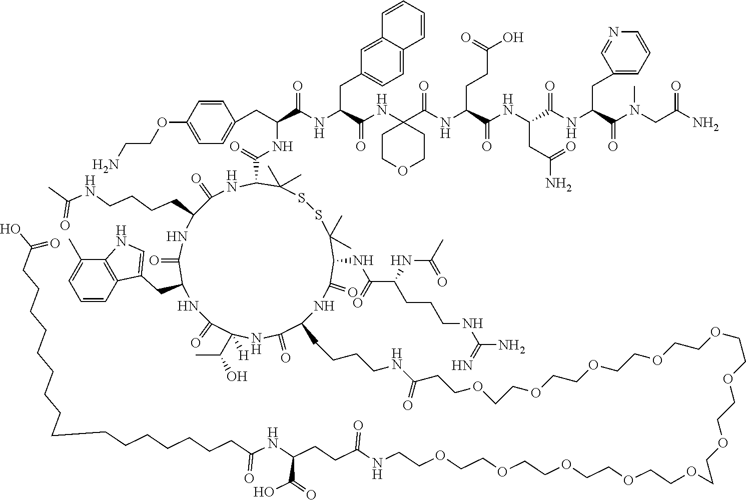 [SMILES: CC(=O)NCCCC[C@@H]1NC(=O)[C@H](Cc2c[nH]c3c(C)cccc23)NC(=O)[C@H]([C@@H](C)O)NC(=O)[C@H](CCCCNC(=O)CCOCCOCCOCCOCCOCCOCCOCCOCCOCCOCCOCCOCCNC(=O)CC[C@H](NC(=O)CCCCCCCCCCCCCCCCC(=O)O)C(=O)O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O] |
| | MeCO-r-Pen(3)-K(PEG12gEC20OH)-T-7MeW-K(Ac)-Pen(3)-AEF-2Nal-THP-E-N-3Pya- |
| | Sar-CONH2 |
| 100 | 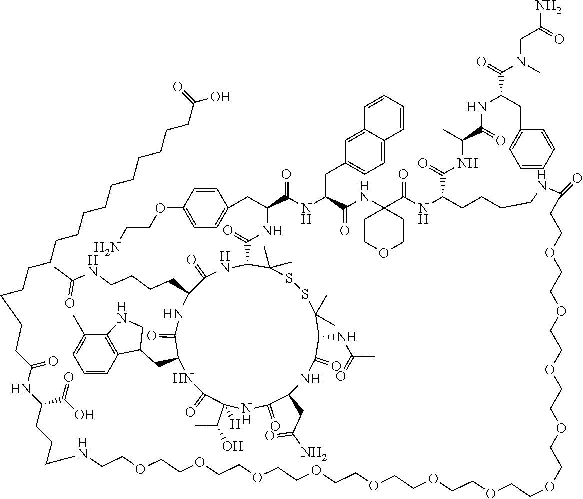 [SMILES: CC(=O)NCCCC[C@@H]1NC(=O)[C@H](CC2CNc3c(C)cccc32)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(=O)CCOCCOCCOCCOCCOCCOCCOCCOCCOCCOCCOCCOCCNCCC[C@H](NC(=O)CCCCCCCCCCCCCCCCC(=O)O)C(=O)O)C(=O)N[C@@H](C)C(=O)N[C@@H](Cc3ccccc3)C(=O)N(C)CC(N)=O)CCOCC2)NC1=O] |
| | MeCO-Pen(3)-N-T-7MeW-K(Ac)-Pen(3)-AEF-2Nal-THP-K(PEG12gEC18OH)-N-3Pya-Sar- |
| | CONH2 |
| 101 | 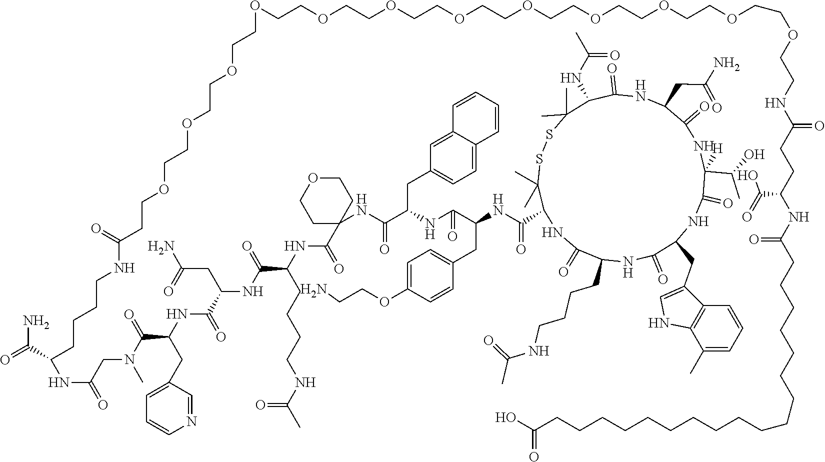 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(=O)N[C@@H](CCCCNC(=O)CCOCCOCCOCCOCCOCCOCCOCCOCCOCCOCCOCCOCCNC(=O)CC[C@H](NC(=O)CCCCCCCCCCCCCCCCCCC(=O)O)C(=O)O)C(N)=O)CCOCC2)NC1=O] |
| | MeCO-Pen(3)-N-T-7MeW-K(Ac)-Pen(3)-AEF-2Nal-THP-K(Ac)-N-3Pya-Sar- |
| | K(PEG12gEC20OH)-CONH2 |
| 102 | 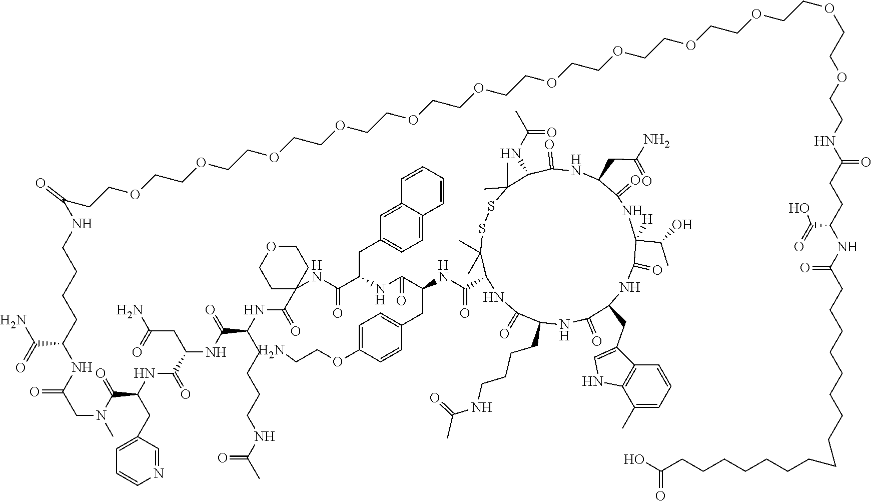 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(=O)N[C@@H](CCCCNC(=O)CCOCCOCCOCCOCCOCCOCCOCCOCCOCCOCCOCCOCCNC(=O)CC[C@H](NC(=O)CCCCCCCCCCCCCCCCC(=O)O)C(=O)O)C(N)=O)CCOCC2)NC1=O] |
| | MeCO-Pen(3)-N-T-7MeW-K(Ac)-Pen(3)-AEF-2Nal-THP-K(Ac)-N-3Pya-Sar- |
| | K(PEG12gEC18OH)-CONH2 |
| 103 | 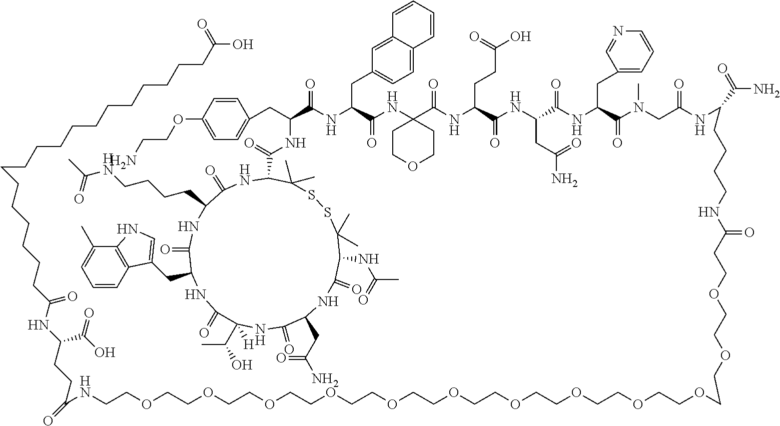 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)CCOCCOCCOCCOCCOCCOCCOCCOCCOCCOCCOCCOCCNC(=O)CC[C@H](NC(=O)CCCCCCCCCCCCCCCCCCC(=O)O)C(=O)O)C(N)=O)CCOCC2)NC1=O] |
| | MeCO-Pen(3)-N-T-7MeW-K(Ac)-Pen(3)-AEF-2Nal-THP-E-N-3Pya-Sar- |
| | K(PEG12gEC20OH)-CONH2 |
| 104 | 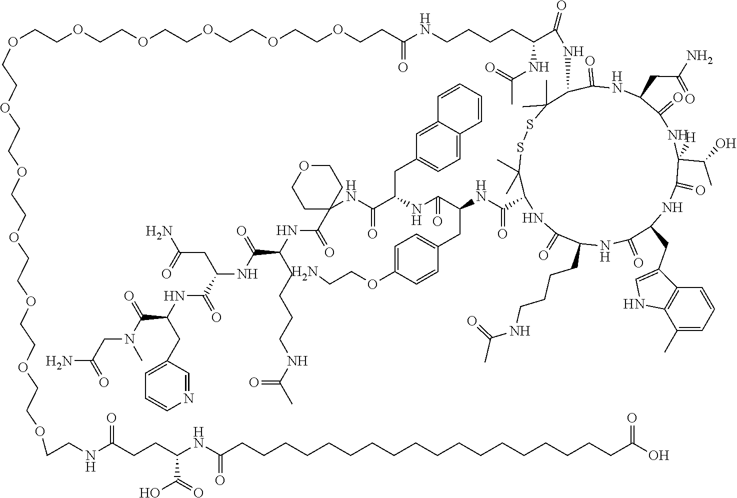 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCCNC(=O)CCOCCOCCOCCOCCOCCOCCOCCOCCOCCOCCOCCOCCNC(=O)CC[C@H](NC(=O)CCCCCCCCCCCCCCCCCCC(=O)O)C(=O)O)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC1=O] |
| | MeCO-k(PEG12gEC20OH)-Pen(3)-N-T-7MeW-K(Ac)-Pen(3)-AEF-2Nal-THP-K(Ac)-N- |
| | 3Pya-Ser-CONH2 |
| 105 | 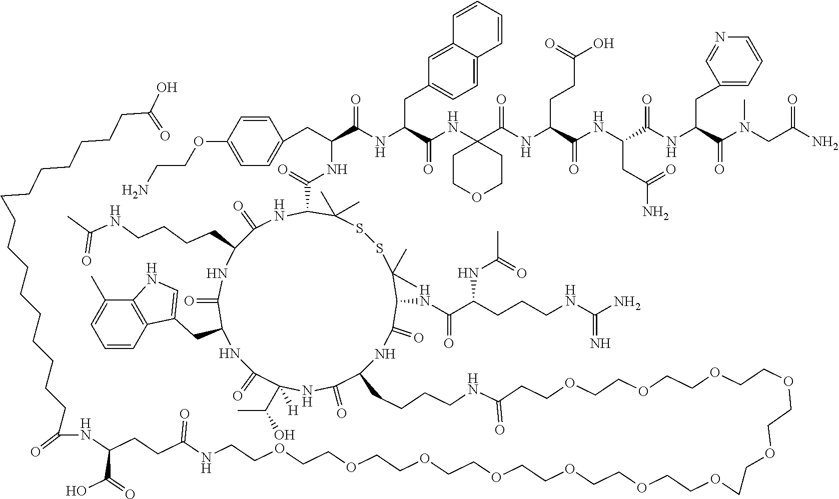 [SMILES: CC(=O)NCCCC[C@@H]1NC(=O)[C@H](Cc2c[nH]c3c(C)cccc23)NC(=O)[C@H]([C@@H](C)O)NC(=O)[C@H](CCCCNC(=O)CCOCCOCCOCCOCCOCCOCCOCCOCCOCCOCCOCCOCCNC(=O)CC[C@H](NC(=O)CCCCCCCCCCCCCCCCC(=O)O)C(=O)O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O] |
| | MeCO-r-Pen(3)-K(PEG12gEC18OH)-T-7MeW-K(Ac)-Pen(3)-AEF-2Nal-THP-E-N-3Pya- |
| | Sar-CONH2 |
| 106 | 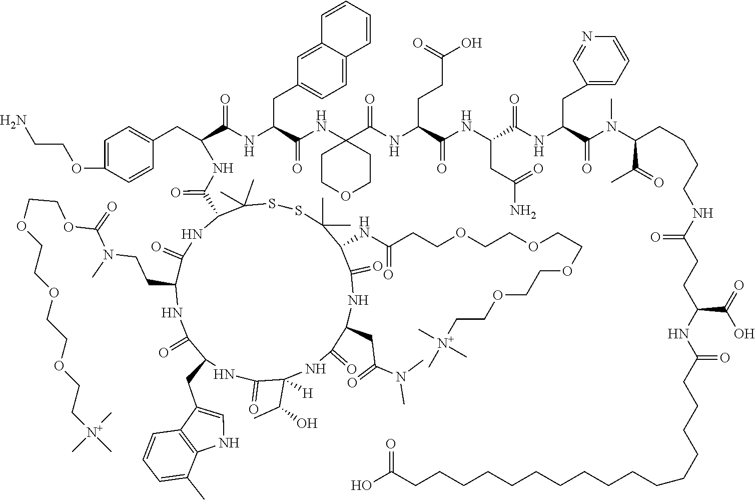 [SMILES: CC(=O)[C@H](CCCCNC(=O)CC[C@H](NC(=O)CCCCCCCCCCCCCCCCC(=O)O)C(=O)O)N(C)C(=O)[C@H](Cc1cccnc1)NC(=O)[C@H](CC(N)=O)NC(=O)[C@H](CCC(=O)O)NC(=O)C1(NC(=O)[C@H](Cc2ccc3ccccc3c2)NC(=O)[C@H](Cc2ccc(OCCN)cc2)NC(=O)[C@H]2NC(=O)[C@H](CCN(C)C(=O)OCCOCCOCCOCC[N+](C)(C)C)NC(=O)[C@H](Cc3c[nH]c4c(C)cccc34)NC(=O)[C@H]([C@@H](C)O)NC(=O)[C@H](CC(=O)N(C)C)NC(=O)[C@@H](NC(=O)CCOCCOCCOCCOCC[N+](C)(C)C)C(C)(C)SSC2(C)C)CCOCC1] |
| | cPEG5aCO-Pen(3)-N(N(Me)2)-T-7MeW-Dab(NMecPEG5a)-Pen(3)-AEF-2Nal-THP-E-N- |
| | 3Pya-NMeK(gEC18OH)-CONH2 |
| 107 | 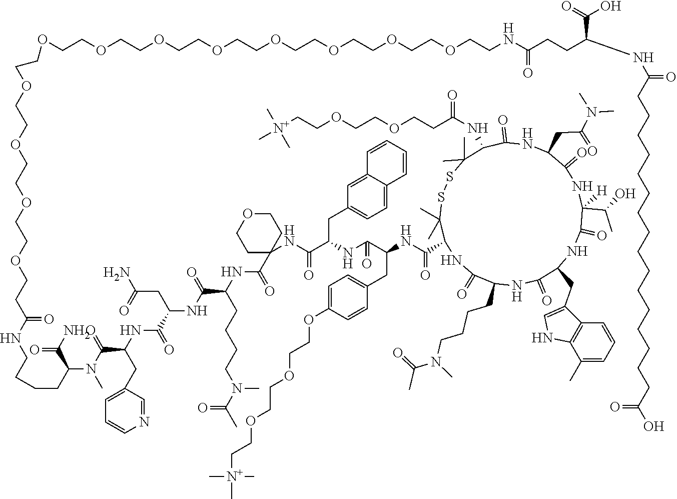 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)CCOCCOCC[N+](C)(C)C)C(C)(C)SSC(C)(C)[C@@H](C(=O)N[C@@H](Cc2ccc(OCCOCCOCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)[C@@H](CCCCNC(=O)CCOCCOCCOCCOCCOCCOCCOCCOCCOCCOCCOCCOCCNC(=O)CC[C@H](NC(=O)CCCCCCCCCCCCCCCCC(=O)O)C(=O)O)C(N)=O)CCOCC2)NC1=O] |
| | cPEG3aCO-Pen(3)-N(N(Me)2)-T-7MeW-K(NMeAc)-Pen(3)-APEG3F-2Nal-THP- |
| | K(NMeAc)-N-3Pya-NMeK(PEG12gEC18OH)-CONH2 |
| 108 | 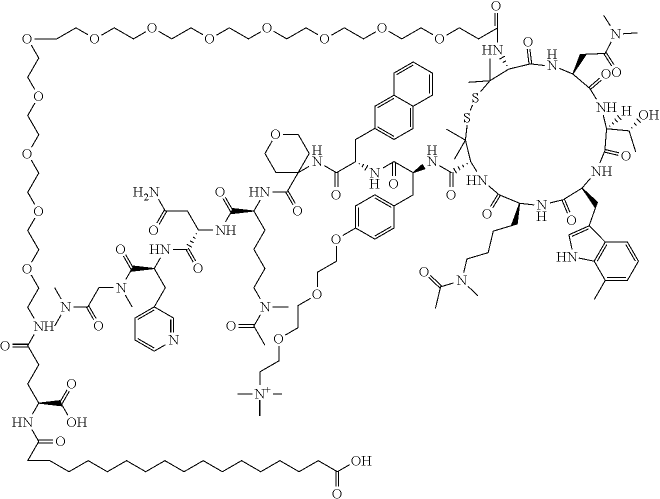 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)CCOCCOCCOCCOCCOCCOCCOCCOCCOCCOCCOCCOCCNC(=O)CC[C@H](NC(=O)CCCCCCCCCCCCCCCCC(=O)O)C(=O)O)C(C)(C)SSC(C)(C)[C@@H](C(=O)N[C@@H](Cc2ccc(OCCOCCOCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] |
| | HOC18gEPEG12CO-Pen(3)-N(N(Me)2)-T-7MeW-K(NMeAc)-Pen(3)-APEG3F-2Nal-THP- |
| | K(NMeAc)-N-3Pya-Sar-CON(Me)2 |
| 109 | 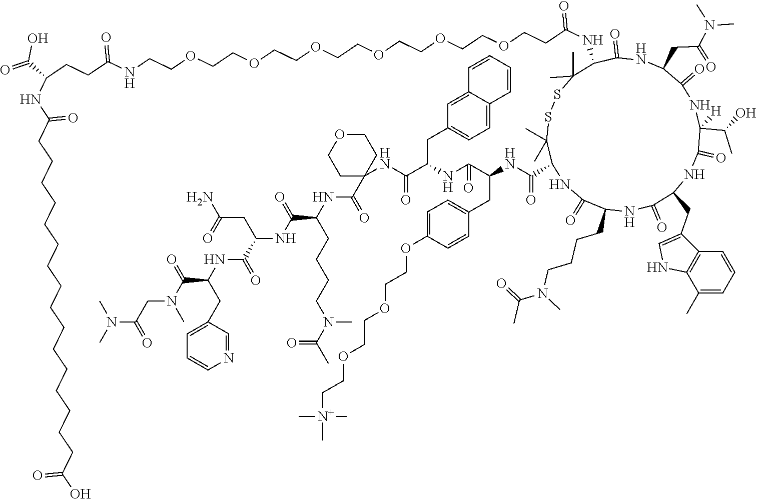 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)CCOCCOCCOCCOCCOCCOCCNC(=O)CC[C@H](NC(=O)CCCCCCCCCCCCCCCCC(=O)O)C(=O)O)C(C)(C)SSC(C)(C)[C@@H](C(=O)N[C@@H](Cc2ccc(OCCOCCOCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] |
| | HOC18gEPEG6CO-Pen(3)-N(N(Me)2)-T-7MeW-K(NMeAc)-Pen(3)-APEG3F-2Nal-THP- |
| | K(NMeAc)-N-3Pya-Sar-CON(Me)2 |
| 110 | 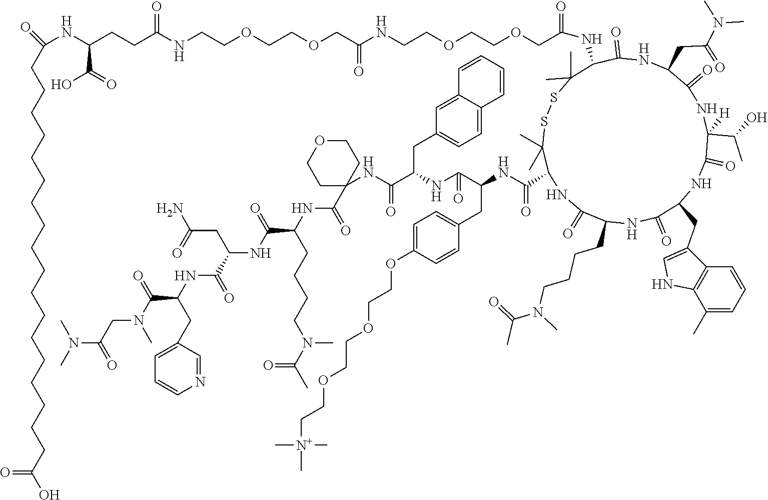 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)COCCOCCNC(=O)COCCOCCNC(=O)CC[C@H](NC(=O)CCCCCCCCCCCCCCCCCCC(=O)O)C(=O)O)C(C)(C)SSC(C)(C)[C@@H](C(=O)N[C@@H](Cc2ccc(OCCOCCOCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] |
| | HOC20gEPEG2PEG2CO-Pen(3)-N(N(Me)2)-T-7MeW-K(NMeAc)-Pen(3)-APEG3F-2Nal- |
| | THP-K(NMeAc)-N-3Pya-Sar-CON(Me)2 |
| 111 | 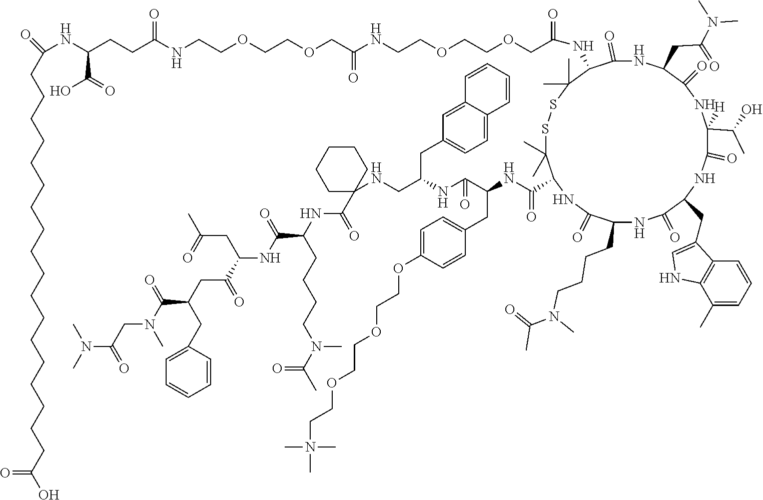 |
| | cPEG5aCO-Pen(3)-N(N(Me)2)-T-7MeW-Dab(NMecPEG5a)-Pen(3)-AEF-2Nal-THP-E-N- |
| | 3Pya-NMeK(PEG12gEC18OH)-CONH2 |
| 112 | 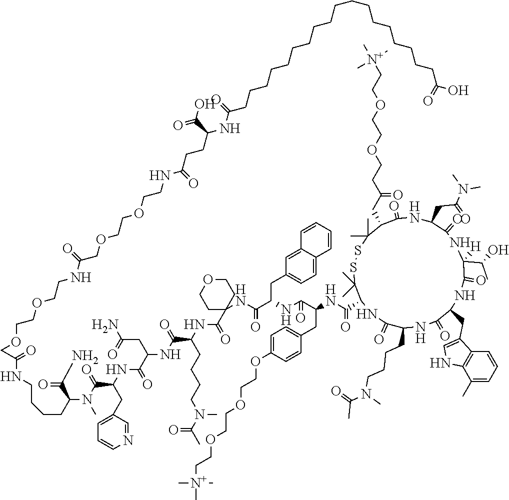 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](CC(=O)CCOCCOCC[N+](C)(C)C)C(C)(C)SSC(C)(C)[C@@H](C(=O)N[C@@H](Cc2ccc(OCCOCCOCC[N+](C)(C)C)cc2)C(=O)N[C@@H](Cc2ccc3ccccc3c2)C(=O)NC2(C(=O)N[C@@H](CCCCN(C)C(C)=O)C(=O)NC(CC(N)=O)C(=O)N[C@@H](Cc3cccnc3)C(=O)N(C)[C@@H](CCCCNC(=O)COCCOCCNC(=O)COCCOCCNC(=O)CC[C@H](NC(=O)CCCCCCCCCCCCCCCCCCC(=O)O)C(=O)O)C(N)=O)CCOCC2)NC1=O] |
| | cPEG3aCO-Pen(3)-N(N(Me)2)-T-7MeW-K(NMeAc)-Pen(3)-APEG3F-2Nal-THP- |
| | K(NMeAc)-N-3Pya-NMeK(PEG2PEG2gEC20OH)-CONH2 |
| 113 | 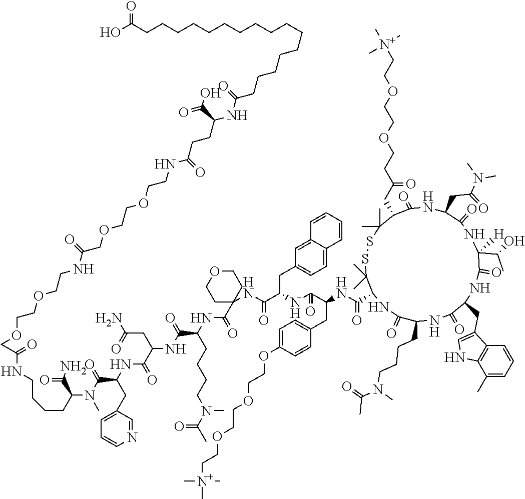 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](CC(=O)CCOCCOCC[N+](C)(C)C)C(C)(C)SSC(C)(C)[C@@H](C(=O)N[C@@H](Cc2ccc(OCCOCCOCC[N+](C)(C)C)cc2)C(=O)N[C@@H](Cc2ccc3ccccc3c2)C(=O)NC2(C(=O)N[C@@H](CCCCN(C)C(C)=O)C(=O)NC(CC(N)=O)C(=O)N[C@@H](Cc3cccnc3)C(=O)N(C)[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)NC1=O] |
| | cPEG3aCO-Pen(3)-N(N(Me)2)-T-7MeW-K(NMeAc)-Pen(3)-APEG3F-2Nal-THP- |
| | K(NMeAc)-N-3Pya-NMeK(PEG2PEG2gEC18OH)-CONH2 |
| 114 | 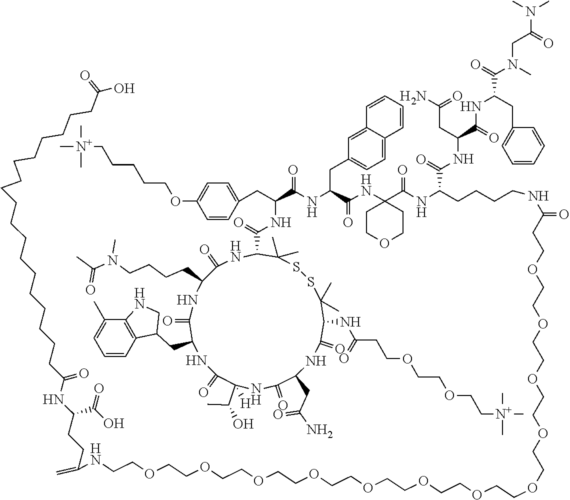 [SMILES: C=C(CC[C@H](NC(=O)CCCCCCCCCCCCCCCCCCC(=O)O)C(=O)O)NCCOCCOCCOCCOCCOCCOCCOCCOCCOCCOCCOCCOCCC(=O)NCCCC[C@H](NC(=O)C1(NC(=O)[C@H](Cc2ccc3ccccc3c2)NC(=O)[C@H](Cc2ccc(OCCCCC[N+](C)(C)C)cc2)NC(=O)[C@H]2NC(=O)[C@H](CCCCN(C)C(C)=O)NC(=O)[C@H](CC3CNc4c(C)cccc43)NC(=O)[C@H]([C@@H](C)O)NC(=O)[C@H](CC(N)=O)NC(=O)[C@@H](NC(=O)CCOCCOCC[N+](C)(C)C)C(C)(C)SSC2(C)C)CCOCC1)C(=O)N[C@@H](CC(N)=O)C(=O)N[C@@H](Cc1ccccc1)C(=O)N(C)CC(=O)N(C)C] |
| | cPEG3aCO-Pen(3)-N(N(Me)2)-T-7MeW-K(NMeAc)-Pen(3)-TMAPF-2Nal-THP- |
| | K(PEG12gEC20OH)-N-3Pya-Sar-CON(Me)2 |
| 115 | 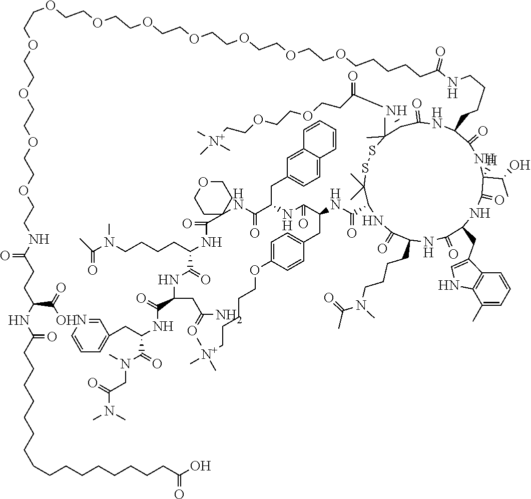 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CCCCNC(=O)CCCCCOCCOCCOCCOCCOCCOCCOCCOCCOCCOCCOCCNC(=O)CC[C@H](NC(=O)CCCCCCCCCCCCCCCCC(=O)O)C(=O)O)NC(=O)[C@@H](NC(=O)CCOCCOCC[N+](C)(C)C)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] |
| | cPEG3aCO-Pen(3)-K(PEG12gEC20OH)-T-7MeW-K(NMeAc)-Pen(3)-TMAPF-2Nal-THP- |
| | K(NMeAc)-N-3Pya-Sar-CON(Me)2 |
| 116 | 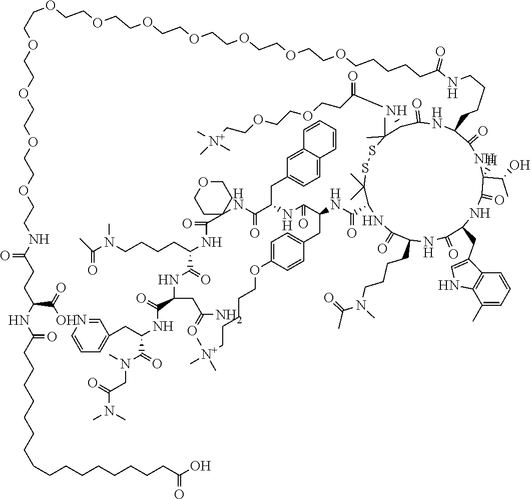 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CCCCNC(=O)CCCCCOCCOCCOCCOCCOCCOCCOCCOCCOCCOCCOCCNC(=O)CC[C@H](NC(=O)CCCCCCCCCCCCCCCCC(=O)O)C(=O)O)NC(=O)[C@@H](NC(=O)CCOCCOCC[N+](C)(C)C)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] |
| | HOC18gEPEG12CO-Pen(3)-N-T-K(Ac)-Pen(3)-AEF-2Nal-THP-E-N-3Pya-Sar-CONH2 |
| 117 | 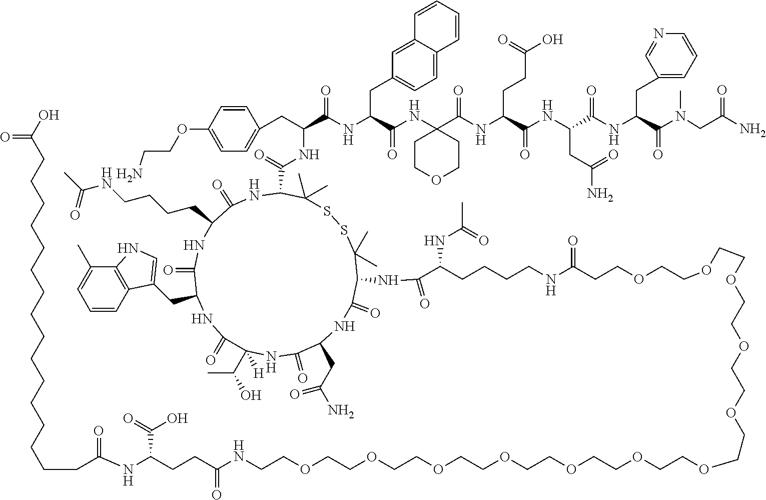 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCCNC(=O)CCOCCOCCOCCOCCOCCOCCOCCOCCOCCOCCOCCOCCNC(=O)CC[C@H](NC(=O)CCCCCCCCCCCCCCCCC(=O)O)C(=O)O)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O] |
| | MeCO-k(PEG12gEC18OH)-Pen(3)-N-T-7MeW-K(Ac)-Pen(3)-AEF-2Nal-THP-E-N-3Pya- |
| | Sar-CONH2 |
| 118 | 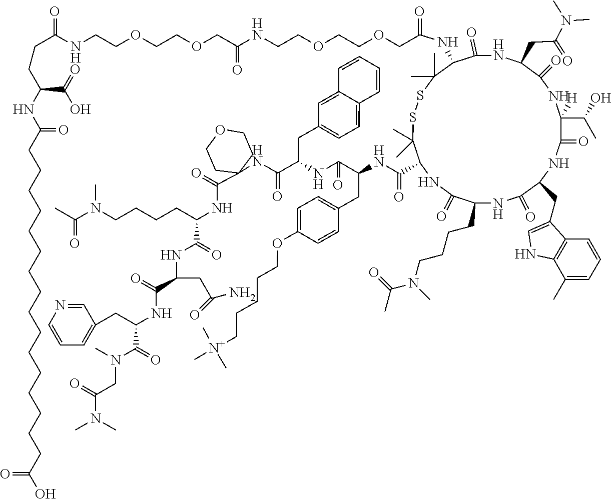 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)COCCOCCNC(=O)COCCOCCNC(=O)CC[C@H](NC(=O)CCCCCCCCCCCCCCCCC(=O)O)C(=O)O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] |
| | HOC18gEPEG2PEG2CO-Pen(3)-N(N(Me)2)-T-7MeW-K(NMeAc)-Pen(3)-TMAPF-2Nal- |
| | THP-K(NMeAc)-N-3Pya-Sar-CON(Me)2 |
| 119 | 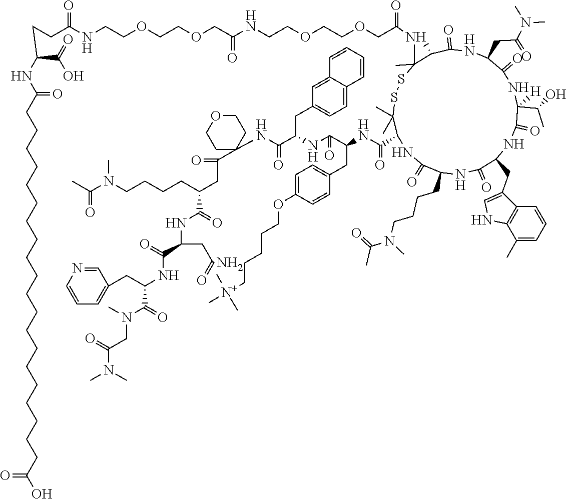 [SMILES: CC(=O)N(C)CCCC[C@H](CC(=O)C1(NC(=O)[C@H](Cc2ccc3ccccc3c2)NC(=O)[C@H](Cc2ccc(OCCCCC[N+](C)(C)C)cc2)NC(=O)[C@H]2NC(=O)[C@H](CCCCN(C)C(C)=O)NC(=O)[C@H](Cc3c[nH]c4c(C)cccc34)NC(=O)[C@H]([C@@H](C)O)NC(=O)[C@H](CC(=O)N(C)C)NC(=O)[C@@H](NC(=O)COCCOCCNC(=O)COCCOCCNC(=O)CC[C@H](NC(=O)CCCCCCCCCCCCCCCCCCCCC(=O)O)C(=O)O)C(C)(C)SSC2(C)C)CCOCC1)C(=O)N[C@@H](CC(N)=O)C(=O)N[C@@H](Cc1cccnc1)C(=O)N(C)CC(=O)N(C)C] |
| | HOC20gEPEG2PEG2CO-Pen(3)-N(N(Me)2)-T-7MeW-K(NMeAc)-Pen(3)-TMAPF-2Nal- |
| | THP-K(NMeAc)-N-3Pya-Sar-CON(Me)2 |
| 120 | 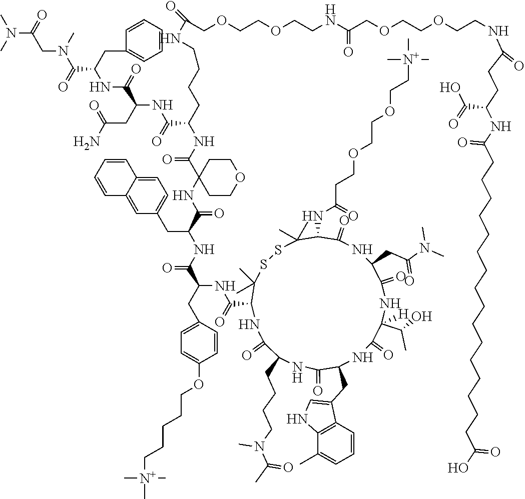 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)CCOCCOCC[N+](C)(C)C)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(=O)N[C@@H](CC(N)=O)C(=O)N[C@@H](Cc3ccccc3)C(=O)N(C)CC(=O)N(C)C)CCOCC2)NC1=O] |
| | cPEG3aCO-Pen(3)-N(N(Me)2)-T-7MeW-K(NMeAc)-Pen(3)-TMAPF-2Nal-THP- |
| | K(PEG2PEG2gEC18OH)-N-3Pya-Sar-CON(Me)2 |
| 121 | 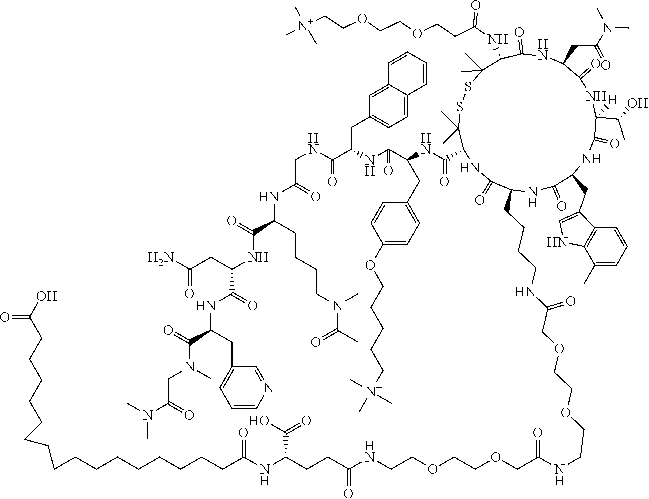 [SMILES: CC(=O)N(C)CCCC[C@H](NC(=O)CNC(=O)[C@H](Cc1ccc2ccccc2c1)NC(=O)[C@H](Cc1ccc(OCCCCC[N+](C)(C)C)cc1)NC(=O)[C@H]1NC(=O)[C@H](CCCCNC(=O)COCCOCCNC(=O)COCCOCCNC(=O)CC[C@H](NC(=O)CCCCCCCCCCCCCCCCC(=O)O)C(=O)O)NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)CCOCCOCC[N+](C)(C)C)C(C)(C)SSC1(C)C)C(=O)N[C@@H](CC(N)=O)C(=O)N[C@@H](Cc1cccnc1)C(=O)N(C)CC(=O)N(C)C] |
| | cPEG3aCO-Pen(3)-N(N(Me)2)-T-7MeW-K(PEG2PEG2gEC18OH)-Pen(3)-TMAPF-2Nal- |
| | THP-K(NMeAc)-N-3Pya-Sar-CON(Me)2 |
| 122 | 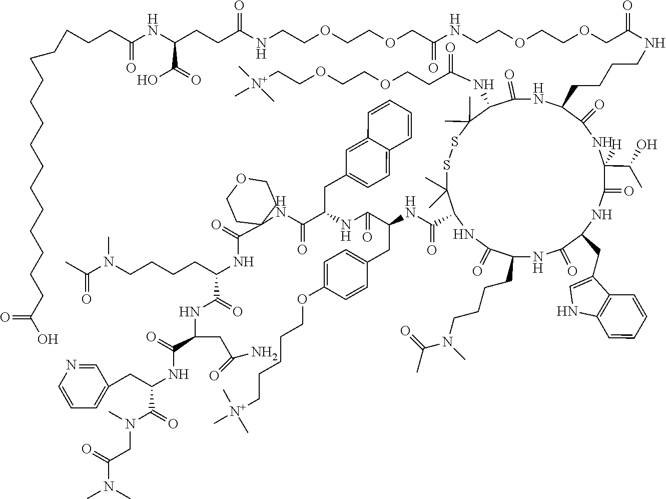 [SMILES: CC(=O)N(C)CCCC[C@@H]1NC(=O)[C@H](Cc2c[nH]c3ccccc23)NC(=O)[C@H]([C@@H](C)O)NC(=O)[C@H](CCCCNC(=O)COCCOCCNC(=O)COCCOCCNC(=O)CC[C@H](NC(=O)CCCCCCCCCCCCCCCCC(=O)O)C(=O)O)NC(=O)[C@@H](NC(=O)CCOCCOCC[N+](C)(C)C)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] |
| | cPEG3aCO-Pen(3)-K(PEG2PEG2gEC18OH)-T-7MeW-K(NMeAc)-Pen(3)-TMAPF-2Nal- |
| | THF-K(NMeAc)-N-3Pya-Sar-CON(Me)2 |
| 123 | 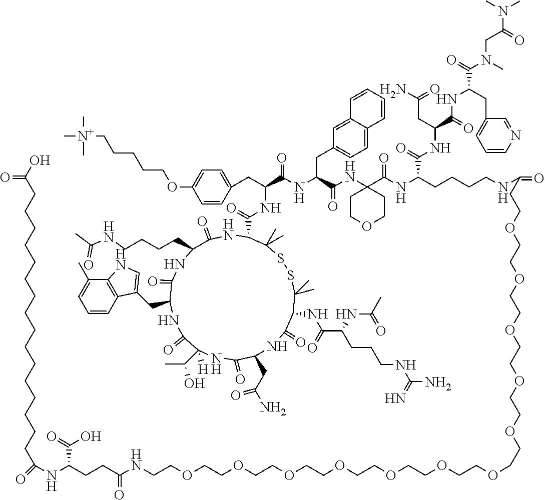 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(=O)CCOCCOCCOCCOCCOCCOCCOCCOCCOCCOCCOCCOCCNC(=O)CC[C@H](NC(=O)CCCCCCCCCCCCCCCCC(=O)O)C(=O)O)C(=O)N[C@@H](CC(N)=O)C(=O)N[C@@H](Cc3cccnc3)C(=O)N(C)CC(=O)N(C)C)CCOCC2)NC1=O] |
| | cPEG3aCO-Pen(3)-N(N(Me)2)-T-7MeW-K(NMeAc)-Pen(3)-TMAPF-2Nal-THP- |
| | K(PEG12gEC18OH)-N-3Pya-Sar-CON(Me)2 |
| 124 | 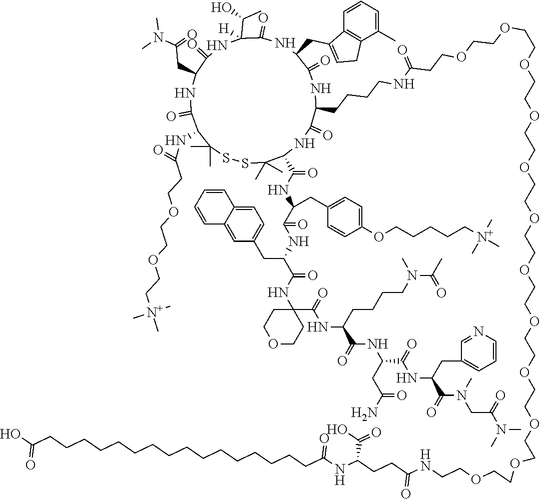 [SMILES: CC(=O)N(C)CCCC[C@H](NC(=O)C1(NC(=O)[C@H](Cc2ccc3ccccc3c2)NC(=O)[C@H](Cc2ccc(OCCCCC[N+](C)(C)C)cc2)NC(=O)[C@H]2NC(=O)[C@H](CCCCNC(=O)CCOCCOCCOCCOCCOCCOCCOCCOCCOCCOCCOCCOCCNC(=O)CC[C@H](NC(=O)CCCCCCCCCCCCCCCCC(=O)O)C(=O)O)NC(=O)[C@H](CC3=CCc4c(C)cccc43)NC(=O)[C@H]([C@@H](C)O)NC(=O)[C@H](CC(=O)N(C)C)NC(=O)[C@@H](NC(=O)CCOCCOCC[N+](C)(C)C)C(C)(C)SSC2(C)C)CCOCC1)C(=O)N[C@@H](CC(N)=O)C(=O)N[C@@H](Cc1cccnc1)C(=O)N(C)CC(=O)N(C)C] |
| | cPEG3aCO-Pen(3)-N(N(Me)2)-T-7MeW-K(PEG12gEC18OH)-Pen(3)-TMAPF-2Nal-THP- |
| | K(NMeAc)-N-3Pya-Sar-CON(Me)2 |
| 125 | 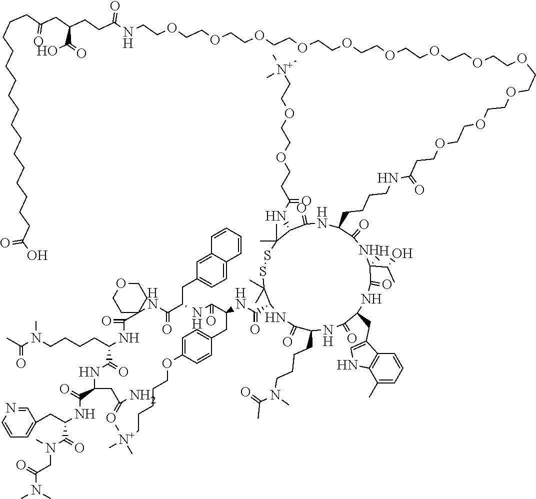 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CCCCNC(=O)CCOCCOCCOCCOCCOCCOCCOCCOCCOCCOCCOCCOCCNC(=O)CC[C@H](CC(=O)CCCCCCCCCCCCCCCCC(=O)O)C(=O)O)NC(=O)[C@@H](NC(=O)CCOCCOCC[N+](C)(C)C)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] |
| | cPEG3aCO-Pen(3)-K(PEG12gEC18OH)-T-7MeW-K(NMeAc)-Pen(3)-TMAPF-2Nal-THP- |
| | K(NMeAc)-N-3Pya-Sar-CON(Me)2 |
| 126 | 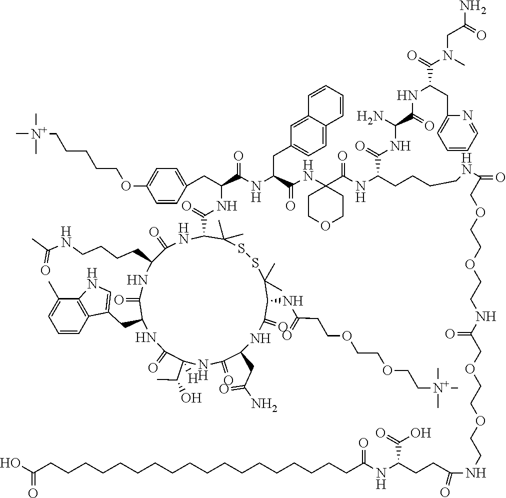 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)CCOCCOCC[N+](C)(C)C)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(=O)COCCOCCNC(=O)COCCOCCNC(=O)CC[C@H](NC(=O)CCCCCCCCCCCCCCCCCCC(=O)O)C(=O)O)C(=O)N[C@@H](N)C(=O)N[C@@H](Cc3ccccn3)C(=O)N(C)CC(N)=O)CCOCC2)NC1=O] |
| | cPEG3aCO-Pen(3)-N(N(Me)2)-T-7MeW-K(NMeAc)-Pen(3)-TMAPF-2Nal-THP- |
| | K(PEG2PEG2gEC20OH)-N-3Pya-Sar-CON(Me)2 |
| 127 | 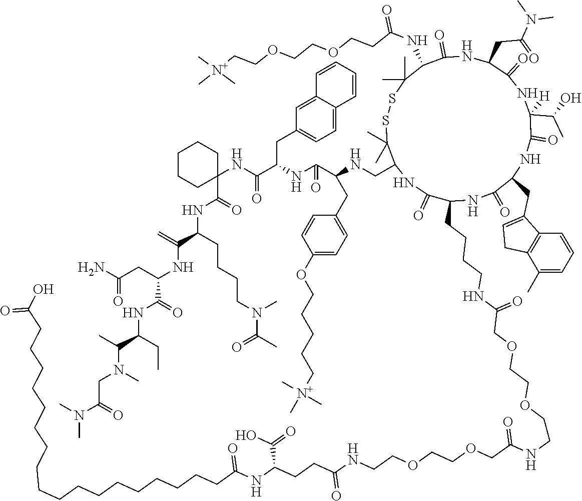 [SMILES: C=C(N[C@@H](CC(N)=O)C(=O)N[C@@H](CC)C(C)N(C)CC(=O)N(C)C)[C@H](CCCCN(C)C(C)=O)NC(=O)C1(NC(=O)[C@H](Cc2ccc3ccccc3c2)NC(=O)[C@H](Cc2ccc(OCCCCC[N+](C)(C)C)cc2)NCC2NC(=O)[C@H](CCCCNC(=O)COCCOCCNC(=O)COCCOCCNC(=O)CC[C@H](NC(=O)CCCCCCCCCCCCCCCCCCC(=O)O)C(=O)O)NC(=O)[C@H](CC3=CCc4c(C)cccc43)NC(=O)[C@H]([C@@H](C)O)NC(=O)[C@H](CC(=O)N(C)C)NC(=O)[C@@H](NC(=O)CCOCCOCC[N+](C)(C)C)C(C)(C)SSC2(C)C)CCCCC1] |
| | cPEG3aCO-Pen(3)-N(N(Me)2)-T-7MeW-K(PEG2PEG2gEC20OH)-Pen(3)-TMAPF-2Nal- |
| | THP-K(NMeAc)-N-3Pya-Sar-CON(Me)2 |
| 128 | 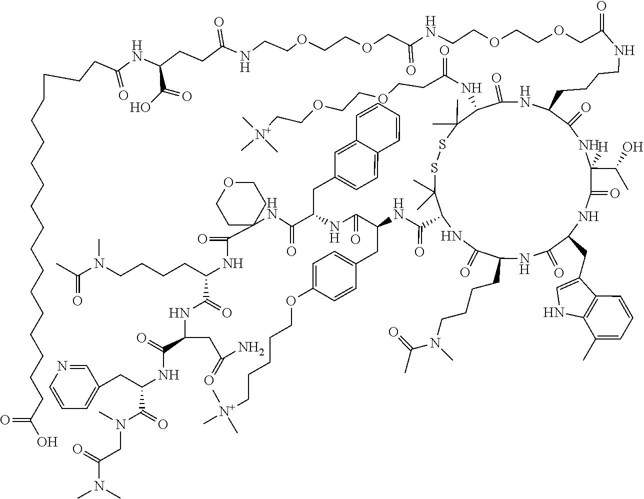 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CCCCNC(=O)COCCOCCNC(=O)COCCOCCNC(=O)CC[C@H](NC(=O)CCCCCCCCCCCCCCCCCCC(=O)O)C(=O)O)NC(=O)[C@@H](NC(=O)CCOCCOCC[N+](C)(C)C)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] |
| | cPEG3aCO-Pen(3)-K(PEG2PEG2gEC20OH)-T-7MeW-K(NMeAc)-Pen(3)-TMAPF-2Nal- |
| | THP-K(NMeAc)-N-3Pya-Sar-CON(Me)2 |
| 129 | 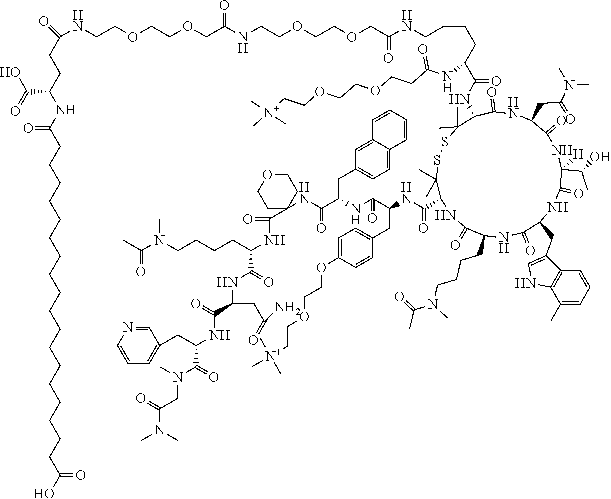 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)[C@@H](CCCCNC(=O)COCCOCCNC(=O)COCCOCCNC(=O)CC[C@H](NC(=O)CCCCCCCCCCCCCCCCCCC(=O)O)C(=O)O)NC(=O)CCOCCOCC[N+](C)(C)C)C(C)(C)SSC(C)(C)[C@@H](C(=O)N[C@@H](Cc2ccc(OCCOCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] |
| | cPEG3aCO-K(PEG2PEG2gEC20OH)-Pen(3)-N(N(Me)2)-T-7MeW-K(NMeAc)-Pen(3)- |
| | TMAPF-2Nal-THP-K(NMeAc)-N-3Pya-Sar-CON(Me)2 |
| 130 | 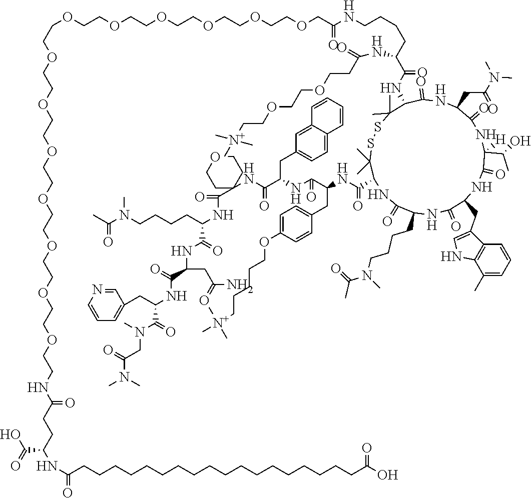 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)[C@@H](CCCCNC(=O)COCCOCCOCCOCCOCCOCCOCCOCCOCCOCCOCCOCCOCCNC(=O)CC[C@H](NC(=O)CCCCCCCCCCCCCCCCCCC(=O)O)C(=O)O)NC(=O)CCOCCOCC[N+](C)(C)C)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] |
| | cPEG3aCO-k(PEG12gEC20OH)-Pen(3)-N(N(Me)2)-T-7MeW-K(NMeAc)-Pen(3)-TMAPF- |
| | 2Nal-THP-K(NMeAc)-N-3Pya-Sar-CON(Me)2 |
| 131 | 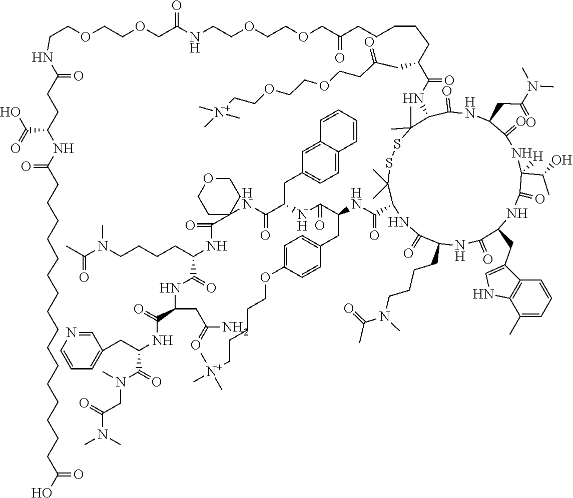 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)[C@@H](CCCCCC(=O)COCCOCCNC(=O)COCCOCCNC(=O)CC[C@H](NC(=O)CCCCCCCCCCCCCCCCC(=O)O)C(=O)O)CC(=O)CCOCCOCC[N+](C)(C)C)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] |
| | cPEG3aCO-k(PEG2PEG2gEC18OH)-Pen(3)-N(N(Me)2)-T-7MeW-K(NMeAc)-Pen(3)- |
| | TMAPF-2Nal-THP-K(NMeAc)-N-3Pya-Sar-CON(Me)2 |
| 132 | 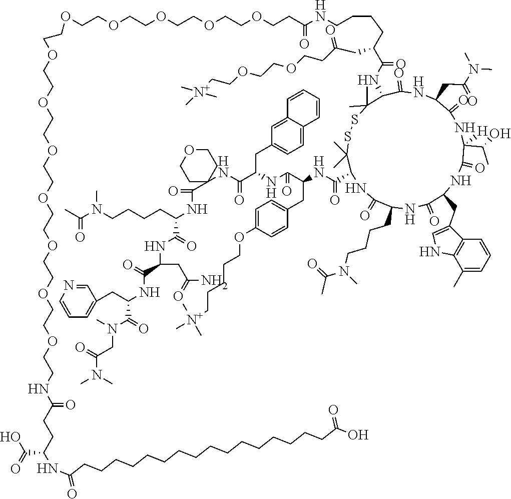 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)[C@@H](CCCCNC(=O)CCOCCOCCOCCOCCOCCOCCOCCOCCOCCOCCOCCOCCNC(=O)CC[C@H](NC(=O)CCCCCCCCCCCCCCCCC(=O)O)C(=O)O)CC(=O)CCOCCOCC[N+](C)(C)C)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] |
| | cPEG3aCO-k(PEG12gEC18OH)-Pen(3)-N(N(Me)2)-T-7MeW-K(NMeAc)-Pen(3)-TMAPF- |
| | 2Nal-THP-K(NMeAc)-N-3Pya-Sar-CON(Me)2 |
| 133 | 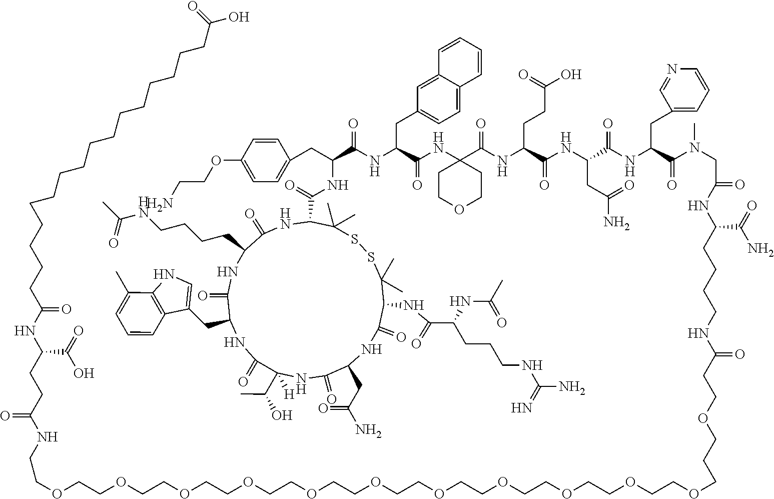 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)CCOCCCOCCOCCOCCOCCOCCOCCOCCOCCOCCOCCOCCNC(=O)CC[C@H](NC(=O)CCCCCCCCCCCCCCCCC(=O)O)C(=O)O)C(N)=O)CCOCC2)NC1=O] |
| | MeCO-r-Pen(3)-N-T-7MeW-K(Ac)-Pen(3)-AEF-2Nal-THP-E-N-3Pya-Sar- |
| | K(PEG12gEC18OH)-CONH2 |
| 134 | 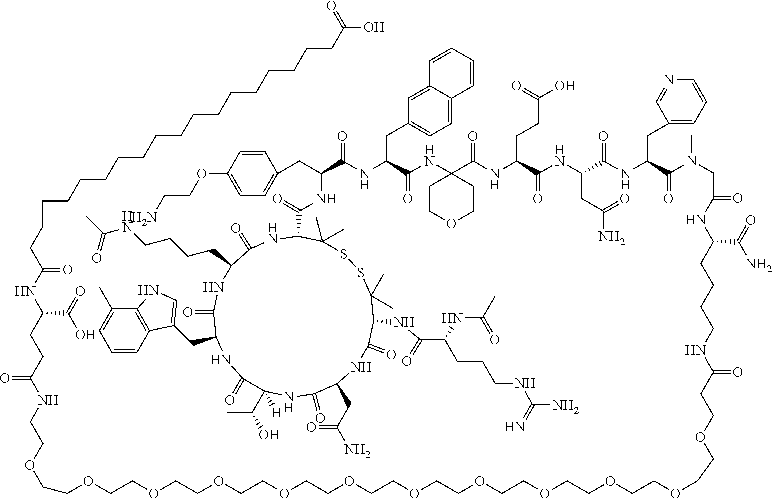 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)CCOCCOCCOCCOCCOCCOCCOCCOCCOCCOCCOCCOCCNC(=O)CC[C@H](NC(=O)CCCCCCCCCCCCCCCCCCC(=O)O)C(=O)O)C(N)=O)CCOCC2)NC1=O] |
| | MeCO-r-Pen(3)-N-T-7MeW-K(Ac)-Pen(3)-AEF-2Nal-THP-E-N-3Pya-Sar- |
| | K(PEG12gEC20OH)-CONH2 |
| 135 | 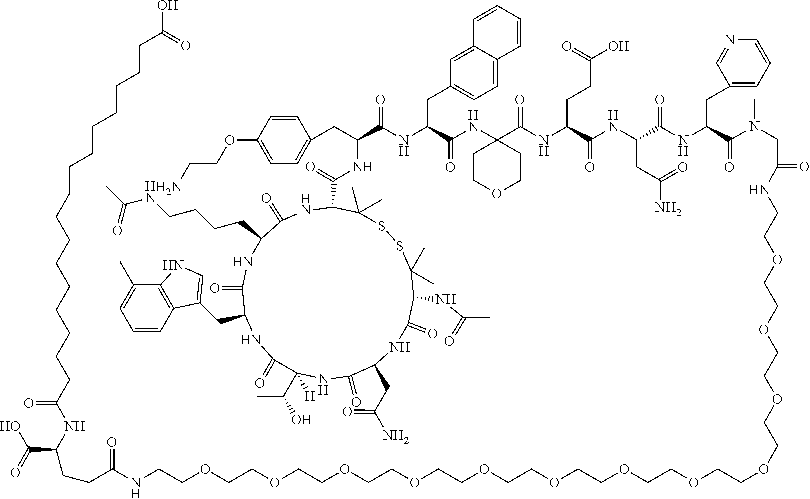 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)NCCOCCOCCOCCOCCOCCOCCOCCOCCOCCOCCOCCOCCNC(=O)CC[C@H](NC(=O)CCCCCCCCCCCCCCCCC(=O)O)C(=O)O)CCOCC2)NC1=O] |
| | MeCO-Pen(3)-N-T-7MeW-K(Ac)-Pen(3)-AEF-2Nal-THP-E-N-3Pya-Sar- |
| | CO(PEG12gEC18OH) |
| 136 | 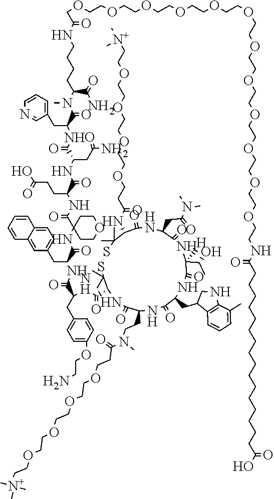 [SMILES: Cc1cccc2c1NCC2C[C@@H]1NC(=O)[C@H]([C@@H](C)O)NC(=O)[C@H](CC(=O)N(C)C)NC(=O)[C@@H](NC(=O)CCOCCOCCOCCOCC[N+](C)(C)C)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)[C@@H](CCCCNC(=O)COCCOCCOCCOCCOCCOCCOCCOCCOCCOCCOCCOCCNC(=O)CCCCCCCCCCCCCCCCC(=O)O)C(N)=O)CCOCC2)NC(=O)[C@H](CCN(C)C(=O)CCOCCOCCOCCOCC[N+](C)(C)C)NC1=O] |
| | cPEG5aCO-Pen(3)-N(N(Me)2)-T-7MeW-Dab(NMecPEG5a)-Pen(3)-AEF-2Nal-THP-E-N- |
| | 3Pya-NMeK(PEG12C18OH)-CONH2 |
| 137 | 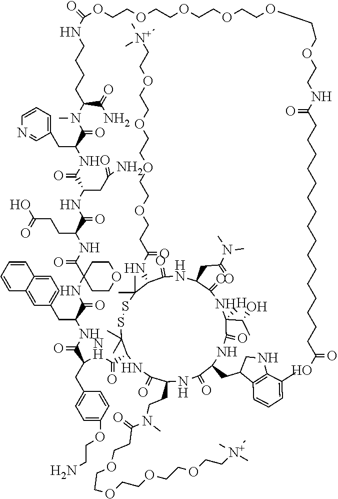 [SMILES: Cc1cccc2c1NCC2C[C@@H]1NC(=O)[C@H]([C@@H](C)O)NC(=O)[C@H](CC(=O)N(C)C)NC(=O)[C@@H](NC(=O)CCOCCOCCOCCOCC[N+](C)(C)C)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)[C@@H](CCCCNC(=O)OCCOCCOCCOCCOCCOCCNC(=O)CCCCCCCCCCCCCCCCC(=O)O)C(N)=O)CCOCC2)NC(=O)[C@H](CCN(C)C(=O)CCOCCOCCOCCOCC[N+](C)(C)C)NC1=O] |
| | cPEG5aCO-Pen(3)-N(N(Me)2)-T-7MeW-Dab(NMecPEG5a)-Pen(3)-AEF-2Nal-THP-E-N- |
| | 3Pya-NMeK(PEG6C18OH)-CONH2 |
| 138 | 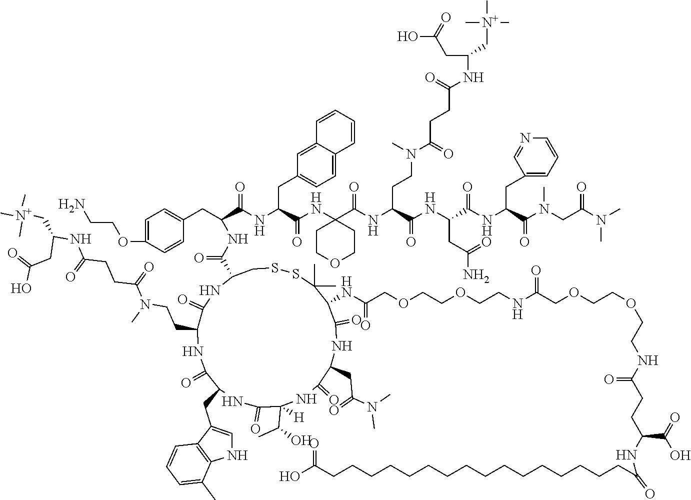 [SMILES: Cc1cccc2c(C[C@@H]3NC(=O)[C@H]([C@@H](C)O)NC(=O)[C@H](CC(=O)N(C)C)NC(=O)[C@@H](NC(=O)COCCOCCNC(=O)COCCOCCNC(=O)CC[C@H](NC(=O)CCCCCCCCCCCCCCCCC(=O)O)C(=O)O)C(C)(C)SSC[C@@H](C(=O)N[C@@H](Cc4ccc(OCCN)cc4)C(=O)N[C@@H](Cc4ccc5ccccc5c4)C(=O)NC4(C(=O)N[C@@H](CCN(C)C(=O)CCC(=O)N[C@H](CC(=O)O)C[N+](C)(C)C)C(=O)N[C@@H](CC(N)=O)C(=O)N[C@@H](Cc5cccnc5)C(=O)N(C)CC(=O)N(C)C)CCOCC4)NC(=O)[C@H](CCN(C)C(=O)CCC(=O)N[C@H](CC(=O)O)C[N+](C)(C)C)NC3=O)c[nH]c12] |
| | HOC18gEPEG2PEG2CO-Pen(3)-N(N(Me)2)-T-7MeW-Dab(NMecarn)-Pen(3)-AEF-2Nal- |
| | THP-Dab(NMecarn)-N-3Pya-Sar-CON(Me)2 |
| 139 | 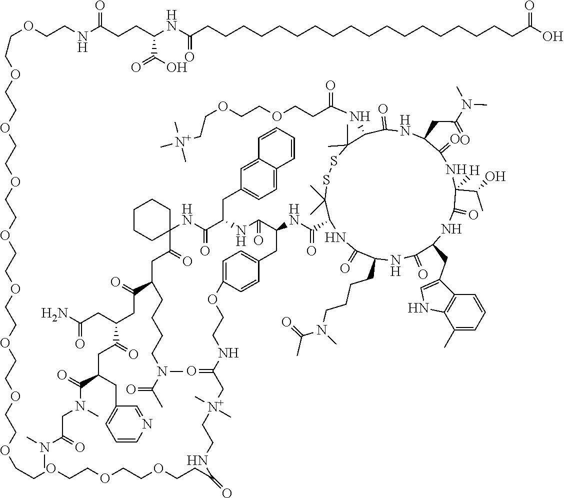 [SMILES: CC(=O)N(C)CCCC[C@H](CC(=O)C1(NC(=O)[C@H](Cc2ccc3ccccc3c2)NC(=O)[C@H](Cc2ccc(OCCNC(=O)C[N+](C)(C)CCNC(=O)CCOCCOCCOCCOCCOCCOCCOCCOCCOCCOCCOCCOCCNC(=O)CC[C@H](NC(=O)CCCCCCCCCCCCCCCCCCC(=O)O)C(=O)O)cc2)NC(=O)[C@H]2NC(=O)[C@H](CCCCN(C)C(C)=O)NC(=O)[C@H](Cc3c[nH]c4c(C)cccc34)NC(=O)[C@H]([C@@H](C)O)NC(=O)[C@H](CC(=O)N(C)C)NC(=O)[C@@H](NC(=O)CCOCCOCC[N+](C)(C)C)C(C)(C)SSC2(C)C)CCCCC1)C(=O)C[C@@H](CC(N)=O)C(=O)C[C@@H](Cc1cccnc1)C(=O)N(C)CC(=O)N(C)C] |
| | cPEG3aCO-Pen(3)-N(N(Me)2)-T-7MeW-K(NMeAc)-Pen(3)-AEF(SP6PEG12gEC20OH)- |
| | 2Nal-THP-K(NMeAc)-N-3Pya-Sar-CON(Me)2 |
| 140 | 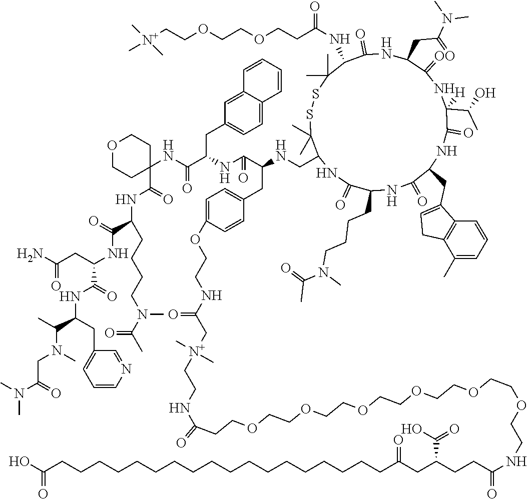 [SMILES: CC(=O)N(C)CCCC[C@@H]1NC(=O)[C@H](CC2=CCc3c(C)cccc32)NC(=O)[C@H]([C@@H](C)O)NC(=O)[C@H](CC(=O)N(C)C)NC(=O)[C@@H](NC(=O)CCOCCOCC[N+](C)(C)C)C(C)(C)SSC(C)(C)C(CN[C@@H](Cc2ccc(OCCNC(=O)C[N+](C)(C)CCNC(=O)CCOCCOCCOCCOCCOCCOCCNC(=O)CC[C@H](CC(=O)CCCCCCCCCCCCCCCCCCC(=O)O)C(=O)O)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(C)N(C)CC(=O)N(C)C)CCOCC2)NC1=O] |
| | cPEG3aCO-Pen(3)-N(N(Me)2)-T-7MeW-K(NMeAc)-Pen(3)-AEF(SP6PEG6gEC20OH)- |
| | 2Nal-THP-K(NMeAc)-N-3Pya-Sar-CON(Me)2 |
| 141 | 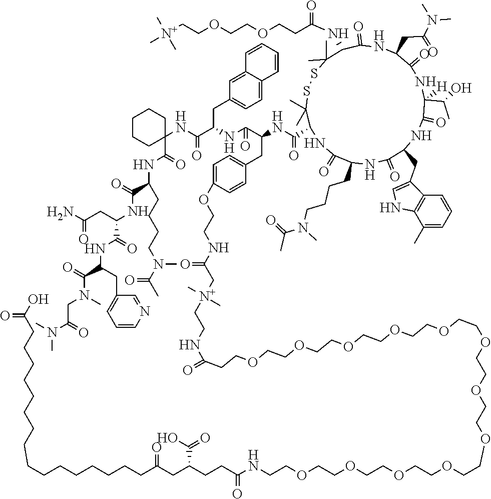 |
| | cPEG3aCO-Pen(3)-N(N(Me)2)-T-7MeW-K(NMeAc)-Pen(3)-AEF(SP6PEG12gEC18OH)- |
| | 2Nal-THP-K(NMeAc)-N-3Pya-Sar-CON(Me)2 |
| 142 | 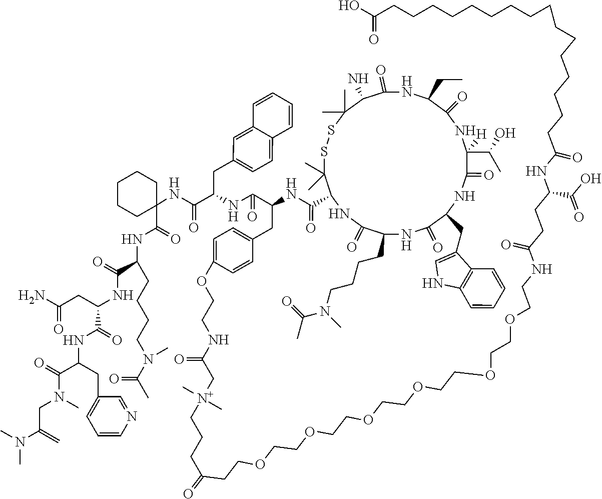 [SMILES: C=C(CN(C)C(=O)C(Cc1cccnc1)NC(=O)[C@H](CC(N)=O)NC(=O)[C@H](CCCCN(C)C(C)=O)NC(=O)C1(NC(=O)[C@H](Cc2ccc3ccccc3c2)NC(=O)[C@H](Cc2ccc(OCCNC(=O)C[N+](C)(C)CCCC(=O)CCOCCOCCOCCOCCOCCOCCNC(=O)CC[C@H](NC(=O)CCCCCCCCCCCCCCCCC(=O)O)C(=O)O)cc2)NC(=O)[C@H]2NC(=O)[C@H](CCCCN(C)C(C)=O)NC(=O)[C@H](Cc3c[nH]c4ccccc34)NC(=O)[C@H]([C@@H](C)O)NC(=O)[C@H](CC)NC(=O)[C@@H](N)C(C)(C)SSC2(C)C)CCCCC1)N(C)C] |
| | cPEG3aCO-Pen(3)-N(N(Me)2)-T-7MeW-K(NMeAc)-Pen(3)-AEF(SP6PEG6gEC18OH)- |
| | 2Nal-THP-K(NMeAc)-N-3Pya-Sar-CON(Me)2 |
| 143 | 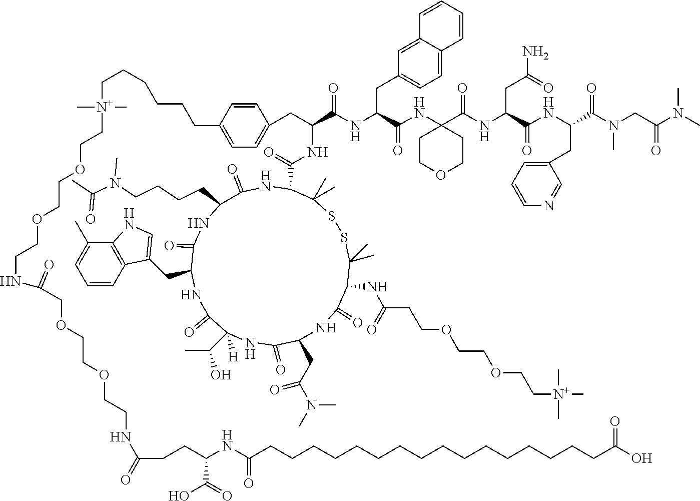 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)CCOCCOCC[N+](C)(C)C)C(C)(C)SSC(C)(C)[C@@H](C(=O)N[C@@H](Cc2ccc(CCCCCC[N+](C)(C)CCOCCOCCNC(=O)COCCOCCNC(=O)CC[C@H](NC(=O)CCCCCCCCCCCCCCCCC(=O)O)C(=O)O)cc2)C(=O)N[C@@H](Cc2ccc3ccccc3c2)C(=O)NC2(C(=O)N[C@@H](CC(N)=O)C(=O)N[C@@H](Cc3cccnc3)C(=O)N(C)CC(=O)N(C)C)CCOCC2)NC1=O] |
| | cPEG3aCO-Pen(3)-N(N(Me)2)-T-7MeW-K(NMeAc)-Pen(3)- |
| | TMAPF(PEG2PEG2gEC18OH)-2Nal-THP-N-3Pya-Sar-CON(Me)2 |
| 144 | 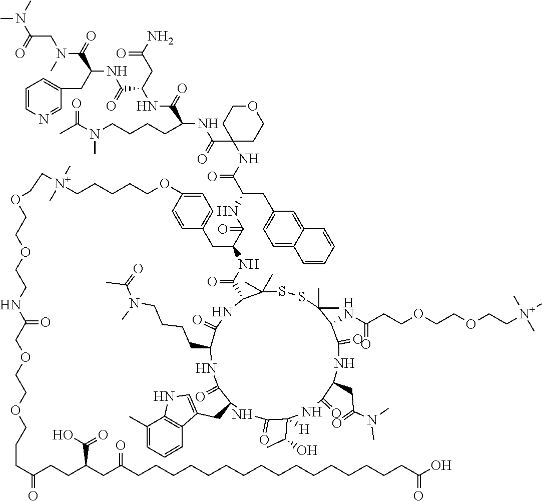 |
| | cPEG3aCO-Pen(3)-N(N(Me)2)-T-7MeW-K(NMeAc)-Pen(3)- |
| | TMAPF(PEG2PEG2gEC18OH)-2Nal-THP-K(NMeAc)-N-3Pya-Sar-CON(Me)2 |
| 145 | 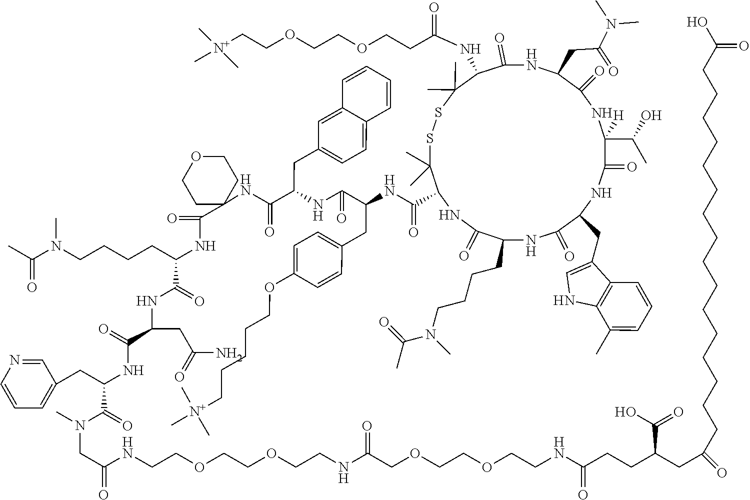 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)CCOCCOCC[N+](C)(C)C)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)NCCOCCOCCNC(=O)COCCOCCNC(=O)CC[C@H](CC(=O)CCCCCCCCCCCCCCCCC(=O)O)C(=O)O)CCOCC2)NC1=O] |
| | cPEG3aCO-Pen(3)-N(N(Me)2)-T-7MeW-K(NMeAc)-Pen(3)-TMAPF-2Nal-THP-K(NMeAc)- |
| | N-3Pya-Sar-CO(PEG2PEG2gEC18OH) |
| 146 | 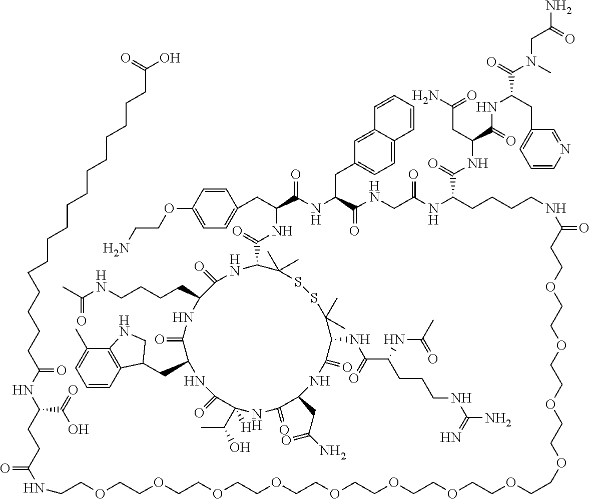 [SMILES: CC(=O)NCCCC[C@@H]1NC(=O)[C@H](CC2CNc3c(C)cccc32)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NCC(=O)N[C@@H](CCCCNC(=O)CCOCCOCCOCCOCCOCCOCCOCCOCCOCCOCCOCCOCCNC(=O)CC[C@H](NC(=O)CCCCCCCCCCCCCCCCC(=O)O)C(=O)O)C(=O)N[C@@H](CC(N)=O)C(=O)N[C@@H](Cc2cccnc2)C(=O)N(C)CC(N)=O)NC1=O] |
| | MeCO-r-Pen(3)-N-T-7MeW-K(Ac)-Pen(3)-AEF-2Nal-THP-K(PEG12gEC18OH)-N-3Pya- |
| | Sar-CONH2 |
| 147 | 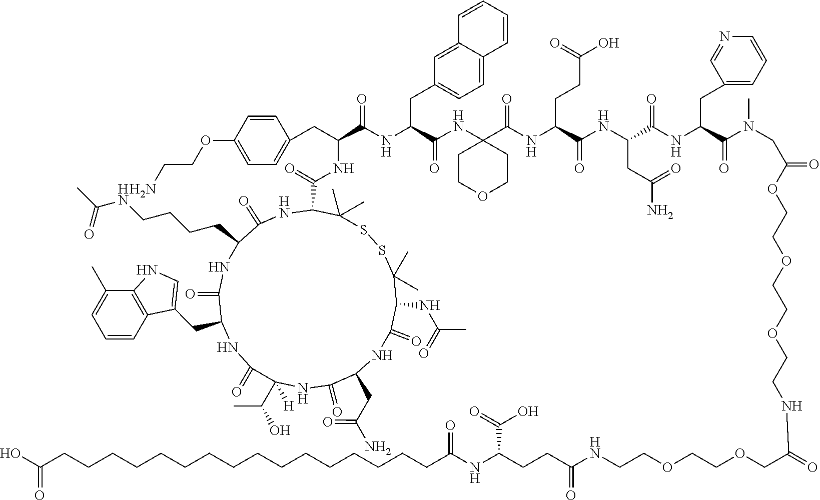 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)OCCOCCOCCNC(=O)COCCOCCNC(=O)CC[C@H](NC(=O)CCCCCCCCCCCCCCCCC(=O)O)C(=O)O)CCOCC2)NC1=O] |
| | MeCO-Pen(3)-N-T-7MeW-K(Ac)-Pen(3)-AEF-2Nal-THP-E-N-3Pya-Sar- |
| | CO(PEG2PEG2gEC18OH) |
| 148 | 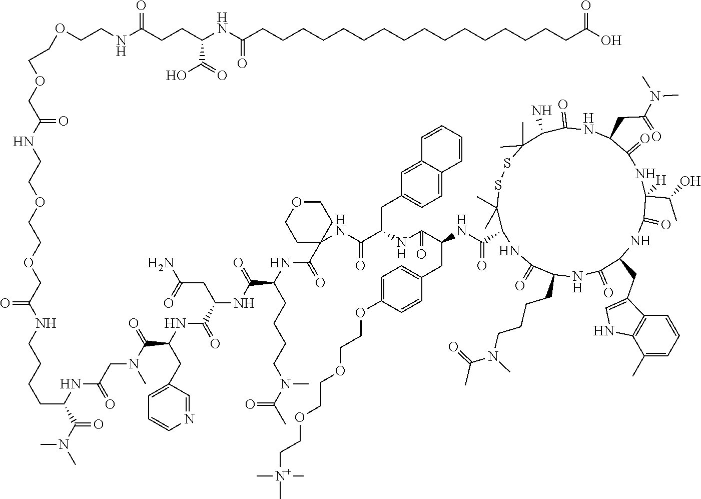 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](N)C(C)(C)SSC(C)(C)[C@@H](C(=O)N[C@@H](Cc2ccc(OCCOCCOCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(=O)N(C)C)CCOCC2)NC1=O] |
| | cPEG3aCO-Pen(3)-N(N(Me)2)-T-7MeW-K(NMeAc)-Pen(3)-APEC3F-2Nal-THP- |
| | K(NMeAc)-N-3Pya-Sar-K(PEG2PEG2gEC18OH)-CON(Me)2 |
| 149 | 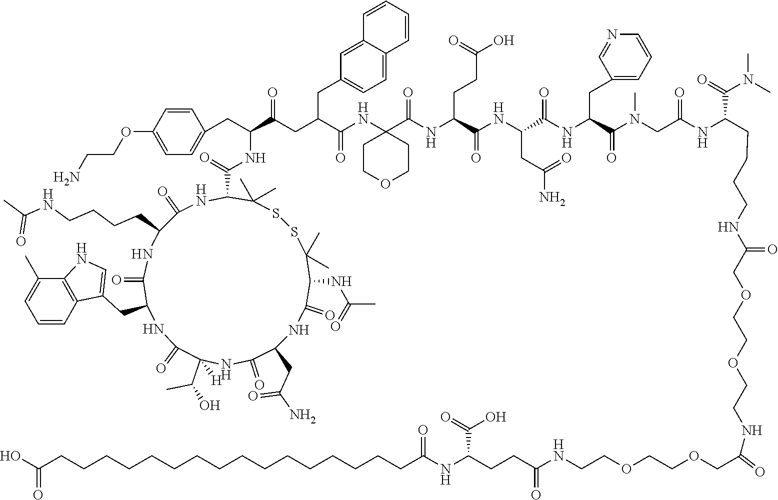 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)CC(Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(=O)N(C)C)CCOCC2)NC1=O] |
| | MeCO-Pen(3)-N-T-7MeW-K(Ac)-Pen(3)-AEF-2Nal-THP-E-N-3Pya-Sar- |
| | K(PEG2PEG2gEC18OH)-CON(Me)2 |
| 150 | 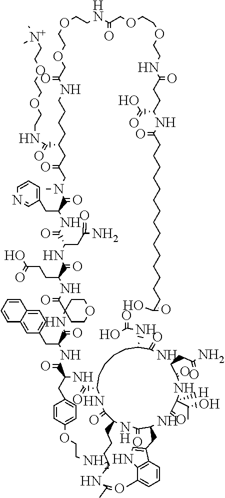 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)O)CCCC[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)C[C@@H](CCCCNC(=O)COCCOCCNC(=O)COCCOCCNC(=O)CC[C@H](NC(=O)CCCCCCCCCCCCCCCC(=O)O)C(=O)O)C(=O)NCCOCCOCC[N+](C)(C)C)CCOCC2)NC1=O] |
| | MeCO-Pen(3)-N-T-7MeW-K(Ac)-Pen(3)-AEF-2Nal-THP-E-N-3Pya-Sar- |
| | K(PEG2PEG2gEC18OH)-CONH(PEG3a) |
| 151 | 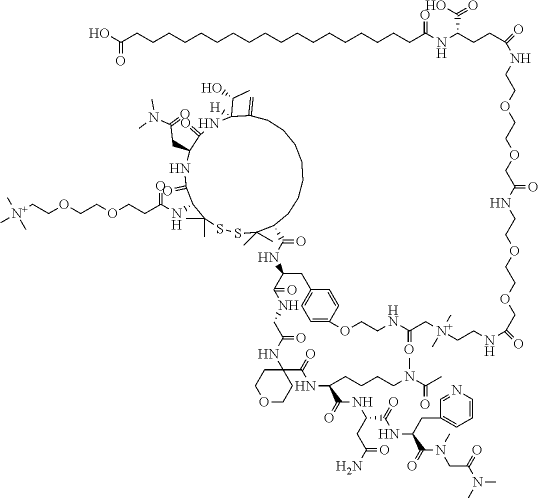 |
| | cPEG3aCO-Pen(3)-N(N(Me)2)-T-7MeW-K(NMeAc)-Pen(3)- |
| | AEF(SP6PEG2PEG2gEC20OH)-2Nal-THP-K(NMeAc)-N-3Pya-Sar-CON(Me)2 |
| 152 | 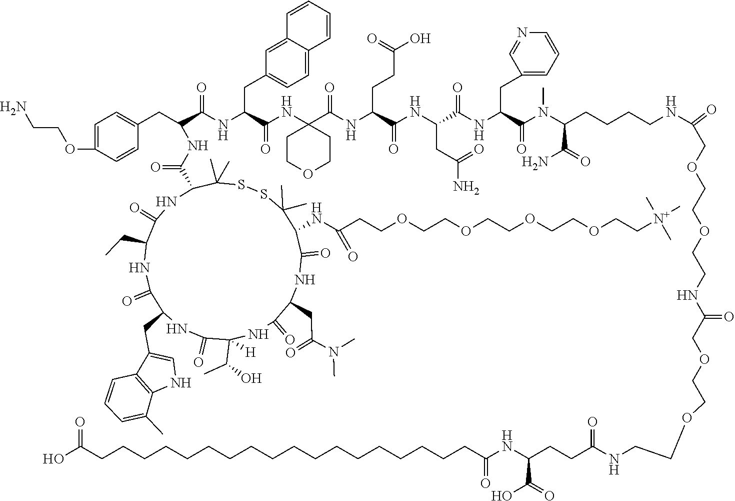 |
| | cPEG5aCO-Pen(3)-N(N(Me)2)-T-7MeW-Dab(NMecPEG5A)-Pen(3)-AEF-2Nal-THP-E-N- |
| | 3Pya-NMeK(PEG2PEG2gEC20OH)-CONH2 |
| 153 | 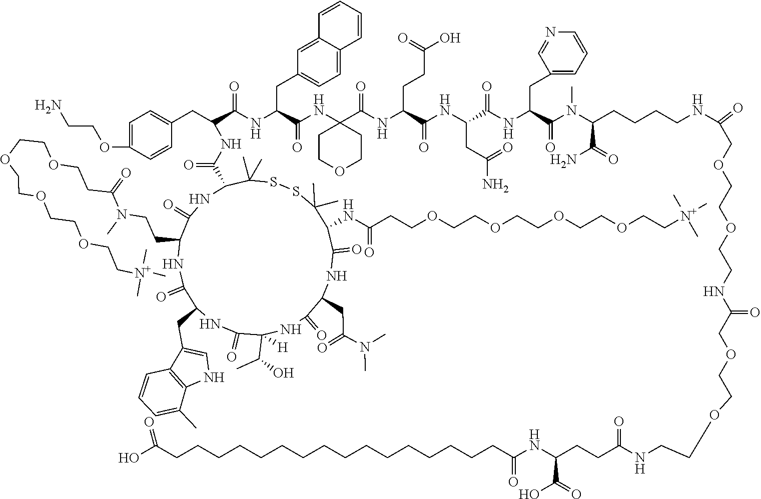 |
| | cPEG5aCO-Pen(3)-N(N(Me)2)-T-7MeW-Dab(NMecPEG5a)-Pen(3)-AEF-2Nal-THP-E-N- |
| | 3Pya-NMeK(PEG2PEG2gEC18OH)-CONH2 |
| 154 | 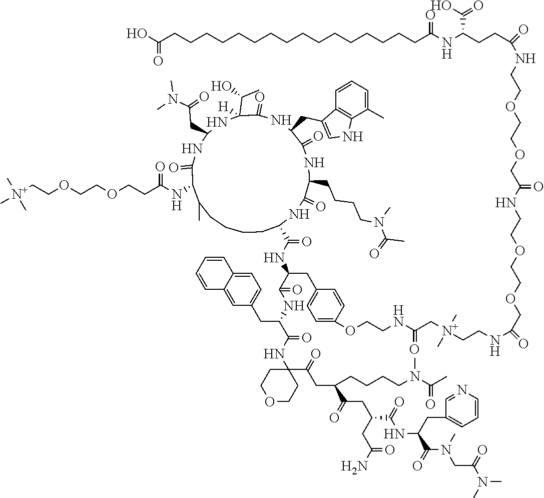 [SMILES: CC(=O)N(C)CCCC[C@H](CC(=O)C1(NC(=O)[C@H](Cc2ccc3ccccc3c2)NC(=O)[C@H](Cc2ccc(OCCNC(=O)C[N+](C)(C)CCNC(=O)COCCOCCNC(=O)COCCOCCNC(=O)CC[C@H](NC(=O)CCCCCCCCCCCCCCCCC(=O)O)C(=O)O)cc2)NC(=O)[C@@H]2CCCCC(C)[C@H](NC(=O)CCOCCOCC[N+](C)(C)C)C(=O)N[C@@H](CC(=O)N(C)C)N[C@@H]([C@@H](C)O)C(=O)N[C@@H](Cc3c[nH]c4c(C)cccc34)C(=O)N[C@@H](CCCCN(C)C(C)=O)C(=O)N2)CCOCC1)C(=O)C[C@@H](CC(N)=O)C(=O)N[C@@H](Cc1cccnc1)C(=O)N(C)CC(=O)N(C)C] |
| | cPEG3aCO-Pen(3)-N(N(Me)2)-T-7MeW-K(NMeAc)-Pen(3)- |
| | AEF(SP6PEG2PEG2gEC18OH)-2Nal-THP-K(NMeAc)-N-3Pya-Sar-CON(Me)2 |
| 155 | 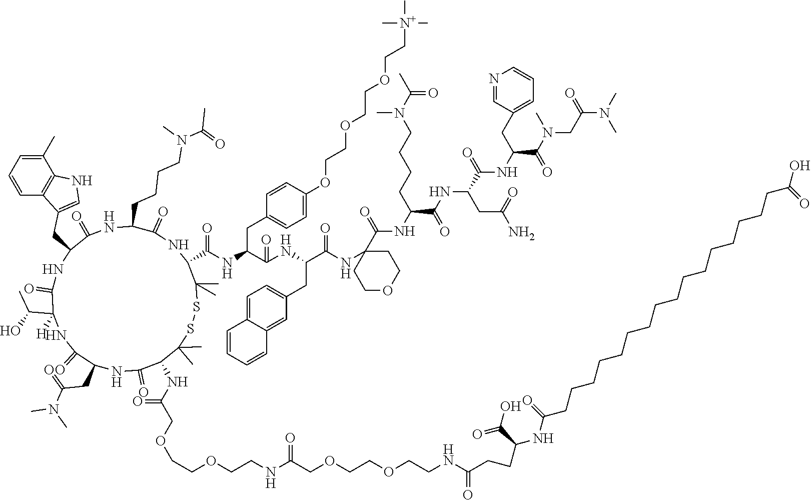 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)COCCOCCNC(=O)COCCOCCNC(=O)CC[C@H](NC(=O)CCCCCCCCCCCCCCCCC(=O)O)C(=O)O)C(C)(C)SSC(C)(C)[C@@H](C(=O)N[C@@H](Cc2ccc(OCCOCCOCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] |
| | HOC18gEPEG2PEG2CO-Pen(3)-N(N(Me)2)-T-7MeW-K(NMeAc)-Pen(3)-APEG3F-2Nal- |
| | THP-K(NMeAc)-N-3Pya-Sar-CON(Me)2 |
| 156 | 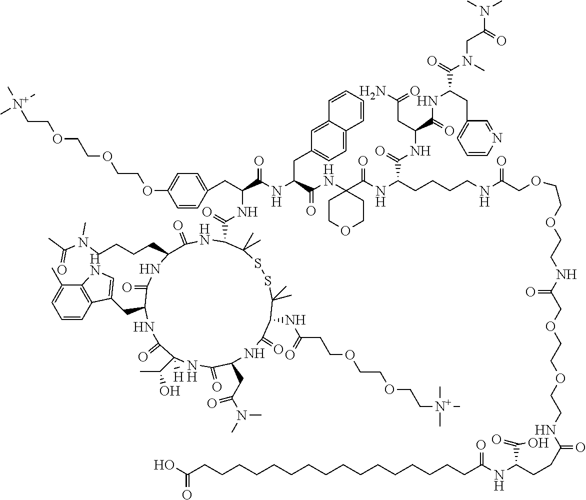 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)CCOCCOCC[N+](C)(C)C)C(C)(C)SSC(C)(C)[C@@H](C(=O)N[C@@H](Cc2ccc(OCCOCCOCC[N+](C)(C)C)cc2)C(=O)N[C@@H](Cc2ccc3ccccc3c2)C(=O)NC2(C(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(=O)N[C@@H](CC(N)=O)C(=O)N[C@@H](Cc3cccnc3)C(=O)N(C)CC(=O)N(C)C)CCOCC2)NC1=O] |
| | cPEG3aCO-Pen(3)-N(N(Me)2)-T-7MeW-K(NMeAc)-Pen(3)-APEG3F-2Nal-THP- |
| | K(PEG2PEG2gEC18OH)-N-3Pya-Sar-CON(Me)2 |
| 157 | 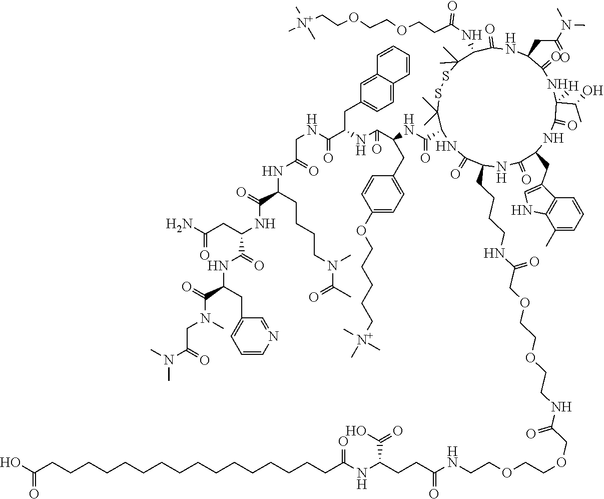 [SMILES: CC(=O)N(C)CCCC[C@H](NC(=O)CNC(=O)[C@H](Cc1ccc2ccccc2c1)NC(=O)[C@H](Cc1ccc(OCCCCC[N+](C)(C)C)cc1)NC(=O)[C@H]1NC(=O)[C@H](CCCCNC(=O)COCCOCCNC(=O)COCCOCCNC(=O)CC[C@H](NC(=O)CCCCCCCCCCCCCCCCC(=O)O)C(=O)O)NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)CCOCCOCC[N+](C)(C)C)C(C)(C)SSC1(C)C)C(=O)N[C@@H](CC(N)=O)C(=O)N[C@@H](Cc1cccnc1)C(=O)N(C)CC(=O)N(C)C] |
| | cPEG3aCO-Pen(3)-N(N(Me)2)-T-7MeW-K(PEG2PEG2gEC18OH)-Pen(3)-APEG3F-2Nal- |
| | THP-K(NMeAc)-N-3Pya-Sar-CON(Me)2 |
| 158 | 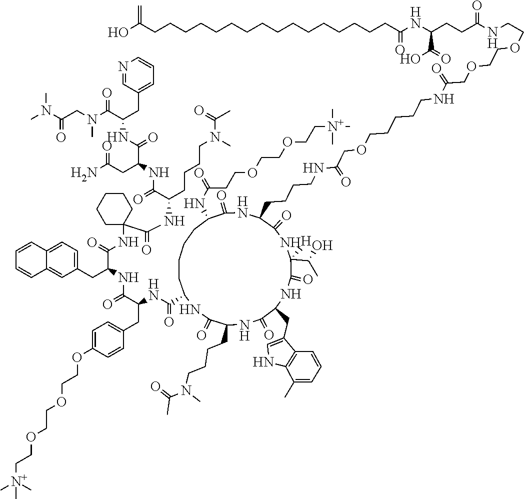 [SMILES: C=C(O)CCCCCCCCCCCCCCCCC(=O)N[C@@H](CCC(=O)NCCOCCOCC(=O)NCCCCCOCC(=O)NCCCC[C@@H]1NC(=O)[C@@H](NC(=O)CCOCCOCC[N+](C)(C)C)CCCC[C@@H](C(=O)N[C@@H](Cc2ccc(OCCOCCOCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCCCC2)NC(=O)[C@H](CCCCN(C)C(C)=O)NC(=O)[C@H](Cc2c[nH]c3c(C)cccc23)NC(=O)[C@H]([C@@H](C)O)NC1=O)C(=O)O] |
| | cPEG3aCO-Pen(3)-K(PEG2PEG2gEC18OH)-T-7MeW-K(NMeAc)-Pen(3)-APEG3F-2Nal- |
| | THP-K(NMeAc)-N-3Pya-Sar-CON(Me)2 |
| 159 | 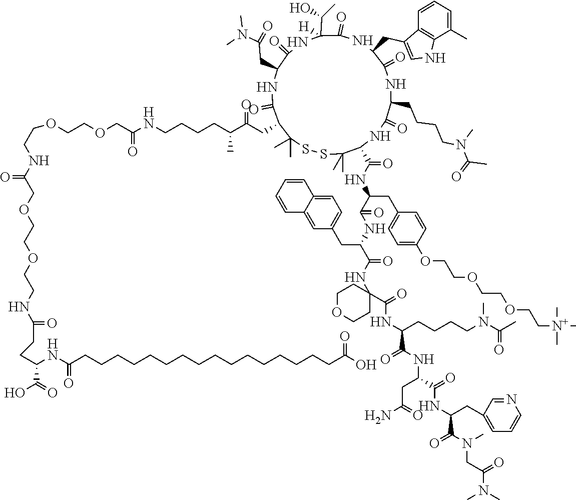 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](CC(=O)[C@H](C)CCCCNC(=O)COCCOCCNC(=O)COCCOCCNC(=O)CC[C@H](NC(=O)CCCCCCCCCCCCCCCCC(=O)O)C(=O)O)C(C)(C)SSC(C)(C)[C@@H](C(=O)N[C@@H](Cc2ccc(OCCOCCOCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] |
| | cPEG3aCO-k(PEG2PEG2gEC18OH)-Pen(3)-N(N(Me)2)-T-7MeW-K(NMeAc)-Pen(3)- |
| | APEG3F-2Nal-THP-K(NMeAc)-N-3Pya-Sar-CON(Me)2 |
| 160 | 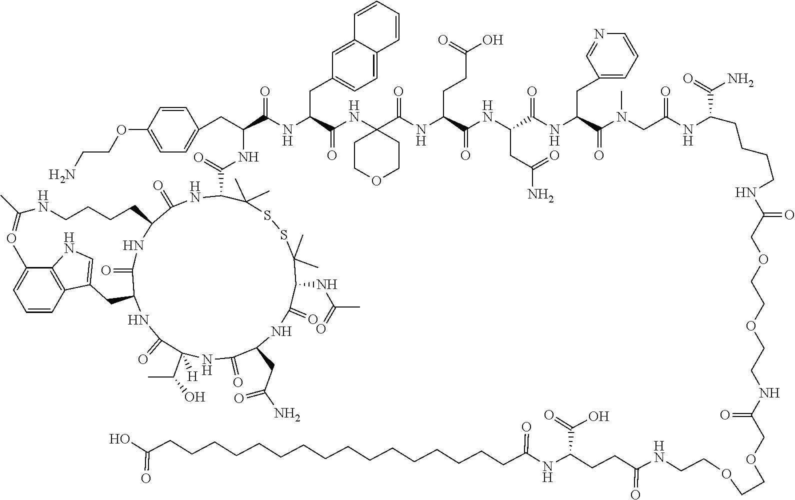 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC2)NC1=O] |
| | cPEG3aCO-r-Pen(3)-N-T-7MeW-K(Ac)-Pen(3)-AEF-2Nal-THP-E-N-3Pya-Sar- |
| | K(PEG2PEG2gEC18OH)-CONH2 |
| 161 | 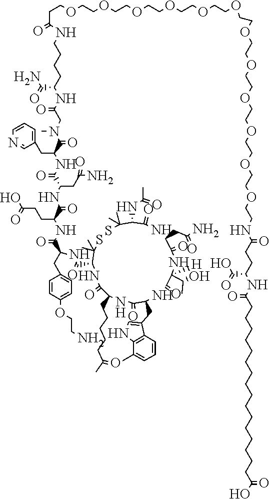 [SMILES: CC(=O)CCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](CCC(=O)O)C(=O)N[C@@H](CC(N)=O)C(=O)N[C@@H](Cc2cccnc2)C(=O)N(C)CC(=O)N[C@@H](CCCCNC(=O)CCOCCOCCOCCOCCOCCOCCOCCOCCOCCOCCOCCOCCNC(=O)CC[C@H](NC(=O)CCCCCCCCCCCCCCCCC(=O)O)C(=O)O)C(N)=O)NC1=O] |
| | MeCO-Pen(3)-N-T-7MeW-K(Ac)-Pen(3)-AEF-2Nal-THP-E-N-3Pya-Sar- |
| | K(PEG12gEC18OH)-CONH2 |
| 162 | 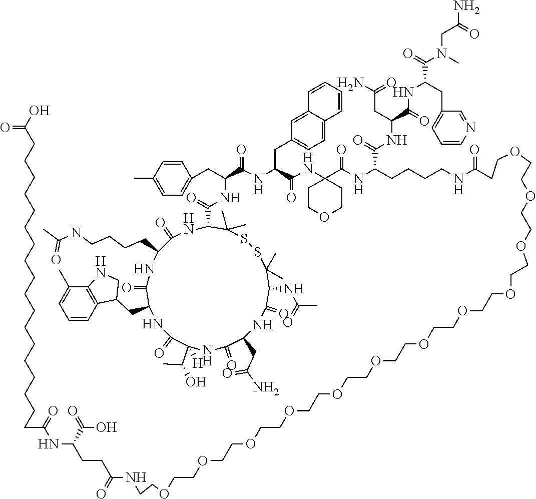 [SMILES: CC(=O)NCCCC[C@@H]1NC(=O)[C@H](CC2CNc3c(C)cccc32)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(C)cc2)C(=O)N[C@@H](Cc2ccc3ccccc3c2)C(=O)NC2(C(=O)N[C@@H](CCCCNC(=O)CCOCCOCCOCCOCCOCCOCCOCCOCCOCCOCCOCCOCCNC(=O)CC[C@H](NC(=O)CCCCCCCCCCCCCCCCCCC(=O)O)C(=O)O)C(=O)N[C@@H](CC(N)=O)C(=O)N[C@@H](Cc3cccnc3)C(=O)N(C)CC(N)=O)CCOCC2)NC1=O] |
| | MeCO-Pen(3)-N-T-7MeW-K(Ac)-Pen(3)-AEF-2Nal-THP-K(PEG12gEC20OH)-N-3Pya-Sar- |
| | CONH2 |

In the peptide sequences shown above, where a number in parenthesis follows a particular residue, that residue is linked to another residue in the sequence that is denoted with the same number. For example, in the sequence MeCO-Pen(3)-N-T-7MeW—K(Ac)-Pen(3)-AEF-2Nal-THP-K(PEG12gEC20OH)—N-3Pya-Sar-CONH2 (SEQ ID NO: 162), the two Pen(3) residues are linked to one another.

### Methods of Synthesis

The compounds described herein may be synthesized by many techniques that are known to those skilled in the art. In some aspects, the present disclosure provides a method of chemically synthesizing a peptide of the present disclosure. In some embodiments, a portion of the peptide is recombinantly synthesized, instead of being chemically synthesized. In some aspects, methods of producing a peptide further include cyclizing the peptide precursor after the constituent subunits have been attached. In particular aspects, cyclization is accomplished via any of the various methods described herein.

The present disclosure further describes synthesis of compounds described herein. In some aspects, one or more of the amino acid residues or amino acid monomers are lipidated and then covalently attached to one another to form a peptide of the disclosure. In some aspects, one or more of the amino acid residues or amino acid monomers are covalently attached to one another and lipidated at an intermediate oligomer stage before attaching additional amino acids and cyclization to form a peptide of the disclosure. In some aspects, a cyclic peptide is synthesized and then lipidated to form a compound of the disclosure. Illustrative synthetic methods are described in the Examples.

### Pharmaceutical Compositions

The present disclosure further relates to a pharmaceutical composition comprising an IL-23R inhibitor described herein. In particular, the present disclosure includes pharmaceutical compositions comprising one or more peptides of the present disclosure and a pharmaceutically acceptable carrier, diluent or excipient. The pharmaceutically acceptable carrier, diluent or excipient may be a solid, semi-solid or liquid filler, diluent, encapsulating material or formulation auxiliary of any type. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like.

The pharmaceutical compositions may be administered orally, parenterally, intracisternally, intravaginally, intraperitoneally, intrarectally, topically (as by powders, ointments, drops, suppository, or transdermal patch), by inhalation (such as intranasal spray), ocularly (such as intraocularly) or buccally. The term “parenteral” as used herein refers to modes of administration which include intravenous, intramuscular, intraperitoneal, intrasternal, subcutaneous, intradermal and intraarticular injection and infusion. Accordingly, in certain embodiments, the compositions are formulated for delivery by any of these routes of administration. A pharmaceutical composition may be formulated for and administered orally. A pharmaceutical composition may be formulated for and administered parenterally.

The IL-23R inhibitors of the present disclosure may be prepared and/or formulated as pharmaceutically acceptable salts and/or other forms thereof or when appropriate in neutral form. Pharmaceutically acceptable salts are non-toxic salts of a neutral form of a compound that possess the desired pharmacological activity of the neutral form. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts can be found in Remington: The Science and Practice of Pharmacy, 21^{st} Edition, Lippincott Williams and Wilkins, Philadelphia, Pa., 2006.

The present disclosure relates to pharmaceutical compositions comprising an IL-23R inhibitor described herein or pharmaceutically acceptable salts, isomers, or a mixture thereof, in which one or more hydrogen atoms attached to a carbon atom may be replaced by a deuterium atom or D. As known in the art, the deuterium atom is a non-radioactive isotope of the hydrogen atom. Such compounds may increase resistance to metabolism, and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism,” Trends Pharmacol. Sci., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled peptides of the present disclosure can generally be prepared by conventional techniques known to those skilled in the art or by processes analogous to those described in the Examples as set out below using an appropriate isotopically-labeled reagent in place of the non-labeled reagent previously employed.

When used in at least one of the treatments or delivery systems described herein, a peptide inhibitor of the present disclosure may be employed in pure form or, where such forms exist, in pharmaceutically acceptable salt form.

The total daily usage of the IL-23R inhibitor and compositions of the present disclosure can be decided by the attending physician within the scope of sound medical judgment. The specific therapeutically effective dose level for any particular subject will depend upon a variety of factors including: a) the disorder being treated and the severity of the disorder; b) activity of the specific compound employed; c) the specific composition employed, the age, body weight, general health, sex and diet of the patient; d) the time of administration, route of administration, and rate of excretion of the specific peptide inhibitor employed; e) the duration of the treatment; f) drugs used in combination or coincidental with the specific peptide inhibitor employed, and like factors well known in the medical arts.

The compositions may conveniently be presented in unit dosage form and can be prepared by any of the methods well known in the art of pharmacy. Techniques and compositions generally are found in Remington's Pharmaceutical Sciences (Mack Publishing Co., Easton, PA). Such methods include the step of bringing into association the active ingredient with the carrier which constitutes one or more accessory ingredients. In general the compositions are prepared by uniformly and intimately bringing into association the active ingredient with liquid carriers or finely divided solid carriers or both, and then, if necessary, shaping the product.

### Non-Invasive Detection of Intestinal Inflammation

The IL-23R inhibitors of the present disclosure may be used for detection, assessment and diagnosis of intestinal inflammation by microPET imaging, wherein the peptide inhibitor is labeled with a chelating group or a detectable label, as part of a non-invasive diagnostic procedure. In certain embodiments, an IL-23R inhibitor of the present disclosure is conjugated with a bifunctional chelator. In certain embodiments, an IL-23R inhibitor of the present disclosure is radiolabeled. The labeled IL-23R inhibitor is then administered to a subject orally or rectally. In certain embodiments, the IL-23R inhibitor is included in drinking water. Following uptake of the IL-23R inhibitor, microPET imaging may be used to visualize inflammation throughout the subject's bowels and digestive track.

### Methods of Treatment and Uses

The present disclosure relates to methods for treating a subject afflicted with a condition or indication associated with IL-23 or IL-23R activity (e.g., activation of the IL-23/IL-23R signaling pathway), wherein the method comprises administering to the subject an IL-23R inhibitor disclosed herein. In one aspect, the present disclosure provides a method for treating a subject afflicted with a condition or indication characterized by aberrant or dysregulated IL-23 or IL-23R activity or signaling, comprising administering to the subject a peptide inhibitor of the present disclosure in an amount sufficient to inhibit (partially or fully) binding of IL-23 to an IL-23R in the subject. The inhibition of IL-23 binding to IL-23R may occur in particular organs or tissues of the subject, e.g., the stomach, small intestine, large intestine/colon, intestinal mucosa, lamina propria, Peyer's Patches, mesenteric lymph nodes, or lymphatic ducts.

The present disclosure relates to methods comprising providing a peptide inhibitor described herein to a subject in need thereof. The subject in need thereof may be a subject that has been diagnosed with or has been determined to be at risk of developing a disease or disorder associated with IL-23/IL-23R. The subject may be a mammal. The subject may be, in particular, a human.

The disease or disorder to be treated by treatment with an IL-23R inhibitor of the present disclosure may be an inflammatory disease or disorder, an autoimmune inflammation diseases or disorder, and/or related disorders, including multiple sclerosis, asthma, rheumatoid arthritis, inflammation of the gut, inflammatory bowel diseases (IBDs), juvenile IBD, adolescent IBD, Crohn's disease, ulcerative colitis, sarcoidosis, Systemic Lupus Erythematosus, ankylosing spondylitis (axial spondyloarthritis), psoriatic arthritis, or psoriasis. In particular, the disease or disorder may be psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, ulcerative colitis, Crohn's disease, Celiac disease (nontropical Sprue), enteropathy associated with seronegative arthropathies, microscopic colitis, collagenous colitis, eosinophilic gastroenteritis/esophagitis, colitis associated with radio- or chemo-therapy, colitis associated with disorders of innate immunity as in leukocyte adhesion deficiency-1, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.

The present disclosure provides a method or use of an IL-23R inhibitor for treating an inflammatory disease or disorder in a subject in need thereof that includes administering to the subject a therapeutically effective amount of an IL-23R inhibitor of the present disclosure, a pharmaceutically acceptable salt thereof, or a composition disclosed herein comprising an IL-23 inhibitor of the present disclosure.

The present disclosure provides a method or use of an IL-23R inhibitor for treating an autoimmune disease or disorder in a subject in need thereof that includes administering to the subject a therapeutically effective amount of an IL-23R inhibitor of the present disclosure, a pharmaceutically acceptable salt thereof, or a composition disclosed herein comprising an IL-23 inhibitor of the present disclosure.

The present disclosure provides a method or use of an IL-23R inhibitor for treating an autoimmune inflammation disease or disorder in a subject in need thereof that includes administering to the subject a therapeutically effective amount of an IL-23R inhibitor of the present disclosure, a pharmaceutically acceptable salt thereof, or a composition disclosed herein comprising an IL-23 inhibitor of the present disclosure.

Suitable inflammatory diseases, autoimmune inflammation diseases, and/or related disorders for treatment with a compound or pharmaceutically acceptable salt thereof, or a composition of the present disclosure, may include, but are not limited to inflammatory bowel disease (IBD), Crohn's disease (CD), ulcerative colitis (UC), psoriasis (PsO), or psoriatic arthritis (PsA) and the like. The inflammatory disease to be treated may be inflammatory bowel disease (IBD), Crohn's disease, or ulcerative colitis. The inflammatory disease to be treated may be selected from psoriasis or psoriatic arthritis. The inflammatory disease to be treated may be psoriasis The inflammatory disease to be treated may be psoriatic arthritis. The inflammatory disease to be treated may be IBD. The inflammatory disease to be treated may be Crohn's disease. The inflammatory disease to be treated may be ulcerative colitis.

Production of IL-23 is enriched in the intestine, where it is believed to play a key role in regulating the balance between tolerance and immunity through T-cell-dependent and T-cell-independent pathways of intestinal inflammation through effects on T-helper 1 (Th1) and Th17-associated cytokines, as well as restraining regulatory T-cell responses in the gut, favoring inflammation. In addition, polymorphisms in the IL-23 receptor (IL-23R) have been associated with susceptibility to inflammatory bowel diseases (IBDs), further establishing the critical role of the IL-23 pathway in intestinal homeostasis. Peptides and methods for specific targeting of the IL-23R from the luminal side of the gut may provide therapeutic benefit to IBD patients suffering from local inflammation of the intestinal tissue.

Accordingly, the present disclosure also provides a method of treating or preventing inflammatory bowel disease (IBD), Crohn's disease (CD), or ulcerative colitis (UC), in a subject in need thereof, said method comprising administering to the subject a therapeutically effective amount of a peptide of the present disclosure, a pharmaceutically acceptable salt thereof, or a pharmaceutical composition described herein comprising an IL-23 inhibitor. In some embodiments, the method is for treating or preventing inflammatory bowel disease (IBD). In some embodiments, the method is for treating or preventing Crohn's disease (CD). In some embodiments, the method is for treating or preventing ulcerative colitis (UC).

Psoriasis, a chronic skin disease affecting about 2%-3% of the general population has been shown to be mediated by the body's T cell inflammatory response mechanisms. IL-23 is one of several interleukins implicated as a key player in the pathogenesis of psoriasis, purportedly by maintaining chronic autoimmune inflammation via the induction of interleukin-17, regulation of T memory cells, and activation of macrophages. Expression of IL-23 and IL-23R has been shown to be increased in tissues of patients with psoriasis, and antibodies that neutralize IL-23 showed TL-23-dependent inhibition of psoriasis development in animal models of psoriasis. Orally bioavailable peptide inhibitors of IL-23 may provide both a non-steroidal treatment option for patients with mild to moderate psoriasis and treatment for moderate to severe psoriasis that does not require delivery by infusion.

Accordingly, the present disclosure also provides a method of treating or preventing psoriasis (PsO) or psoriatic arthritis (PsA) in a subject in need thereof, said method comprising administering to the subject a therapeutically effective amount of a peptide of the present disclosure, a pharmaceutically acceptable salt thereof, or a pharmaceutical composition described herein comprising an IL-23 inhibitor. In some embodiments, the method is for treating or preventing psoriasis (PsO). In some embodiments, the method is for treating or preventing psoriatic arthritis (PsA).

The present disclosure further relates to a method of selectively inhibiting IL-23 or IL-23R signaling (or the binding of IL-23 to IL-23R) in a subject (e.g., in a subject in need thereof), comprising administering to the subject a peptide inhibitor of the IL-23R described herein. In some embodiments, the present disclosure includes and provides a method of selectively inhibiting IL-23 or IL-23R signaling (or the binding of IL-23 to IL-23R) in the GI tract of a subject (e.g., a subject in need thereof), comprising providing to the subject a peptide inhibitor of the IL-23R of the present disclosure by oral administration. The exposure of GI tissues (e.g., small intestine or colon) to the administered peptide inhibitor may be at least 10-fold, at least 20-fold, at least 50-fold, or at least 100-fold greater than the exposure (level) in the blood. In particular embodiments, the present disclosure includes a method of selectively inhibiting IL23 or IL23R signaling (or the binding of IL23 to IL23R) in the GI tract of a subject (e.g., a subject in need thereof), comprising providing to the subject a peptide inhibitor, wherein the peptide inhibitor does not block the interaction between IL-6 and IL-6R or antagonize the IL-12 signaling pathway. In a further related embodiment, the present disclosure provides a method of inhibiting GI inflammation and/or neutrophil infiltration to the GI, comprising providing to a subject in need thereof a peptide inhibitor of the present disclosure. In some embodiments, methods of the present disclosure comprise providing a peptide inhibitor of the present disclosure (i.e., a first therapeutic agent) to a subject (e.g., a subject in need thereof) in combination with a second therapeutic agent. In certain embodiments, the second therapeutic agent is provided to the subject before and/or simultaneously with and/or after the peptide inhibitor is administered to the subject. In particular embodiments, the second therapeutic agent is an anti-inflammatory agent. In certain embodiments, the second therapeutic agent is a non-steroidal anti-inflammatory drug, steroid, or immune modulating agent. In certain embodiments, the method comprises administering to the subject a third therapeutic agent. In certain embodiments, the second therapeutic agent is an antibody that binds IL-23 or IL-23R.

The present disclosure also relates to methods of inhibiting IL-23 binding to an IL-23R on a cell, comprising contacting the IL-23R with a peptide inhibitor of the receptor disclosed herein. The cell may be a mammalian cell. The method may be performed in vitro or in vivo. Inhibition of binding may be determined by a variety of routine experimental methods and assays known in the art.

The present disclosure relates to methods of inhibiting IL-23 signaling by a cell, comprising contacting the IL-23R with a peptide inhibitor described herein. In certain embodiments, the cell is a mammalian cell. In particular embodiments, the method is performed in vitro or in vivo. In particular embodiments, the inhibition of IL-23 signaling may be determined by measuring changes in phospho-STAT3 levels in the cell.

### EXAMPLES

The following examples are not intended to limit the scope of the present disclosure, but rather to provide guidance to the skilled artisan to prepare and use the peptides, compositions, and methods of the present disclosure. While particular aspects of the present disclosure are described, the skilled artisan will appreciate that various changes and modifications can be made without departing from the spirit and scope of the disclosure.

Some abbreviations useful in describing the disclosure are defined below in the following tables.

Herein and throughout the application, the following abbreviations may be used.

| Abbreviation | Definition |
|---|---|
| ACN | acetonitrile |
| Boc | tert-butoxy-carbonyl |
| carnitine- | (R)-4-(tert-butoxy)-2-(3-(hydroxy-l2- |
| succinate, carn | methoxy)propanamido)-N,N,N-trimethyl-4-oxobutan- |
| | 1-aminium |
| DCM | dichloromethane |
| Dde | N-(1-(4,4-dimethyl-2,6-dioxocyclohexylidene)ethyl |
| DIC | N,N′-diisopropylcarbodiimide |
| DIPEA | N,N-diisopropylethylamine |
| DMF | N,N-dimethylformamide |
| DMSO | dimethyl sulfoxide |
| DTT | dithiothreitol |
| ESI | electrospray ionization |
| Et₂O | diethyl ether |
| FMOC or | ((9H-fluoren-9-yl)methoxy)carbonyl |
| Fmoc | |
| h | hour |
| HATU | 1-[bis(dimethylamino)methylene]-1H-1,2,3-triazolo[4,5- |
| | b]pyridinium 3-oxide hexafluorophosphate |
| HOAT or | 1-hydroxy-7-azabenzotriazole |
| HOAt | |
| HPLC | high-performance liquid chromatography |
| MeOH | methanol |
| min | minute |
| MS | mass spectrometry |
| MTBE | methyl tert-butyl ether |
| MW | microwave |
| NDMBA | 1,3-dimethylbarbituric acid |
| Oxyma | ethyl cyanohydroxyiminoacetate |
| RT | room temperature |
| SPPS | solid-phase peptide synthesis |
| TBAF | tetrabutylammonium fluoride |
| TEA | triethylamine |
| TFA | trifluoroacetic acid |
| TFE | tetrafluoroethylene |
| THF | tetrahydrofuran |
| TIPS | triisopropyl silane |
| TIS | triisopropyl silane |
| UPLC | ultra performance liquid chromatography |

TABLE 5
N-Terminal Modification Abbreviations

| Abbreviation | Structure |
|---|---|
| 5cpaCO | 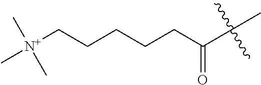 [SMILES: *C(=O)CCCCC[N+](C)(C)C] |
| 5Ava | 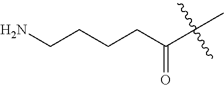 [SMILES: *C(=O)CCCCN] |
| | 5-aminovaleric acid |
| 6Ahx | 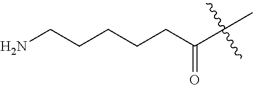 [SMILES: *C(=O)CCCCCN] |
| | 6-aminohexanoic acid |
| 7Ahp | 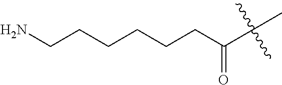 [SMILES: *C(=O)CCCCCCN] |
| | 7-aminoheptanoic acid |
| 8Aoc | 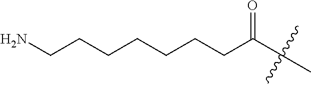 [SMILES: *C(=O)CCCCCCCN] |
| | 8-aminooctanoic acid |
| AEEP-Z_{lipid} | 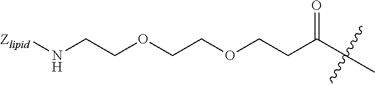 |
| BiotinPEG4CO | 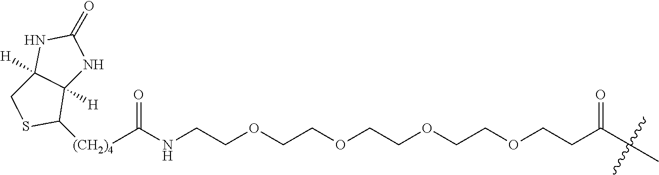 |
| C12gEPEG2PEG2CO | 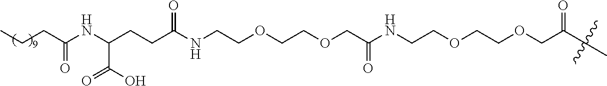 |
| C14gEPEG2PEG2CO | 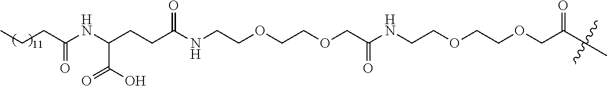 |
| C6XgEPEG2PEG2CO | 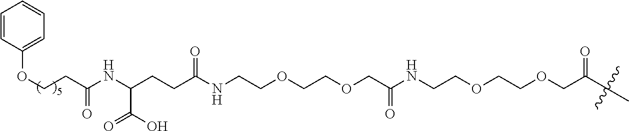 |
| cPEG3aCO | 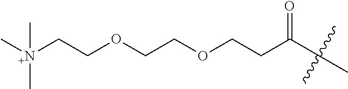 [SMILES: *C(=O)CCOCCOCC[N+](C)(C)C] |
| cPEG5aCO | 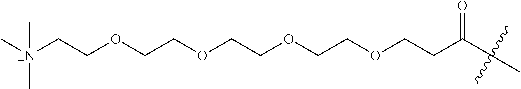 [SMILES: *C(=O)CCOCCOCCOCCOCC[N+](C)(C)C] |
| FITCPEG2CO | 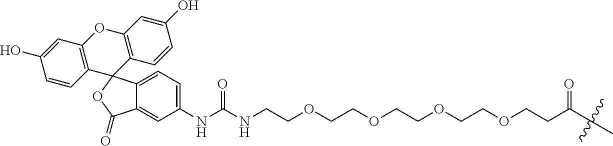 [SMILES: *C(=O)CCOCCOCCOCCOCCNC(=O)Nc1ccc2c(c1)C(=O)OC21c2ccc(O)cc2Oc2cc(O)ccc21] |
| HOC18gEPEG12CO | 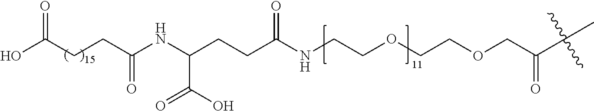 |
| HOC10gEPEG2COPEG2CO | 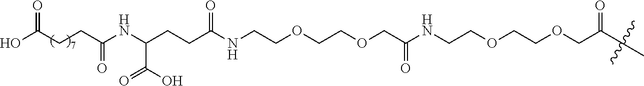 |
| PEG2PEG2-gEC16OH, HOC16gEPEG2PEG2 | 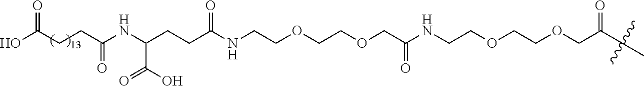 |
| HOC18gEPEG2PEG2CO, HOC18gEPEG2PEG2 | 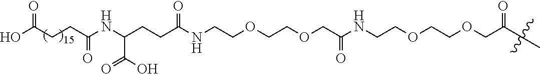 |
| HOC18gEPEG2PEG2COSP6 | 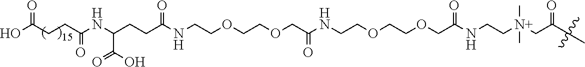 |
| HOC18gEPEG2SP6PEG2CO | 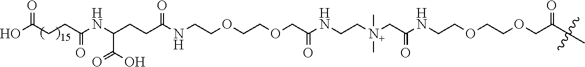 |
| HOC18gEPEG6CO | 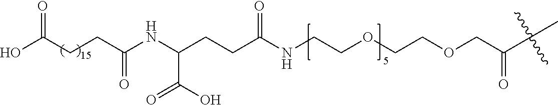 |
| HOC20gEPEG12CO | 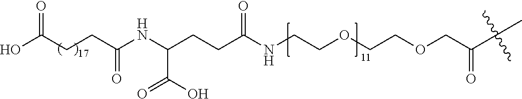 |
| HOC20gEPEG2PEG2CO | 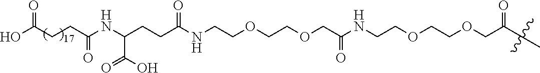 |
| MeCO | 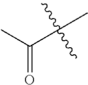 [SMILES: *C(C)=O] |
| succiniccarn | 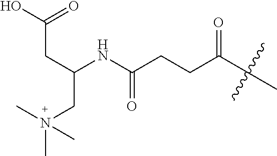 |
| PEG2_Palm | 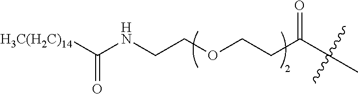 |
| PEG2_Lauryl | 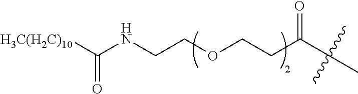 |
| PEG4_Palm | 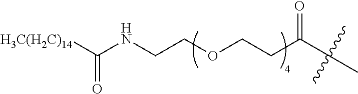 |
| PEG4_Lauryl | 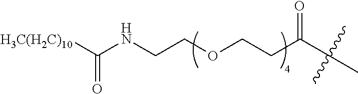 |
| PEG4_Capryl | 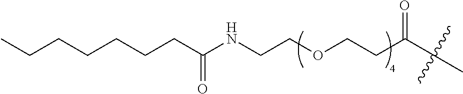 |
| PEG4_Hexyl | 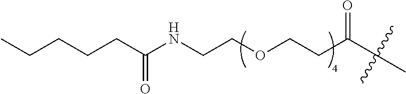 |

TABLE 6
C-Terminal Modification Abbreviations

| Abbreviation | Structure |
|---|---|
| CONH2 | 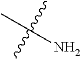 [SMILES: *N] |
| CONH-Z_{peg} | 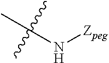 |
| CONH-Z_{lipid} | 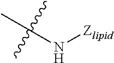 |
| CON(Me)2 | 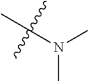 [SMILES: *N(C)C] |

The amino acid structures provided in Table 7A, below, are presented without stereochemical indicators at the alpha carbon; however, it is to be understood that these amino acids occur as either the L-amino acid or the D-amino acid. For example, “Dap” may be present in the peptides of the instant disclosure as the L-stereoisomer:

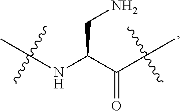 [SMILES: *N[C@@H](CN)C(*)=O]

or as the D-stereoisomer (e.g., when referred to as “dap,” “dDap,” or “D-Dap”):

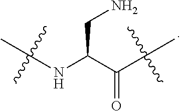 [SMILES: *N[C@@H](CN)C(*)=O]

TABLE 7A
Monomer Abbreviations

| Abbreviation | Structure |
|---|---|
| 2Nal | 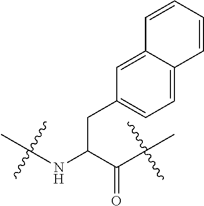 [SMILES: *NC(Cc1ccc2ccccc2c1)C(*)=O] |
| 3Pya | 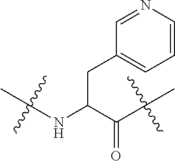 [SMILES: *NC(Cc1cccnc1)C(*)=O] |
| 4AminoPro | 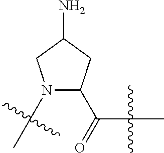 [SMILES: *C(=O)C1CC(N)CN1*] |
| 4RAminoPro | 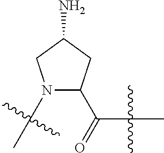 [SMILES: *C(=O)C1C[C@@H](N)CN1*] |
| 4SAminoPro | 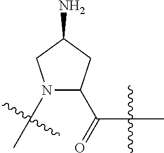 [SMILES: *C(=O)C1C[C@H](N)CN1*] |
| 7MeW | 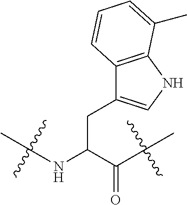 [SMILES: *NC(Cc1c[nH]c2c(C)cccc12)C(*)=O] |
| Abu | 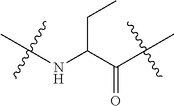 [SMILES: *NC(CC)C(*)=O] |
| | 2-aminobutyric acid |
| AEF | 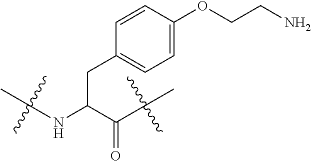 [SMILES: *NC(Cc1ccc(OCCN)cc1)C(*)=O] |
| AEF(d) | 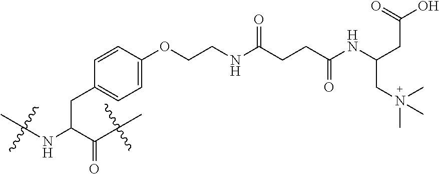 |
| AEF-Z_{peg} | 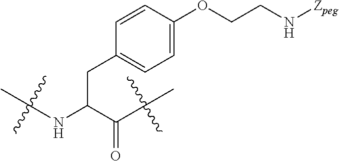 |
| AEF-Z_{lipid} | 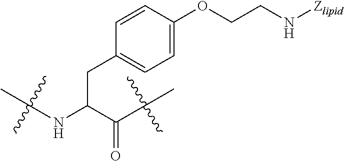 |
| aG | 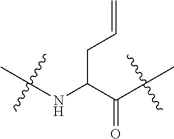 [SMILES: *NC(CC=C)C(*)=O] |
| aMeC | 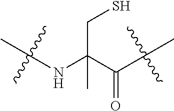 [SMILES: *NC(C)(CS)C(*)=O] |
| APEG3F | 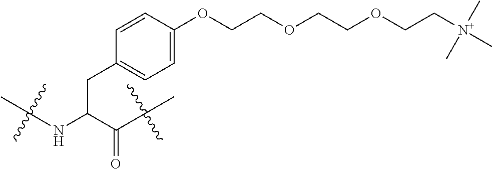 [SMILES: *NC(Cc1ccc(OCCOCCOCC[N+](C)(C)C)cc1)C(*)=O] |
| Api | 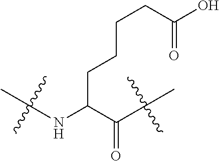 [SMILES: *NC(CCCCC(=O)O)C(*)=O] |
| Dab | 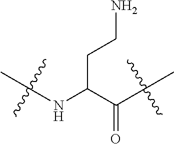 [SMILES: *NC(CCN)C(*)=O] |
| | 2,4-diaminobutyric acid |
| Dab(NMecarn) | 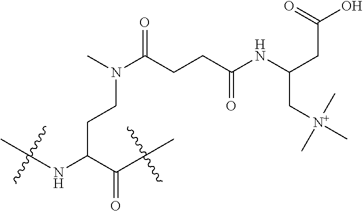 [SMILES: *NC(CCN(C)C(=O)CCC(=O)NC(CC(=O)O)C[N+](C)(C)C)C(*)=O] |
| Dab-Z_{lipid} | 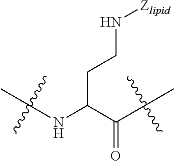 |
| Dab-Z_{peg} | 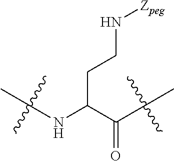 |
| Dap | 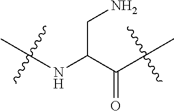 [SMILES: *NC(CN)C(*)=O] |
| | 2,3-diaminopropionic acid |
| Dap(N3) | 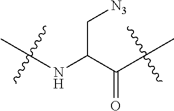 [SMILES: *NC(CN=[N+]=[N-])C(*)=O] |
| hA | 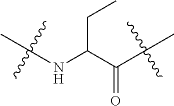 [SMILES: *NC(CC)C(*)=O] |
| hE or hGlu | 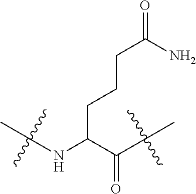 [SMILES: *NC(CCCC(N)=O)C(*)=O] |
| hK or hLys | 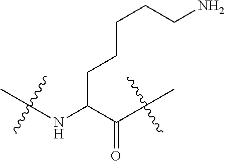 [SMILES: *NC(CCCCCN)C(*)=O] |
| E(OAll) | 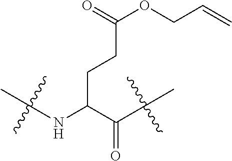 [SMILES: *NC(CCC(=O)OCC=C)C(*)=O] |
| K(Ac) | 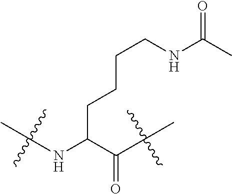 [SMILES: *NC(CCCCNC(C)=O)C(*)=O] |
| K(d) | 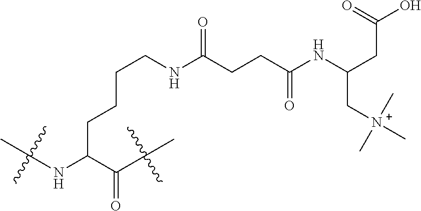 [SMILES: *NC(CCCCNC(=O)CCC(=O)NC(CC(=O)O)C[N+](C)(C)C)C(*)=O] |
| K(NMeAc) | 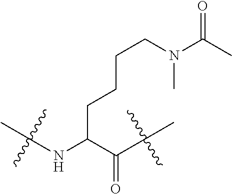 [SMILES: *NC(CCCCN(C)C(C)=O)C(*)=O] |
| K-Z_{lipid} | 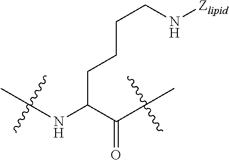 |
| K-Z_{peg} | 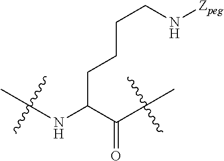 |
| K(NMe)-Z_{lipid} | 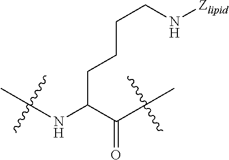 |
| K(NMe)-Z_{peg} | 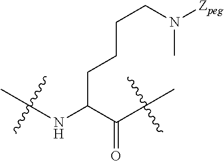 |
| NMeK-Z_{lipid} | 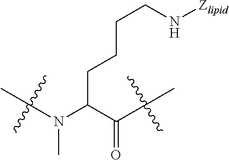 |
| NMeK-Z_{peg} | 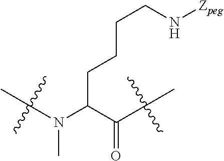 |
| N(NMe2) | 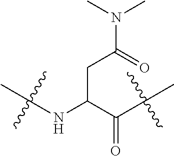 [SMILES: *NC(CC(=O)N(C)C)C(*)=O] |
| NMeK(d) | 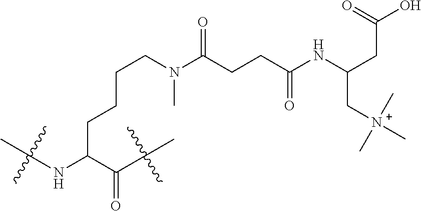 [SMILES: *NC(CCCCN(C)C(=O)CCC(=O)NC(CC(=O)O)C[N+](C)(C)C)C(*)=O] |
| Orn | 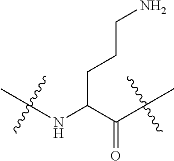 [SMILES: *NC(CCCN)C(*)=O] |
| Pen | 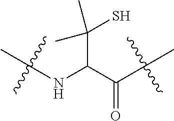 [SMILES: *NC(C(*)=O)C(C)(C)S] |
| Pen(mXy1) | 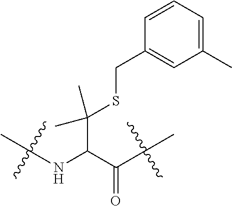 [SMILES: *NC(C(*)=O)C(C)(C)SCc1cccc(C)c1] |
| Pen(oXyl) | 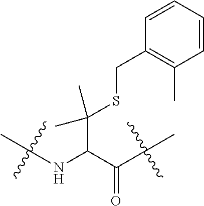 [SMILES: *NC(C(*)=O)C(C)(C)SCc1ccccc1C] |
| Pen(pXy1) | 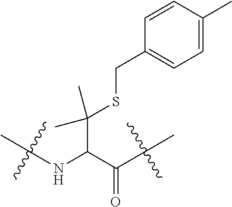 [SMILES: *NC(C(*)=O)C(C)(C)SCc1ccc(C)cc1] |
| Pra | 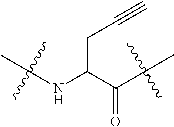 [SMILES: *NC(CC#C)C(*)=O] |
| S5Me | 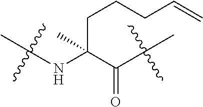 [SMILES: *N[C@@](C)(CCCC=C)C(*)=O] |
| R5Me | 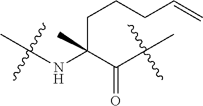 [SMILES: *N[C@](C)(CCCC=C)C(*)=O] |
| S5H | 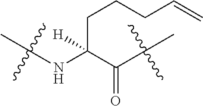 [SMILES: *N[C@@H](CCCC=C)C(*)=O] |
| R5H | 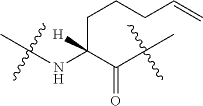 [SMILES: *N[C@H](CCCC=C)C(*)=O] |
| Sar | 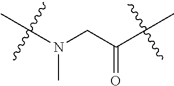 [SMILES: *C(=O)CN(*)C] |
| SP6 | 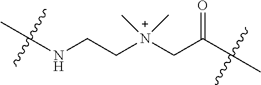 [SMILES: *NCC[N+](C)(C)CC(*)=O] |
| THP | 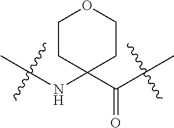 [SMILES: *NC1(C(*)=O)CCOCC1] |
| TMAPF | 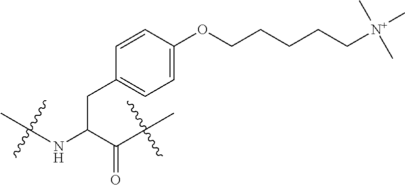 [SMILES: *NC(Cc1ccc(OCCCCC[N+](C)(C)C)cc1)C(*)=O] |
| TMAPF-Z_{lipid} | 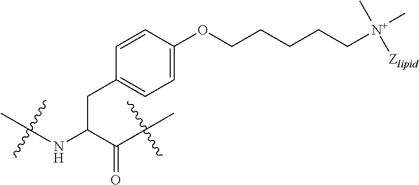 |

TABLE 7B
Lipidated Monomer Abbreviations

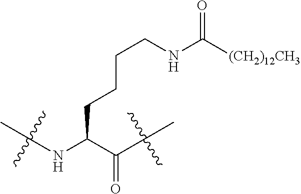

K(C14)

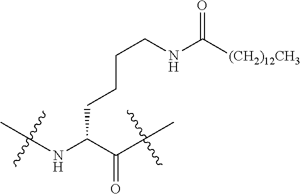

k(C14)

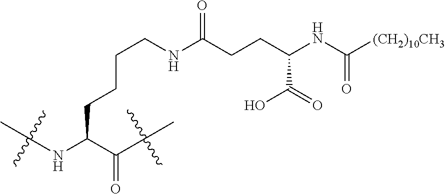

K(gEC12)

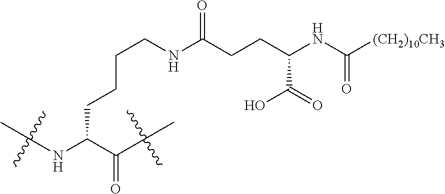

k(gEC12)

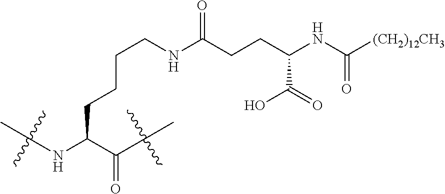

K(gEC14)

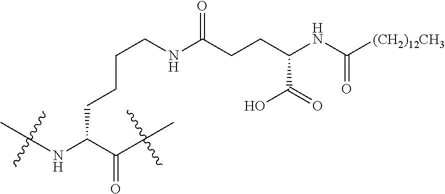

k(gEC14)

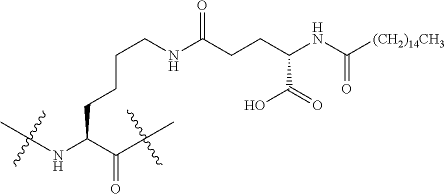

K(gEC16), K(IsoGlu_Palm)

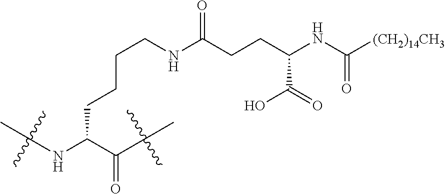

k(gEC16), k(IsoGlu_Palm)

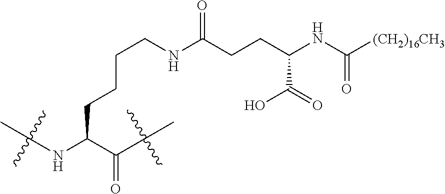

K(gEC18)

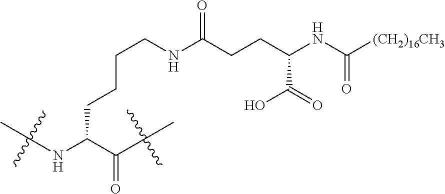

k(gEC18)

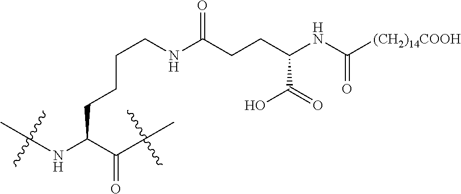

K(gEC16OH)

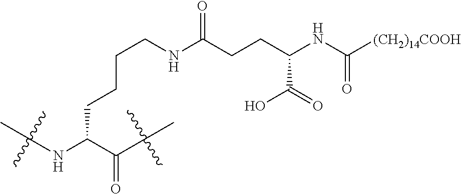

k(gEC16OH)

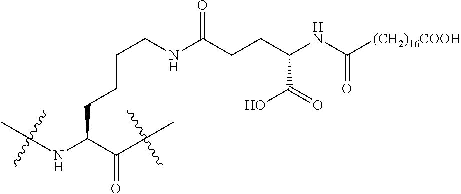

K(gEC18OH)

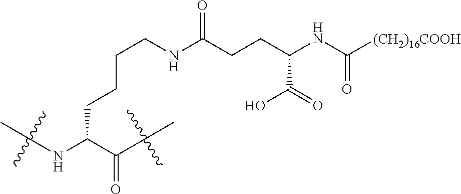

k(gEC18OH)

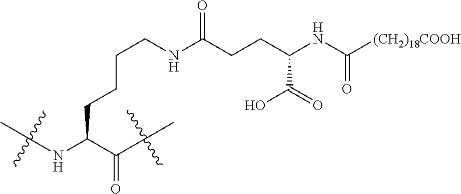

K(gEC20OH)

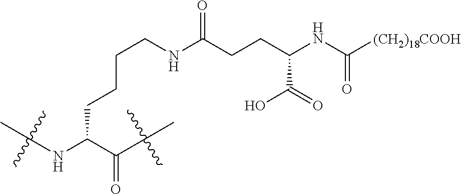

k(gEC20OH)

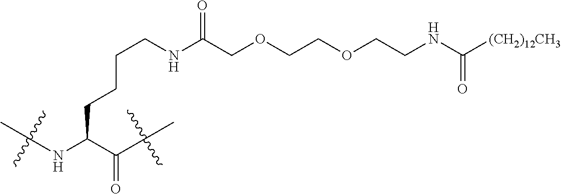

K(PEG2C14)

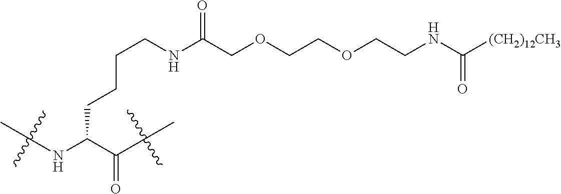

k(PEG2C14)

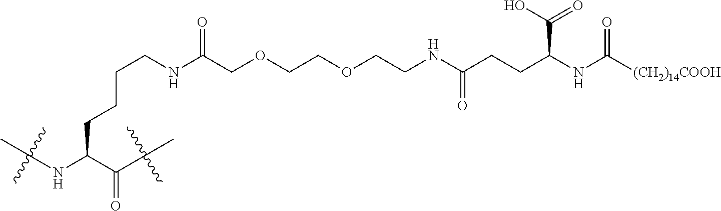

K(PEG2gEC16OH)

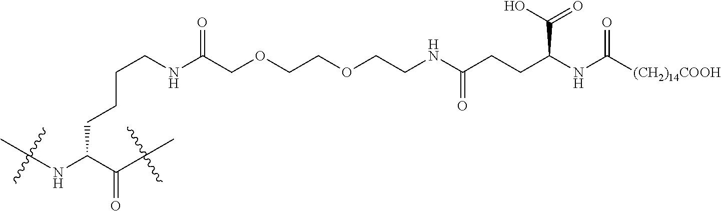

k(PEG2gEC16OH)

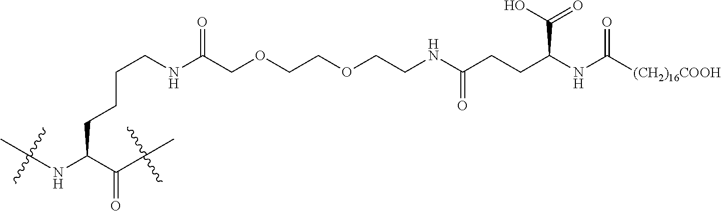

K(PEG2gEC18OH)

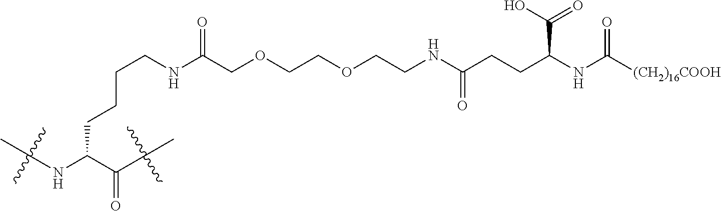

k(PEG2gEC18OH)

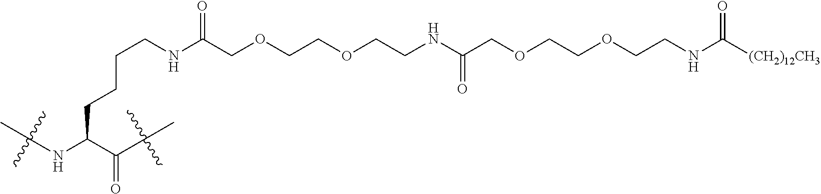

K(PEG2PEG2C14)

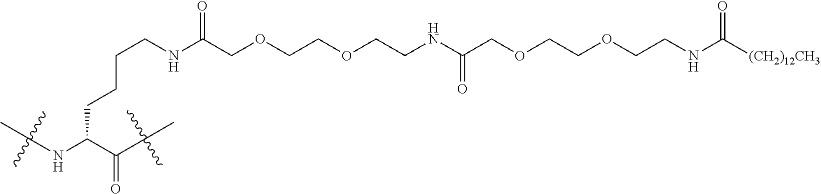

k(PEG2PEG2C14)

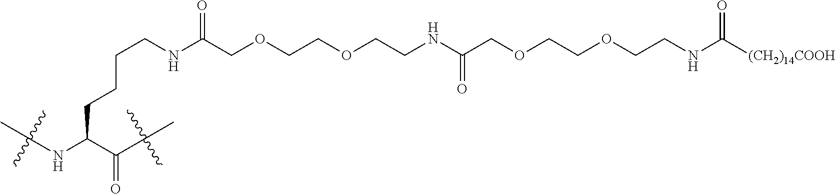

K(PEG2PEG2C16OH)

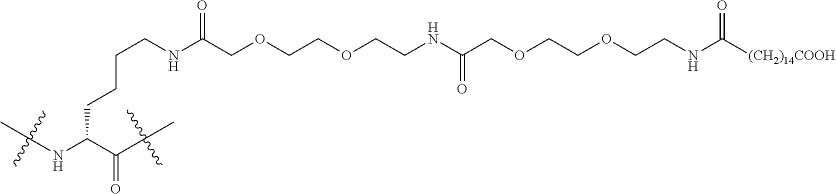

k(PEG2PEG2C16OH)

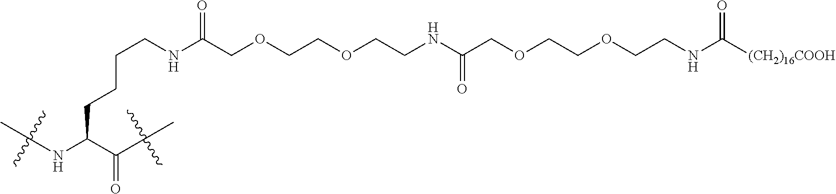

K(PEG2PEG2C18OH)

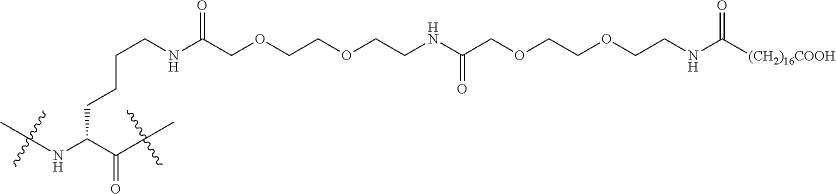

k(PEG2PEG2C18OH)

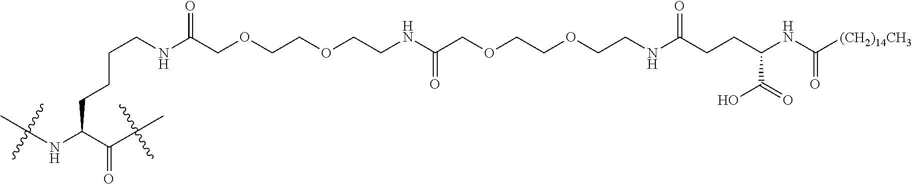

K(PEG2PEG2gEC16)

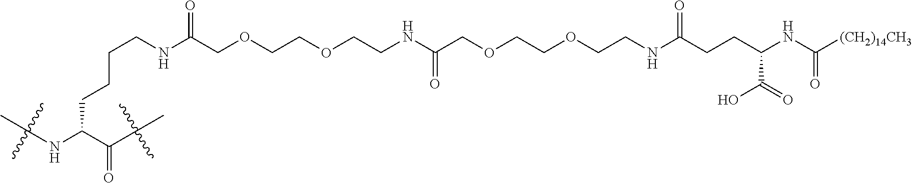

k(PEG2PEG2gEC16)

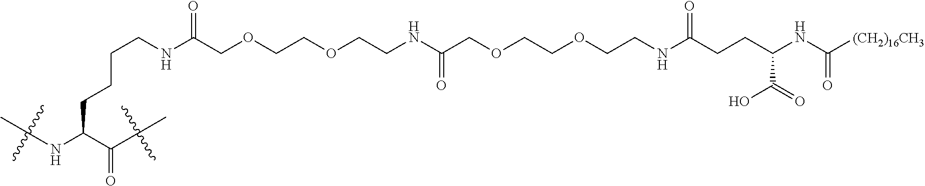

K(PEG2PEG2gEC18)

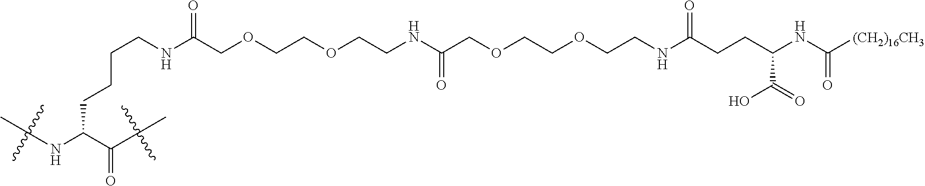

k(PEG2PEG2gEC18)

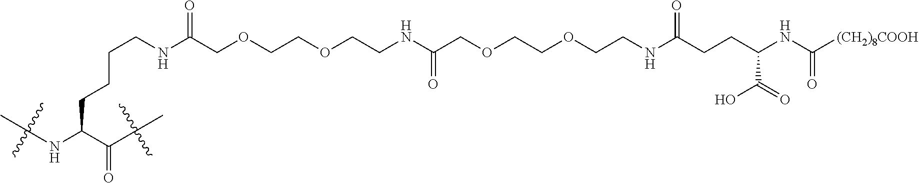

K(PEG2PEG2gEC10OH)

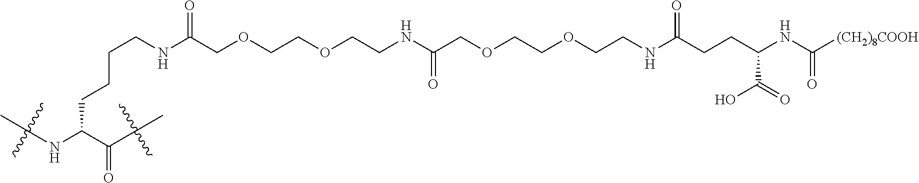

k(PEG2PEG2gEC10OH)

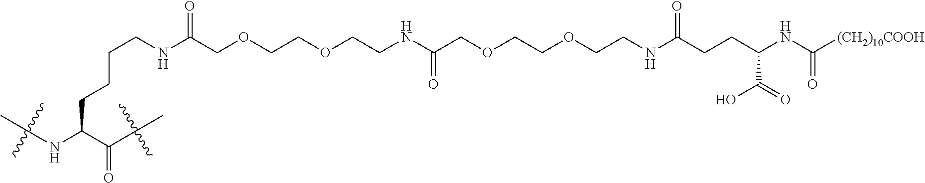

K(PEG2PEG2gEC12)

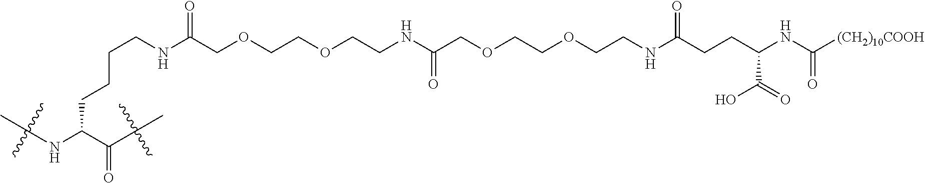

k(PEG2PEG2gEC12)

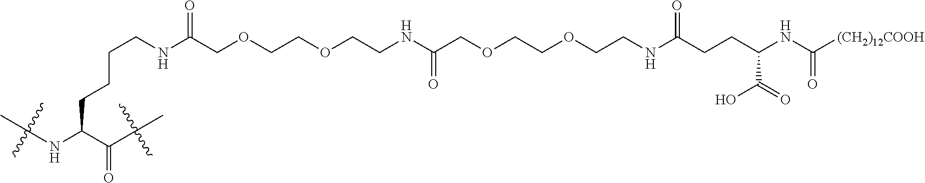

K(PEG2PEG2gEC14)

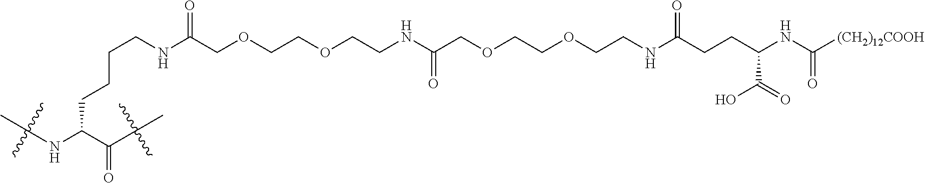

k(PEG2PEG2gEC14)

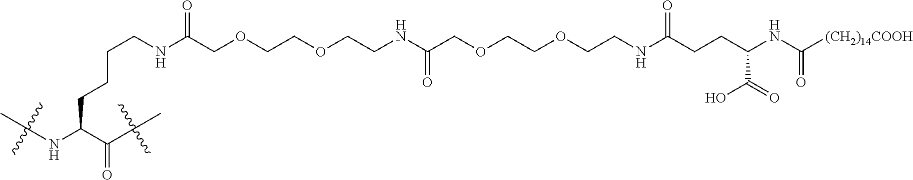

K(PEG2PEG2gEC16OH), K(1PEG21PEG2IsoGluC16Diacid)

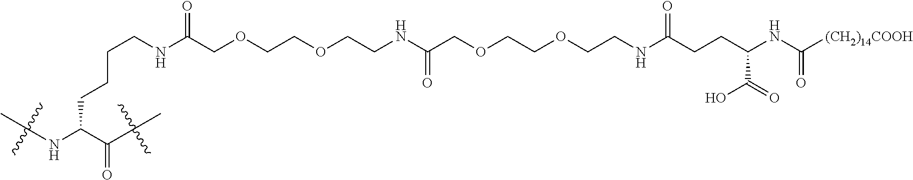

k(PEG2PEG2gEC16OH)

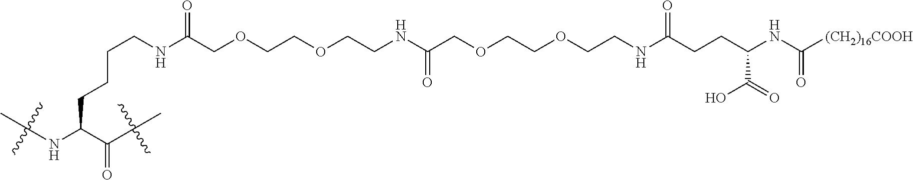

K(PEG2PEG2gEC18OH), K(1PEG21PEG2IsoGluC18Diacid),

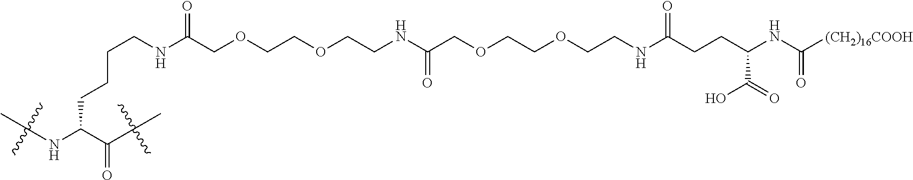

k(PEG2PEG2gEC18OH), k(IsoGlu_C18_Diacid)

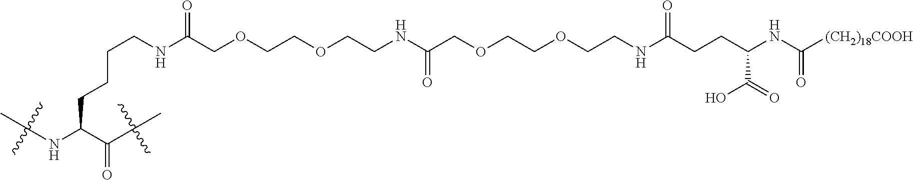

K(PEG2PEG2gEC20OH)

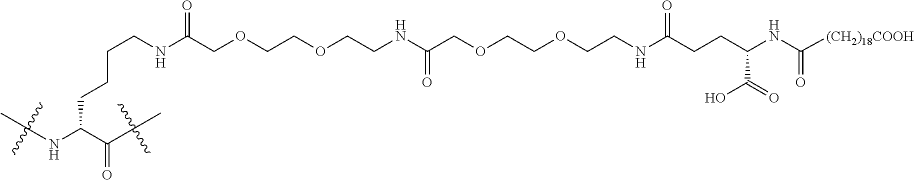

k(PEG2PEG2gEC20OH)

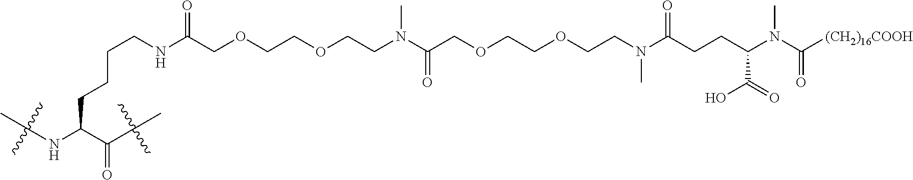

K(PEG2NMePEG2NMegENMeC18OH)

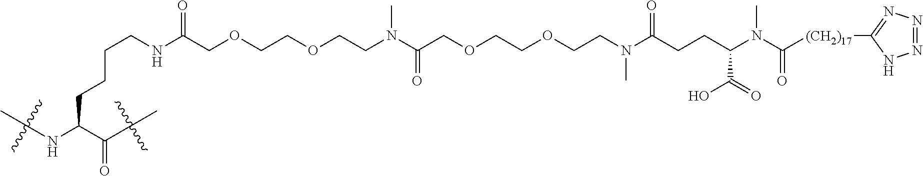

K(PEG2NMePEG2NMegENMeC18Tetrazole)

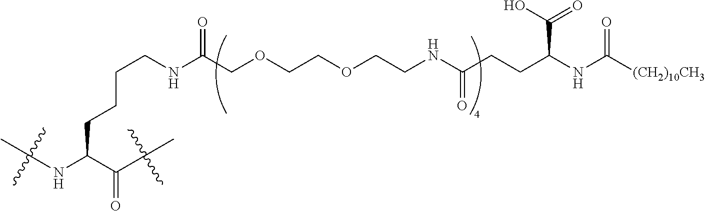

K(PEG2PEG2PEG2PEG2gEC12)

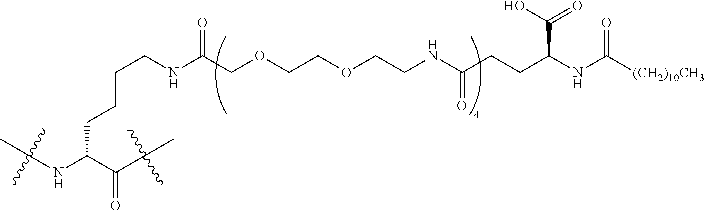

k(PEG2PEG2PEG2PEG2gEC12)

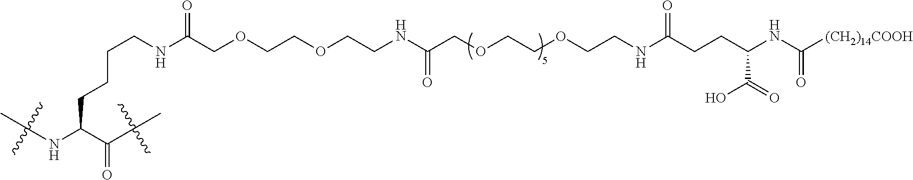

K(PEG2PEG6gEC16OH)

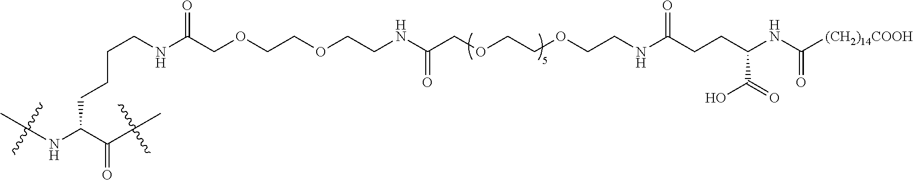

k(PEG2PEG6gEC16OH)

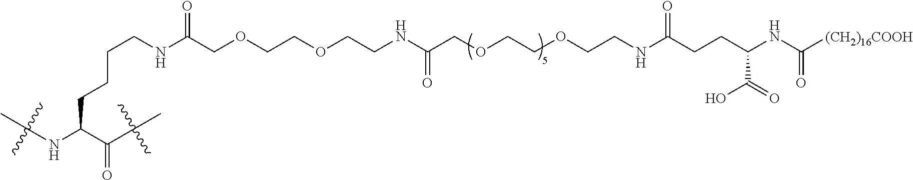

K(PEG2PEG6gEC18OH)

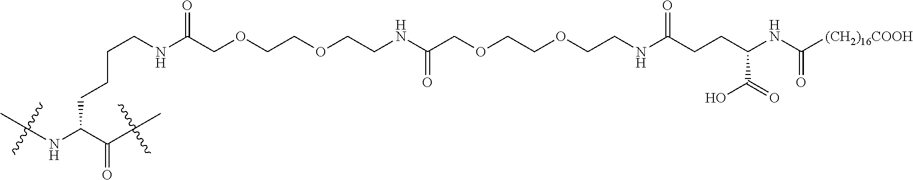

k(PEG2PEG6gEC18OH)

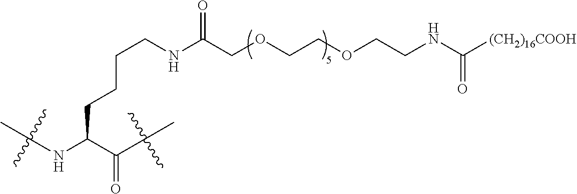

K(PEG6C18OH)

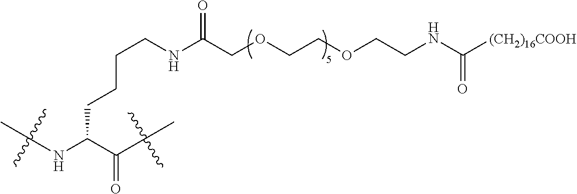

k(PEG6C18OH)

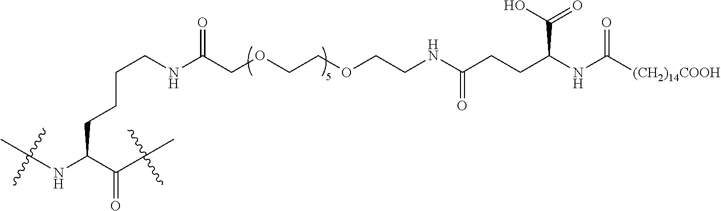

K(PEG6gEC16OH)

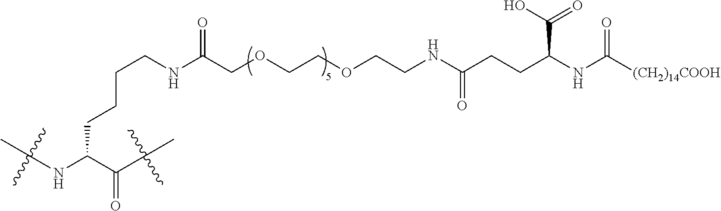

k(PEG6gEC16OH)

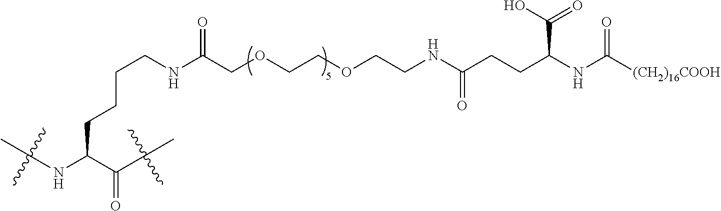

K(PEG6gEC18OH)

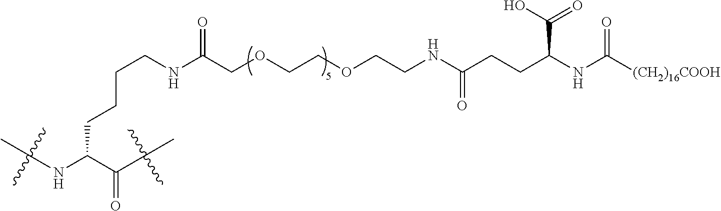

k(PEG6gEC18OH)

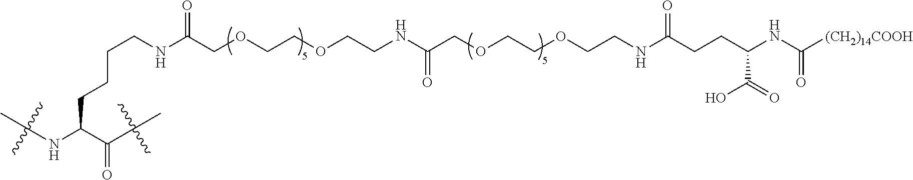

K(PEG6PEG6gEC16OH)

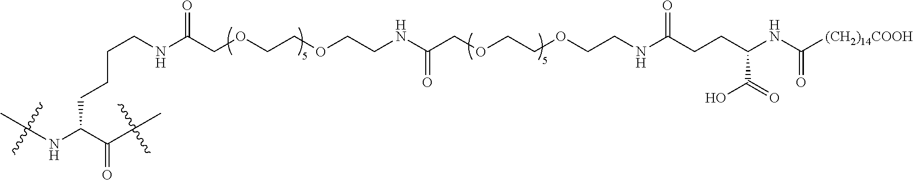

k(PEG6PEG6gEC16OH)

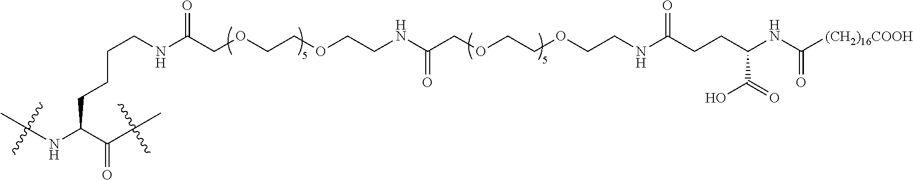

K(PEG6PEG6gEC18OH)

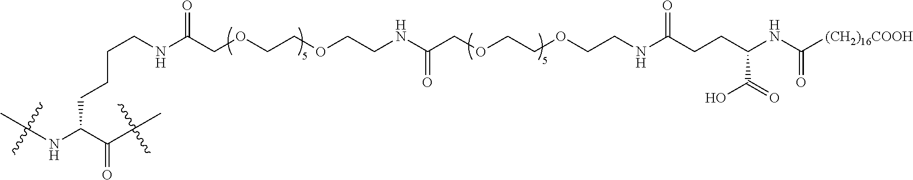

k(PEG6PEG6gEC18OH)

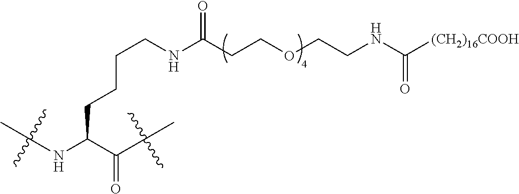

K(Peg4_C18_Diacid)

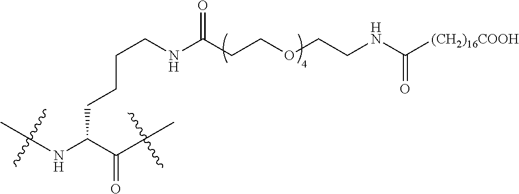

k(Peg4_C18_Diacid)

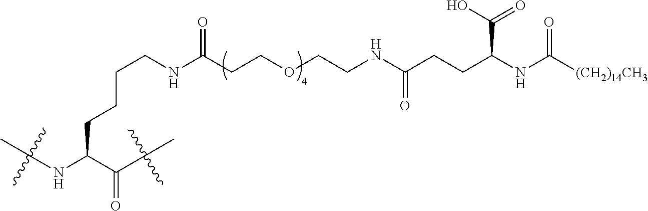

K(Peg4_IsoGlu_Palm)

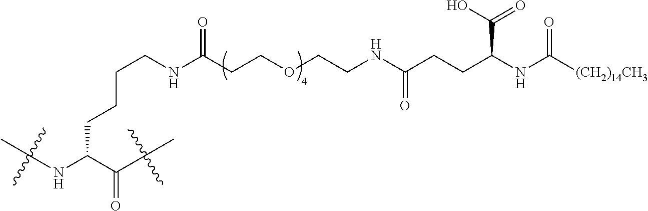

k(Peg4_IsoGlu_Palm)

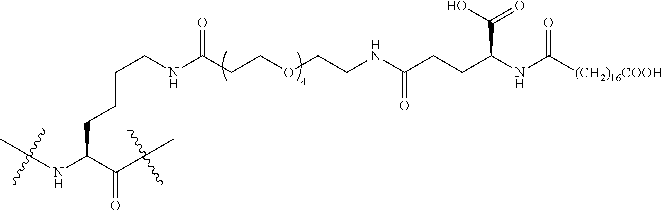

K(Peg4_IsoGlu_C18_Diacid)

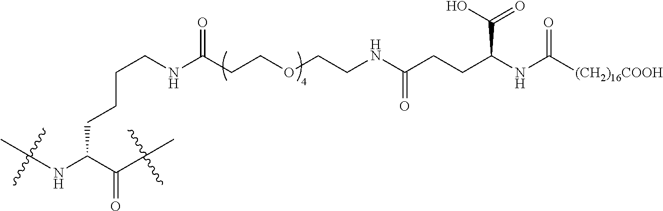

k(Peg4_IsoGlu_C18_Diacid)

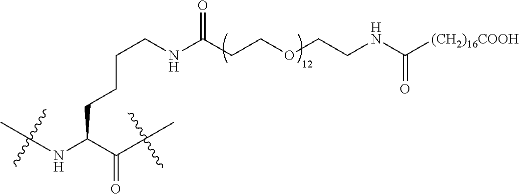

K(PEG12C18OH), K(PEG12_C18_Diacid)

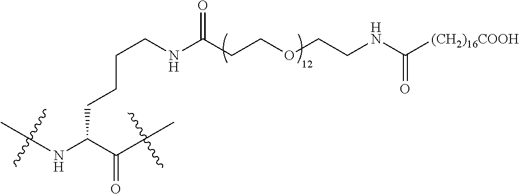

k(PEG12C18OH), k(PEG12_C18_Diacid)

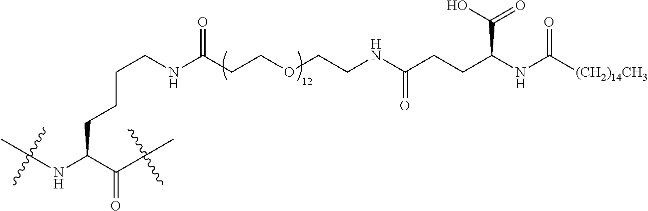

K(PEG12gEC16), K(PEG12_IsoGlu_Palm)

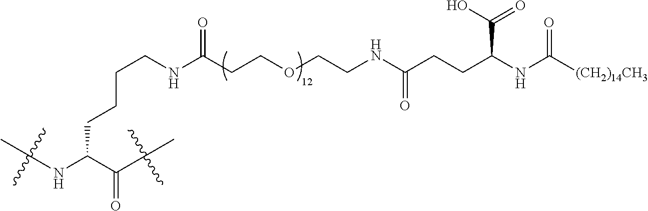

k(PEG12gEC16), k(PEG12_IsoGlu_Palm)

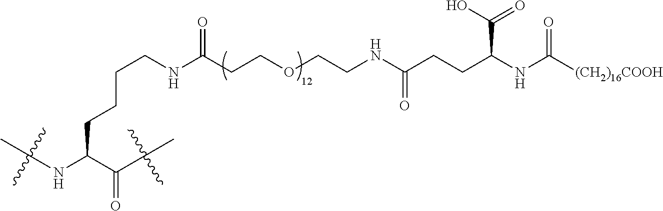

K(PEG12gEC18OH), K(PEG12_IsoGlu_C18_Diacid)

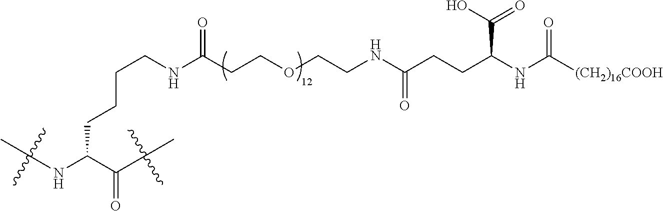

k(PEG12gEC18OH), k(PEG12_IsoGlu_C18_Diacid)

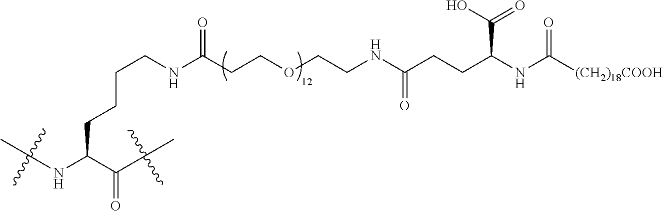

K(PEG12gEC20OH)

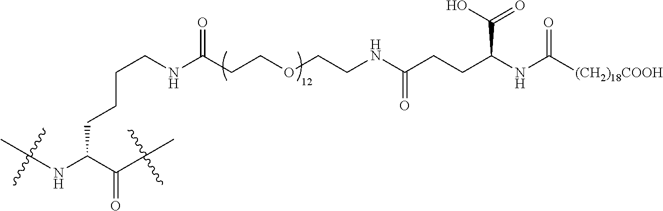

k(PEG12gEC20OH)

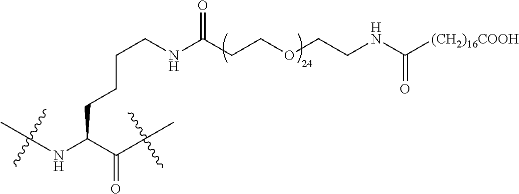

K(PEG24-C18OH)

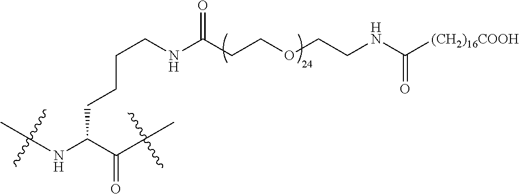

k(PEG24-C18OH)

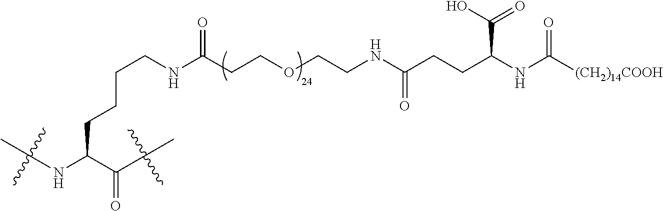

K(PEG24-gEC16OH)

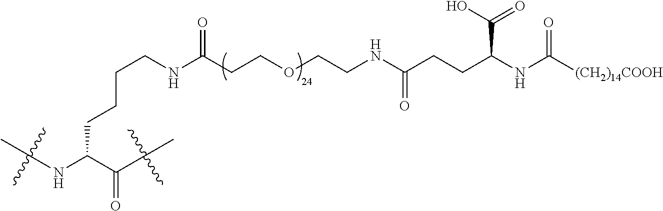

k(PEG24-gEC16OH)

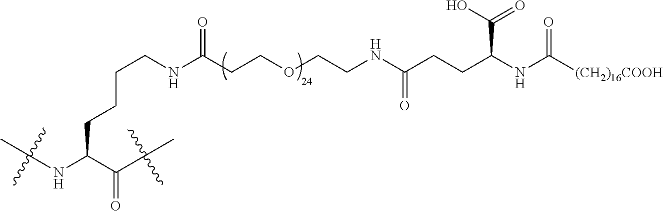

K(PEG24-gEC18OH)

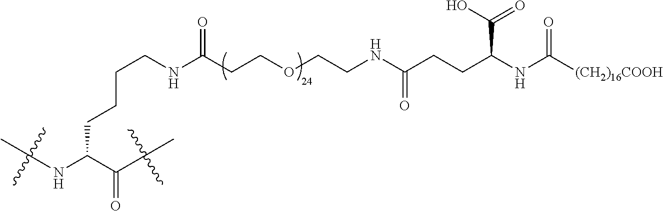

k(PEG24-gEC18OH)

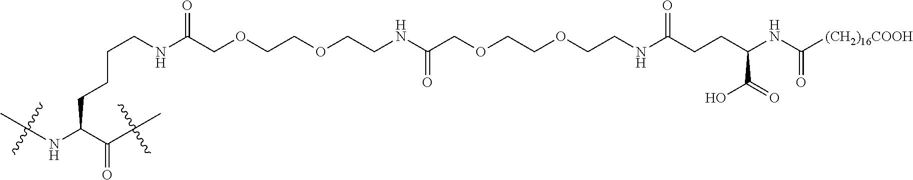

K(PEG2PEG2-DgEC18OH)

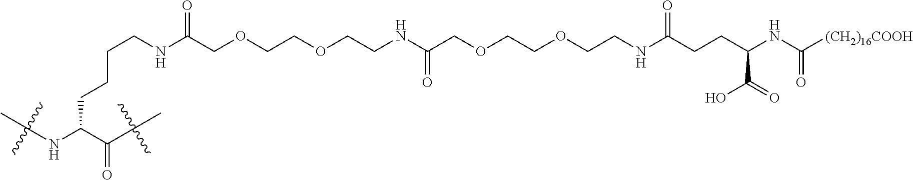

k(PEG2PEG2-DgEC18OH)

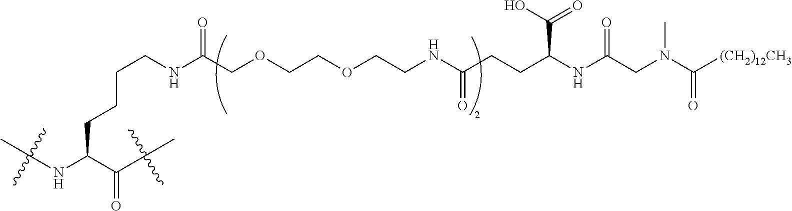

K(PEG2PEG2gESarC14)

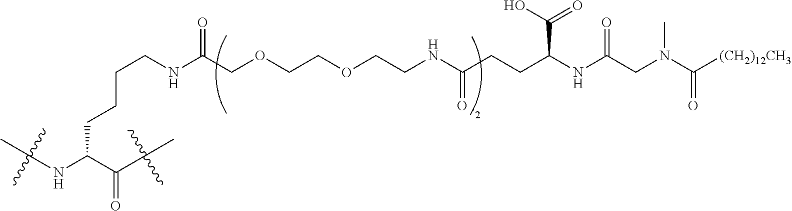

k(PEG2PEG2gESarC14)

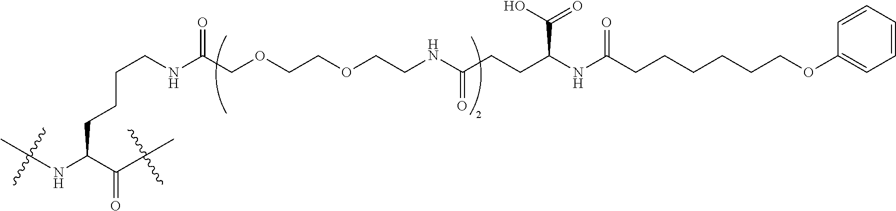

K(PEG2PEG2gEC6X)

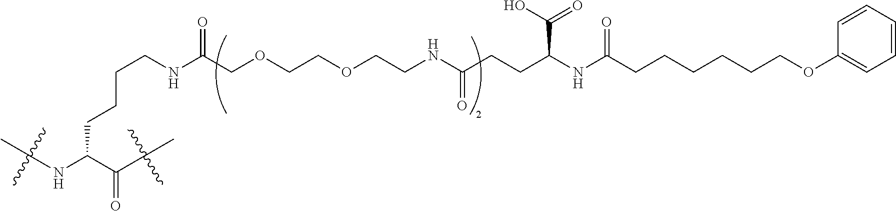

k(PEG2PEG2gEC6X)

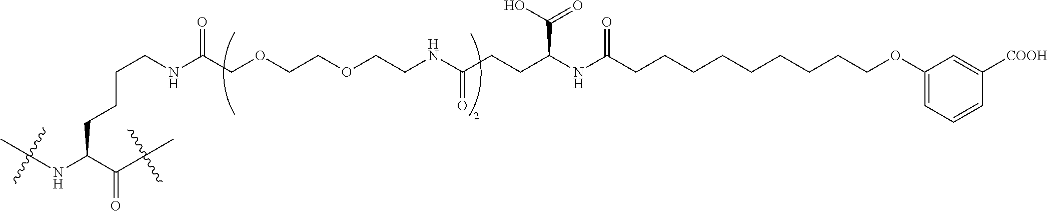

K(PEG2PEG2gEmXOH)

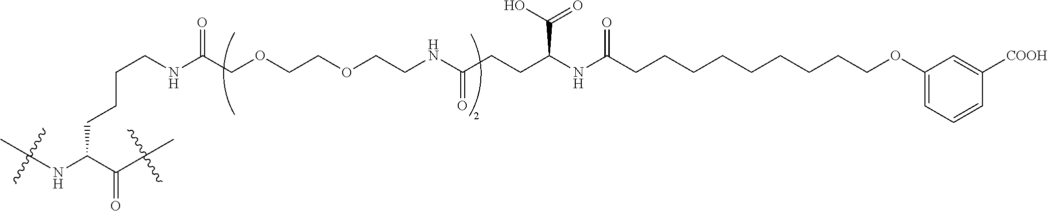

k(PEG2PEG2gEmXOH)

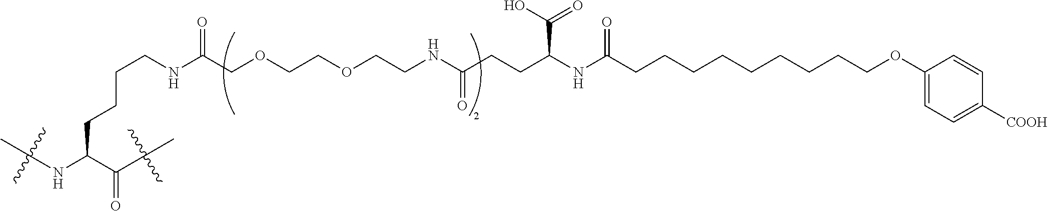

K(PEG2PEG2gEpXOH)

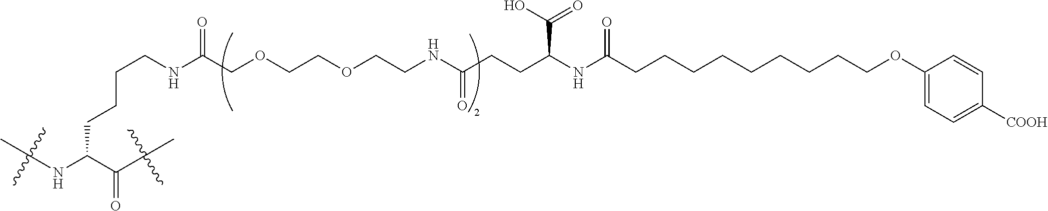

k(PEG2PEG2gEpXOH)

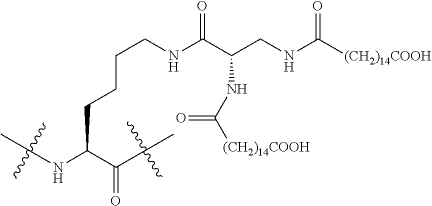

K(DAP(C16OH)2)

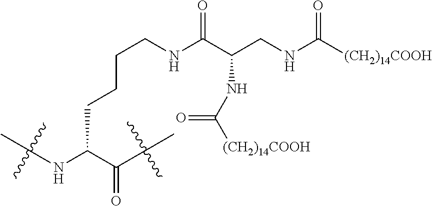

k(DAP(C16OH)2)

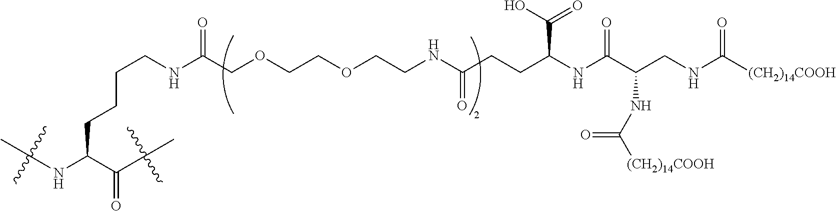

K(PEG2PEG2gEDAP(C16OH)2)

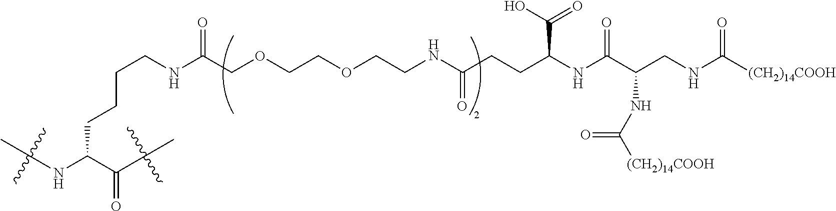

k(PEG2PEG2gEDAP(C16OH)2)

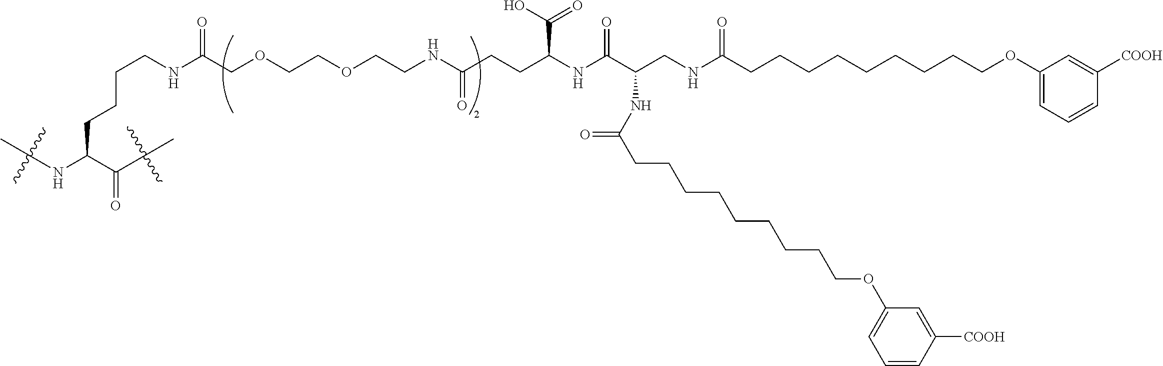

K[PEG2PEG2-gEDAP-(mXOH)2]

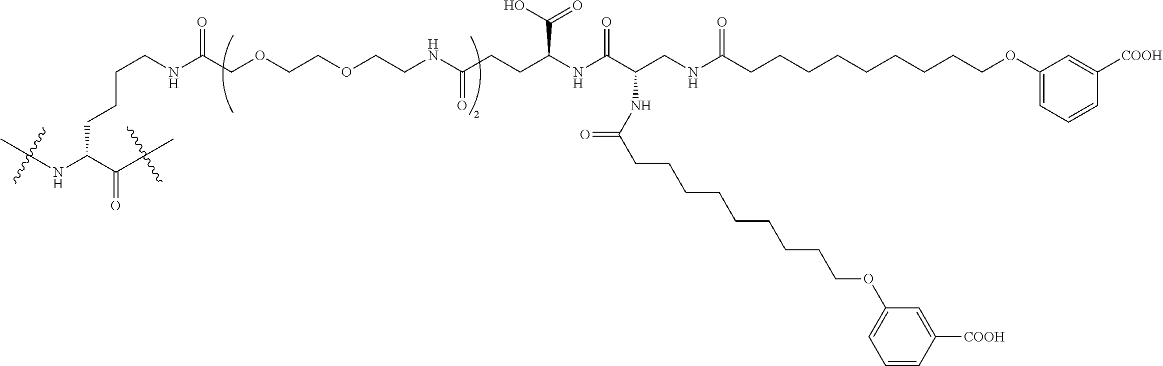

k[PEG2PEG2-gEDAP-(mXOH)2]

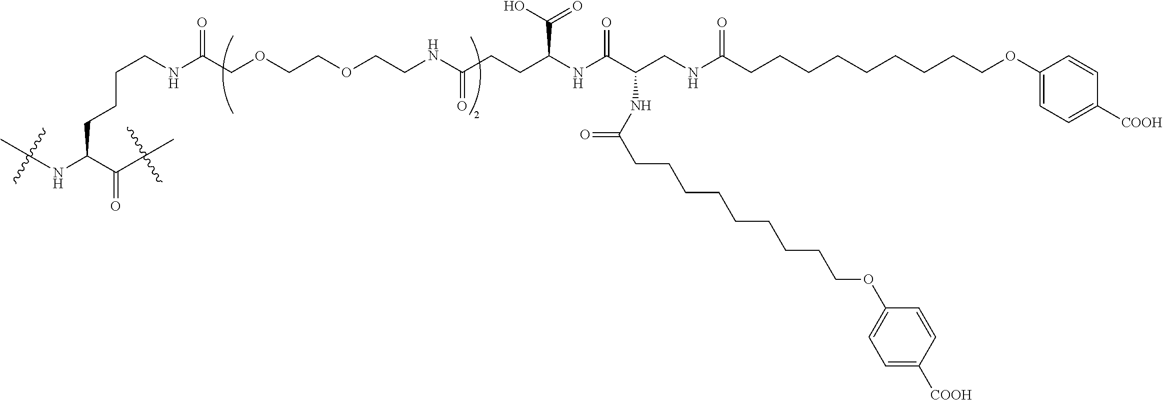

K[PEG2PEG2-gEDAP(pXOH)2]

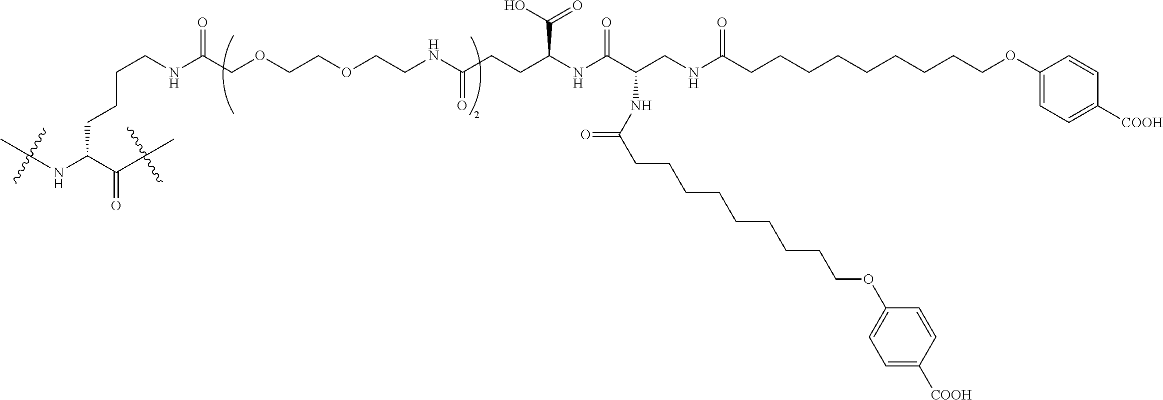

k[PEG2PEG2-gEDAP(pXOH)2]

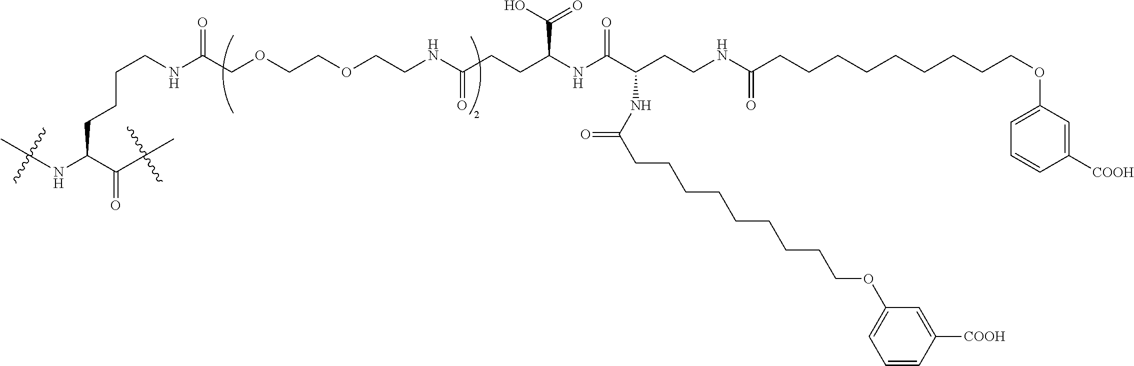

K(PEG2PEG2gEDab(mXOH)2)

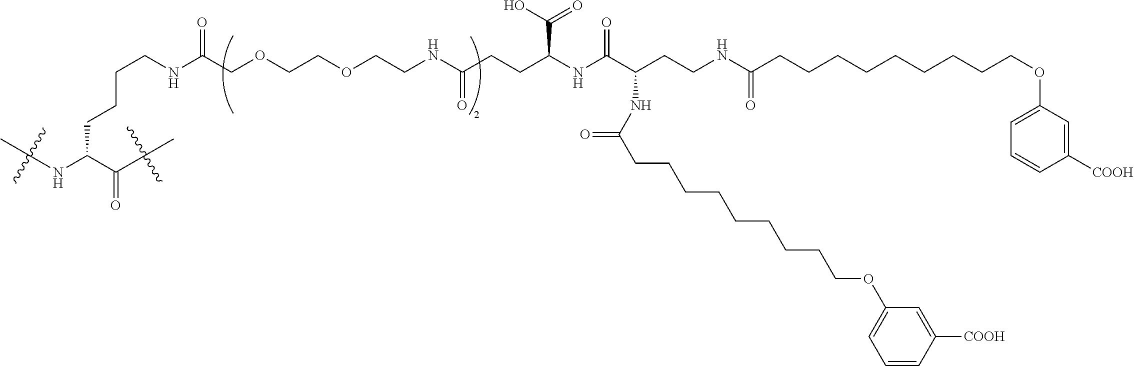

k(PEG2PEG2gEDab(mXOH)2)

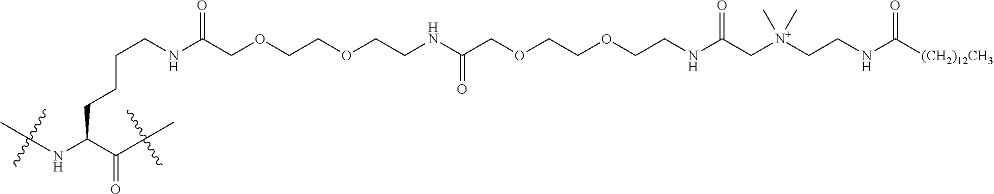

K(PEG2PEG2SP6C14)

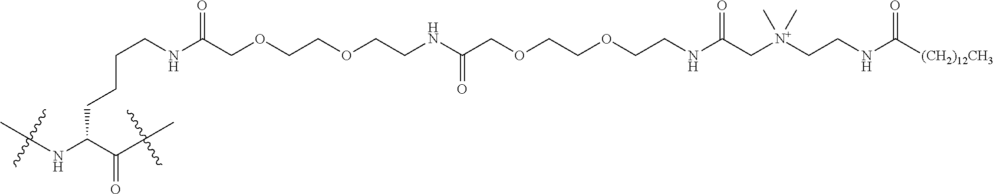

k(PEG2PEG2SP6C14)

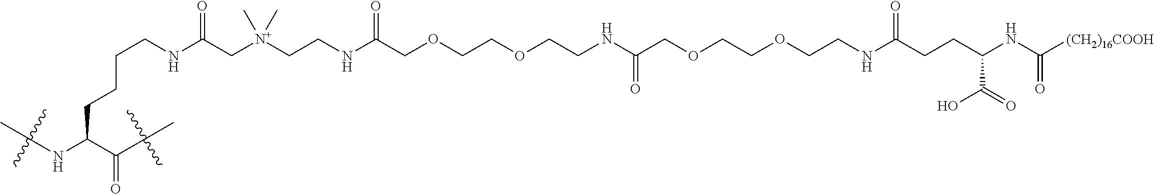

K(Sp6PEG2PEG2gEC18OH)

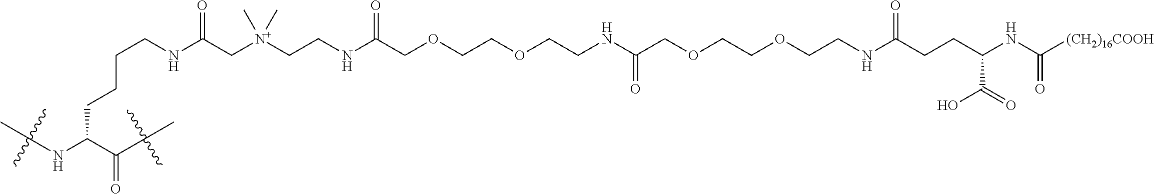

k(Sp6PEG2PEG2gEC18OH)

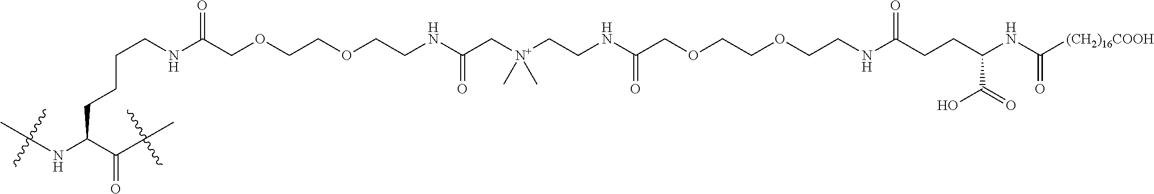

K(PEG2Sp6PEG2gEC18OH)

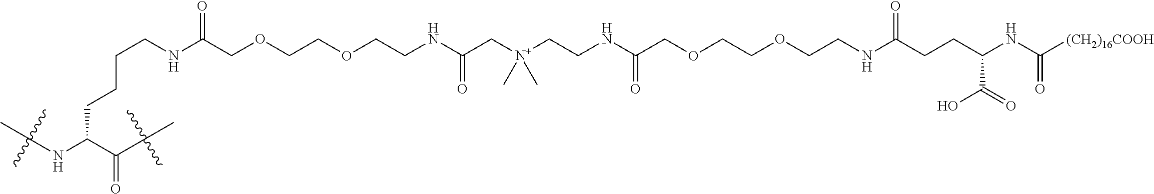

k(PEG2Sp6PEG2gEC18OH)

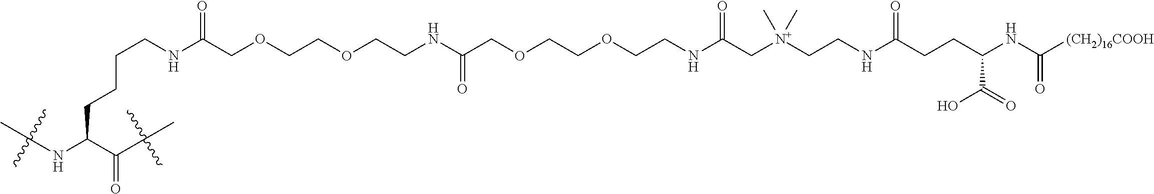

K(PEG2PEG2SP6gEC18OH)

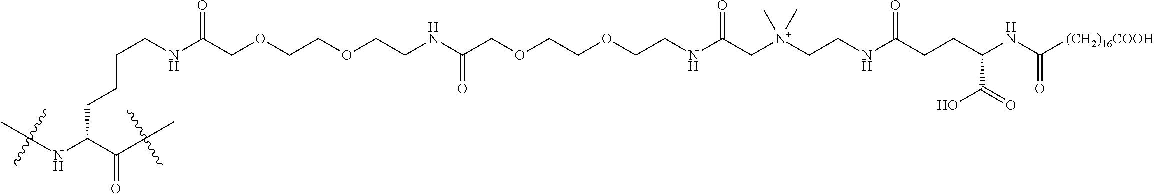

k(PEG2PEG2SP6gEC18OH)

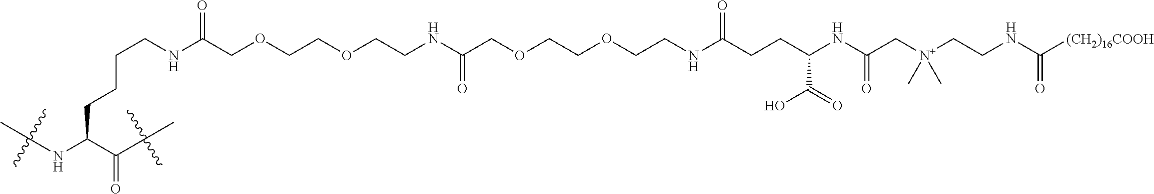

K(PEG2PEG2gESp6C18OH)

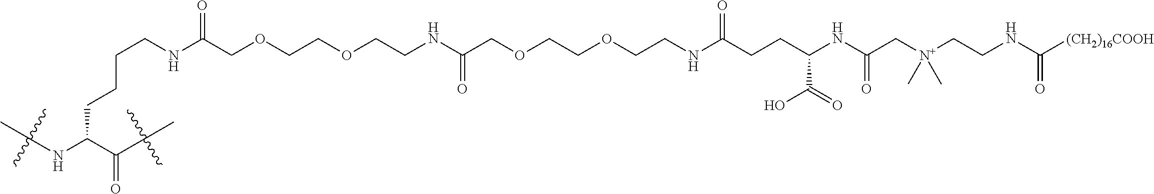

k(PEG2PEG2gESp6C18OH)

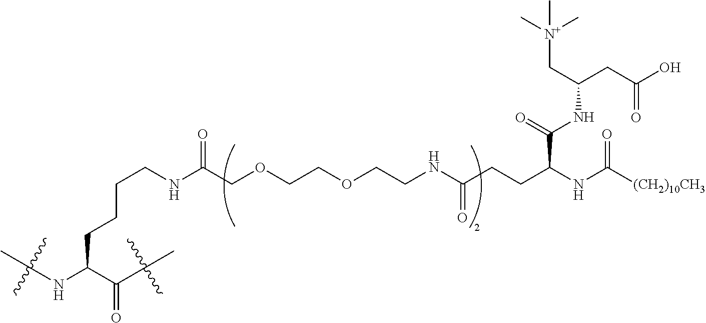

K(PEG2PEG2gE(C)C12)

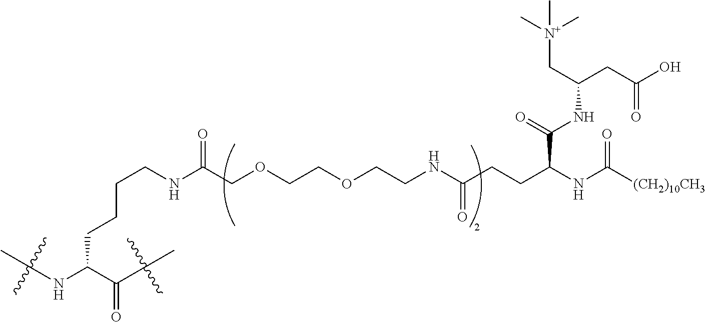

k(PEG2PEG2gE(C)C12)

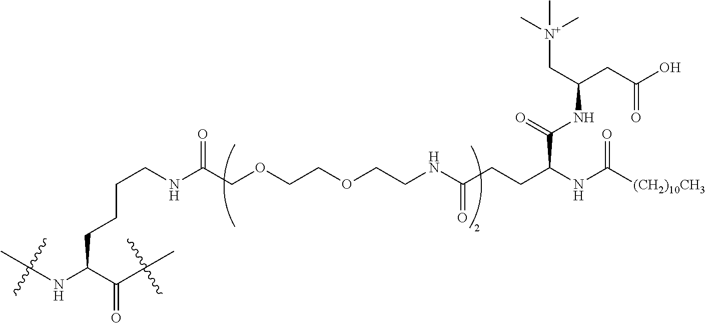

K(PEG2PEG2gE(c)C12)

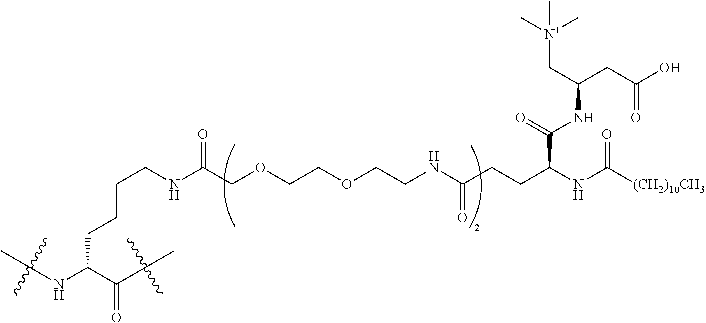

k(PEG2PEG2gE(c)C12)

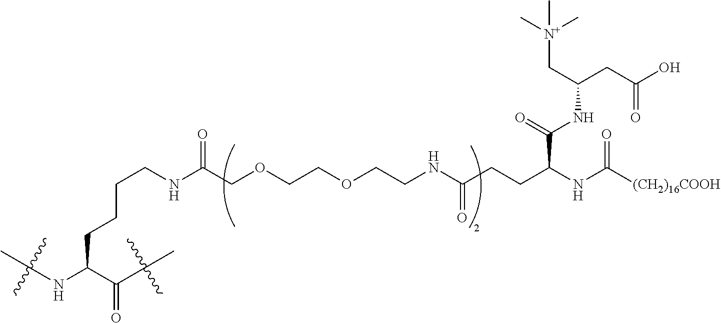

K(PEG2PEG2gE(C)C18OH)

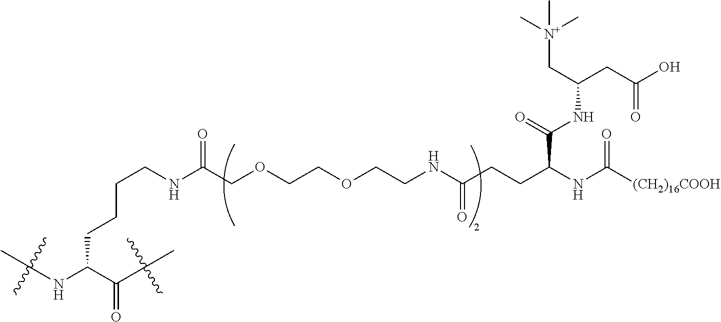

k(PEG2PEG2gE(C)C18OH)

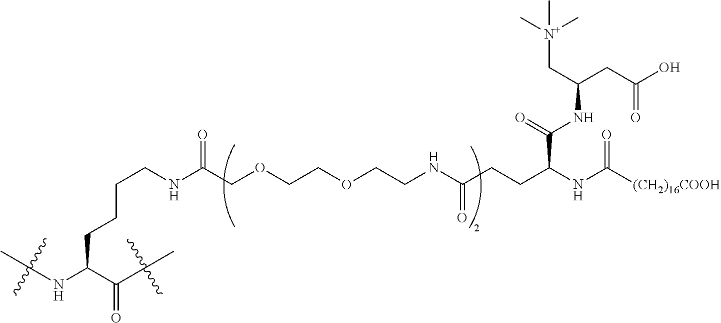

K(PEG2PEG2gE(c)C18OH)

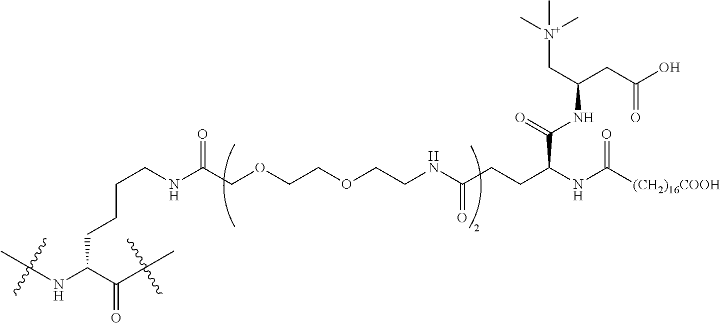

k(PEG2PEG2gE(c)C18OH)

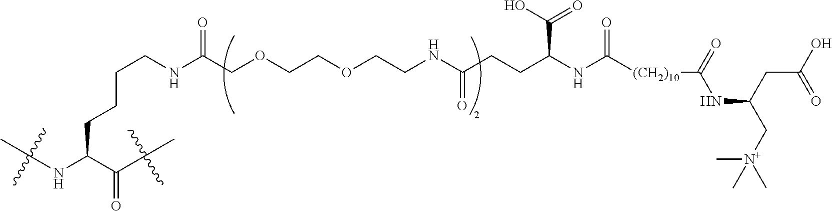

K(PEG2PEG2gEC12OH(C))

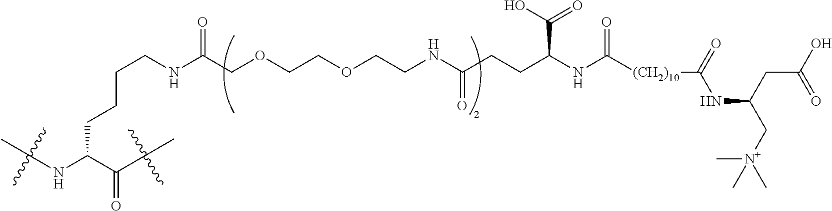

k(PEG2PEG2gEC12OH(C))

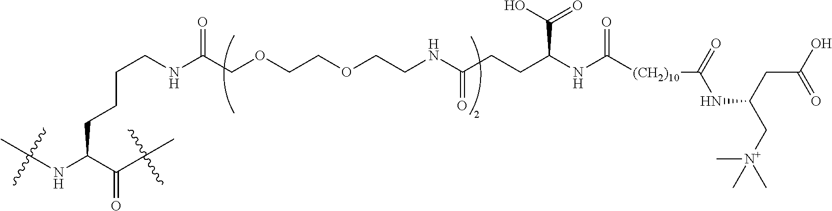

K(PEG2PEG2gEC12OH(c))

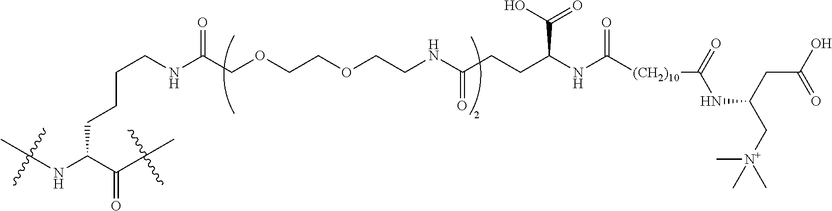

k(PEG2PEG2gEC12OH(c))

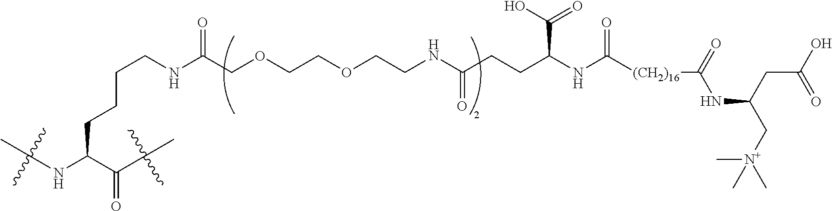

K(PEG2PEG2gEC18OH(C))

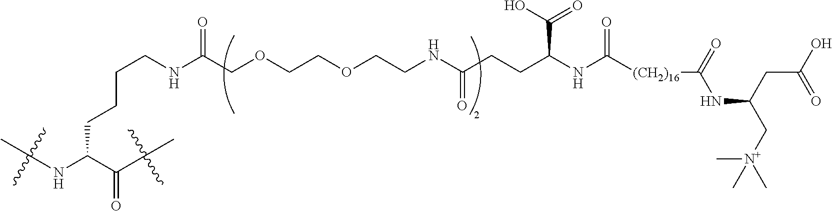

k(PEG2PEG2gEC18OH(C))

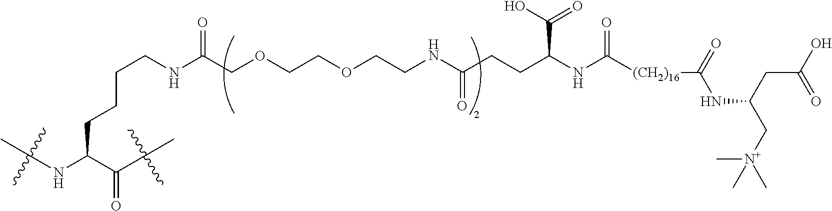

K(PEG2PEG2gEC18OH(c))

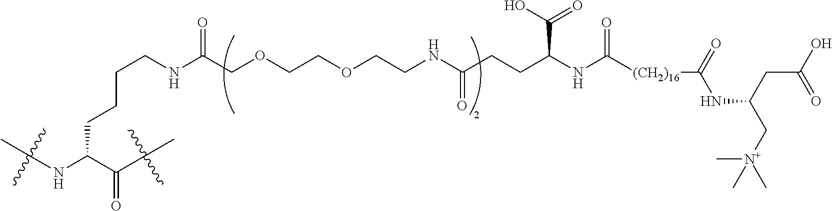

k(PEG2PEG2gEC18OH(c))

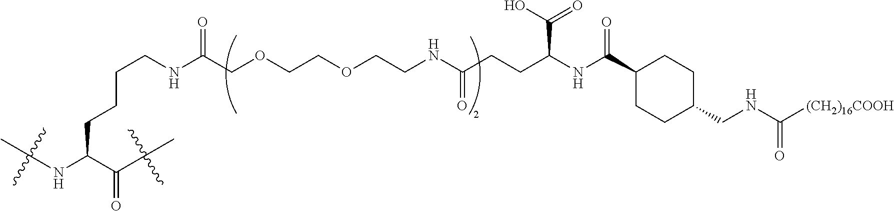

K(PEG2PEG2gETrxC18OH)

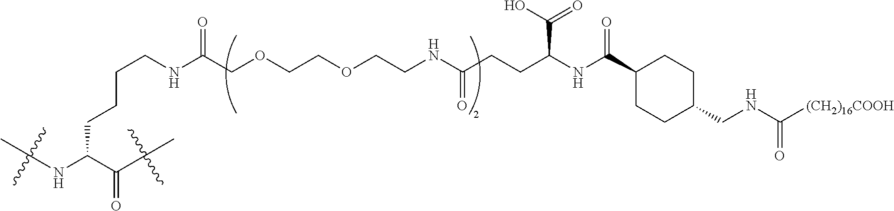

k(PEG2PEG2gETrxC18OH)

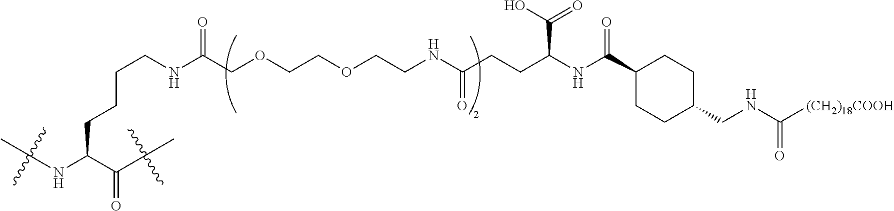

K(PEG2PEG2-gETrxC20OH)

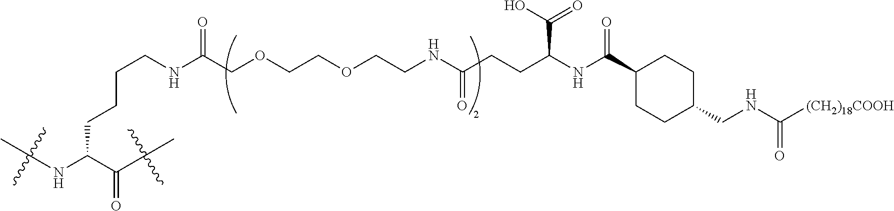

k(PEG2PEG2-gETrxC20OH)

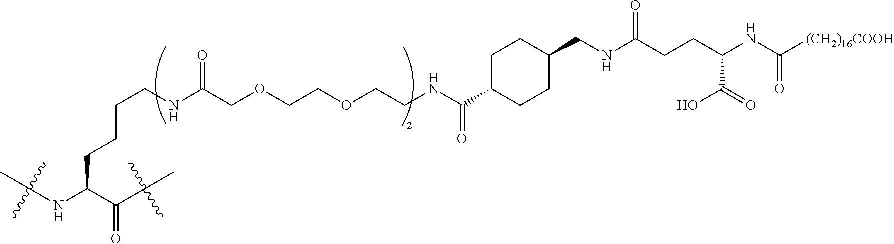

K(PEG2PEG2-TrxgEC18OH)

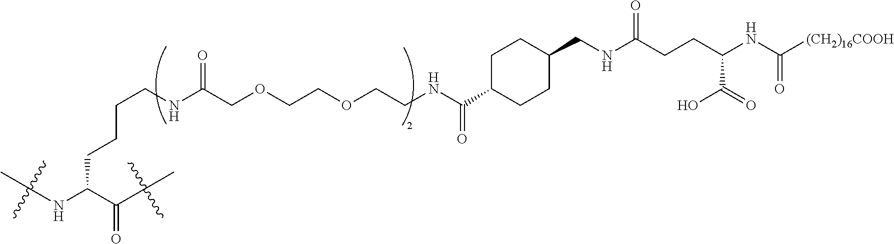

k(PEG2PEG2-TrxgEC18OH)

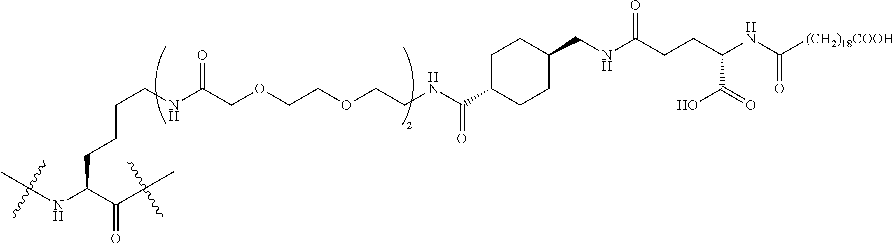

K(PEG2PEG2-TrxgEC20OH)

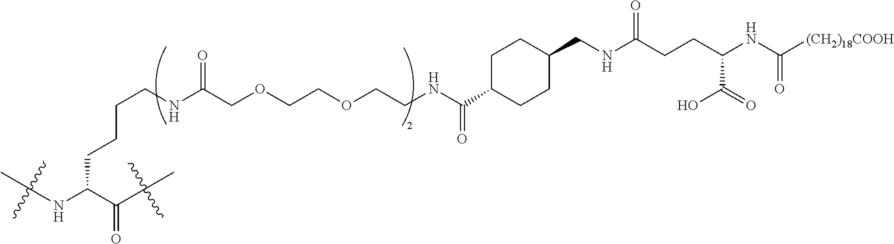

k(PEG2PEG2-TrxgEC20OH)

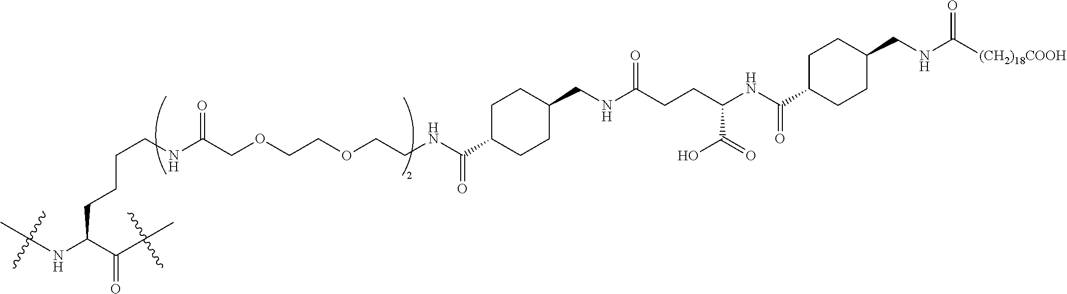

K(PEG2PEG2-TrxgETrxC20OH)

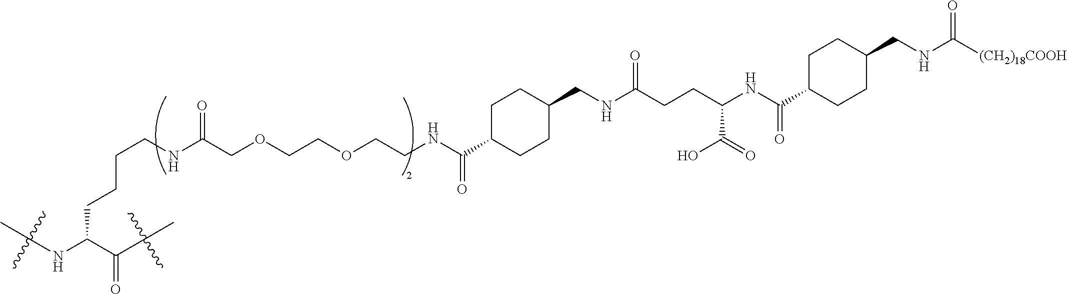

k(PEG2PEG2-TrxgETrxC20OH)

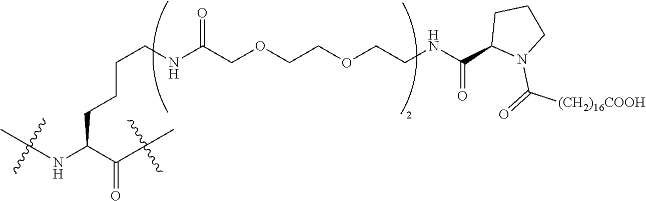

K(PEG2PEG2-pC18OH)

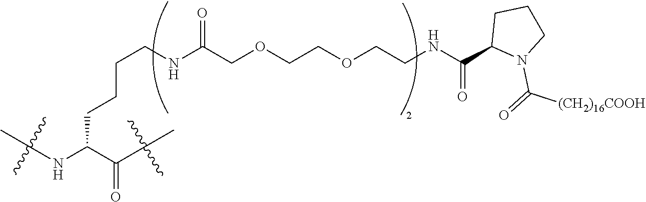

k(PEG2PEG2-pC18OH)

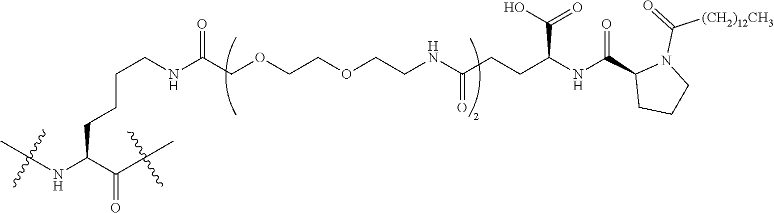

K(PEG2PEG2gEProC14)

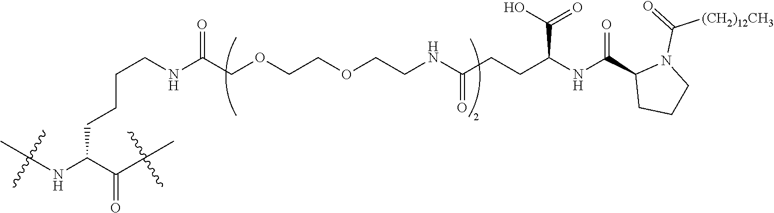

k(PEG2PEG2gEProC14)

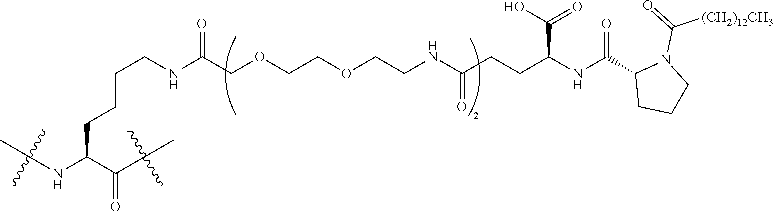

K(PEG2PEG2gEDProC14)

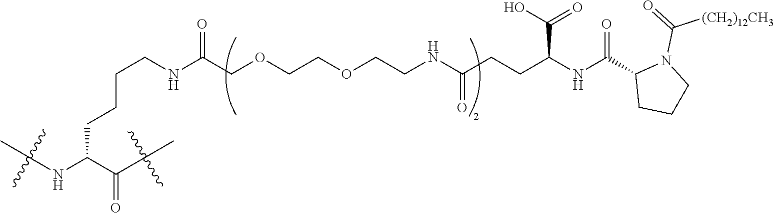

k(PEG2PEG2gEDProC14)

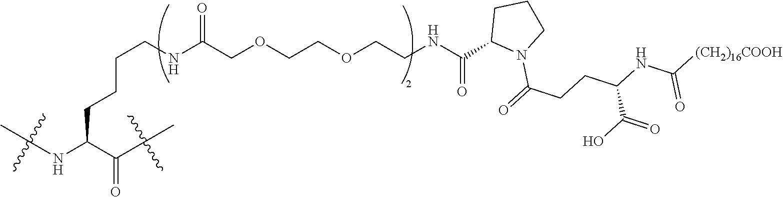

K(PEG2PEG2PgEC18OH)

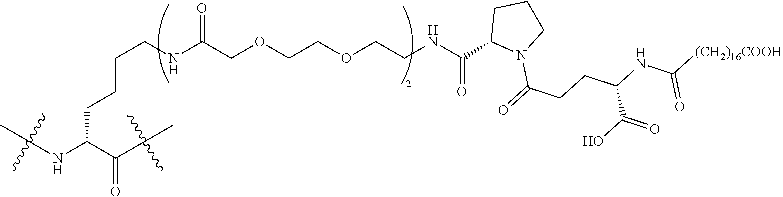

k(PEG2PEG2PgEC18OH)

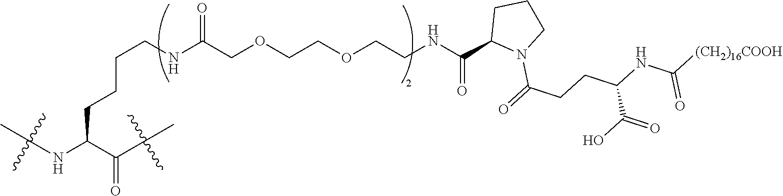

K(PEG2PEG2pgEC18OH)

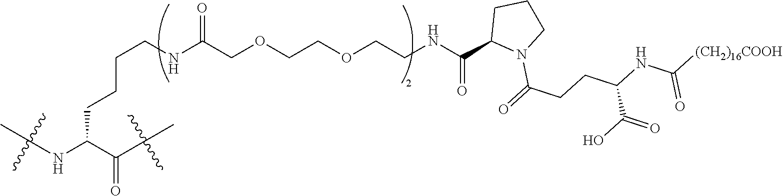

k(PEG2PEG2pgEC18OH)

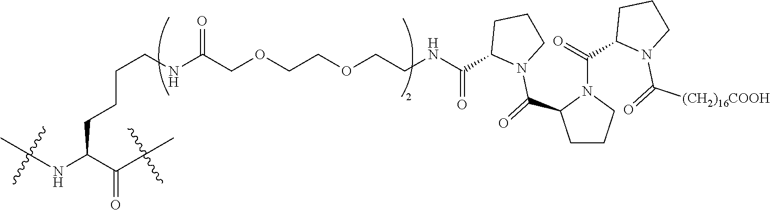

K(PEG2PEG2-PPPC18OH)

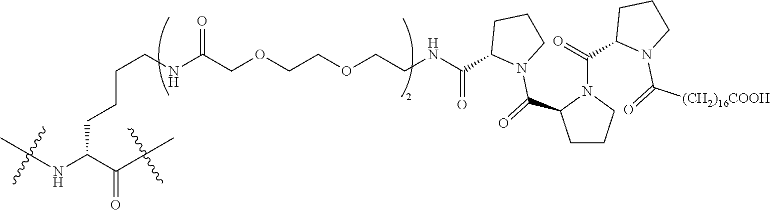

k(PEG2PEG2-PPPC18OH)

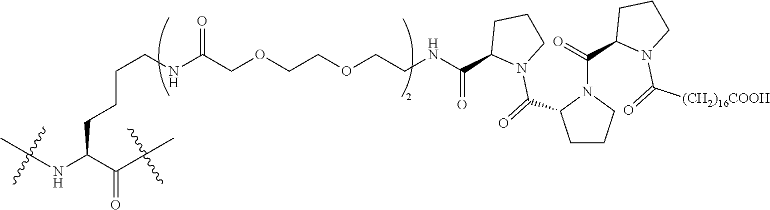

K(PEG2PEG2-pppC18OH)

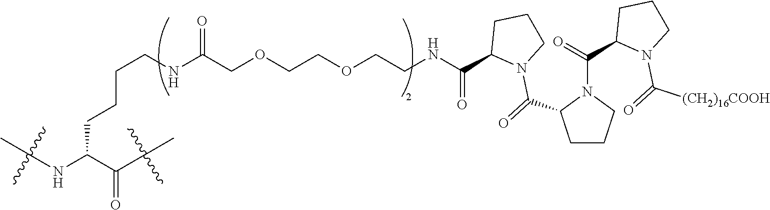

k(PEG2PEG2-pppC18OH)

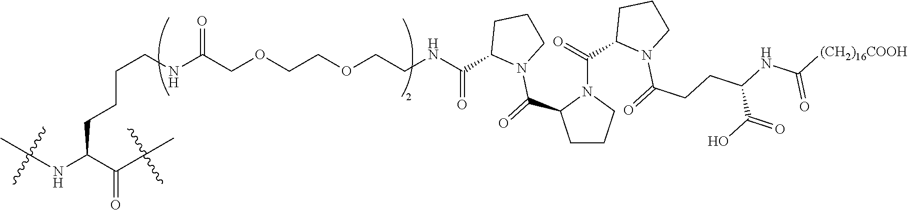

K(PEG2PEG2PPPgEC18OH)

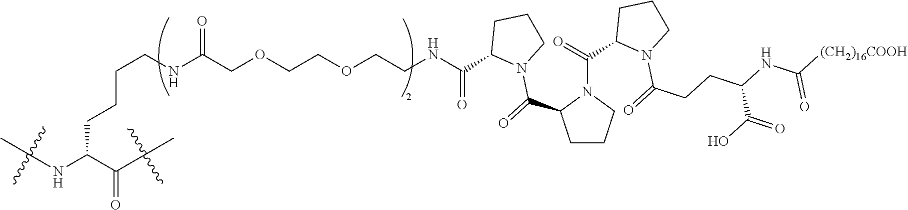

k(PEG2PEG2PPPgEC18OH)

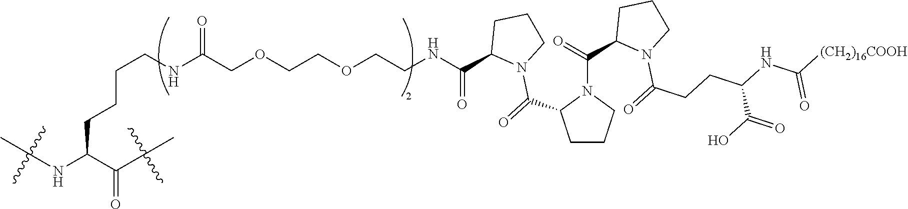

K(PEG2PEG2pppgEC18OH)

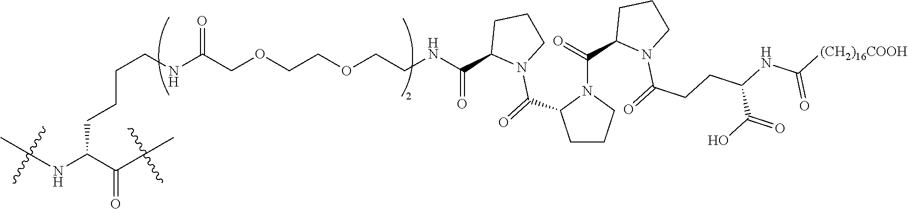

k(PEG2PEG2pppgEC18OH)

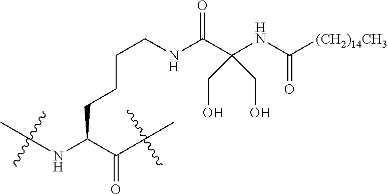

K(GolAC16)

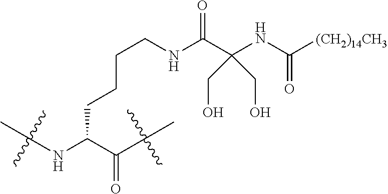

k(GolAC16)

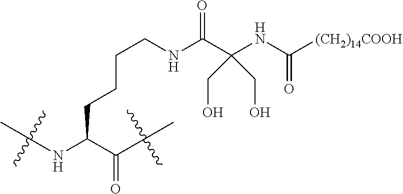

K(GolAC16OH)

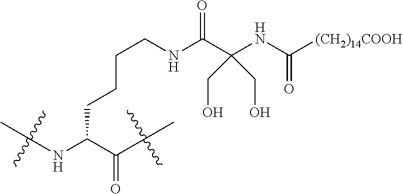

k(GolAC16OH)

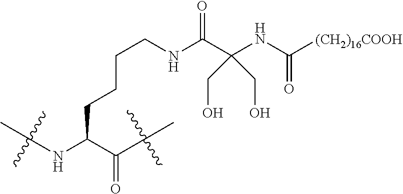

K(GolAC18OH)

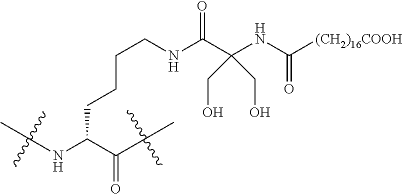

k(GolAC18OH)

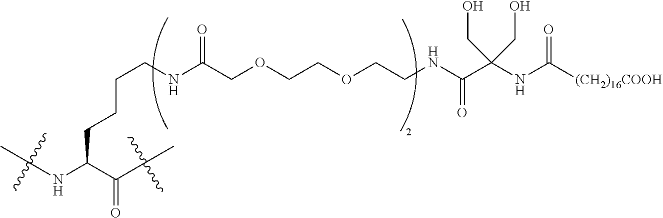

K(PEG2PEG2 GolAC18OH)

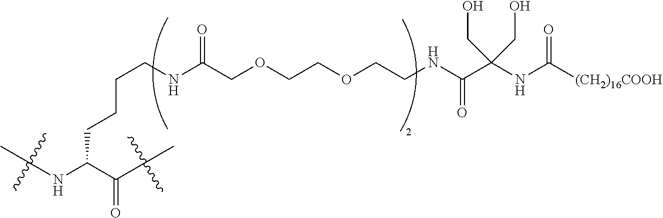

k(PEG2PEG2 GolAC18OH)

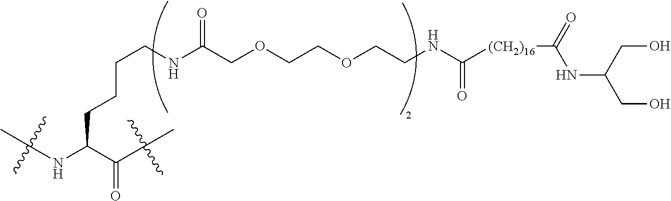

K(PEG2PEG2-C18GolB)

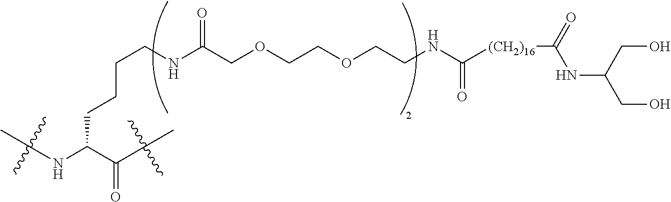

k(PEG2PEG2-C18GolB)

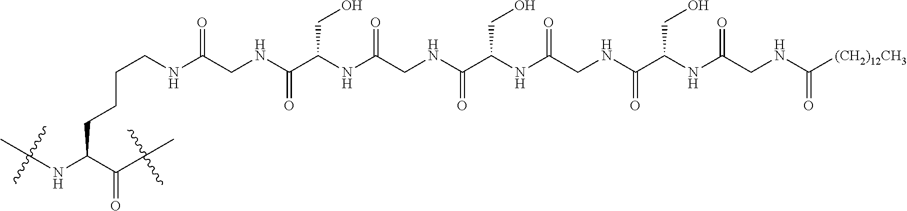

K(GSGSGSGC14)

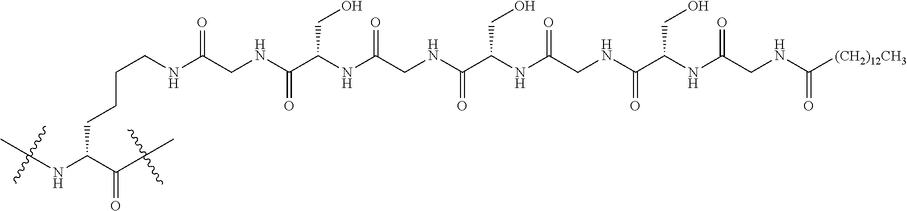

K(GSGSGSGC14)

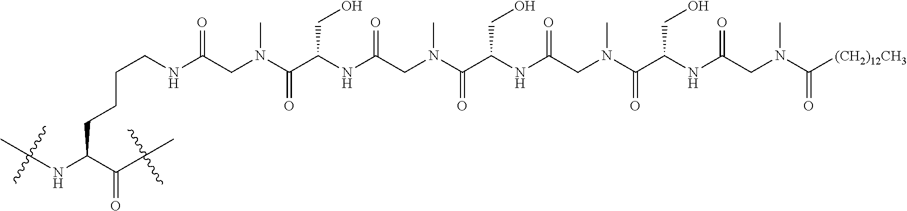

K(Sar-S-Sar-S-Sar-S-Sar-C14)

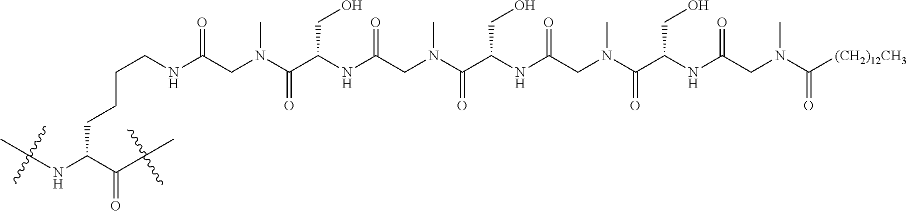

K(Sar-S-Sar-S-Sar-S-Sar-C14)

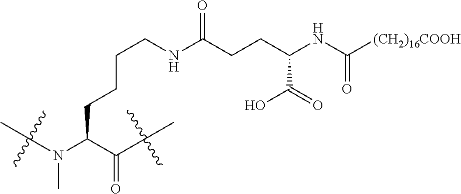

NMeK(gEC18OH)

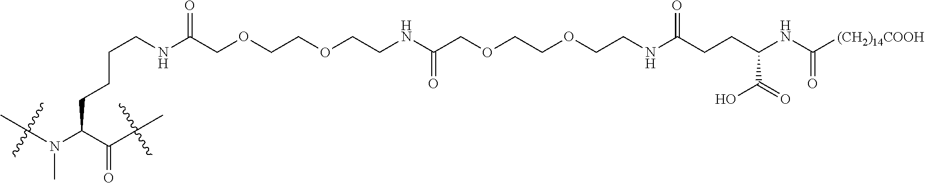

NMeK(PEG2PEG2gEC16OH), K(1PEG21PEG2IsoGluC16Diacid)

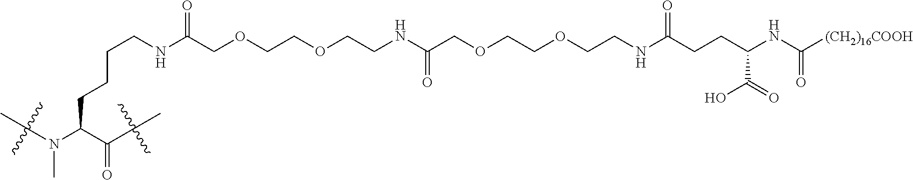

NMeK(PEG2PEG2gEC18OH), K(1PEG21PEG2IsoGluC18Diacid),

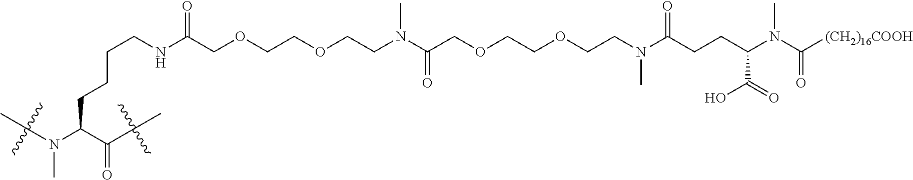

NMeK(PEG2NMePEG2NMegENMeC18OH)

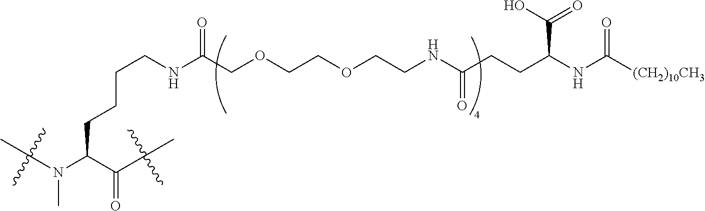

NMeK(PEG2PEG2PEG2PEG2gEC12)

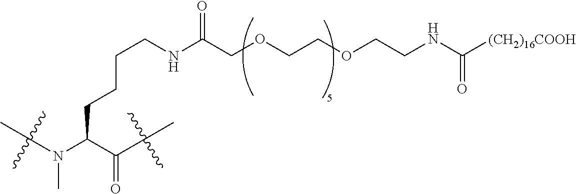

NMeK(PEG6C18OH)

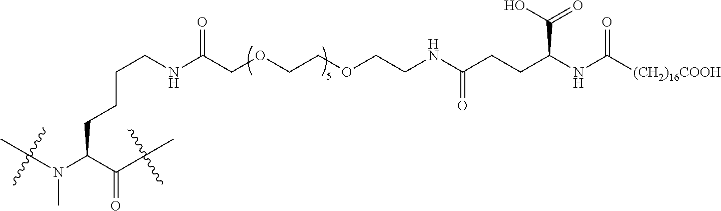

NMeK(PEG6gEC18OH)

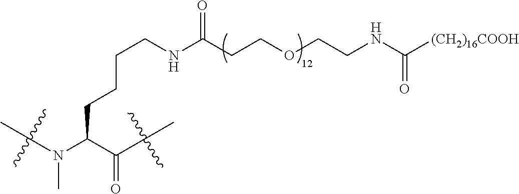

NMeK(PEG12C18OH)

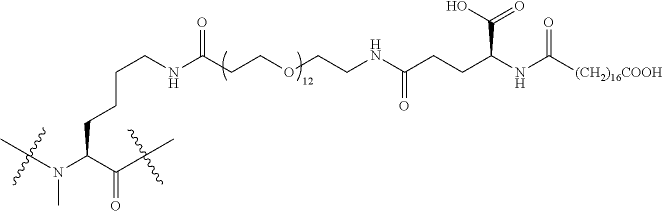

NMeK(PEG12gEC18OH)

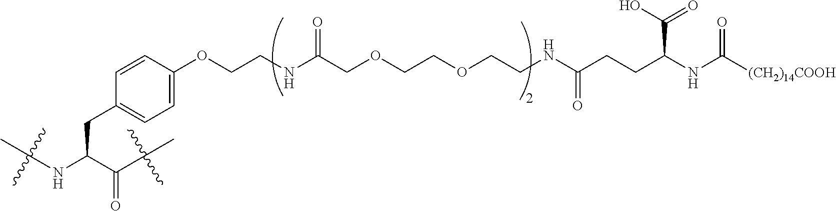

AEF(PEG2PEG2gEC16OH)

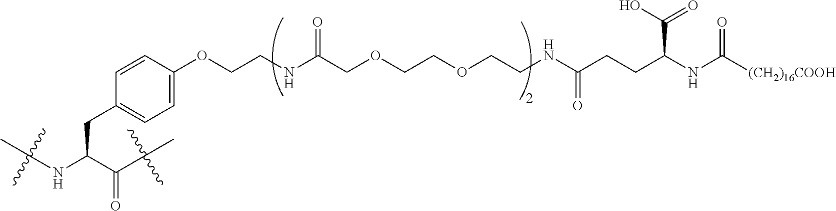

AEF(PEG2PEG2gEC18OH)

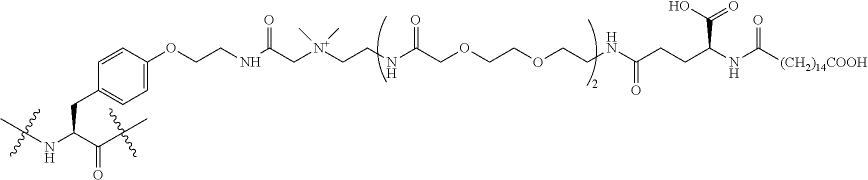

AEF(SP6PEG2PEG2gEC16OH)

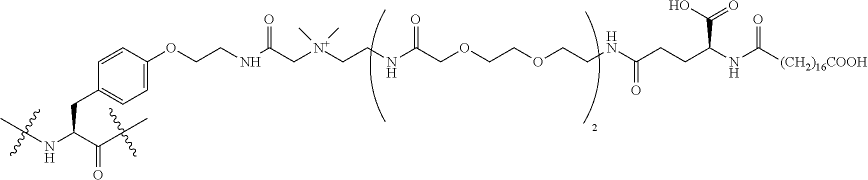

AEF(SP6PEG2PEG2gEC18OH)

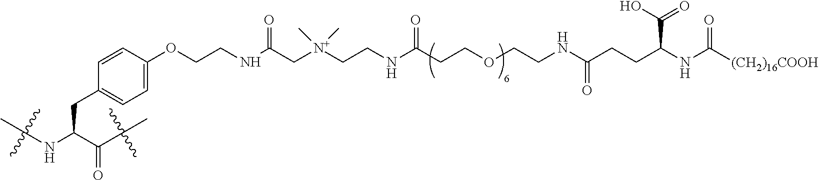

AEF(SP6PEG6gEC18OH)

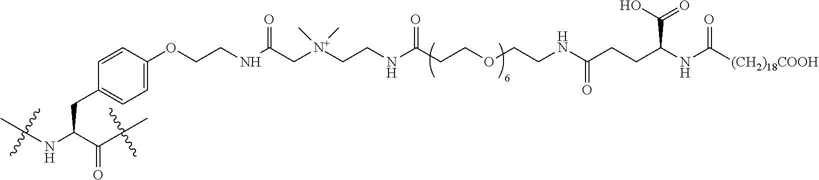

AEF(SP6PEG6gEC20OH)

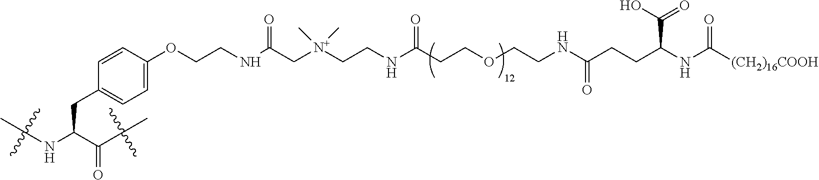

AEF(SP6PEG12gEC18OH)

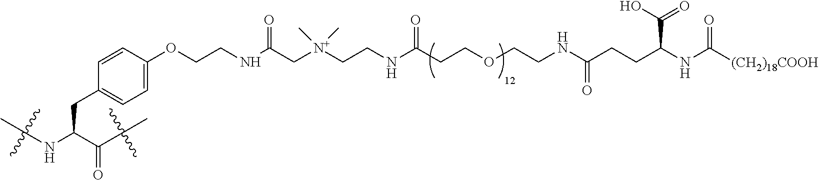

AEF(SP6PEG12gEC20OH)

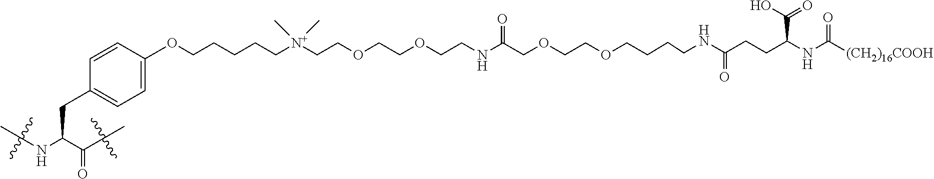

TMAPF(PEG2PEG2gEC18OH)

### Example 1: General Procedure for Solid-Phase Synthesis of Peptides

Peptide were chemically synthesized using optimized 9-fluorenylmethoxy carbonyl (Fmoc) solid phase peptide synthesis protocols. For C-terminal amides, Rink-amide MBHA resin was used. The side chain protecting groups were as follows: Lys(iv-Dde): iv-Dde; d-Arg: Pbf, NMe-Lys: iv-Dde; Thr, Glu: O-tBlutyl; Asn, Pen: Trityl; AEF: Boc. For coupling, a two to five-fold excess of a solution containing Fmoc amino acid, HATU and DIEA (1:0.95:2) in DMF was added to swelled resin for 1 to 16 hours. Double coupling is employed when coupling 2Nal and 3Pya. Fmoc protecting group removal was achieved by treatment with a DMF, piperidine (4:1) solution for 30 min. iv-dde protecting group on NMeLys or Lys removal was achieved by treatment with 3% hydrazine hydrate in DMF solution for 20 min*3 times. The cycles are repeated until the full-length peptide is obtained.

Certain materials and reagents are listed below.

| | | Coupling | Monitored | Reaction | |
|---|---|---|---|---|---|
| # | Materials | reagents | method | time | Note |
| 1 | Fmoc-NMeLys(iv- | HATU (1.90 eq) | ninhydrin color | 2 h | |
| | dde)-OH (2.0 eq) | and DIEA (4.0 eq) | reaction. | | |
| 2 | Fmoc-Lys(iv-dde)-OH | HATU (1.90 eq) | ninhydrin color | 2 h | |
| | (2.0 eq) | and DIEA (4.0 eq) | reaction. | | |
| 3 | Fmoc-Sar-OH (3.0 eq) | HATU (2.85 eq) | ninhydrin color | 1 h | |
| | | and DIEA (6.0 eq) | reaction. | | |
| 4 | Fmoc-3Pya-OH | HATU (1.90 eq) | tetrachlor color | 2 | Double |
| | (2.0 eq) | and DIEA (4.0 eq) | test | h*2 | coupling |
| 5 | Fmoc-Asn(Trt)-OH | HATU (2.85 eq) | ninhydrin color | 2 h | |
| | (3.0 eq) | and DIEA (6.0 eq) | reaction. | | |
| 6 | Fmoc-Glu(OtBu)-OH | HATU (2.85 eq) | ninhydrin color | 1 h | |
| | (3.0 eq) | and DIEA (6.0 eq) | reaction. | | |
| 7 | Fmoc-THP-OH (2.0 | HATU (1.90 eq) | ninhydrin color | 2 h | |
| | eq) | and DIEA (4.0 eq) | reaction. | | |
| 8 | Fmoc-2Nal-OH (2.0 | HATU (1.90 eq) | tetrachlor color | 2 | Double |
| | eq) | and DIEA (4.0 eq) | test | h*2 | coupling |
| 9 | Fmoc-AEF(Boc)-OH | HATU (1.90 eq) | ninhydrin color | 1 h | |
| | (2.0 eq) | and DIEA (4.0 eq) | reaction. | | |
| 10 | Fmoc-Pen(Trt)-OH | HATU (2.85 eq) | ninhydrin color | 2 h | |
| | (3.0 eq) | and DIEA (6.0 eq) | reaction. | | |
| 11 | Fmoc- K(Ac)-OH (2.0 | HATU (1.90 eq) | ninhydrin color | 1 h | |
| | eq) | and DIEA (4.0 eq) | reaction. | | |
| 12 | Fmoc-7MeTrp-OH | HATU (1.9 eq) | ninhydrin color | 2 h | |
| | (2.0 eq) | and DIEA (4.0 eq) | reaction. | | |
| 13 | Fmoc-Thr(tBu)-OH | HATU (2.85 eq) | ninhydrin color | 1 h | |
| | (3.0 eq) | and DIEA (6.0 eq) | reaction. | | |
| 14 | Fmoc-Asn(Trt)-OH | HATU (2.85 eq) | ninhydrin color | 2 h | |
| | (3.0 eq) | and DIEA (6.0 eq) | reaction. | | |
| 15 | Fmoc-Pen(Trt)-OH | HATU (2.85 eq) | ninhydrin color | 2 h | |
| | (3.0 eq) | and DIEA (6.0 eq) | reaction. | | |
| 16 | Fmoc-d-Arg(Pbf)-OH | HATU (2.85 eq) | ninhydrin color | 1 h | |
| | (3.0 eq) | and DIEA (6.0 eq) | reaction. | | |
| 17 | 10% Ac₂O/5% NMM/ | N/A | ninhydrin color | 1 h | |
| | 85% DMF | | reaction. | | |
| — | 3% hydrazine hydrate | N/A | ninhydrin color | 20 | De-iv- |
| | in DMF | | reaction. | min*3times | dde(Note3) |
| 18 | PEG2NMePEG2NMegENMeC18OH | DIC (2.0 eq) and | ninhydrin color | 16 h | |
| | (2.0 eq) | HOAT (2.0 eq) | reaction | | |
| 19 | PEG2NMePEG2NMegENMeC18Tetrazole | DIC (2.0 eq) and | ninhydrin | 16 h | 16 |
| | (2.0 eq) | HOAT (2.0 eq) | color reaction | | |

### General Procedure for Cleavage of Peptides Off Resin

Side chain deprotection and cleavage of the peptides was achieved by stirring the dry resin in a solution of trifluoroacetic acid, water, DTT and tri-isopropylsilane (90:2.5:5:2.5) for 3 hours. The mixture was then filtered and cold methyl tert-butyl ether (MTBE) was added to the combined filtrate to precipitate the peptide. The resulting mixture was centrifuged (3000 rpm, 3 min) and decanted. The pellet was washed with MTBE and centrifuged. The pellet was lyophilized to provide the linear peptide.

### General Procedure for Cyclization

To effect cyclization of thiol-containing residues, iodine solution in MeOH (0.1M) was added to a solution of the linear peptide (20% MeCN/H2O (1 mmol/L)) drop-wise until a yellow color persisted. After about 2 h, analysis by LCMS showed that the linear peptide was no longer present. The excess iodine was quenched by the addition of 1M Na₂S₂O₃ in water (turned colorless instantly).

### General Procedure for Purification of Peptides

Purification of the peptides was achieved using reverse-phase high performance liquid chromatography (RP-HPLC). Purification of the cyclized peptides was achieved using preparative RP-HPLC with a C18 column with a flow rate of 20-250 mL/min. Separation was achieved using gradients of buffer B in A (Buffer A: 0.075% TFA in water; Buffer B: ACN). (Note 1). Analysis was performed using a C18 column with a flow rate of 1 mL/min (Note 2).

### Note 1: Preparative HPLC Methods

Prep. HPLC Method A: Description: Mobile Phase: 0.075% TFA in water (solvent A) and acetonitrile (solvent B) Column: YMC-Actus Triart C18, 250*30 mm, 5 μm, 120 Å column; Flow Rate: 20 mL/min; Wavelength: UV 220 nm&254 nm; Oven Temp. Room temperature

### Note 2: Analytical HPLC Method:

Mobile Phase: 0.1% TFA in water (solvent A) and 0.1% TFA in acetonitrile (solvent B), using the elution gradient 10%-80% (solvent B) over 0.9 minutes and using the elution gradient 80%-90% for 0.6 minutes at a flow rate of 1.0 ml/min; Column: Xbridge C18, 3.5 um, 2.1*30 mm; Wavelength: UV 220 nm&254 nm; Column temperature: 30° C.; MS ionization: ESI

### Example 2: Synthesis of SEQ ID NO: 80

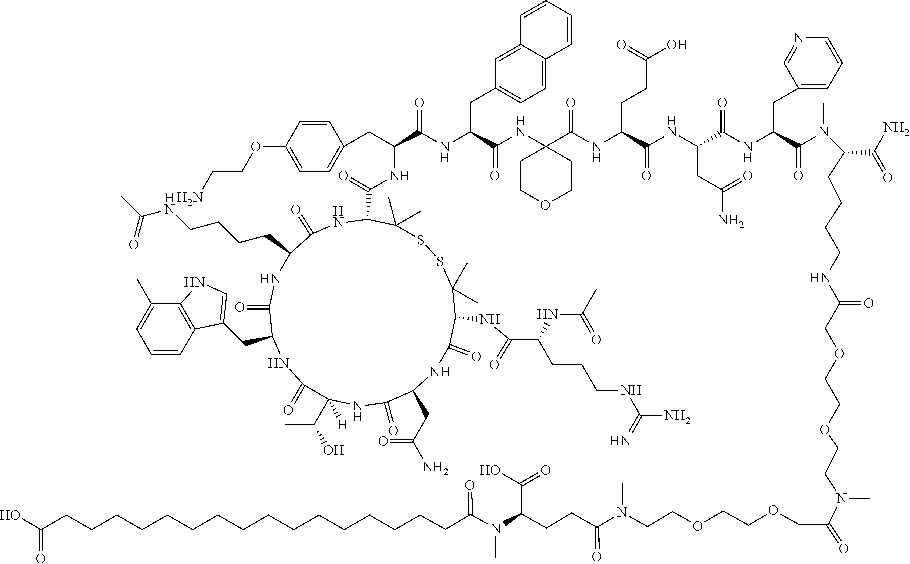 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)[C@@H](CCCCNC(=O)COCCOCCN(C)C(=O)COCCOCCN(C)C(=O)CC[C@H](C(=O)O)N(C)C(=O)CCCCCCCCCCCCCCCCC(=O)O)C(N)=O)CCOCC2)NC1=O]

The peptide was synthesized using standard Fmoc chemistry:
1) DMF and MBHA Resin (0.30 mmol, 1.1 g, sub: 0.26 mmol/g) were combined in a vessel, and the resin was allowed to swell for two hours.
2) A solution of 20% piperidine/DMF was added and the suspension was mixed for 30 min.
3) The resin was then drained and washed with DMF for 30 seconds*5 times.
4) Fmoc-amino acid solution was then added and mixed with the resin for 30 seconds before addition of a solution of HATU and DIEA in DMF. The reaction was allowed to proceed under nitrogen for 1-4 hours.
5) A solution of 20% piperidine/DMF was added and the suspension was mixed for 30 min.
6) Steps 2 to 5 were repeated for subsequent amino acid couplings. The coupling reactions were monitored by ninhydrin or tetrachlor color test, and upon completion, the resin was washed with DMF 5 times. Once peptide synthesis was complete, the resin was washed with MeOH 3 times and dried by vacuum.

### Monitoring Method:

1. Ninhydrin test: A: 5% ninhydrin/EtOH; B: 80% phenol/EtOH; C: pyridine
2. Tetrachlor color test: A: 2% tetrachlor/DMF; B: 2% aldehyde/DMF 110° C. for 3 min
   Synthetic Method for Removal of ivDde Protecting Group:

Deprotection of ivDde on NMeLys: The resin was washed with 50 mL of DMF (5×0.1 min) followed by addition of 3% hydrazine hydrate in DMF (50 mL) for 20 min*3 times (ninhydrin color reaction), and washed with DMF 5 times.

### Peptide Cleavage:

1) To the flask containing the side chain protected peptide at room temperature was added 30 mL cleavage buffer (5.0% DTT/2.5% H₂O/2.5% TIS/90% TFA), and mixture was stirred for 3 hrs.
2) The mixture was filtered and washed with 5 mL TFA. The combined filtrate was triturated with cold methyl tertbutyl ether (MTBE). The mixture was centrifuged (3000 rpm, 3 min) and decanted. The pellet was washed with MTBE and centrifuged.
3) The residue was lyophilized to give intermediate 1 (620 mg, 71.6% yield, crude)

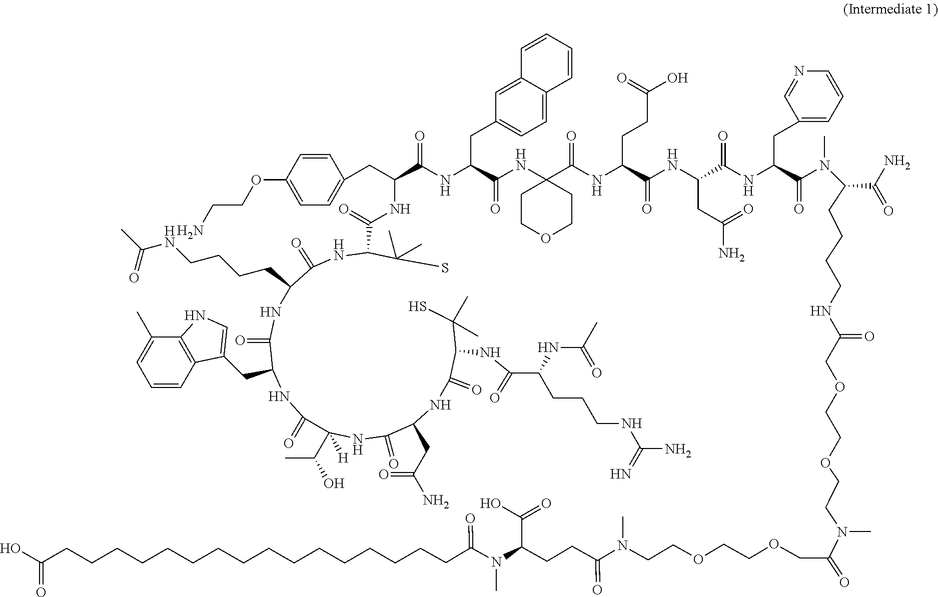 [SMILES: CC(=O)NCCCC[C@H](NC(=O)[C@H](Cc1c[nH]c2c(C)cccc12)NC(=O)[C@@H](NC(=O)[C@H](CC(N)=O)NC(=O)[C@@H](NC(=O)[C@@H](CCCNC(=N)N)NC(C)=O)C(C)(C)S)[C@@H](C)O)C(=O)N[C@H](C(=O)N[C@@H](Cc1ccc(OCCN)cc1)C(=O)N[C@@H](Cc1ccc2ccccc2c1)C(=O)NC1(C(=O)N[C@@H](CCC(=O)O)C(=O)N[C@@H](CC(N)=O)C(=O)N[C@@H](Cc2cccnc2)C(=O)N(C)[C@@H](CCCCNC(=O)COCCOCCN(C)C(=O)COCCOCCN(C)C(=O)CC[C@H](C(=O)O)N(C)C(=O)CCCCCCCCCCCCCCCCC(=O)O)C(N)=O)CCOCC1)C(C)(C)S]

### Peptide Cyclization and Purification:

Crude peptide intermediate 1 (620 mg, 0.215 mmol) was dissolved in 20% MeCN/H2O (300 mL). To a stirred solution of the peptide was added iodine solution in MeOH (0.1M, 1.5 mL) drop-wise until the color of the solution remained yellow. After ~2 h LCMS showed the reaction was complete. Excess iodine was quenched by the addition of 1M Na₂S₂O₃ in water (15 uL) (turned colorless instantly). Then was added 10-20 mL of MeCN to decrease turbidity. The solution was purified by Prep-HPLC (A: 0.075% TFA in H₂O, B: ACN) (Note 1: Method A) to give the peptide of SEQ ID NO: 80 (169.4 mg, 97.85% purity, 23.9% yield for this step; over all yield: 17.1%) as a white solid. Analysis was performed using a C18 column with a flow rate of 1 mL/min (Note 2).

LCMS summary: Method: 10-80-3 min-1.5.amx, retention time: 2.033 min, calculated MW: 2883.42, observed MW: 1442.7[(M+2H)/2].

### Example 3: Synthesis of SEQ ID NO: 3

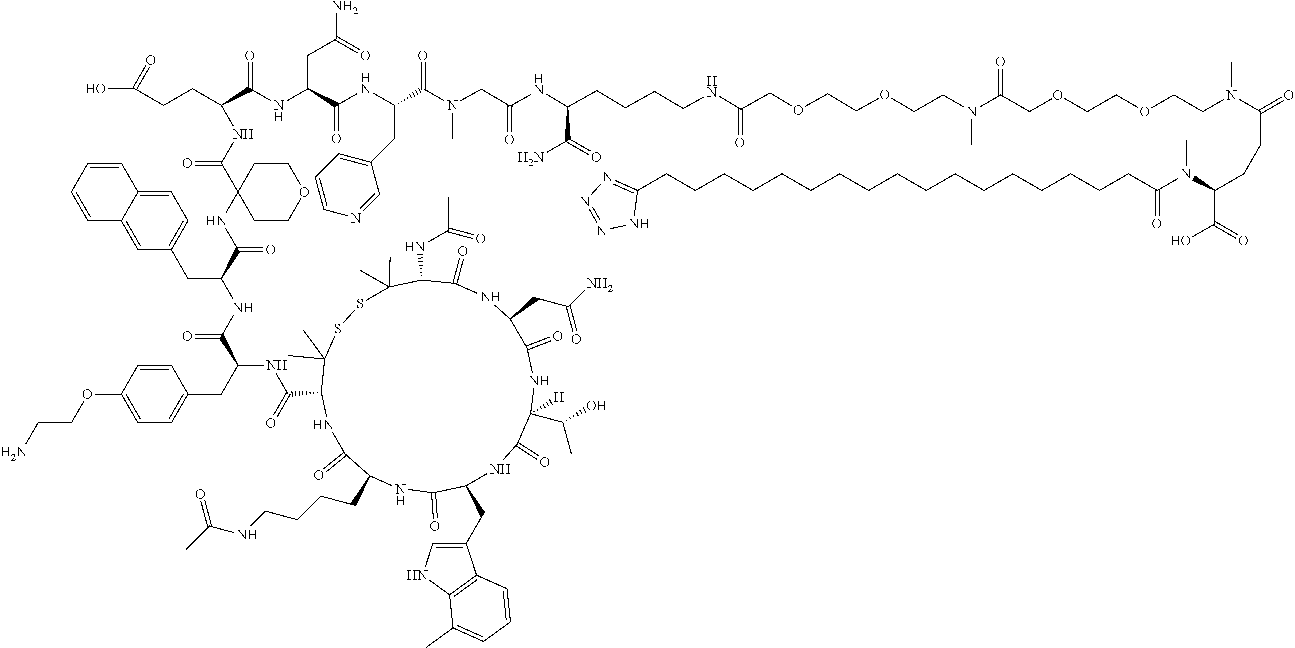 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N[C@@H](CCCCNC(=O)COCCOCCN(C)C(=O)COCCOCCN(C)C(=O)CC[C@@H](C(=O)O)N(C)C(=O)CCCCCCCCCCCCCCCCCc3nnn[nH]3)C(N)=O)CCOCC2)NC1=O]

The peptide was synthesized using standard Fmoc chemistry.
1) DMF and MBHA Resin (0.3 mmol, 0.90 g, sub: 0.33 mmol/g) were combined in a vessel, and the resin was allowed to swell for two hours.
2) A solution of 20% piperidine/DMF was added and the suspension was mixed for 30 min.
3) The resin was then drained and washed with DMF for 30 seconds*5 times.
4) Fmoc-amino acid solution was then added and mixed with the resin for 30 seconds before addition of a solution of HATU and DIEA in DMF. The reaction was allowed to proceed under nitrogen for 1-4 hours.
5) A solution of 20% piperidine/DMF was added and the suspension was mixed for 30 min.
6) Steps 2 to 5 were repeated for subsequent amino acid couplings. The coupling reactions were monitored by ninhydrin or tetrachlor color test, and upon completion, the resin was washed with DMF 5 times. Once peptide synthesis was complete, the resin was washed with MeOH 3 times and dried by vacuum.

### Monitoring Method:

1. Ninhydrin test: A: 5% ninhydrin/EtOH; B: 80% phenol/EtOH; C: pyridine
2. Tetrachlor color test: A: 2% tetrachlor/DMF; B: 2% aldehyde/DMF 110° C. for 3 min
   Synthetic Method for Removal of ivDde Protecting Group:

Deprotection of ivDde on NMeLys: The resin was washed with 50 mL of DMF (5×0.1 min) followed by addition of 3% hydrazine hydrate in DMF (50 mL) for 20 min*3 times (ninhydrin color reaction), and washed with DMF 5 times.

### Peptide Cleavage:

1) To the flask containing the side chain protected peptide at room temperature was added 30 mL cleavage buffer (5.0% DTT/2.5% H₂O/2.5% TIS/90% TFA), and mixture was stirred for 3 hrs.
2) The mixture was filtered and washed with 5 mL TFA. The combined filtrate was triturated with cold methyl tertbutyl ether (MTBE). The mixture was centrifuged (3000 rpm, 3 min) and decanted. The pellet was washed with MTBE and centrifuged.
3) The residue was lyophilized to give intermediate 2 (600 mg, 70.8% yield, crude)

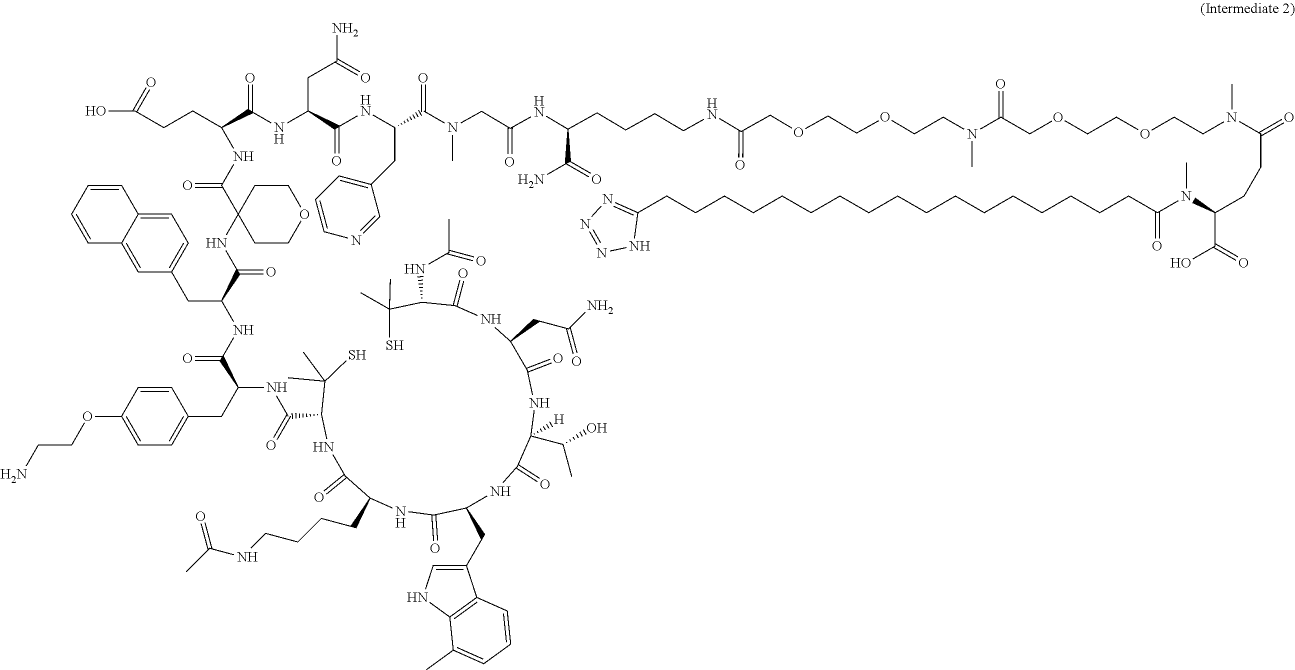 [SMILES: CC(=O)NCCCC[C@H](NC(=O)[C@H](Cc1c[nH]c2c(C)cccc12)NC(=O)[C@@H](NC(=O)[C@H](CC(N)=O)NC(=O)[C@@H](NC(C)=O)C(C)(C)S)[C@@H](C)O)C(=O)N[C@H](C(=O)N[C@@H](Cc1ccc(OCCN)cc1)C(=O)N[C@@H](Cc1ccc2ccccc2c1)C(=O)NC1(C(=O)N[C@@H](CCC(=O)O)C(=O)N[C@@H](CC(N)=O)C(=O)N[C@@H](Cc2cccnc2)C(=O)N(C)CC(=O)N[C@@H](CCCCNC(=O)COCCOCCN(C)C(=O)COCCOCCN(C)C(=O)CC[C@@H](C(=O)O)N(C)C(=O)CCCCCCCCCCCCCCCCCc2nnn[nH]2)C(N)=O)CCOCC1)C(C)(C)S]

### Peptide Cyclization and Purification:

Crude peptide intermediate 2 (600 mg, 0.212 mmol) was dissolved in 20% MeCN/H2O (300 mL). To a stirred solution of the peptide was added iodine solution in MeOH (0.1M, 1.5 mL) drop-wise until the color of the solution remains yellow. After ~2 h LCMS showed the reaction was complete. Excess iodine was quenched by the addition of 1M Na₂S₂O₃ in water (15 uL) (turned colorless instantly). Added 10-20 mL of MeCN to decrease turbidity. Purified the solution by Prep-HPLC (A: 0.075% TFA in H₂O, B: ACN) (Note 1: Method A) to give the peptide of SEQ ID NO: 3 (77.4 mg, 98.8% purity, 11.3% yield for this step; over all yield: 8.06%) obtained as white solid. Analysis was performed using a C18 column with a flow rate of 1 mL/min (Note 2).

LCMS summary: Method: 10-80-2 min-1.5_P2.amx, retention time: 1.607 min, calculated MW: 2822.34, observed MW: 941.7[(M+3H)/3].

### Example 4: IL23R Reporter Assay

Compounds were serially diluted in 100% (v/v) DMSO) and plated using an Echo acoustic dispenser (Labcyte) into 1536-well non-treated black assay plates (Corning #9146). 3 μL of HEK293 cells containing IL-23R, IL-12Rβ1 and a firefly luciferase reporter gene driven by a STAT-inducible promoter (Promega) were added to the plates (4000 cells/well), followed by 3 μL of 10 ng/mL IL-23 (equivalent to EC₉₀ concentration). After 5 h at 37° C., 5% CO₂, 95% relative humidity, cells were placed at 20° C. and treated with BioGlo reagent (Promega) according to the Manufacturer's instructions. Luminescence was measured on a Pherastar FSX (BMG LabTech). Data were normalized to IL-23 treatment (0% inhibition) and 30 μM of control inhibitor (100% inhibition), and IC₅₀ values were determined using a 4-parameter Hill equation. Data for exemplary compounds are shown below.
A: IC₅₀<0.05 μM;
B: 0.05 μM≤IC₅₀<0.5 μM;
C: 0.5 μM≤IC₅₀
ND: Not determined

TABLE 8
IL-23 Binding Data

| SEQ ID NO | IC₅₀ |
|---|---|
| 1 | A |
| 2 | B |
| 3 | A |
| 4 | A |
| 5 | A |
| 6 | A |
| 7 | A |
| 8 | A |
| 9 | A |
| 10 | A |
| 11 | B |
| 12 | A |
| 13 | B |
| 14 | ND |
| 15 | B |
| 16 | ND |
| 17 | ND |
| 18 | B |
| 19 | B |
| 20 | B |
| 21 | A |
| 22 | A |
| 23 | ND |
| 24 | B |
| 25 | B |
| 26 | A |
| 27 | A |
| 28 | B |
| 29 | B |
| 30 | A |
| 31 | B |
| 32 | B |
| 33 | A |
| 34 | A |
| 35 | A |
| 36 | B |
| 37 | A |
| 38 | A |
| 39 | A |
| 40 | ND |
| 41 | B |
| 42 | A |
| 43 | A |
| 44 | A |
| 45 | B |
| 46 | A |
| 47 | A |
| 48 | B |
| 49 | B |
| 50 | B |
| 51 | C |
| 52 | B |
| 53 | B |
| 54 | B |
| 55 | B |
| 56 | A |
| 57 | A |
| 58 | B |
| 59 | B |
| 60 | A |
| 61 | A |
| 62 | A |
| 63 | C |
| 64 | C |
| 65 | C |
| 66 | C |
| 67 | B |
| 68 | C |
| 69 | B |
| 70 | A |
| 71 | B |
| 72 | B |
| 73 | B |
| 74 | A |
| 75 | A |
| 76 | A |
| 77 | B |
| 78 | B |
| 79 | B |
| 80 | B |
| 81 | B |
| 82 | A |
| 83 | A |
| 84 | A |
| 85 | ND |
| 86 | ND |
| 87 | B |
| 88 | A |
| 89 | A |
| 90 | B |
| 91 | A |
| 92 | B |
| 93 | B |
| 94 | A |
| 95 | A |
| 96 | A |
| 97 | A |
| 98 | A |
| 99 | A |
| 100 | A |
| 101 | A |
| 102 | A |
| 103 | A |
| 104 | A |
| 105 | A |
| 106 | B |
| 107 | A |
| 108 | A |
| 109 | A |
| 110 | B |
| 111 | A |
| 112 | B |
| 113 | A |
| 114 | A |
| 115 | A |
| 116 | A |
| 117 | A |
| 118 | A |
| 119 | A |
| 120 | A |
| 121 | B |
| 122 | B |
| 123 | A |
| 124 | A |
| 125 | A |
| 126 | A |
| 127 | B |
| 128 | B |
| 129 | A |
| 130 | A |
| 131 | A |
| 132 | A |
| 133 | A |
| 134 | A |
| 135 | B |
| 136 | B |
| 137 | A |
| 138 | A |
| 139 | A |
| 140 | B |
| 141 | A |
| 142 | A |
| 143 | C |
| 144 | A |
| 145 | B |
| 146 | B |
| 147 | C |
| 148 | B |
| 149 | C |
| 150 | C |
| 151 | A |
| 152 | B |
| 153 | B |
| 154 | A |
| 155 | A |
| 156 | B |
| 157 | B |
| 158 | B |
| 159 | A |
| 160 | B |
| 161 | A |
| 162 | B |

### Example 5: PBMC pSTAT3 Assay

Cryopreserved peripheral blood mononuclear cells (PBMCs) from healthy donors were thawed and washed twice in ImmunoCult-XF T cell expansion medium (XF-TCEM) supplemented with CTL anti-aggregate wash. The cells were counted, resuspended at 2-6×10⁵ cells per mL XF-TCEM supplemented with penicillin/streptomycin and 100 ng/mL IL-1β (BioLegend, 579404), and cultured in tissue culture flasks coated with anti-CD3 (eBioscience, 16-0037-85 or BD Pharmingen, 555329) at 37° C. in 5% CO2. On day 4 of culture, PBMCs were collected, washed twice in RPMI-1640 supplemented with 0.1% BSA (RPMI-BSA), and incubated in RPMI-BSA in upright tissue culture flasks for ~4 hours at 37° C. in 5% CO2. Following this ‘starvation,’ a total of 6×104 cells in 30 μL RPMI-BSA was transferred into each well of a 384-well plate pre-spotted with peptide or DMSO. The cells were incubated for 30 minutes prior to the addition of TL-23 at a final concentration of 5 ng/mL. The cells were stimulated with cytokine for 30 minutes at 37° C. in 5% CO2, transferred onto ice for 10 minutes, and lysed. Cell lysates were stored at −80° C. until phosphorylated STAT3 was measured using the phospho-STAT panel kit (Meso Scale Discovery, K15202D). Results are provided below.
A: IC₅₀<0.5 nM;
B: 0.5 nM≤IC₅₀<5 nM;
C: 5 nM≤IC₅₀;
ND: Not determined

TABLE 9
PBMC pSTAT3 Assay Results

| SEQ ID NO | PBMC pSTAT3 IC50 |
|---|---|
| 1 | ND |
| 2 | A |
| 3 | A |
| 4 | A |
| 5 | B |
| 6 | ND |
| 7 | A |
| 8 | A |
| 9 | A |
| 10 | B |
| 11 | B |
| 12 | B |
| 13 | B |
| 14 | A |
| 15 | B |
| 16 | A |
| 17 | A |
| 18 | A |
| 19 | A |
| 20 | A |
| 21 | A |
| 22 | A |
| 23 | A |
| 24 | B |
| 25 | B |
| 26 | A |
| 27 | A |
| 28 | A |
| 29 | ND |
| 30 | A |
| 31 | A |
| 32 | A |
| 33 | A |
| 34 | A |
| 35 | A |
| 36 | A |
| 37 | A |
| 38 | A |
| 39 | A |
| 40 | B |
| 41 | A |
| 42 | A |
| 43 | A |
| 44 | A |
| 45 | A |
| 46 | A |
| 47 | A |
| 48 | A |
| 49 | A |
| 50 | A |
| 51 | A |
| 52 | A |
| 53 | A |
| 54 | A |
| 55 | A |
| 56 | A |
| 57 | A |
| 58 | A |
| 59 | A |
| 60 | A |
| 61 | A |
| 62 | A |
| 63 | C |
| 64 | B |
| 65 | ND |
| 66 | B |
| 67 | B |
| 68 | ND |
| 69 | ND |
| 70 | A |
| 71 | A |
| 72 | A |
| 73 | A |
| 74 | A |
| 75 | A |
| 76 | A |
| 77 | A |
| 78 | A |
| 79 | A |
| 80 | A |
| 81 | A |
| 82 | A |
| 83 | A |
| 84 | A |
| 85 | A |
| 86 | A |
| 87 | B |
| 88 | A |
| 89 | A |
| 90 | A |
| 91 | A |
| 92 | A |
| 93 | ND |
| 94 | A |
| 95 | A |
| 96 | A |
| 97 | A |
| 98 | A |
| 99 | A |
| 100 | A |
| 101 | A |
| 102 | A |
| 103 | A |
| 104 | A |
| 105 | A |
| 106 | B |
| 107 | A |
| 108 | A |
| 109 | A |
| 110 | A |
| 111 | A |
| 112 | B |
| 113 | A |
| 114 | A |
| 115 | B |
| 116 | A |
| 117 | A |
| 118 | A |
| 119 | A |
| 120 | A |
| 121 | A |
| 122 | A |
| 123 | A |
| 124 | A |
| 125 | B |
| 126 | A |
| 127 | B |
| 128 | B |
| 129 | A |
| 130 | A |
| 131 | A |
| 132 | A |
| 133 | A |
| 134 | A |
| 135 | ND |
| 136 | B |
| 137 | A |
| 138 | A |
| 139 | A |
| 140 | A |
| 141 | A |
| 142 | A |
| 143 | C |
| 144 | B |
| 145 | C |
| 146 | A |
| 147 | B |
| 148 | A |
| 149 | C |
| 150 | C |
| 151 | A |
| 152 | A |
| 153 | A |
| 154 | A |
| 155 | A |
| 156 | A |
| 157 | B |
| 158 | B |
| 159 | A |
| 160 | A |
| 161 | A |
| 162 | A |

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A polypeptide of Formula (I), comprising the amino acid sequence: 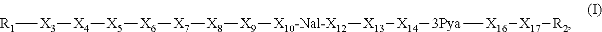
or a pharmaceutically acceptable salt thereof, wherein:
R₁ is MeCO, Z_{peg}, Z_{lipid}, succiniccarn, 5cpaCO, 8Aoc, 7Ahp, 6Ahx, 5Ava, or AEEP-Z_{lipid};
X₃ is R, K—Z_{lipid}, Dab-Z_{lipid}, NMeK—Z_{lipid}, a ring-forming amino acid, or absent;
X₄ is any amino acid;
X₅ is N, N(NMe₂), K(d), K—Z_{lipid}, Dab-Z_{lipid}, NMeK—Z_{lipid}, or a ring-forming amino acid;
X₆ is T, K—Z_{lipid}, NMeK—Z_{lipid}, Dab-Z_{lipid}, or a ring-forming amino acid;
X₇ is 7MeW, W, or absent;
X₈ is K(Ac), K(d), K(NMeAc), Q, K—Z_{lipid}, K—Z_{peg}, NMeK—Z_{lipid}, Dab-Z_{lipid}, Dab(NMecarn), Dab-Z_{peg}, or a ring-forming amino acid;
X₉ is any amino acid;
X₁₀ is AEF, TMAPF, AEF(d), TMAPF-Z_{lipid}, APEG3F, AEF-Z_{lipid}, or a ring-forming amino acid;
X₁₂ is THP, K—Z_{lipid}, Dab-Z_{lipid}, NMeK—Z_{lipid}, or a ring-forming amino acid;
X₁₃ is E, K(Ac), K(d), K—Z_{peg}, K(NMeAc), Dab(NMecarn), E(OAll), K—Z_{lipid}, Dab-Z_{lipid}, NMeK—Z_{lipid}, absent, or a ring-forming amino acid;
X₁₄ is N, K—Z_{lipid}, NMeK—Z_{lipid}, Dab-Z_{lipid}, or a ring-forming amino acid;
X₁₆ is Sar, NMeK(d), K—Z_{lipid}, Dab-Z_{lipid}, NMeK—Z_{lipid}, or a ring-forming amino acid;
X₁₇ is K—Z_{lipid}, Dab-Z_{lipid}, NMeK—Z_{lipid}, a ring-forming amino acid, or absent;
R₂ is CONH2, CONMe2, CONH—Z_{peg}, or CO—Z_{lipid};
Z_{peg}, independently for each occurrence, is a polyethylene glycol chain;
Z_{lipid}, independently for each occurrence, is a lipophilic substituent;
wherein the polypeptide comprises a first ring-forming amino acid that is linked to a second ring-forming amino acid to form a ring comprising 4-11 or 14 amino acids; or
wherein the polypeptide comprises a ring-forming amino acid that is linked to the C-terminus of the polypeptide to form a ring comprising 4-11 or 14 amino acids; and
wherein the polypeptide comprises at least one lipophilic substituent;
provided that the polypeptide is not selected from the group consisting of:
MeCO-r-Pen-N-T-7MeW-K (Ac)-Pen-AEF-2Nal-THP-E-N-3Pya-Sar-K (PEG2PEG2gEC18OH)-CONH2,
MeCO-r-Pen-N-T-7MeW-K (Ac)-Pen-AEF-2Nal-THP-E-N-3Pya-NMeK (PEG2PEG2gEC18OH)-CONH2,
MeCO-Pen-N-T-7MeW-K (Ac)-Pen-AEF-2Nal-THP-K (PEG2PEG2gEC18OH)-N-3Pya-Sar-CONH2,
MeCO-k (PEG2PEG2gEC18OH)-Pen-N-T-7MeW-K (Ac)-Pen-AEF-2Nal-THP-K (Ac)-N -3Pya-Sar-CONH2,
MeCO-r-Pen-N-T-7MeW-K (Ac)-Pen-AEF (PEG2PEG2gEC18OH)-2Nal-THP-K (Ac)-N -3Pya-Sar-CONH2,
MeCO-k (PEG2PEG2SP6gEC18OH)-Pen-N-T-7MeW-K (Ac)-Pen-AEF-2Nal-THP-K (Ac)-N-3Pya-Sar-CONH2,
MeCO-k (PEG2PEG2gEmXOH)-Pen-N-T-7MeW-K (Ac)-Pen-AEF-2Nal-THP-K (PEG2PEG2gEmXOH)-N-3Pya-Sar-CONH2,
MeCO-k (PEG2PEG2gEC16OH)-Pen-N-T-7MeW-K (Ac)-Pen-AEF-2Nal-THP-K (gEC16)-N-3Pya-Sar-CONH2,
HOC18gEPEG2PEG2-r-Pen-N-T-7MeW-K (Ac)-Pen-AEF-2Nal-THP-K (Ac)-N-3Pya-Sar -CONH2,
HOC18gEPEG2PEG2-r-Pen-N-T-7MeW-K (Ac)-Pen-AEF-2Nal-THP-K (PEG2PEG2gEC18OH)-N-3Pya-Sar-CONH2,
MeCO-k (PEG2PEG2gEDab (mXOH) 2)-Pen-N-T-7MeW-K (Ac)-Pen-AEF-2Nal-THP-K (Ac)-N-3Pya-Sar-CONH2,
MeCO-k (PEG2PEG2gE (c) C18OH)-Pen-N-T-7MeW-K (Ac)-Pen-AEF-2Nal-THP-K (Ac)-N-3Pya-Sar-CONH2,
MeCO-r-Pen-N-T-7MeW-K (Ac)-Pen-AEF-2Nal-THP-E-N-3Pya-Sar-K (PEG2PEG2gEC16OH)-CONH2,
MeCO-r-Pen-N-T-7MeW-K (Ac)-Pen-AEF-2Nal-THP-E-N-3Pya-K (PEG2PEG2gEC18OH)-CONH2,
MeCO-Pen-N-T-7MeW-K (Ac)-Pen-AEF-2Nal-THP-K (PEG2PEG2gEC16OH)-N-3Pya-Sar -CONH2,
MeCO-Pen-K (PEG2PEG2gEC18OH)-T-7MeW-K (Ac)-Pen-AEF-2Nal-THP-E-N-3Pya-Sar -CONH2,
MeCO-r-Pen-K (PEG2PEG2gEC16OH)-T-7MeW-K (Ac)-Pen-AEF-2Nal-THP-E-N-3Pya-Sar-CONH2,
MeCO-r-Pen-N-T-7MeW-K (Ac)-Pen-AEF-2Nal-THP-E-N-3Pya-NMeK (PEG2PEG2gEC16OH)-CONH2,
MeCO-r-Pen-N-T-7MeW-K (Ac)-Pen-AEF-2Nal-THP-E-N-3Pya-K (PEG2PEG2gEC16OH)-CONH2,
MeCO-k (PEG2PEG2gEC16OH)-Pen-N-T-7MeW-K (Ac)-Pen-AEF-2Nal-THP-K (Ac)-N-3Pya-Sar-CONH2,
MeCO-r-Pen-N-T-7MeW-K (Ac)-Pen-AEF (PEG2PEG2gEC16OH)-2Nal-THP-K (Ac)-N-3Pya-Sar-CONH2,
MeCO-r-Pen-N-T-7MeW-K (Ac)-Pen-AEF-2Nal-THP-K (PEG2PEG2gEC18OH)-N-3Pya-Sar-CONH2,
MeCO-r-Pen-N-T-7MeW-K (Ac)-Pen-AEF-2Nal-THP-K (PEG2PEG2gEC16OH)-N-3Pya-Sar-CONH2,
MeCO-Pen-N-T-7MeW-K (Ac)-Pen-AEF-2Nal-THP-K (gEC16OH)-N-3Pya-Sar-CONH2,
MeCO-Pen-N-T-7MeW-K (Ac)-Pen-AEF-2Nal-THP-K (gEC18OH)-N-3Pya-Sar-CONH2,
MeCO-Pen-N-T-7MeW-K (Ac)-Pen-AEF-2Nal-THP-K (gEC18)-N-3Pya-Sar-CONH2,
MeCO-Pen-N-T-7MeW-K (Ac)-Pen-AEF-2Nal-THP-K (PEG6gEC16OH)-N-3Pya-Sar-CONH2,
MeCO-Pen-N-T-7MeW-K (Ac)-Pen-AEF-2Nal-THP-K (PEG6gEC18OH)-N-3Pya-Sar-CONH2,
MeCO-Pen-N-T-7MeW-K (Ac)-Pen-AEF-2Nal-THP-K (PEG2gEC16OH)-N-3Pya-Sar-CONH2,
MeCO-Pen-N-T-7MeW-K (Ac)-Pen-AEF-2Nal-THP-K (PEG2gEC18OH)-N-3Pya-Sar-CONH2,
MeCO-Pen-N-T-7MeW-K (Ac)-Pen-AEF-2Nal-THP-K (PEG2PEG2gEC20OH)-N-3Pya-Sar-CONH2,
HOC16gEPEG2PEG2-r-Pen-N-T-7MeW-K (Ac)-Pen-AEF-2Nal-THP-K (PEG2PEG2gEC16OH)-N-3Pya-Sar-CONH2,
MeCO-Pen-N-T-7MeW-K (Ac)-Pen-AEF-2Nal-THP-K (PEG2PEG6gEC16OH)-N-3Pya-Sar-CONH2,
MeCO-Pen-N-T-7MeW-K (Ac)-Pen-AEF-2Nal-THP-K (PEG2PEG6gEC18OH)-N-3Pya-Sar-CONH2,
MeCO-Pen-N-T-7MeW-K (Ac)-Pen-AEF-2Nal-THP-K (PEG2PEG2C16OH)-N-3Pya-Sar-CONH2,
MeCO-Pen-N-T-7MeW-K (Ac)-Pen-AEF-2Nal-THP-K (PEG2PEG2C18OH)-N-3Pya-Sar-CONH2,
MeCO-Pen-N-T-7MeW-K (Ac)-Pen-AEF-2Nal-THP-K (PEG6PEG6gEC16OH)-N-3Pya-Sar-CONH2,
MeCO-Pen-N-T-7MeW-K (Ac)-Pen-AEF-2Nal-THP-K (PEG6PEG6gEC18OH)-N-3Pya-Sar-CONH2,
MeCO-Pen-N-T-7MeW-K (Ac)-Pen-AEF-2Nal-THP-K (PEG24-gEC16OH)-N-3Pya-Sar-CONH2,
MeCO-Pen-N-T-7MeW-K (Ac)-Pen-AEF-2Nal-THP-K (PEG24-gEC18OH)-N-3Pya-Sar-CONH2,
MeCO-Pen-N-T-7MeW-K (Ac)-Pen-AEF-2Nal-THP-K (PEG24-C18OH)-N-3Pya-Sar-CONH2,
MeCO-k (gEC18OH)-Pen-N-T-7MeW-K (Ac)-Pen-AEF-2Nal-THP-K (Ac)-N-3Pya-Sar-CONH2,
MeCO-k (PEG2gEC18OH)-Pen-N-T-7MeW-K (Ac)-Pen-AEF-2Nal-THP-K (Ac)-N-3Pya-Sar-CONH2,
MeCO-r-Pen-K (PEG2PEG2gEC18OH)-T-7MeW-K (Ac)-Pen-AEF-2Nal-THP-K (Ac)-N -3Pya-Sar-CONH2,
MeCO-r-Pen-K (gEC18OH)-T-7MeW-K (Ac)-Pen-AEF-2Nal-THP-K (Ac)-N-3Pya-Sar-CONH2,
MeCO-r-Pen-K (PEG2gEC18OH)-T-7MeW-K (Ac)-Pen-AEF-2Nal-THP-K (Ac)-N-3Pya-Sar-CONH2,
MeCO-r-Pen-K (PEG2PEG2C18OH)-T-7MeW-K (Ac)-Pen-AEF-2Nal-THP-K (Ac)-N-3Pya-Sar-CONH2,
MeCO-r-Pen-N-T-7MeW-K (Ac)-Pen-AEF-2Nal-THP-K (Ac)-N-3Pya-NMeK (PEG2PEG2gEC18OH)-CONH2,
MeCO-r-Pen-N-T-7MeW-K (Ac)-Pen-AEF-2Nal-THP-K (PEG2PEG2-DgEC18OH)-N-3Pya-Sar-CONH2,
MeCO-r-Pen-N-T-7MeW-K (Ac)-Pen-AEF-2Nal-THP-K (PEG2PEG2-pC18OH)-N-3Pya-Sar-CONH2,
MeCO-r-Pen-N-T-7MeW-K (Ac)-Pen-AEF-2Nal-THP-K (PEG2PEG2gETrxC18OH)-N-3Pya-Sar-CONH2,
MeCO-r-Pen-N-T-7MeW-K (Ac)-Pen-AEF-2Nal-THP-K (PEG2PEG2gETrxC20OH)-N-3Pya-Sar-CONH2,
MeCO-k (PEG6gEC18OH)-Pen-N-T-7MeW-K (Ac)-Pen-AEF-2Nal-THP-K (Ac)-N-3Pya-Sar-CONH2,
MeCO-k (PEG2PEG6gEC18OH)-Pen-N-T-7MeW-K (Ac)-Pen-AEF-2Nal-THP-K (Ac)-N-3Pya-Sar-CONH2,
MeCO-k (PEG2PEG2C18OH)-Pen-N-T-7MeW-K (Ac)-Pen-AEF-2Nal-THP-K (Ac)-N-3Pya-Sar-CONH2,
MeCO-r-Pen-K (PEG6gEC18OH)-T-7MeW-K (Ac)-Pen-AEF-2Nal-THP-K (Ac)-N-3Pya-Sar-CONH2,
MeCO-r-Pen-K (PEG2PEG6gEC18OH)-T-7MeW-K (Ac)-Pen-AEF-2Nal-THP-K (Ac)-N -3Pya-Sar-CONH2,
MeCO-r-Pen-N-T-7MeW-K (Ac)-Pen-AEF-2Nal-THP-K (PEG2PEG2-pppC18OH)-N-3Pya-Sar-CONH2,
MeCO-k (gEC16)-Pen-N-T-7MeW-K (Ac)-Pen-AEF-2Nal-THP-K (Ac)-N-3Pya-Sar-CONH2,
MeCO-k (gEC18)-Pen-N-T-7MeW-K (Ac)-Pen-AEF-2Nal-THP-K (Ac)-N-3Pya-Sar-CONH2,
MeCO-r-Pen-K (gEC16)-T-7MeW-K (Ac)-Pen-AEF-2Nal-THP-K (Ac)-N-3Pya-Sar-CONH2,
MeCO-r-Pen-K (gEC18)-T-7MeW-K (Ac)-Pen-AEF-2Nal-THP-K (Ac)-N-3Pya-Sar-CONH2,
MeCO-k (PEG2PEG2gETrxC18OH)-Pen-N-T-7MeW-K (Ac)-Pen-AEF-2Nal-THP-K (Ac)-N-3Pya-Sar-CONH2,
MeCO-k (PEG2PEG2gETrxC20OH)-Pen-N-T-7MeW-K (Ac)-Pen-AEF-2Nal-THP-K (Ac)-N-3Pya-Sar-CONH2,
MeCO-Pen-K (PEG2PEG2gETrxC18OH)-T-7MeW-K (Ac)-Pen-AEF-2Nal-THP-K (Ac)-N -3Pya-Sar-CONH2,
MeCO-Pen-K (PEG2PEG2gETrxC20OH)-T-7MeW-K (Ac)-Pen-AEF-2Nal-THP-K (Ac)-N -3Pya-Sar-CONH2,
MeCO-Pen-N-T-7MeW-K (Ac)-Pen-AEF-2Nal-THP-K (gEC16)-N-3Pya-Sar-CONH2,
MeCO-k (PEG2PEG2gEC16OH)-Pen-K (PEG2PEG2gEC16OH)-T-7MeW-K (Ac)-Pen-AEF -2Nal-THP-K (Ac)-N-3Pya-Sar-CONH2,
MeCO-k (PEG2PEG2gEC18OH)-Pen-K (PEG2PEG2gEC18OH)-T-7MeW-K (Ac)-Pen-AEF -2Nal-THP-K (Ac)-N-3Pya-Sar-CONH2,
MeCO-k (PEG2PEG2gEC20OH)-Pen-K (PEG2PEG2gEC20OH)-T-7MeW-K (Ac)-Pen-AEF -2Nal-THP-K (Ac)-N-3Pya-Sar-CONH2,
MeCO-k (gEC16)-Pen-K (gEC16)-T-7MeW-K (Ac)-Pen-AEF-2Nal-THP-K (Ac)-N-3Pya-Sar-CONH2,
MeCO-k (gEC18)-Pen-K (gEC18)-T-7MeW-K (Ac)-Pen-AEF-2Nal-THP-K (Ac)-N-3Pya-Sar-CONH2,
MeCO-k (PEG2PEG2gEC16)-Pen-N-T-7MeW-K (Ac)-Pen-AEF-2Nal-THP-K (Ac)-N-3Pya-Sar-CONH2,
MeCO-k (PEG2PEG2gEC18)-Pen-N-T-7MeW-K (Ac)-Pen-AEF-2Nal-THP-K (Ac)-N-3Pya-Sar-CONH2,
MeCO-Pen-K (PEG2PEG2gEC16)-T-7MeW-K (Ac)-Pen-AEF-2Nal-THP-K (Ac)-N-3Pya-Sar-CONH2,
MeCO-Pen-K (PEG2PEG2gEC18)-T-7MeW-K (Ac)-Pen-AEF-2Nal-THP-K (Ac)-N-3Pya-Sar-CONH2,
MeCO-Pen-K (1PEG21PEG2IsoGluC16Diacid)-T-7MeW-K (Ac)-Pen-AEF-2Nal-THP-K (Ac)-N-3Pya-Sar-CONH2,
MeCO-r-Pen-N-T-7MeW-K (PEG2PEG2gEC18OH)-Pen-AEF-2Nal-THP-K (Ac)-N-3Pya-Sar-CONH2,
MeCO-Pen-N-T-7MeW-K (Ac)-Pen-AEF-2Nal-THP-K (gEC20OH)-N-3Pya-Sar-CONH2,
MeCO-Pen-N-T-7MeW-K (Ac)-Pen-AEF-2Nal-THP-K (PEG2PEG2-TrxgEC18OH)-N-3Pya-Sar-CONH2,
MeCO-Pen-N-T-7MeW-K (Ac)-Pen-AEF-2Nal-THP-K (PEG2PEG2-TrxgETrxC20OH)-N-3Pya-Sar-CONH2,
MeCO-Pen-N-T-7MeW-K (Ac)-Pen-AEF-2Nal-THP-K (PEG2PEG2-TrxgEC20OH)-N-3Pya-Sar-CONH2,
MeCO-k (PEG2PEG2gEC10OH)-Pen-N-T-7MeW-K (Ac)-Pen-AEF-2Nal-THP-K (gEC16)-N-3Pya-Sar-CONH2,
MeCO-k (PEG2PEG2gEC18OH)-Pen-K (gEC16)-T-7MeW-K (Ac)-Pen-AEF-2Nal-THP-K (Ac)-N-3Pya-Sar-CONH2,
MeCO-r-Pen-K (PEG2PEG2pgEC18OH)-T-7MeW-K (Ac)-Pen-AEF-2Nal-THP-K (Ac)-N -3Pya-Sar-CONH2,
MeCO-Pen-N-T-7MeW-K (Ac)-Pen-AEF-2Nal-THP-K (PEG2PEG2gEmXOH)-N-3Pya-Sar-CONH2,
MeCO-k (PEG2PEG2gEC18OH)-Pen-N-T-7MeW-K (Ac)-Pen-AEF-2Nal-THP-K (gEC16)-N-3Pya-Sar-CONH2,
MeCO-r-Pen-K (PEG2PEG2pppgEC18OH)-T-7MeW-K (Ac)-Pen-AEF-2Nal-THP-K (Ac)-N-3Pya-Sar-CONH2,
MeCO-k (PEG2PEG2PgEC18OH)-Pen-N-T-7MeW-K (Ac)-Pen-AEF-2Nal-THP-K (Ac)-N -3Pya-Sar-CONH2,
MeCO-k (PEG2PEG2PPPgEC18OH)-Pen-N-T-7MeW-K (Ac)-Pen-AEF-2Nal-THP-K (Ac)-N-3Pya-Sar-CONH2,
MeCO-Pen-N-T-7MeW-K (Ac)-Pen-AEF-2Nal-THP-K (PEG2PEG2PgEC18OH)-N -3Pya-Sar-CONH2,
MeCO-Pen-N-T-7MeW-K (Ac)-Pen-AEF-2Nal-THP-K (PEG2PEG2PPPgEC18OH)-N -3Pya-Sar-CONH2,
MeCO-k (PEG2PEG2-TrxgEC18OH)-Pen-N-T-7MeW-K (Ac)-Pen-AEF-2Nal-THP-K (Ac)-N-3Pya-Sar-CONH2,
MeCO-k (PEG2PEG2gEmXOH)-Pen-N-T-7MeW-K (Ac)-Pen-AEF-2Nal-THP-K (Ac)-N -3Pya-Sar-CONH2,
MeCO-Pen-N-T-7MeW-K (Ac)-Pen-AEF-2Nal-THP-K (PEG2PEG2gEpXOH)-N-3Pya-Sar-CONH2,
MeCO-k (PEG2PEG2gEpXOH)-Pen-N-T-7MeW-K (Ac)-Pen-AEF-2Nal-THP-K (PEG2PEG2gEpXOH)-N-3Pya-Sar-CONH2,
MeCO-k (PEG2PEG2gEmXOH)-Pen-N-T-7MeW-K (Ac)-Pen-AEF-2Nal-THP-K (gEC16)-N-3Pya-Sar-CONH2,
MeCO-Pen-N-T-7MeW-K (Ac)-Pen-AEF-2Nal-THP-K (DAP (C16OH) 2)-N-3Pya-Sar-CONH2,
MeCO-k (DAP (C16OH) 2)-Pen-N-T-7MeW-K (Ac)-Pen-AEF-2Nal-THP-K (Ac)-N-3Pya-Sar-CONH2,
MeCO-Pen-N-T-7MeW-K (Ac)-Pen-AEF-2Nal-THP-K (PEG2PEG2gEDAP (C16OH) 2)-N -3Pya-Sar-CONH2,
MeCO-k (PEG2PEG2gEDAP (C16OH) 2)-Pen-N-T-7MeW-K (Ac)-Pen-AEF-2Nal-THP-K (Ac)-N-3Pya-Sar-CONH2,
MeCO-Pen-N-T-7MeW-K (Ac)-Pen-AEF-2Nal-THP-K (PEG2PEG2Sp6gEC18OH)-N -3Pya-Sar-CONH2,
MeCO-Pen-N-T-7MeW-K (Ac)-Pen-AEF-2Nal-THP-K (PEG2Sp6PEG2gEC18OH)-N -3Pya-Sar-CONH2,
MeCO-k (PEG2PEG2gEC16OH)-Pen-N-T-7MeW-K (Ac)-Pen-AEF-2Nal-THP-K (gEC18)-N-3Pya-Sar-CONH2,
MeCO-k (PEG2PEG2gEC18OH)-Pen-N-T-7MeW-K (Ac)-Pen-AEF-2Nal-THP-K (gEC18)-N-3Pya-Sar-CONH2,
MeCO-Pen-K (1PEG21PEG2IsoGluC18Diacid)-T-7MeW-K (Ac)-Pen-AEF-2Nal-THP-K (Ac)-N-3Pya-Sar-CONH2,
MeCO-Pen-K (PEG2PEG2gEC18OH)-T-7MeW-K (Ac)-Pen-AEF-2Nal-THP-K (PEG2PEG2gESp6C18OH)-N-3Pya-Sar-CONH2,
MeCO-Pen-K (PEG2PEG2gEC18OH)-T-7MeW-K (Ac)-Pen-AEF-2Nal-THP-K (Sp6PEG2PEG2gEC18OH)-N-3Pya-Sar-CONH2,
MeCO-k (gEC16)-Pen-N-T-7MeW-K (Ac)-Pen-AEF-2Nal-THP-K (gEC18)-N-3Pya-Sar-CONH2,
MeCO-k (gEC18)-Pen-N-T-7MeW-K (Ac)-Pen-AEF-2Nal-THP-K (gEC18)-N-3Pya-Sar-CONH2,
MeCO-k (PEG2PEG2gEC10OH)-Pen-N-T-7MeW-K (Ac)-Pen-AEF-2Nal-THP-K (gEC18)-N-3Pya-Sar-CONH2,
MeCO-Pen-K (PEG2PEG2gEC18OH)-T-7MeW-K (Ac)-Pen-AEF-2Nal-THP-K (PEG2PEG2gETrxC20OH)-N-3Pya-Sar-CONH2,
MeCO-k (PEG2PEG2gESp6C18OH)-Pen-N-T-7MeW-K (Ac)-Pen-AEF-2Nal-THP-K (Ac)-N-3Pya-Sar-CONH2,
MeCO-k (PEG2Sp6PEG2gEC18OH)-Pen-N-T-7MeW-K (Ac)-Pen-AEF-2Nal-THP-K (Ac)-N-3Pya-Sar-CONH2,
MeCO-k (Sp6PEG2PEG2gEC18OH)-Pen-N-T-7MeW-K (Ac)-Pen-AEF-2Nal-THP-K (Ac)-N-3Pya-Sar-CONH2,
MeCO-K [PEG2PEG2-gEDAP (pXOH) 2] -Pen-N-T-7MeW-K (Ac)-Pen-AEF-2Nal-THP-K (Ac)-N-3Pya-Sar-CONH2,
MeCO-Pen-N-T-7MeW-K (Ac)-Pen-AEF-2Nal-THP-K [PEG2PEG2-gEDAP-(mXOH) 2] -N-3Pya-Sar-CONH2,
MeCO-k (GolAC16)-Pen-N-T-7MeW-K (Ac)-Pen-AEF-2Nal-THP-K (Ac)-N-3Pya-Sar-CONH2,
MeCO-k (GolAC16OH)-Pen-N-T-7MeW-K (Ac)-Pen-AEF-2Nal-THP-K (Ac)-N-3Pya-Sar-CONH2,
MeCO-k (GolAC18OH)-Pen-N-T-7MeW-K (Ac)-Pen-AEF-2Nal-THP-K (Ac)-N-3Pya-Sar-CONH2,
MeCO-k (PEG2PEG2 GolAC18OH)-Pen-N-T-7MeW-K (Ac)-Pen-AEF-2Nal-THP-K (Ac)-N-3Pya-Sar-CONH2,
MeCO-k (PEG2PEG2-C18GolB)-Pen-N-T-7MeW-K (Ac)-Pen-AEF-2Nal-THP-K (Ac)-N -3Pya-Sar-CONH2,
MeCO-k (PEG2PEG2gE (C) C18OH)-Pen-N-T-7MeW-K (Ac)-Pen-AEF-2Nal-THP-K (Ac)-N-3Pya-Sar-CONH2,
MeCO-k (PEG2PEG2gEC18OH (C))-Pen-N-T-7MeW-K (Ac)-Pen-AEF-2Nal-THP-K (Ac)-N-3Pya-Sar-CONH2,
MeCO-Pen-N-T-7MeW-K (Ac)-Pen-AEF-2Nal-THP-E-N-3Pya-Sar-k (PEG12_IsoGlu_C18_Diacid)-CONH2,
MeCO-Pen-N-T-7MeW-K (Ac)-Pen-AEF-2Nal-THP-E-N-3Pya-Sar-k (Peg4_IsoGlu_Palm)-CONH2,
MeCO-Pen-N-T-7MeW-K (Ac)-Pen-AEF-2Nal-THP-E-N-3Pya-Sar-k (IsoGlu_Palm)-CONH2,
MeCO-Pen-N-T-7MeW-K (Ac)-Pen-AEF-2Nal-THP-E-N-3Pya-Sar-k (PEG12_C18_Diacid)-CONH2,
MeCO-Pen-N-T-7MeW-K (Ac)-Pen-AEF-2Nal-THP-E-N-3Pya-Sar-k (Peg4_IsoGlu_C18_Diacid)-CONH2,
MeCO-Pen-N-T-7MeW-K (Ac)-Pen-AEF-2Nal-THP-E-N-3Pya-Sar-k (PEG12_IsoGlu_Palm)-CONH2,
MeCO-Pen-N-T-7MeW-K (Ac)-Pen-AEF-2Nal-THP-E-N-3Pya-Sar-k (Peg4_C18_Diacid)-CONH2,
MeCO-Pen-N-T-7MeW-K (Ac)-Pen-AEF-2Nal-THP-E-N-3Pya-Sar-k (IsoGlu_C18_Diacid)-CONH2,
MeCO-r-Pen-K (1PEG21PEG2IsoGluC16Diacid)-T-7MeW-K (Ac)-Pen-AEF-2Nal-THP-E -N-3Pya-Sar-CONH2,
MeCO-r-Pen-K (1PEG21PEG2IsoGluC18Diacid)-T-7MeW-K (Ac)-Pen-AEF-2Nal-THP-E -N-3Pya-Sar-CONH2,
MeCO-Pen-N-T-7MeW-K (Ac)-Pen-AEF-2Nal-THP-K (1PEG21PEG2IsoGluC16Diacid)-N-3Pya-Sar-CONH2,
MeCO-Pen-N-T-7MeW-K (Ac)-Pen-AEF-2Nal-THP-K (1PEG21PEG2IsoGluC18Diacid)-N-3Pya-Sar-CONH2,
MeCO-r-Pen-N-T-7MeW-K (Ac)-Pen-AEF-2Nal-THP-E-N-3Pya-K (1PEG21PEG2IsoGluC18Diacid)-CONH2,
MeCO-r-Pen-N-T-7MeW-K (Ac)-Pen-AEF-2Nal-THP-E-N-3Pya-Sar-K (1PEG21PEG2IsoGluC16Diacid)-CONH2,
MeCO-r-Pen-N-T-7MeW-K (Ac)-Pen-AEF-2Nal-THP-E-N-3Pya-Sar-K (1PEG21PEG2IsoGluC18Diacid)-CONH2,
HOC10gEPEG2COPEG2CO-r-Pen-N-T-7MeW-K (Ac)-Pen-AEF-2Nal-THP-K (PEG2PEG2gEC10OH)-N-3Pya-Sar-CONH2,
MeCO-Pen-N-T-7MeW-K (Ac)-Pen-AEF-2Nal-THP-K (PEG2PEG2gEC10OH)-N-3Pya-Sar-CONH2,
MeCO-k (PEG2PEG2gEC10OH)-Pen-N-T-7MeW-K (Ac)-Pen-AEF-2Nal-THP-K (Ac)-N -3Pya-Sar-CONH2,
MeCO-Pen-K (PEG2PEG2gEC10OH)-T-7MeW-K (Ac)-Pen-AEF-2Nal-THP-K (Ac)-N -3Pya-Sar-CONH2,
MeCO-Pen-N-T-7MeW-K (Ac)-Pen-AEF-2Nal-THP-K (gEC10OH)-N-3Pya-Sar-CONH2,
MeCO-k (PEG2PEG2gEC10OH)-Pen-K (PEG2PEG2gEC10OH)-T-7MeW-K (Ac)-Pen-AEF -2Nal-THP-K (Ac)-N-3Pya-Sar-CONH2,
HOC16gEPEG2PEG2orn (2)-r-Pen-N-T-7MeW-K (Ac)-Pen-AEF-2Nal-THP-E (2)-N -3Pya-Sar-CONH2,
MeCO-k (PEG2PEG2gEC12OH (c))-Pen-N-T-7MeW-K (Ac)-Pen-AEF-2Nal-THP-K (Ac)-N-3Pya-Sar-CONH2,
MeCO-k (PEG2PEG2gE (C) C12)-Pen-N-T-7MeW-K (Ac)-Pen-AEF-2Nal-THP-K (Ac)-N -3Pya-Sar-CONH2,
MeCO-r-Pen-N-T-7MeW-K (Ac)-Pen-AEF-2Nal-THP-K (FITCPEG4)-N-3Pya-Sar-K (PEG2PEG2gEC18OH)-CONH2,
succiniccarn-k (PEG2PEG2gEC18OH)-Pen-N-T-7MeW-K (Ac)-Pen-AEF-2Nal-THP-E-N -3Pya-Sar-CONH2,
MeCO-r-Pen-K (PEG2PEG2gEC18OH)-T-7MeW-K (Ac)-Pen-dAEF-2Nal-THP-E-N-3Pya-Sar-CONH2,
MeCO-Pen-N-T-W-K (PEG2PEG2gEC20OH)-Pen-AEF-2Nal-THP-K (Ac)-N-3Pya-Sar-CONH2,
MeCO-Pen-K (PEG2PEG2gEC20OH)-T-W-K (Ac)-Pen-AEF-2Nal-THP-K (Ac)-N-3Pya-Sar-CONH2,
MeCO-K (PEG2PEG2gEC20OH)-Pen-N-T-W-K (Ac)-Pen-AEF-2Nal-THP-K (Ac)-N-3Pya-Sar-CONH2,
MeCO-Pen-N-T-W-K (Ac)-Pen-AEF-2Nal-THP-K (Ac)-N-3Pya-Sar-K (PEG2PEG2gEC20OH)-CONH2,
MeCO-r-Pen-N-T-7MeW-K (PEG2PEG2gEC20OH)-Pen-AEF-2Nal-THP-E-N-3Pya-Sar-CONH2,
cPEG3aCO-r-Pen-N-T-7MeW-K (PEG2PEG2gEC20OH)-Pen-AEF-2Nal-THP-E-N-3Pya-Sar-CONH2,
cPEG3aCO-r-Pen-K (PEG2PEG2gEC20OH)-T-7MeW-K (Ac)-Pen-AEF-2Nal-THP-E-N-3Pya-Sar-CONH2,
MeCO-r-Pen-N-T-7MeW-K (Ac)-Pen-AEF-2Nal-THP-E-N-3Pya-Sar-K (PEG2PEG2gEC20OH)-CONH2,
MeCO-r-Abu-N-T-7MeW-K (Ac)-aMeC-AEF-2Nal-THP-E-N-3Pya-Sar-K (PEG2PEG2gEC18OH)-CONH2,
MeCO-r-Abu-N-T-7MeW-K (Ac)-Pen-AEF-2Nal-THP-E-N-3Pya-Sar-K (PEG2PEG2gEC18OH)-CONH2,
MeCO-Pen-N-T-7MeW-K (PEG2PEG2gEC12)-Pen-AEF-2Nal-THP-K (PEG2PEG2gEC12)-N-3Pya-Sar-CONH2,
MeCO-Pen-N-T-7MeW-K (PEG2PEG2gEC14)-Pen-AEF-2Nal-THP-K (PEG2PEG2gEC14)-N-3Pya-Sar-CONH2,
MeCO-k (PEG2PEG2gEC12)-Pen-N-T-7MeW-K (Ac)-Pen-AEF-2Nal-THP-E-N-3Pya-Sar-CONH2,
MeCO-k (PEG2PEG2gEC14)-Pen-N-T-7MeW-K (Ac)-Pen-AEF-2Nal-THP-E-N-3Pya-Sar-CONH2,
MeCO-r-Pen-N-T-7MeW-K (PEG2PEG2gEC12)-Pen-AEF-2Nal-THP-E-N-3Pya-Sar-K (PEG2PEG2gEC12)-CONH2,
MeCO-r-Pen-N-T-7MeW-K (PEG2PEG2gEC12)-Pen-AEF-2Nal-THP-K (PEG2PEG2gEC12)-N-3Pya-Sar-CONH2,
MeCO-r-Pen-N-T-7MeW-K (Ac)-Pen-AEF-2Nal-THP-K (PEG2PEG2gEC12)-N-3Pya-Sar-CONH2,
MeCO-r-Pen-N-T-7MeW-K (PEG2PEG2gEC12)-Pen-AEF-2Nal-THP-E-N-3Pya-Sar-CONH2,
MeCO-r-Pen-N-T-7MeW-K (Ac)-Pen-AEF-2Nal-THP-E-N-3Pya-Sar-K (PEG2PEG2gEC12)-CONH2,
cPEG3aCO-k (PEG2PEG2PEG2PEG2gEC12)-Pen-N (N (Me) 2)-T-7MeW-K (Ac)-Pen-TMAPF -2Nal-THP-K (NMeAc)-N-3Pya-NMeK (PEG2PEG2PEG2PEG2gEC12)-CONH2,
MeCO-Pen-N-T-7MeW-K (PEG2PEG2gEC14)-Pen-AEF-2Nal-THP-E-N-3Pya-Sar-CONH2,
MeCO-Pen-K (PEG2PEG2gEC14)-T-7MeW-K (Ac)-Pen-AEF-2Nal-THP-K (Ac)-N-3Pya-Sar-CONH2,
MeCO-Pen-N-T-7MeW-K (PEG2PEG2gEC12)-Pen-AEF-2Nal-THP-K (Ac)-N-3Pya-Sar-CONH2,
MeCO-Pen-N-T-7MeW-K (Ac)-Pen-AEF-2Nal-THP-K (PEG2PEG2gEC14)-N-3Pya-Sar-CONH2,
MeCO-Pen-K (PEG2PEG2gEC12)-T-7MeW-K (Ac)-Pen-AEF-2Nal-THP-K (Ac)-N-3Pya-Sar-CONH2,
MeCO-Pen-N-T-7MeW-K (PEG2PEG2gEC14)-Pen-AEF-2Nal-THP-K (Ac)-N-3Pya-Sar-CONH2,
MeCO-Pen-N-T-7MeW-K (Ac)-Pen-AEF-2Nal-THP-K (PEG2PEG2gEC12)-N-3Pya-Sar-CONH2,
MeCO-r-Abu-N-T-7MeW-K (Ac)-aMeC-AEF-2Nal-THP-K (PEG2PEG2gEC12)-N-3Pya-Sar-CONH2,
MeCO-Pen-K (PEG2PEG2gEC14)-T-7MeW-K (Ac)-Pen-AEF-2Nal-THP-E-N-3Pya-Sar-CONH2,
MeCO-Pen-N-T-7MeW-K (PEG2PEG2gEC12)-Pen-AEF-2Nal-THP-E-N-3Pya-Sar-CONH2,
MeCO-r-Abu-N-T-W-K (Ac)-aMeC-AEF-2Nal-THP-K (PEG2PEG2gEC12)-N-3Pya-Sar-CONH2,
MeCO-r-Abu-N-T-W-K (Ac)-aMeC-AEF-2Nal-THP-E-N-3Pya-Sar-K (PEG2PEG2gEC12)-CONH2,
MeCO-r-Abu-N-T-7MeW-K (PEG2PEG2gEC12)-aMeC-AEF-2Nal-THP-E-N-3Pya-Sar-CONH2,
MeCO-r-Abu-N-T-7MeW-K (Ac)-aMeC-AEF-2Nal-THP-E-N-3Pya-Sar-K (PEG2PEG2gEC12)-CONH2,
MeCO-r-Abu-N-T-W-K (PEG2PEG2gEC12)-aMeC-AEF-2Nal-THP-E-N-3Pya-Sar-CONH2,
MeCO-Pen-N-T-7MeW-K (Ac)-Pen-AEF (G)-2Nal-THP-K (PEG2PEG2gEC14)-N-3Pya-Sar-CONH2,
MeCO-Pen-N-T-7MeW-K (Ac)-Pen-AEF-2Nal-THP-E-N-3Pya-Sar-K (PEG2PEG2gEC12)-CONH2,
MeCO-Pen-N-T-7MeW-K (Ac)-Pen-AEF-2Nal-THP-E-N-3Pya-Sar-K (PEG2PEG2gEC14)-CONH2,
MeCO-Pen-K (PEG2PEG2gEC12)-T-7MeW-K (Ac)-Pen-AEF-2Nal-THP-E-N-3Pya-Sar-CONH2,
MeCO-r-Pen-N-T-7MeW-K (Ac)-Pen-AEF-2Nal-THP-E-N-3Pya-Sar-K (PEG2NMePEG2NMegENMeC18Tetrazole)-CONH2,
MeCO-Pen-K (C14)-T-7MeW-K (Ac)-Pen-AEF-2Nal-THP-E-N-3Pya-Sar-CONH2,
MeCO-Pen-N-T-7MeW-K (C14)-Pen-AEF-2Nal-THP-E-N-3Pya-Sar-CONH2,
MeCO-Pen-N-T-7MeW-K (Ac)-Pen-AEF-2Nal-THP-K (C14)-N-3Pya-Sar-CONH2,
MeCO-Pen-K (gEC14)-T-7MeW-K (Ac)-Pen-AEF-2Nal-THP-E-N-3Pya-Sar-CONH2,
MeCO-Pen-N-T-7MeW-K (gEC14)-Pen-AEF-2Nal-THP-E-N-3Pya-Sar-CONH2,
MeCO-Pen-N-T-7MeW-K (Ac)-Pen-AEF-2Nal-THP-K (gEC14)-N-3Pya-Sar-CONH2,
MeCO-Pen-N-T-7MeW-K (Ac)-Pen-AEF-2Nal-THP-K (Ac)-N-3Pya-Sar-K (PEG2PEG2gEC14)-CONH2,
MeCO-Pen-N-T-7MeW-K (Ac)-Pen-AEF-2Nal-THP-E-N-3Pya-Sar-K (gEC14)-CONH2,
MeCO-Pen-N-T-7MeW-K (Ac)-Pen-AEF-2Nal-THP-E-N-3Pya-Sar-K (gEC12)-CONH2,
MeCO-k (PEG2PEG2gEC12)-Pen-N-T-7MeW-K (Ac)-Pen-AEF-2Nal-THP-K (Ac)-N-3Pya-Sar-CONH2,
MeCO-k (PEG2PEG2gEC14)-Pen-N-T-7MeW-K (Ac)-Pen-AEF-2Nal-THP-K (Ac)-N-3Pya-Sar-CONH2,
MeCO-Pen-N-T-7MeW-K (Ac)-Pen-AEF-2Nal-THP-E-N-3Pya-Sar-K (C14)-CONH2,
MeCO-Pen-N-T-7MeW-K (Ac)-Pen-AEF-2Nal-THP-K (Ac)-N-3Pya-Sar-K (PEG2PEG2gEC12)-CONH2,
MeCO-Pen-N-T-7MeW-K (Ac)-Pen-AEF-2Nal-THP-E-N-3Pya-Sar-K (PEG2PEG2gEDProC14)-CONH2,
MeCO-Pen-N-T-7MeW-K (Ac)-Pen-AEF-2Nal-THP-E-N-3Pya-Sar-K (PEG2PEG2C14)-CONH2,
MeCO-Pen-N-T-7MeW-K (Ac)-Pen-AEF-2Nal-THP-E-N-3Pya-Sar-K (GSGSGSGC14)CONH2,
MeCO-Pen-N-T-7MeW-K (Ac)-Pen-AEF-2Nal-THP-E-N-3Pya-Sar-K (PEG2PEG2SP6C14)-CONH2,
MeCO-Pen-N-T-7MeW-K (Ac)-Pen-AEF-2Nal-THP-E-N-3Pya-Sar-K (PEG2C14)-CONH2,
MeCO-Pen-N-T-7MeW-K (Ac)-Pen-AEF-2Nal-THP-E-N-3Pya-Sar-K (PEG2PEG2gESarC14)-CONH2, and
MeCO-Pen-N-T-7MeW-K (Ac)-Pen-AEF-2Nal-THP-E-N-3Pya-Sar-K (PEG2PEG2gEProC14)-CONH2,
PEG2PEG2-gEC16OH-r-Pen-N-T-7MeW-K (Ac)-Pen-AEF-2Nal-THP-K (Ac)-N-3Pya-Sar-CONH2,
PEG2 Palm-Pen-N-T-7MeW-K (Ac)-Pen-AEF-2Nal-THP-E-N-3Pya-Sar-CONH2,
PEG2_Lauryl-Pen-N-T-7MeW-K (Ac)-Pen-AEF-2Nal-THP-E-N-3Pya-Sar-CONH2,
PEG4 Palm-Pen-N-T-7MeW-K (Ac)-Pen-AEF-2Nal-THP-E-N-3Pya-Sar-CONH2,
PEG4_Lauryl-Pen-N-T-7MeW-K (Ac)-Pen-AEF-2Nal-THP-E-N-3Pya-Sar-CONH2,
PEG4_Capryl-Pen-N-T-7MeW-K (Ac)-Pen-AEF-2Nal-THP-E-N-3Pya-Sar-CONH2,
PEG4_Hexyl-Pen-N-T-7MeW-K (Ac)-Pen-AEF-2Nal-THP-E-N-3Pya-Sar-CONH2,
C12gEPEG2PEG2CO-Pen-N-T-7MeW-K (PEG2PEG2gEC12)-Pen-AEF-2Nal-THP-E-N -3Pya-Sar-CONH2,
C14gEPEG2PEG2CO-Pen-N-T-7MeW-K (PEG2PEG2gEC14)-Pen-AEF-2Nal-THP-E-N -3Pya-Sar-CONH2,
C14gEPEG2PEG2CO-k (PEG2PEG2gEC14)-Pen-N-T-7MeW-K (Ac)-Pen-AEF-2Nal-THP -E-N-3Pya-Sar-CONH2,
C12gEPEG2PEG2CO-k (PEG2PEG2gEC12)-Pen-N-T-7MeW-K (Ac)-Pen-AEF-2Nal-THP -E-N-3Pya-Sar-CONH2,
C14gEPEG2PEG2CO-Pen-N-T-7MeW-K (Ac)-Pen-AEF-2Nal-THP-E-N-3Pya-Sar-K (PEG2PEG2gEC14)-CONH2,
C12gEPEG2PEG2CO-Pen-N-T-7MeW-K (Ac)-Pen-AEF-2Nal-THP-E-N-3Pya-Sar-CONH2,
C14gEPEG2PEG2CO-Pen-N-T-7MeW-K (Ac)-Pen-AEF-2Nal-THP-E-N-3Pya-Sar-CONH2,
C12gEPEG2PEG2CO-Pen-K (PEG2PEG2gEC12)-T-7MeW-K (Ac)-Pen-AEF-2Nal-THP -E-N-3Pya-Sar-CONH2,
C14gEPEG2PEG2CO-Pen-K (PEG2PEG2gEC14)-T-7MeW-K (Ac)-Pen-AEF-2Nal-THP -E-N-3Pya-Sar-CONH2,
C12gEPEG2PEG2CO-Pen-N-T-7MeW-K (Ac)-Pen-AEF-2Nal-THP-K (PEG2PEG2gEC12)-N-3Pya-Sar-CONH2,
C14gEPEG2PEG2CO-Pen-N-T-7MeW-K (Ac)-Pen-AEF-2Nal-THP-K (PEG2PEG2gEC14)-N-3Pya-Sar-CONH2,
C12gEPEG2PEG2CO-r-Pen-N-T-7MeW-K (Ac)-Pen-AEF-2Nal-THP-E-N-3Pya-Sar-CONH2,
C12gEPEG2PEG2CO-r-Pen-N-T-7MeW-K (Ac)-Pen-AEF-2Nal-THP-E-N-3Pya-Sar-K (PEG2PEG2gEC12)-CONH2,
C12gEPEG2PEG2CO-r-Pen-N-T-7MeW-K (Ac)-Pen-AEF-2Nal-THP-K (PEG2PEG2gEC12)-N-3Pya-Sar-CONH2,
C12gEPEG2PEG2CO-Pen-N-T-7MeW-K (Ac)-Pen-AEF (G)-2Nal-THP-E-N-3Pya-Sar-CONH2,
C12gEPEG2PEG2CO-r-Pen-N-T-7MeW-K (PEG2PEG2gEC12)-Pen-AEF-2Nal-THP-E-N -3Pya-Sar-CONH2,
C12gEPEG2PEG2CO-Pen-N-T-7MeW-K (Ac)-Pen-AEF-2Nal-THP-K (Ac)-N-3Pya-Sar-CONH2, and
C14gEPEG2PEG2CO-Pen-N-T-7MeW-K (Ac)-Pen-AEF-2Nal-THP-K (Ac)-N-3Pya-Sar-CONH2.

2. The polypeptide of claim 1, comprising the amino acid sequence: 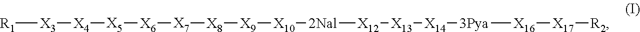
or a pharmaceutically acceptable salt thereof, wherein:
R₁ is MeCO, Z_{peg}, Z_{lipid}, succiniccarn, 5cpaCO, or AEEP-Z_{lipid};
X₃ is R, K—Z_{lipid}, Dab-Z_{lipid}, NMeK—Z_{lipid}, or absent;
X₄ is 4AminoPro, Abu, aG, aMeC, C, Dap, Pen, Pen(oXyl), Pen(mXyl), Pen(pXyl), or Pra;
X₅ is N, N(Nme₂), K(d), K—Z_{lipid}, Dab-Z_{lipid}, or NmeK-Z_{lipid};
X₆ is T, K—Z_{lipid}, NmeK-Z_{lipid}, or Dab-Z_{lipid};
X₇ is 7MeW, W, or absent;
X₈ is K(Ac), K(d), K(NmeAc), Q, K—Z_{lipid}, K—Z_{peg}, NmeK-Z_{lipid}, Dab-Z_{lipid}, Dab(Nmecarn), or Dab-Z_{peg};
X₉ is aMeC, aG, C, D, E, hE, Pen, Dap, or Dap(N3);
X₁₀ is AEF, TMAPF, AEF(d), TMAPF-Z_{lipid}, APEG3F, or AEF-Z_{lipid};
X₁₂ is THP, K—Z_{lipid}, Dab-Z_{lipid}, or NmeK-Z_{lipid};
X₁₃ is E, K(Ac), K(d), K—Z_{peg}, K(NmeAc), Dab(Nmecarn), E(Oall), K—Z_{lipid}, Dab-Z_{lipid}, NmeK-Z_{lipid}, or absent;
X₁₄ is N, K—Z_{lipid}, NmeK-Z_{lipid}, or Dab-Z_{lipid}
X₁₆ is Sar, NmeK(d), K—Z_{lipid}, Dab-Z_{lipid}, or NmeK-Z_{lipid};
X₁₇ is K—Z_{lipid}, Dab-Z_{lipid}, NmeK-Z_{lipid}, or absent;
R₂ is CONH2, CONMe2, CONH—Z_{peg}, or CO—Z_{lipid};
Z_{peg}, independently for each occurrence, is a polyethylene glycol chain;
Z_{lipid}, independently for each occurrence, is a lipophilic substituent;
wherein the peptide comprises a linkage between the residues at X₄ and X₉; and
wherein the polypeptide comprises at least one lipophilic substituent;
provided that the polypeptide is not selected from the group consisting of:
MeCO-r-Pen-N-T-7MeW—K(Ac)-Pen-AEF-2Nal-THP-E-N-3Pya-Sar-K(PEG2PEG2gEC18OH)—CONH2,
MeCO-r-Pen-N-T-7MeW—K(Ac)-Pen-AEF-2Nal-THP-E-N-3Pya-NmeK(PEG2PEG2gEC18OH)—CONH2,
MeCO-Pen-N-T-7MeW—K(Ac)-Pen-AEF-2Nal-THP-K(PEG2PEG2gEC18OH)—N-3Pya-Sar-CONH2,
MeCO-k(PEG2PEG2gEC18OH)-Pen-N-T-7MeW—K(Ac)-Pen-AEF-2Nal-THP-K(Ac)—N-3Pya-Sar-CONH2,
MeCO-r-Pen-N-T-7MeW—K(Ac)-Pen-AEF(PEG2PEG2gEC18OH)-2Nal-THP-K(Ac)—N-3Pya-Sar-CONH2,
MeCO-k(PEG2PEG2SP6gEC18OH)-Pen-N-T-7MeW—K(Ac)-Pen-AEF-2Nal-THP-K(Ac)—N-3Pya-Sar-CONH2,
MeCO-k(PEG2PEG2gEmXOH)-Pen-N-T-7MeW—K(Ac)-Pen-AEF-2Nal-THP-K(PEG2PEG2gEmXOH)—N-3Pya-Sar-CONH2,
MeCO-k(PEG2PEG2gEC16OH)-Pen-N-T-7MeW—K(Ac)-Pen-AEF-2Nal-THP-K(gEC16)-N-3Pya-Sar-CONH2,
HOC18gEPEG2PEG2-r-Pen-N-T-7MeW—K(Ac)-Pen-AEF-2Nal-THP-K(Ac)—N-3Pya-Sar-CONH2,
HOC18gEPEG2PEG2-r-Pen-N-T-7MeW—K(Ac)-Pen-AEF-2Nal-THP-K(PEG2PEG2gEC18OH)—N-3Pya-Sar-CONH2,
MeCO-k(PEG2PEG2gEDab(mXOH)2)-Pen-N-T-7MeW—K(Ac)-Pen-AEF-2Nal-THP-K(Ac)—N-3Pya-Sar-CONH2,
MeCO-k(PEG2PEG2gE(c)C18OH)-Pen-N-T-7MeW—K(Ac)-Pen-AEF-2Nal-THP-K(Ac)—N-3Pya-Sar-CONH2,
MeCO-r-Pen-N-T-7MeW—K(Ac)-Pen-AEF-2Nal-THP-E-N-3Pya-Sar-K(PEG2PEG2gEC16OH)—CONH2,
MeCO-r-Pen-N-T-7MeW—K(Ac)-Pen-AEF-2Nal-THP-E-N-3Pya-K(PEG2PEG2gEC18OH)—CONH2,
MeCO-Pen-N-T-7MeW—K(Ac)-Pen-AEF-2Nal-THP-K(PEG2PEG2gEC16OH)—N-3Pya-Sar-CONH2,
MeCO-Pen-K(PEG2PEG2gEC18OH)-T-7MeW—K(Ac)-Pen-AEF-2Nal-THP-E-N-3Pya-Sar-CONH2,
MeCO-r-Pen-K(PEG2PEG2gEC16OH)-T-7MeW—K(Ac)-Pen-AEF-2Nal-THP-E-N-3Pya-Sar-CONH2,
MeCO-r-Pen-N-T-7MeW—K(Ac)-Pen-AEF-2Nal-THP-E-N-3Pya-NMeK(PEG2PEG2gEC16OH)—CONH2,
MeCO-r-Pen-N-T-7MeW—K(Ac)-Pen-AEF-2Nal-THP-E-N-3Pya-K(PEG2PEG2gEC16OH)—CONH2,
MeCO-k(PEG2PEG2gEC16OH)-Pen-N-T-7MeW—K(Ac)-Pen-AEF-2Nal-THP-K(Ac)—N-3Pya-Sar-CONH2,
MeCO-r-Pen-N-T-7MeW—K(Ac)-Pen-AEF(PEG2PEG2gEC16OH)-2Nal-THP-K(Ac)—N-3Pya-Sar-CONH2,
MeCO-r-Pen-N-T-7MeW—K(Ac)-Pen-AEF-2Nal-THP-K(PEG2PEG2gEC18OH)—N-3Pya-Sar-CONH2,
MeCO-r-Pen-N-T-7MeW—K(Ac)-Pen-AEF-2Nal-THP-K(PEG2PEG2gEC16OH)—N-3Pya-Sar-CONH2,
MeCO-Pen-N-T-7MeW—K(Ac)-Pen-AEF-2Nal-THP-K(gEC16OH)—N-3Pya-Sar-CONH2,
MeCO-Pen-N-T-7MeW—K(Ac)-Pen-AEF-2Nal-THP-K(gEC18OH)—N-3Pya-Sar-CONH2,
MeCO-Pen-N-T-7MeW—K(Ac)-Pen-AEF-2Nal-THP-K(gEC18)-N-3Pya-Sar-CONH2,
MeCO-Pen-N-T-7MeW—K(Ac)-Pen-AEF-2Nal-THP-K(PEG6gEC16OH)—N-3Pya-Sar-CONH2,
MeCO-Pen-N-T-7MeW—K(Ac)-Pen-AEF-2Nal-THP-K(PEG6gEC18OH)—N-3Pya-Sar-CONH2,
MeCO-Pen-N-T-7MeW—K(Ac)-Pen-AEF-2Nal-THP-K(PEG2gEC16OH)—N-3Pya-Sar-CONH2,
MeCO-Pen-N-T-7MeW—K(Ac)-Pen-AEF-2Nal-THP-K(PEG2gEC18OH)—N-3Pya-Sar-CONH2,
MeCO-Pen-N-T-7MeW—K(Ac)-Pen-AEF-2Nal-THP-K(PEG2PEG2gEC20OH)—N-3Pya-Sar-CONH2,
HOC16gEPEG2PEG2-r-Pen-N-T-7MeW—K(Ac)-Pen-AEF-2Nal-THP-K(PEG2PEG2gEC16OH)—N-3Pya-Sar-CONH2,
MeCO-Pen-N-T-7MeW—K(Ac)-Pen-AEF-2Nal-THP-K(PEG2PEG6gEC16OH)—N-3Pya-Sar-CONH2,
MeCO-Pen-N-T-7MeW—K(Ac)-Pen-AEF-2Nal-THP-K(PEG2PEG6gEC18OH)—N-3Pya-Sar-CONH2,
MeCO-Pen-N-T-7MeW—K(Ac)-Pen-AEF-2Nal-THP-K(PEG2PEG2C16OH)—N-3Pya-Sar-CONH2,
MeCO-Pen-N-T-7MeW—K(Ac)-Pen-AEF-2Nal-THP-K(PEG2PEG2C18OH)—N-3Pya-Sar-CONH2,
MeCO-Pen-N-T-7MeW—K(Ac)-Pen-AEF-2Nal-THP-K(PEG6PEG6gEC16OH)—N-3Pya-Sar-CONH2,
MeCO-Pen-N-T-7MeW—K(Ac)-Pen-AEF-2Nal-THP-K(PEG6PEG6gEC18OH)—N-3Pya-Sar-CONH2,
MeCO-Pen-N-T-7MeW—K(Ac)-Pen-AEF-2Nal-THP-K(PEG24-gEC16OH)—N-3Pya-Sar-CONH2,
MeCO-Pen-N-T-7MeW—K(Ac)-Pen-AEF-2Nal-THP-K(PEG24-gEC18OH)—N-3Pya-Sar-CONH2,
MeCO-Pen-N-T-7MeW—K(Ac)-Pen-AEF-2Nal-THP-K(PEG24-C18OH)—N-3Pya-Sar-CONH2,
MeCO-k(gEC18OH)-Pen-N-T-7MeW—K(Ac)-Pen-AEF-2Nal-THP-K(Ac)—N-3Pya-Sar-CONH2,
MeCO-k(PEG2gEC18OH)-Pen-N-T-7MeW—K(Ac)-Pen-AEF-2Nal-THP-K(Ac)—N-3Pya-Sar-CONH2,
MeCO-r-Pen-K(PEG2PEG2gEC18OH)-T-7MeW—K(Ac)-Pen-AEF-2Nal-THP-K(Ac)—N-3Pya-Sar-CONH2,
MeCO-r-Pen-K(gEC18OH)-T-7MeW—K(Ac)-Pen-AEF-2Nal-THP-K(Ac)—N-3Pya-Sar-CONH2,
MeCO-r-Pen-K(PEG2gEC18OH)-T-7MeW—K(Ac)-Pen-AEF-2Nal-THP-K(Ac)—N-3Pya-Sar-CONH2,
MeCO-r-Pen-K(PEG2PEG2C18OH)-T-7MeW—K(Ac)-Pen-AEF-2Nal-THP-K(Ac)—N-3Pya-Sar-CONH2,
MeCO-r-Pen-N-T-7MeW—K(Ac)-Pen-AEF-2Nal-THP-K(Ac)—N-3Pya-NMeK(PEG2PEG2gEC18OH)—CONH2,
MeCO-r-Pen-N-T-7MeW—K(Ac)-Pen-AEF-2Nal-THP-K(PEG2PEG2-DgEC18OH)—N-3Pya-Sar-CONH2,
MeCO-r-Pen-N-T-7MeW—K(Ac)-Pen-AEF-2Nal-THP-K(PEG2PEG2-pC18OH)—N-3Pya-Sar-CONH2,
MeCO-r-Pen-N-T-7MeW—K(Ac)-Pen-AEF-2Nal-THP-K(PEG2PEG2gETrxC18OH)—N-3Pya-Sar-CONH2,
MeCO-r-Pen-N-T-7MeW—K(Ac)-Pen-AEF-2Nal-THP-K(PEG2PEG2gETrxC20OH)—N-MeCO-k(PEG6gEC18OH)-Pen-N-T-7MeW—K(Ac)-Pen-AEF-2Nal-THP-K(Ac)—N-3Pya-Sar-CONH2,
MeCO-k(PEG2PEG6gEC18OH)-Pen-N-T-7MeW—K(Ac)-Pen-AEF-2Nal-THP-K(Ac)—N-3Pya-Sar-CONH2,
MeCO-k(PEG2PEG2C18OH)-Pen-N-T-7MeW—K(Ac)-Pen-AEF-2Nal-THP-K(Ac)—N-3Pya-Sar-CONH2,
MeCO-r-Pen-K(PEG6gEC18OH)-T-7MeW—K(Ac)-Pen-AEF-2Nal-THP-K(Ac)—N-3Pya-Sar-CONH2,
MeCO-r-Pen-K(PEG2PEG6gEC18OH)-T-7MeW—K(Ac)-Pen-AEF-2Nal-THP-K(Ac)—N-3Pya-Sar-CONH2,
MeCO-r-Pen-N-T-7MeW—K(Ac)-Pen-AEF-2Nal-THP-K(PEG2PEG2-pppC18OH)—N-3Pya-Sar-CONH2,
MeCO-k(gEC16)-Pen-N-T-7MeW—K(Ac)-Pen-AEF-2Nal-THP-K(Ac)—N-3Pya-Sar-CONH2,
MeCO-k(gEC18)-Pen-N-T-7MeW—K(Ac)-Pen-AEF-2Nal-THP-K(Ac)—N-3Pya-Sar-CONH2,
MeCO-r-Pen-K(gEC16)-T-7MeW—K(Ac)-Pen-AEF-2Nal-THP-K(Ac)—N-3Pya-Sar-CONH2,
MeCO-r-Pen-K(gEC18)-T-7MeW—K(Ac)-Pen-AEF-2Nal-THP-K(Ac)—N-3Pya-Sar-CONH2,
MeCO-k(PEG2PEG2gETrxC18OH)-Pen-N-T-7MeW—K(Ac)-Pen-AEF-2Nal-THP-K(Ac)—N-3Pya-Sar-CONH2,
MeCO-k(PEG2PEG2gETrxC20OH)-Pen-N-T-7MeW—K(Ac)-Pen-AEF-2Nal-THP-K(Ac)—N-3Pya-Sar-CONH2,
MeCO-Pen-K(PEG2PEG2gETrxC18OH)-T-7MeW—K(Ac)-Pen-AEF-2Nal-THP-K(Ac)—N-3Pya-Sar-CONH2,
MeCO-Pen-K(PEG2PEG2gETrxC20OH)-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-K(gEC16)-N-3Pya-Sar-CONH2,
MeCO-k(PEG2PEG2gEC16OH)-Pen-K(PEG2PEG2gEC16OH)-T-7MeW—K(Ac)-Pen-AEF-2Nal-THP-K(Ac)—N-3Pya-Sar-CONH2,
MeCO-k(PEG2PEG2gEC18OH)-Pen-K(PEG2PEG2gEC18OH)-T-7MeW—K(Ac)-Pen-AEF-2Nal-THP-K(Ac)—N-3Pya-Sar-CONH2,
MeCO-k(PEG2PEG2gEC20OH)-Pen-K(PEG2PEG2gEC20OH)-T-7MeW—K(Ac)-Pen-AEF-2Nal-THP-K(Ac)—N-3Pya-Sar-CONH2,
MeCO-k(gEC16)-Pen-K(gEC16)-T-7MeW—K(Ac)-Pen-AEF-2Nal-THP-K(Ac)—N-3Pya-Sar-CONH2,
MeCO-k(gEC18)-Pen-K(gEC18)-T-7MeW—K(Ac)-Pen-AEF-2Nal-THP-K(Ac)—N-3Pya-Sar-CONH2,
MeCO-k(PEG2PEG2gEC16)-Pen-N-T-7MeW—K(Ac)-Pen-AEF-2Nal-THP-K(Ac)—N-3Pya-Sar-CONH2,
MeCO-k(PEG2PEG2gEC18)-Pen-N-T-7MeW—K(Ac)-Pen-AEF-2Nal-THP-K(Ac)—N-3Pya-Sar-CONH2,
MeCO-Pen-K(PEG2PEG2gEC16)-T-7MeW—K(Ac)-Pen-AEF-2Nal-THP-K(Ac)—N-3Pya-Sar-CONH2,
MeCO-Pen-K(PEG2PEG2gEC18)-T-7MeW—K(Ac)-Pen-AEF-2Nal-THP-K(Ac)—N-3Pya-Sar-CONH2,
MeCO-Pen-K(1PEG21PEG2IsoGluC16Diacid)-T-7MeW—K(Ac)-Pen-AEF-2Nal-TIP—K(Ac)—N-3Pya-Sar-CONH2,
MeCO-r-Pen-N-T-7MeW—K(PEG2PEG2gEC18OH)-Pen-AEF-2Nal-THP-K(Ac)—N-3Pya-Sar-CONH2,
MeCO-Pen-N-T-7MeW—K(Ac)-Pen-AEF-2Nal-THP-K(gEC20OH)—N-3Pya-Sar-CONH2,
MeCO-Pen-N-T-7MeW—K(Ac)-Pen-AEF-2Nal-THP-K(PEG2PEG2-TrxgEC18OH)—N-3Pya-Sar-CONH2,
MeCO-Pen-N-T-7MeW—K(Ac)-Pen-AEF-2Nal-THP-K(PEG2PEG2-TrxgETrxC20OH)—N-3Pya-Sar-CONH2,
MeCO-Pen-N-T-7MeW—K(Ac)-Pen-AEF-2Nal-THP-K(PEG2PEG2-TrxgEC20OH)—N-3Pya-Sar-CONH2,
MeCO-k(PEG2PEG2gEC10OH)-Pen-N-T-7MeW—K(Ac)-Pen-AEF-2Nal-THP-K(gEC16)-N-3Pya-Sar-CONH2,
MeCO-k(PEG2PEG2gEC18OH)-Pen-K(gEC16)-T-7MeW—K(Ac)-Pen-AEF-2Nal-THP-K(Ac)—N-3Pya-Sar-CONH2,
MeCO-r-Pen-K(PEG2PEG2pgEC18OH)-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-K(PEG2PEG2gEmXOH)—N-3Pya-Sar-CONH2,
MeCO-k(PEG2PEG2gEC18OH)-Pen-N-T-7MeW—K(Ac)-Pen-AEF-2Nal-THP-K(gEC16)-N-3Pya-Sar-CONH2,
MeCO-r-Pen-K(PEG2PEG2pppgEC18OH)-T-7MeW—K(Ac)-Pen-AEF-2Nal-THP-K(Ac)—N-3Pya-Sar-CONH2,
MeCO-k(PEG2PEG2PgEC18OH)-Pen-N-T-7MeW—K(Ac)-Pen-AEF-2Nal-THP-K(Ac)—N-3Pya-Sar-CONH2,
MeCO-k(PEG2PEG2PPPgEC18OH)-Pen-N-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-K(PEG2PEG2PgEC18OH)—N-3Pya-Sar-CONH2,
MeCO-Pen-N-T-7MeW—K(Ac)-Pen-AEF-2Nal-THP-K(PEG2PEG2PPPgEC18OH)—N-3Pya-Sar-CONH2,
MeCO-k(PEG2PEG2-TrxgEC18OH)-Pen-N-T-7MeW—K(Ac)-Pen-AEF-2Nal-THP-K(Ac)—N-3Pya-Sar-CONH2,
MeCO-k(PEG2PEG2gEmXOH)-Pen-N-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-K(PEG2PEG2gEpXOH)—N-3Pya-Sar-CONH2,
MeCO-k(PEG2PEG2gEpXOH)-Pen-N-T-7MeW—K(Ac)-Pen-AEF-2Nal-THP-K(PEG2PEG2gEpXOH)—N-3Pya-Sar-CONH2,
MeCO-k(PEG2PEG2gEmXOH)-Pen-N-T-7MeW—K(Ac)-Pen-AEF-2Nal-THP-K(gEC16)-N-3Pya-Sar-CONH2,
MeCO-Pen-N-T-7MeW—K(Ac)-Pen-AEF-2Nal-THP-K(DAP(C16OH)2)-N-3Pya-Sar-CONH2,
MeCO-k(DAP(C16OH)2)-Pen-N-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-K(PEG2PEG2gEDAP(C16OH)2)-N-3Pya-Sar-CONH2,
MeCO-k(PEG2PEG2gEDAP(C16OH)2)-Pen-N-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-K(PEG2PEG2Sp6gEC18OH)—N-3Pya-Sar-CONH2,
MeCO-Pen-N-T-7MeW—K(Ac)-Pen-AEF-2Nal-THP-K(PEG2Sp6PEG2gEC18OH)—N-3Pya-Sar-CONH2,
MeCO-k(PEG2PEG2gEC16OH)-Pen-N-T-7MeW—K(Ac)-Pen-AEF-2Nal-THP-K(gEC18)-N-3Pya-Sar-CONH2,
MeCO-k(PEG2PEG2gEC18OH)-Pen-N-T-7MeW—K(Ac)-Pen-AEF-2Nal-THP-K(gEC18)-N-3Pya-Sar-CONH2,
MeCO-Pen-K(1PEG21PEG2IsoGluC18Diacid)-T-7MeW—K(Ac)-Pen-AEF-2Nal-THP-K(Ac)—N-3Pya-Sar-CONH2,
MeCO-Pen-K(PEG2PEG2gEC18OH)-T-7MeW—K(Ac)-Pen-AEF-2Nal-THP-K(PEG2PEG2gESp6C18OH)—N-3Pya-Sar-CONH2,
MeCO-Pen-K(PEG2PEG2gEC18OH)-T-7MeW—K(Ac)-Pen-AEF-2Nal-THP-K(Sp6PEG2PEG2gEC18OH)—N-3Pya-Sar-CONH2,
MeCO-k(gEC16)-Pen-N-T-7MeW—K(Ac)-Pen-AEF-2Nal-THP-K(gEC18)-N-3Pya-Sar-CONH2,
MeCO-k(gEC18)-Pen-N-T-7MeW—K(Ac)-Pen-AEF-2Nal-THP-K(gEC18)-N-3Pya-Sar-CONH2,
MeCO-k(PEG2PEG2gEC10OH)-Pen-N-T-7MeW—K(Ac)-Pen-AEF-2Nal-THP-K(gEC18)-N-3Pya-Sar-CONH2,
MeCO-Pen-K(PEG2PEG2gEC18OH)-T-7MeW—K(Ac)-Pen-AEF-2Nal-THP-K(PEG2PEG2gETrxC20OH)—N-3Pya-Sar-CONH2,
MeCO-k(PEG2PEG2gESp6C18OH)-Pen-N-T-7MeW—K(Ac)-Pen-AEF-2Nal-THP-K(Ac)—N-3Pya-Sar-CONH2,
MeCO-k(PEG2Sp6PEG2gEC18OH)-Pen-N-T-7MeW—K(Ac)-Pen-AEF-2Nal-THP-K(Ac)—N-3Pya-Sar-CONH2,
MeCO-k(Sp6PEG2PEG2gEC18OH)-Pen-N-T-7MeW—K(Ac)-Pen-AEF-2Nal-THP-K(Ac)—N-3Pya-Sar-CONH2,
MeCO—K[PEG2PEG2-gEDAP(pXOH)2]-Pen-N-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-K[PEG2PEG2-gEDAP-(mXOH)2]—N-3Pya-Sar-CONH2,
MeCO-k(GolAC16)-Pen-N-T-7MeW—K(Ac)-Pen-AEF-2Nal-THP-K(Ac)—N-3Pya-Sar-CONH2,
MeCO-k(GolAC16OH)-Pen-N-T-7MeW—K(Ac)-Pen-AEF-2Nal-THP-K(Ac)—N-3Pya-Sar-CONH2,
MeCO-k(GolAC18OH)-Pen-N-T-7MeW—K(Ac)-Pen-AEF-2Nal-THP-K(Ac)—N-3Pya-Sar-CONH2,
MeCO-k(PEG2PEG2 GolAC18OH)-Pen-N-T-7MeW—K(Ac)-Pen-AEF-2Nal-THP-K(Ac)—N-3Pya-Sar-CONH2,
MeCO-k(PEG2PEG2-C18GolB)-Pen-N-T-7MeW—K(Ac)-Pen-AEF-2Nal-THP-K(Ac)—N-3Pya-Sar-CONH2,
MeCO-k(PEG2PEG2gE(C)C18OH)-Pen-N-T-7MeW—K(Ac)-Pen-AEF-2Nal-THP-K(Ac)—N-3Pya-Sar-CONH2,
MeCO-k(PEG2PEG2gEC18OH(C))-Pen-N-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-E-N-3Pya-Sar-k(PEG12_IsoGlu_C18_Diacid)-CONH2,
MeCO-Pen-N-T-7MeW—K(Ac)-Pen-AEF-2Nal-THP-E-N-3Pya-Sar-k(Peg4_IsoGlu_Palm)-CONH2,
MeCO-Pen-N-T-7MeW—K(Ac)-Pen-AEF-2Nal-THP-E-N-3Pya-Sar-k(IsoGlu_Palm)-CONH2,
MeCO-Pen-N-T-7MeW—K(Ac)-Pen-AEF-2Nal-TIP-E-N-3Pya-Sar-k(PEG12_C18_Diacid)-CONH2,
MeCO-Pen-N-T-7MeW—K(Ac)-Pen-AEF-2Nal-THP-E-N-3Pya-Sar-k(Peg4_IsoGlu_C18_Diacid)-CONH2,
MeCO-Pen-N-T-7MeW—K(Ac)-Pen-AEF-2Nal-THP-E-N-3Pya-Sar-k(PEG12_IsoGlu_Palm)-CONH2,
MeCO-Pen-N-T-7MeW—K(Ac)-Pen-AEF-2Nal-THP-E-N-3Pya-Sar-k(Peg4_C18_Diacid)-CONH2,
MeCO-Pen-N-T-7MeW—K(Ac)-Pen-AEF-2Nal-THP-E-N-3Pya-Sar-k(IsoGlu_C18_Diacid)-CONH2,
MeCO-r-Pen-K(1PEG21PEG2IsoGluC16Diacid)-T-7MeW—K(Ac)-Pen-AEF-2Nal-THP-E-N-3Pya-Sar-CONH2,
MeCO-r-Pen-K(1PEG21PEG2IsoGluC18Diacid)-T-7MeW—K(Ac)-Pen-AEF-2Nal-TIP-E-N-3Pya-Sar-CONH2,
MeCO-Pen-N-T-7MeW—K(Ac)-Pen-AEF-2Nal-TIP-K(1PEG21PEG2IsoGluC16Diacid)-N-3Pya-Sar-CONH2,
MeCO-Pen-N-T-7MeW—K(Ac)-Pen-AEF-2Nal-TIP-K(1PEG21PEG2IsoGluC18Diacid)-N-3Pya-Sar-CONH2,
MeCO-r-Pen-N-T-7MeW—K(Ac)-Pen-AEF-2Nal-THP-E-N-3Pya-K(1PEG21PEG2IsoGluC18Diacid)-CONH2,
MeCO-r-Pen-N-T-7MeW—K(Ac)-Pen-AEF-2Nal-THP-E-N-3Pya-Sar-K(1PEG21PEG2IsoGluC16Diacid)-CONH2,
MeCO-r-Pen-N-T-7MeW—K(Ac)-Pen-AEF-2Nal-THP-E-N-3Pya-Sar-K(1PEG21PEG2IsoGluC18Diacid)-CONH2,
HOC10gEPEG2COPEG2CO-r-Pen-N-T-7MeW—K(Ac)-Pen-AEF-2Nal-THP-K(PEG2PEG2gEC10OH)—N-3Pya-Sar-CONH2,
MeCO-Pen-N-T-7MeW—K(Ac)-Pen-AEF-2Nal-TIP-K(PEG2PEG2gEC10OH)—N-3Pya-Sar-CONH2,
MeCO-k(PEG2PEG2gEC10OH)-Pen-N-T-7MeW—K(Ac)-Pen-AEF-2Nal-THP-K(Ac)—N-3Pya-Sar-CONH2,
MeCO-Pen-K(PEG2PEG2gEC10OH)-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-K(gEC10OH)—N-3Pya-Sar-CONH2,
MeCO-k(PEG2PEG2gEC10OH)-Pen-K(PEG2PEG2gEC10OH)-T-7MeW—K(Ac)-Pen-AEF-2Nal-THP-K(Ac)—N-3Pya-Sar-CONH2,
HOC16gEPEG2PEG2orn(2)-r-Pen-N-T-7MeW—K(Ac)-Pen-AEF-2Nal-THP-E(2)-N-3Pya-Sar-CONH2,
MeCO-k(PEG2PEG2gEC12OH(c))-Pen-N-T-7MeW—K(Ac)-Pen-AEF-2Nal-THP-K(Ac)—N-3Pya-Sar-CONH2,
MeCO-k(PEG2PEG2gE(C)C₁₂)-Pen-N-T-7MeW—K(Ac)-Pen-AEF-2Nal-THP-K(Ac)—N-3Pya-Sar-CONH2,
MeCO-r-Pen-N-T-7MeW—K(Ac)-Pen-AEF-2Nal-THP-K(FITCPEG4)-N-3Pya-Sar-K(PEG2PEG2gEC18OH)—CONH2,
succiniccarn-k(PEG2PEG2gEC18OH)-Pen-N-T-7MeW—K(Ac)-Pen-AEF-2Nal-THP-E-N-3Pya-Sar-CONH2,
MeCO-r-Pen-K(PEG2PEG2gEC18OH)-T-7MeW—K(Ac)-Pen-dAEF-2Nal-THP-E-N-3Pya-Sar-CONH2,
MeCO-Pen-N-T-W—K(PEG2PEG2gEC20OH)-Pen-AEF-2Nal-THP-K(Ac)—N-3Pya-Sar-CONH2,
MeCO-Pen-K(PEG2PEG2gEC20OH)-T-W—K(Ac)-Pen-AEF-2Nal-THP-K(Ac)—N-3Pya-Sar-CONH2,
MeCO—K(PEG2PEG2gEC20OH)-Pen-N-T-W—K(Ac)-Pen-AEF-2Nal-THP-K(Ac)—N-3Pya-Sar-CONH2,
MeCO-Pen-N-T-W—K(Ac)-Pen-AEF-2Nal-THP-K(Ac)—N-3Pya-Sar-K(PEG2PEG2gEC20OH)—CONH2,
MeCO-r-Pen-N-T-7MeW—K(PEG2PEG2gEC20OH)-Pen-AEF-2Nal-THP-E-N-3Pya-Sar-CONH2,
cPEG3aCO-r-Pen-N-T-7MeW—K(PEG2PEG2gEC20OH)-Pen-AEF-2Nal-THP-E-N-3Pya-Sar-CONH2,
cPEG3aCO-r-Pen-K(PEG2PEG2gEC20OH)-T-7MeW—K(Ac)-Pen-AEF-2Nal-THP-E-N-3Pya-Sar-CONH2,
MeCO-r-Pen-N-T-7MeW—K(Ac)-Pen-AEF-2Nal-THP-E-N-3Pya-Sar-K(PEG2PEG2gEC20OH)—CONH2,
MeCO-r-Abu-N-T-7MeW—K(Ac)-aMeC-AEF-2Nal-THP-E-N-3Pya-Sar-K(PEG2PEG2gEC18OH)—CONH2,
MeCO-r-Abu-N-T-7MeW—K(Ac)-Pen-AEF-2Nal-THP-E-N-3Pya-Sar-K(PEG2PEG2gEC18OH)—CONH2,
MeCO-Pen-N-T-7MeW—K(PEG2PEG2gEC12)-Pen-AEF-2Nal-THP-K(PEG2PEG2gEC12)-N-3Pya-Sar-CONH2,
MeCO-Pen-N-T-7MeW—K(PEG2PEG2gEC14)-Pen-AEF-2Nal-THP-K(PEG2PEG2gEC14)-N-3Pya-Sar-CONH2,
MeCO-k(PEG2PEG2gEC12)-Pen-N-T-7MeW—K(Ac)-Pen-AEF-2Nal-THP-E-N-3Pya-Sar-CONH2,
MeCO-k(PEG2PEG2gEC14)-Pen-N-T-7MeW—K(Ac)-Pen-AEF-2Nal-THP-E-N-3Pya-Sar-CONH2,
MeCO-r-Pen-N-T-7MeW—K(PEG2PEG2gEC12)-Pen-AEF-2Nal-THP-E-N-3Pya-Sar-K(PEG2PEG2gEC12)-CONH2,
MeCO-r-Pen-N-T-7MeW—K(PEG2PEG2gEC12)-Pen-AEF-2Nal-THP-K(PEG2PEG2gEC12)-N-3Pya-Sar-CONH2,
MeCO-r-Pen-N-T-7MeW—K(Ac)-Pen-AEF-2Nal-THP-K(PEG2PEG2gEC12)-N-3Pya-Sar-CONH2,
MeCO-r-Pen-N-T-7MeW—K(PEG2PEG2gEC12)-Pen-AEF-2Nal-THP-E-N-3Pya-Sar-CONH2,
MeCO-r-Pen-N-T-7MeW—K(Ac)-Pen-AEF-2Nal-THP-E-N-3Pya-Sar-K(PEG2PEG2gEC12)-CONH2,
cPEG3aCO-k(PEG2PEG2PEG2PEG2gEC12)-Pen-N(N(Me)₂)-T-7MeW—K(Ac)-Pen-TMApF-2Nal-THP-K(nMeAc)—N-3Pya-NMeK(PEG2PEG2PEG2PEG2gEC12)-CONH2,
MeCO-Pen-N-T-7MeW—K(PEG2PEG2gEC14)-Pen-AEF-2Nal-THP-E-N-3Pya-Sar-MeCO-Pen-K(PEG2PEG2gEC14)-T-7MeW—K(Ac)-Pen-AEF-2Nal-THP-K(Ac)—N-3Pya-Sar-CONH2,
MeCO-Pen-N-T-7MeW—K(PEG2PEG2gEC12)-Pen-AEF-2Nal-THP-K(Ac)—N-3Pya-Sar-CONH2,
MeCO-Pen-N-T-7MeW—K(Ac)-Pen-AEF-2Nal-THP-K(PEG2PEG2gEC14)-N-3Pya-Sar-CONH2,
MeCO-Pen-K(PEG2PEG2gEC12)-T-7MeW—K(Ac)-Pen-AEF-2Nal-THP-K(Ac)—N-3Pya-Sar-CONH2,
MeCO-Pen-N-T-7MeW—K(PEG2PEG2gEC14)-Pen-AEF-2Nal-THP-K(Ac)—N-3Pya-Sar-CONH2,
MeCO-Pen-N-T-7MeW—K(Ac)-Pen-AEF-2Nal-THP-K(PEG2PEG2gEC12)-N-3Pya-Sar-CONH2,
MeCO-r-Abu-N-T-7MeW—K(Ac)-aMeC-AEF-2Nal-THP-K(PEG2PEG2gEC12)-N-3Pya-Sar-CONH2,
MeCO-Pen-K(PEG2PEG2gEC14)-T-7MeW—K(Ac)-Pen-AEF-2Nal-THP-E-N-3Pya-Sar-CONH2,
MeCO-Pen-N-T-7MeW—K(PEG2PEG2gEC12)-Pen-AEF-2Nal-THP-E-N-3Pya-Sar-CONH2,
MeCO-r-Abu-N-T-W—K(Ac)-aMeC-AEF-2Nal-THP-K(PEG2PEG2gEC12)-N-3Pya-Sar-CONH2,
MeCO-r-Abu-N-T-W—K(Ac)-aMeC-AEF-2Nal-THP-E-N-3Pya-Sar-K(PEG2PEG2gEC12)-CONH2,
MeCO-r-Abu-N-T-7MeW—K(PEG2PEG2gEC12)-aMeC-AEF-2Nal-THP-E-N-3Pya-Sar-CONH2,
MeCO-r-Abu-N-T-7MeW—K(Ac)-aMeC-AEF-2Nal-THP-E-N-3Pya-Sar-K(PEG2PEG2gEC12)-CONH2,
MeCO-r-Abu-N-T-W—K(PEG2PEG2gEC12)-aMeC-AEF-2Nal-THP-E-N-3Pya-Sar-CONH2,
MeCO-Pen-N-T-7MeW—K(Ac)-Pen-AEF(G)-2Nal-THP-K(PEG2PEG2gEC14)-N-3Pya-Sar-CONH2,
MeCO-Pen-N-T-7MeW—K(Ac)-Pen-AEF-2Nal-THP-E-N-3Pya-Sar-K(PEG2PEG2gEC12)-CONH2,
MeCO-Pen-N-T-7MeW—K(Ac)-Pen-AEF-2Nal-THP-E-N-3Pya-Sar-K(PEG2PEG2gEC14)-CONH2,
MeCO-Pen-K(PEG2PEG2gEC12)-T-7MeW—K(Ac)-Pen-AEF-2Nal-THP-E-N-3Pya-Sar-CONH2,
MeCO-r-Pen-N-T-7MeW—K(Ac)-Pen-AEF-2Nal-THP-E-N-3Pya-Sar-K(PEG2NMePEG2NMegENMeC18Tetrazole)-CONH2,
MeCO-Pen-K(C14)-T-7MeW—K(Ac)-Pen-AEF-2Nal-THP-E-N-3Pya-Sar-CONH2,
MeCO-Pen-N-T-7MeW—K(C14)-Pen-AEF-2Nal-THP-E-N-3Pya-Sar-CONH2,
MeCO-Pen-N-T-7MeW—K(Ac)-Pen-AEF-2Nal-THP-K(C14)-N-3Pya-Sar-CONH2,
MeCO-Pen-K(gEC14)-T-7MeW—K(Ac)-Pen-AEF-2Nal-THP-E-N-3Pya-Sar-CONH2,
MeCO-Pen-N-T-7MeW—K(gEC14)-Pen-AEF-2Nal-THP-E-N-3Pya-Sar-CONH2,
MeCO-Pen-N-T-7MeW—K(Ac)-Pen-AEF-2Nal-THP-K(gEC14)-N-3Pya-Sar-CONH2,
MeCO-Pen-N-T-7MeW—K(Ac)-Pen-AEF-2Nal-THP-K(Ac)—N-3Pya-Sar-K(PEG2PEG2gEC14)-CONH2,
MeCO-Pen-N-T-7MeW—K(Ac)-Pen-AEF-2Nal-THP-E-N-3Pya-Sar-K(gEC14)-CONH2,
MeCO-Pen-N-T-7MeW—K(Ac)-Pen-AEF-2Nal-THP-E-N-3Pya-Sar-K(gEC12)-CONH2,
MeCO-k(PEG2PEG2gEC12)-Pen-N-T-7MeW—K(Ac)-Pen-AEF-2Nal-THP-K(Ac)—N-3Pya-Sar-CONH2,
MeCO-k(PEG2PEG2gEC14)-Pen-N-T-7MeW—K(Ac)-Pen-AEF-2Nal-THP-K(Ac)—N-3Pya-Sar-CONH2,
MeCO-Pen-N-T-7MeW—K(Ac)-Pen-AEF-2Nal-THP-E-N-3Pya-Sar-K(C14)-CONH2,
MeCO-Pen-N-T-7MeW—K(Ac)-Pen-AEF-2Nal-THP-K(Ac)—N-3Pya-Sar-K(PEG2PEG2gEC12)-CONH2,
MeCO-Pen-N-T-7MeW—K(Ac)-Pen-AEF-2Nal-THP-E-N-3Pya-Sar-K(PEG2PEG2gEDProC14)-CONH2,
MeCO-Pen-N-T-7MeW—K(Ac)-Pen-AEF-2Nal-THP-E-N-3Pya-Sar-K(PEG2PEG2C14)-CONH2,
MeCO-Pen-N-T-7MeW—K(Ac)-Pen-AEF-2Nal-THP-E-N-3Pya-Sar-K(GSGSGSGC14)-CONH2,
MeCO-Pen-N-T-7MeW—K(Ac)-Pen-AEF-2Nal-THP-E-N-3Pya-Sar-K(PEG2PEG2SP6C14)-CONH2,
MeCO-Pen-N-T-7MeW—K(Ac)-Pen-AEF-2Nal-THP-E-N-3Pya-Sar-K(PEG2C14)-CONH2,
MeCO-Pen-N-T-7MeW—K(Ac)-Pen-AEF-2Nal-THP-E-N-3Pya-Sar-K(PEG2PEG2gESarC14)-CONH2, and
MeCO-Pen-N-T-7MeW—K(Ac)-Pen-AEF-2Nal-THP-E-N-3Pya-Sar-K(PEG2PEG2gEProC14)-CONH2,
PEG2PEG2-gEC16OH-r-Pen-N-T-7MeW—K(Ac)-Pen-AEF-2Nal-THP-K(Ac)—N-3Pya-Sar-CONH2,
PEG2_Palm-Pen-N-T-7MeW—K(Ac)-Pen-AEF-2Nal-THP-E-N-3Pya-Sar-CONH2,
PEG2_Lauryl-Pen-N-T-7MeW—K(Ac)-Pen-AEF-2Nal-THP-E-N-3Pya-Sar-CONH2,
PEG4_Palm-Pen-N-T-7MeW—K(Ac)-Pen-AEF-2Nal-THP-E-N-3Pya-Sar-CONH2,
PEG4_Lauryl-Pen-N-T-7MeW—K(Ac)-Pen-AEF-2Nal-THP-E-N-3Pya-Sar-CONH2,
PEG4_Capryl-Pen-N-T-7MeW—K(Ac)-Pen-AEF-2Nal-THP-E-N-3Pya-Sar-CONH2,
PEG4_Hexyl-Pen-N-T-7MeW—K(Ac)-Pen-AEF-2Nal-THP-E-N-3Pya-Sar-CONH2,
C12gEPEG2PEG2CO-Pen-N-T-7MeW—K(PEG2PEG2gEC12)-Pen-AEF-2Nal-THP-E-N-3Pya-Sar-CONH2,
C14gEPEG2PEG2CO-Pen-N-T-7MeW—K(PEG2PEG2gEC14)-Pen-AEF-2Nal-THP-E-N-3Pya-Sar-CONH2,
C14gEPEG2PEG2CO-k(PEG2PEG2gEC14)-Pen-N-T-7MeW—K(Ac)-Pen-AEF-2Nal-THP-E-N-3Pya-Sar-CONH2,
C12gEPEG2PEG2CO-k(PEG2PEG2gEC12)-Pen-N-T-7MeW—K(Ac)-Pen-AEF-2Nal-THP-E-N-3Pya-Sar-CONH2,
C14gEPEG2PEG2CO-Pen-N-T-7MeW—K(Ac)-Pen-AEF-2Nal-THP-E-N-3Pya-Sar-K(PEG2PEG2gEC14)-CONH2,
C12gEPEG2PEG2CO-Pen-N-T-7MeW—K(Ac)-Pen-AEF-2Nal-THP-E-N-3Pya-Sar-CONH2,
C14gEPEG2PEG2CO-Pen-N-T-7MeW—K(Ac)-Pen-AEF-2Nal-THP-E-N-3Pya-Sar-CONH2,
C12gEPEG2PEG2CO-Pen-K(PEG2PEG2gEC12)-T-7MeW—K(Ac)-Pen-AEF-2Nal-THP-E-N-3Pya-Sar-CONH2,
C14gEPEG2PEG2CO-Pen-K(PEG2PEG2gEC14)-T-7MeW—K(Ac)-Pen-AEF-2Nal-THP-E-N-3Pya-Sar-CONH2,
C12gEPEG2PEG2CO-Pen-N-T-7MeW—K(Ac)-Pen-AEF-2Nal-THP-K(PEG2PEG2gEC12)-N-3Pya-Sar-CONH2,
C14gEPEG2PEG2CO-Pen-N-T-7MeW—K(Ac)-Pen-AEF-2Nal-THP-K(PEG2PEG2gEC14)-N-3Pya-Sar-CONH2,
C12gEPEG2PEG2CO-r-Pen-N-T-7MeW—K(Ac)-Pen-AEF-2Nal-THP-E-N-3Pya-Sar-C12gEPEG2PEG2CO-r-Pen-N-T-7MeW—K(Ac)-Pen-AEF-2Nal-THP-E-N-3Pya-Sar-K(PEG2PEG2gEC12)-CONH2,
C12gEPEG2PEG2CO-r-Pen-N-T-7MeW—K(Ac)-Pen-AEF-2Nal-THP-K(PEG2PEG2gEC12)-N-3Pya-Sar-CONH2,
C12gEPEG2PEG2CO-Pen-N-T-7MeW—K(Ac)-Pen-AEF(G)-2Nal-THP-E-N-3Pya-Sar-CONH2,
C12gEPEG2PEG2CO-r-Pen-N-T-7MeW—K(PEG2PEG2gEC12)-Pen-AEF-2Nal-THP-E-N-3Pya-Sar-CONH2,
C12gEPEG2PEG2CO-Pen-N-T-7MeW—K(Ac)-Pen-AEF-2Nal-THP-K(Ac)—N-3Pya-Sar-CONH2, and
C14gEPEG2PEG2CO-Pen-N-T-7MeW—K(Ac)-Pen-AEF-2Nal-TIP-K(Ac)—N-3Pya-Sar-CONH2.

3. The polypeptide of claim 1 or claim 2, or a pharmaceutically acceptable salt thereof, having an amino acid sequence of Formula (I-A): 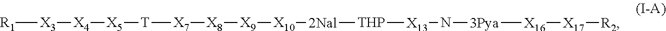
wherein:
R₁ is MeCO, Z_{lipid}, Z_{peg}, succiniccarn, 5cpaCO, or AEEP-Z_{lipid};
X₃ is r, K—Z_{lipid}, or absent;
X₄ is Pen or Abu;
X₅ is N, N(NMe2), K(d), or K—Z_{lipid};
X₇ is 7MeW, W, or absent;
X₈ is K(Ac), K(d), K(NmeAc), Q, K—Z_{lipid}, Dab(Nmecarn), Dab-Z_{peg};
X₉ is Pen or aMeC;
X₁₀ is AEF, TMAPF, AEF(d), APEG3F, TMAPF-Z_{lipid}, or AEF-Z_{lipid};
X₁₃ is E, K(Ac), K(d), K—Z_{peg}, K(NmeAc), Dab(Nmecarn), E(Oall), K—Z_{lipid} or absent;
X₁₆ is Sar, NmeK(d), or NmeK-Z_{lipid};
X₁₇ is K—Z_{lipid} or absent; and
R₂ is CONH2, CONMe₂, CONH—Z_{peg}, or CO—Z_{lipid};
wherein the peptide is cyclized via a disulfide or thioether linkage between the residues at X₄ and X₉; and
wherein the polypeptide comprises at least one lipid-containing amino acid.

4. The polypeptide of claim 1 or claim 2, or a pharmaceutically acceptable salt thereof, having an amino acid sequence of Formula (I-B): 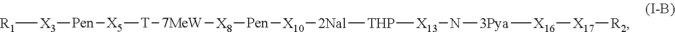
wherein:
R₁ is MeCO, Z_{lipid}, Z_{peg}, succiniccarn, or 5cpaCO;
X₅ is N, N(NMe2), or K—Z_{lipid};
X₈ is K(Ac), K(d), K(NmeAc), K—Z_{lipid}, Dab-Z_{peg};
X₁₀ is AEF, TMAPF, APEG3F, or AEF-Z_{lipid};
X₁₃ is E, K(Ac), K(d), K—Z_{peg}, K(NmeAc), or K—Z_{lipid};
X₁₆ is Sar, or NmeK-Z_{lipid};
X₁₇ is K—Z_{lipid} or absent; and
R₂ is CONH2, CONMe₂, or CONH—Z_{peg}.

5. The polypeptide of claim 1 or claim 2, or a pharmaceutically acceptable salt thereof, having an amino acid sequence of Formula (I-A): 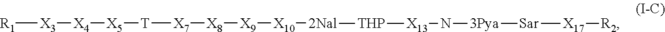
wherein:
R₁ is MeCO, Z_{lipid}, succiniccarn, 5cpaCO, or AEEP-Z_{lipid};
X₈ is K(Ac), K(d), K(NMeAc), K—Z_{lipid}, Dab(NMecarn); and
X₁₀ is AEF, TMAPF, TMAPF-Z_{lipid}, or AEF-Z_{lipid};
X₁₃ is E, K(Ac), K(d), K—Z_{peg}, K(NMeAc), or K—Z_{lipid}; and
X₁₇ is absent.

6. The polypeptide of claim 1 or claim 2, or a pharmaceutically acceptable salt thereof, having a sequence according to any one of Formulas I-D to I-G:

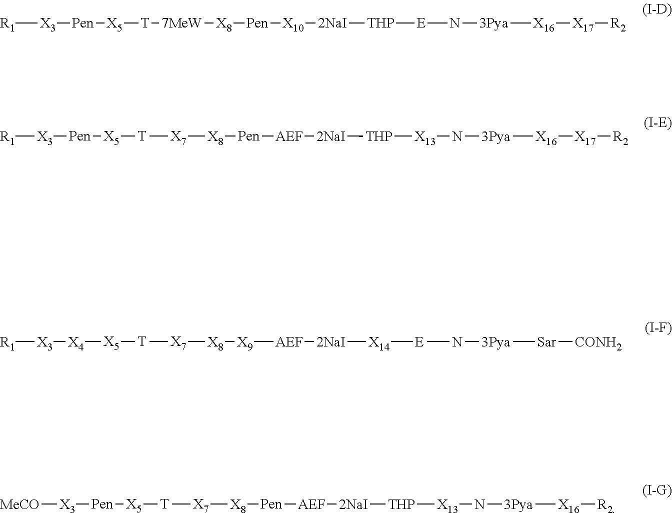

7. The polypeptide of any one of claims 1-6, or a pharmaceutically acceptable salt thereof, wherein each polyethylene glycol chain, independently, terminates in an ammonium group or a methyl group.

8. The polypeptide of any one of claims 1-6, or a pharmaceutically acceptable salt thereof, wherein each polyethylene glycol chain, independently, is: 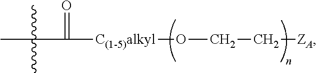
wherein:
Z_{A} is —OCH₃, —N⁺(CH₃)₃, 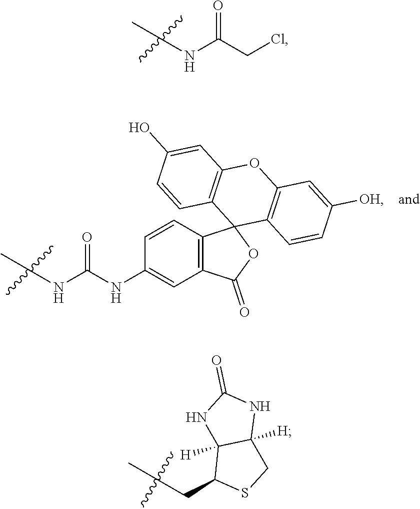
and
n is an integer from 2 to 15.

9. The polypeptide of any one of claims 1-8, or a pharmaceutically acceptable salt thereof, wherein each lipophilic substituent, independently for each occurrence, is: 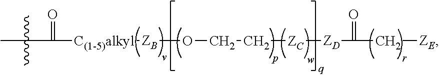
wherein:
Z_{B} is 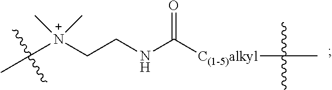
Z_{C} is Z_{C1}, Z_{C2}, or Z_{C3};
Z_{C1} is 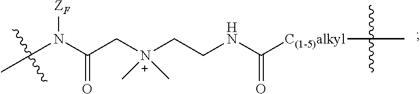
Z_{C2} is 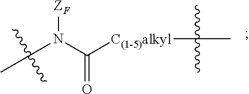
Z_{C3} is 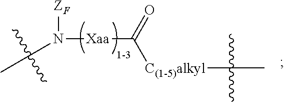
Z_{D} is 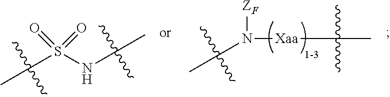
Z_{E} is —H, —COOH, phenoxy, or tetrazolyl;
Z_{F} is —H or —CH₃;
Xaa is, independently for each occurrence, 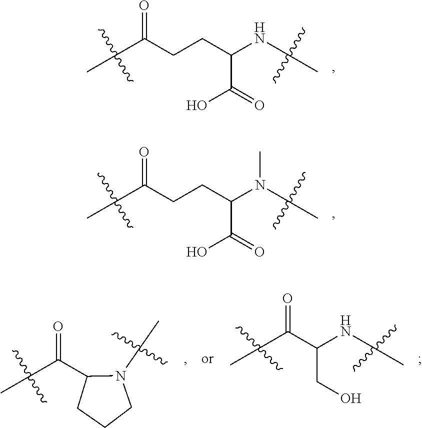
p, independently for each occurrence, is 0, 1, 2, 3, 4, 5, or 6;
q is 1, 2, 3, 4, 5, or 6;
r is an integer from 6 to 24;
v is 0 or 1; and
w, independently for each occurrence, is 0 or 1.

10. The polypeptide of any one of claims 1-9, or a pharmaceutically acceptable salt thereof, wherein:
Z_{lipid}, independently for each occurrence, is: 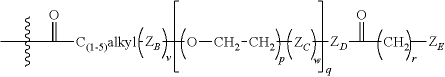
Z_{C2} is 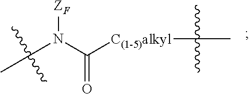
Z_{D} is 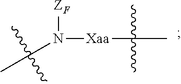
Z_{E} is —H, —COOH, or tetrazolyl;
Z_{F} is —H or —CH₃;
Xaa is 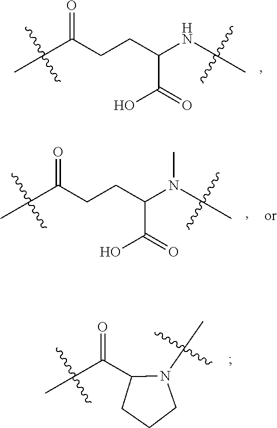 [SMILES: *NC(CCC(*)=O)C(=O)O ; *C(=O)CCC(C(=O)O)N(*)C ; *C(=O)C1CCCN1*]
n is an integer from 2 to 15;
p, independently for each occurrence, is 1, 2, 3, 4, 5, or 6;
q is 1, 2, 3, 4, 5, or 6;
r is an integer from 6 to 24; and
w, independently for each occurrence, is 0 or 1.

11. The polypeptide of any one of claims 1-10, or a pharmaceutically acceptable salt thereof, wherein R₁, R₂, or any amino acid in the amino acid sequence is conjugated to a lipophilic substituent.

12. The polypeptide of any one of claims 1-11, or a pharmaceutically acceptable salt thereof, wherein:
X₄ is Pen and X₉ is Pen; or
X₄ is Abu and X₉ is C or aMeC.

13. The polypeptide of claim 12, or a pharmaceutically acceptable salt thereof, wherein the peptide comprises a disulfide or thioether linkage between X₄ and X₉.

14. The polypeptide of any one of claims 1-13, or a pharmaceutically acceptable salt thereof, wherein X₄ is Pen(oXyl), Pen(mXyl), or Pen(pXyl) and X₉ is Pen.

15. The polypeptide of claim 14, or a pharmaceutically acceptable salt thereof, wherein the linkage between X₄ and X₉ has the structure:

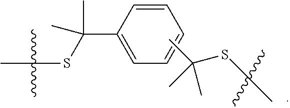

16. The polypeptide of any one of claims 1-11, or a pharmaceutically acceptable salt thereof, wherein X₄ is 4AminoPro and X₉ is D, E, or hE.

17. The polypeptide of claim 16, or a pharmaceutically acceptable salt thereof, wherein the linkage between X₄ and X₉ has the structure:

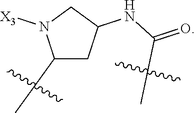

18. The polypeptide of any one of claims 1-11, or a pharmaceutically acceptable salt thereof, wherein X₄ is Dap and X₉ is D.

19. The polypeptide of claim 18, or a pharmaceutically acceptable salt thereof, wherein linkage between X₄ and X₉ is an amide linkage.

20. The polypeptide of any one of claims 1-11, or a pharmaceutically acceptable salt thereof, wherein X₄ is Pra and X₉ is Dap.

21. The polypeptide of claim 20, or a pharmaceutically acceptable salt thereof, wherein the peptide is cyclized via a triazole linkage.

22. The polypeptide of any one of claims 1-11, or a pharmaceutically acceptable salt thereof, wherein X₄ is aG and X₉ is aG.

23. The polypeptide of claim 22, or a pharmaceutically acceptable salt thereof, wherein the peptide is cyclized via an aliphatic linkage.

24. The polypeptide of any one of claims 1-23, or a pharmaceutically acceptable salt thereof, wherein the polypeptide comprises two lipophilic substituents.

25. The polypeptide of any of claims 1-23, or a pharmaceutically acceptable salt thereof, wherein the polypeptide comprises no more than one lipophilic substituent.

26. The polypeptide of any of claims 1-25, or a pharmaceutically acceptable salt thereof, wherein R₁ is MeCO.

27. The polypeptide of any of claims 1-26, or a pharmaceutically acceptable salt thereof, wherein R₂ is CONH2.

28. The polypeptide of any one of claims 1, 7-11, or 25-27, wherein the ring comprises 4-9 or 11 amino acids.

29. The polypeptide of any one of claims 1, 7-11, or 25-27, wherein the ring is formed between X₄ and X₉, X₄ and X₁₃, X₅ and X₁₀, X₃ and X₁₃, or X₆ and X₉.

30. The polypeptide of claim 29, wherein the ring is formed between X₄ and X₉, X₄ and X₁₃, or X₆ and X₉.

31. The polypeptide of claim 30, wherein the ring is formed between X₄ and X₉ via a linker having one or more groups selected from the group consisting of a disulfide, thioether, amide, olefin, ether, alkylene, and triazole.

32. The polypeptide of claim 30, wherein the ring is formed between X₄ and X₁₃ via a linker having one or more groups selected from the group consisting of a disulfide, thioether, amide, olefin, ether, alkylene, and triazole.

33. The polypeptide of claim 30, wherein the ring is formed between X₆ and X₉ via a linker having one or more groups selected from the group consisting of a disulfide, thioether, amide, olefin, ether, alkylene, and triazole.

34. A polypeptide having an amino acid sequence selected from the group consisting of SEQ ID NOS: 1-162.

35. A pharmaceutical composition comprising a polypeptide of any one of claims 1-34, or a pharmaceutically acceptable salt thereof, and a pharmaceutically acceptable carrier.

36. A method for treating a disease or disorder associated with Interleukin 23 (IL-23)/Interleukin 23 Receptor (TL-23R), comprising administering to a subject in need thereof a therapeutically effective amount of a polypeptide of any one of claims 1-34 or a pharmaceutical composition of claim 35.

37. The method of claim 36, wherein the disease or disorder is selected from multiple sclerosis, asthma, rheumatoid arthritis, inflammation of the gut, inflammatory bowel diseases (IBDs), juvenile IBD, adolescent IBD, Crohn's disease, ulcerative colitis, Celiac disease (nontropical Sprue), microscopic colitis, collagenous colitis, eosinophilic gastroenteritis/esophagitis, colitis associated with radio- or chemo-therapy, colitis associated with disorders of innate immunity as in leukocyte adhesion deficiency-1, sarcoidosis, Systemic Lupus Erythematosus, ankylosing spondylitis (axial spondyloarthritis), psoriatic arthritis, psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, enteropathy associated with seronegative arthropathies, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.

38. The method of claim 36, wherein the disease or disorder is selected from ulcerative colitis (UC), Crohn's disease (CD), psoriasis (PsO), or psoriatic arthritis (PsA).
